(19) United States
(12) Patent Application Publication
(10) Pub. No.: US 2019/0335726 A1
(43) Pub. Date: Nov. 7, 2019

(54) GENETICALLY MODIFIED CELLS, TISSUES, AND ORGANS FOR TREATING DISEASE

(71) Applicant: Regents of the University of Minnesota, Minneapolis, MN (US)

(72) Inventors: Bernhard J. Hering, Minnetonka, MN (US); Christopher Burlak, Minnetonka, MN (US)

(21) Appl. No.: 16/312,052

(22) PCT Filed: Jun. 14, 2017
(86) PCT No.: PCT/US2017/037566

§ 371 Date: Dec. 20, 2018

Related U.S. Application Data
(60) Provisional application No. 62/350,048, filed on Jun. 14, 2016.

(51) Int. Cl. A01K 67/027; C12N 15/85

(52) U.S. Cl. CPC A01K 67/0278; C12N 15/8509; A01K 2217/075; C12N 2015/8518; A01K 2227/106; A01K 2227/108; A01K 2267/025; A01K 2217/15

(57) ABSTRACT

Genetically modified cells, tissues, and organs for treating or preventing diseases are disclosed. Also disclosed are methods of making the genetically modified cells and non-human animals.

### CROSS-REFERENCE

This application claims the benefit of U.S. Provisional Application No. 62/350,048, filed Jun. 14, 2016, which application is incorporated herein by reference in its entirety.

### BACKGROUND OF THE DISCLOSURE

There is a shortage of organs, tissues or cells available for transplantation in recipients such as humans. Xenotransplantation or allotransplantation of organs, tissues, or cells into humans has the potential to fulfill this need and help hundreds of thousands of people every year. Non-human animals can be chosen as organ donors based on their anatomical and physiological similarities to humans. Additionally, xenotransplantation has implications not only in humans, but also in veterinary applications.

However, unmodified wild-type non-human animal tissues can be rejected by recipients, such as humans, by the immune system. Rejection is believed to be caused at least in part by antibodies binding to the tissues and cell-mediated immunity leading to graft loss. For example, pig grafts can be rejected by cellular mechanisms mediated by adaptive immune cells.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY

In a first aspect, disclosed herein are genetically modified non-human animals comprising an exogenous nucleic acid sequence at least 95% identical to SEQ ID NO: 359 or SEQ ID NO: 502.

In some embodiments of the first aspect, the exogenous nucleic acid is at least 96% identical to SEQ ID NO: 359 or SEQ ID NO: 502. In some embodiments, the exogenous nucleic acid is at least 97% identical to SEQ ID NO: 359 or SEQ ID NO: 502. In some embodiments, the exogenous nucleic acid is at least 98% identical to SEQ ID NO: 359 or SEQ ID NO: 502. In some embodiments, the exogenous nucleic acid is at least 99% identical to SEQ ID NO: 359 or SEQ ID NO: 502. In some embodiments, the exogenous nucleic acid is 100% identical to SEQ ID NO: 359 or SEQ ID NO: 502.

In a second aspect, disclosed herein are genetically modified non-human animals comprising an exogenous nucleic acid that is transcribed as a human leukocyte antigen G (HLA-G) mRNA with a modified 3′ untranslated region.

In some embodiments of the second aspect, the modified 3′ untranslated region comprises one or more deletions. In some embodiments, the modified 3′ untranslated region increases stability of the mRNA in comparison to an unmodified HLA-G mRNA. In some embodiments, the HLA-G is HLA-G1, HLA-G2, HLA-G3, HLA-G4, HLA-G5, HLA-G6, or HLA-G7. In some embodiments, the HLA-G is HLA-G1. In some embodiments, the HLA-G is HLA-G2.

In some embodiments of the first or second aspect, at least one cell of the genetically modified non-human animal expresses a HLA-G protein. In some embodiments, the HLA-G protein is HLA-G1.

Some embodiments of the first or second aspect further comprise a second exogenous nucleic acid that encodes for a β-2-microglobulin (B2M) protein. In some embodiments, the B2M protein is a human B2M protein.

In a third aspect, disclosed herein are genetically modified non-human animals comprising an exogenous nucleic acid sequence at least 75% identical to SEQ ID NO: 240.

In some embodiments of the third aspect, the exogenous nucleic acid sequence is at least 80% identical to SEQ ID NO: 240. In some embodiments, the exogenous nucleic acid sequence is at least 85% identical to SEQ ID NO: 240. In some embodiments, the exogenous nucleic acid sequence is at least 90% identical to SEQ ID NO: 240. In some embodiments, the exogenous nucleic acid sequence is at least 95% identical to SEQ ID NO: 240. In some embodiments, the exogenous nucleic acid sequence is identical to SEQ ID NO: 240.

In some embodiments of the third aspect, at least one cell of the genetically modified non-human animal expresses a human CD47 protein.

Some embodiments of the third aspect further comprise a second exogenous nucleic acid sequence that is transcribed as a human leukocyte antigen G (HLA-G) mRNA with a modified 3′ untranslated region. In some embodiments, the modified 3′ untranslated region comprises one or more deletions. In some embodiments, the modified 3′ untranslated region increases stability of the mRNA in comparison to an unmodified HLA-G mRNA. In some embodiments, the HLA-G is HLA-G1, HLA-G2, HLA-G3, HLA-G4, HLA-G5, HLA-G6, or HLA-G7. In some embodiments, the HLA-G is HLA-G1. In some embodiments, the HLA-G is HLA-G2.

In some embodiments of the third aspect, the second exogenous nucleic acid sequence is at least 95%, 96%, 97%, 98%, 99%, or 100% identical to SEQ ID NO: 359 or SEQ ID NO: 502.

In some embodiments of the first, second, or third aspects, the exogenous nucleic acid sequence is operatively linked to a constitutively active endogenous promoter.

In some embodiments of the first, second, or third aspects, the exogenous nucleic acid sequence is inserted in the genetically modified non-human animal's genome at a ROSA 26 gene site.

In some embodiments of the first, second, or third aspects, the exogenous nucleic acid sequence is inserted in the genetically modified non-human animal's genome at a site effective to reduce expression of a glycoprotein galactosyltransferase alpha 1,3 (GGTA1), a putative cytidine monophosphate-N-acetylneuraminic acid hydroxylase-like protein (CMAH), a β1,4 N-acetylgalactosaminyltransferase (B4GALNT2), a C—X—C motif chemokine 10 (CXCL10), a MHC class I polypeptide-related sequence A (MICA), a MHC class I polypeptide-related sequence B (MICB), a transporter associated with antigen processing 1 (TAP1), a NOD-like receptor family CARD domain containing 5 (NLRC5), or a combination thereof in comparison to: an animal of the same species without the exogenous nucleic acid sequence or an animal of the same species with the exogenous nucleic acid inserted in a different site.

In some embodiments of the first, second, or third aspects, the exogenous nucleic acid sequence is inserted in the genetically modified non-human animal's genome at the site effective to reduce expression of the glycoprotein galactosyltransferase alpha 1,3 (GGTA1).

In some embodiments of the first, second, or third aspects, the genetically modified non-human animal further comprises a genomic disruption in one or more genes selected from the list consisting of: a glycoprotein galactosyltransferase alpha 1,3 (GGTA1), a putative cytidine monophosphate-N-acetylneuraminic acid hydroxylase-like protein (CMAH), a β1,4 N-acetylgalactosaminyltransferase (B4GALNT2), a C—X—C motif chemokine 10 (CXCL10), a MHC class I polypeptide-related sequence A (MICA), a MHC class I polypeptide-related sequence B (MICB), a transporter associated with antigen processing 1 (TAP1), a NOD-like receptor family CARD domain containing 5 (NLRC5), and any combination thereof.

In some embodiments of the first, second, or third aspects, the genetically modified non-human animal further comprises a genomic disruption in one or more genes selected from the list consisting of: a component of an MHC I-specific enhanceosome, a transporter of an MHC I-binding peptide, a natural killer (NK) group 2D ligand, a CXC chemokine receptor (CXCR)3 ligand, MHC II transactivator (CIITA), C3, an endogenous gene not expressed in a human, and any combination thereof. Some embodiments comprise the genomic disruption of the component of a MHC I-specific enhanceosome, wherein the component of a MHC I-specific enhanceosome is NOD-like receptor family CARD domain containing 5 (NLRC5). Some embodiments comprise the genomic disruption of the transporter of a MHC I-binding peptide, wherein the transporter is transporter associated with antigen processing 1 (TAP1). Some embodiments comprise the genomic disruption of C3. Some embodiments comprise the genomic disruption of the NK group 2D ligand, wherein the NK group 2D ligand is MHC class I polypeptide-related sequence A (MICA) or MHC class I polypeptide-related sequence B (MICB). Some embodiments comprise the genomic disruption of the endogenous gene not expressed in a human, wherein the endogenous gene not expressed in a human is glycoprotein galactosyltransferase alpha 1,3 (GGTA1), putative cytidine monophosphate-N-acetylneuraminic acid hydroxylase-like protein (CMAH), or β1,4 N-acetylgalactosaminyltransferase (B4GALNT2). Some embodiments comprise the genomic disruption of the CXCR3 ligand, wherein the CXCR3 ligand is C—X—C motif chemokine 10 (CXCL10).

In some embodiments, the genomic disruption reduces expression of the disrupted gene in comparison to an animal of the same species without the genomic disruption.

In some embodiments, the the genomic disruption reduces protein expression from the disrupted gene in comparison to an animal of the same species without the genomic disruption.

Some embodiments of the first, second, or third aspect further comprise an additional exogenous nucleic acid sequence encoding an infected cell protein 47 (ICP47).

In some embodiments of the first, second, or third aspect, the genetically modified non-human animal is a member of the Laurasiatheria superorder.

In some embodiments of the first, second, or third aspect, the genetically modified non-human animal is an ungulate.

In some embodiments of the first, second, or third aspect, the genetically modified non-human animal is a pig.

In some embodiments of the first, second, or third aspect, the genetically modified non-human animal is a non-human primate.

In some embodiments of the first, second, or third aspect, the genetically modified non-human animal is fetus.

Also disclosed herein are cells isolated from the genetically modified non-human animal of any embodiments of the first, second, or third aspects. In some embodiments, the cell is an islet cell. In some embodiments, the cell is a stem cell.

Also disclosed herein are tissues isolated from the genetically modified non-human animal of any embodiments of the first, second, or third aspects. In some embodiments, the tissue is a solid organ transplant. In some embodiments, the tissue is all or a portion of a liver. In some embodiments, the tissue is all or a portion of a kidney.

In a fourth aspect, disclosed herein are non-human cells comprising an exogenous nucleic acid sequence at least 95% identical to SEQ ID NO: 359 or SEQ ID NO: 502.

In some embodiments of the fourth aspect, the exogenous nucleic acid is at least 96% identical to SEQ ID NO: 359 or SEQ ID NO: 502. In some embodiments, the exogenous nucleic acid is at least 97% identical to SEQ ID NO: 359 or SEQ ID NO: 502. In some embodiments, the exogenous nucleic acid is at least 98% identical to SEQ ID NO: 359 or SEQ ID NO: 502. In some embodiments, the exogenous nucleic acid is at least 90% identical to SEQ ID NO: 359 or SEQ ID NO: 502. In some embodiments, the exogenous nucleic acid is 100% identical to SEQ ID NO: 359 or SEQ ID NO: 502.

In some embodiments of the fourth aspect, the non-human cell expresses human leukocyte antigen G1 (HLA-G1) on the cell surface.

In a fifth aspect, disclosed herein are non-human cells comprising an exogenous nucleic acid that is transcribed as a human leukocyte antigen G (HLA-G) mRNA with a modified 3′ untranslated region.

In some embodiments of the fifth aspect, the modified 3′ untranslated region comprises one or more deletions. In some embodiments, the modified 3′ untranslated region increases stability of the mRNA in comparison to an unmodified HLA-G mRNA.

In some embodiments of the fifth aspect, the HLA-G is HLA-G1, HLA-G2, HLA-G3, HLA-G4, HLA-G5, HLA-G6, or HLA-G7. In some embodiments, the HLA-G is HLA-G1. In some embodiments, the HLA-G is HLA-G2.

In some embodiments of the fourth or fifth aspect, the non-human cell further comprises a second exogenous nucleic acid that encodes for a β-2-microglobulin (B2M) protein. In some embodiments, the B2M protein is a human B2M protein.

In a sixth aspect, disclosed herein are non-human cells comprising an exogenous nucleic acid at least 75% identical to SEQ ID NO: 240.

In some embodiments of the sixth aspect, the exogenous nucleic acid sequence is at least 80% identical to SEQ ID NO: 240.

In some embodiments, the exogenous nucleic acid sequence is at least 85% identical to SEQ ID NO: 240. In some embodiments, the exogenous nucleic acid sequence is at least 90% identical to SEQ ID NO: 240. In some embodiments, the exogenous nucleic acid sequence is at least 95% identical to SEQ ID NO: 240. In some embodiments, the exogenous nucleic acid sequence is 100% identical to SEQ ID NO: 240.

In some embodiments of the sixth aspect, the at least one non-human cell expresses a human CD47 protein.

In some embodiments of the sixth aspect, the non-human cell further comprises a second exogenous nucleic acid sequence that is transcribed as a human leukocyte antigen G (HLA-G) mRNA with a modified 3′ untranslated region. In some embodiments, the modified 3′ untranslated region comprises one or more deletions. In some embodiments, the modified 3′ untranslated region increases stability of the mRNA in comparison to an unmodified HLA-G mRNA. In some embodiments, the HLA-G is HLA-G1, HLA-G2, HLA-G3, HLA-G4, HLA-G5, HLA-G6, or HLA-G7. In some embodiments, the HLA-G is HLA-G1. In some embodiments, the HLA-G is HLA-G2. In some embodiments, the second exogenous nucleic acid sequence is at least 95%, 96%, 97%, 98%, 99%, or 100% identical to SEQ ID NO: 359 or SEQ ID NO: 502.

In some embodiments of the fourth, fifth, or sixth aspects, the exogenous nucleic acid sequence is operatively linked to a constitutively active endogenous promoter.

In some embodiments of the fourth, fifth, or sixth aspects, the exogenous nucleic acid sequence is inserted in the non-human cell's genome at a ROSA 26 gene site.

In some embodiments of the fourth, fifth, or sixth aspects, the exogenous nucleic acid sequence is inserted in the non-human cell's genome at a site effective to reduce expression of a glycoprotein galactosyltransferase alpha 1,3 (GGTA1), a putative cytidine monophosphate-N-acetylneuraminic acid hydroxylase-like protein (CMAH), a β1,4 N-acetylgalactosaminyltransferase (B4GALNT2), a C—X—C motif chemokine 10 (CXCL10), a MHC class I polypeptide-related sequence A (MICA), a MHC class I polypeptide-related sequence B (MICB), a transporter associated with antigen processing 1 (TAP1), a NOD-like receptor family CARD domain containing 5 (NLRC5), or a combination thereof in comparison to: a cell of the same species without the exogenous nucleic acid sequence or a cell of the same species wherein the exogenous nucleic acid is inserted in a different site.

In some embodiments of the fourth, fifth, or sixth aspects, the exogenous nucleic acid sequence is inserted in the non-human cell's genome at a site that reduces expression of a glycoprotein galactosyltransferase alpha 1,3 (GGTA1).

In some embodiments of the fourth, fifth, or sixth aspects, the non-human cell further comprises a genomic disruption in one or more genes selected from the list consisting of: a glycoprotein galactosyltransferase alpha 1,3 (GGTA1), a putative cytidine monophosphate-N-acetylneuraminic acid hydroxylase-like protein (CMAH), a β1,4 N-acetylgalactosaminyltransferase (B4GALNT2), a C—X—C motif chemokine 10 (CXCL10), a MHC class I polypeptide-related sequence A (MICA), a MHC class I polypeptide-related sequence B (MICB), a transporter associated with antigen processing 1 (TAP1), a NOD-like receptor family CARD domain containing 5 (NLRC5), and any combination thereof.

In some embodiments of the fourth, fifth, or sixth aspects, the non-human cell further comprises a genomic disruption in one or more genes selected from the list consisting of: a component of an MHC I-specific enhanceosome, a transporter of an MHC I-binding peptide, a natural killer (NK) group 2D ligand, a CXC chemokine receptor (CXCR)3 ligand, MHC II transactivator (CIITA), C3, an endogenous gene not expressed in a human, and any combination thereof. In some embodiments, the non-human cell comprises the genomic disruption of the component of a MHC I-specific enhanceosome, wherein the component of a MHC I-specific enhanceosome is NOD-like receptor family CARD domain containing 5 (NLRC5). In some embodiments, the non-human cell comprises the genomic disruption of the transporter of a MHC I-binding peptide, wherein the transporter is transporter associated with antigen processing 1 (TAP1)

In some embodiments, the non-human cell comprises the genomic disruption of C3.

In some embodiments, the non-human cell comprises the genomic disruption of the NK group 2D ligand, wherein the NK group 2D ligand is MHC class I polypeptide-related sequence A (MICA) or MHC class I polypeptide-related sequence B (MICB).

In some embodiments, the non-human cell comprises the genomic disruption of the endogenous gene not expressed in a human, wherein the endogenous gene not expressed in a human is glycoprotein galactosyltransferase alpha 1,3 (GGTA1), putative cytidine monophosphate-N-acetylneuraminic acid hydroxylase-like protein (CMAH), or β1,4 N-acetylgalactosaminyltransferase (B4GALNT2). In some embodiments, the non-human cell comprises the genomic disruption of a CXCR3 ligand, wherein the CXCR3 ligand is C—X—C motif chemokine 10 (CXCL10). In some embodiments, the genomic disruption reduces expression of the disrupted gene in comparison to a cell from the same species without the genomic disruption.

In some embodiments, the genomic disruption reduces protein expression from the disrupted gene in comparison to a cell from the same species without the genomic disruption.

Some embodiments of the fourth, fifth, or sixth aspects further comprise an additional exogenous nucleic acid sequence encoding an infected cell protein 47 (ICP47).

In some embodiments of the fourth, fifth, or sixth aspects, the non-human cell is a Laurasiatheria superorder cell.

In some embodiments of the fourth, fifth, or sixth aspects, the non-human cell is an ungulate cell.

In some embodiments of the fourth, fifth, or sixth aspects, the non-human cell is a pig cell.

In some embodiments of the fourth, fifth, or sixth aspects, the non-human cell is a non-human primate cell.

In some embodiments of the fourth, fifth, or sixth aspects, the non-human cell is a fetal cell.

In some embodiments of the fourth, fifth, or sixth aspects, the non-human cell is a stem cell.

In some embodiments of the fourth, fifth, or sixth aspects, the non-human cell is an islet cell.

Also disclosed herein are solid organ transplants comprising the non-human cell of any embodiment of the fourth, fifth, or sixth aspects.

Also disclosed herein are embryos comprising the non-human cell of any embodiment of the fourth, fifth, or sixth aspects.

In a seventh aspect, disclosed herein are methods comprising providing to a subject, at least one non-human cell of any embodiment of the fourth, fifth, or sixth aspects. In some embodiments, the at least one non-human cell is a solid organ transplant. In some embodiments, the at least one non-human cell is a stem cell transplant. In some embodiments, the at least one non-human cell is an islet cell transplant.

Some embodiments of the seventh aspect comprise providing to the subject a tolerizing vaccine. In some embodiments, the tolerizing vaccine is provided prior to, concurrently with, or after the at least one non-human cell is provided to the subject. In some embodiments, the tolerizing vaccine comprises apoptotic cells. In some embodiments, the tolerizing vaccine comprises cells from the same species as the at least one non-human cell provided to the subject. In some embodiments, the tolerizing vaccine comprises cells that are genetically identical to the at least one non-human cell provided to the subject.

Some embodiments of the seventh aspect comprise providing an anti-CD40 antibody to the subject. In some embodiments, the anti-CD40 antibody is provided prior to, concurrently with, or after the at least one non-human cell is provided to the subject. In some embodiments, the anti-CD40 antibody specifically binds to an epitope within amino acid sequence SEQ ID NO: 487. In some embodiments, the anti-CD40 antibody specifically binds to an epitope within amino acid sequence SEQ ID NO: 488.

In an eight aspect, disclosed herein are systems for xenotransplantation comprising: a) at least one cell isolated from the genetically modified non-human animal of any embodiment of the first, second or third aspects; and b) a tolerizing vaccine, anti-CD40 antibody, or a combination thereof. In some embodiments, the at least one cell comprises an islet cell, a stem cell, or a combination thereof. In some embodiments, the at least one cell is a solid organ transplant. In some embodiments, the at least one cell is all or a portion of a liver. In some embodiments, the at least one cell is all or a portion of a kidney.

Some embodiments of the eighth aspect comprise the tolerizing vaccine. In some embodiments, the tolerizing vaccine comprises apoptotic cells. In some embodiments, the tolerizing vaccine comprises cells from the same species as the at least one cell. In some embodiments, the tolerizing vaccine comprises cells that are genetically identical to the at least one cell.

Some embodiments of the eighth aspect comprise or further comprise the anti-CD40 antibody. In some embodiments, the anti-CD40 antibody specifically binds to an epitope within amino acid sequence SEQ ID NO: 487. In some embodiments, the anti-CD40 antibody specifically binds to an epitope within amino acid sequence SEQ ID NO: 488.

In a ninth aspect, disclosed herein are systems for xenotransplantation comprising: a) at least one non-human cell of any one of claims 58-108; and b) a tolerizing vaccine, an anti-CD40 antibody, or a combination thereof. In some embodiments, the at least one cell comprises an islet cell, a stem cell, or a combination thereof. In some embodiments, the at least one cell is a solid organ transplant. In some embodiments, the at least one cell is all or a portion of a liver. In some embodiments, the at least one cell is all or a portion of a kidney.

Some embodiments of the ninth aspect comprise the tolerizing vaccine. In some embodiments, the tolerizing vaccine comprises apoptotic cells. In some embodiments, the tolerizing vaccine comprises cells from the same species as the at least one cell. In some embodiments, the tolerizing vaccine comprises cells that are genetically identical to the at least one cell.

Some embodiments of the ninth aspect comprise or further comprise the anti-CD40 antibody. In some embodiments, the anti-CD40 antibody specifically binds to an epitope within amino acid sequence SEQ ID NO: 487. In some embodiments, the anti-CD40 antibody specifically binds to an epitope within amino acid sequence SEQ ID NO: 488.

Provided herein are methods comprising providing to an individual at least one engineered cell; wherein said engineered cell comprises at least two genomic modification resulting in inhibition of the immune response of said individual to said at least one engineered cell as measured by reduced effector function of at least one endogenous cell selected from a group consisting of T cells, B cells, monocytes, macrophages, Natural Killer (NK) cells, dendritic cells, and a combination thereof; and/or by increased immune cell regulation of at least one endogenous cell selected from a group including but not limited to CD4+ regulatory T cells, CD8+ regulatory T cells, CD8+ natural suppressor cells, Tr1 cells, regulatory B cells, B10 cells, myeloid-derived suppressor cells, and any combination thereof, as compared to an immune response of an individual contacted with a non-engineered counterpart cell. In some cases, the at least one engineered cell can be a solid organ transplant. In other cases, at least one engineered cell can be a stem cell transplant. In some cases, at least one engineered cell can be an islet cell transplant. An individual can be tolerized to an at least one engineered cell. In some cases, tolerization can occur before, during or, after an at least one engineered cell can be provided to an individual.

In some cases, tolerization can be facilitated by an administration of a vaccine. In some cases, tolerization can be an administration of at least one engineered cell. In some cases, tolerization can be an administration of a vaccine and administration of at least one engineered cell. A vaccine can comprise apoptotic cells. A vaccine can also comprise viable cells. In some cases, reduced effector function can be selected from a group consisting of reduced proliferation; reduced cytokine expression, reduced expression of cytolytic effector molecules, reduced persistence, deletion, induction of anergy, increased immune cell regulation, and any combination thereof in response to exposure to said at least one engineered cell.

Disclosed herein can also be administering to an individual at least one additional treatment step. In some cases, at least one additional treatment step can be an immunosuppressive therapy. An immunosuppressive therapy can be selected from a group consisting of an anti-CD40 antibody, an anti-CD20 antibody, an anti-IL6 receptor antibody, C₅₁H₇₉NO₁₃ (Rapamycin), soluble tumor necrosis factor receptor (sTNFR), C₆₆H₉₉N₂₃O₁₇S₂ (compstatin), and any combination thereof. An individual may not be sensitized to a major histocompatibility complex (MHC). The anti-CD40 antibody can be an antagonistic antibody. The anti-CD40 antibody can be an anti-CD40 antibody that specifically binds to an epitope within the amino acid sequence: EPPTACREKQYLINSQCCSLCQPGQKLVSDCTEFTETECLPCGESEFLDTWNRETHCHQH KYCDPNLGLRVQQKGTSETDTICTCEEGWHCTSEACESCV (SEQ ID NO: 487). The anti-CD40 antibody can be an anti-CD40 antibody that specifically binds to an epitope within the amino acid sequence: EKQYLINSQCCSLCQPGQKLVSDCTEFTETECL (SEQ ID NO: 488). The anti-CD40 antibody can be a Fab′ anti-CD40L monoclonal antibody fragment CDP7657. The anti-CD-40 antibody can be a FcR-engineered, Fc silent anti-CD40L monoclonal domain antibody. In some cases, an individual can be sensitized to a major histocompatibility complex (MHC). MHC can be human leukocyte antigen (HLA). An individual can be sensitized to a major histocompatibility complex (MHC) as determined by a positive response to a panel reactive antibody (PRA) screen analysis.

In some cases, an individual can have a calculated panel reactive antibody (cPRA) score from 0.1 to 100%. In some cases, a reduced effector function can be a reduced effector function of at least two endogenous cell types selected from a group consisting of T cells, B cells, monocytes, macrophages, Natural Killer (NK) cells, dendritic cells, and any combination thereof. A genome modification can be a gene disruption, deletion, induction of anergy, increased immune cell regulation, or a combination thereof. A gene can be selected from a group consisting of a C—X—C motif chemokine 10 (CXCL10), transporter associated with antigen processing 1 (TAP1), NOD-like receptor family CARD domain containing 5 (NLRC5), and any combination thereof. In some cases, an at least one engineered cell is a xenograft.

Disclosed herein can be an engineered polynucleic acid comprising at least two sequences encoding targeting oligonucleotides; wherein said targeting oligonucleotides comprise complementary sequences to at least one non-human genome sequence adjacent to a protospacer adjacent motif (PAM) sequence. In some cases, targeting oligonucleotides can be guide RNAs (gRNAs). A gRNA can comprise complementary sequences to a gene selected from a group consisting of GGTA1, Gal2-2, NLRC5, and any combination thereof. In some cases, gRNAs can comprise complementary sequences to GGTA1 and/or Gal2. A gRNA can comprise complementary sequences to NLRC5 and Gal2. In some cases, a targeting oligonucleotide can bind a first exon of said gene. A non-human genome can be a Laurasiatheria superorder animal or can be from a non-human primate. A Laurasiatheria super order animal can be an ungulate. In some cases, an ungulate can be a pig. A PAM sequence can be 5′-NGG-3′ (SEQ ID NO: 265).

In some cases, a guide RNA can comprise at least one modification. A modification can be selected from a group consisting of 5′adenylate, 5′ guanosine-triphosphate cap, 5′N⁷-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencer 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-0-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, and any combination thereof.

Disclosed herein can be a graft for xenotransplantation comprising at least one genomic disruption of SEQ ID NO: 261.

Disclosed herein can be a graft for xenotransplantation comprising at least one genomic disruption of SEQ ID NO: 262.

In some cases, a graft for xenotransplantation can further comprise at least one transgene. A transgene can be endogenous. A transgene can be engineered. A transgene can encode a human leukocyte antigen (HLA). An HLA can be HLA-G. A transgene can be CD47.

Provided herein is a genetically modified animal having a genomic disruption in two or more genes selected from a group consisting of: a component of an MHC I-specific enhanceosome, a transporter of an MHC I-binding peptide, a natural killer (NK) group 2D ligand, a CXC chemokine receptor (CXCR)3 ligand, MHC II transactivator (CIITA), C3, an endogenous gene not expressed in a human, and any combination thereof, wherein said genetically modified animal has reduced expression of said gene in comparison to a non-genetically modified counterpart animal. In some cases, a genetically modified animal can be a member of the Laurasiatheria superorder, wherein said member of the Laurasiatheria super order is an ungulate. An ungulate can be a pig.

In some cases, protein expression of said two or more genes can be absent in a genetically modified animal. In some cases, reduction of protein expression inactivates a function of said two or more genes. In some cases, a genetically modified animal can have reduced protein expression of three or more genes. A genetically modified animal can have reduced protein expression of a component of a MHC I-specific enhanceosome, wherein a component of a MHC I-specific enhanceosome can be a NOD-like receptor family CARD domain containing 5 (NLRC5). A genetically modified animal can comprise reduced protein expression of a transporter of a MHC I-binding peptide, wherein a transporter can be a transporter associated with antigen processing 1 (TAP1).

In some cases, a genetically modified animal can comprise reduced protein expression of C3. In some cases, a reduction of protein expression can inactivate a function of two or more genes. In some cases, a reduced protein expression of a NK group 2D ligand can be an MHC class I polypeptide-related sequence A (MICA) or MHC class I polypeptide-related sequence B (MICB). In some cases, reduced protein expression of an endogenous gene may not be expressed in a human, wherein said endogenous gene may not be expressed in a human can be glycoprotein galactosyltransferase alpha 1,3 (GGTA1), putative cytidine monophosphate-N-acetylneuraminic acid hydroxylase-like protein (CMAH), or β1,4 N-acetylgalactosaminyltransferase (B4GALNT2).

In some genetically modified animals described herein, is at least two genomic disruptions resulting in a reduced protein expression of a CXCR3 ligand, which can be C—X—C motif chemokine 10 (CXCL 10).

Provided herein is at least one genetically modified animal further comprising one or more exogenous transgenes encoding at least one protein or functional fragment thereof, wherein said at least one protein is selected from an MHC I formation suppressor, a regulator of complement activation, an inhibitory ligand for NK cells, a B7 family member, CD47, a serine protease inhibitor, galectin, and any combination thereof.

In some cases, the at least one protein can be at least one human protein. One or more exogenous transgenes encoding an MHC I formation suppressor can be infected cell protein 47 (ICP47). In some cases, one or more exogenous transgenes encoding a regulator of complement activation can be cluster of differentiation 46 (CD46), cluster of differentiation 55 (CD55), or cluster of differentiation 59 (CD59). In some cases, one or more exogenous transgenes encoding an inhibitory ligand for NK cells can be leukocyte antigen E (HLA-E), human leukocyte antigen G (HLA-G), or β-2-microglobulin (B2M). In other cases, one or more exogenous transgenes encoding HLA-G, wherein HLA-G can be HLA-G1, HLA-G2, HLA-G3, HLA-G4, HLA-G5, HLA-G6, or HLA-G7. In some cases, HLA-G can be HLA-G1.

In some genetically modified animal provided herein, are provided one or more exogenous transgenes encoding a B7 family member, wherein a B7 family member can be a programmed death-ligand. A programmed death-ligand can be programmed death-ligand 1 (PD-L1) or programmed death-ligand 2 (PD-L2). In some cases, one or more exogenous transgenes can encode both PD-L1 and PD-L2. In some cases, one or more exogenous transgenes can encode a serine protease inhibitor, wherein the serine protease inhibitor can be serine protease inhibitor 9 (Spi9). In some cases, one or more exogenous transgenes can encode a galectin, wherein the galectin can be galectin-9. In some cases, one or more exogenous transgenes can be inserted adjacent to a ubiquitous promoter. A ubiquitous promoter can be a Rosa26 promoter.

In some cases, one or more exogenous transgenes can be inserted adjacent to a promoter of a targeted gene, within said targeted gene, or adjacent to a protospacer adjacent motif (PAM) sequence. In some cases, protein expression of two or more genes can be reduced using a CRISPR/Cas system.

Provided herein is a genetically modified animal having a genomic disruption in at least one gene selected from a group consisting of a component of an MHC I-specific enhanceosome, a transporter of an MHC I-binding peptide, a natural killer (NK) group 2D ligand, a CXC chemokine receptor (CXCR) 3 ligand, MHC II transactivator (CIITA), C3, an endogenous gene not expressed in a human, and any combination thereof, wherein said genetically modified animal has reduced expression of said gene in comparison to a non-genetically modified counterpart animal and said genetically modified animal survives at least 22 days after birth. In some cases, a genetically modified animal can survive at least 23 days, 30 days, 35 days, 50 days, 70 days, 100 days, 150 days, 200 days, 250 days, 300 days, 350 days or 400 days after birth.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 demonstrates an immunotherapeutic strategy centered on the use of genetically modified cell and organ grafts lacking functional expression of MHC class I. The need for maintenance immunosuppression required for the prevention of graft rejection is progressively reduced (or the applicability of transplantation of cell and organ xenografts and the transplantation of stem cell-derived cellular allografts and xenografts is progressively increased) when the transplantation of genetically modified cells and organs is combined with transient use of antagonistic anti-CD40 antibodies and even more when combined with the administration of tolerizing vaccines comprising apoptotic donor cells under the cover of anti-CD40 antibodies.

FIG. 2 demonstrates one strategy of making genetically modified pig islet cells and tolerizing vaccines. Two clonal populations of pigs are created. One population having at least GGTA1 knocked out can be used to create a tolerizing vaccine. The other clonal population of pigs that have at least GGTA1 and MHC I genes (e.g., NRLC5) knocked out can be used for cell, tissues, and/or organ donors.

FIG. 3 demonstrates use of positive and tolerizing vaccines (also referred to as a negative vaccine).

FIG. 4 demonstrates an exemplary approach to extending the survival of xenografts in a subject with infusion of apoptotic donor splenocytes for tolerizing vaccination under the cover of transient immunosuppression.

FIG. 5 shows an exemplary approach to preventing rejection or extending survival of xenografts in a recipient in the absence of chronic and generalized immunosuppression of the xenograft recipient. This exemplary approach includes and integrates three components: i) genetically engineered islets with deficient and/or reduced expression of αGal, MHC class I, complement C3, and CXCL10 and transgenic expression of HLA-G; ii) genetically engineered donor apoptotic and non-apoptotic mononuclear cells (e.g., splenocytes) with deficient and/or reduced expression of αGal, Neu5Gc, and Sda/CAD as well as transgenic expression of HLA-G with or without human CD47, human PD-L1, human PD-L2 (e.g., the genetically engineered vaccine); and iii) the administration of transient immunosuppression including antagonistic anti-CD40 mAb, anti-CD20 mAb, rapamycin, and transient anti-inflammatory therapy including compstatin (e.g., the compstatin derivative APL-2), anti-IL-6 receptor mAb, and soluble TNF receptor.

FIG. 6 demonstrates an exemplary protocol for transplant rejection prophylaxis in a pig-to-cynomolgus monkey islet xenotransplantation. IE: islet equivalent; sTNFR: soluble TNF receptor (e.g., etanercept); α-IL-6R: anti-interleukin 6 receptor; Tx′d: transplanted.

FIGS. 7A-7E demonstrate a strategy for cloning a px330-Gal2-1 plasmid targeting GGTA1. FIG. 7A shows a cloning strategy and oligonucleotides (SEQ ID NOs: 266-267, respectively, in order of appearance) for making a guide RNA targeting GGTA1. FIG. 7B shows an insertion site on the px330 plasmid (SEQ ID NO: 268). FIG. 7C shows a flow chart demonstrating the cloning and verification strategy. FIG. 7D shows a cloning site (SEQ ID NO: 270) and sequencing primers (SEQ ID NOs: 269 and 271, respectively, in order of appearance). FIG. 7E shows sequencing results (SEQ ID NOs: 272-274 respectively, in order of appearance).

FIGS. 8A-8E demonstrate a strategy for cloning a px330-CM1F plasmid targeting CMAH. FIG. 8A shows a cloning strategy and oligonucleotides (SEQ ID NOs: 275 and 276, respectively, in order of appearance) for making a guide RNA targeting CMAH1. FIG. 8B shows an insertion site on the px330 plasmid (SEQ ID NO: 277). FIG. 8C shows a flow chart demonstrating the cloning and verification strategy. FIG. 8D shows a cloning site (SEQ ID NO: 279) and sequencing primers (SEQ ID NOs: 278 and 280, respectively, in order of appearance). FIG. 8E shows sequencing results (SEQ ID NOs: 281-283, respectively, in order of appearance).

FIGS. 9A-9E demonstrate a strategy for cloning a px330-NL1 FIRST plasmid targeting NLRC5. FIG. 9A shows a cloning strategy and oligonucleotides (SEQ ID NOs: 284 and 285, respectively, in order of appearance) for making a guide RNA targeting NLRC5. FIG. 9B shows an insertion site on the px330 plasmid (SEQ ID NO: 286). FIG. 9C shows a flow chart demonstrating the cloning and verification strategy. FIG. 9D shows a cloning site (SEQ ID NO: 288) and sequencing primers (SEQ ID NOs: 287 and 289, respectively, in order of appearance). FIG. 9E shows sequencing results (SEQ ID NOs: 290-292, respectively, in order of appearance).

FIGS. 10A-10E demonstrate a strategy for cloning a px330/C3-5 plasmid targeting C3. FIG. 10A shows a cloning strategy and oligonucleotides (SEQ ID NOs: 293 and 294, respectively, in order of appearance) for making a guide RNA targeting C3. FIG. 10B shows an insertion site on the px330 plasmid (SEQ ID NO: 295). FIG. 10C shows a flow chart demonstrating the cloning and verification strategy. FIG. 10D shows a cloning site (SEQ ID NO: 297) and sequencing primers (SEQ ID NOs: 296 and 298, respectively, in order of appearance). FIG. 10E shows sequencing results (SEQ ID NOs: 299-301, respectively, in order of appearance).

FIGS. 11A-11E demonstrate a strategy for cloning a px330/B41_second plasmid targeting B4GALNT2. FIG. 11A shows a cloning strategy and oligonucleotides (SEQ ID NOs: 302 and 303, respectively, in order of appearance) for making a guide RNA targeting B4GALNT2. FIG. 11B shows an insertion site on the px330 plasmid (SEQ ID NO: 304). FIG. 11C shows a flow chart demonstrating the cloning and verification strategy. FIG. 11D shows a cloning site (SEQ ID NO: 306) and sequencing primers (SEQ ID NOs: 305 and 307, respectively, in order of appearance). FIG. 11E shows sequencing results (SEQ ID NOs: 308-310, respectively, in order of appearance).

FIG. 12 demonstrates a map of Rosa26 locus sequenced in Example 2.

FIGS. 13A-13E demonstrates a strategy for cloning a px330/Rosa exon 1 plasmid targeting Rosa26. FIG. 13A shows a cloning strategy and oligonucleotides (SEQ ID NOs: 311-312, respectively, in order of appearance) for making a guide RNA targeting Rosa26. FIG. 13B shows an insertion site on the px330 plasmid (SEQ ID NO: 313). FIG. 13C shows a flow chart demonstrating the cloning and verification strategy. FIG. 13D shows a cloning site (SEQ ID NO: 315) and sequencing primers (SEQ ID NOs: 314 and 316, respectively, in order of appearance). FIG. 13E shows sequencing results (SEQ ID NOs: 317-319, respectively, in order of appearance).

FIG. 14A shows a map of the genomic sequence of HLA-G. FIG. 14B shows a map of the cDNA sequence of HLA-G.

FIG. 15 shows an exemplary microscopic view of porcine fetal fibroblasts transfected with pSpCas9(BB)-2A-GFP.

FIG. 16 shows a fluorescence in situ hybridization (FISH) to the GGTA1 gene by specific probes revealing the location on chromosome 1.

FIGS. 17A-17B demonstrate an example of phenotypic selection of cells with cas9/sgRNA-mediated GGTA1/NLCR5 disruption. FIG. 17A shows genetically modified cells, which do not express alpha-galactosidase. FIG. 17B shows non-genetically modified cells, which express alpha-galactosidase and were labeled with isolectin B4 (IB)-linked ferrous beads.

FIGS. 18A-18B show sequencing of DNA isolated from fetal cells of two separate litters (Pregnancy 1: FIG. 18A or Pregnancy 2: FIG. 18B) subjected to PCR amplification of the GGTA1 (compared to Sus scrofa breed mixed chromosome 1, Sscrofa10.2 NCBI Reference Sequence: NC_010443.4) target regions and the resulting amplicons were separated on 1% agarose gels. Amplicons were also analyzed by Sanger sequencing using the forward primer alone from each reaction. In FIG. 18A, the results are shown, aligned to reference and target gene sequences (SEQ ID NOs: 320-321, respectively), for fetuses 1-7 (SEQ ID NOs: 322-328, respectively) from Pregnancy 1's fetuses. Fetuses 1, 2, 4, 5, 6, and 7, were truncated 6 nucleotides after the target site for GGTA1. Fetus 3 was truncated 17 nucleotides after the cut site followed by a 2,511 (668-3179) nucleotide deletion followed by a single base substitution. Truncation, deletion and substitution from a single sequencing experiment containing the alleles from both copies of the target gene can only suggest a gene modification has occurred but not reveal the exact sequence for each allele. From this analysis it appears that all 7 fetuses have a single allele modification for GGTA1. In FIG. 18B, the results are shown, aligned to reference and target gene sequences (SEQ ID NOs: 329-330, respectively), for fetuses 1-5 (SEQ ID NOs: 331-335, respectively) from pregnancy 2 fetal DNA samples. Fetuses 1, 3, 4, and 5 were truncated 3 nucleotides from the GGTA1 gene target site. Fetus 2 had variability in Sanger sequencing that suggests a complex variability in DNA mutations or poor sample quality. However, fetal DNA template quality was sufficient for the generation of the GGTA1 gene screening experiment described above.

FIGS. 19A-19B show sequencing of DNA isolated from fetal cells of two separate litters (Pregnancy 1: FIG. 19A or Pregnancy 2: FIG. 19B) subjected to PCR amplification of the NLRC5 (consensus sequence) target regions and the resulting amplicons were separated on 1% agarose gels. Amplicons were also analyzed by Sanger sequencing using the forward primer alone from each reaction. In FIG. 19A, the results are shown, aligned to reference and target gene sequences (SEQ ID NOs: 336-337, respectively) for fetuses 1, 3, 5, 6, and 7 (SEQ ID NOs: 338-342, respectively) from Pregnancy 1. Sequence analysis of the NLRC5 target site was unable to show consistent alignment suggesting an unknown complication in the sequencing reaction or varying DNA modifications between NLRC5 alleles that complicate the Sanger sequencing reaction and analysis. In FIG. 19B, the results are shown, aligned to reference and target gene sequences (SEQ ID NOs: 343-344, respectively) for fetuses 1-5 (SEQ ID NOs: 345-349, respectively) from Pregnancy 2. NLRC5 gene amplicons for fetuses 1-5 were all truncated 120 nucleotides downstream of the NLRC5 gene cut site.

FIGS. 20A-20B show data from fetal DNA (wt and 1-7 (FIG. 20A: Pregnancy 1) or 1-5 (FIG. 20B: Pregnancy 2) isolated from hind limb biopsies. Target genes were amplified by PCR and PCR products were separated on 1% agarose gels and visualized by fluorescent DNA stain. The amplicon band present in the wt lanes represent the unmodified DNA sequence. An increase or decrease in size of the amplicon suggests an insertion or deletion within the amplicon, respectively. Variation in the DNA modification between alleles in one sample may make the band appear more diffuse. Pregnancy 1 (FIG. 20A) resulted in 7 fetuses while pregnancy 2 (FIG. 20B) resulted in 5 fetuses harvested at 45 and 43 days, respectively. A lack of band as in the NLRC5 gel in fetuses 1, 3, and 4 of FIG. 20A (bottom gel), suggests that the modification to the target region have disrupted the binding of DNA amplification primers. The presence of all bands in GGTA 1 in FIG. 20A (top gel) suggests that DNA quality was sufficient to generate DNA amplicons in the NLRC5 targeting PCR reactions. Fetuses 1, 2, 4, and 5 of Pregnancy 1 (FIG. 20A) have larger GGTA 1 amplicons than the WT suggesting an insertion within the target area. In fetus 3 of Pregnancy 1 (FIG. 20A), the GGTA 1 amplicon migrated faster than the WT control suggesting a deletion within the target area. Fetuses 6 and 7 of Pregnancy 1 (FIG. 20A) NLRC5 amplicons migrated faster than the WT suggesting a deletion within the target area. Fetuses 1-5 (FIG. 20B) GGTA1 amplicons were difficult to interpret by size and were diffuse as compared to the WT control. Fetuses 1-5 (FIG. 20B) NLRC5 amplicons were uniform in size and density as compared to the wild type control.

FIGS. 21A-21E shows phenotypic analysis of fetuses from two separate litters of pigs (FIGS. 21A, 21B, 21C: Pregnancy 1 or FIGS. 21D-21E: Pregnancy 2). Fetuses were harvested at day 45 (Pregnancy 1) or 43 days (Pregnancy 2) and processed for DNA and culture cell isolation. Tissue fragments and cells were plated in culture media for 2 days to allow fetal cells to adhere and grow. Wild type cells (fetal cells not genetically modified) and fetal cells from pregnancy 1 and 2 were removed from culture plates and labeled with IB4 lectin conjugated to Alexa fluor 488 or anti-porcine MHC class I antibody conjugated to FITC. Flow cytometric analysis is shown as histograms depicting the labeling intensity of the cells tested. The histograms for the WT cells are included in each panel to highlight the decrease in overall intensity of each group of fetal cells. There is a decrease in alpha Gal and MHC class I labeling in pregnancy 1 (FIG. 21A) indicated as a decrease in peak intensity. In pregnancy 2 (FIG. 21B) fetuses 1 and 3 have a large decrease in alpha gal labeling and significant reduction in MHC class 1 labeling as compared to WT fetal cells.

FIGS. 22A-22C show the impact of decreased MHC class I expression in cells from Fetus 3 (Pregnancy 1) as compared to wild type fetal cells from a genetic clone. The proliferative response of human CD8+ cells and CD4 T cells to porcine control fibroblast and NLRC5 knockout fetal cells were measured. FIG. 22A. Cells were gated as CD4 or CD8 before assessment of proliferation. FIG. 22B. CD8 T cell proliferation was reduced following treatments stimulation by porcine fetal GGTA1/NLRC5 knockout cells compared to control unmodified porcine fibroblast. Almost a 55% reduction in CD8 T cells proliferation was observed when human responders were treated with porcine fetal GGTA1/NLRC5 knockout cells at 1:1 ratio. Wild type fetal cells elicited a 17.2% proliferation in human CD8 T cells whereas the MHC class I deficient cells from fetus 3 (Pregnancy 1) induced only a 7.6% proliferation. FIG. 22C. No differences were seen in CD8 T cells proliferative response at 1:5 and 1:10 ratio compared to unmodified fetal cells. No changes were observed in CD4 T cell proliferation in response to NLRC5 knockout and control unmodified porcine fetal cells at all ratios studied.

FIG. 23 shows live birth of GGTA1/NLRC5 knockout piglets generated using CRISPR/Cas technology.

FIGS. 24A-24C show DNA gel analysis of the genotypes of the piglets generated in Example 6. FIG. 24A shows the result of the first PCR experiment in Example 6. FIG. 24B shows the result of the second PCR experiment in Example 6. FIG. 24C shows the result of the third PCR experiment in Example 6.

FIG. 25A shows the sequencing data and sequence call (SEQ ID NO: 350) of part of NLRC5 gene of piglet #1. FIG. 25B shows the sequencing data and sequence call (SEQ ID NO: 351) of part of NLRC5 gene of piglet #2. FIG. 25C shows the sequencing data and sequence call (SEQ ID NO: 352) of part of NLRC5 gene of piglet #4. FIG. 25D shows the sequencing data and sequence call (SEQ ID NO: 353) of part of NLRC5 gene of piglet #5. FIG. 25E shows the sequencing data and sequence call (SEQ ID NO: 354) of part of NLRC5 gene of piglet #6. FIG. 25F shows the sequencing data and sequence call (SEQ ID NO: 355) of part of NLRC5 gene of piglet #7.

FIG. 26A shows the left arm of Rosa26 in Example 8 (SEQ ID NO: 356). FIG. 26B shows DNA gel analysis of the construct for homology recombination in Example 8. FIG. 26C shows the consensus sequence of amplicon based on paired read analysis in Example 8 (SEQ ID NO: 357). FIGS. 26D (SEQ ID NO: 358), 26E (SEQ ID NO: 359), and 26F (SEQ ID NO: 360) show homology directed recombination construct for inserting HLA-G1 at Rosa26 locus in Example 8.

FIG. 27A shows the sequence of the correct px330 plasmid (SEQ ID NO: 362) containing Rosa26 targeting oligo generated in Example 8, and sequencing primers (SEQ ID NOs: 361 and 363, respectively, in order of appearance). FIG. 27B shows the sequencing result of constructed px330 plasmid containing Rosa26 targeting oligo in Example 8. SEQ ID NOs: 364-366 are disclosed, respectively, in order of appearance. FIG. 27C shows restriction digestion of the constructed px330 plasmid containing Rosa26 targeting oligo in Example 8.

FIG. 28 shows the map of GalMet plasmid and oligos (SEQ ID NOs: 367-368, respectively, in order of appearance) used in Example 8.

FIG. 29 shows in vitro Cas9-mediated cleavage reactions of in vitro transcribed gRNA. Lane 1: Uncleaved pig Rosa26 (2000 bp). Lane 2: designed gRNA directed Cas9 cleavage of pig Rosa26; Lane 3: Uncleaved Pig GGTA1; Lane 4: designed gRNA directed Cas9 cleavage of GGTA1 template.

FIG. 30 shows sorting of genetically modified cell generated in Example 8 by flow cytometry.

FIG. 31 shows the construct for homology recombination of CD47 to GGTA1 locus generated in Example 9 (SEQ ID NO: 369).

FIG. 32 shows the sequence of the right arm (FIG. 32A; SEQ ID NO: 370) and the left arm (FIG. 32B; SEQ ID NO: 371) of GGTA1 locus in Example 9.

FIGS. 33A, 33B, and 33C show the sorting of unstained cells in Example 9.

FIGS. 34A, 34B, and 34C show the sorting of px330 stained cells in Example 9.

FIGS. 35A, 35B, and 35C show the sorting IB4 stained cells in Example 9.

FIGS. 36A, 36B, and 36C show the sorting of CD47/IB4 stained cells in Example 9.

FIGS. 37A, 37B, and 37C show the sorted IB4 stained cells CD47/IB4 stained cells in Example 9.

FIGS. 38A, 38B, and 38C show the sorted CD47/IB4 stained cells in Example 9.

FIG. 39 shows the gating strategy used for the selection of single cells and live cells for analysis. Total CD3+ cells were observed with in that population CD4+ and CD8+ cells were selected and counted for experimental parameters.

FIGS. 40A and 40B show A. unstimulated cells in quadrant 2 showed insignificant expansion when in culture conditions identical to the same cells stimulated with PHA. B. PHA stimulation induced 20.7% (CD3), 24.7% (CD4), 18.4% (CD8), and 21% (CD20) proliferation in lymphocytic samples suggesting the maximum amount of stimulation possible in this assay.

FIG. 41 shows flow cytometry results of a co-culture assay where CD8+ T cells were added to cultures of adherent WT or genetically engineered porcine fibroblasts at a dilution of 100:1, 50:1, 10:1, or 1:1. WT cells stimulated T cells to proliferate at 50:1, 10:1, and 1:1 ratios. GM cells #3 and #4 showed little effect at stimulating T cells at the 100:1, 50:1, and 10:1 ratios suggesting a complete abrogation of T cells proliferation response.

FIG. 42 shows flow cytometry results of a co-culture assay where CD4+ T cells were added to cultures of adherent WT or genetically engineered porcine fibroblasts at a dilution of 100:1, 50:1, 10:1, and 1:1. GM cells #3 and #4 showed little effect at stimulating T cells at the 100:1, 50:1, and 10:1 ratios suggesting a complete abrogation of T cells proliferation response.

FIG. 43 shows flow cytometry results of a co-culture assay where CD3+ T cells (overall CD 4 and CD8) were added to cultures of adherent WT or genetically engineered porcine fibroblasts at a dilution of 100:1, 50:1, 10:1, and 1:1. GM cells #3 and #4 showed little effect at stimulating T cells at the 100:1, 50:1, and 10:1 ratios suggesting a complete abrogation of T cells proliferation response.

FIG. 44 shows B cell proliferation inhibition by approximately 50% when incubated with GGTA1/NLRC5 knock out cells as compared to wild type cells.

FIG. 45 shows flow cytometry results of a co-culture assay where cytokines were measured by incubating human lymphocytes with WT or GM cells followed by the introduction of brefeldin A to block endocytosis causing the accumulation of the 4 cytokines intracellularly in endosomes. Fixation and permeabilization of the cells allows intracellular measurement of the accumulation of cytokines. Within the CD8 T cell population no IL2 stimulation was observed at 100:1 ratio moderate reductions in CD107a, Perforin and Granzyme were observed at the 100:1 ratio. Perforin and granzyme B double positive cells are significantly inhibited at the 100:1 and 10:1 ratios.

FIG. 46 shows flow cytometry results of a co-culture assay of human lymphocytes with WT or genetically modified porcine fibroblasts at a T cells vs FC ratio=10:1. Within the CD8 T cell population IL2 was stimulated at 10:1 ratio and reduced by approximately 40% in culture with genetically modified porcine cells. CD107a, expression was reduced by approximately 25%. Perforin expression was reduced by approximately 40% and Granzyme was unaffected at this ratio of incubation.

FIG. 47 shows flow cytometry results of a co-culture assay of human lymphocytes with WT or genetically modified porcine fibroblasts at a T cells vs FC ratio=10:1. Within CD3 cells CD107a was reduced by approximately 50%. Perforin and Granzyme B was also reduced after incubation with genetically modified cells and was reflected when compared as double positive cells retreating from quadrant 2.

FIG. 48 shows flow cytometry results of a co-culture assay of human lymphocytes with WT or genetically modified porcine fibroblasts at a T cells vs FC ratio=10:1. CD4+ T cells were activated less in the presence of GM cells to produce cytokines. IL2 expression was reduced by 40%. CD107a was reduced by approx. 50%. Perforin and Granzyme B were reduced by approximately 50% and 30%, respectively.

FIG. 49 shows flow cytometry results of a co-culture assay of human lymphocytes with WT or genetically modified porcine fibroblasts at a T cell vs FC ratio=10:1. Among CD3 cells IFNγ expression was significantly reduced when lymphocytes were cultured with GM pig fibroblasts at a 10:1 ratio. TNFa expression was low in culture with WT cells but reduced when in culture with GM cells. Within this experiment Granzyme B was also dramatically reduced when incubated with GM cells as compared to WT cells.

FIG. 50 shows flow cytometry results of a co-culture assay of human lymphocytes with WT or genetically modified porcine fibroblasts at a T cell vs FC ratio=10:1. Among CD4 cells IFNγ expression was significantly reduced when lymphocytes were cultured with GM pig fibroblasts at a 10:1 ratio. TNFa expression was low in culture with WT cells but reduced when in culture with GM cells. Within this experiment Granzyme B was also dramatically reduced when incubated with GM cells as compared to WT cells.

FIG. 51 shows flow cytometry results of a co-culture assay of human lymphocytes with WT or genetically modified porcine fibroblasts at a T cell vs FC ratio=10:1. Among CD8 cells IFNγ expression was significantly reduced when lymphocytes were cultured with GM pig fibroblasts at a 10:1 ratio. TNFa expression was low in culture with WT cells but reduced when in culture with GM cells. Within this experiment Granzyme B was also dramatically reduced when incubated with GM cells as compared to WT cells.

FIG. 52 shows flow cytometry results of a co-culture assay of human lymphocytes with WT or genetically modified porcine fibroblasts at a T cell vs FC ratio=10:1. NK cells (CD56+) have been shown to be activated in the absence of MHC class I expression on cells. IFNγ (y axis) and Granzyme B (x axis) were expressed in co-culture with WT cells but significantly reduced when in co-culture with GGTA1/NLRC5 knock out cells. No expression or change in TNFa expression was observed with GM cells as compared to WT cells.

FIG. 53 shows Human PBMC incubated with WT pig fibroblasts had a normal background percentage of IL10 expressing CD4 positive T cells (11%). GGTA1/NLRC5 knockout cells labeled #3 and #4 respectively (13.3 and 20.2%) had a marginal effect on IL10 expression. Pig fibroblasts expressing the human inspired HLAG1 protein optimized for expression in pigs induced 60.7% of human CD4+ T cells to produce IL10.

FIG. 54 shows that soluble HLA-G (100 ng/ml) blocks the proliferation of CD8+, CD8− and PBMCs in the culture with WT porcine islet. Q1 and Q2 showing proliferating (CFSE lo) and non-proliferating fractions (CFSE hi) fractions, respectively.

FIG. 55 shows the flow cytometry gating strategy used to analyze CD3, CD4, or CD8 populations for cytokine and effector function molecular analysis of cultured human T cells with genetically modified porcine fibroblasts (HLAG1 expressing), WT, or WT plus PT85 antibody.

FIG. 56 shows cytometry data of the CD4 population co-cultured with wild type pig fibroblasts, WT pig fibroblasts with the PT85 antibody, or HLAG1 expressing pig fibroblasts. Substantial decrease in cytokines levels (IL-2) and effector molecules secretion were observed with PT85 blocking or HLAG1 expressing cells at ratio of 10: and 1:1 of MLR culture. The PT85 blocking antibody was used to determine how much of the observed immune inhibitory effects were due to the NLRC5 knock out (MHC class 1 null) or the GGTA1 knock out. The PT85 antibody mimicked the effect of the NLRC5 knockdown in the presence of normal WT alpha-Gal surface expression. The HLAG1 protein expression on the surface of the cells had a profound inhibitory effect on CD4+ and CD8+ T cells cytokine production as well as effector function.

FIG. 57 shows cytometry data of the CD8 population co-cultured with either wild type pig fibroblasts, WT pig fibroblasts with the PT85 antibody, or HLAG1 expressing pig fibroblasts. Substantial decrease in cytokines levels (IL-2) and effector molecules secretion were observed with PT85 blocking or HLAG1 expressing cells at ratio of 10: and 1:1 of MLR culture. The PT85 blocking antibody was used to determine how much of the observed immune inhibitory effects were due to the NLRC5 knock out (MHC class 1 null) or the GGTA1 knock out. The PT85 antibody mimicked the effect of the NLRC5 knockdown in the presence of normal WT alpha-Gal surface expression within the CD8 population. The HLAG1 protein expression on the surface of the cells had a profound inhibitory effect on CD4+ and CD8+ T cells cytokine production as well as effector functions. The HLAG1 protein expression on the surface of the cells had a profound inhibitory effect on CD4+ and CD8+ T cells cytokine production as well as effector functions.

FIG. 58 shows cytometry data of the CD4 population co-cultured with wild type pig fibroblasts, WT pig fibroblasts with the PT85 antibody, or HLAG1 expressing pig fibroblasts. Substantial decrease in cytokines levels (TNF-a, IFN-g) and effector molecules secretion was observed with PT85 blocking or HLAG1 expressing cells at ratio of 10: and 1:1 of MLR culture. The PT85 blocking antibody was used to determine how much of the observed immune inhibitory effects were due to the NLRC5 knock out (MHC class 1 null) or the GGTA1 knock out. The PT85 antibody mimicked the effect of the NLRC5 knockdown in the presence of normal WT alpha-Gal surface expression. The HLAG1 protein expression on the surface of the cells had a profound inhibitory effect on CD4+ and CD8+ T cells cytokine production as well as effector functions.

FIG. 59 shows cytometry data of the CD8 population co-cultured with wild type pig fibroblasts, WT pig fibroblasts with the PT85 antibody, or HLAG1 expressing pig fibroblasts. Substantial decrease in cytokines levels (TNF-a, IFN-g) and effector molecules secretion was observed with PT85 blocking or HLAG1 expressing cells at ratio of 10: and 1:1 of MLR culture. The PT85 blocking antibody was used to determine how much of the observed immune inhibitory effects were due to the NLRC5 knock out (MHC class 1 null) or the GGTA1 knock out. The PT85 antibody mimicked the effect of the NLRC5 knockdown in the presence of normal WT alpha-Gal surface expression. The HLAG1 protein expression on the surface of the cells had a profound inhibitory effect on CD4+ and CD8+ T cells cytokine production as well as effector functions.

FIG. 60 shows the flow gating scheme for the cellular proliferation/CFSE low population analysis.

FIGS. 61 A and B shows flow cytometric analysis of a cellular proliferation (CFSE dilution) experiment of CD3, CD4, or CD8 populations among A. unstimulated cells or B. PHA stimulated cells (positive control or maximal dilution).

FIG. 62 shows that T cell proliferation was reduced following stimulation by porcine fibroblast treated with PT-85 blocking Abs compared to control unmodified porcine fibroblast/WT at ratios 10:1 of Human PBMCs and FC respectively. Substantial reduction in T cells (CD3) proliferation was observed when human responder were treated with SLA-I blocking PT-85 Abs or HLA-G expressing at 10:1 and 1:1 ratio. Not much difference was seen in T cells proliferative response at 100:1 and 50:1 ratio compared to unmodified/WT porcine fibroblast.

FIG. 63 shows that T cell proliferation was reduced following stimulation by porcine fibroblast treated with PT-85 blocking Abs compared to control unmodified porcine fibroblast/WT at ratios 10:1 of Human PBMCs and FC respectively. Substantial reduction in T cells (CD4) proliferation was observed when human responder were treated with SLA-I blocking PT-85 Abs or HLA-G expressing at 10:1 and 1:1 ratio. Not much difference was seen in T cells proliferative response at 100:1 and 50:1 ratio compared to unmodified/WT porcine fibroblast.

FIG. 64 shows reduced T cell proliferation following stimulation by porcine fibroblast treated with PT-85 blocking Abs compared to control unmodified porcine fibroblast/WT at ratios 10:1 of Human PBMCs and FC respectively. Substantial reduction in T cells (CD8) proliferation was observed when human responder were treated with SLA-I blocking PT-85 Abs or HLA-G expressing at 10:1 and 1:1 ratio. Not much difference was seen in T cells proliferative response at 100:1 and 50:1 ratio compared to unmodified/WT porcine fibroblast.

FIG. 65 shows reduced T cell proliferation following stimulation by porcine fibroblast treated with PT-85 blocking Abs compared to control unmodified porcine fibroblast/WT at ratios 10:1 of Human PBMCs and FC respectively. No substantial reduction in B cells proliferation either with blocking SLA-I with PT-85 or HLA-G expression.

FIG. 66 shows that IFNγ is produced predominantly by natural killer (NK) and natural killer T (NKT) cells as part of the innate immune response. DKO #3 and #4 are genetically and phenotypically GGTA1/NLRC5 knock out cells made separately. IFNγ is also produced by CD4 Th1 and CD8 cytotoxic T lymphocyte (CTL) effector T cells after antigen-specific immunity develops.

FIG. 67 shows GMC-SF production among genetically modified cells cultured with human immune cells and controls. Double knock out (DKO) cells had no ability to stimulate GM-CSF production. HLAG1 had significantly reduced expression. DKO #3 and #4 are genetically and phenotypically GGTA1/NLRC5 knock out cells made separately.

FIG. 68 shows IL-17 A expression among genetically modified cells cultured with human immune cells. DKO and HLAG1 transgenic cells both had no ability to induce a pro inflammatory response from human PBMC.

FIG. 69 shows Fractalkine expression among genetically modified porcine cells cultured with human immune cells. HLAG1 expression remains a significant inhibitor of T cells activation and fractalkine production though expressed on a log scale.

FIG. 70 shows TNF alpha expression among genetically modified porcine cells cultured with human immune cells.

FIG. 71 shows the IL-6 production among genetically modified porcine cells cultured with human immune cells.

FIG. 72 shows IL-4 production among genetically modified porcine cells cultured with human immune cells.

FIG. 73 shows MIP 1 alpha production among genetically modified porcine cells cultured with human immune cells.

FIG. 74 shows MIP 1 beta production among genetically modified porcine cells cultured with human immune cells.

FIG. 75 shows that T cell proliferation was reduced following stimulation by porcine fibroblast treated with PT-85 blocking Abs compared to control unmodified porcine fibroblast/WT at ratios 10:1 of Human PBMCs and FC respectively. Substantial reduction in T cells (CD3) proliferation was observed when human responder were treated with SLA-I blocking PT-85 Abs or HLA-G expressing at 10:1 and 1:1 ratio. Not much difference was seen in T cells proliferative response at 100:1 and 50:1 ratio compared to unmodified/WT porcine fibroblast.

FIG. 76 shows that T cell proliferation was reduced following stimulation by porcine fibroblast treated with PT-85 blocking Abs compared to control unmodified porcine fibroblast/WT at ratios 10:1 of Human PBMCs and FC respectively. Substantial reduction in T cells (CD4) proliferation was observed when human responder were treated with SLA-I blocking PT-85 Abs or HLA-G expressing at 10:1 and 1:1 ratio. Not much difference was seen in T cells proliferative response at 100:1 and 50:1 ratio compared to unmodified/WT porcine fibroblast.

FIG. 77 shows reduced T cell proliferation following stimulation by porcine fibroblast treated with PT-85 blocking Abs compared to control unmodified porcine fibroblast/WT at ratios 10:1 of Human PBMCs and FC respectively. Substantial reduction in T cells (CD8) proliferation was observed when human responder were treated with SLA-I blocking PT-85 Abs or HLA-G expressing at 10:1 and 1:1 ratio. Not much difference was seen in T cells proliferative response at 100:1 and 50:1 ratio compared to unmodified/WT porcine fibroblast.

FIG. 78 shows reduced T cell proliferation following stimulation by porcine fibroblast treated with PT-85 blocking Abs compared to control unmodified porcine fibroblast/WT at ratios 10:1 of Human PBMCs and FC respectively. No substantial reduction in B cells proliferation either with blocking SLA-I with PT-85 or HLA-G expression

FIG. 79 shows IFN gamma expression after co-culture of human mixed lymphocytes and porcine genetically modified cells. Double knock out (DKO) #3 and #4 are genetically and phenotypically GGTA1/NLRC5 knock out cells made separately. The HLA-G1 transgenic cells were conducted in a separate experiment with human donor #2 (FIG. 79B) (but not human donor #1; FIG. 79A), and therefore include matching unstimulated and wild type cell controls.

FIG. 80 shows GM-CSF gamma expression after co-culture of human mixed lymphocytes and porcine genetically modified cells. Double knock out (DKO) #3 and #4 are genetically and phenotypically GGTA1/NLRC5 knock out cells made separately. The HLA-G1 transgenic cells were conducted in a separate experiment with human donor #2 (FIG. 80B) (but not human donor #1; FIG. 80A), and therefore include matching unstimulated and wild type cell controls.

FIG. 81 shows IL-2 expression after co-culture of human donor #1 mixed lymphocytes and porcine genetically modified cells. Double knock out (DKO) #3 and #4 are genetically and phenotypically GGTA1/NLRC5 knock out cells made separately.

FIG. 82 shows IL-17 alpha expression after co-culture of human mixed lymphocytes from two donors (FIGS. 82A and B) and porcine genetically modified cells. Double knock out (DKO) #3 and #4 are genetically and phenotypically GGTA1/NLRC5 knock out cells made separately. DKO and HLA-G1 transgenic cells both had no ability to induce a pro inflammatory response from human PBMC.

FIG. 83 shows Fractalkine expression after co-culture of human mixed lymphocytes and porcine genetically modified cells. Double knock out (DKO) #3 and #4 are genetically and phenotypically GGTA1/NLRC5 knock out cells made separately. The HLA-G1 transgenic cells were conducted in a separate experiment with human donor #2 (FIG. 83B) (but not human donor #1; FIG. 83A), and therefore include matching unstimulated and wild type cell controls. HLA-G1 expression remains a significant inhibitor of T cells activation and fractalkine production though expressed on a log scale.

FIG. 84 shows TNF alpha expression after co-culture of human mixed lymphocytes and porcine genetically modified cells. Double knock out (DKO) #3 and #4 are genetically and phenotypically GGTA1/NLRC5 knock out cells made separately. The HLA-G1 transgenic cells were conducted in a separate experiment with human donor #2 (FIG. 84B) (but not human donor #1; FIG. 84A), and therefore include matching unstimulated and wild type cell controls.

FIG. 85 shows IL-6 expression after co-culture of human mixed lymphocytes and porcine genetically modified cells. Double knock out (DKO) #3 and #4 are genetically and phenotypically GGTA1/NLRC5 knock out cells made separately. The HLA-G1 transgenic cells were conducted in a separate experiment with human donor #2 (FIG. 85B) (but not human donor #1; FIG. 85A), and therefore include matching unstimulated and wild type cell controls.

FIG. 86 shows IL-4 expression after co-culture of human mixed lymphocytes and porcine genetically modified cells. Double knock out (DKO) #3 and #4 are genetically and phenotypically GGTA1/NLRC5 knock out cells made separately. The HLA-G1 transgenic cells were conducted in a separate experiment with human donor #2 (FIG. 86B) (but not human donor #1; FIG. 86A), and therefore include matching unstimulated and wild type cell controls.

FIG. 87 shows MIP-1 alpha expression after co-culture of human mixed lymphocytes and porcine genetically modified cells. Double knock out (DKO) #3 and #4 are genetically and phenotypically GGTA1/NLRC5 knock out cells made separately. The HLA-G1 transgenic cells were conducted in a separate experiment with human donor #2 (FIG. 87B) (but not human donor #1; FIG. 87A), and therefore include matching unstimulated and wild type cell controls.

FIG. 88 shows MIP-1 beta expression after co-culture of human mixed lymphocytes and porcine genetically modified cells. Double knock out (DKO) #3 and #4 are genetically and phenotypically GGTA1/NLRC5 knock out cells made separately. The HLA-G1 transgenic cells were conducted in a separate experiment with human donor #2 (FIG. 88B) (but not human donor #1; FIG. 88A), and therefore include matching unstimulated and wild type cell controls.

FIG. 89 shows the CRISPR/Cas construct within the PX333 vector.

FIG. 90 shows transfection schematic of primary porcine fibroblast using constructs: GGTA1-10/B4GALNT2 (condition 2), NLRC5-6/B4GALNT2 (condition 3), GGTA1-10/B4GALNT2 and NLRC5-6/B4GALNT2 (condition 4), Condition 1 (WT): cells only.

FIG. 91 shows genetically Modified Selection using Magnetic Bead Sorting.

FIG. 92 shows Genetically Modified Selection using Cell Sort of SLA I+/IB4+(top right); SLA I+/IB4− (bottom right); SLA I−/IB4+(top left); and SLA I−/IB4 (bottom left).

FIG. 93 shows flow cytometric analysis of Condition 2: GGTA1-10/B4GALNT2.

FIG. 94 shows flow cytometric analysis of Condition 3: NLRC5-6/B4GALNT2.

FIG. 95 shows flow cytometric analysis of Condition 4: GGTA1-10/B4GALNT2+NLRC5-6/B4GALNT2.

FIG. 96 shows flow cytometric analysis of Condition 2: GGTA1-10/B4GALNT2 post sort. Each population was sorted to verify that the right population was acquired post sorting and there was no cross-samples from other gates.

FIG. 97 shows flow cytometric analysis of Condition 3: NLRC5-6/B4GALNT2. Each population was sorted to verify that the right population was acquired post sorting and there was no cross-samples from other gates.

FIG. 98 shows flow cytometric analysis of Condition 4: GGTA1-10/B4GALNT2+NLRC5-6/B4GALNT2. Each population was sorted to verify that the right population was acquired post sorting and there was no cross-samples from other gates.

FIGS. 99A and 99B show flow cytometric analysis of IB4 lectin among A. WT unstained, all cells unstained, WT negative, and condition #2 Gal negative fraction cultured with WT or PFF1. B. Side scatter vs forward scatter of condition #4 Gal negative fraction, WT positive, Conditions #2 Gal Positive fraction. Condition #3Gal positive fraction or conditions #4 Gal positive fraction cultured with WT or PFF1.

FIG. 100 shows flow cytometric quantification of condition 1 (WT), 2, 3, and 4 (left to right, respectively) genetically modified cells.

FIGS. 101 A. and 101 B. shows flow cytometric analysis of SLAI among A. WT unstained, All cells unstained, WT negative, and condition #2 Gal negative fraction cultured with WT or PFF1. B. Side scatter vs forward scatter of condition #4 Gal negative fraction, WT positive, Conditions #2 Gal Positive fraction. Condition #3Gal positive fraction or conditions #4 Gal positive fraction cultured with WT or PFF1.

FIG. 102 shows flow cytometric quantification of SLA1 (FITC) among A. Condition 3 cells and B. Condition 4 cells.

FIGS. 103 A. and 103 B. show confocal microscopy of A. imaging results of WT porcine cells and genetically modified condition 2, 3, and 4 cells. B. slides of imaged produced.

FIG. 104 shows sequencing results of NLRC5 sequencing of condition and condition 4 cell lines. SEQ ID NOs: 372-376 are disclosed, respectively, in order of appearance.

FIG. 105 shows a table of PCR oligos and target sequences (column two) for GG1, Gal2-1, Gal 2-2, Gal 2-3, Gal 2-4, Gal 2-5, GGTA1-10, GGTA1-11, GGTA1-16, NL1, NLRC5-6, NLRC5-7, NLRC5-8. SEQ ID NOs: 377-404 are disclosed, respectively, in order of appearance in column 2. SEQ ID NOs: 405-413 are disclosed, respectively, in order of appearance in column 4. SEQ ID NOs: 414-422 are disclosed, respectively, in order of appearance in column 6.

FIG. 106 shows a table of PCR oligos and target sequences (column two) for CM1F, CM2RS, CM3RS, CM4RS. SEQ ID NOs: 423-430 are disclosed, respectively, in order of appearance in column 2. SEQ ID NOs: 431-434 are disclosed, respectively, in order of appearance in column 4. SEQ ID NOs: 435-437 are disclosed, respectively, in order of appearance in column 6.

FIGS. 107 A. and 107 B. show a table of A. Target sequences for gRNAs for B41, C3-9_1, C3-9_2, C3-5_1, C3-5_2, C3-15RS_1, C3-15RS_2. SEQ ID NOs: 438-447 are disclosed, respectively, in order of appearance in column 2. B. Deletion screening primer sequences and their respective target sequence for Gal 1. SEQ ID NOs: 448-453 are disclosed, respectively, in order of appearance in column 2.

FIG. 108 shows an overview of a Gal2-2 (B4GALNT2) vector and cloning strategy. A nucleotide sequence for a portion of the vector is disclosed (SEQ ID NO: 454), as well as two oligos: Gal2-2_Forward (SEQ ID NO): 455) and Gal2-2_Reverse (SEQ ID NO: 456).

FIG. 109 shows the expected Gal2-2 (B4GALNT2) clone sequence upon correct insertion based on the vector and cloning strategy of FIG. 113 (top panel). SEQ ID NOs: 457-459 are disclosed, respectively, in order of appearance. The sequencing results of the constructed plasmid (SEQ ID NO: 462) are aligned against the expected sequence (SEQ ID NOs: 460-461) in the bottom panel.

FIGS. 110 A. and B. A. shows the Gal2-1 (B4GALNT2) target site within GGTA1 gene (SEQ ID NO: 464) and two oligos (Gal2-1_screen_Forward_1, SEQ ID NO: 463; and Gal2-1_screen_Reverse_1, SEQ ID NO: 465). B. shows Gal2-1_screen_1 primer set, Gal2-1_screen primer set PCR product observed on gel and expected amplicon size of 303 bp. The strong single band observed at expected amplicon size product was sequence verified and was shown to include Gal2-1 target cut-sites desired for screening.

FIGS. 111 A. and 111 B. A. shows the CM1F target site within CMAH gene (SEQ ID NO: 467) and two oligos (CM1F-1_screen_Forward_1, SEQ ID NO: 466; and CM1F-1_screen_Reverse_1, SEQ ID NO: 468). B. shows CM1F_screen_1 primer set expected amplicon size of 309 bp, CM1F_screen primer set PCR product observed on gel. A strong band observed at the expected amplicon size; faint band observed at ˜600 bp as well. Product at approximately 300 bp was sequence verified and was shown to include the target cut-site as desired for screening.

FIGS. 112 A. and 112 B. A. shows NL1_First target site within NLRC5 gene (SEQ ID NO: 470) and two oligos (NLR amp2 forward, SEQ ID NO: 469; and NLR amp2 reverse, SEQ ID NO: 471). B. shows NLR amp 2 primer set expected amplicon size: 217 bp, NLR amp 2 primer set PCR product observed on gel, the strong single band observed at the expected amplicon size. The product was sequence verified and was shown to include NL1_First target cut-site as desired for screening.

FIGS. 113 A to 113 I represent exon 1 genomic modifications of Gal2-2 and NLRC5 genes. A. shows the location of screening primers for Gal. B. Gal2-2 PCR Screen using Gal2-2 screen 1 primers. C. sequence results for Gal 2-2. SEQ ID NOs: 472-478 are disclosed respectively, in order of appearance. D. GGTA1-10 PCR screen using GGTA1-10,11 screen primers. E. NLRC5-6 Screen Primers Location F. NLRC5-6 set A (NLRC5-678 screen primers) G. NLRC5-6 Sequence Results from Set A. SEQ ID NOs: 479-486 are disclosed respectively, in order of appearance. H. NLRC5-6 set B (NLRC5-678 Forward and NLR1st screen 2 Reverse screen primers) I. NLRC5-6 set C (NLRC5-678 Forward and NLR1st screen 2 Reverse screen primers).

FIGS. 114 A-C show live births of GGTA1/NLRC5 knockout/HLA-G1 knockin piglets generated using CRISPR/Cas technology.

FIG. 115 shows the sequencing results confirming insertion of HLA-G1 into the ROSA gene site. SEQ ID NO: 499 is disclosed.

FIG. 116 shows the sequence results confirming correct construction of the homology directed recombination construct for inserting HLA-G1 at Rosa26 locus in Example 8. SEQ ID NO: 500 is disclosed.

FIG. 117 shows the sequence of a left arm corresponding to the Rosa26 locus that can be used in the construction of a homology targeting vector for insertion of HLA-G1, or another sequence, into the Rosa26 locus. SEQ ID NO: 501 is disclosed.

FIG. 118 shows the sequence of a modified HLA-G encoding sequence that can be used in the construction of a homology targeting vector for insertion of HLA-G1 into a genetic loci such as a Rosa26 locus. SEQ ID NO: 502 is disclosed.

FIG. 119 shows the sequence of a right arm corresponding to the Rosa26 locus that can be used in the construction of a homology targeting vector for insertion of HLA-G1, or another sequence, into the Rosa26 locus. SEQ ID NO: 503 is disclosed.

### DETAILED DESCRIPTION OF THE DISCLOSURE

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

Graft rejection can be prevented by methods tempering the immune response, including those described herein. For example, one method described herein to prevent transplantation rejection or prolong the time to transplantation rejection without or with minimal immunosuppressive drug use, an animal, e.g., a donor non-human animal, could be altered, e.g., genetically. Subsequently, the cells, organs, and/or tissues of the altered animal, e.g., a donor non-human animal, can be harvested and used in allografts or xenografts. Alternatively, cells can be extracted from an animal, e.g., a human or non-human animal (including but not limited to primary cells) or cells can be previously extracted animal cells, e.g., cell lines. These cells can be used to create a genetically altered cell.

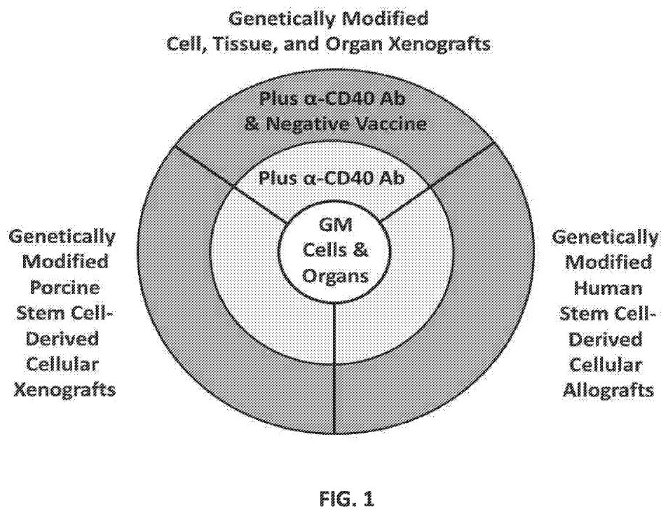

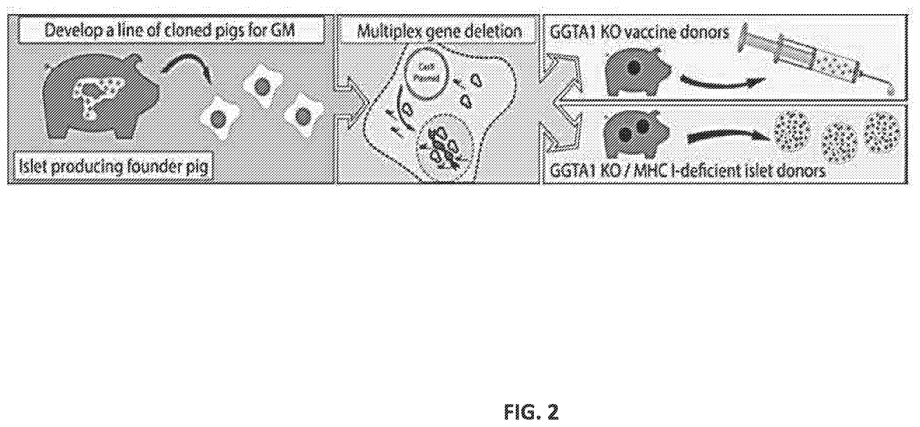

Transplant rejection (e.g., T cells-mediated transplant rejection) can be prevented by chronic immunosuppression. However, immunosuppression is costly and associated with the risk of serious side effects. To circumvent the need for chronic immunosuppression, a multifaceted, T cell-targeted rejection prophylaxis was developed (FIG. 1) that
i) utilizes genetically modified grafts lacking functional expression of MHC class I, thereby interfering with activation of CD8⁺T cells with direct specificity and precluding cytolytic effector functions of these CD8⁺ T cells,
ii) interferes with B cell (and other APC)-mediated priming and memory generation of anti-donor T cells using induction immunotherapy comprising antagonistic anti-CD40 mAbs (and depleting anti-CD20 mAbs and a mTOR inhibitor), and/or
iii) depletes anti-donor T cells with indirect specificity via peritransplant infusions of apoptotic donor cell vaccines.

Described herein are genetically modified non-human animals (such as non-human primates or a genetically modified animal that is member of the Laurasiatheria superorder, e.g., ungulates) and organs, tissues, or cells isolated therefrom, tolerizing vaccines, and methods for treating or preventing a disease in a recipient in need thereof by transplantation of an organ, tissue, or cell isolated from a non-human animal. An organ, tissue, or cell isolated from a non-human animal (such as non-human primates or a genetically modified animal that is member of the Laurasiatheria superorder, e.g., ungulates) can be transplanted into a recipient in need thereof from the same species (an allotransplant) or a different species (a xenotransplant). A recipient can be tolerized with a tolerizing vaccine and/or one or more immunomodulatory agents (e.g., an antibody). In embodiments involving xenotransplantation the recipient can be a human. Suitable diseases that can be treated are any in which an organ, tissue, or cell of a recipient is defective or injured, (e.g., a heart, lung, liver, vein, skin, or pancreatic islet cell) and a recipient can be treated by transplantation of an organ, tissue, or cell isolated from a non-human animal.

Human Leukocyte Antigen G (HLA-G) HLA-G can be a potent immuno-inhibitory and tolerogenic molecule. Accordingly, in one aspect, disclosed herein are genetically modified non-human animals and cells comprising an exogenous nucleic acid sequence encoding for an HLA-G protein. The genetically modified non-human animals and cells can also comprise one or more additional genetic modifications, such as any of the genetic modifications (e.g., knock-ins, knock-outs, gene disruptions, etc.) disclosed herein.

### Definitions

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

The term “non-human animal” and its grammatical equivalents as used herein includes all animal species other than humans, including non-human mammals, which can be a native animal or a genetically modified non-human animal. A non-human mammal includes, an ungulate, such as an even-toed ungulate (e.g., pigs, peccaries, hippopotamuses, camels, llamas, chevrotains (mouse deer), deer, giraffes, pronghorn, antelopes, goat-antelopes (which include sheep, goats and others), or cattle) or an odd-toed ungulate (e.g., horse, tapirs, and rhinoceroses), a non-human primate (e.g., a monkey, or a chimpanzee), a Canidae (e.g., a dog) or a cat. A non-human animal can be a member of the Laurasiatheria superorder. The Laurasiatheria superorder can include a group of mammals as described in Waddell et al., Towards Resolving the Interordinal Relationships of Placental Mammals. Systematic Biology 48 (1): 1-5 (1999). Members of the Laurasiatheria superorder can include Eulipotyphla (hedgehogs, shrews, and moles), Perissodactyla (rhinoceroses, horses, and tapirs), Carnivora (carnivores), Cetartiodactyla (artiodactyls and cetaceans), Chiroptera (bats), and Pholidota (pangolins). A member of Laurasiatheria superorder can be an ungulate described herein, e.g., an odd-toed ungulate or even-toed ungulate. An ungulate can be a pig. A member can be a member of Carnivora, such as a cat, or a dog. In some cases, a member of the Laurasiatheria superorder can be a pig.

The term “pig” and its grammatical equivalents as used herein can refer to an animal in the genus Sus, within the Suidae family of even-toed ungulates. For example, a pig can be a wild pig, a domestic pig, mini pigs, a Sus scrofa pig, a Sus scrofa domesticus pig, or inbred pigs.

The term “transgene” and its grammatical equivalents as used herein can refer to a gene or genetic material that can be transferred into an organism. For example, a transgene can be a stretch or segment of DNA containing a gene that is introduced into an organism. The gene or genetic material can be from a different species. The gene or genetic material can be synthetic. When a transgene is transferred into an organism, the organism can then be referred to as a transgenic organism. A transgene can retain its ability to produce RNA or polypeptides (e.g., proteins) in a transgenic organism. A transgene can comprise a polynucleotide encoding a protein or a fragment (e.g., a functional fragment) thereof. The polynucleotide of a transgene can be an exogenous polynucleotide. A fragment (e.g., a functional fragment) of a protein can comprise at least or at least about 5%, 10%, 20%, 30%, 40%, 50%, 60%, 70%, 80%, 90%, 95%, or 99% of the amino acid sequence of the protein. A fragment of a protein can be a functional fragment of the protein. A functional fragment of a protein can retain part or all of the function of the protein.

The term “exogenous nucleic acid sequence” can refer to a gene or genetic material that was transferred into a cell or animal that originated outside of the cell or animal. An exogenous nucleic acid sequence can by synthetically produced. An exogenous nucleic acid sequence can be from a different species, or a different member of the same species. An exogenous nucleic acid sequence can be another copy of an endogenous nucleic acid sequence.

The term “genetic modification” and its grammatical equivalents as used herein can refer to one or more alterations of a nucleic acid, e.g., the nucleic acid within an organism's genome. For example, genetic modification can refer to alterations, additions, and/or deletion of genes. A genetically modified cell can also refer to a cell with an added, deleted and/or altered gene. A genetically modified cell can be from a genetically modified non-human animal. A genetically modified cell from a genetically modified non-human animal can be a cell isolated from such genetically modified non-human animal. A genetically modified cell from a genetically modified non-human animal can be a cell originated from such genetically modified non-human animal.

The term “gene knock-out” or “knock-out” can refer to any genetic modification that reduces the expression of the gene being “knocked out.” Reduced expression can include no expression. The genetic modification can include a genomic disruption.

The term “islet” or “islet cells” and their grammatical equivalents as used herein can refer to endocrine (e.g., hormone-producing) cells present in the pancreas of an organism. For example, islet cells can comprise different types of cells, including, but not limited to, pancreatic α cells, pancreatic β cells, pancreatic δ cells, pancreatic F cells, and/or pancreatic ε cells. Islet cells can also refer to a group of cells, cell clusters, or the like.

The term “condition” condition and its grammatical equivalents as used herein can refer to a disease, event, or change in health status.

The term “diabetes” and its grammatical equivalents as used herein can refer to is a disease characterized by high blood sugar levels over a prolonged period. For example, the term “diabetes” and its grammatical equivalents as used herein can refer to all or any type of diabetes, including, but not limited to, type 1, type 2, cystic fibrosis-related, surgical, gestational diabetes, and mitochondrial diabetes. In some cases, diabetes can be a form of hereditary diabetes.

The term “phenotype” and its grammatical equivalents as used herein can refer to a composite of an organism's observable characteristics or traits, such as its morphology, development, biochemical or physiological properties, phenology, behavior, and products of behavior. Depending on the context, the term “phenotype” can sometimes refer to a composite of a population's observable characteristics or traits.

The term “disrupting” and its grammatical equivalents as used herein can refer to a process of altering a gene, e.g., by deletion, insertion, mutation, rearrangement, or any combination thereof. For example, a gene can be disrupted by knockout. Disrupting a gene can be partially reducing or completely suppressing expression (e.g., mRNA and/or protein expression) of the gene. Disrupting can also include inhibitory technology, such as shRNA, siRNA, microRNA, dominant negative, or any other means to inhibit functionality or expression of a gene or protein.

The term “gene editing” and its grammatical equivalents as used herein can refer to genetic engineering in which one or more nucleotides are inserted, replaced, or removed from a genome. For example, gene editing can be performed using a nuclease (e.g., a natural-existing nuclease or an artificially engineered nuclease).

The term “transplant rejection” and its grammatical equivalents as used herein can refer to a process or processes by which an immune response of an organ transplant recipient mounts a reaction against the transplanted material (e.g., cells, tissues, and/or organs) sufficient to impair or destroy the function of the transplanted material.

The term “hyperacute rejection” and its grammatical equivalents as used herein can refer to rejection of a transplanted material or tissue occurring or beginning within the first 24 hours after transplantation. For example, hyperacute rejection can encompass but is not limited to “acute humoral rejection” and “antibody-mediated rejection”.

The term “negative vaccine”, “tolerizing vaccine” and their grammatical equivalents as used herein, can be used interchangeably. A tolerizing vaccine can tolerize a recipient to a graft or contribute to tolerization of the recipient to the graft if used under the cover of appropriate immunotherapy. This can help to prevent transplantation rejection.

The term “recipient”, “subject” and their grammatical equivalents as used herein, can be used interchangeably. A recipient or a subject can be a human or non-human animal. A recipient or a subject can be a human or non-human animal that will receive, is receiving, or has received a transplant graft, a tolerizing vaccine, and/or other composition disclosed in the application. A recipient or subject can also be in need of a transplant graft, a tolerizing vaccine and/or other composition disclosed in the application. In some cases, a recipient can be a human or non-human animal that will receive, is receiving, or has received a transplant graft.

Some numerical values disclosed throughout are referred to as, for example, “X is at least or at least about 100; or 200 [or any numerical number].” This numerical value includes the number itself and all of the following:
i) X is at least 100;
ii) X is at least 200;
iii) X is at least about 100; and
iv) X is at least about 200.
All these different combinations are contemplated by the numerical values disclosed throughout. All disclosed numerical values should be interpreted in this manner, whether it refers to an administration of a therapeutic agent or referring to days, months, years, weight, dosage amounts, etc., unless otherwise specifically indicated to the contrary.

The ranges disclosed throughout are sometimes referred to as, for example, “X is administered on or on about day 1 to 2; or 2 to 3 [or any numerical range].” This range includes the numbers themselves (e.g., the endpoints of the range) and all of the following:
i) X being administered on between day 1 and day 2;
ii) X being administered on between day 2 and day 3;
iii) X being administered on between about day 1 and day 2;
iv) X being administered on between about day 2 and day 3;
v) X being administered on between day 1 and about day 2;
vi) X being administered on between day 2 and about day 3;
vii) X being administered on between about day 1 and about day 2; and
viii) X being administered on between about day 2 and about day 3.
All these different combinations are contemplated by the ranges disclosed throughout. All disclosed ranges should be interpreted in this manner, whether it refers to an administration of a therapeutic agent or referring to days, months, years, weight, dosage amounts, etc., unless otherwise specifically indicated to the contrary.

The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.”

The term “or” can be used conjunctively or disjunctively, unless the context specifically refers to a disjunctive use.

I. Genetically Modified Non-Human Animals

Provided herein are genetically modified non-human animals that can be donors of cells, tissues, and/or organs for transplantation. A genetically modified non-human animal can be any desired species. For example, a genetically modified non-human animal described herein can be a genetically modified non-human mammal. A genetically modified non-human mammal can be a genetically modified ungulate, including a genetically modified even-toed ungulate (e.g., pigs, peccaries, hippopotamuses, camels, llamas, chevrotains (mouse deer), deer, giraffes, pronghorn, antelopes, goat-antelopes (which include sheep, goats and others), or cattle) or a genetically modified odd-toed ungulate (e.g., horse, tapirs, and rhinoceroses), a genetically modified non-human primate (e.g., a monkey, or a chimpanzee) or a genetically modified Canidae (e.g., a dog). A genetically modified non-human animal can be a member of the Laurasiatheria superorder. A genetically modified non-human animal can be a non-human primate, e.g., a monkey, or a chimpanzee. If a non-human animal is a pig, the pig can be at least or at least about 1, 5, 50, 100, or 300 pounds, e.g., the pig can be or be about between 5 pounds to 50 pounds; 25 pounds to 100 pounds; or 75 pounds to 300 pounds. In some cases, a non-human animal is a pig that has given birth at least one time.

A genetically modified non-human animal can be of any age. For example, the genetically modified non-human animal can be a fetus; from or from about 1 day to 1 month; from or from about 1 month to 3 months; from or from about 3 months to 6 months; from or from about 6 months to 9 months; from or from about 9 months to 1 year; from or from about 1 year to 2 years. A genetically modified non-human animal can be a non-human fetal animal, perinatal non-human animal, neonatal non-human animal, preweaning non-human animal, young adult non-human animal, or an adult non-human animal.

A genetically modified non-human animal can survive for at least a period of time after birth. For example the genetically modified non-human animal can survive for at least 1 day, 2 days, 3 days, 1 week, 2 week, 3 week, 1 month, 2 months, 4 months, 8 months, 1 year, 2 years, 5 years, or 10 years after birth. Multiple genetically modified animals (e.g., a pig) can be born in a litter. A litter of genetically modified animal can have at least 30%, 50%, 60%, 80%, or 90% survival rate, e.g., number of animals in a litter that survive after birth divided by the total number of animals in the litter.

A genetically modified non-human animal can comprise reduced expression of one or more genes compared to a non-genetically modified counterpart animal. The reduction of expression of a gene can result from mutations on one or more alleles of the gene. For example, a genetically modified animal can comprise a mutation on two or more alleles of a gene. In some cases, such genetically modified animal can be a diploid animal.

A genetically modified non-human animal can comprise reduced expression of one or more genes compared to a non-genetically modified counterpart animal. A genetically modified non-human animal can comprise reduced expression of two or more genes compared to a non-genetically modified counterpart animal. A genetically modified animal can have a genomic disruption in at least one gene selected from a group consisting of a component of an MHC I-specific enhanceosome, a transporter of an MHC I-binding peptide, a natural killer (NK) group 2D ligand, a CXC chemokine receptor (CXCR)3 ligand, MHC II transactivator (CIITA), C3, an endogenous gene not expressed in a human, and any combination thereof.

In some cases a genetically modified animal has reduced expression of a gene in comparison to a non-genetically modified counterpart animal. In some cases, a genetically modified animal survives at least 22 days after birth. In other cases, a genetically modified animal can survive at least or at least about 23 to 30, 25 to 35, 35 to 45, 45 to 55, 55 to 65, 65 to 75, 75 to 85, 85 to 95, 95 to 105, 105 to 115, 115 to 225, 225 to 235, 235 to 245, 245 to 255, 255 to 265, 265 to 275, 275 to 285, 285 to 295, 295 to 305, 305 to 315, 315 to 325, 325 to 335, 335 to 345, 345 to 355, 355 to 365, 365 to 375, 375 to 385, 385 to 395, or 395 to 400 days after birth.

A non-genetically modified counterpart animal can be an animal substantially identical to the genetically modified animal but without genetic modification in the genome. For example, a non-genetically modified counterpart animal can be a wild-type animal of the same species as the genetically modified animal.

A genetically modified non-human animal can provide cells, tissues or organs for transplanting to a recipient or subject in need thereof. A recipient or subject in need thereof can be a recipient or subject known or suspected of having a condition. The condition can be treated, prevented, reduced, eliminated, or augmented by the methods and compositions disclosed herein. The recipient can exhibit low or no immuno-response to the transplanted cells, tissues or organs. The transplanted cells, tissues or organs can be non-recognizable by CD8+ T cells, NK cells, or CD4+ T cells of the recipient (e.g., a human or another animal). The genes whose expression is reduced can include MHC molecules, regulators of MHC molecule expression, and genes differentially expressed between the donor non-human animal and the recipient (e.g., a human or another animal). The reduced expression can be mRNA expression or protein expression of the one or more genes. For example, the reduced expression can be protein expression of the one or more genes. Reduced expression can also include no expression. For example an animal, cell, tissue or organ with reduced expression of a gene can have no expression (e.g., mRNA and/or protein expression) of the gene. Reduction of expression of a gene can inactivate the function of the gene. In some cases, when expression of a gene is reduced in a genetically modified animal, the expression of the gene is absent in the genetically modified animal.

A genetically modified non-human animal can comprise reduced expression of one or more MHC molecules compared to a non-genetically modified counterpart animal. For example, the non-human animal can be an ungulate, e.g., a pig, with reduced expression of one or more swine leukocyte antigen (SLA) class I and/or SLA class II molecules.

A genetically modified non-human animal can comprise reduced expression of any genes that regulate major histocompatibility complex (MHC) molecules (e.g., MHC I molecules and/or MHC II molecules) compared to a non-genetically modified counterpart animal. Reducing expression of such genes can result in reduced expression and/or function of MHC molecules (e.g., MHC I molecules and/or MHC II molecules). In some cases, the one or more genes whose expression is reduced in the non-human animal can comprise one or more of the following: components of an MHC I-specific enhanceosome, transporters of a MHC I-binding peptide, natural killer group 2D ligands, CXC chemical receptor (CXCR) 3 ligands, complement component 3 (C3), and major histocompatibility complex II transactivator (CIITA). In some cases, the component of a MHC I-specific enhanceosome can be NLRC5. In some cases, the component of a MHC I-specific enhanceosome can also comprise regulatory factor X (RFX) (e.g., RFX1), nuclear transcription factor Y (NFY), and cAMP response element-binding protein (CREB). In some instances, the transporter of a MHC I-binding peptide can be Transporter associated with antigen processing 1 (TAP1). In some cases, the natural killer (NK) group 2D ligands can comprise MICA and MICB. For example, the genetically modified non-human animal can comprise reduced expression of one or more of the following genes: NOD-like receptor family CARD domain containing 5 (NLRC5), Transporter associated with antigen processing 1 (TAP1), C—X—C motif chemokine 10 (CXCL10), MHC class I polypeptide-related sequence A (MICA), MHC class I polypeptide-related sequence B (MICB), complement component 3 (C3), and CIITA. A genetically modified animal can comprise reduced expression of one or more of the following genes: a component of an MHC I-specific enhanceosome (e.g., NLRC5), a transporter of an MHC I-binding peptide (TAP1), and C3.

A genetically modified non-human animal can comprise reduced expression compared to a non-genetically modified counterpart of one or more genes expressed at different levels between the non-human animal and a recipient receiving a cell, tissue, or organ from the non-human animal. For example, the one or more genes can be expressed at a lower level in a human than in the non-human animal. In some cases, the one or more genes can be endogenous genes of the non-human animal. The endogenous genes are in some cases genes not expressed in another species. For example, the endogenous genes of the non-human animal can be genes that are not expressed in a human. For example, in some cases, homologs (e.g., orthologs) of the one or more genes do not exist in a human. In another example, homologs (e.g., orthologs) of the one or more genes can exist in a human but are not expressed.

In some cases, a non-human animal can be a pig, and the recipient can be a human. In these cases, the one or more genes can be any genes expressed in a pig but not in a human. For example, the one or more genes can comprise glycoprotein galactosyltransferase alpha 1, 3 (GGTA1), putative cytidine monophosphate-N-acetylneuraminic acid hydroxylase-like protein (CMAH), and β1,4 N-acetylgalactosaminyltransferase (B4GALNT2). A genetically modified non-human animal can comprise reduced expression of B4GALNT2, GGTA1, or CMAH, where the reduced expression is in comparison to a non-genetically modified counterpart animal. A genetically modified non-human animal can comprise reduced expression of B4GALNT2 and GGTA1, where the reduced expression is in comparison to a non-genetically modified counterpart animal. A genetically modified non-human animal can comprise reduced expression of B4GALNT2 and CMAH, where the reduced expression is in comparison to a non-genetically modified counterpart animal. A genetically modified non-human animal can comprise reduced expression of B4GALNT2, GGTA1, and CMAH, where the reduced expression is in comparison to a non-genetically modified counterpart animal.

The genetically modified non-human animal can comprise reduced expression compared to a non-genetically modified counterpart of one or more of any of the genes disclosed herein, including NLRC5, TAP1, CXCL10, MICA, MICB, C3, CIITA, GGTA1, CMAH, and B4GALNT2.

A genetically modified non-human animal can comprise one or more genes whose expression is reduced, e.g., where genetic expression is reduced. The one or more genes whose expression is reduced include but are not limited to NOD-like receptor family CARD domain containing 5 (NLRC5), Transporter associated with antigen processing 1 (TAP1), Glycoprotein galactosyltransferase alpha 1,3 (GGTA1), Putative cytidine monophosphate-N-acetylneuraminic acid hydroxylase-like protein (CMAH), C—X—C motif chemokine 10 (CXCL10), MHC class I polypeptide-related sequence A (MICA), MHC class I polypeptide-related sequence B (MICB), class II major histocompatibility complex transactivator (CIITA), Beta-1,4-N-Acetyl-Galactosaminyl Transferase 2 (B4GALNT2), complemental component 3 (C3), and/or any combination thereof.

A genetically modified non-human animal can comprise 1, 2, 3, 4, 5, 6, 7, 8, 9, 10, 11, 12, 13, 14, 15, 16, 17, 18, 19, 20 or more genes whose expression is disrupted. For illustrative purposes, and not to limit various combinations a person of skill in the art can envision, a genetically modified non-human animal can have NLRC5 and TAP1 individually disrupted. A genetically modified non-human animal can also have both NLRC5 and TAP1 disrupted. A genetically modified non-human animal can also have NLRC5 and TAP1, and in addition to one or more of the following GGTA1, CMAH, CXCL10, MICA, MICB, B4GALNT2, or CIITA genes disrupted; for example “NLRC5, TAP1, and GGTA1” or “NLRC5, TAP1, and CMAH” can be disrupted. A genetically modified non-human animal can also have NLRC5, TAP1, GGTA1, and CMAH disrupted. Alternatively, a genetically modified non-human animal can also have NLRC5, TAP1, GGTA1, B4GALNT2, and CMAH disrupted. In some cases, a genetically modified non-human animal can have C3 and GGTA1 disrupted. In some cases, a genetically modified non-human animal can have reduced expression of NLRC5, C3, GGTA1, B4GALNT2, CMAH, and CXCL10. In some cases, a genetically modified non-human animal can have reduced expression of TAP1, C3, GGTA1, B4GALNT2, CMAH, and CXCL10. In some cases, a genetically modified non-human animal can have reduced expression of NLRC5, TAP1, C3, GGTA1, B4GALNT2, CMAH, and CXCL10. A B4GALNT2 gene can be a Gal2-2 or Gal 2-1.

Lack of MHC class I expression on transplanted human cells can cause the passive activation of natural killer (NK) cells (Ohlen et al., 1989). Lack of MHC class I expression could be due to NLRC5, TAP1, or B2M gene deletion. NK cell cytotoxicity can be overcome by the expression of the human MHC class 1 gene, HLA-E, can stimulate the inhibitory receptor CD94/NKG2A on NK cells to prevent cell killing (Weiss et al., 2009; Lilienfeld et al., 2007; Sasaki et al., 1999). Successful expression of the HLA-E gene can be dependent on co-expression of the human B2M (beta 2 microglobulin) gene and a cognate peptide (Weiss et al., 2009; Lilienfeld et al., 2007; Sasaki et al., 1999; Pascasova et al., 1999). A nuclease mediated break in the stem cell DNA can allow for the insertion of one or multiple genes via homology directed repair. The HLA-E and hB2M genes in series can be integrated in the region of the nuclease mediated DNA break thus preventing expression of the target gene (for example, NLRC5) while inserting the transgenes.

Expression levels of genes can be reduced to various extents. For example, expression of one or more genes can be reduced by or by about 100%. In some cases, expression of one or more genes can be reduced by or by about 99%, 95%, 90%, 85%, 80%, 75%, 70%, 65%, 60%, 55%, or 50% of normal expression, e.g., compared to the expression of non-modified controls. In some cases, expression of one or more genes can be reduced by at least or to at least about 99% to 90%; 89% to 80%, 79% to 70%; 69% to 60%; 59% to 50% of normal expression, e.g., compared to the expression of non-modified controls. For example, expression of one or more genes can be reduced by at least or at least about 90% or by at least or at least about 90% to 99% of normal expression.

Expression can be measured by any known method, such as quantitative PCR (qPCR), including but not limited to PCR, real-time PCR (e.g., Sybr-green), and/or hot PCR. In some cases, expression of one or more genes can be measured by detecting the level of transcripts of the genes. For example, expression of one or more genes can be measured by Northern blotting, nuclease protection assays (e.g., RNase protection assays), reverse transcription PCR, quantitative PCR (e.g., real-time PCR such as real-time quantitative reverse transcription PCR), in situ hybridization (e.g., fluorescent in situ hybridization (FISH)), dot-blot analysis, differential display, serial analysis of gene expression, subtractive hybridization, microarrays, nanostring, and/or sequencing (e.g., next-generation sequencing). In some cases, expression of one or more genes can be measured by detecting the level of proteins encoded by the genes. For example, expression of one or more genes can be measured by protein immunostaining, protein immunoprecipitation, electrophoresis (e.g., SDS-PAGE), Western blotting, bicinchoninic acid assay, spectrophotometry, mass spectrometry, enzyme assays (e.g., enzyme-linked immunosorbent assays), immunohistochemistry, flow cytometry, and/or immunoctyochemistry. Expression of one or more genes can also be measured by microscopy. The microscopy can be optical, electron, or scanning probe microscopy. Optical microscopy can comprise use of bright field, oblique illumination, cross-polarized light, dispersion staining, dark field, phase contrast, differential interference contrast, interference reflection microscopy, fluorescence (e.g., when particles, e.g., cells, are immunostained), confocal, single plane illumination microscopy, light sheet fluorescence microscopy, deconvolution, or serial time-encoded amplified microscopy. Expression of MHC I molecules can also be detected by any methods for testing expression as described herein.

Disrupted Genes

Cells, organs, and/or tissues having different combinations of disrupted genes described herein, can result in cells, organs, and/or tissues that are less susceptible to rejection when transplanted into a recipient. For example, the inventors have found that disrupting (e.g., reducing expression of) certain genes, such as NLRC5, TAP1, GGTA1, B4GALNT2, CMAH, CXCL10, MICA, MICB, C3, and/or CIITA, can increase the likelihood of graft survival. In some cases, at least two genes are disrupted. For example, GGTA1-10 and Gal2-2 can be disrupted. In some cases, GGTA1-10, Gal2-2, and NLRC5-6 can be disrupted. In other cases, NLRC5-6 and Gal2-2 can be disrupted.

In some cases, the disruptions are not limited to solely these genes. It is contemplated that genetic homologues (e.g., any mammalian version of the gene) of the genes within this applications are covered. For example, genes that are disrupted can exhibit a certain identity and/or homology to genes disclosed herein, e.g., NLRC5, TAP1, GGTA1, B4GALNT2, CMAH, CXCL10, MICA, MICB, C3, and/or CIITA. Therefore, it is contemplated that a gene that exhibits at least or at least about 50%, 55%, 60%, 65%, 70%, 75%, 80%, 85%, 90%, 95%, 99%, or 100% homology (at the nucleic acid or protein level) can be disrupted, e.g., a gene that exhibits at least or at least about from 50% to 60%; 60% to 70%; 70% to 80%; 80% to 90%; or 90% to 99% homology. It is also contemplated that a gene that exhibits at least or at least about 50%, 55%, 60%, 65%, 70%, 75%, 80%, 85%, 90%, 99%, or 100% identity (at the nucleic acid or protein level) can be disrupted, e.g., a gene that exhibits at least or at least about from 50% to 60%; 60% to 70%; 70% to 80%; 80% to 90%; or 90% to 99% identity. Some genetic homologues are known in the art, however, in some cases, homologues are unknown. However, homologous genes between mammals can be found by comparing nucleic acid (DNA or RNA) sequences or protein sequences using publically available databases such as NCBI BLAST. Genomic sequences, cDNA and protein sequences of exemplary genes are shown in Table 1.

Gene suppression can also be done in a number of ways. For example, gene expression can be reduced by knock out, altering a promoter of a gene, and/or by administering interfering RNAs (knockdown). This can be done at an organism level or at a tissue, organ, and/or cellular level. If one or more genes are knocked down in a non-human animal, cell, tissue, and/or organ, the one or more genes can be reduced by administrating RNA interfering reagents, e.g., siRNA, shRNA, or microRNA. For example, a nucleic acid which can express shRNA can be stably transfected into a cell to knockdown expression. Furthermore, a nucleic acid which can express shRNA can be inserted into the genome of a non-human animal, thus knocking down a gene with in a non-human animal.

Disruption methods can also comprise overexpressing a dominant negative protein. This method can result in overall decreased function of a functional wild-type gene. Additionally, expressing a dominant negative gene can result in a phenotype that is similar to that of a knockout and/or knockdown.

In some cases a stop codon can be inserted or created (e.g., by nucleotide replacement), in one or more genes, which can result in a nonfunctional transcript or protein (sometimes referred to as knockout). For example, if a stop codon is created within the middle of one or more genes, the resulting transcription and/or protein can be truncated, and can be nonfunctional. However, in some cases, truncation can lead to an active (a partially or overly active) protein. In some cases, if a protein is overly active, this can result in a dominant negative protein, e.g., a mutant polypeptide that disrupts the activity of the wild-type protein.

This dominant negative protein can be expressed in a nucleic acid within the control of any promoter. For example, a promoter can be a ubiquitous promoter. A promoter can also be an inducible promoter, tissue specific promoter, and/or developmental specific promoter.

The nucleic acid that codes for a dominant negative protein can then be inserted into a cell or non-human animal. Any known method can be used. For example, stable transfection can be used. Additionally, a nucleic acid that codes for a dominant negative protein can be inserted into a genome of a non-human animal.

One or more genes in a non-human animal can be knocked out using any method known in the art. For example, knocking out one or more genes can comprise deleting one or more genes from a genome of a non-human animal. Knocking out can also comprise removing all or a part of a gene sequence from a non-human animal. It is also contemplated that knocking out can comprise replacing all or a part of a gene in a genome of a non-human animal with one or more nucleotides. Knocking out one or more genes can also comprise inserting a sequence in one or more genes thereby disrupting expression of the one or more genes. For example, inserting a sequence can generate a stop codon in the middle of one or more genes. Inserting a sequence can also shift the open reading frame of one or more genes. In some cases, knock out can be performed in a first exon of a gene. In other cases, knock out can be performed in a second exon of a gene.

Knockout can be done in any cell, organ, and/or tissue in a non-human animal. For example, knockout can be whole body knockout, e.g., expression of one or more genes is reduced in all cells of a non-human animal. Knockout can also be specific to one or more cells, tissues, and/or organs of a non-human animal. This can be achieved by conditional knockout, where expression of one or more genes is selectively reduced in one or more organs, tissues or types of cells. Conditional knockout can be performed by a Cre-lox system, where cre is expressed under the control of a cell, tissue, and/or organ specific promoter. For example, one or more genes can be knocked out (or expression can be reduced) in one or more tissues, or organs, where the one or more tissues or organs can include brain, lung, liver, heart, spleen, pancreas, small intestine, large intestine, skeletal muscle, smooth muscle, skin, bones, adipose tissues, hairs, thyroid, trachea, gall bladder, kidney, ureter, bladder, aorta, vein, esophagus, diaphragm, stomach, rectum, adrenal glands, bronchi, ears, eyes, retina, genitals, hypothalamus, larynx, nose, tongue, spinal cord, or ureters, uterus, ovary, testis, and/or any combination thereof. One or more genes can also be knocked out (or expression can be reduced) in one types of cells, where one or more types of cells include trichocytes, keratinocytes, gonadotropes, corticotropes, thyrotropes, somatotropes, lactotrophs, chromaffin cells, parafollicular cells, glomus cells melanocytes, nevus cells, merkel cells, odontoblasts, cementoblasts corneal keratocytes, retina muller cells, retinal pigment epithelium cells, neurons, glias (e.g., oligodendrocyte astrocytes), ependymocytes, pinealocytes, pneumocytes (e.g., type I pneumocytes, and type II pneumocytes), clara cells, goblet cells, G cells, D cells, Enterochromaffin-like cells, gastric chief cells, parietal cells, foveolar cells, K cells, D cells, I cells, goblet cells, paneth cells, enterocytes, microfold cells, hepatocytes, hepatic stellate cells (e.g., Kupffer cells from mesoderm), cholecystocytes, centroacinar cells, pancreatic stellate cells, pancreatic α cells, pancreatic β cells, pancreatic δ cells, pancreatic F cells, pancreatic ε cells, thyroid (e.g., follicular cells), parathyroid (e.g., parathyroid chief cells), oxyphil cells, urothelial cells, osteoblasts, osteocytes, chondroblasts, chondrocytes, fibroblasts, fibrocytes, myoblasts, myocytes, myosatellite cells, tendon cells, cardiac muscle cells, lipoblasts, adipocytes, interstitial cells of cajal, angioblasts, endothelial cells, mesangial cells (e.g., intraglomerular mesangial cells and extraglomerular mesangial cells), juxtaglomerular cells, macula densa cells, stromal cells, interstitial cells, telocytes simple epithelial cells, podocytes, kidney proximal tubule brush border cells, sertoli cells, leydig cells, granulosa cells, peg cells, germ cells, spermatozoon ovums, lymphocytes, myeloid cells, endothelial progenitor cells, endothelial stem cells, angioblasts, mesoangioblasts, pericyte mural cells, and/or any combination thereof.

Conditional knockouts can be inducible, for example, by using tetracycline inducible promoters, development specific promoters. This can allow for eliminating or suppressing expression of a gene/protein at any time or at a specific time. For example, with the case of a tetracycline inducible promoter, tetracycline can be given to a non-human animal any time after birth. If a non-human animal is a being that develops in a womb, then promoter can be induced by giving tetracycline to the mother during pregnancy. If a non-human animal develops in an egg, a promoter can be induced by injecting, or incubating in tetracycline. Once tetracycline is given to a non-human animal, the tetracycline will result in expression of cre, which will then result in excision of a gene of interest.

A cre/lox system can also be under the control of a developmental specific promoter. For example, some promoters are turned on after birth, or even after the onset of puberty. These promoters can be used to control cre expression, and therefore can be used in developmental specific knockouts.

It is also contemplated that any combinations of knockout technology can be combined. For example, tissue specific knockout can be combined with inducible technology, creating a tissue specific, inducible knockout. Furthermore, other systems such developmental specific promoter, can be used in combination with tissues specific promoters, and/or inducible knockouts.

In some cases, gene editing can be useful to design a knockout. For example, gene editing can be performed using a nuclease, including CRISPR associated proteins (Cas proteins, e.g., Cas9), Zinc finger nuclease (ZFN), Transcription Activator-Like Effector Nuclease (TALEN), and maganucleases. Nucleases can be naturally existing nucleases, genetically modified, and/or recombinant. For example, a CRISPR/Cas system can be suitable as a gene editing system.

It is also contemplated that less than all alleles of one or more genes of a non-human animal can be knocked out. For example, in diploid non-human animals, it is contemplated that one of two alleles are knocked out. This can result in decreased expression and decreased protein levels of genes. Overall decreased expression can be less than or less than about 99%, 95%, 90%, 85%, 80%, 75%, 70%, 65%, 60%, 55%, 50%, 45%, 40%, 35%, 30%, 25%, or 20%; e.g., from or from about 99% to 90%; 90% to 80%; 80% to 70%; 70% to 60%; 60% to 50%; 50% to 40%; 40% to 30%, or 30% to 20%; compared to when both alleles are functioning, for example, not knocked out and/or knocked down. Additionally, overall decrease in protein level can be the same as the decreased in overall expression. Overall decrease in protein level can be about or less than about 99%, 95%, 90%, 80%, 70%, 60%, 50%, 40%, 30%, or 20%, e.g., from or from about 99% to 90%; 90% to 80%; 80% to 70%; 70% to 60%; 60% to 50%; 50% to 40%; 40% to 30%, or 30% to 20%; compared to when both alleles are functioning, for example, not knocked out and/or knocked down. However, it is also contemplated that all alleles of one or more genes in a non-human animal can be knocked out.

Knockouts of one or more genes can be verified by genotyping. Methods for genotyping can include sequencing, restriction fragment length polymorphism identification (RFLPI), random amplified polymorphic detection (RAPD), amplified fragment length polymorphism detection (AFLPD), PCR (e.g., long range PCR, or stepwise PCR), allele specific oligonucleotide (ASO) probes, and hybridization to DNA microarrays or beads. For example, genotyping can be performed by sequencing. In some cases, sequencing can be high fidelity sequencing. Methods of sequencing can include Maxam-Gilbert sequencing, chain-termination methods (e.g., Sanger sequencing), shotgun sequencing, and bridge PCR. In some cases, genotyping can be performed by next-generation sequencing. Methods of next-generation sequencing can include massively parallel signature sequencing, colony sequencing, pyrosequencing (e.g., pyrosequencing developed by 454 Life Sciences), single-molecule rea-time sequencing (e.g., by Pacific Biosciences), Ion semiconductor sequencing (e.g., by Ion Torrent semiconductor sequencing), sequencing by synthesis (e.g., by Solexa sequencing by Illumina), sequencing by ligation (e.g., SOLiD sequencing by Applied Biosystems), DNA nanoball sequencing, and heliscope single molecule sequencing. In some cases, genotyping of a non-human animal herein can comprise full genome sequencing analysis. In some cases, knocking out of a gene in an animal can be validated by sequencing (e.g., next-generation sequencing) a part of the gene or the entire gene. For example, knocking out of NLRC5 gene in a pig can be validated by next generation sequencing of the entire NLRC5. The next generation sequencing of NLRC5 can be performed using e.g. using forward primer 5′-gctgtggcatatggcagttc-3′ (SEQ ID No. 1) and reverse primer 5′-tccatgtataagtctttta-3′ (SEQ ID No. 2), or forward primer 5′-ggcaatgccagatcctcaac-3′ (SEQ ID No. 3) and reverse primer 5′-tgtctgatgtctttctcatg-3′ (SEQ ID No. 4).

TABLE 1
Genomic sequences, cDNA and proteins of exemplary disrupted genes*

| | Genomic | | | | |
|---|---|---|---|---|---|
| | sequence | cDNA | | protein | |
| | SEQ ID | SEQ ID | | SEQ ID | |
| Gene | No. | No. | Accession No. | No. | Accession No. |
| NLRC5 | 5 | 6 | KC514136.1 | 7 | AGG68119.1 |
| TAP1 | 8 | 9 | NM_001044581 | 10 | NP_001038046.1 |
| GGTA1 | 11 | 12 | AF221508 | 13 | NP_998975.1 |
| CMAH | 14 | 15 | NM_001113015 | 16 | NP_001106486.1 |
| CXCL10 | 17 | 18 | NM_001008691.1 | 19 | NP_001008691.1 |
| CIITA | 20 | 21 | XM_013995652.1 | 22 | XP_013851106.1 |
| B4GALNT2 | 23 | 24 | NM_001244330.1 | 25 | NP_001231259.1 |
| C3 | 26 | 27 | NM_214009.1 | 28 | NP_999174.1 |
| MICA | 29 | 30 | NM_000247.2 | 31 | NP_000238.1 |
| MICB | 32 | 33 | NM_001289160.1 | 34 | NP_001276089.1 |

*The sequences for Table 1 can be found in Table 18.

Transgenes

Transgenes, or exogenous nucleic acid sequences, can be useful for overexpressing endogenous genes at higher levels than without the transgenes. Additionally, exogenous nucleic acid sequences can be used to express exogenous genes. Transgenes can also encompass other types of genes, for example, a dominant negative gene.

A transgene of protein X can refer to a transgene comprising an exogenous nucleic acid sequence encoding protein X. As used herein, in some cases, a transgene encoding protein X can be a transgene encoding 100% or about 100% of the amino acid sequence of protein X. In some cases, a transgene encoding protein X can encode the full or partial amino sequence of protein X. For example, the transgene can encode at least or at least about 99%, 95%, 90%, 80%, 70%, 60%, 50%, 40%, 30%, 20%, 10%, or 5%, e.g., from or from about 99% to 90%; 90% to 80%; 80% to 70%; 70% to 60%; or 60% to 50%; of the amino acid sequence of protein X. Expression of a transgene can ultimately result in a functional protein, e.g., a partially or fully functional protein. As discussed above, if a partial sequence is expressed, the ultimate result can be in some cases a nonfunctional protein or a dominant negative protein. A nonfunctional protein or dominant negative protein can also compete with a functional (endogenous or exogenous) protein. A transgene can also encode an RNA (e.g., mRNA, shRNA, siRNA, or microRNA). In some cases, where a transgene encodes for an mRNA, this can in turn be translated into a polypeptide (e.g., a protein). Therefore, it is contemplated that a transgene can encode for protein. A transgene can, in some instances, encode a protein or a portion of a protein. Additionally, a protein can have one or more mutations (e.g., deletion, insertion, amino acid replacement, or rearrangement) compared to a wild-type polypeptide. A protein can be a natural polypeptide or an artificial polypeptide (e.g., a recombinant polypeptide). A transgene can encode a fusion protein formed by two or more polypeptides.

Where a transgene, or exogenous nucleic acid sequence, encodes for an mRNA based on a naturally occurring mRNA (e.g., an mRNA normally found in another species), the mRNA can comprise one or more modifications in the 5′ or 3′ untranslated regions. The one or more modifications can comprise one or more insertions, on or more deletions, or one or more nucleotide changes, or a combination thereof. The one or more modifications can increase the stability of the mRNA. The one or more modifications can remove a binding site for an miRNA molecule, such as an miRNA molecule that can inhibit translation or stimulate mRNA degradation. For example, an mRNA encoding for a HLA-G protein can be modified to remove a biding site for an miR148 family miRNA. Removal of this binding site can increase mRNA stability.

Transgenes can be placed into an organism, cell, tissue, or organ, in a manner which produces a product of the transgene. For example, disclosed herein is a non-human animal comprising one or more transgenes. One or more transgenes can be in combination with one or more disruptions as described herein. A transgene can be incorporated into a cell. For example, a transgene can be incorporated into an organism's germ line. When inserted into a cell, a transgene can be either a complementary DNA (cDNA) segment, which is a copy of messenger RNA (mRNA), or a gene itself residing in its original region of genomic DNA (with or without introns).

A transgene can comprise a polynucleotide encoding a protein of a species and expressing the protein in an animal of a different species. For example, a transgene can comprise a polynucleotide encoding a human protein. Such a polynucleotide can be used express the human protein (e.g., CD47) in a non-human animal (e.g., a pig). In some cases, the polynucleotide can be synthetic, e.g., different from any native polynucleotide in sequence and/or chemical characteristics.

The polynucleotide encoding a protein of species X can be optimized to express the protein in an animal of a species Y. There may be codon usage bias (e.g., differences in the frequency of occurrence of synonymous codons in coding DNA). A codon can be a series of nucleotides (e.g., a series of 3 nucleotides) that encodes a specific amino acid residue in a polypeptide chain or for the termination of translation (stop codons). Different species may have different preference in the DNA codons. The optimized polynucleotide can encode a protein of species X, in some cases with codons of a species Y, so that the polynucleotide can express the protein more efficiently in the species Y, compared to the native gene encoding the protein of species X. In some cases, an optimized polynucleotide can express a protein at least 5%, 10%, 20%, 40%, 80%, 90%, 1.5 folds, 2 folds, 5 folds, or 10 folds more efficiently in species Y than a native gene of species X encoding the same protein.

Human Leukocyte Antigen G (HLA-G)

HLA-G can be a potent immuno-inhibitory and tolerogenic molecule. HLA-G expression in a human fetus can enable the human fetus to elude the maternal immune response. Neither stimulatory functions nor responses to allogeneic HLA-G have been reported to date. HLA-G can be a non-classical HLA class I molecule. It can differ from classical MHC class I molecules by its genetic diversity, expression, structure, and function. HLA-G can be characterized by a low allelic polymorphism. Expression of HLA-G can be restricted to trophoblast cells, adult thymic medulla, and stem cells. However, HLA-G neo-expression may be induced in pathological conditions such as cancers, multiple sclerosis, inflammatory diseases, or viral infections.

Seven isoforms of HLA-G have been identified. The different isoforms can be products of alternative splicing. Four of these can be membrane bound (HLA-G1 to -G4), and 3 can be soluble isoforms (HLA-G5 to -G7). HLA-G1 and HLA-G5 isoforms present the typical structure of the classical HLA class I molecules formed by a 3 globular domain (α1-α3) heavy-chain, noncovalently associated to β-2-microglobulin (B2M) and a nonapeptide. The truncated isoforms lack 1 or 2 domains, although they all contain the al domain, and they are all B2M-free isoforms.

HLA-G can exerts an immuno-inhibitory function through direct binding to inhibitory receptors, e.g., ILT2/CD85j/LILRB1, ILT4/CD85d/LILRB2, or KIR2DL4/CD158d.

ILT2 can be expressed by B cells, some T cells, some NK cells, and monocytes/dendritic cells. ILT4 can be myeloid-specific and its expression can be restricted to monocytes/dendritic cells. KIR2DL4 can be a specific receptor for HLA-G. It can be expressed by the CD56^{bright} subset of NK cells. ILT2 and ILT4 receptors can bind a wide range of classical HLA molecules through the α3 domain and B2M. However, HLA-G can be their ligand of highest affinity.

ILT2-HLA-G interaction can mediate the inhibition of, for example: i) NK and antigen-specific CD8+ T cell cytolytic function, ii) alloproliferative response of CD4+T cells, and iii) maturation and function of dendritic cells. ILT2-HLA-G interaction can impede both naïve and memory B cell function in vitro and in vivo. HLA-G can inhibit B cell proliferation, differentiation, and Ig secretion in both T cell-dependent and -independent models of B cell activation. HLA-G can act as a negative B cell regulator in modulating B cell Ab secretion. HLA-G can also induce the differentiation of regulatory T cells, which can then inhibit allogeneic responses themselves may participate in the tolerance of allografts.

The expression of HLA-G by tumor cells can enable the escape of immunosurveillance mediated by host T lymphocytes and NK cells. Thus, the expression of HLA-G by malignant cells may prevent tumor immune eradication by inhibiting the activity of tumor-infiltrating NK cells, cytotoxic T lymphocytes (CTLs), and antigen presenting cells (APCs).

The HLA-G structure variation, particularly its monomeric/multimeric status and its association with B2M, can play a role in the biological function of HLA-G, its regulation and its interactions with the inhibitory receptors ILT2 and ILT4.

ILT2 and ILT4 inhibitory receptors may have a higher affinity for HLA-G multimers than monomeric structures. HLA-G1 and HLA-G5 (HLA-G1/5) can form dimers through disulphide bonds between unique cysteine residues at positions 42 (Cys42-Cys42), within the al domain. Dimers of B2M-associated HLA-G1 may bind ILT2 and ILT4 with higher affinity than monomers. This increased affinity of dimers may be due to an oblique orientation that exposes the ILT2- and ILT4-binding sites of the α3 domain, making it more accessible to the receptors. Both ILT2 and ILT4 can bind the HLA-G α3 domain at the level of F195 and Y197 residues.

ILT2 and ILT4 bind differently to their HLA-G isoforms. ILT2 may recognize only B2M-associated HLA-G structures, whereas ILT4 may recognize both B2M-associated and B2M-free HLA-G heavy chains. B2M-free heavy chains have been detected at the cell surface and in culture supernatants of HLA-G-expressing cells. Furthermore, B2M-free HLA-G heavy chains may be the main structure produced by human villous trophoblast cells. The presence of (B2M-free) α1-α3 structures (HLA-G2 and G-6 isoforms) was shown in the circulation of human heart transplant recipients and may be associated with better allograft acceptance. The α1-α3 structure may bind only to ILT4 but not ILT2. However, α1-α3 dimers (with dimerization of α1-α3 monomers achieved through disulfide bonds between two free cysteines in position 42) may be tolerogenic in vivo in an allogeneic murine skin transplantation model. An (α1-α3)×2 synthetic molecule may inhibit the proliferation of tumor cell lines that did not express ILT4. This may indicate the existence of yet unknown receptors for HLA-G.

Accordingly, in one aspect, disclosed herein are genetically modified non-human animals and cells comprising an exogenous nucleic acid sequence encoding for an HLA-G protein. The genetically modified non-human animals and cells can also comprise one or more additional genetic modifications, such as any of the genetic modifications (e.g., knock-ins, knock-outs, gene disruptions, etc.) disclosed herein. For example, the genetically modified non-human animals and cells can also comprise another exogenous nucleic acid sequence encoding a B2M protein.

A non-human animal can comprise one or more transgenes comprising one or more polynucleotide inserts. The polynucleotide inserts can encode one or proteins or functional fragments thereof. For example, a non-human genetically modified animal can comprise one or more exogenous nucleic acid sequences encoding one or more proteins or functional fragments thereof. In some cases, a non-human animal can comprise one or more transgenes comprising one or more polynucleotide inserts encoding proteins that can reduce expression and/or function of MHC molecules (e.g., MHC I molecules and/or MHC II molecules). The one or more transgenes can comprise one or more polynucleotide inserts encoding MHC I formation suppressors, regulators of complement activations, inhibitory ligands for NK cells, B7 family members, CD47, serine protease inhibitors, galectins, and/or any fragments thereof. In some cases, the MHC I formation suppressors can be infected cell protein 47 (ICP47). In some cases, regulators of complement activation can comprise cluster of differentiation 46 (CD46), cluster of differentiation 55 (CD55), and cluster of differentiation 59 (CD59). In some cases, inhibitory ligands for NK cells can comprise leukocyte antigen E (HLA-E), human leukocyte antigen G (HLA-G), and β-2-microglobulin (B2M). An inhibitory ligand for NK cells can be an isoform of HLA-G, e.g., HLA-G1, HLA-G2, HLA-G3, HLA-G4, HLA-G5, HLA-G6, or HLA-G7. For example, inhibitory ligand for NK cells can be HLA-G1. A transgene of HLA-G (e.g., HLA-G1, HLA-G2, HLA-G3, HLA-G4, HLA-G5, HLA-G6, or HLA-G7) can refer to a transgene comprising a nucleotide sequence encoding HLA-G (e.g., HLA-G1, HLA-G2, HLA-G3, HLA-G4, HLA-G5, HLA-G6, or HLA-G7). As used herein, in some cases, a transgene encoding HLA-G (e.g., HLA-G1, HLA-G2, HLA-G3, HLA-G4, HLA-G5, HLA-G6, or HLA-G7) can be a transgene encoding 100% or about 100% of the amino acid sequence of HLA-G (e.g., HLA-G1, HLA-G2, HLA-G3, HLA-G4, HLA-G5, HLA-G6, or HLA-G7). In other cases, a transgene encoding HLA-G (e.g., HLA-G1, HLA-G2, HLA-G3, HLA-G4, HLA-G5, HLA-G6, or HLA-G7) can be a transgene encoding the full or partial sequence of HLA-G (e.g., HLA-G1, HLA-G2, HLA-G3, HLA-G4, HLA-G5, HLA-G6, or HLA-G7). For example, the transgene can encode at least or at least about 99%, 95%, 90%, 80%, 70%, 60%, or 50% of the amino acid sequence of HLA-G (e.g., HLA-G1, HLA-G2, HLA-G3, HLA-G4, HLA-G5, HLA-G6, or HLA-G7). For example, the transgene can encode 90% of the HLA-G amino acid sequence. A transgene can comprise polynucleotides encoding a functional (e.g., a partially or fully functional) HLA-G (e.g., HLA-G1, HLA-G2, HLA-G3, HLA-G4, HLA-G5, HLA-G6, or HLA-G7). In some cases, the one or more transgenes can comprise one or more polynucleotide inserts encoding one or more of ICP47, CD46, CD55, CD59, HLA-E, HLA-G (e.g., HLA-G1, HLA-G2, HLA-G3, HLA-G4, HLA-G5, HLA-G6, or HLA-G7), and B2M. The HLA-G genomic DNA sequence can have 8 exons by which alternative splicing results in 7 isoforms. The HLA-G1 isoform can exclude exon 7. The HLA-G2 isoform can exclude exon 3 and 7. Translation of intron 2 or intron 4 can result secreted isoforms due to the loss of the transmembrane domain expression. The maps of the genomic sequence and cDNA of HLA-G are shown in FIGS. 14A-14B. In some cases, B7 family members can comprise CD80, CD86, programmed death-ligand 1 (PD-L1), programmed death-ligand 2 (PD-L2), CD275, CD276, V-set domain containing T cell activation inhibitor 1 (VTCN1), platelet receptor Gi24, natural cytotoxicity triggering receptor 3 ligand 1 (NR3L1), and HERV-H LTR-associating 2 (HHLA2). For example, a B7 family member can be PD-L1 or PD-L2. In some cases, a serine protease inhibitor can be serine protease inhibitor 9 (Spi9). In some cases, galectins can comprise galectin-1, galectin-2, galectin-3, galectin-4, galectin-5, galectin-6, galectin-7, galectin-8, galectin-9, galectin-10, galectin-11, galectin-12, galectin-13, galectin-14, and galectin-15. For example, a galectin can be galectin-9.

A genetically modified non-human animal can comprise reduced expression of one or more genes and one or more transgenes disclosed herein. In some cases, a genetically modified non-human animal can comprise reduced expression of one or more of NLRC5, TAP1, CXCL10, MICA, MICB, C3, CIITA, GGTA1, CMAH, and B4GALNT2, and one or more transgenes comprising one or more polynucleotide inserts encoding one or more of ICP47, CD46, CD55, CD59, HLA-E, HLA-G (e.g., HLA-G1, HLA-G2, HLA-G3, HLA-G4, HLA-G5, HLA-G6, or HLA-G7), B2M, PD-L1, PD-L2, CD47, Spi9, and galectin-9. In some cases, a genetically modified non-human animal can comprise reduced expression GGTA1, CMAH, and B4GALNT2, and exogenous polynucleotides encoding HLA-G (e.g., HLA-G1, HLA-G2, HLA-G3, HLA-G4, HLA-G5, HLA-G6, or HLA-G7), CD47 (e.g., human CD47), PD-L1 (e.g., human PD-L1), and PD-L2 (e.g., human PD-L2). In some cases, a genetically modified non-human animal can comprise reduced expression GGTA1, CMAH, and B4GALNT2, and exogenous polynucleotides encoding HLA-E, CD47 (e.g., human CD47), PD-L1 (e.g., human PD-L1), and PD-L2 (e.g., human PD-L2). In some cases, a genetically modified non-human animal can comprise reduced expression NLRC5, C3, CXC10, GGTA1, CMAH, and B4GALNT2, and exogenous polynucleotides encoding HLA-G (e.g., HLA-G1, HLA-G2, HLA-G3, HLA-G4, HLA-G5, HLA-G6, or HLA-G7), CD47 (e.g., human CD47), PD-L1 (e.g., human PD-L1), and PD-L2 (e.g., human PD-L2). In some cases, a genetically modified non-human animal can comprise reduced expression TAP1, C3, CXC10GGTA1, CMAH, and B4GALNT2, and exogenous polynucleotides encoding HLA-G (e.g., HLA-G1, HLA-G2, HLA-G3, HLA-G4, HLA-G5, HLA-G6, or HLA-G7), CD47 (e.g., human CD47), PD-L1 (e.g., human PD-L1), and PD-L2 (e.g., human PD-L2). In some cases, a genetically modified non-human animal can comprise reduced expression NLRC5, C3, CXC10, GGTA1, CMAH, and B4GALNT2, and exogenous polynucleotides encoding HLA-E, CD47 (e.g., human CD47), PD-L1 (e.g., human PD-L1), and PD-L2 (e.g., human PD-L2). In some cases, a genetically modified non-human animal can comprise reduced expression TAP1, C3, CXC10, GGTA1, CMAH, and B4GALNT2, and exogenous polynucleotides encoding HLA-E. In some cases, a genetically modified non-human animal can comprise reduced expression of GGTA1 and a transgene comprising one or more polynucleotide inserts encoding HLA-E. In some cases, a genetically modified non-human animal can comprise reduced expression of GGTA1 and a transgene comprising one or more polynucleotide inserts encoding HLA-G (e.g., HLA-G1, HLA-G2, HLA-G3, HLA-G4, HLA-G5, HLA-G6, or HLA-G7). In some cases, a genetically modified non-human animal can comprise a transgene comprising one or more polynucleotide inserts encoding HLA-G (e.g., HLA-G1, HLA-G2, HLA-G3, HLA-G4, HLA-G5, HLA-G6, or HLA-G7) inserted adjacent to a Rosa26 promoter, e.g., a porcine Rosa26 promoter. In some cases, a genetically modified non-human animal can comprise reduced expression of NLRC5, C3, GGTA1, CMAH, and B4GALNT2, and transgenes comprising polynucleotides encoding proteins or functional fragments thereof, where the proteins comprise HLA-G1, Spi9, PD-L1, PD-L2, CD47, and galectin-9. In some cases, a genetically modified non-human animal can comprise reduced expression of TAP1, C3, GGTA1, CMAH, and B4GALNT2, and transgenes comprising polynucleotides encoding proteins or functional fragments thereof, where the proteins comprise HLA-G1, Spi9, PD-L1, PD-L2, CD47, and galectin-9. In some cases, a genetically modified non-human animal can comprise reduced expression of NLRC5, TAP1, C3, GGTA1, CMAH, and B4GALNT2, and transgenes comprising polynucleotides encoding proteins or functional fragments thereof, where the proteins comprise HLA-G1, Spi9, PD-L1, PD-L2, CD47, and galectin-9. In some cases, a genetically modified non-human animal can comprise reduced protein expression of NLRC5, C3, GGTA1, and CXCL10, and transgenes comprising polynucleotides encoding proteins or functional fragments thereof, where the protein comprise HLA-G1 or HLA-E. In some cases, a genetically modified non-human animal can comprise reduced protein expression of TAP1, C3, GGTA1, and CXCL10, and transgenes comprising polynucleotides encoding proteins or functional fragments thereof, where the protein comprise HLA-G1 or HLA-E. In some cases, a genetically modified non-human animal can comprise reduced protein expression of NLRC5, TAP1, C3, GGTA1, and CXCL10, and transgenes comprising polynucleotides encoding proteins or functional fragments thereof, where the protein comprise HLA-G1 or HLA-E. In some cases, CD47, PD-L1, and PD-L2 encoded by the transgenes herein can be human CD47, human PD-L1 and human PD-L2.

A genetically modified non-human animal can comprise a transgene inserted in a locus in the genome of the animal. In some cases, a transgene can be inserted adjacent to the promoter of or inside a targeted gene. In some cases, insertion of the transgene can reduce the expression of the targeted gene. The targeted gene can be a gene whose expression is reduced disclosed herein. For example, a transgene can be inserted adjacent to the promoter of or inside one or more of NLRC5, TAP1, CXCL10, MICA, MICB, C3, CIITA, GGTA1, CMAH, and B4GALNT2. In some cases, a transgene can be inserted adjacent to the promoter of or inside GGTA1. In some cases, a transgene (e.g., a CD47 transgene) can be inserted adjacent to a promoter that allows the transgene to selectively expression in certain types of cells. For example, a CD47 transgene can be inserted adjacent to promoter that allows the CD47 transgene to selectively express in blood cells and splenocytes. One of such promoters can be GGTA1 promoters.

For example, a non-human animal can comprise one or more transgenes (e.g., exogenous nucleic acid sequences) comprising one or more polynucleotide inserts of Infected cell protein 47 (ICP47), Cluster of differentiation 46 (CD46), Cluster of differentiation 55 (CD55), Cluster of differentiation 59 (CD 59), HLA-E, HLA-G (e.g., HLA-G1, HLA-G2, HLA-G3, HLA-G4, HLA-G5, HLA-G6, or HLA-G7), B2M, Spi9, PD-L1, PD-L2, CD47, galectin-9, any functional fragments thereof, or any combination thereof. Polynucleotide encoding for ICP47, CD46, CD55, CD59, HLA-E, HLA-G (e.g., HLA-G1, HLA-G2, HLA-G3, HLA-G4, HLA-G5, HLA-G6, or HLA-G7), or B2M can encode one or more of ICP47, CD46, CD55, CD59, HLA-E, HLA-G (e.g., HLA-G1, HLA-G2, HLA-G3, HLA-G4, HLA-G5, HLA-G6, or HLA-G7), B2M, Spi9, PD-L1, PD-L2, CD47, or galectin-9 human proteins. A non-human animal can comprise 1, 2, 3, 4, 5, 6, 7, 8, 9, 10, 11, 12, 13, 14, 15, 16, 17, 18, 19, 20, or more transgenes. For example, a non-human animal can comprise one or more transgene comprising ICP47, CD46, CD55, CD59, HLA-E, HLA-G (e.g., HLA-G1, HLA-G2, HLA-G3, HLA-G4, HLA-G5, HLA-G6, or HLA-G7), B2M, Spi9, PD-L1, PD-L2, CD47, galectin-9, any functional fragments thereof, or any combination thereof. A non-human animal can also comprise a single transgene encoding ICP47. A non-human animal can sometimes comprise a single transgene encoding CD59. A non-human animal can sometimes comprise a single transgene encoding HLA-G (e.g., HLA-G1, HLA-G2, HLA-G3, HLA-G4, HLA-G5, HLA-G6, or HLA-G7). A non-human animal can sometimes comprise a single transgene encoding HLA-E. A non-human animal can sometimes comprise a single transgene encoding B2M. A non-human animal can also comprise two or more transgenes, where the two or more transgenes are ICP47, CD46, CD55, CD59, and/or any combination thereof. For example, two or more transgenes can comprise CD59 and CD46 or CD59 and CD55. A non-human animal can also comprise three or more transgenes, where the three or more transgenes can comprise ICP47, CD46, CD55, CD59, or any combination thereof. For example, three or more transgenes can comprise CD59, CD46, and CD55. A non-human animal can also comprise four or more transgenes, where the four or more transgenes can comprise ICP47, CD46, CD55, and CD59. A non-human animal can comprise four or more transgenes comprising ICP47, CD46, CD55, and CD59.

A combination of transgenes and gene disruptions can be used. A non-human animal can comprise one or more reduced genes and one or more transgenes. For example, one or more genes whose expression is reduced can comprise any one of NLRC5, TAP1, GGTA1, B4GALNT2, CMAH, CXCL10, MICA, MICB, C3, CIITA, and/or any combination thereof, and one or more transgene can comprise ICP47, CD46, CD55, CD 59, any functional fragments thereof, and/or any combination thereof. For example, solely to illustrate various combinations, one or more genes whose expression is disrupted can comprise NLRC5 and one or more transgenes comprise ICP47. One or more genes whose expression is disrupted can also comprise TAP1, and one or more transgenes comprise ICP47. One or more genes whose expression is disrupted can also comprise NLRC5 and TAP1, and one or more transgenes comprise ICP47. One or more genes whose expression is disrupted can also comprise NLRC5, TAP1, and GGTA1, and one or more transgenes comprise ICP47. One or more genes whose expression is disrupted can also comprise NLRC5, TAP1, B4GALNT2, and CMAH, and one or more transgenes comprise ICP47. One or more genes whose expression is disrupted can also comprise NLRC5, TAP1, GGTA1, B4GALNT2, and CMAH, and one or more transgenes comprise ICP47. One or more genes whose expression is disrupted can also comprise NLRC5 and one or more transgenes comprise CD59. One or more genes whose expression is disrupted can also comprise TAP1, and one or more transgenes comprise CD59. One or more genes whose expression is disrupted can also comprise NLRC5 and TAP1, and one or more transgenes comprise CD59. One or more genes whose expression is disrupted can also comprise NLRC5, TAP1, and GGTA1, and one or more transgenes comprise CD59. One or more genes whose expression is disrupted can also comprise NLRC5, TAP1, B4GALNT2, and CMAH, and one or more transgenes comprise CD59. One or more genes whose expression is disrupted can also comprise NLRC5, TAP1, GGTA1, B4GALNT2, and CMAH, and one or more transgenes comprise CD59.

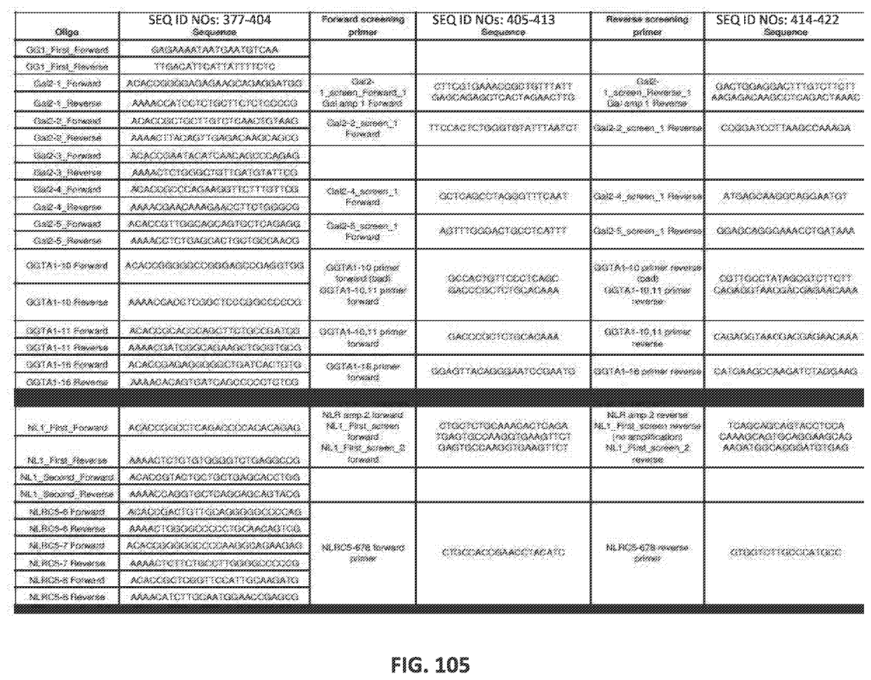

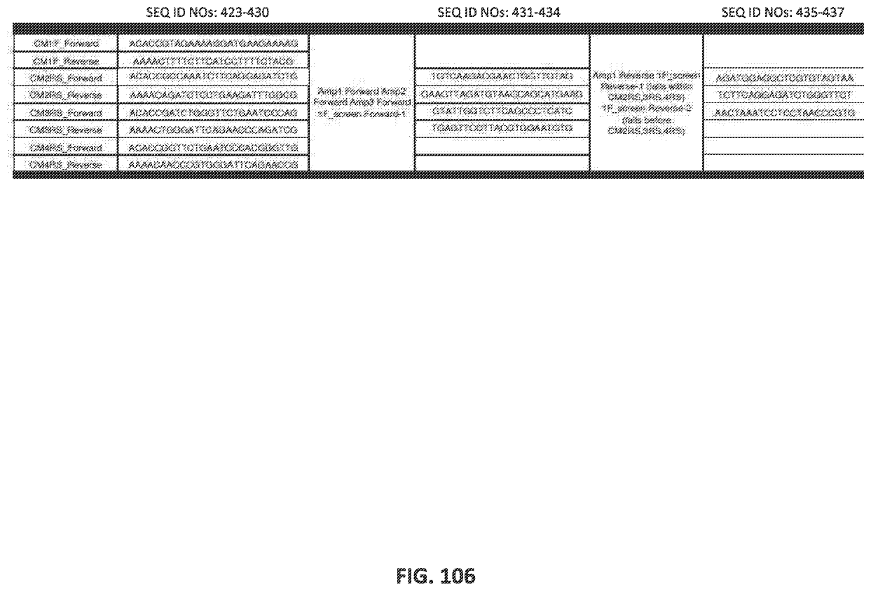

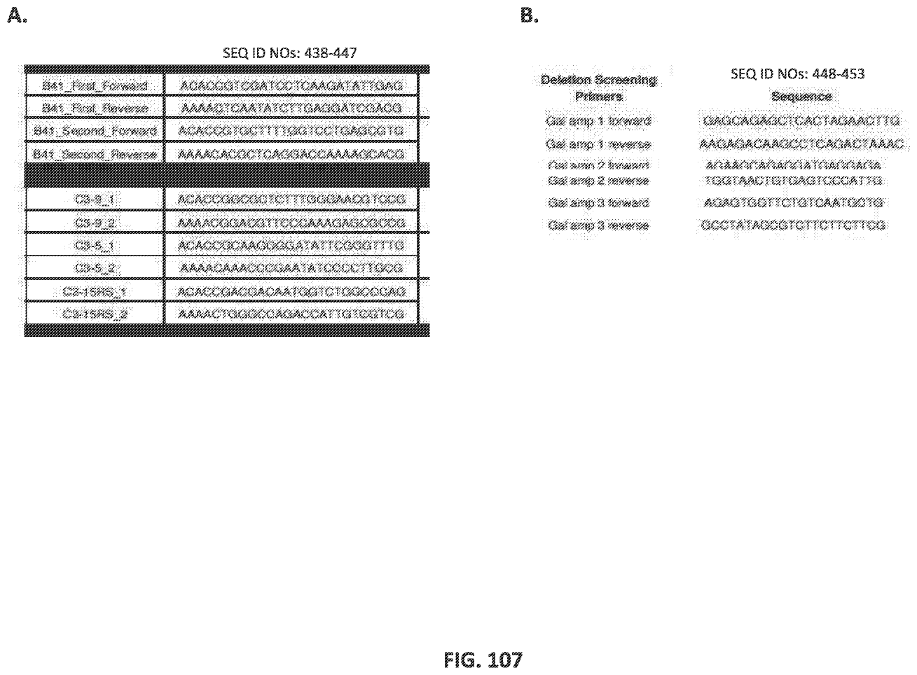

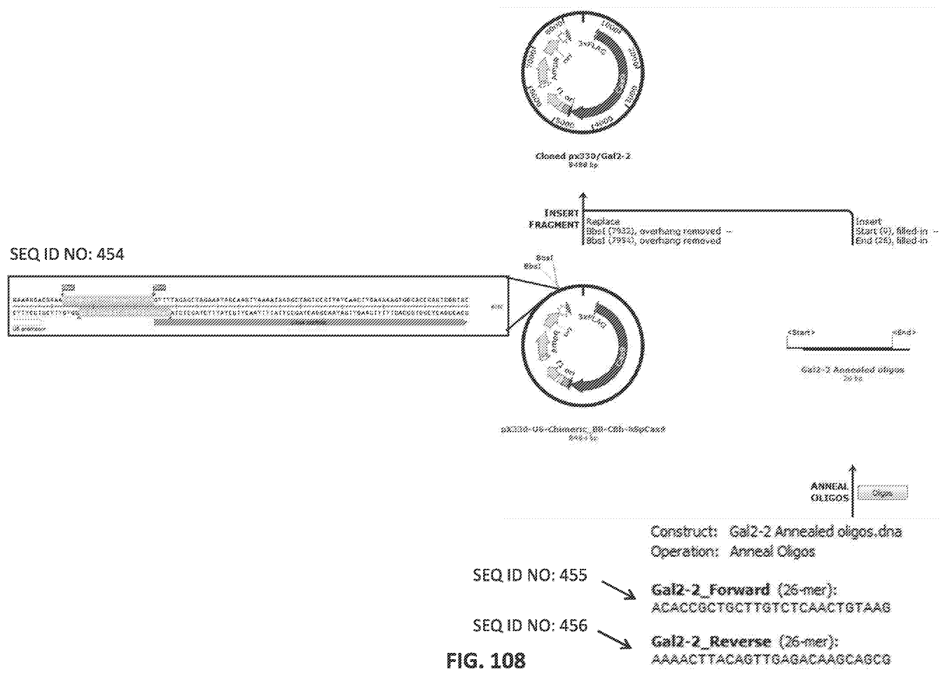

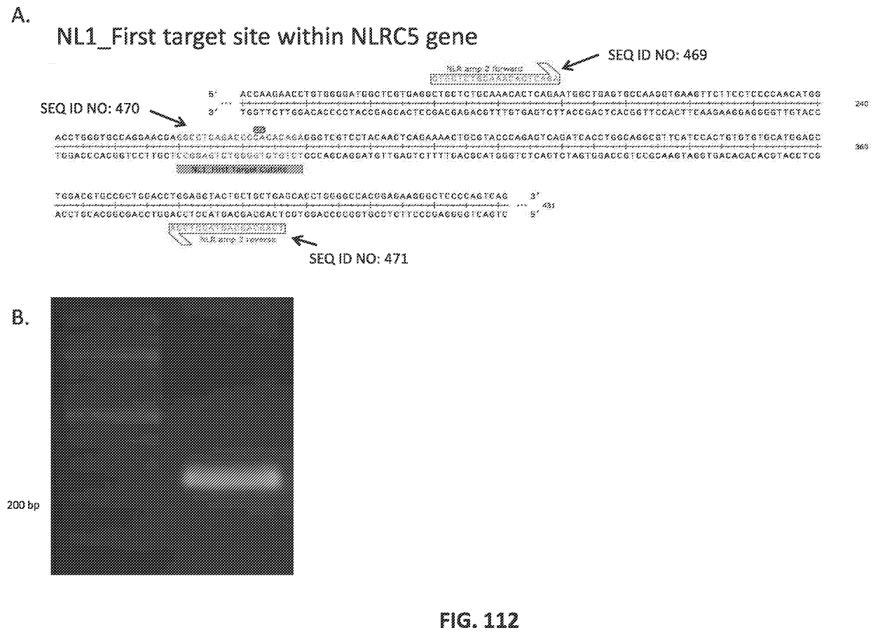

In some cases, a first exon of a gene is genetically modified. For example, one or more first exons of a gene that can be genetically modified can be a gene selected from a group consisting of NLRC5, TAP1, GGTA1, B4GALNT2, CMAH, CXCL10, MICA, MICB, C3, CIITA, and any combination thereof. For example, FIG. 112 A shows a guide RNA targeted a first exon of an NLCR5 gene. In other cases, a second exon of a gene is targeted. For example, FIG. 105, FIG. 106, and FIG. 107 show relevant sequences for primer pairs to generate first and second exon targeting guide RNAs as well as primer sequences to determine genetic modification by sequencing.

Transgenes that can be used and are specifically contemplated can include those genes that exhibit a certain identity and/or homology to genes disclosed herein, for example, ICP47, CD46, CD55, CD59, HLA-E, HLA-G (e.g., HLA-G1, HLA-G2, HLA-G3, HLA-G4, HLA-G5, HLA-G6, or HLA-G7), B2M, Spi9, PD-L1, PD-L2, CD47, galectin-9, any functional fragments thereof, and/or any combination thereof. Therefore, it is contemplated that if gene that exhibits at least or at least about 60%, 70%, 80%, 90%, 95%, 98%, or 99% homology, e.g., at least or at least about 99% to 90%; 90% to 80%; 80% to 70%; 70% to 60% homology; (at the nucleic acid or protein level), it can be used as a transgene. It is also contemplated that a gene that exhibits at least or at least about 60%, 65%, 70%, 75%, 80%, 85%, 90%, 95%, 98%, or 99%, identity e.g., at least or at least about 99% to 90%; 90% to 80%; 80% to 70%; 70% to 60% identity; (at the nucleic acid or protein level) can be used as a transgene.

A non-human animal can also comprise 1, 2, 3, 4, 5, 6, 7, 8, 9, 10, 11, 12, 13, 14, 15, 16, 17, 18, 19, 20, or more dominant negative transgenes. Expression of a dominant negative transgenes can suppress expression and/or function of a wild type counterpart of the dominant negative transgene. Thus, for example, a non-human animal comprising a dominant negative transgene X, can have similar phenotypes compared to a different non-human animal comprising an X gene whose expression is reduced. One or more dominant negative transgenes can be dominant negative NLRC5, dominant negative TAP1, dominant negative GGTA1, dominant negative CMAH, dominant negative B4GALNT2, dominant negative CXCL10, dominant negative MICA, dominant negative MICB, dominant negative CIITA, dominant negative C3, or any combination thereof.

Also provided is a non-human animal comprising one or more transgenes that encodes one or more nucleic acids that can suppress genetic expression, e.g., can knockdown a gene. RNAs that suppress genetic expression can comprise, but are not limited to, shRNA, siRNA, RNAi, and microRNA. For example, siRNA, RNAi, and/or microRNA can be given to a non-human animal to suppress genetic expression. Further, a non-human animal can comprise one or more transgene encoding shRNAs. shRNA can be specific to a particular gene. For example, a shRNA can be specific to any gene described in the application, including but not limited to, NLRC5, TAP1, GGTA1, B4GALNT2, CMAH, CXCL10, MICA, MICB, B4GALNT2, CIITA, C3, and/or any combination thereof.

When transplanted to a subject, cells, tissues, or organs from the genetically modified non-human animal can trigger lower immune responses (e.g., transplant rejection) in the subject compared to cells, tissues, or organs from a non-genetically modified counterpart. In some cases, the immune responses can include the activation, proliferation and cytotoxicity of T cells (e.g., CD8+ T cells and/or CD4+ T cells) and NK cells. Thus, phenotypes of genetically modified cells disclosed herein can be measured by co-culturing the cells with NK cells, T cells (e.g., CD8+ T cells or CD4+ T cells), and testing the activation, proliferation and cytotoxicity of the NK cells or T cells. In some cases, the T cells or NK cells activation, proliferation and cytotoxicity induced by the genetically modified cells can be lower than that induced by non-genetically modified cells. In some cases, phenotypes of genetically modified cells herein can be measured by Enzyme-Linked ImmunoSpot (ELISPOT) assays.

One or more transgenes can be from different species. For example, one or more transgenes can comprise a human gene, a mouse gene, a rat gene, a pig gene, a bovine gene, a dog gene, a cat gene, a monkey gene, a chimpanzee gene, or any combination thereof. For example, a transgene can be from a human, having a human genetic sequence. One or more transgenes can comprise human genes. In some cases, one or more transgenes are not adenoviral genes.

A transgene can be inserted into a genome of a non-human animal in a random or site-specific manner. For example, a transgene can be inserted to a random locus in a genome of a non-human animal. These transgenes can be fully functional if inserted anywhere in a genome. For instance, a transgene can encode its own promoter or can be inserted into a position where it is under the control of an endogenous promoter. Alternatively, a transgene can be inserted into a gene, such as an intron of a gene or an exon of a gene, a promoter, or a non-coding region. A transgene can be integrated into a first exon of a gene.

Sometimes, more than one copy of a transgene can be inserted into more than a random locus in a genome. For example, multiple copies can be inserted into a random locus in a genome. This can lead to increased overall expression than if a transgene was randomly inserted once. Alternatively, a copy of a transgene can be inserted into a gene, and another copy of a transgene can be inserted into a different gene. A transgene can be targeted so that it could be inserted to a specific locus in a genome of a non-human animal.

Expression of a transgene can be controlled by one or more promoters. A promoter can be a ubiquitous, tissue-specific promoter or an inducible promoter. Expression of a transgene that is inserted adjacent to a promoter can be regulated. For example, if a transgene is inserted near or next to a ubiquitous promoter, the transgene will be expressed in all cells of a non-human animal. Some ubiquitous promoters can be a CAGGS promoter, an hCMV promoter, a PGK promoter, an SV40 promoter, or a Rosa26 promoter.

A promoter can be endogenous or exogenous. For example, one or more transgenes can be inserted adjacent to an endogenous or exogenous Rosa26 promoter. Further, a promoter can be specific to a non-human animal. For example, one or more transgenes can be inserted adjacent to a porcine Rosa26 promoter.

Tissue specific promoter (which can be synonymous with cell-specific promoters) can be used to control the location of expression. For example, one or more transgenes can be inserted adjacent to a tissue-specific promoter. Tissue-specific promoters can be a FABP promoter, a Lck promoter, a CamKII promoter, a CD19 promoter, a Keratin promoter, an Albumin promoter, an aP2 promoter, an insulin promoter, an MCK promoter, an MyHC promoter, a WAP promoter, or a Col2A promoter. For example, a promoter can be a pancreas-specific promoter, e.g., an insulin promoter.

Inducible promoters can be used as well. These inducible promoters can be turned on and off when desired, by adding or removing an inducing agent. It is contemplated that an inducible promoter can be a Lac, tac, trc, trp, araBAD, phoA, recA, proU, cst-1, tetA, cadA, nar, PL, cspA, T7, VHB, Mx, and/or Trex.

A non-human animal or cells as described herein can comprise a transgene encoding insulin. A transgene encoding insulin can be a human gene, a mouse gene, a rat gene, a pig gene, a cattle gene, a dog gene, a cat gene, a monkey gene, a chimpanzee gene, or any other mammalian gene. For example, a transgene encoding insulin can be a human gene. A transgene encoding insulin can also be a chimeric gene, for example, a partially human gene.

Expression of transgenes can be measured by detecting the level of transcripts of the transgenes. For example, expression of transgenes can be measured by Northern blotting, nuclease protection assays (e.g., RNase protection assays), reverse transcription PCR, quantitative PCR (e.g., real-time PCR such as real-time quantitative reverse transcription PCR), in situ hybridization (e.g., fluorescent in situ hybridization (FISH)), dot-blot analysis, differential display, Serial analysis of gene expression, subtractive hybridization, microarrays, nanostring, and/or sequencing (e.g., next-generation sequencing). In some cases, expression of transgenes can be measured by detecting proteins encoded by the genes. For example, expression of one or more genes can be measured by protein immunostaining, protein immunoprecipitation, electrophoresis (e.g., SDS-PAGE), Western blotting, bicinchoninic acid assay, spectrophotometry, mass spectrometry, enzyme assays (e.g., enzyme-linked immunosorbent assays), immunohistochemistry, flow cytometry, and/or immunocytochemistry. In some cases, expression of transgenes can be measured by microscopy. The microscopy can be optical, electron, or scanning probe microscopy. In some cases, optical microscopy comprises use of bright field, oblique illumination, cross-polarized light, dispersion staining, dark field, phase contrast, differential interference contrast, interference reflection microscopy, fluorescence (e.g., when particles, e.g., cells, are immunostained), confocal, single plane illumination microscopy, light sheet fluorescence microscopy, deconvolution, or serial time-encoded amplified microscopy.

Insertion of transgenes can be validated by genotyping. Methods for genotyping can include sequencing, restriction fragment length polymorphism identification (RFLPI), random amplified polymorphic detection (RAPD), amplified fragment length polymorphism detection (AFLPD), PCR (e.g., long range PCR, or stepwise PCR), allele specific oligonucleotide (ASO) probes, and hybridization to DNA microarrays or beads. In some cases, genotyping can be performed by sequencing. In some cases, sequencing can be high fidelity sequencing. Methods of sequencing can include Maxam-Gilbert sequencing, chain-termination methods (e.g., Sanger sequencing), shotgun sequencing, and bridge PCR. In some cases, genotyping can be performed by next-generation sequencing. Methods of next-generation sequencing can include massively parallel signature sequencing, colony sequencing, pyrosequencing (e.g., pyrosequencing developed by 454 Life Sciences), single-molecule rea-time sequencing (e.g., by Pacific Biosciences), Ion semiconductor sequencing (e.g., by Ion Torrent semiconductor sequencing), sequencing by synthesis (e.g., by Solexa sequencing by Illumina), sequencing by ligation (e.g., SOLiD sequencing by Applied Biosystems), DNA nanoball sequencing, and heliscope single molecule sequencing. In some cases, genotyping of a non-human animal herein can comprise full genome sequencing analysis.

In some cases, insertion of a transgene in an animal can be validated by sequencing (e.g., next-generation sequencing) a part of the transgene or the entire transgene. For example, insertion of a transgene adjacent to a Rosa26 promoter in a pig can be validated by next generation sequencing of Rosa exons 1 to 4, e.g., using the forward primer 5′-cgcctagagaagaggctgtg-3′ (SEQ ID No. 35), and reverse primer 5′-ctgctgtggctgtggtgtag-3′ (SEQ ID No. 36).

TABLE 2
cDNA sequences of exemplary transgenes*

| SEQ ID | | |
|---|---|---|
| No. | Gene | Accession No. |
| 37 | CD46 | NM_213888 |
| 38 | CD55 | AF228059.1 |
| 39 | CD59 | AF020302 |
| 40 | ICP47 | EU445532.1 |
| 41 | HLA-G1 | NM_002127.5 |
| 42 | HLA-E | NM_005516.5 |
| 43 | Human β-2- | NM_004048.2 |
| | microglobulin | |
| 44 | Human PD-L1 | NM_001267706.1 |
| 45 | Human PD-L2 | NM_025239.3 |
| 46 | Human Spi9 | NM_004155.5 |
| 47 | Human CD47 | NM_001777.3 |
| 48 | Human galectin-9 | NM_009587.2 |

*The sequences for Table 2 can be found in Table 18.

TABLE 3
Sequences of proteins encoded by exemplary transgenes*

| SEQ ID | | |
|---|---|---|
| No. | Protein | Accession No. |
| 49 | CD46 | NP_999053.1 |
| 50 | CD55 | AAG14412.1 |
| 51 | CD59 | AAC67231.1 |
| 52 | ICP47 | ACA28836.1 |
| 53 | HLA-G1 | NP_002118.1 |
| 54 | HLA-E | NP_005507.3 |
| 55 | Human β-2- | NP_004039.1 |
| | microglobulin | |
| 56 | Human PD-L1 | NP_001254635.1 |
| 57 | Human PD-L2 | NP_079515.2 |
| 58 | Human Spi9 | NP_004146.1 |
| 59 | Human CD47 | NP_001768.1 |
| 60 | Human galectin-9 | NP_033665.1 |

*The sequences for Table 3 can be found in Table 18.

Populations of Non-Human Animals

Provided herein is a single non-human animal and also a population of non-human animals. A population of non-human animals can be genetically identical. A population of non-human animals can also be phenotypical identical. A population of non-human animals can be both phenotypical and genetically identical.

Further provided herein is a population of non-human animals, which can be genetically modified. For example, a population can comprise at least or at least about 2, 5, 10, 50, 100, or 200, non-human animals as disclosed herein. The non-human animals of a population can have identical phenotypes. For example, the non-human animals of a population can be clones. A population of non-human animal can have identical physical characteristics. The non-human animals of a population having identical phenotypes can comprise a same transgene(s). The non-human animals of a population having identical phenotypes can also comprise a same gene(s) whose expression is reduced. The non-human animals of a population having identical phenotypes can also comprise a same gene(s) whose expression is reduced and comprise a same transgene(s). A population of non-human animals can comprise at least or at least about 2, 5, 10, 50, 100, or 200, non-human animals having identical phenotypes. For example, the phenotypes of any particular litter can have the identical phenotype (e.g., in one example, anywhere from 1 to about 20 non-human animals). The non-human animals of a population can be pigs having identical phenotypes.

The non-human animals of a population can have identical genotypes. For example, all nucleic acid sequences in the chromosomes of non-human animals in a population can be identical. The non-human animals of a population having identical genotypes can comprise a same transgene(s). The non-human animals of a population having identical genotypes can also comprise a same gene(s) whose expression is reduced. The non-human animals of a population having identical genotypes can also comprise a same gene(s) whose expression is reduced and comprise a same transgene(s). A population of non-human animals can comprise at least or at least about 2, 5, 50, 100, or 200 non-human animals having identical genotypes. The non-human animals of a population can be pigs having identical genotypes.

Cells from two or more non-human animals with identical genotypes and/or phenotypes can be used in a tolerizing vaccine. In some cases, a tolerizing vaccine disclosed herein can comprise a plurality of the cells (e.g., genetically modified cells) from two or more non-human animals (e.g., pigs) with identical genotypes and/or phenotypes. A method for immunotolerizing a recipient to a graft can comprise administering to the recipient a tolerizing vaccine comprising a plurality of cells (e.g., genetically modified cells) from two or more non-human animals with identical genotypes or phenotypes.

Cells from two or more non-human animals with identical genotypes and/or phenotypes can be used in transplantation. In some cases, a graft (e.g., xenograft or allograft) can comprise a plurality of cells from two or more non-human animals with identical genotypes and/or phenotypes. In embodiments of the methods described herein, e.g., a method for treating a disease in a subject in need thereof, can comprise transplanting a plurality of cells (e.g., genetically modified cells) from two or more non-human animals with identical genotypes and/or phenotypes.

Populations of non-human animals can be generated using any method known in the art. In some cases, populations of non-human animals can be generated by breeding. For example, inbreeding can be used to generate a phenotypically or genetically identical non-human animal or population of non-human animals. Inbreeding, for example, sibling to sibling or parent to child, or grandchild to grandparent, or great grandchild to great grandparent, can be used. Successive rounds of inbreeding can eventually produce a phenotypically or genetically identical non-human animal. For example, at least or at least about 2, 3, 4, 5, 10, 20, 30, 40, or 50 generations of inbreeding can produce a phenotypically and/or a genetically identical non-human animal. It is thought that after 10-20 generations of inbreeding, the genetic make-up of a non-human animal is at least 99% pure. Continuous inbreeding can lead to a non-human animal that is essentially isogenic, or close to isogenic as a non-human animal can be without being an identical twin.

Breeding can be performed using non-human animals that have the same genotype. For example, the non-human animals have the same gene(s) whose expression is reduced and/or carry the same transgene(s). Breeding can also be performed using non-human animals having different genotypes. Breeding can be performed using a genetically modified non-human animal and non-genetically modified non-human animal, for example, a genetically modified female pig and a wild-type male pig, or a genetically modified male pig and a wild-type female pig. All these combinations of breeding can be used to produce a non-human animal of desire.

Populations of genetically modified non-human animals can also be generated by cloning. For example, the populations of genetically modified non-human animal cells can be asexually producing similar populations of genetically or phenotypically identical individual non-human animals. Cloning can be performed by various methods, such as twinning (e.g., splitting off one or more cells from an embryo and grow them into new embryos), somatic cell nuclear transfer, or artificial insemination. More details of the methods are provided throughout the disclosure.

II. Genetically Modified Cells

Disclosed herein are one or more genetically modified cells that can be used to treat or prevent disease. These genetically modified cells can be from genetically modified non-human animals. For example, genetically modified non-human animals as disclosed above can be processed so that one or more cells are isolated to produce isolated genetically modified cells. These isolated cells can also in some cases be further genetically modified cells. However, a cell can be modified ex vivo, e.g., outside an animal using modified or non-modified human or non-human animal cells. For example, cells (including human and non-human animal cells) can be modified in culture. It is also contemplated that a genetically modified cell can be used to generate a genetically modified non-human animal described herein. In some cases, the genetically modified cell can be isolated from a genetically modified animal. In some cases, the genetically modified cell can be derived from a cell from a non-genetically modified animal. Isolation of cells can be performed by methods known in the art, including methods of primary cell isolation and culturing. It is specifically contemplated that a genetically modified cell is not extracted from a human.

Therefore, anything that can apply to the genetically modified non-human animals including the various methods of making as described throughout can also apply herein. For example, all the genes that are disrupted and the transgenes that are overexpressed are applicable in making genetically modified cells used herein. Further, any methods for testing the genotype and expression of genes in the genetically modified non-human animals described throughout can be used to test the genetic modification of the cells.

A genetically modified cell can be from a member of the Laurasiatheria superorder or a non-human primate. Such genetically modified cell can be isolated from a member of the Laurasiatheria superorder or a non-human primate. Alternatively, such genetically modified cell can be originated from a member of the Laurasiatheria superorder or a non-human primate. For example, the genetically modified cell can be made from a cell isolated from a member of the Laurasiatheria superorder or a non-human primate, e.g., using cell culturing or genetic modification methods.

Genetically modified cells, e.g., cells from a genetically modified animal or cells made ex vivo, can be analyzed and sorted. In some cases, genetically modified cells can be analyzed and sorted by flow cytometry, e.g., fluorescence-activated cell sorting. For example, genetically modified cells expressing a transgene can be detected and purified from other cells using flow cytometry based on a label (e.g., a fluorescent label) recognizing the polypeptide encoded by the transgene.

In some cases, genetically modified cells can reduce, inhibit, or eliminate an immune response. For example, a genetic modification can decrease cellular effector function, decrease proliferation, decrease, persistence, and/or reduce expression of cytolytic effector molecules such as Granzyme B and CD107alpha in an immune cell. An immune cell can be a monocyte and/or macrophage. In some cases, T cell-derived cytokines, such as IFN-g, can activate macrophages via secretion of IFN-gamma. In some cases, T cell activation is inhibited and may cause a macrophage to also be inhibited.

Stem cells, including, non-human animal and human stem cells can be used. Stem cells do not have the capability to generating a viable human being. For example, stem cells can be irreversibly differentiated so that they are unable to generate a viable human being. Stem cells can be pluripotent, with the caveat that the stem cells cannot generate a viable human.

As discussed above in the section regarding the genetically modified non-human animals, the genetically modified cells can comprise one or more genes whose expression is reduced. The same genes as disclosed above for the genetically modified non-human animals can be disrupted. For example, a genetically modified cell comprising one or more genes whose expression is disrupted, e.g., reduced, where the one or more genes comprise NLRC5, TAP1, GGTA1, B4GALNT2, CMAH, CXCL10, MICA, MICB, C3, CIITA and/or any combination thereof. Further, the genetically modified cell can comprise one or more transgenes comprising one or more polynucleotide inserts. For example, a genetically modified cell can comprise one or more transgenes comprising one or more polynucleotide inserts of ICP47, CD46, CD55, CD 59, HLA-E, HLA-G (e.g., HLA-G1, HLA-G2, HLA-G3, HLA-G4, HLA-G5, HLA-G6, or HLA-G7), B2M, Spi9, PD-L1, PD-L2, CD47, galectin-9, any functional fragments thereof, or any combination thereof. A genetically modified cell can comprise one or more reduced genes and one or more transgenes. For example, one or more genes whose expression is reduced can comprise any one of NLRC5, TAP1, GGTA1, B4GALNT2, CMAH, CXCL10, MICA, MICB, CIITA, and/or any combination thereof, and one or more transgene can comprise ICP47, CD46, CD55, CD 59, HLA-E, HLA-G (e.g., HLA-G1, HLA-G2, HLA-G3, HLA-G4, HLA-G5, HLA-G6, or HLA-G7), B2M, Spi9, PD-L1, PD-L2, CD47, galectin-9, any functional fragments thereof, and/or any combination thereof. In some cases, a genetically modified cell can comprise reduced expression of NLRC5, C3, GGTA1, CMAH, and B4GALNT2, and transgenes comprising polynucleotides encoding proteins or functional fragments thereof, where the proteins comprise HLA-G1, Spi9, PD-L1, PD-L2, CD47, and galectin-9. In some cases, a genetically modified cell can comprise reduced expression of TAP1, C3, GGTA1, CMAH, and B4GALNT2, and transgenes comprising polynucleotides encoding proteins or functional fragments thereof, where the proteins comprise HLA-G1, Spi9, PD-L1, PD-L2, CD47, and galectin-9. In some cases, a genetically modified cell can comprise reduced expression of NLRC5, TAP1, C3, GGTA1, CMAH, and B4GALNT2, and transgenes comprising polynucleotides encoding proteins or functional fragments thereof, where the proteins comprise HLA-G1, Spi9, PD-L1, PD-L2, CD47, and galectin-9. In some cases, CD47, PD-L1, and PD-L2 encoded by the transgenes herein can be human CD47, human PD-L1 and human PDO-L2. In some cases, the genetically modified cell can be coated with CD47 on its surface. Coating of CD47 on the surface of a cell can be accomplished by biotinylating the cell surface followed by incubating the biotinylated cell with a streptavidin-CD47 chimeric protein. The coated CD47 can be human CD47.

As discussed above in the section regarding the genetically modified non-human animals, the genetically modified cell can comprise 1, 2, 3, 4, 5, 6, 7, 8, 9, 10, 11, 12, 13, 14, 15, 16, 17, 18, 19, 20 or more disrupted genes. A genetically modified cell can also comprise 1, 2, 3, 4, 5, 6, 7, 8, 9, 10, 11, 12, 13, 14, 15, 16, 17, 18, 19, 20, or more transgenes.

As discussed in detail above, a genetically modified cell, e.g., porcine cell, can also comprise dominant negative transgenes and/or transgenes expressing one or more knockdown genes. Also as discussed above, expression of a transgene can be controlled by one or more promoters.

A genetically modified cell can be one or more cells from tissues or organs, the tissues or organs including brain, lung, liver, heart, spleen, pancreas, small intestine, large intestine, skeletal muscle, smooth muscle, skin, bones, adipose tissues, hairs, thyroid, trachea, gall bladder, kidney, ureter, bladder, aorta, vein, esophagus, diaphragm, stomach, rectum, adrenal glands, bronchi, ears, eyes, retina, genitals, hypothalamus, larynx, nose, tongue, spinal cord, or ureters, uterus, ovary and testis. For example, a genetically modified cell, e.g., porcine cell, can be from brain, heart, liver, skin, intestine, lung, kidney, eye, small bowel, or pancreas. In some cases, a genetically modified cell can be from a pancreas. More specifically, pancreas cells can be islet cells. Further, one or more cells can be pancreatic α cells, pancreatic β cells, pancreatic δ cells, pancreatic F cells (e.g., PP cells), or pancreatic ε cells. For example, a genetically modified cell can be pancreatic β cells. Tissues or organs disclosed herein can comprise one or more genetically modified cells. The tissues or organs can be from one or more genetically modified animals described in the application, e.g., pancreatic tissues such as pancreatic islets from one or more genetically modified pigs.

A genetically modified cell, e.g., porcine cell, can comprise one or more types of cells, where the one or more types of cells include Trichocytes, keratinocytes, gonadotropes, corticotropes, thyrotropes, somatotropes, lactotrophs, chromaffin cells, parafollicular cells, glomus cells melanocytes, nevus cells, Merkel cells, odontoblasts, cementoblasts corneal keratocytes, retina Muller cells, retinal pigment epithelium cells, neurons, glias (e.g., oligodendrocyte astrocytes), ependymocytes, pinealocytes, pneumocytes (e.g., type I pneumocytes, and type II pneumocytes), clara cells, goblet cells, G cells, D cells, ECL cells, gastric chief cells, parietal cells, foveolar cells, K cells, D cells, I cells, goblet cells, paneth cells, enterocytes, microfold cells, hepatocytes, hepatic stellate cells (e.g., Kupffer cells from mesoderm), cholecystocytes, centroacinar cells, pancreatic stellate cells, pancreatic α cells, pancreatic β cells, pancreatic δ cells, pancreatic F cells (e.g., PP cells), pancreatic ε cells, thyroid (e.g., follicular cells), parathyroid (e.g., parathyroid chief cells), oxyphil cells, urothelial cells, osteoblasts, osteocytes, chondroblasts, chondrocytes, fibroblasts, fibrocytes, myoblasts, myocytes, myosatellite cells, tendon cells, cardiac muscle cells, lipoblasts, adipocytes, interstitial cells of cajal, angioblasts, endothelial cells, mesangial cells (e.g., intraglomerular mesangial cells and extraglomerular mesangial cells), juxtaglomerular cells, macula densa cells, stromal cells, interstitial cells, telocytes simple epithelial cells, podocytes, kidney proximal tubule brush border cells, sertoli cells, leydig cells, granulosa cells, peg cells, germ cells, spermatozoon ovums, lymphocytes, myeloid cells, endothelial progenitor cells, endothelial stem cells, angioblasts, mesoangioblasts, and pericyte mural cells. A genetically modified cell can potentially be any cells used in cell therapy. For example, cell therapy can be pancreatic β cells supplement or replacement to a disease such as diabetes.

A genetically modified cell, e.g., porcine cell, can be from (e.g., extracted from) a non-human animal. One or more cells can be from a mature adult non-human animal. However, one or more cells can be from a fetal or neonatal tissue.

Depending on the disease, one or more cells can be from a transgenic non-human animal that has grown to a sufficient size to be useful as an adult donor, e.g., an islet cell donor. In some cases, non-human animals can be past weaning age. For example, non-human animals can be at least or at least about six months old. In some cases, non-human animals can be at least or at least about 18 months old. A non-human animal in some cases, survive to reach breeding age. For example, islets for xenotransplantation can be from neonatal (e.g., age 3-7 days) or preweaning (e.g., age 14 to 21 days) donor pigs. One or more genetically modified cells, e.g., porcine cells, can be cultured cells. For example, cultured cells can be from wild-type cells or from genetically modified cells (as described herein). Furthermore, cultured cells can be primary cells. Primary cells can be extracted and frozen, e.g., in liquid nitrogen or at −20° C. to −80° C. Cultured cells can also be immortalized by known methods, and can be frozen and stored, e.g., in liquid nitrogen or at −20° C. to −80° C.

Genetically modified cells, e.g., porcine cells, as described herein can have a lower risk of rejection, when compared to when a wild-type non-genetically modified cell is transplanted.

Disclosed herein is a vector comprising a polynucleotide sequence of ICP47, CD46, CD55, CD59, HLA-E, HLA-G (e.g., HLA-G1, HLA-G2, HLA-G3, HLA-G4, HLA-G5, HLA-G6, or HLA-G7), B2M, Spi9, PD-L1, PD-L2, CD47, galectin-9, any functional fragments thereof, or any combination thereof. These vectors can be inserted into a genome of a cell (by transfection, transformation, viral delivery, or any other known method). These vectors can encode ICP47, CD46, CD55, CD59, HLA-E, HLA-G (e.g., HLA-G1, HLA-G2, HLA-G3, HLA-G4, HLA-G5, HLA-G6, or HLA-G7), B2M Spi9, PD-L1, PD-L2, CD47, and/or galectin-9 proteins or functional fragments thereof.

Vectors contemplated include, but not limited to, plasmid vectors, artificial/mini-chromosomes, transposons, and viral vectors. Further disclosed herein is an isolated or synthetic nucleic acid comprising an RNA, where the RNA is encoded by any sequence in Table 2. RNA can also encode for any sequence that exhibits at least or at least about 50%, 60%, 70%, 80%, 90%, 95%, 99%, or 100% homology to any sequence in Table 2. RNA can also encode for any sequence that exhibits at least or at least about 50%, 60%, 70%, 80%, 90%, 95%, 99%, or 100% identity to any sequence in Table 2.

RNA can be a single-chain guide RNA. The disclosure also provides an isolated or synthesized nucleic acid comprising any sequence in Table 1. RNA can also provide an isolated or synthesized nucleic acid that exhibits at least or at least about 50%, 60%, 70%, 80%, 90%, 95%, 99%, or 100% homology to any sequence in Table 1. RNA can also provide an isolated or synthesized nucleic acid that exhibits at least or at least about 50%, 60%, 70%, 80%, 90%, 95%, 99%, or 100% identity to any sequence in Table 1.

Guide RNA sequences can be used in targeting one or more genes in a genome of a non-human animal. For example, guide RNA sequence can target a single gene in a genome of non-human animal. In some cases, guide RNA sequences can target one or more target sites of each of one or more genes in a genome of a non-human animal.

Genetically modified cells can also be leukocytes, lymphocytes, B lymphocytes, or any other cell such as islet cells, islet beta cells, or hepatocytes. These cells can be fixed or made apopototic by any method disclosed herein, e.g., by ECDI fixation.

A genetically modified cells can be derived (e.g., retrieved) from a non-human fetal animal, perinatal non-human animal, neonatal non-human animal, preweaning non-human animal, young adult non-human animal, adult non-human animal, or any combination thereof. In some cases, a genetically modified non-human animal cell can be derived from an embryonic tissue, e.g., an embryonic pancreatic tissue. For example, a genetically modified cell can be derived (e.g., retrieved) from an embryonic pig pancreatic tissue from embryonic day 42 (E42).

The term “fetal animal” and its grammatical equivalents can refer to any unborn offspring of an animal. The term “perinatal animal” and its grammatical equivalents can refer to an animal immediately before or after birth. For example, a perinatal period can start from 20th to 28th week of gestation and ends 1 to 4 weeks after birth. The term “neonatal animal” and its grammatical equivalents can refer to any new born animals. For example, a neonatal animal can be an animal born within a month. The term “preweaning non-human animal” and its grammatical equivalents can refer to any animal before being withdrawn from the mother's milk.

Genetically modified non-human animal cells can be formulated into a pharmaceutical composition. For example, the genetically modified non-human animal cells can be combined with a pharmaceutically acceptable excipient. An excipient that can be used is saline. The pharmaceutical composition can be used to treat patients in need of transplantation.

A genetically modified cell can comprise reduced expression of any genes, and/or any transgenes disclosed herein. Genetic modification of the cells can be done by using any of the same method as described herein for making the genetically modified animals. In some cases, a method of making a genetically modified cell originated from a non-human animal can comprise reducing expression of one or more genes and/or inserting one or more transgenes. The reduction of gene expression and/or transgene insertion can be performed using any methods described in the application, e.g., gene editing.

Genetically Modified Cells Derived from Stem Cells

Genetically modified cells can be a stem cell. These genetically modified stem cells can be used to make a potentially unlimited supply of cells that can be subsequently processed into fixed or apoptotic cells by the methods disclosed herein. As discussed above, stem cells are not capable of generating a viable human being.

The production of hundreds of millions of insulin-producing, glucose-responsive pancreatic beta cells from human pluripotent stem cells provides an unprecedented cell source for cell transplantation therapy in diabetes (Pagliuca et al., 2014). Other human stem cell-(embryonic, pluripotent, placental, induced pluripotent, etc.) derived cell sources for cell transplantation therapy in diabetes and in other diseases are being developed.

These stem cell-derived cellular grafts are subject to rejection. The rejection can be mediated by CD8+ T cells. In Type 1 diabetic recipients, human stem cell-derived functional beta cells are subject to rejection and autoimmune recurrence. Both are thought to be mediated by CD8+ T cells.

To interfere with activation and effector function of these allo-reactive and auto-reactive CD8+ T cells, established molecular methods of gene modification, including CRISP/Cas9 gene targeting, can be used to mutate the NLRC5, TAP1, and/or B2M genes in human stem cells for the purpose of preventing cell surface expression of functional MHC class I in the stem cell-derived, partially or fully differentiated cellular graft. Thus, transplanting human stem cell-derived cellular grafts lacking functional expression of MHC class I can minimize the requirements of immunosuppression otherwise required to prevent rejection and autoimmune recurrence.

However, lack of MHC class I expression on transplanted human cells will likely cause the passive activation of natural killer (NK) cells (Ohlen et al, 1989). NK cell cytotoxicity can be overcome by the expression of the human MHC class 1 gene, HLA-E, which stimulates the inhibitory receptor CD94/NKG2A on NK cells to prevent cell killing (Weiss et al., 2009; Lilienfeld et al., 2007; Sasaki et al., 1999). Successful expression of the HLA-E gene was dependent on co-expression of the human B2M (beta 2 microglobulin) gene and a cognate peptide (Weiss et al., 2009; Lilienfeld et al., 2007; Sasaki et al., 1999; Pascasova et al., 1999). A nuclease mediated break in the stem cell DNA allows for the insertion of one or multiple genes via homology directed repair. The HLA-E and hB2M genes in series can be integrated in the region of the nuclease mediated DNA break thus preventing expression of the target gene (for example, NLRC5) while inserting the transgenes.

To further minimize, if not eliminate, the need for maintenance immunosuppression in recipients of stem cell derived cellular grafts lacking functional expression of MHC class I, recipients of these grafts can also be treated with tolerizing apoptotic donor cells disclosed herein.

The methods for the production of insulin-producing pancreatic beta cells (Pagliuca et al., 2014) can potentially be applied to non-human (e.g., pig) primary isolated pluripotent, embryonic stem cells or stem-like cells (Goncalves et al., 2014; Hall et al. V. 2008). However, the recipient of these insulin-producing pancreatic beta cells likely has an active immune response that threatens the success of the graft. To overcome antibody-mediated and CD8+ T cell immune attack, the donor animal can be genetically modified before isolation of primary non-human pluripotent, embryonic stem cells or stem-like cells to prevent the expression of the GGTA1, CMAH, B4GalNT2, or MHC class I-related genes as disclosed throughout the application. The pluripotent, embryonic stem cells or stem-like cells isolated from genetically modified animals could then be differentiated into millions of insulin-producing pancreatic beta cells.

Xenogeneic stem cell-derived cell transplants can be desirable in some cases. For example, the use of human embryonic stem cells may be ethically objectionable to the recipient. Therefore, human recipients may feel more comfortable receiving a cellular graft derived from non-human sources of embryonic stem cells.

Non-human stem cells may include pig stem cells. These stem cells can be derived from wild-type pigs or from genetically engineered pigs. If derived from wild-type pigs, genetic engineering using established molecular methods of gene modification, including CRISP/Cas9 gene targeting, may best be performed at the stem cell stage. Genetic engineering may be targeted to disrupt expression of NLRC5, TAP1, and/or B2M genes to prevent functional expression of MHC class I. Disrupting genes such as NLRC5, TAP1, and B2M in the grafts can cause lack of functional expression of MHC class I on graft cells including on islet beta cells, thereby interfering with the post-transplant activation of autoreactive CD8+ T cells. Thus, this can protect the transplant, e.g., transplanted islet beta cells, from the cytolytic effector functions of autoreactive CD8+ T cells.

However, as genetic engineering of stem cells may alter their potential for differentiation, an approach can be to generate stem cell lines from genetically engineered pigs, including those pigs, in whom the expression of NLRC5, TAP1, and/or B2M genes has been disrupted.

Generation of stem cells from pigs genetically modified to prevent the expression also of the GGTA1, CMAH, B4GalNT2 genes or modified to express transgenes that encode for complement regulatory proteins CD46, CD55, or CD59, as disclosed throughout the application, could further improve the therapeutic use of the insulin-producing pancreatic beta cells or other cellular therapy products. Likewise, the same strategy as described herein can be used in other methods and compositions described throughout.

Like in recipients of human stem cell-derived cellular grafts lacking functional expression of MHC class I, the need for maintenance immunosuppression in recipients of pig stem cell-derived grafts can be further minimized by peritransplant treatments with tolerizing apoptotic donor cells.

III. Tolerizing Vaccines

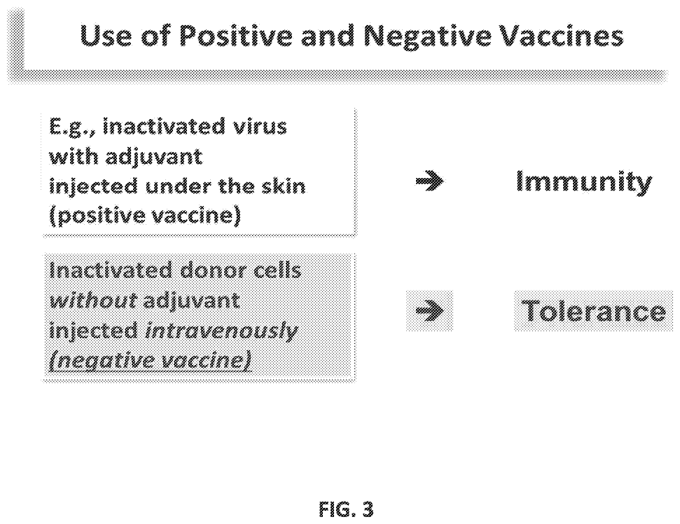

Traditionally, vaccines are used to confer immunity to a host. For example, injecting an inactivated virus with adjuvant under the skin can lead to temporary or permanent immunity to the active and/or virulent version of the virus. This can be referred to as a positive vaccine (FIG. 3). However, inactivated cells (e.g., cells from a donor or an animal genetically different from the donor) that are injected intravenously can result in tolerance of donor cells or cells with similar cellular markers. This can be referred to as a tolerizing vaccine (also referred to as a negative vaccine) (FIG. 3). The inactive cells can be injected without an adjuvant. Alternatively, the inactive cells can be injected with an adjuvant. These tolerizing vaccines can be advantageous in transplantation, for example, in xenotransplantation, by tolerizing a recipient and preventing rejection. Tolerization can be conferred to a recipient without the use of immunosuppressive therapies. However, in some cases, other immunosuppressive therapies in combination with tolerizing vaccines can decrease transplantation rejection.

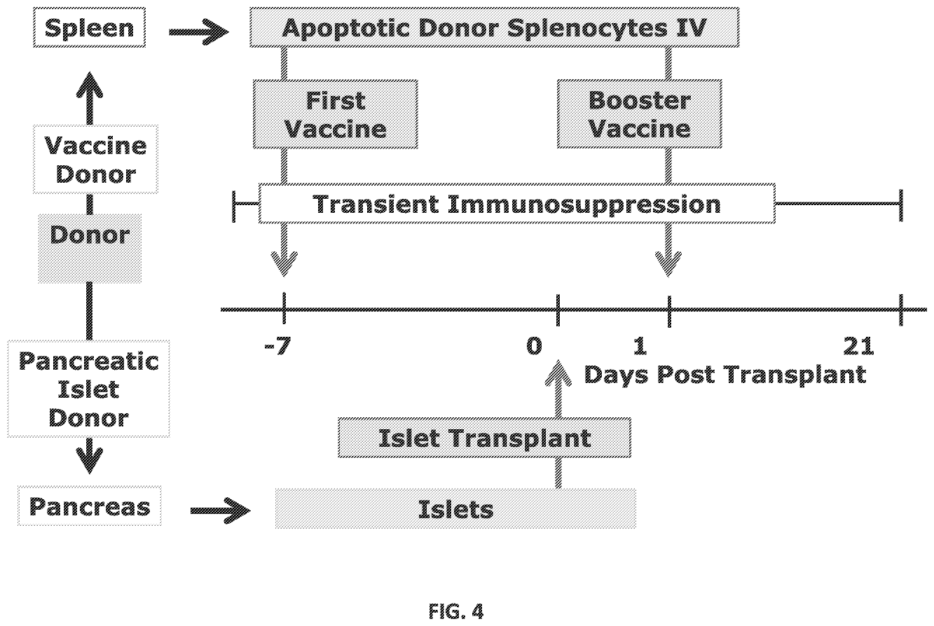

FIG. 4 demonstrates an exemplary approach to extending the survival of transplanted grafts (e.g., xenografts) in a subject (e.g., a human or a non-human primate) with infusion (e.g., intravenous infusion) of apoptotic cells from the donor for tolerizing vaccination under the cover of transient immunosuppression. A donor can provide xenografts for transplantation (e.g., islets), as well as cells (e.g., splenocytes) as a tolerizing vaccine. The tolerizing vaccine cells can be apoptotic cells (e.g., by ECDI fixation) and administered to the recipient before (e.g., the first vaccine, on day 7 before the transplantation) and after the transplantation (e.g., the booster vaccine, on day 1 after the transplantation). The tolerizing vaccine can provide transient immunosuppression that extends the time of survival of the transplanted grafts (e.g., islets).

Tolerizing vaccines can comprise one or more of the following types of cells: i) apoptotic cells comprising genotypically identical cells with reduced expression of GGTA1 alone, or GGTA1 and CMAH, or GGTA1, CMAH, and B4GALNT2. This can minimize or eliminate cell-mediated immunity and cell-dependent antibody-mediated immunity to organ, tissue, cell, and cell line grafts (e.g., xenografts) from animals that are genotypically identical with the apoptotic cell vaccine donor animal, or from animals that have undergone additional genetic modifications (e.g., suppression of NLRC5, TAP1, MICA, MICB, CXCL10, C3, CIITA genes or expression of transgenes comprising two or more polynucleotide inserts of ICP47, CD46, CD55, HLA-E, HLA-G (e.g., HLA-G1, HLA-G2, HLA-G3, HLA-G4, HLA-G5, HLA-G6, or HLA-G7), B2M, CD59, or any functional fragments thereof), but are genotypically similar to the donor animal from which the apoptotic cell vaccine is derived; ii) apoptotic stem cell (e.g., embryonic, pluripotent, placental, induced pluripotent, etc.)-derived donor cells (e.g., leukocytes, lymphocytes, T lymphocytes, B lymphocytes, red blood cells, graft cells, or any other donor cell) for minimizing or eliminating cell-mediated immunity and cell-dependent antibody-mediated immunity to organ, tissue, cell, and cell line grafts (e.g., xenografts) from animals that are genotypically identical with the apoptotic cell vaccine donor animal or from animals that have undergone additional genetic modifications (e.g., suppression of NLRC5, TAP1, MICA, MICB, CXCL10, C3, CIITA genes or expression of transgenes comprising two or more polynucleotide inserts of ICP47, CD46, CD55, HLA-E, HLA-G (e.g., HLA-G1, HLA-G2, HLA-G3, HLA-G4, HLA-G5, HLA-G6, or HLA-G7), B2M, CD59, or any functional fragments thereof), but are genotypically similar to the donor animal from which the apoptotic stem cell-derived cell vaccine is derived; iii) apoptotic stem cell (e.g., embryonic, pluripotent, placental, induced pluripotent, etc.)-derived donor cells (leukocytes, lymphocytes, T lymphocytes, B lymphocytes, red blood cells, graft cells such as functional islet beta cells, or any other donor cell) for minimizing or eliminating cell-mediated immunity and cell-dependent antibody-mediated immunity to organ, tissue, cell, and cell grafts (e.g., allografts) that are genotypically identical with the human stem cell line or to grafts (e.g., allografts) derived from the same stem cell line that have undergone genetic modifications (e.g., suppression of NLRC5, TAP1, MICA, MICB, CXCL10, C3, CIITA genes) but are otherwise genotypically similar to the apoptotic human stem cell-derived donor cell vaccine; iv) apoptotic donor cells, where the cells are made apoptotic by UV irradiation, gamma-irradiation, or other methods not involving incubation in the presence of ECDI. In some cases, tolerizing vaccine cells can be adminstered, e.g., infused (in some cases repeatedly infused) to a subject in need thereof. Tolerizing vaccines can be produced by disrupting (e.g., reducing expression) one or more genes from a cell. For example, genetically modified cells as described throughout the application can be used to make a tolerizing vaccine. For example, cells can have one or more genes that can be disrupted (e.g., reduced expression) including glycoprotein galactosyltransferase alpha 1, 3 (GGTA1), putative cytidine monophosphate-N-acetylneuraminic acid hydroxylase-like protein (CMAH), B4GALNT2, and/or any combination thereof. For example, a cell can have disrupted GGTA1 only, or disrupted CMAH only, or disrupted B4GALNT2 only. A cell can also have disrupted GGTA1 and CMAH, disrupted GGTA1 and B4GALNT2, or disrupted CMAH and B4GALNT2. A cell can have disrupted GGTA1, CMAH, and B4GALNT2. In some cases, the disrupted gene does not include GGTA1. A cell can also express NLRC5 (endogenously or exogenously), while GGTA1 and/or CMAH are disrupted. A cell can also have disrupted C3.

A tolerizing vaccine can be produced with cells comprising additionally expressing one or more transgenes, e.g., as described throughout the application. For example, a tolerizing vaccine can comprise a cell comprising one or more transgenes comprising one or more polynucleotide inserts of Infected cell protein 47 (ICP47), Cluster of differentiation 46 (CD46), Cluster of differentiation 55 (CD55), Cluster of differentiation 59 (CD 59), HLA-E, HLA-G (e.g., HLA-G1, HLA-G2, HLA-G3, HLA-G4, HLA-G5, HLA-G6, or HLA-G7), B2M, PD-L1, PD-L2, CD47, any functional fragments thereof, or any combination thereof. In some cases, a tolerizing vaccine can comprise a genetically modified cell comprising reduced protein expression of GGTA1, CMAH, and B4GALNT2, and transgenes comprising polynucleotides encoding proteins or functional fragments thereof, where the proteins comprise HLA-G1, PD-L1, PD-L2, and CD47. In some cases, a tolerizing vaccine can comprise a genetically modified cell comprising reduced protein expression of GGTA1, CMAH, and B4GALNT2, and transgenes comprising polynucleotides encoding proteins or functional fragments thereof, where the proteins comprise HLA-E, PD-L1, PD-L2, and CD47. In some cases, a tolerizing vaccine can comprise a cell coated with CD47 on its surface. Coating of CD47 on the surface of a cell can be accomplished by biotinylating the cell surface followed by incubating these biotinylated cells with a streptavidin-CD47 chimeric protein. For example, a tolerizing vaccine can comprise a cell coated with CD47 on its surface, where the cell comprises reduced protein expression of GGTA1, CMAH, and B4GALNT2, and transgenes comprising polynucleotides encoding proteins or functional fragments thereof, where the proteins comprise HLA-G1, PD-L1, and PD-L2. A CD47-coated cell can be a non-apoptotic cell. Alternative, a CD47 coated cell can be an apoptotic cell.

In some cases, tolerization may comprise administration of a genetically modified graft. A graft can be a cell, tissue, organ, or a combination. In some cases, immunosuppression is combined with a vaccine or tolerizing graft. In some cases, expression of HLA-G1 on a graft and an MHC or HLA class I deficiency of a graft may have tolerogenic activity independent from administration of a vaccine.

When administered in a subject, a cell of a tolerizing vaccine can have a circulation half-life. A cell of a tolerizing vaccine can have a circulation half-life of at least or at least about 0.1, 0.5, 1, 2, 3, 4, 5, 6, 7, 8, 9, 10, 12, 18, 24, 36, 48, 60, or 72 hours. For example, the circulation half-life of the tolerizing vaccine can be from or from about 0.1 to 0.5; 0.5 to 1.0; 1.0 to 2.0; 1.0 to 3.0; 1.0 to 4.0; 1.0 to 5.0; 5 to 10; 10 to 15; 15 to 24; 24 to 36; 36 to 48; 48 to 60; or 60 to 72 hours. A cell in a tolerizing vaccine can be treated to enhance its circulation half-life. Such treatment can include coating the cell with a protein, e.g., CD47. A cell treated to enhance its circulation half-life can be a non-apoptotic cell. A cell treated to enhance its circulation half-life can be an apoptotic cell. Alternatively, a cell in a tolerizing vaccine can be genetically modified (e.g., insertion of a transgene such as CD47 in its genome) to enhance its circulation half-life. A cell genetically modified to enhance its circulation half-life can be a non-apoptotic cell. A cell genetically modified to enhance its circulation half-life can be an apoptotic cell.

A tolerizing vaccine can have both one or more disrupted genes (e.g., reduced expression) and one or more transgenes. Any genes and/or transgenes as described herein can be used.

A cell that comprises one or more disrupted genes (e.g., reduced expression) can be used as, or be a part of, a tolerizing vaccine. In other words, a cell that comprises one or more disrupted genes can be or can be made into a tolerizing vaccine.

A tolerizing vaccine can have the same genotype and/or phenotype as cells, organs, and/or tissues used in transplantation. Sometimes, the genotype and/or phenotype of a tolerizing vaccine and a transplant are different. A tolerizing vaccine used for a transplant recipient can comprise cells from the transplant graft donor. A tolerizing vaccine used for a transplant recipient can comprise cells that are genetically and/or phenotypically different from the transplant graft. In some cases, a tolerizing vaccine used for a transplant recipient can comprise cells from the transplant graft donor and cells that are genetically and/or phenotypically different from the transplant graft. The cells that are genetically and/or phenotypically different from the transplant graft can be from an animal of the same species of the transplant graft donor.

A source of cells for a tolerizing vaccine can be from a human or non-human animal.

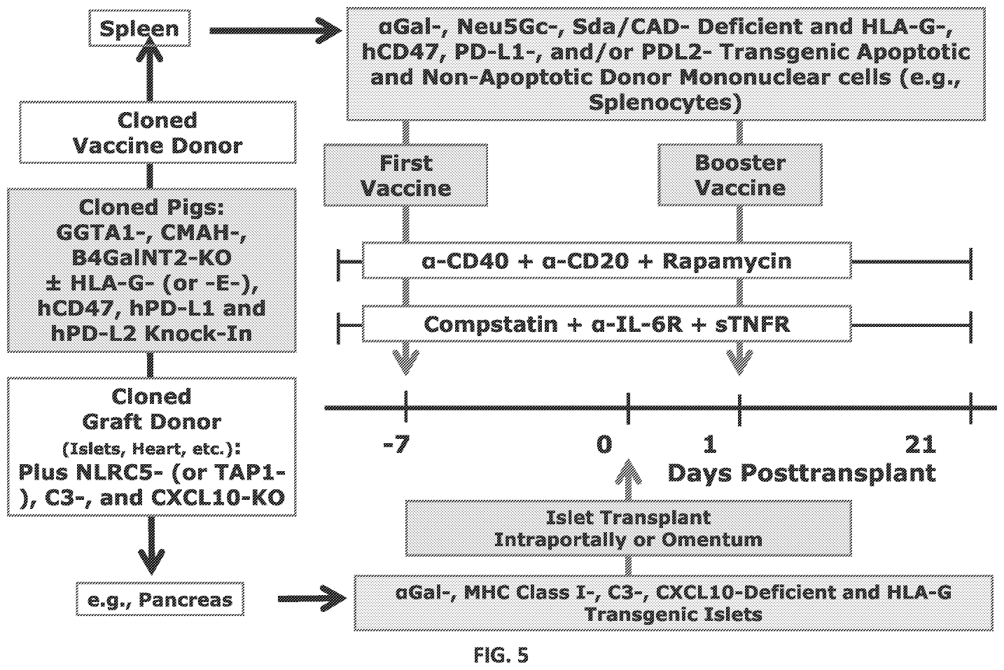

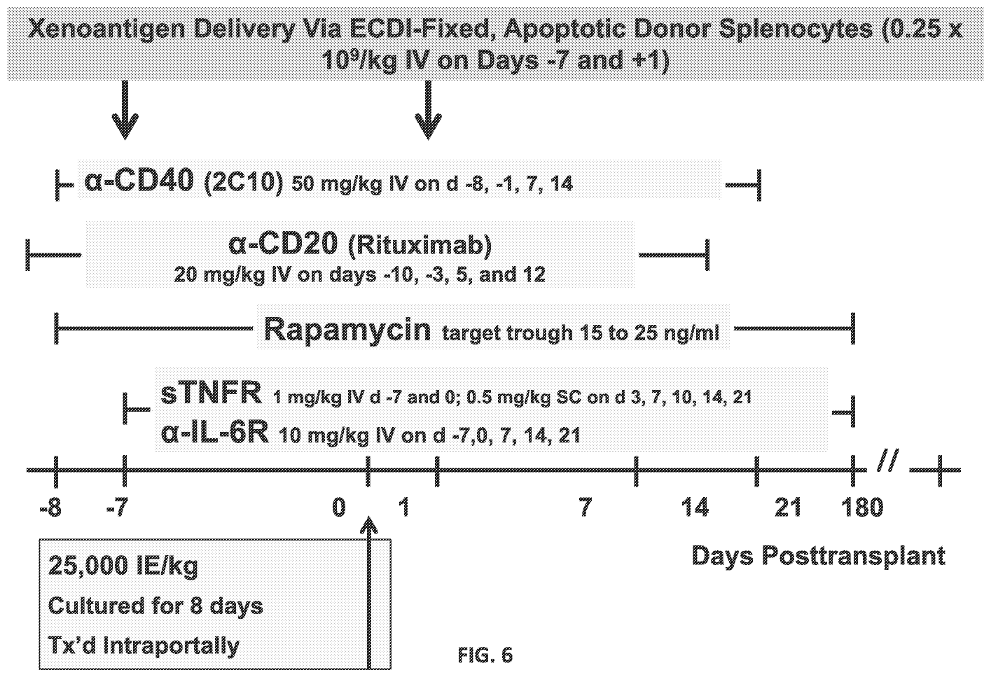

Cells as disclosed throughout the application can be made into a tolerizing vaccine. For example, a tolerizing vaccine can be made of one or more transplanted cells disclosed herein. Alternatively, a tolerizing vaccine can be made of one or more cells that are different from any of the transplanted cells. For example, the cells made into a tolerizing vaccine can be genotypically and/or phenotypically different from any of the transplanted cells. However in some cases, the tolerizing vaccine will express NLRC5 (endogenously or exogenously). A tolerizing vaccine can promote survival of cells, organs, and/or tissues in transplantation. A tolerizing vaccine can be derived from non-human animals that are genotypically identical or similar to donor cells, organs, and/or tissues. For example, a tolerizing vaccine can be cells derived from pigs (e.g., apoptotic pig cells) that are genotypically identical or similar to donor pig cells, organs, and/or tissues. Subsequently, donor cells, organs, and/or tissues can be used in allografts or xenografts. In some cases, cells for a tolerizing vaccine can be from genetically modified animals (e.g., pigs) with reduced expression of GGTA1, CMAH, and B4GalNT2, and having transgenes encoding HLA-G (or HLA-E-), human CD47, human PD-L1 and human PD-L2. Graft donor animals can be generated by further genetically modifying the animals (e.g., pigs) for tolerizing vaccine cells. For example, graft donor animals can be generated by disrupting additional genes (e.g., NLRC5 (or TAP1), C3, and CXCL10) in the abovementioned animals for tolerizing vaccines cells (FIG. 5).

A tolerizing vaccine can comprise non-human animal cells (e.g., non-human mammalian cells). For example, non-human animal cells can be from a pig, a cat, a cow, a deer, a dog, a ferret, a gaur, a goat, a horse, a mouse, a mouflon, a mule, a rabbit, a rat, a sheep, or a primate. Specifically, non-human animal cells can be porcine cells. A tolerizing vaccine can also comprise genetically modified non-human animal cells. For example, genetically modified non-human animal cells can be dead cells (e.g., apoptotic cells). A tolerizing vaccine can also comprise any genetically modified cells disclosed herein.

### Treatment of Cells to Make a Tolerizing Vaccine

A tolerizing vaccine can comprise cells treated with a chemical. In some cases, the treatment can induce apoptosis of the cells. Without being bound by theory, the apoptotic cells can be picked up by host antigen presenting cells (e.g., in the spleen) and presented to host immune cells (e.g., T cells) in a non-immunogenic fashion that leads to induction of anergy in the immune cells (e.g., T cells).

Tolerizing vaccines can comprise apoptotic cells and non-apoptotic cells. An apoptotic cell in a tolerizing vaccine can be genetically identical to a non-apoptotic cell in the tolerizing vaccine. Alternatively, an apoptotic cell in a tolerizing vaccine can be genetically different from a non-apoptotic cell in the tolerizing vaccine. Tolerizing vaccines can comprise fixed cells and non-fixed cells. A fixed cell in a tolerizing vaccine can be genetically identifical to a non-fixed cell in the tolerizing vaccine. Alternatively, a fixed cell in a tolerizing vaccine can be genetically different from a non-fixed cell in the tolerizing vaccine. In some cases, the fixed cell can be a 1-ethyl-3-(3-dimethylaminopropyl)-carbodiimide (ECDI)-fixed cell.

Cells in a tolerizing vaccine can be fixed using a chemical, e.g., ECDI. The fixation can make the cells apoptotic. A tolerizing vaccine, cells, kits and methods disclosed herein can comprise ECDI and/or ECDI treatment. For example, a tolerizing vaccine can be cells, e.g., the genetically modified cell as disclosed herein, that are treated with 1-ethyl-3-(3-dimethylaminopropyl)-carbodiimide (ECDI). In other words, the genetically modified cells as described throughout can be treated with ECDI to create a tolerizing vaccine. A tolerizing vaccine can then be used in transplantation to promote survival of cells, organs, and/or tissues that are transplanted. It is also contemplated that ECDI derivatives, functionalized ECDI, and/or substituted ECDI can also be used to treat the cells for a tolerizing vaccine. In some cases, cells for a tolerizing vaccine can be treated with any suitable carbodiimide derivatives, e.g., ECDI, N, N′-diisopropylcarbodiimide (DIC), N,N′-dicyclohexylcarbodiimide (DCC), and other carbodiimide derivatives understood by those in the art.

Cells for tolerizing vaccines can also be made apoptotic methods not involving incubation in the presence of ECDI, e.g., other chemicals or irradiation such as UV irradiation or gamma-irradiation.

ECDI can chemically cross-link free amine and carboxyl groups, and can effectively induce apoptosis in cells, organs, and/or tissues, e.g., from animal that gave rise to both a tolerizing vaccine and a donor non-human animal. In other words, the same genetically modified animal can give rise to a tolerizing vaccine and cells, tissues and/or organs that are used in transplantation. For example, the genetically modified cells as disclosed herein can be treated with ECDI. This ECDI fixation can lead to the creation of a tolerizing vaccine.

Genetically modified cells that can be used to make a tolerizing vaccine can be derived from: a spleen (including splenic B cells), liver, peripheral blood (including peripheral blood B cells), lymph nodes, thymus, bone marrow, or any combination thereof. For example, cells can be spleen cells, e.g., porcine spleen cells. In some cases, cells can be expanded ex-vivo. In some cases, cells can be derived from fetal, perinatal, neonatal, preweaning, and/or young adult, non-human animals. In some cases, cells can be derived from an embryo of a non-human animal.

Cells in a tolerizing vaccine can also comprise two or more disrupted (e.g., reduced expression) genes, where the two or more disrupted genes can be glycoprotein galactosyltransferase alpha 1,3 (GGTA1), putative cytidine monophosphate-N-acetylneuraminic acid hydroxylase-like protein (CMAH), HLA-E, HLA-G (e.g., HLA-G1, HLA-G2, HLA-G3, HLA-G4, HLA-G5, HLA-G6, or HLA-G7), B2M, and B4GALNT2, any functional fragments thereof, or any combination thereof. In some cases, the two or more disrupted genes do not include GGTA1. As described above, disruption can be a knockout or suppression of gene expression. Knockout can be performed by gene editing, for example, by using a CRISPR/Cas system. Alternatively, suppression of gene expression can be done by knockdown, for example, using RNA interference, shRNA, one or more dominant negative transgenes. In some cases, cells can further comprise one or more transgenes as disclosed herein. For example, one or more transgenes can be CD46, CD55, CD59, or any combination thereof.

Cells in a tolerizing vaccine can also be derived from one or more donor non-human animals. In some cases, cells can be derived from the same donor non-human animal. Cells can be derived from one or more recipient non-human animals. In some cases, cells can be derived from two or more non-human animals (e.g., pig).

A tolerizing vaccine can comprise from or from about 0.001 and about 5.0, e.g., from or from about 0.001 and 1.0, endotoxin unit per kg bodyweight of a prospective recipient. For example, a tolerizing vaccine can comprise from or from about 0.01 to 5.0; 0.01 to 4.5; 0.01 to 4.0, 0.01 to 3.5; 0.01 to 3.0; 0.01 to 2.5; 0.01 to 2.0; 0.01 to 1.5; 0.01 to 1.0; 0.01 to 0.9; 0.01 to 0.8; 0.01 to 0.7; 0.01 to 0.6; 0.01 to 0.5; 0.01 to 0.4; 0.01 to 0.3; 0.01 to 0.2; or 0.01 to 0.1 endotoxin unit per kg bodyweight of a prospective recipient.

A tolerizing vaccine can comprise from or from about 1 to 100 aggregates, per μl. For example, a tolerizing vaccine can comprise from or from about 1 to 5; 1 to 10, or 1 to 20 aggregate per μl. A tolerizing vaccine can comprise at least or at least about 1, 5, 10, 20, 50, or 100 aggregates.

A tolerizing vaccine can trigger a release from or from about 0.001 pg/ml to 10.0 pg/ml, e.g., from or from about 0.001 pg/ml to 1.0 pg/ml, IL-1 beta when about 50,000 frozen to thawed human peripheral blood mononuclear cells are incubated with about 160,000 cells of the tolerizing vaccine (e.g., pig cells). For example, a tolerizing vaccine triggers a release of from or from about 0.001 to 10.0; 0.001 to 5.0; 0.001 to 1.0; 0.001 to 0.8; 0.001 to 0.2; or 0.001 to 0.1 pg/ml IL-1 beta when about 50,000 frozen to thawed human peripheral blood mononuclear cells are incubated with about 160,000 cell of the tolerizing vaccine (e.g., pig cells). A tolerizing vaccine can trigger a release of from or from about 0.001 to 2.0 pg/ml, e.g., from or from about 0.001 to 0.2 pg/ml, IL-6 when about 50,000 frozen to thawed human peripheral blood mononuclear cells are incubated with about 160,000 cells of the tolerizing vaccine (e.g., pig cells). For example, a tolerizing vaccine can trigger a release of from or from about 0.001 to 2.0; 0.001 to 1.0; 0.001 to 0.5; or 0.001 to 0.1 pg/ml IL-6 when about 50,000 frozen to thawed human peripheral blood mononuclear cells are incubated with about 160,000 cells of the tolerizing vaccine (e.g., pig cells).

A tolerizing vaccine can comprise more than or more than about 60%, e.g., more than or more than about 85%, Annexin V positive, apoptotic cells after a 4 hour or after about 4 hours post-release incubation at 37° C. For example, a tolerizing vaccine comprises more than 60%, 70%, 80%, 90%, or 99% Annexin V positive, apoptotic cells after about a 4 hour post-release incubation at 37° C.

A tolerizing vaccine can include from or from about 0.01% to 10%, e.g., from or from about 0.01% to 2%, necrotic cells. For example, a tolerizing vaccine includes from or from about 0.01% to 10%; 0.01% to 7.5%, 0.01% to 5%; 0.01% to 2.5%; or 0.01% to 1% necrotic cells.

Administering a tolerizing vaccine comprising ECDI-treated cells, organs, and/or tissues before, during, and/or after administration of donor cells can induce tolerance for cells, organs, and/or tissues in a recipient (e.g., a human or a non-human animal). ECDI-treated cells can be administered by intravenous infusion.

Tolerance induced by infusion of a tolerizing vaccine comprising ECDI-treated splenocytes is likely dependent on synergistic effects between an intact programmed death 1 receptor—programmed death ligand 1 signaling pathway and CD4⁺CD25⁺Foxp3⁺ regulatory T cells.

Cells in a telorizing vaccine can be made into apoptotic cells (e.g., tolerizing vaccines) not only by ECDI fixation, but also through other methods. For example, any of the genetically modified cells as disclosed throughout, e.g., non-human cells animal cells or human cells (including stem cells), can be made apopototic by exposing the genetically modified cells to UV irradiation. The genetically modified cells can also be made apopototic by exposing it to gamma-irradiation. Other methods, not involving ECDI are also comtemplated, for example, by EtOH fixation.

Cells in a tolerizing vaccine, e.g., ECDI-treated cells, antigen-coupled cells, and/or epitope-coupled cells can comprise donor cells (e.g., cells from the donor of transplant grafts). Cells in a tolerizing vaccine, e.g., ECDI-treated cells, antigen-coupled cells, and/or epitope-coupled cells can comprise recipient cells (e.g., cells from the recipient of transplant grafts). Cells in a tolerizing vaccine, e.g., ECDI-treated cells, antigen-coupled cells, and/or epitope-coupled cells can comprise third party (e.g., neither donor nor recipient) cells. In some cases, third party cells are from a non-human animal of the same species as a recipient and/or donor. In other cases, third party cells are from a non-human animal of a different species as a recipient and/or donor.

ECDI-treatment of cells can be performed in the presence of one or more antigens and/or epitopes. ECDI-treated cells can comprise donor, recipient and/or third party cells. Likewise, antigens and/or epitopes can comprise donor, recipient and/or third party antigens and/or epitopes. In some cases, donor cells are coupled to recipient antigens and/or epitopes (e.g., ECDI-induced coupling). For example, soluble donor antigen derived from genetically engineered and genotypically identical donor cells (e.g., porcine cells) is coupled to recipient peripheral blood mononuclear cells with ECDI and the ECDI-coupled cells are administered via intravenous infusion.

In some cases, recipient cells are coupled to donor antigens and/or epitopes (e.g., ECDI-induced coupling). In some cases, recipient cells are coupled to third party antigens and/or epitopes (e.g., ECDI-induced coupling). In some cases, donor cells are coupled to recipient antigens and/or epitopes (e.g., ECDI-induced coupling). In some cases, donor cells are coupled to third party antigens and/or epitopes (e.g., ECDI-induced coupling). In some cases, third party cells are coupled to donor antigens and/or epitopes (e.g., ECDI-induced coupling). In some cases, third party cells are coupled to recipient antigens and/or epitopes (e.g., ECDI-induced coupling). For example, soluble donor antigen derived from genetically engineered and genotypically identical donor cells (e.g., porcine cells) is coupled to polystyrene nanoparticles with ECDI and the ECDI-coupled cells are administered via intravenous infusion.

Tolerogenic potency of any of these tolerizing cell vaccines can be further optimized by coupling to the surface of cells one or more of the following: IFN-g, NF-kB inhibitors (such as curcumin, triptolide, Bay-117085), vitamin D3, siCD40, cobalt protoporphyrin, insulin B9-23, or other immunomodulatory molecules that modify the function of host antigen-presenting cells and host lymphocytes.

These apoptotic cell vaccines can also be complemented by donor cells engineered to display on their surface molecules (such as FasL, PD-L1, galectin-9, CD8alpha) that trigger apoptotic death of donor-reactive cells.

Tolerizing vaccines dislosed herein can increase the duration of survival of a transplant (e.g., a xenograft or an allograft transplant) in a recipient. Tolerizing vaccines disclosed herein can also reduce or eliminate need for immunosupression following transplantation. Xenograft or allograft transplant can be an organ, tissue, cell or cell line. Xenograft transplants and tolerizing vaccines can also be from different species. Alternatively, xenograft transplants and the tolerizing vaccines can be from the same species. For example, a xenograft transplant and a tolerizing vaccine can be from substantially genetically identical individuals (e.g., the same individual).

In some cases a tolerizing vaccine or negative vaccine can produce synergistic effects in a subject administered a tolerizing or negative vaccine. In other cases, a tolerizing or negative vaccine can produce antagonistic effects in a subject administered a tolerizing or negative vaccine.

The ECDI fixed cells can be formulated into a pharmaceutical composition. For example, the ECDI fixed cells can be combined with a pharmaceutically acceptable excipient. An excipient that can be used is saline. An excipient that can be used is phosphate buffered saline (PBS). The pharmaceutical compositions can be then used to treat patients in need of transplantation.

IV. Method of Making Genetically Modified Non-Human Animals

In order to make a genetically modified non-human animal as described above, various techniques can be used. Disclosed herein are a few examples to create genetically modified animals. It is to be understood that the methods disclosed herein are simply examples, and are not meant to limiting in any way.

### Gene Disruption

Gene disruption can be performed by any methods described above, for example, by knockout, knockdown, RNA interference, dominant negative, etc. A detailed description of the methods is disclosed above in the section regarding genetically modified non-human animals.

### CRISPR/Cas System

Methods described herein can take advantage of a CRISPR/Cas system. For example, double-strand breaks (DSBs) can be generated using a CRISPR/Cas system, e.g., a type II CRISPR/Cas system. A Cas enzyme used in the methods disclosed herein can be Cas9, which catalyzes DNA cleavage. Enzymatic action by Cas9 derived from Streptococcus pyogenes or any closely related Cas9 can generate double stranded breaks at target site sequences which hybridize to 20 nucleotides of a guide sequence and that have a protospacer-adjacent motif (PAM) following the 20 nucleotides of the target sequence.

A vector can be operably linked to an enzyme-coding sequence encoding a CRISPR enzyme, such as a Cas protein. Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof. An unmodified CRISPR enzyme can have DNA cleavage activity, such as Cas9. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence. A vector that encodes a CRISPR enzyme that is mutated to with respect, to a corresponding wild-type enzyme such that the mutated CRISPR enzyme lacks the ability to cleave one or both strands of a target polynucleotide containing a target sequence can be used.

Cas9 can refer to a polypeptide with at least or at least about 50%, 60%, 70%, 80%, 90%, 91%, 92%, 93%, 94%, 95%, 96%, 97%, 98%, 99%, or 100% sequence identity and/or sequence homology to a wild type exemplary Cas9 polypeptide (e.g., Cas9 from S. pyogenes). Cas9 can refer to a polypeptide with at most or at most about 50%, 60%, 70%, 80%, 90%, 91%, 92%, 93%, 94%, 95%, 96%, 97%, 98%, 99%, or 100% sequence identity and/or sequence homology to a wild type exemplary Cas9 polypeptide (e.g., from S. pyogenes). Cas9 can refer to the wild type or a modified form of the Cas9 protein that can comprise an amino acid change such as a deletion, insertion, substitution, variant, mutation, fusion, chimera, or any combination thereof.

S. pyogenes Cas9 (SpCas9) can be used as a CRISPR endonuclease for genome engineering. However, others can be used. In some cases, a different endonuclease may be used to target certain genomic targets. In some cases, synthetic SpCas9-derived variants with non-NGG PAM sequences may be used. Additionally, other Cas9 orthologues from various species have been identified and these “non-SpCas9s” can bind a variety of PAM sequences that could also be useful for the present invention. For example, the relatively large size of SpCas9 (approximately 4 kb coding sequence) can lead to plasmids carrying the SpCas9 cDNA that may not be efficiently expressed in a cell. Conversely, the coding sequence for Staphylococcus aureus Cas9 (SaCas9) is approximatelyl kilo base shorter than SpCas9, possibly allowing it to be efficiently expressed in a cell. Similar to SpCas9, the SaCas9 endonuclease is capable of modifying target genes in mammalian cells in vitro and in mice in vivo. In some cases, a Cas protein may target a different PAM sequence. In some cases, a target gene, such as NLRC5, may be adjacent to a Cas9 PAM, 5′-NGG, for example. In other cases, other Cas9 orthologs may have different PAM requirements. For example, other PAMs such as those of S. thermophilus (5′-NNAGAA for CRISPR1 and 5′-NGGNG for CRISPR3) and Neisseria meningiditis (5′-NNNNGATT) may also be found adjacent to a target gene, such as NLRC5. A transgene of the present invention may be inserted adjacent to any PAM sequence from any Cas, or Cas derivative, protein. In some cases, a PAM can be found every, or about every, 8 to 12 base pairs in a genome. A PAM can be found every 1 to 15 basepairs in a genome. A PAM can also be found every 5 to 20 basepairs in a genome. In some cases, a PAM can be found every 5, 6, 7, 8, 9, 10, 11, 12, 13, 14, 15, 16, 17, 18, 19, 20, or more basepairs in a genome. A PAM can be found at or between every 5-100 base pairs in a genome.

For example, for a S. pyogenes system, a target gene sequence can precede (i.e., be 5′ to) a 5′-NGG PAM, and a 20-nt guide RNA sequence can base pair with an opposite strand to mediate a Cas9 cleavage adjacent to a PAM. In some cases, an adjacent cut may be or may be about 3 base pairs upstream of a PAM. In some cases, an adjacent cut may be or may be about 10 base pairs upstream of a PAM. In some cases, an adjacent cut may be or may be about 0-20 base pairs upstream of a PAM. For example, an adjacent cut can be next to, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 base pairs upstream of a PAM. An adjacent cut can also be downstream of a PAM by 1 to 30 base pairs.

Alternatives to S. pyogenes Cas9 may include RNA-guided endonucleases from the Cpf1 family that display cleavage activity in mammalian cells. Unlike Cas9 nucleases, the result of Cpf1-mediated DNA cleavage is a double-strand break with a short 3′ overhang. Cpf1's staggered cleavage pattern may open up the possibility of directional gene transfer, analogous to traditional restriction enzyme cloning, which may increase the efficiency of gene editing. Like the Cas9 variants and orthologues described above, Cpf1 may also expand the number of sites that can be targeted by CRISPR to AT-rich regions or AT-rich genomes that lack the NGG PAM sites favored by SpCas9.

A vector that encodes a CRISPR enzyme comprising one or more nuclear localization sequences (NLSs) can be used. For example, there can be or be about 1, 2, 3, 4, 5, 6, 7, 8, 9, 10 NLSs used. A CRISPR enzyme can comprise the NLSs at or near the ammo-terminus, about or more than about 1, 2, 3, 4, 5, 6, 7, 8, 9, 10 NLSs at or near the carboxy-terminus, or any combination of these (e.g., one or more NLS at the ammo-terminus and one or more NLS at the carboxy terminus). When more than one NLS is present, each can be selected independently of others, such that a single NLS can be present in more than one copy and/or in combination with one or more other NLSs present in one or more copies.

CRISPR enzymes used in the methods can comprise at most 6 NLSs. An NLS is considered near the N- or C-terminus when the nearest amino acid to the NLS is within about 50 amino acids along a polypeptide chain from the N- or C-terminus, e.g., within 1, 2, 3, 4, 5, 10, 15, 20, 25, 30, 40, or 50 amino acids.

Guide RNA

As used herein, the term “guide RNA” and its grammatical equivalents can refer to an RNA which can be specific for a target DNA and can form a complex with Cas protein. An RNA/Cas complex can assist in “guiding” Cas protein to a target DNA.

A method disclosed herein also can comprise introducing into a cell or embryo at least one guide RNA or nucleic acid, e.g., DNA encoding at least one guide RNA. A guide RNA can interact with a RNA-guided endonuclease to direct the endonuclease to a specific target site, at which site the 5′ end of the guide RNA base pairs with a specific protospacer sequence in a chromosomal sequence.

A guide RNA can comprise two RNAs, e.g., CRISPR RNA (crRNA) and transactivating crRNA (tracrRNA). A guide RNA can sometimes comprise a single-chain RNA, or single guide RNA (sgRNA) formed by fusion of a portion (e.g., a functional portion) of crRNA and tracrRNA. A guide RNA can also be a dualRNA comprising a crRNA and a tracrRNA. Furthermore, a crRNA can hybridize with a target DNA.

As discussed above, a guide RNA can be an expression product. For example, a DNA that encodes a guide RNA can be a vector comprising a sequence coding for the guide RNA. A guide RNA can be transferred into a cell or organism by transfecting the cell or organism with an isolated guide RNA or plasmid DNA comprising a sequence coding for the guide RNA and a promoter. A guide RNA can also be transferred into a cell or organism in other way, such as using virus-mediated gene delivery.

A guide RNA can be isolated. For example, a guide RNA can be transfected in the form of an isolated RNA into a cell or organism. A guide RNA can be prepared by in vitro transcription using any in vitro transcription system known in the art. A guide RNA can be transferred to a cell in the form of isolated RNA rather than in the form of plasmid comprising encoding sequence for a guide RNA.

A guide RNA can comprise three regions: a first region at the 5′ end that can be complementary to a target site in a chromosomal sequence, a second internal region that can form a stem loop structure, and a third 3′ region that can be single-stranded. A first region of each guide RNA can also be different such that each guide RNA guides a fusion protein to a specific target site. Further, second and third regions of each guide RNA can be identical in all guide RNAs.

A first region of a guide RNA can be complementary to sequence at a target site in a chromosomal sequence such that the first region of the guide RNA can base pair with the target site. In some cases, a first region of a guide RNA can comprise from or from about 10 nucleotides to 25 nucleotides (i.e., from 10 nts to 25 nts; or from about 10 nts to about 25 nts; or from 10 nts to about 25 nts; or from about 10 nts to 25 nts) or more. For example, a region of base pairing between a first region of a guide RNA and a target site in a chromosomal sequence can be or can be about 10, 11, 12, 13, 14, 15, 16, 17, 18, 19, 20, 22, 23, 24, 25, or more nucleotides in length. Sometimes, a first region of a guide RNA can be or can be about 19, 20, or 21 nucleotides in length.

A guide RNA can also comprises a second region that forms a secondary structure. For example, a secondary structure formed by a guide RNA can comprise a stem (or hairpin) and a loop. A length of a loop and a stem can vary. For example, a loop can range from or from about 3 to 10 nucleotides in length, and a stem can range from or from about 6 to 20 base pairs in length. A stem can comprise one or more bulges of 1 to 10 or about 10 nucleotides. The overall length of a second region can range from or from about 16 to 60 nucleotides in length. For example, a loop can be or can be about 4 nucleotides in length and a stem can be or can be about 12 base pairs.

A guide RNA can also comprise a third region at the 3′ end that can be essentially single-stranded. For example, a third region is sometimes not complementarity to any chromosomal sequence in a cell of interest and is sometimes not complementarity to the rest of a guide RNA. Further, the length of a third region can vary. A third region can be more than or more than about 4 nucleotides in length. For example, the length of a third region can range from or from about 5 to 60 nucleotides in length.

A guide RNA can target any exon or intron of a gene target. In some cases, a guide can target exon 1 or 2 of a gene, in other cases; a guide can target exon 3 or 4 of a gene. A composition can comprise multiple guide RNAs that all target the same exon or in some cases, multiple guide RNAs that can target different exons. An exon and an intron of a gene can be targeted.

A guide RNA can target a nucleic acid sequence of or of about 20 nucleotides. A target nucleic acid can be less than or less than about 20 nucleotides. A target nucleic acid can be at least or at least about 5, 10, 15, 16, 17, 18, 19, 20, 21, 22, 23, 24, 25, 30, or anywhere between 1-100 nucleotides in length. A target nucleic acid can be at most or at most about 5, 10, 15, 16, 17, 18, 19, 20, 21, 22, 23, 24, 25, 30, 40, 50, or anywhere between 1-100 nucleotides in length. A target nucleic acid sequence can be or can be about 20 bases immediately 5′ of the first nucleotide of the PAM. A guide RNA can target a nucleic acid sequence. A target nucleic acid can be at least or at least about 1-10, 1-20, 1-30, 1-40, 1-50, 1-60, 1-70, 1-80, 1-90, or 1-100.

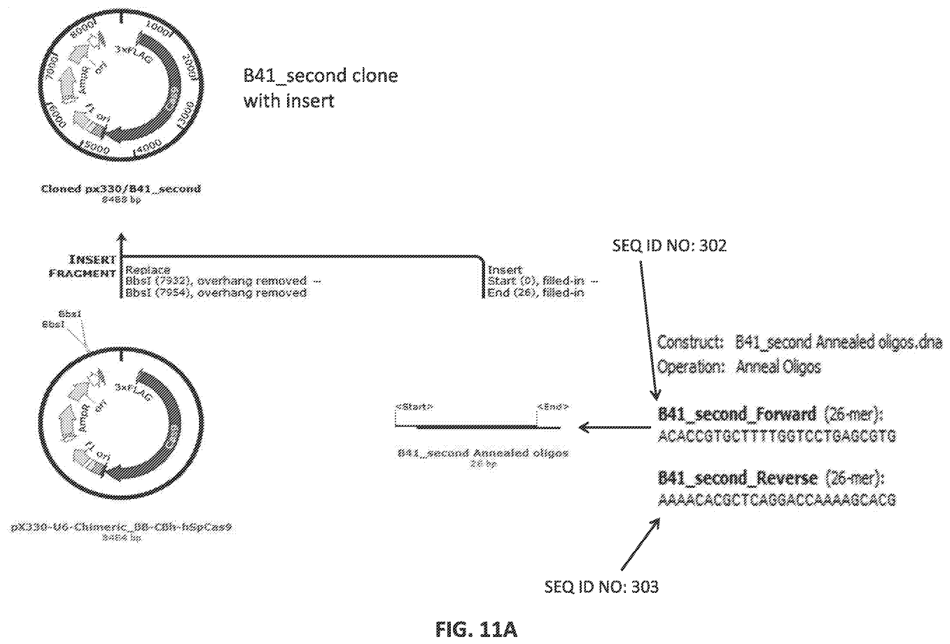

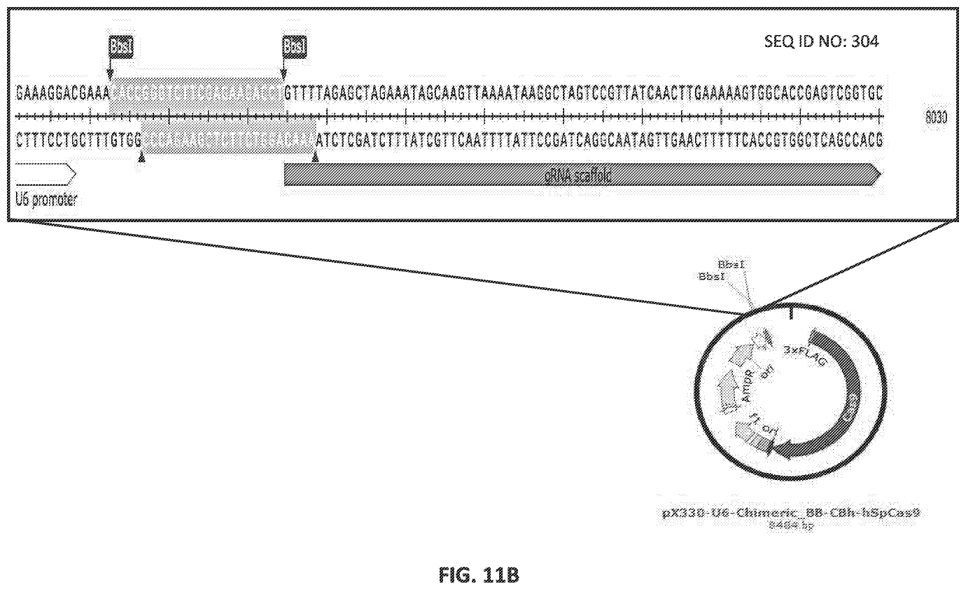

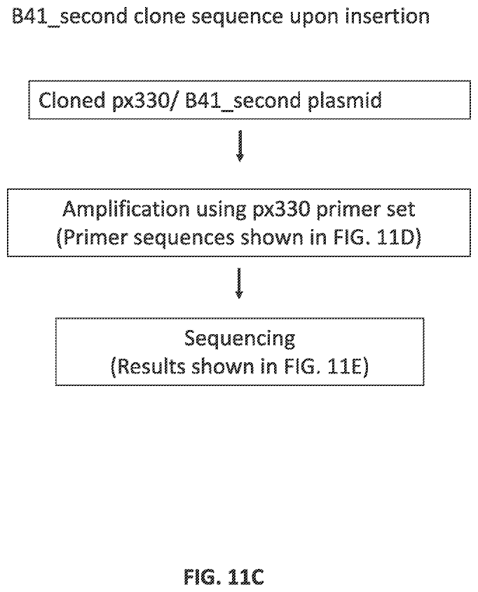

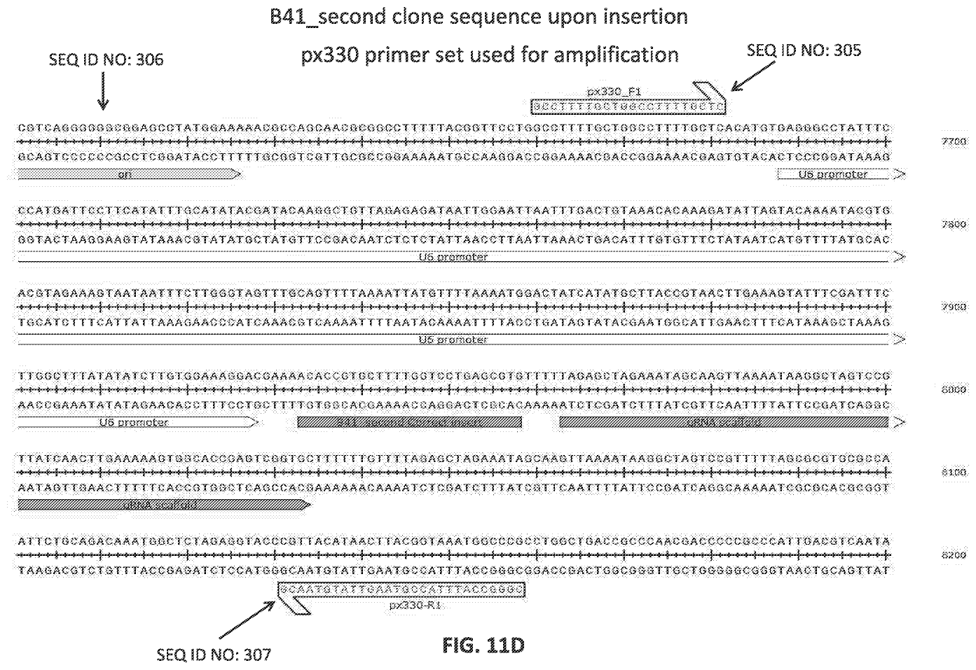

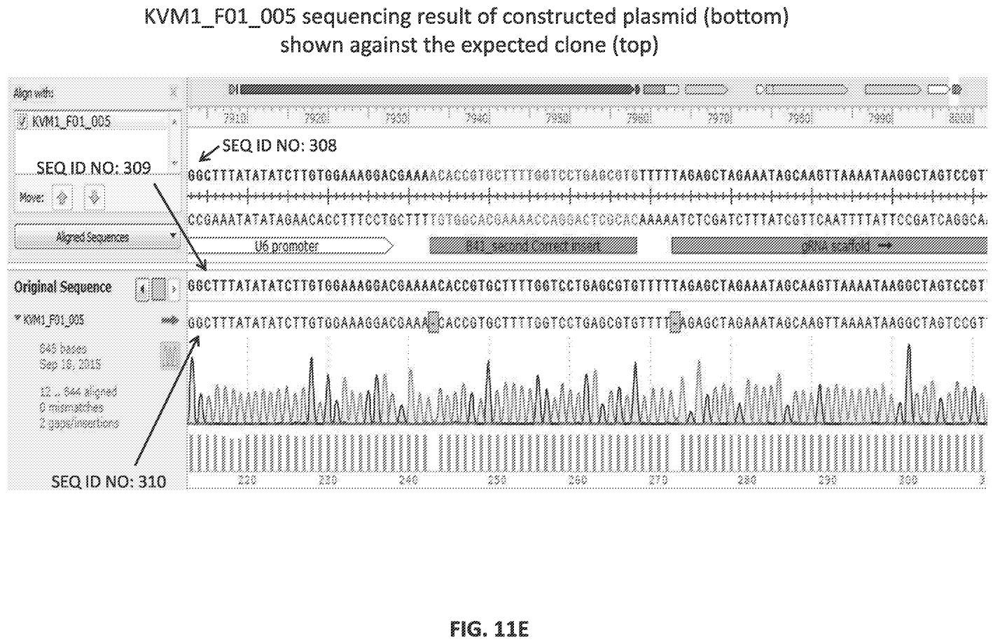

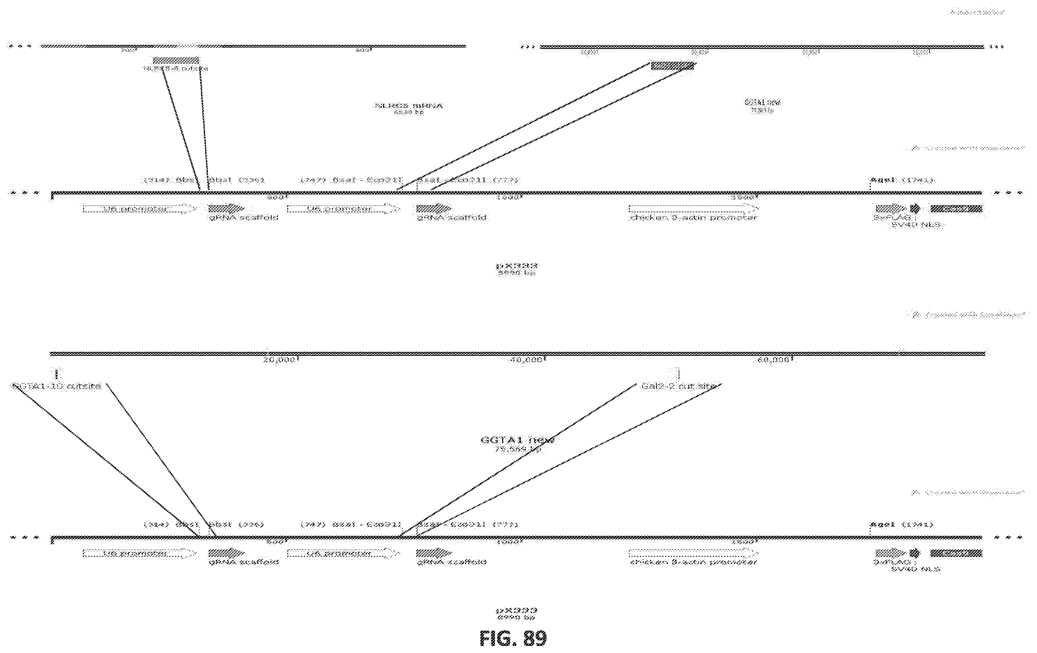

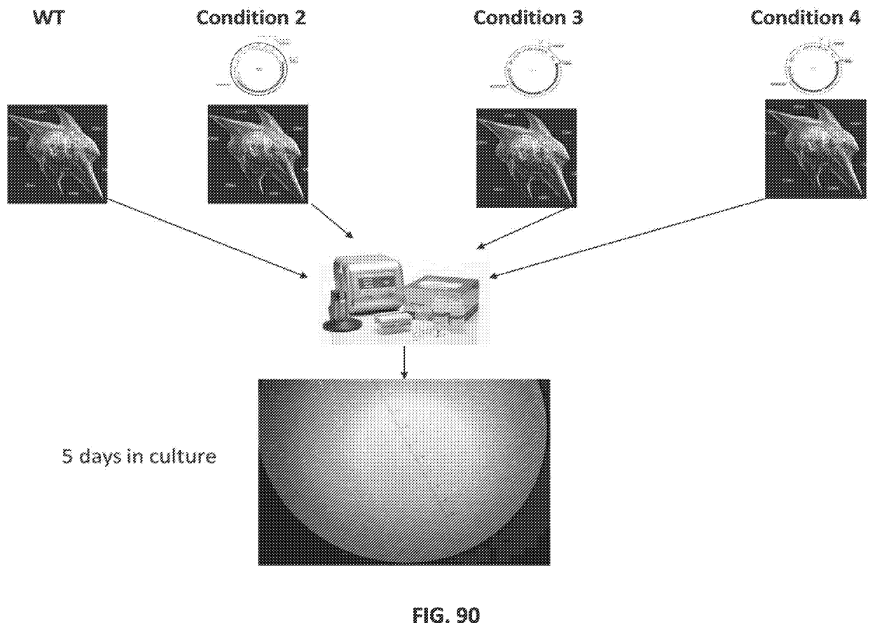

A guide nucleic acid, for example, a guide RNA, can refer to a nucleic acid that can hybridize to another nucleic acid, for example, the target nucleic acid or protospacer in a genome of a cell. A guide nucleic acid can be RNA. A guide nucleic acid can be DNA. The guide nucleic acid can be programmed or designed to bind to a sequence of nucleic acid site-specifically. A guide nucleic acid can comprise a polynucleotide chain and can be called a single guide nucleic acid. A guide nucleic acid can comprise two polynucleotide chains and can be called a double guide nucleic acid. A guide RNA can be introduced into a cell or embryo as an RNA molecule. For example, a RNA molecule can be transcribed in vitro and/or can be chemically synthesized. An RNA can be transcribed from a synthetic DNA molecule, e.g., a gBlocks® gene fragment. A guide RNA can then be introduced into a cell or embryo as an RNA molecule. A guide RNA can also be introduced into a cell or embryo in the form of a non-RNA nucleic acid molecule, e.g., DNA molecule. For example, a DNA encoding a guide RNA can be operably linked to promoter control sequence for expression of the guide RNA in a cell or embryo of interest. A RNA coding sequence can be operably linked to a promoter sequence that is recognized by RNA polymerase III (Pol III). Plasmid vectors that can be used to express guide RNA include, but are not limited to, px330 vectors and px333 vectors (FIG. 11 and FIG. 89). In some cases, a plasmid vector (e.g., px333 vector) can comprise at least two guide RNA-encoding DNA sequences. A px333 vector can be used, for example, to introduce GGTA1-10 and Gal2-2, or GGTA1-10, Gal2-2, and NLRC5-6. In other cases, NLRC5-6 and Gal2-2 can be introduced with a px333 vector.

A DNA sequence encoding a guide RNA can also be part of a vector. Further, a vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. A DNA molecule encoding a guide RNA can also be linear. A DNA molecule encoding a guide RNA can also be circular.

When DNA sequences encoding an RNA-guided endonuclease and a guide RNA are introduced into a cell, each DNA sequence can be part of a separate molecule (e.g., one vector containing an RNA-guided endonuclease coding sequence and a second vector containing a guide RNA coding sequence) or both can be part of a same molecule (e.g., one vector containing coding (and regulatory) sequence for both an RNA-guided endonuclease and a guide RNA).

Guide RNA can target a gene in a pig or a pig cell. In some cases, guide RNA can target a pig NLRC5 gene, e.g., sequences listed in Table 4. In some cases, guide RNA can be designed to target pig NLRC5, GGTA1 or CMAH gene. Exemplary oligonucleotides for making the guide RNA are listed in Table 5. In some cases, at least two guide RNAs are introduced. At least two guide RNAs can each target two genes. For example, in some cases, a first guide RNA can target GGTA1 and a second guide RNA can target Gal2-2. In some cases, a first guide RNA can target NLRC5 and a second guide RNA can target Gal2-2. In other cases, a first guide RNA can target GGTA1-10 and a second guide RNA can target Gal2-2.

A guide nucleic acid can comprise one or more modifications to provide a nucleic acid with a new or enhanced feature. A guide nucleic acid can comprise a nucleic acid affinity tag. A guide nucleic acid can comprise synthetic nucleotide, synthetic nucleotide analog, nucleotide derivatives, and/or modified nucleotides.

In some cases, a gRNA can comprise modifications. A modification can be made at any location of a gRNA. More than one modification can be made to a single gRNA. A gRNA can undergo quality control after a modification. In some cases, quality control may include PAGE, HPLC, MS, or any combination thereof.

A modification of a gRNA can be a substitution, insertion, deletion, chemical modification, physical modification, stabilization, purification, or any combination thereof.

A gRNA can also be modified by 5′adenylate, 5′ guanosine-triphosphate cap, 5′N⁷-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencer 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-0-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, or any combination thereof.

In some cases, a modification is permanent. In other cases, a modification is transient. In some cases, multiple modifications are made to a gRNA. A gRNA modification may alter physio-chemical properties of a nucleotide, such as their conformation, polarity, hydrophobicity, chemical reactivity, base-pairing interactions, or any combination thereof.

A modification can also be a phosphorothioate substitute. In some cases, a natural phosphodiester bond may be susceptible to rapid degradation by cellular nucleases and; a modification of internucleotide linkage using phosphorothioate (PS) bond substitutes can be more stable towards hydrolysis by cellular degradation. A modification can increase stability in a gRNA. A modification can also enhance biological activity. In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

TABLE 4
Exemplary Sequences of the NLRC5 gene to be
targeted by guide RNAs

| SEQ ID | |
|---|---|
| No. | Sequence (5′-3′) |
| 61 | ggggaggaagaacttcacct |
| 62 | gtaggacgaccctctgtgtg |
| 63 | gaccctctgtgtggggtctg |
| 64 | ggctcggttccattgcaaga |
| 65 | gctcggttccattgcaagat |
| 66 | ggttccattgcaagatgggc |
| 67 | gtcccctcctgagtgtcgaa |
| 68 | gcctcaggtacagatcaaaa |
| 69 | ggacctgggtgccaggaacg |
| 70 | gtacccagagtcagatcacc |
| 71 | gtacccagagtcagatcacc |
| 72 | gtgcccttcgacactcagga |
| 73 | gtgcccttcgacactcagga |
| 74 | gtgcccttcgacactcagga |
| 75 | gggggccccaaggcagaaga |
| 76 | ggcagtcttccagtacctgg |

TABLE 5
Exemplary oligonucleotides for making guide RNA constructs

| | | | SEQ | |
|---|---|---|---|---|
| | SEQ | | ID | |
| Gene | ID No. | Forward sequence (5′ to 3′) | No. | Reverse sequence (5′ to 3′) |
| NLRC5 | 77 | acaccggggaggaagaacttcacctg | 78 | aaaacaggtgaagttcttcctccccg |
| NLRC5 | 79 | acaccgtaggacgaccctctgtgtgg | 80 | aaaaccacacagagggtcgtcctacg |
| NLRC5 | 81 | acaccgaccctctgtgtggggtctgg | 82 | aaaaccagaccccacacagagggtcg |
| NLRC5 | 83 | acaccggctcggttccattgcaagag | 84 | aaaactcttgcaatggaaccgagccg |
| NLRC5 | 85 | acaccgctcggttccattgcaagatg | 86 | aaaacatcttgcaatggaaccgagcg |
| NLRC5 | 87 | acaccggttccattgcaagatgggcg | 88 | aaaacgcccatcttgcaatggaaccg |
| NLRC5 | 89 | acaccgtcccctcctgagtgtcgaag | 90 | aaaacttcgacactcaggaggggacg |
| NLRC5 | 91 | acaccgcctcaggtacagatcaaaag | 92 | aaaacttttgatctgtacctgaggcg |
| NLRC5 | 93 | acaccggacctgggtgccaggaacgg | 94 | aaaaccgttcctggcacccaggtccg |
| NLRC5 | 95 | acaccgtacccagagtcagatcaccg | 96 | aaaacggtgatctgactctgggtacg |
| NLRC5 | 97 | acaccgtacccagagtcagatcaccg | 98 | aaaacggtgatctgactctgggtacg |
| NLRC5 | 99 | acaccgtgcccttcgacactcaggag | 100 | aaaactcctgagtgtcgaagggcacg |
| NLRC5 | 101 | acaccgtgcccttcgacactcaggag | 102 | aaaactcctgagtgtcgaagggcacg |
| NLRC5 | 103 | acaccgtgcccttcgacactcaggag | 104 | aaaactcctgagtgtcgaagggcacg |
| NLRC5 | 105 | acaccgggggccccaaggcagaagag | 106 | aaaactcttctgccttggggcccccg |
| NLRC5 | 107 | acaccggcagtcttccagtacctggg | 108 | aaaacccaggtactggaagactgccg |
| GGTA1 | 109 | caccgagaaaataatgaatgtcaa | 110 | aaacttgacattcattattactc |
| CMAH | 111 | caccgagtaaggtacgtgatctgt | 112 | aaacacagatcacgtaccttactc |

Homologous Recombination

Homologous recombination can also be used for any of the relevant genetic modifications as disclosed herein. Homologous recombination can permit site-specific modifications in endogenous genes and thus novel modifications can be engineered into a genome. For example, the ability of homologous recombination (gene conversion and classical strand breakage/rejoining) to transfer genetic sequence information between DNA molecules can render targeted homologous recombination and can be a powerful method in genetic engineering and gene manipulation.

Cells that have undergone homologous recombination can be identified by a number of methods. For example, a selection method can detect an absence of an immune response against a cell, for example by a human anti-gal antibody. A selection method can also include assessing a level of clotting in human blood when exposed to a cell or tissue. Selection via antibiotic resistance can be used for screening.

### Making Transgenic Non-Human Animals

Random Insertion

One or more transgenes of the methods described herein can be inserted randomly to any locus in a genome of a cell. These transgenes can be functional if inserted anywhere in a genome. For instance, a transgene can encode its own promoter or can be inserted into a position where it is under the control of an endogenous promoter. Alternatively, a transgene can be inserted into a gene, such as an intron of a gene or an exon of a gene, a promoter, or a non-coding region. A transgene can be integrated into a first exon of a gene.

A DNA encoding a transgene sequences can be randomly inserted into a chromosome of a cell. A random integration can result from any method of introducing DNA into a cell known to one of skill in the art. This can include, but is not limited to, electroporation, sonoporation, use of a gene gun, lipotransfection, calcium phosphate transfection, use of dendrimers, microinjection, use of viral vectors including adenoviral, AAV, and retroviral vectors, and/or group II ribozymes.

A DNA encoding a transgene can also be designed to include a reporter gene so that the presence of the transgene or its expression product can be detected via activation of the reporter gene. Any reporter gene known in the art can be used, such as those disclosed above. By selecting in cell culture those cells in which a reporter gene has been activated, cells can be selected that contain a transgene.

A DNA encoding a transgene can be introduced into a cell via electroporation (FIG. 90). A DNA can also be introduced into a cell via lipofection, infection, or transformation. Electroporation and/or lipofection can be used to transfect fibroblast cells.

Expression of a transgene can be verified by an expression assay, for example, qPCR or by measuring levels of RNA. Expression level can be indicative also of copy number. For example, if expression levels are extremely high, this can indicate that more than one copy of a transgene was integrated in a genome. Alternatively, high expression can indicate that a transgene was integrated in a highly transcribed area, for example, near a highly expressed promoter. Expression can also be verified by measuring protein levels, such as through Western blotting.

Site Specific Insertion

Inserting one or more transgenes in any of the methods disclosed herein can be site-specific. For example, one or more transgenes can be inserted adjacent to a promoter, for example, adjacent to or near a Rosa26 promoter.

Modification of a targeted locus of a cell can be produced by introducing DNA into cells, where the DNA has homology to the target locus. DNA can include a marker gene, allowing for selection of cells comprising the integrated construct. Homologous DNA in a target vector can recombine with a chromosomal DNA at a target locus. A marker gene can be flanked on both sides by homologous DNA sequences, a 3′ recombination arm, and a 5′ recombination arm.

A variety of enzymes can catalyze insertion of foreign DNA into a host genome. For example, site-specific recombinases can be clustered into two protein families with distinct biochemical properties, namely tyrosine recombinases (in which DNA is covalently attached to a tyrosine residue) and serine recombinases (where covalent attachment occurs at a serine residue). In some cases, recombinases can comprise Cre, fC31 integrase (a serine recombinase derived from Streptomyces phage fC31), or bacteriophage derived site-specific recombinases (including Flp, lambda integrase, bacteriophage HK022 recombinase, bacteriophage R4 integrase and phage TP901-1 integrase).

Expression control sequences can also be used in constructs. For example, an expression control sequence can comprise a constitutive promoter, which is expressed in a wide variety of cell types. For example, among suitable strong constitutive promoters and/or enhancers are expression control sequences from DNA viruses (e.g., SV40, polyoma virus, adenoviruses, adeno-associated virus, pox viruses, CMV, HSV, etc.) or from retroviral LTRs. Tissue-specific promoters can also be used and can be used to direct expression to specific cell lineages. While experiments discussed in the Examples below will be conducted using a Rosa26 gene promoter, other Rosa26-related promoters capable of directing gene expression can be used to yield similar results, as will be evident to those of skill in the art. Therefore, the description herein is not meant to be limiting, but rather disclose one of many possible examples. In some cases, a shorter Rosa26 5′-upstream sequences, which can nevertheless achieve the same degree of expression, can be used. Also useful are minor DNA sequence variants of a Rosa26 promoter, such as point mutations, partial deletions or chemical modifications.

A Rosa26 promoter is expressible in mammals. For example, sequences that are similar to the 5′ flanking sequence of a pig Rosa26 gene, including, but not limited to, promoters of Rosa26 homologues of other species (such as human, cattle, mouse, sheep, goat, rabbit and rat), can also be used. A Rosa26 gene can be sufficiently conserved among different mammalian species and other mammalian Rosa26 promoters can also be used.

The CRISPR/Cas system can be used to perform site specific insertion. For example, a nick on an insertion site in the genome can be made by CRISPR/Cas to facilitate the insertion of a transgene at the insertion site.

The methods described herein, can utilize techniques which can be used to allow a DNA or RNA construct entry into a host cell include, but are not limited to, calcium phosphate/DNA coprecipitation, microinjection of DNA into a nucleus, electroporation, bacterial protoplast fusion with intact cells, transfection, lipofection, infection, particle bombardment, sperm mediated gene transfer, or any other technique known by one skilled in the art.

Certain aspects disclosed herein can utilize vectors. Any plasmids and vectors can be used as long as they are replicable and viable in a selected host. Vectors known in the art and those commercially available (and variants or derivatives thereof) can be engineered to include one or more recombination sites for use in the methods. Vectors that can be used include, but not limited to eukaryotic expression vectors such as pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMClneo, and pOG44 (Stratagene, Inc.), and pYES2, pAC360, pBlueBa-cHis A, B, and C, pVL1392, pBlueBac111, pCDM8, pcDNA1, pZeoSV, pcDNA3, pREP4, pCEP4, and pEBVHis (Invitrogen, Corp.), and variants or derivatives thereof.

These vectors can be used to express a gene, e.g., a transgene, or portion of a gene of interest. A gene of portion or a gene can be inserted by using known methods, such as restriction enzyme-based techniques.

Making a Similar Genetically Modified Non-Human Animal Using Cell Nuclear Transfer

An alternative method of making a genetically modified non-human animal can be by cell nuclear transfer. A method of making genetically modified non-human animals can comprise a) producing a cell with reduced expression of one or more genes and/or comprise exogenous polynucleotides disclosed herein; b) providing a second cell and transferring a nucleus of the resulting cell from a) to the second cell to generate an embryo generating an embryo; c) growing the embryo into the genetically modified non-human animal. A cell in this method can be an enucleated cell. The cell of a) can be made using any methods, e.g., gene disruption and/or insertion described herein or known in the art.

This method can be used to make a similar genetically modified non-human animal disclosed herein. For example, a method of making a genetically modified non-human animal can comprise: a) producing a cell with reduced expression of NLRC5, TAP1 and/or C3; b) providing a second cell and transferring a nucleus of the resulting cell from a) to the second cell to generate an embryo; and c) growing the embryo to the genetically modified non-human animal. A cell in this method can be an enucleated cell.

Cells used in this method can be from any disclosed genetically modified cells as described herein. For example, disrupted genes are not limited to NRLC5, TAP1, and/or C3. Other combinations of gene disruptions and transgenes can be found throughout disclosure herein. For example, a method can comprise providing a first cell from any non-human animal disclosed herein; providing a second cell; transferring a nucleus of the first cell of a) to the second cell of b); generating an embryo from the product of c); and growing the embryo to the genetically modified non-human animal.

A cell of a) in the methods disclosed herein can be a zygote. The zygote can be formed by joining: i) of a sperm of a wild-type non-human animal and an ovum of a wild-type non-human animal; ii) a sperm of a wild-type non-human animal and an ovum of a genetically modified non-human animal; iii) a sperm of a genetically modified non-human animal and an ovum of a wild-type non-human animal; and/or iv) a sperm of a genetically modified non-human animal and an ovum of a genetically modified non-human animal. A non-human animal can be a pig.

One or more genes in a cell of a) in the methods disclosed herein can be disrupted by generating breaks at desired locations in the genome. For example, breaks can be double-stranded breaks (DSBs). DSBs can be generated using a nuclease comprising Cas (e.g., Cas9), ZFN, TALEN, and maganuclease. Nuclease can be a naturally-existing or a modified nuclease. A nucleic acid encoding a nuclease can be delivered to a cell, where the nuclease is expressed. Cas9 and guide RNA targeting a gene in a cell can be delivered to the cell. In some cases, mRNA molecules encoding Cas9 and guide RNA can be injected into a cell. In some cases, a plasmid encoding Cas9 and a different plasmid encoding guide RNA can be delivered into a cell (e.g., by infection). In some cases, a plasmid encoding both Cas9 and guide RNA can be delivered into a cell (e.g., by infection).

As described above, following DSBs, one or more genes can be disrupted by DNA repairing mechanisms, such as homologous recombination (HR) and/or nonhomologous end-joining (NHEJ). A method can comprise inserting one or more transgenes to a genome of the cell of a). One or more transgenes can comprise ICP47, CD46, CD55, CD59, HLA-E, HLA-G (e.g., HLA-G1, HLA-G2, HLA-G3, HLA-G4, HLA-G5, HLA-G6, or HLA-G7), B2M, any functional fragments thereof, and/or any combination thereof.

The methods provided herein can comprise inserting one or more transgenes where the one or more transgenes can be any transgene in any non-human animal or genetically modified cell disclosed herein.

Also disclosed herein are methods of making a non-human animal using a cell from a genetically modified non-human animal. A cell can be from any genetically modified non-human animal disclosed herein. A method can comprise: a) providing a cell from a genetically identified non-human animal; b) providing a cell; c) transferring a nucleus of the cell of a) to the cell of b); c) generating an embryo from the product of c); and d) growing the embryo to the genetically modified non-human animal. A cell of this method can be an enucleated cell.

Further, cells of a) in the methods can be any cell from a genetically modified non-human animal. For example, a cell of a) in methods disclosed herein can be a somatic cell, such as a fibroblast cell or a fetal fibroblast cell.

An enucleated cell in the methods can be any cell from an organism. For example, an enucleated cell is a porcine cell. An enucleated cell can be an ovum, for example, an enucleated unfertilized ovum.

Genetically modified non-human animal disclosed herein can be made using any suitable techniques known in the art. For example, these techniques include, but are not limited to, microinjection (e.g., of pronuclei), sperm-mediated gene transfer, electroporation of ova or zygotes, and/or nuclear transplantation.

A method of making similar genetically modified non-human animals can comprise a) disrupting one or more genes in a cell, b) generating an embryo using the resulting cell of a); and c) growing the embryo into the genetically modified non-human animal.

A cell of a) in the methods disclosed herein can be a somatic cell. There is no limitation on a type or source of a somatic cell. For example, it can be from a pig or from cultured cell lines or any other viable cell. A cell can also be a dermal cell, a nerve cell, a cumulus cell, an oviduct epithelial cell, a fibroblast cell (e.g., a fetal fibroblast cell), or hepatocyte. A cell of a) in the methods disclosed herein can be from a wild-type non-human animal, a genetically modified non-human animal, or a genetically modified cell. Furthermore, a cell of b) can be an enucleated ovum (e.g., an enucleated unfertilized ovum).

Enucleation can also be performed by known methods. For example, metaphase II oocytes can be placed in either HECM, optionally containing or containing about 7-10 micrograms per milliliter cytochalasin B, for immediate enucleation, or can be placed in a suitable medium (e.g., an embryo culture medium such as CRlaa, plus 10% estrus cow serum), and then enucleated later (e.g., not more than 24 hours later or 16-18 hours later). Enucleation can also be accomplished microsurgically using a micropipette to remove the polar body and the adjacent cytoplasm. Oocytes can then be screened to identify those of which have been successfully enucleated. One way to screen oocytes can be to stain the oocytes with or with about 3-10 microgram per milliliter 33342 Hoechst dye in suitable holding medium, and then view the oocytes under ultraviolet irradiation for less than 10 seconds. Oocytes that have been successfully enucleated can then be placed in a suitable culture medium, for example, CRlaa plus 10% serum. The handling of oocytes can also be optimized for nuclear transfer.

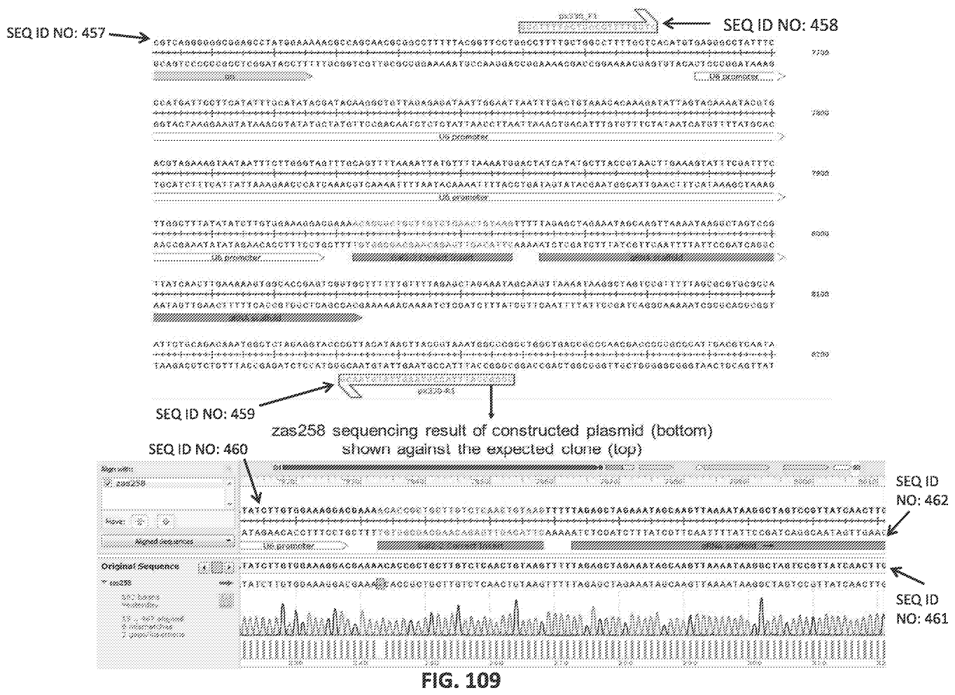

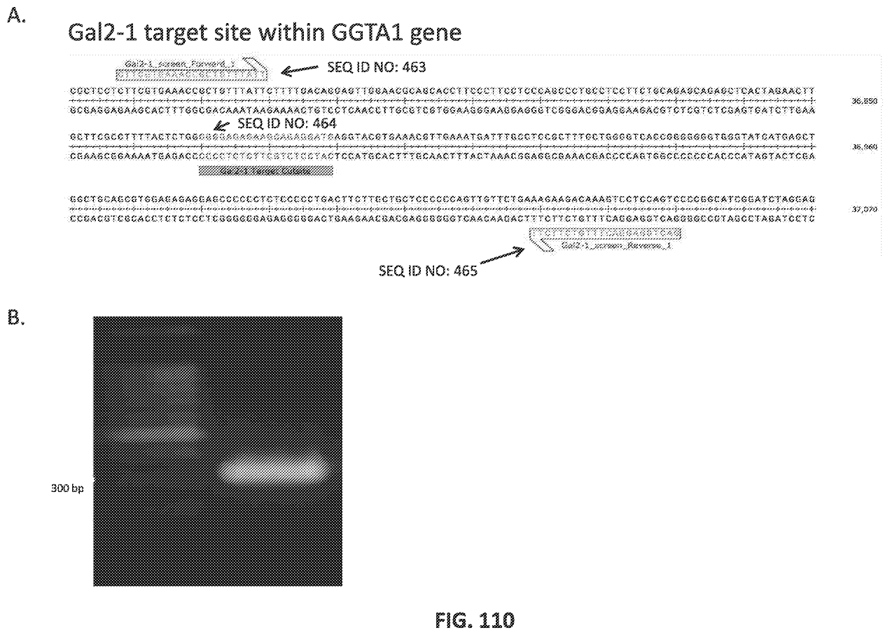

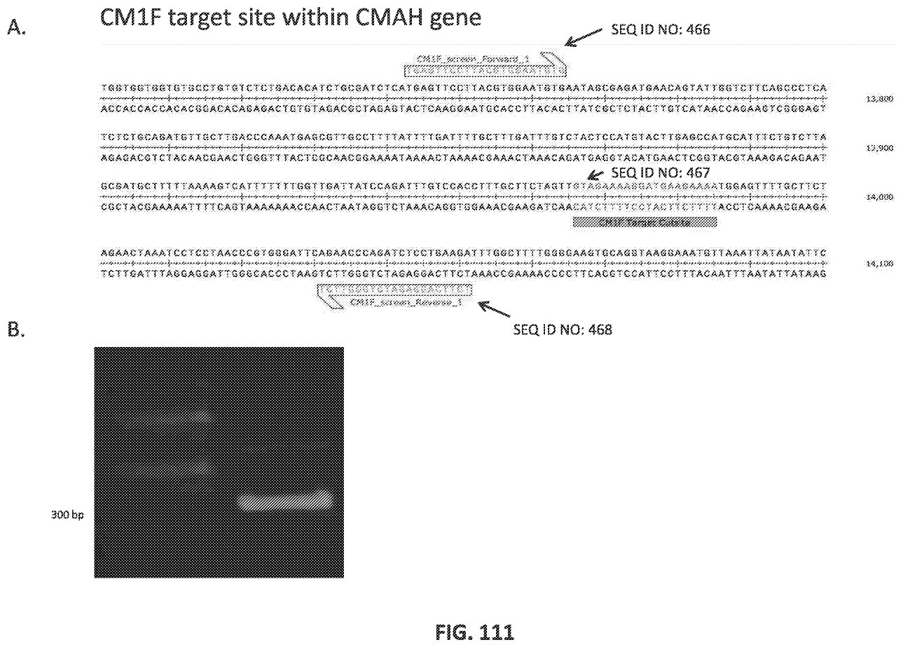

The embryos generated herein can be transferred to surrogate non-human animals (e.g., pigs) to produce offspring (e.g., piglets). For example, the embryos can be transferred to the oviduct of recipient gilts on the day or 1 day after estrus e.g., following mid-line laparotomy under general anesthesia. Pregnancy can be diagnosed, e.g., by ultrasound. Pregnancy can be diagnosed after or after about 28 days from the transfer. The pregnancy can then checked at or at about 2-week intervals by ultrasound examination. All of the microinjected offspring (e.g., piglets) can be delivered by natural birth. Information of the pregnancy and delivery (e.g., time of pregnancy, rates of pregnancy, number of offspring, survival rate, etc.) can be documented. The genotypes and phenotypes of the offspring can be measured using any methods described through the application such as sequencing (e.g., next-generation sequencing). Sequencing can also be Zas 258 sequencing, as shown in FIG. 109 and FIG. 110 A. Sequencing products can also be verified by electrophoresis of the amplification product, FIG. 110 B. For example, the CM1F sequencing is shown in FIG. 111 A and the electrophoresis product is shown in FIG. 111 B.

Cultured cells can be used immediately for nuclear transfer (e.g., somatic cell nuclear transfer), embryo transfer, and/or inducing pregnancy, allowing embryos derived from stable genetic modifications give rise to offspring (e.g., piglets). Such approach can reduce time and cost, e.g., months of costly cell screening that may result in genetically modified cells fail to produce live and/or healthy piglets.

Embryo growing and transferring can be performed using standard procedures used in the embryo growing and transfer industry. For example, surrogate mothers can be used. Embryos can also be grown and transferred in culture, for example, by using incubators. In some cases, an embryo can be transferred to an animal, e.g., a surrogate animal, to establish a pregnancy.

It can be desirable to replicate or generate a plurality of genetically modified non-human animals that have identical genotypes and/or phenotypes disclosed herein. For example, a genetically modified non-human animal can be replicated by breeding (e.g., selective breeding). A genetically modified non-human animal can be replicated by nuclear transfer (e.g., somatic cell nuclear transfer) or introduction of DNA into a cell (e.g., oocytes, sperm, zygotes or embryonic stem cells). These methods can be reproduced a plurality of times to replicate or generate a plurality of a genetically modified non-human animal disclosed herein. In some cases, cells can be isolated from the fetuses of a pregnant genetically modified non-human animal. The isolated cells (e.g., fetal cells) can be used for generating a plurality of genetically modified non-human animals similar or identical to the pregnant animal. For example, the isolated fetal cells can provide donor nuclei for generating genetically modified animals by nuclear transfer, (e.g., somatic cell nuclear transfer).

V. Methods of Use

Cells, organs, and/or tissues can be extracted from a non-human animal as described herein. Cells, organs, and/or tissues can be genetically altered ex vivo and used accordingly. These cells, organs, and/or tissues can be used for cell-based therapies. These cells, organs, and/or tissues can be used to treat or prevent disease in a recipient (e.g., a human or non-human animal). Surprisingly, the genetic modifications as described herein can help prevent rejection. Additionally, cells, organs, and/or tissues can be made into tolerizing vaccines to also help tolerize the immune system to transplantation. Further, tolerizing vaccines can temper the immune system, including, abrogating autoimmune responses.

Disclosed herein are methods for treating a disease in a subject in need thereof can comprise administering a tolerizing vaccine to the subject; administering a pharmaceutical agent that inhibits T cell activation to the subject; and transplanting a genetically modified cell to the subject. The pharmaceutical agent that inhibits T cell activation can be an antibody. The antibody can be an anti-CD40 antibody disclosed herein. The anti-CD40 antibody can be an antagonistic antibody. The anti-CD40 antibody can be an anti-CD40 antibody that specifically binds to an epitope within the amino acid sequence: EPPTACREKQYLINSQCCSLCQPGQKLVSDCTEFTETECLPCGESEFLDTWNRETHCHQH KYCDPNLGLRVQQKGTSETDTICTCEEGWHCTSEACESCV (SEQ ID NO: 487). The anti-CD40 antibody can be an anti-CD40 antibody that specifically binds to an epitope within the amino acid sequence: EKQYLINSQCCSLCQPGQKLVSDCTEFTETECL (SEQ ID NO: 488). The anti-CD40 antibody can be a Fab′ anti-CD40L monoclonal antibody fragment CDP7657. The anti-CD-40 antibody can be a FcR-engineered, Fc silent anti-CD40L monoclonal domain antibody. The cell transplanted to the subject can be any genetically modified cell described throughout the application. The tissue or organ transplanted to the subject can comprise one or more of the genetically modified cells. In some cases, the methods can further comprise administering one or more immunosuppression agent described in the application, such as further comprising providing to the recipient one or more of a B-cell depleting antibody, an mTOR inhibitor, a TNF-alpha inhibitor, a IL-6 inhibitor, a nitrogen mustard alkylating agent (e.g., cyclophosphamide), and a complement C3 or C5 inhibitor.

Also disclosed herein are methods for treating a disease, comprising transplanting one or more cells to a subject in need thereof. The one or more cells can be any genetically modified cells disclosed herein. In some cases, the methods can comprise transplanting a tissue or organ comprising the one or more cells (e.g., genetically modified cells) to the subject in need thereof.

Described herein are methods of treating or preventing a disease in a recipient (e.g., a human or non-human animal) comprising transplanting to the recipient (e.g., a human or non-human animal) one or more cells (including organs and/or tissues) derived from a genetically modified non-human animal comprising one or more genes with reduced expression. One or more cells can be derived from a genetically modified non-human animal as described throughout.

The methods disclosed herein can be used for treating or preventing disease including, but not limited to, diabetes, cardiovascular diseases, lung diseases, liver diseases, skin diseases, or neurological disorders. For example, the methods can be used for treating or preventing Parkinson's disease or Alzheimer's disease. The methods can also be used for treating or preventing diabetes, including type 1, type 2, cystic fibrosis related, surgical diabetes, gestational diabetes, mitochondrial diabetes, or combination thereof. In some cases, the methods can be used for treating or preventing hereditary diabetes or a form of hereditary diabetes. Further, the methods can be used for treating or preventing type 1 diabetes. The methods can also be used for treating or preventing type 2 diabetes. The methods can be used for treating or preventing pre-diabetes.

For example, when treating diabetes, genetically modified splenocytes can be fixed with ECDI and given to a recipient. Further, genetically modified pancreatic islet cells can be grafted into the same recipient to produce insulin. Genetically modified splenocytes and pancreatic islet cells can be genetically identical and can also be derived from the same genetically modified non-human animal.

Provided herein include i) genetically modified cells, tissues or organs for use in administering to a subject in need thereof to treat a condition in the subject; ii) a tolerizing vaccine for use in immunotolerizing the subject to a graft, where the tolerizing vaccine comprise a genetically modified cell, tissue, or organ; iii) one or more pharmaceutical agents for use in inhibiting T cell activation, B cell activation, dendritic cell activation, or a combination thereof in the subject; or iv) any combination thereof.

Also provided herein include genetically modified cells, tissues or organs for use in administering to a subject in need thereof to treat a condition in the subject. The subject can have been or become tolerized to the genetically modified cell, tissue or organ by use of a tolerizing vaccine. Further, the subject can be administered one or more pharmaceutical agents that inhibit T cell activation, B cell activation, dendritic cell activation, or a combination thereof.

Transplantation

The methods disclosed herein can comprise transplanting. Transplanting can be autotransplanting, allotransplanting, xenotransplanting, or any other transplanting. For example, transplanting can be xenotransplanting. Transplanting can also be allotransplanting. “Xenotransplantation” and its grammatical equivalents as used herein can encompass any procedure that involves transplantation, implantation, or infusion of cells, tissues, or organs into a recipient, where the recipient and donor are different species. Transplantation of the cells, organs, and/or tissues described herein can be used for xenotransplantation in into humans. Xenotransplantation includes but is not limited to vascularized xenotransplant, partially vascularized xenotransplant, unvascularized xenotransplant, xenodressings, xenobandages, and nanostructures.

“Allotransplantation” and its grammatical equivalents as used herein can encompass any procedure that involves transplantation, implantation, or infusion of cells, tissues, or organs into a recipient, where the recipient and donor are the same species. Transplantation of the cells, organs, and/or tissues described herein can be used for allotransplantation in into humans. Allotransplantation includes but is not limited to vascularized allotransplant, partially vascularized allotransplant, unvascularized allotransplant, allodressings, allobandages, and allostructures.

After treatment (e.g., any of the treatment as disclosed herein), transplant rejection can be improved as compared to when one or more wild-type cells is transplanted into a recipient. For example, transplant rejection can be hyperacute rejection. Transplant rejection can also be acute rejection. Other types of rejection can include chronic rejection. Transplant rejection can also be cell-mediated rejection or T cell-mediated rejection. Transplant rejection can also be natural killer cell-mediated rejection.

In some cases, a subject is sensitized to major histocompatibility complex (MHC) or human leukocyte antigen (HLA). For example, a subject may have a positive result on a panel reactive antibody (PRA) screen. In some cases, a subject may have a calculated PRA (cPRA) score from 0.1 to 100%. A cPRA score can be or can be about from 0.1 to 10%, 5% to 30%, 10% to 50%, 20% to 80%, 40% to 90%, 50% to 100%. In some cases, a subject with a positive PRA screen may be transplanted with the genetically modified cells of the invention.

In some cases, a subject may have a quantification performed of their PRA level by a single antigen bead (SAB) test. An SAB test can identify MHC or HLA for which a subject has antibodies to.

“Improving” and its grammatical equivalents as used herein can mean any improvement recognized by one of skill in the art. For example, improving transplantation can mean lessening hyperacute rejection, which can encompass a decrease, lessening, or diminishing of an undesirable effect or symptom.

The disclosure describes methods of treatment or preventing diabetes or prediabetes. For example, the methods include but are not limited to, administering one or more pancreatic islet cell(s) from a donor non-human animal described herein to a recipient, or a recipient in need thereof. The methods can be transplantation or, in some cases, xenotransplantation. The donor animal can be a non-human animal. A recipient can be a primate, for example, a non-human primate including, but not limited to, a monkey. A recipient can be a human and in some cases, a human with diabetes or pre-diabetes. In some cases, whether a patient with diabetes or pre-diabetes can be treated with transplantation can be determined using an algorithm, e.g., as described in Diabetes Care 2015; 38:1016-1029, which is incorporated herein by reference in its entirety.

The methods can also include methods of xenotransplantation where the transgenic cells, tissues and/or organs, e.g., pancreatic tissues or cells, provided herein are transplanted into a primate, e.g., a human, and, after transplant, the primate requires less or no immunosuppressive therapy. Less or no immunosuppressive therapy includes, but is not limited to, a reduction (or complete elimination of) in dose of the immunosuppressive drug(s)/agent(s) compared to that required by other methods; a reduction (or complete elimination of) in the number of types of immunosuppressive drug(s)/agent(s) compared to that required by other methods; a reduction (or complete elimination of) in the duration of immunosuppression treatment compared to that required by other methods; and/or a reduction (or complete elimination of) in maintenance immunosuppression compared to that required by other methods.

The methods disclosed herein can be used for treating or preventing disease in a recipient (e.g., a human or non-human animal). A recipient can be any non-human animal or a human. For example, a recipient can be a mammal. Other examples of recipient include but are not limited to primates, e.g., a monkey, a chimpanzee, a bamboo, or a human. If a recipient is a human, the recipient can be a human in need thereof. The methods described herein can also be used in non-primate, non-human recipients, for example, a recipient can be a pet animal, including, but not limited to, a dog, a cat, a horse, a wolf, a rabbit, a ferret, a gerbil, a hamster, a chinchilla, a fancy rat, a guinea pig, a canary, a parakeet, or a parrot. If a recipient is a pet animal, the pet animal can be in need thereof. For example, a recipient can be a dog in need thereof or a cat in need thereof.

Transplanting can be by any transplanting known to the art. Graft can be transplanted to various sites in a recipient. Sites can include, but not limited to, liver subcapsular space, splenic subcapsular space, renal subcapsular space, omentum, bursa omentalis, gastric or intestinal submucosa, vascular segment of small intestine, venous sac, testis, brain, spleen, or cornea. For example, transplanting can be subcapsular transplanting. Transplanting can also be intramuscular transplanting. Transplanting can be intraportal transplanting.

Transplanting can be of one or more cells, tissues, and/or organs from a human or non-human animal. For example, the tissue and/or organs can be, or the one or more cells can be from, a brain, heart, lungs, eye, stomach, pancreas, kidneys, liver, intestines, uterus, bladder, skin, hair, nails, ears, glands, nose, mouth, lips, spleen, gums, teeth, tongue, salivary glands, tonsils, pharynx, esophagus, large intestine, small intestine, rectum, anus, thyroid gland, thymus gland, bones, cartilage, tendons, ligaments, suprarenal capsule, skeletal muscles, smooth muscles, blood vessels, blood, spinal cord, trachea, ureters, urethra, hypothalamus, pituitary, pylorus, adrenal glands, ovaries, oviducts, uterus, vagina, mammary glands, testes, seminal vesicles, penis, lymph, lymph nodes or lymph vessels. The one or more cells can also be from a brain, heart, liver, skin, intestine, lung, kidney, eye, small bowel, or pancreas. The one or more cells are from a pancreas, kidney, eye, liver, small bowel, lung, or heart. The one or more cells can be from a pancreas. The one or more cells can be pancreatic islet cells, for example, pancreatic β cells. Further, the one or more cells can be pancreatic islet cells and/or cell clusters or the like, including, but not limited to pancreatic α cells, pancreatic β cells, pancreatic δ cells, pancreatic F cells (e.g., PP cells), or pancreatic ε cells. In one instance, the one or more cells can be pancreatic α cells. In another instance, the one or more cells can be pancreatic β cells.

As discussed above, a genetically modified non-human animal can be used in xenograft (e.g., cells, tissues and/or organ) donation. Solely for illustrative purposes, genetically modified non-human animals, e.g., pigs, can be used as donors of pancreatic tissue, including but not limited to, pancreatic islets and/or islet cells. Pancreatic tissue or cells derived from such tissue can comprise pancreatic islet cells, or islets, or islet-cell clusters. For example, cells can be pancreatic islets which can be transplanted. More specifically, cells can be pancreatic β cells. Cells also can be insulin-producing. Alternatively, cells can be islet-like cells. Islet cell clusters can include any one or more of α, β, δ, PP or ε cells. A disease to be treated by methods and compositions herein can be diabetes. Transplantable grafts can be pancreatic islets and/or cells from pancreatic islets. A modification to a transgenic animal can be to the pancreatic islets or cells from pancreatic islets. In some cases, pancreatic islets or cells from a pancreatic islet can be porcine. In some cases, cells from a pancreatic islet include pancreatic β cells.

Donor non-human animals can be at any stage of development including, but not limited to, embryonic, fetal, neonatal, young and adult. For example, donor cells islet cells can be isolated from adult non-human animals. Donor cells, e.g., islet cells, can also be isolated from fetal or neonatal non-human animals. Donor non-human animals can be under the age of 10, 9, 8, 7, 6, 5, 4, 3, 2, or 1 year(s). For example, islet cells can be isolated from a non-human animal under the age of 6 years. Islet cells can also be isolated from a non-human animal under the age of 3 years. Donors can be non-human animals and can be any age from or from about 0 (including a fetus) to 2; 2 to 4; 4 to 6; 6 to 8; or 8 to 10 years. A non-human animal can be older than or than about 10 years. Donor cells can be from a human as well.

Islet cells can be isolated from non-human animals of varying ages. For example, islet cells can be isolated from or from about newborn to 2 year old non-human animals. Islets cells can also be isolated from or from about fetal to 2 year old non-human animals. Islets cells can be isolated from or from about 6 months old to 2 year old non-human animals. Islets cells can also be isolated from or from about 7 months old to 1 year old non-human animals. Islets cells can be isolated from or from about 2-3 year old non-human animals. In some cases, non-human animals can be less than 0 years (e.g., a fetus or embryo). In some cases, neonatal islets can be more hearty and consistent post-isolation than adult islets, can be more resistant to oxidative stress, can exhibit significant growth potential (likely from a nascent islet stem cell subpopulation), such that they can have the ability to proliferate post-transplantation and engraftment in a transplantation site.

With regards to treating diabetes, neonatal islets can have the disadvantage that it can take them up to or up to about 4-6 weeks to mature enough such that they produce significant levels of insulin, but this can be overcome by treatment with exogenous insulin for a period sufficient for the maturation of the neonatal islets. In xenograft transplantation, survival and functional engraftment of neo-natal islets can be determined by measuring donor-specific c-peptide levels, which are easily distinguished from any recipient endogenous c-peptide.

As discussed above, adult cells can be isolated. For example, adult non-human animal islets, e.g., adult porcine cells, can be isolated. Islets can then be cultured for or for about 1-3 days prior to transplantation in order to deplete the preparation of contaminating exocrine tissue. Prior to treatment, islets can be counted, and viability assessed by double fluorescent calcein-AM and propidium iodide staining. Islet cell viability >75% can be used. However, cell viability greater than or greater than about 40%, 50%, 60%, 70%, 80%, 90%, 95%, 99% can be used. For example, cells that exhibit viability from or from about 40% to 50%; 50% to 60%; 60% to 70%; 70% to 80%; 80% to 90%; 90% to 95%, or 90% to 100% can be used. Additionally, purity can be greater than or greater than about 80% islets/whole tissue. Purity can also be at least or at least about 40%, 50%, 60%, 70%, 80%, 90%, 95%, or 99% islets/whole tissue. For example, purity can be from or can be from about 40% to 50%; 50% to 60%; 60% to 70%; 70% to 80%; 80% to 90%; 90% to 100%; 90% to 95%, or 95% to 100%.

Functional properties of islets, including glucose-stimulated insulin secretion as assed by dynamic perifusion and viability, can be determined in vitro prior to treatment (Balamurugan, 2006). For example, non-human animal islet cells, e.g., transgenic porcine islet cells can be cultured in vitro to expand, mature, and/or purify them so that they are suitable for grafting.

Islet cells can also be isolated by standard collagenase digestion of minced pancreas. For example, using aseptic techniques, glands can be distended with tissue dissociating enzymes (a mixture of purified enzymes formulated for rapid dissociation of a pancreas and maximal recovery of healthy, intact, and functional islets of Langerhans, where target substrates for these enzymes are not fully identified, but are presumed to be collagen and non-collagen proteins, which comprise intercellular matrix of pancreatic acinar tissue) (1.5 mg/ml), trimmed of excess fat, blood vessels and connective tissue, minced, and digested at 37 degree C. in a shaking water bath for 15 minutes at 120 rpm. Digestion can be achieved using lignocaine mixed with tissue dissociating enzymes to avoid cell damage during digestion. Following digestion, the cells can be passed through a sterile 50 mm to 1000 mm mesh, e.g., 100 mm, 200 mm, 300 mm, 400 mm, 500 mm, 600 mm, 700 mm, 800 mm, 900 mm, or 1000 mm mesh into a sterile beaker. Additionally, a second digestion process can be used for any undigested tissue.

Islets can also be isolated from the adult pig pancreas (Brandhorst et al., 1999). The pancreas is retrieved from a suitable source pig, pen-pancreatic tissue is removed, the pancreas is divided into the splenic lobe and in the duodenal/connecting lobe, the ducts of each lobes are cannulated, and the lobes are distended with tissue dissociating enzymes. The pancreatic lobes are placed into a Ricordi chamber, the temperature is gradually increased to 28 to 32° C., and the pancreatic lobes are dissociated by means of enzymatic activity and mechanical forces. Liberated islets are separated from acinar and ductal tissue using continuous density gradients. Purified pancreatic islets are cultured for or for about 2 to 7 days, subjected to characterization, and islet products meeting all specifications are released for transplantation (Korbutt et al., 2009).

Donor cells, organs, and/or tissues before, after, and/or during transplantation can be functional. For example, transplanted cells, organs, and/or tissues can be functional for at least or at least about 1, 5, 10, 20, 30 days after transplantation. Transplanted cells, organs, and/or tissues can be functional for at least or at least about 1, 2, 3, 4, 5, 6, 7, 8, 9, 10, 11, or 12 months after transplantation. Transplanted cells, organs, and/or tissues can be functional for at least or at least about 1, 2, 3, 4, 5, 6, 7, 8, 9, 10, 15, 20, 25, or 30 years after transplantation. In some cases, transplanted cells, organs, and/or tissues can be functional for up to the lifetime of a recipient. This can indicate that transplantation was successful. This can also indicate that there is no rejection of the transplanted cells, tissues, and/or organs.

Further, transplanted cells, organs, and/or tissues can function at 100% of its normal intended operation. Transplanted cells, organs, and/or tissues can also function at least or at least about 50, 60, 65, 70, 75, 80, 85, 90, 95, 99, or 100% of its normal intended operation, e.g., from or from about 50 to 60; 60 to 70; 70 to 80; 80 to 90; 90 to 100%. In certain instances, the transplanted cells, organs, and/or tissues can function at greater 100% of its normal intended operation (when compared to a normal functioning non-transplanted cell, organ, or tissue as determined by the American Medical Association). For example, the transplanted cells, organs, and/or tissues can function at or at about 110, 120, 130, 140, 150, 175, 200% or greater of its normal intended operation, e.g., from or from about 100 to 125; 125 to 150; 150 to 175; 175 to 200%.

In certain instances, transplanted cells can be functional for at least or at least about 1 day. Transplanted cells can also functional for at least or at least about 7 days. Transplanted cells can be functional for at least or at least about 14 days. Transplanted cells can be functional for at least or at least about 21 days. Transplanted cells can be functional for at least or at least about 28 days. Transplanted cells can be functional for at least or at least about 60 days.

Another indication of successful transplantation can be the days a recipient does not require immunosuppressive therapy. For example, after treatment (e.g., transplantation) provided herein, a recipient can require no immunosuppressive therapy for at least or at least about 1, 5, 10, 100, 365, 500, 800, 1000, 2000, 4000 or more days. This can indicate that transplantation was successful. This can also indicate that there is no rejection of the transplanted cells, tissues, and/or organs.

In some cases, a recipient can require no immunosuppressive therapy for at least or at least about 1 day. A recipient can also require no immunosuppressive therapy for at least or at least about 7 days. A recipient can require no immunosuppressive therapy for at least or at least about 14 days. A recipient can require no immunosuppressive therapy for at least or at least about 21 days. A recipient can require no immunosuppressive therapy for at least or at least about 28 days. A recipient can require no immunosuppressive therapy for at least or at least about 60 days. Furthermore, a recipient can require no immunosuppressive therapy for at least or at least about 1, 2, 3, 4, 5, 6, 7, 8, 9, 10, 15, 20, 30, 40, or 50 years, e.g., for at least or at least about 1 to 2; 2 to 3; 3 to 4; 4 to 5; 1 to 5; 5 to 10; 10 to 15; 15 to 20; 20 to 25; 25 to 50 years.

Another indication of successful transplantation can be the days a recipient requires reduced immunosuppressive therapy. For example, after the treatment provided herein, a recipient can require reduced immunosuppressive therapy for at least or at least about 1, 5, 10, 50, 100, 200, 300, 365, 400, 500 days, e.g., for at least or at least about 1 to 30; 30 to 120; 120 to 365; 365 to 500 days. This can indicate that transplantation was successful. This can also indicate that there is no or minimal rejection of the transplanted cells, tissues, and/or organs.

For example, a recipient can require reduced immunosuppressive therapy for at least or at least about 1 day. A recipient can also require reduced immunosuppressive therapy for at least 7 days. A recipient can require reduced immunosuppressive therapy for at least or at least about 14 days. A recipient can require reduced immunosuppressive therapy for at least or at least about 21 days. A recipient can require reduced immunosuppressive therapy for at least or at least about 28 days. A recipient can require reduced immunosuppressive therapy for at least or at least about 60 days. Furthermore, a recipient can require reduced immunosuppressive therapy for at least or at least about 1, 2, 3, 4, 5, 6, 7, 8, 9, 10, 15, 20, 30, 40, or 50 years, e.g., for at least or at least about 1 to 2; 2 to 3; 3 to 4; 4 to 5; 1 to 5; 5 to 10; 10 to 15; 15 to 20; 20 to 25; 25 to 50 years.

“Reduced” and its grammatical equivalents as used herein can refer to less immunosuppressive therapy compared to a required immunosuppressive therapy when one or more wild-type cells is transplanted into a recipient.

A donor (e.g., a donor for a transplant graft and/or a cell in a tolerizing vaccine) can be a mammal. A donor of allografts can be an unmodified human cell, tissue, and/or organ, including but not limited to pluripotent stem cells. A donor of xenografts can be any cell, tissue, and/or organ from a non-human animal, such as a mammal. In some cases, the mammal can be a pig.

The methods herein can further comprise treating a disease by transplanting one or more donor cells to an immunotolerized recipient (e.g., a human or a non-human animal).

### EXAMPLES

### Example 1: Generating Plasmids Expressing Guide RNA for Disrupting GGTA1, CMAH, NLRC5, B4GALNT2, and/or C3 Genes in Pigs

Genetically modified pigs will provide transplant grafts that induce low or no immuno-rejection in a recipient, and/or cells as tolerizing vaccines that enhance immuno-tolerization in the recipient. Such pigs will have reduced expression of any genes that regulate MHC molecules (e.g., MHC I molecules and/or MHC II molecules) compared to a non-genetically modified counterpart animal. Reducing expression of such genes will result in reduced expression and/or function of MHC molecules. These genes will be one or more of the following: components of an MHC I-specific enhanceosome, transporters of a MHC I-binding peptide, natural killer group 2D ligands, CXCR 3 ligands, C3, and CIITA. Additionally or alternatively, such pigs will comprise reduced protein expression of an endogenous gene that is not expressed in human (e.g., CMAH, GGTA1 and/or B4GALNT2). For example, the pigs will comprise reduced protein expression of one or more of the following: NLRC5, TAP1, C3, CXCL10, MICA, MICB, CIITA, CMAH, GGTA1 and/or B4GALNT2. In some cases, pigs will comprise reduced protein expression of NLRC5, C3, CXCL10, CMAH, GGTA1 and/or B4GALNT2.

This example shows exemplary methods for generating plasmids for disrupting GGTA1, CMAH, NLRC5, B4GALNT2, and/or C3 genes in pigs using the CRISPR/cas9 system. The plasmids were generated using the px330 vector, which simultaneously expressed a Cas9 DNA endonuclease and a guide RNA.

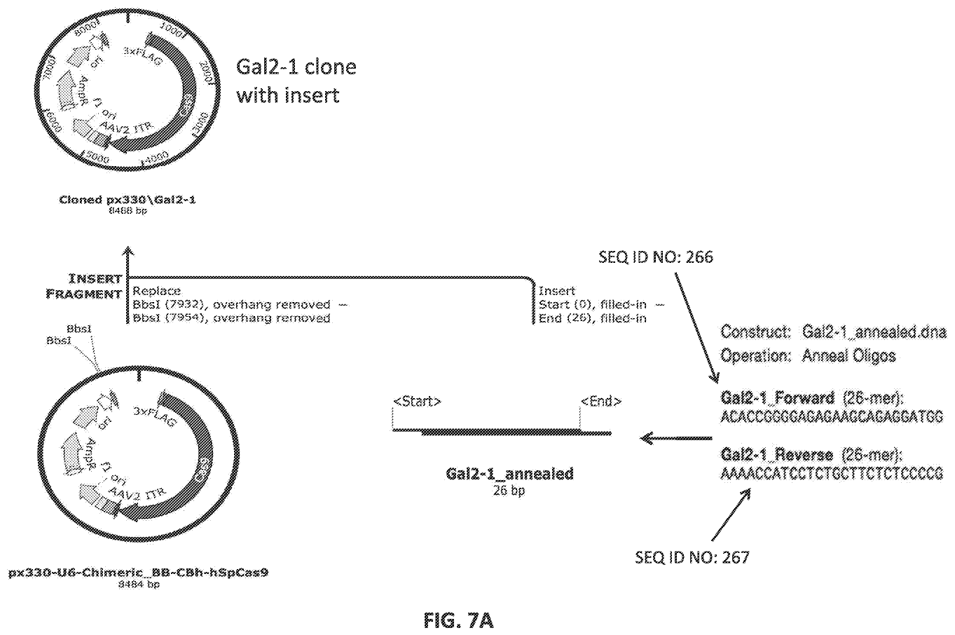

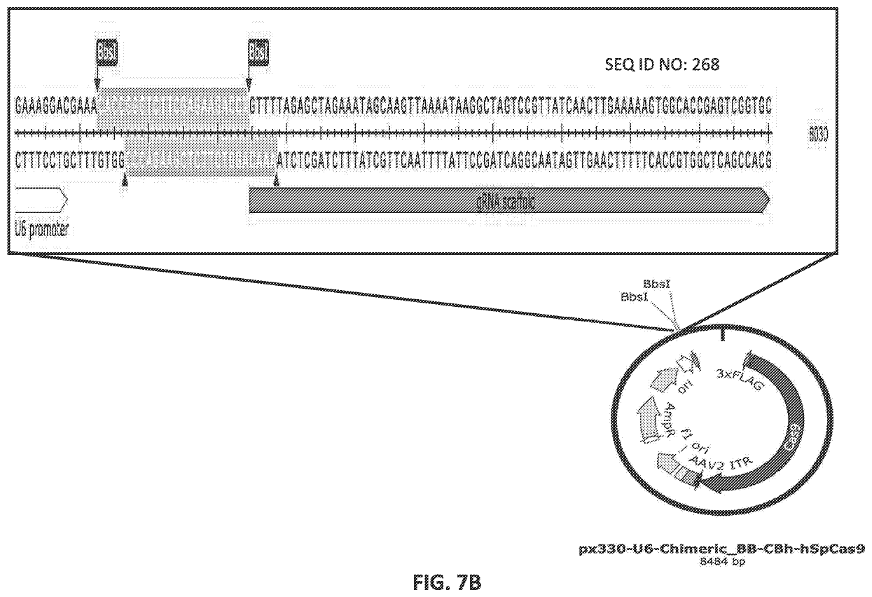

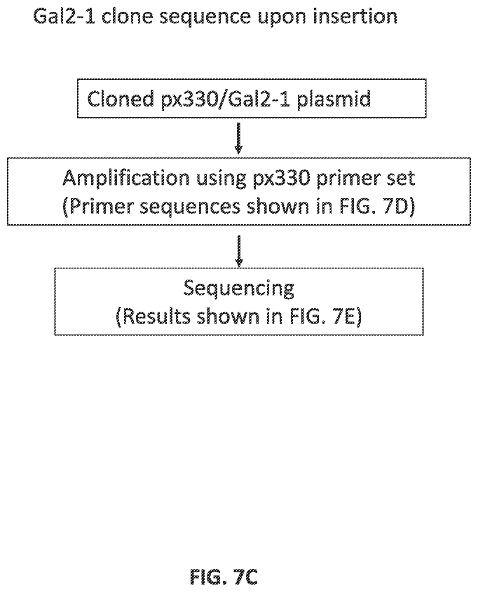

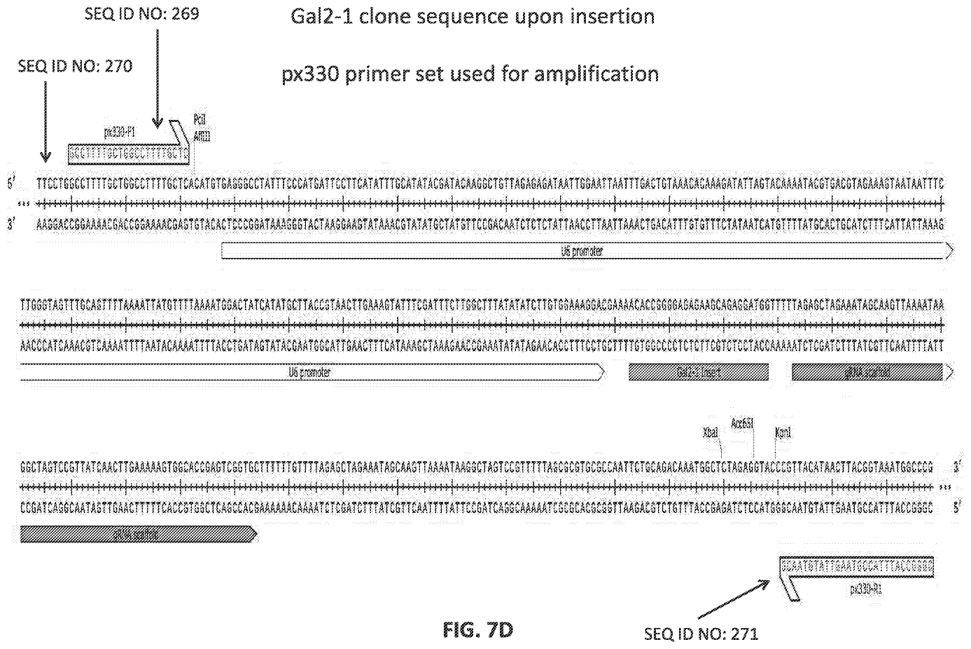

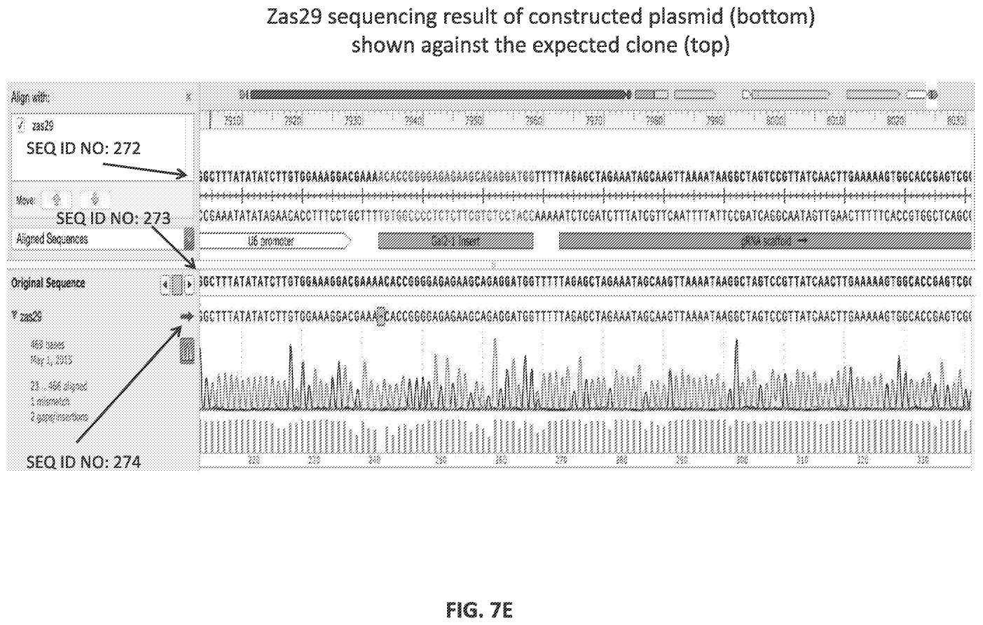

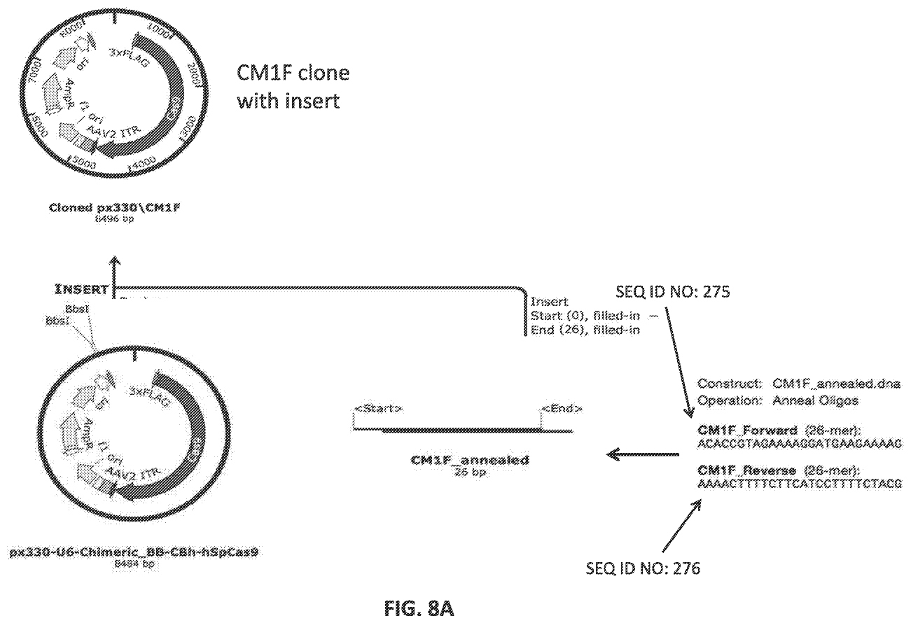

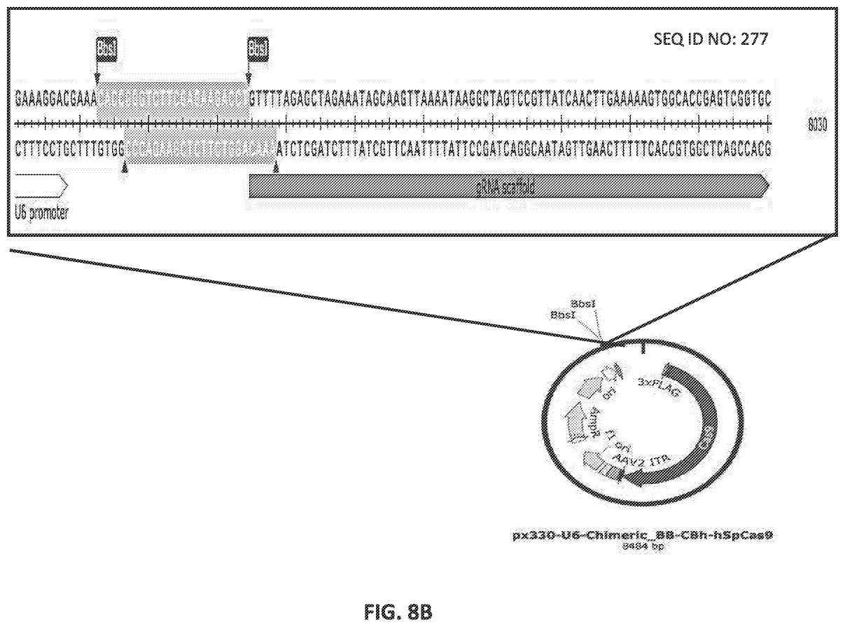

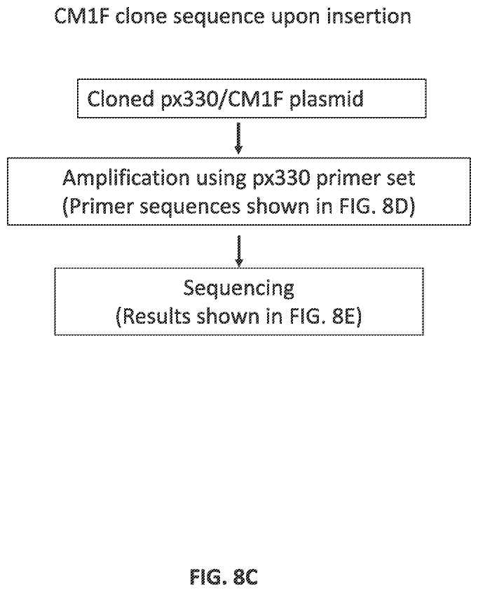

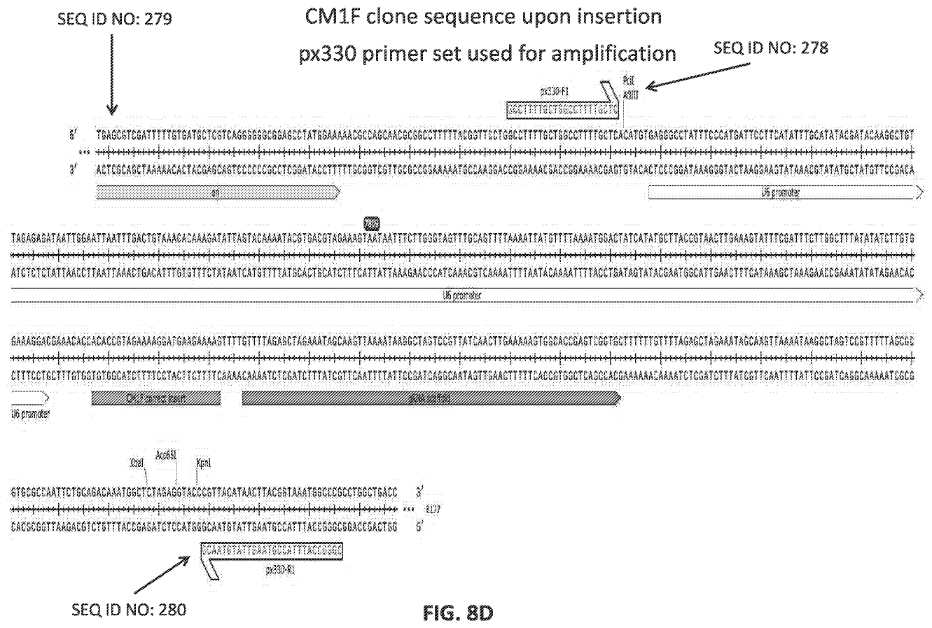

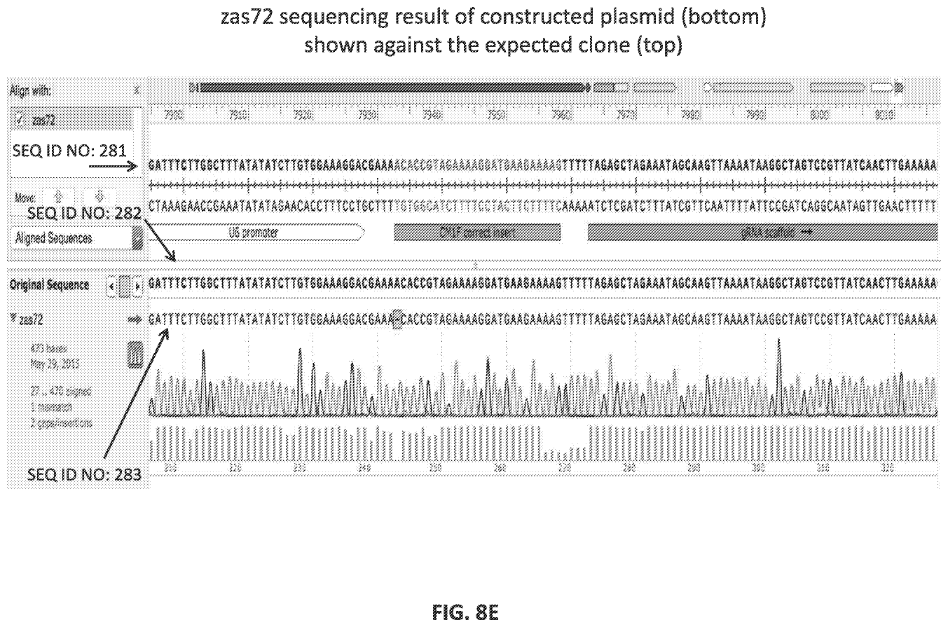

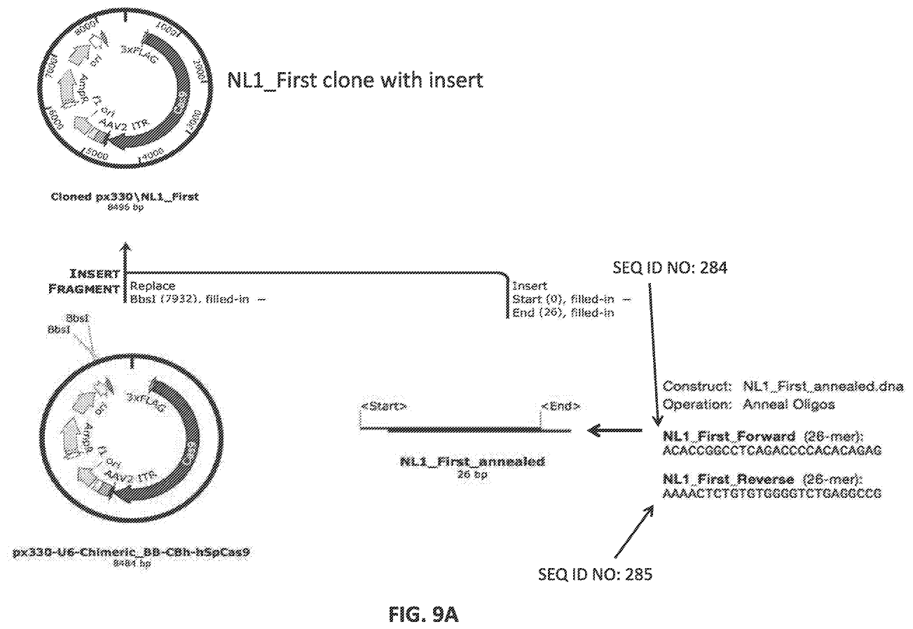

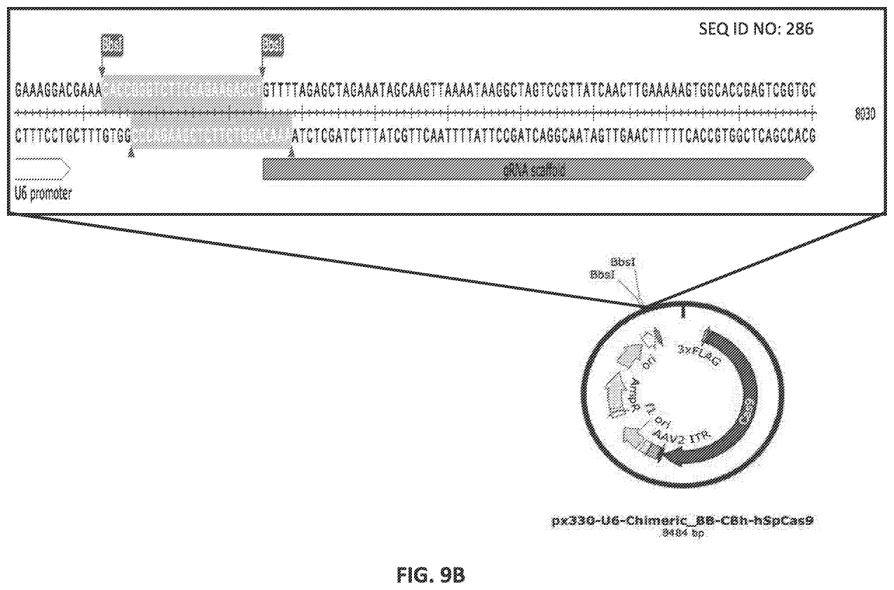

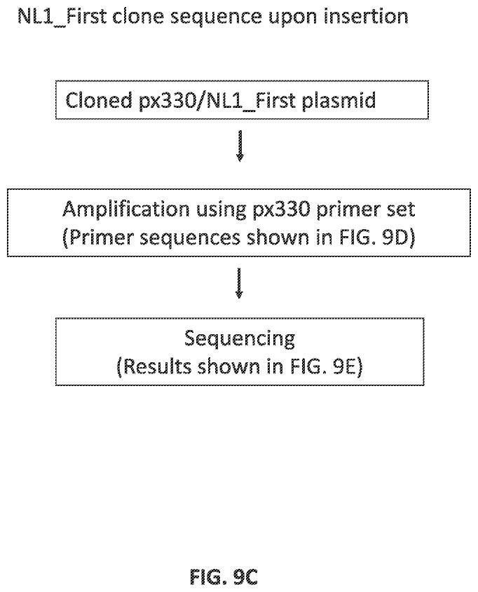

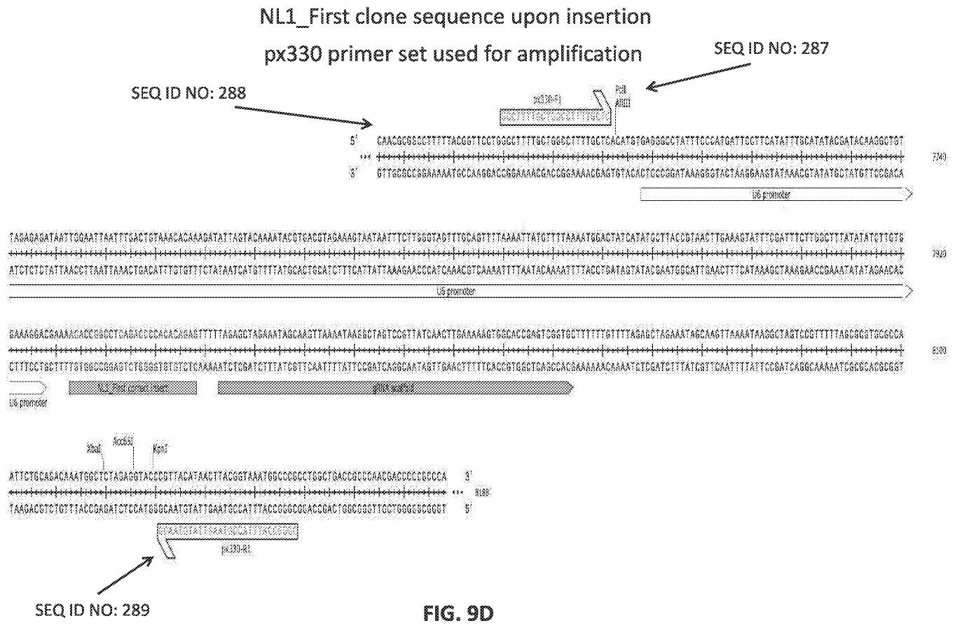

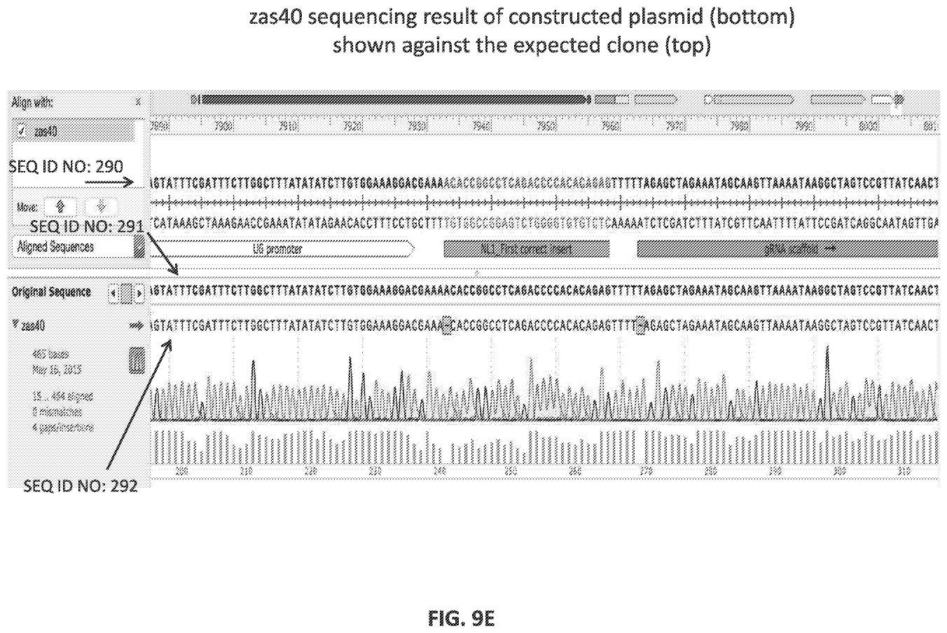

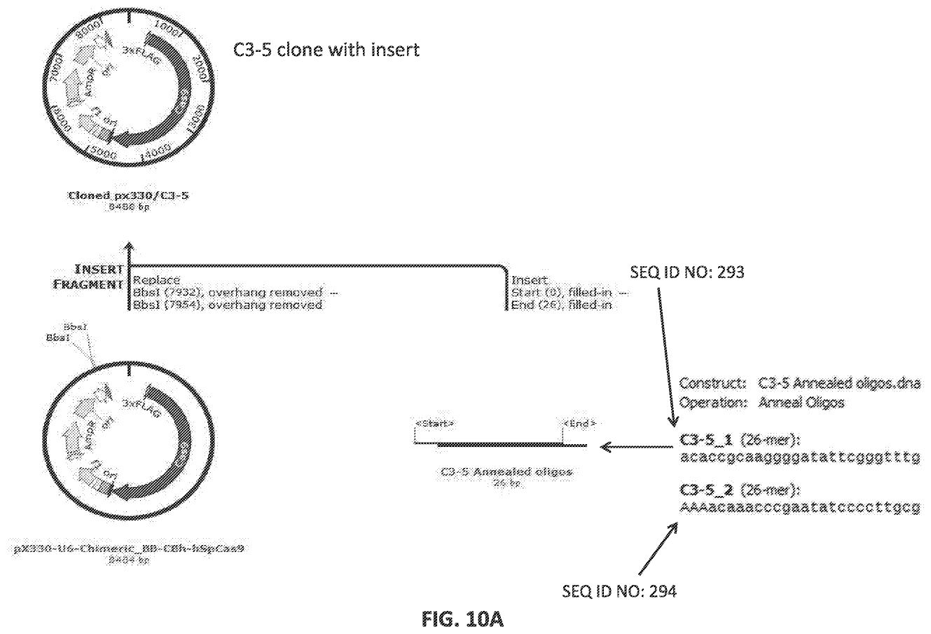

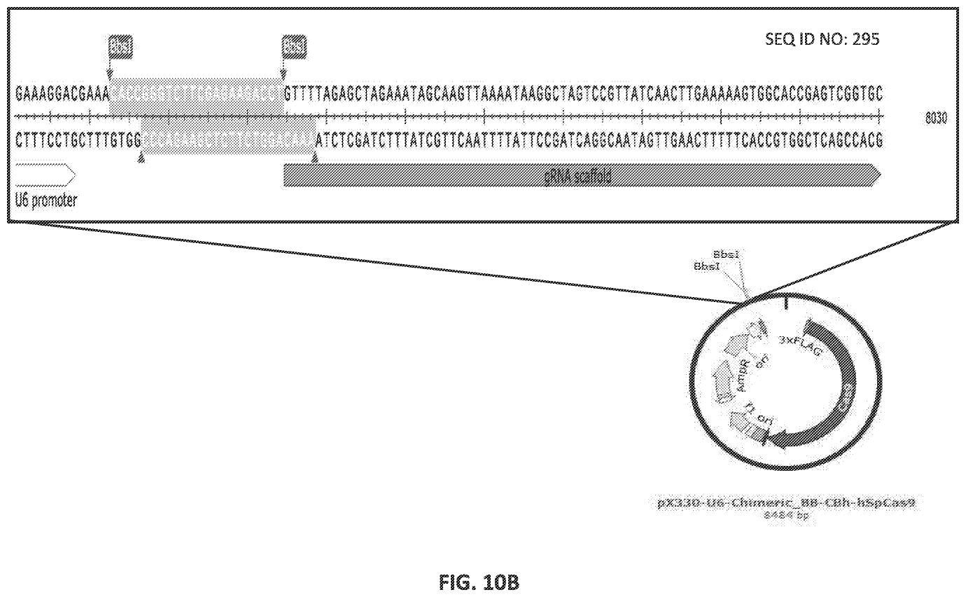

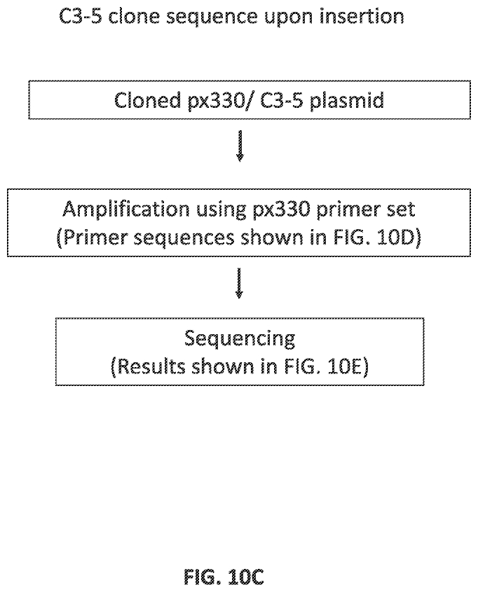

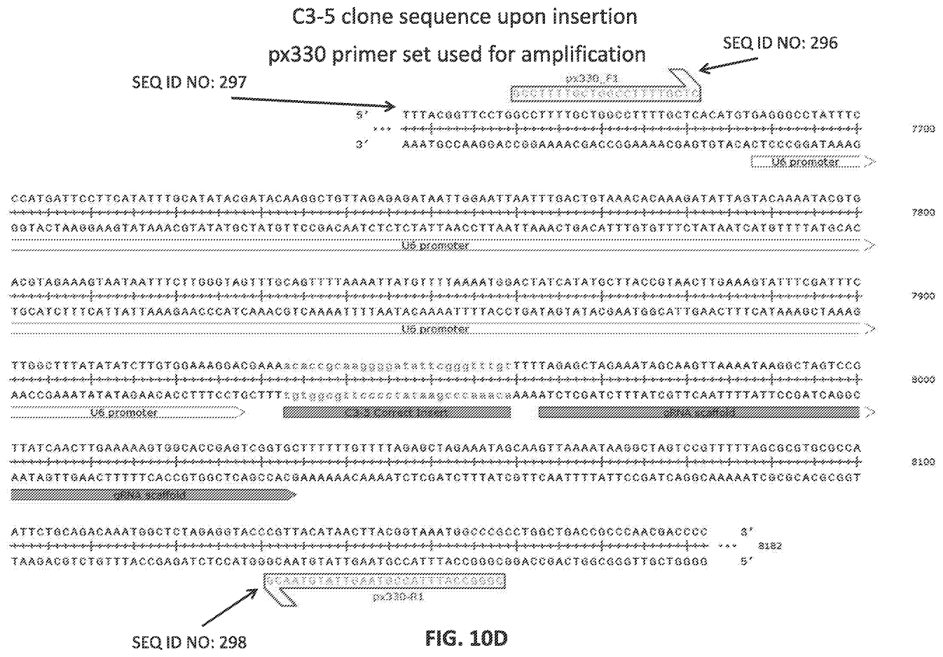

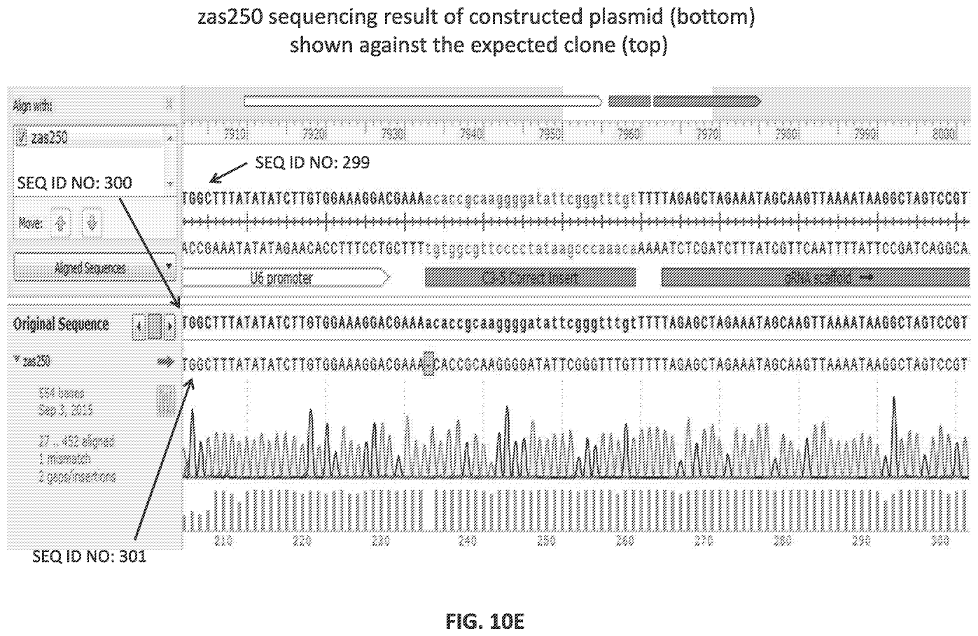

The px330-U6-Chimeric BB-CBh-hSpCas9 (#42230) plasmid was obtained from Addgene in a bacterial stab culture format. The stab culture was streaked onto a pre-warmed LB agar with ampicillin plate and incubated at 37° C. overnight. The next day, a single colony was selected and inoculated in a liquid LB overnight culture with ampicillin (5 mL for mini-prep, or 80-100 mL for maxi-prep). Mini-prep was performed using Qiagen kits according to manufacturer's instructions. Plasmid was eluted in nuclease free water and stocks were stored at −20° C. The oligonucleotides designed for targeting GGTA1, CMAH, NLRC5, C3, and B4GALNT2 are shown in Table 6. The oligonucleotides were synthesized by IDT. FIGS. 7A-7E, 8A-8E, 9A-9E, 10A-10E, and 11A-11E, show the cloning strategies for cloning plasmids targeting GGTA1 (i.e., px330/Gal2-1) (FIGS. 7A-7E), CMAH (i.e., px330/CM1F) (FIGS. 8A-8E), NLRC5 (i.e., px330/NL1_First) (FIGS. 9A-9E), C3 (i.e., px330/C3-5) (FIGS. 10A-10E), and B4GALNT2 (i.e., px330/B41_second) (FIGS. 11A-11E). The constructed px330 plasmids were validated by sequencing using sequencing primers shown in Table 7 and by sequencing as shown in FIG. 109. Oligonucleotides were re-suspended at 100 μM with nuclease free water and stored in the −20° C. freezer.

Vector digestion: The px330 vectors were digested in a reaction solution containing 5 μg px330 stock, 5 μL 10× FastDigest Reaction Buffer, 354 nuclease free water, and 5 μL FastDigest Bbsl enzyme (Cutsite: GAAGAC). The reaction solution was incubated at 37° C. for 15 minutes, the heat inactivated at 65° C. for 15 minutes. To desphosphorylate the vector, 0.24 (2 U; 1 U/1 pmol DNA ends) CIP was added and the resulting mixture was incubated at 37° C. for 60 minutes. The linearized plasmid was purified using Qiagen PCR Cleanup kit, and eluted with nuclease free water and stored at −20° C. until use.

Oligonucleotides Annealing and phosphorylation: a solution was made by mixing 1 μL 100 uM Forward oligonucleotide, 1 μL 100 uM Reverse oligonucleotide, 1 μL 10×T4 Ligase Buffer, 6 μL nuclease free water, 14 Polynucleotide Kinase (PNK). The resulting solution was incubated on a thermal cycler running the following program: 37° C. for 30 min, 95° C. for 5 min, ramp down to 25° C. at 0.1° C./second.

Ligation Reaction: a solution was made by mixing diluted annealed oligonucleotides 1:250 with nuclease free water, 2 μL diluted annealed oligonucleotides, 100 ng linearized/dephosphorylated px330 vector, 5 μL 10×T4 Ligase Buffer, nuclease free water to bring to 50 μL final volume, and 2.5 μL T4 DNA Ligase. The solution was incubated at room temp for 4 hours, then heat inactivated at 65° C. for 10 minutes.

Transformation: TOP10 E. coli vials were thawed from −80° C. freezer on ice for 15 minutes prior to transformation. 2 μL of the ligation reaction product was added to the cells and mixed by gently flicking the tubes. The tubes were incubated on ice for 5 minutes, heat shocked in 42° C. water bath for 30 seconds, and placed back on ice for additional 2 minutes after heat shock. 50 μL of transformed cells were plated onto an LB agar with ampicillin plate and spread with pipette tip. The plates were incubated at 37° C. overnight.

Colony PCR screening for correctly inserted oligonucleotides: 3× colonies were selected from the plate and labeled 1-3 on bottom of plate. Master mix for PCR reaction was prepared by mixing 15 μL 10× Standard Taq Reaction Buffer, 3 μL 10 mM dNTP mix, 0.5 μL 100 uM px330-F1 primer (SEQ ID No. 161 in Table 7), 0.5 μL 100 uM px330-R1 primer (SEQ ID No. 162 in Table 7), 130 μL nuclease free water, and 14 Standard Taq Polymerase. Master mix was vortexed briefly, then aliquotted 504 to 3×PCR tubes labeled 1-3. A pipette tip was dabbed into colony #1 on the agar plate and then pipetted up and down in PCR tube #1. Repeated for each colony being screened using a fresh tip for each colony. Tubes were placed in thermal cycler to run the following program: 95° C. for 5 min, 95° C. for 30 seconds, 52° C. for 30 seconds, 68° C. for 30 seconds, cycle step 2-4 for 30 cycles, 68° C. for 5 min, hold at 4° C. until use. PCR Cleanup was performed using Qiagen PCR Cleanup Kit and followed manufacturer's protocol. The product was eluted in nuclease free water.

Preparing samples for sequencing: a solution was made by mixing 120 ng PCR product, 6.4 pmols px330-F1 primer (1 μL of 6.4 μM stock), and nuclease free water that brought the final volume to 12 μL. After the sequence data was obtained, correct sequence inserts were identified. Glycerol stocks of colonies with correct inserts were prepared. On the LB agar plate labeled during colony PCR with #1-3, the correctly inserted colonies were inoculated in 5 mL LB medium with ampicillin by dabbing with a pipette tip and ejecting into the tube of medium. Liquid culture was grown out until an OD was reached between 1.0 and 1.4. 500 μL of bacterial culture was added to 500 μL of sterile 50% glycerol in a cryovial and placed immediately on dry ice until transfer to −80° C. freezer.

TABLE 6
Exemplary oligonucleotides for making guide RNA constructs
targeting GGTA1, CMAH, NLRC5, C3, GG1, and B4GALNT2

| | SEQ | | SEQ | |
|---|---|---|---|---|
| | ID | | ID | |
| Gene | No. | Forward sequence (5′ to 3′) | No. | Reverse sequence (5′ to 3′) |
| C3 | 113 | acaccgcaaggggatattcgggtttg | 114 | aaaacaaacccgaatatccccttgcg |
| c3 | 115 | acaccggcgctctttgggaacgtccg | 116 | aaaacggacgttcccaaagagcgccg |
| c3 | 117 | acaccgacgacaatggtctggcccag | 118 | aaaactgggccagaccattgtcgtcg |
| B4GALNT | 119 | acaccgtgcttttggtcctgagcgtg | 120 | aaaacacgctcaggaccaaaagcacg |
| 2 (option1) | | | | |
| B4GALNT | 121 | acaccgtcgatcctcaagatattgag | 122 | aaaactcaatatcttgaggatcgacg |
| 2 (opition2) | | | | |
| GGTA1 | 123 | acaccggggagagaagcagaggatgg | 124 | aaaaccatcctctgcttctctccccg |
| GGTA1 | 125 | acaccgctgcttgtctcaactgtaag | 126 | aaaacttacagttgagacaagcagcg |
| GGTA1 | 127 | acaccgaatacatcaacagcccagag | 128 | aaaactctgggctgttgatgtattcg |
| GGTA1 | 129 | acaccgcccagaaggttctttgttcg | 130 | aaaacgaacaaagaaccttctgggcg |
| GGTA1 | 131 | acaccgttggcagcagtgctcagagg | 132 | aaaacctctgagcactgctgccaacg |
| GGTA1 | 133 | acaccgggggccgggagccgaggtg | 134 | aaaaccacctcggctcccggcccccg |
| | | g | | |
| GGTA1 | 135 | acaccgcacccagcttctgccgatcg | 136 | aaaacgatcggcagaagctgggtgcg |
| GGTA1 | 137 | acaccgagagggggctgatcactgtg | 138 | aaaacacagtgatcagccccctctcg |
| CMAH | 139 | acaccgtagaaaaggatgaagaaaag | 140 | aaaacttttcttcatccttttctacg |
| CMAH | 141 | acaccgccaaatcttcaggagatctg | 142 | aaaacagatctcctgaagatttggcg |
| CMAH | 143 | acaccgatctgggttctgaatcccag | 144 | aaaactgggattcagaacccagatcg |
| CMAH | 145 | acaccggttctgaatcccacgggttg | 146 | aaaacaacccgtgggattcagaaccg |
| NLRC5 | 147 | acaccggcctcagaccccacacagag | 148 | aaaactctgtgtggggtctgaggccg |
| NLRC5 | 149 | acaccgtactgctgctgagcacctgg | 150 | aaaaccaggtgctcagcagcagtacg |
| NLRC5 | 151 | acaccgactgttgcagggggccccag | 152 | aaaactggggccccctgcaacagtcg |
| GG1 | 159 | gagaaaataatgaatgtcaa | 160 | ttgacattcattattttctc |

TABLE 7
Exemplary sequencing primers for px330 plasmids

| SEQ | | SEQ | |
|---|---|---|---|
| ID | Forward sequence | ID | Reverse sequence |
| No. | (5′ to 3′) | No. | (5′ to 3′) |
| 161 | gccttttgctggccat | 162 | cgggccatttaccgtaagttatg |
| | tgctc | | taacg |

### Example 2: Generating a Plasmid Expressing Guide RNA Targeting the Rosa26 Locus in Pigs

Pigs with MHC deficiencies will provide transplant grafts that induce low or no immuno-rejection in a recipient. Exogenous proteins that inhibit MHC functions will be expressed in pigs to cause MHC deficiencies. Another goal of ours further along in the project is to insert one or more exogenous polynucleotides encoding one or more proteins under the control of a ubiquitous promoter that will direct ubiquitous expression of the one or more proteins. This example show generating a plasmid expressing guide RNA targeting one of such ubiquitous promoter, Rosa26. Rosa26 promoter will direct ubiquitous expression of a gene at the Rosa26 locus. Thus transgenic pigs will be generated by inserting transgenes encoding the exogenous proteins at the Rosa26 locus, so that the gene product will be expressed in all cells in the pig. A plasmid expressing guide RNA targeting Rosa26 will be used to facilitate insertion of a transgene into the Rosa26 locus. This example shows exemplary methods for generating plasmids for targeting the Rosa26 locus in pigs using the CRISPR/cas9 system. The plasmids were generated using the px330 vector, which was be used to simultaneously express a Cas9 DNA endonuclease and a guide RNA.

Sequencing Rosa26:

For designing guide RNA targeting Rosa26 locus in a pig, Rosa26 in the pig was sequenced to provide accurate sequence information.

Primer Design: The Rosa26 reference sequence utilized to generate primers was taken from Kong et. al., Rosa26 Locus Supports Tissue-Specific Promoter Driving Transgene Expression Specifically in Pig. PLoS ONE 2014; 9(9):e107945, Li et. al., Rosa26-targeted swine models for stable gene over-expression and Cre-mediated lineage tracing. Cell Research 2014; 24(4):501-504, and Li et. al., Identification and cloning of the porcine ROSA26 promoter and its role in transgenesis. Transplantation Technology 2014:2(1). The reference sequence was then expanded by searching the pig genome database (NCBI) and by using Ensembl Genome Browser. The base sequence was separated into four 1218 base pair regions to facilitate primer design. Primers were designed using Integrated DNA Technologies' PrimerQuest Tool and then searched against the Sus scrofa reference genomic sequences using Standard Nucleotide BLAST to check for specificity. Primer length was limited to 200-250 base pairs. Primer annealing temperature was calculated using the New England Tm Calculator for a primer concentration of 1000 nM and the Taq DNA Polymerase Kit.

PCR: PCR was performed using Taq DNA Polymerase with Standard Taq Buffer (New England Biolabs). DNA template used for the PCR was extracted from cells isolated from the cloned pig. PCR conditions were 30 cycles of: 95° C., 30 seconds; 50° C., 30 seconds, 51° C. 30 seconds, 52° C. 30 seconds, 53° C. 30 seconds, 54° C. 30 seconds, 55° C. 30 seconds; and an extension step at 68° for 30 seconds. PCR products were purified using the QIAquick PCR Purification Kit (Qiagen). Primers used for sequencing are listed in Table 8.

TABLE 8
Exemplary PCR primers for sequencing Rosa26

| | Primer | |
|---|---|---|
| SEQ ID No. | Name | Sequence (from 5′ to 3′) |
| 163 | R26F008 | tctgattggctgctgaagtc |
| 164 | R26F013 | gtagccagcaagtcatgaaatc |
| 165 | R26R013 | gggagtattgctgaacctca |
| 166 | R26F014 | tcttgactaccactgcgattg |
| 167 | R26R014 | gttaggagccagtaatggagtt |
| 168 | R26F015 | agtgtctctgtctccagtatct |
| 169 | R26R015 | ttggtaaatagcaatcaactcagtg |
| 170 | R26F016 | tttctgctcaagtcacactga |
| 171 | R26R016 | caagcaatgacaacaacctgata |
| 172 | R26F017 | ttgctttctcctgatcccatag |
| 173 | R26R017 | cagtgctaatctagagcactacc |
| 174 | R26F018 | cattctcctgaagagctcagaat |
| 175 | R26R018 | tccattgggctttgtctatactt |
| 176 | R26F019 | gacaaaggaaattagcagagaacc |
| 177 | R26R019 | aactggtctttcccttggatatt |
| 178 | R26F020 | ctggctgcagcatcaatatc |
| 179 | R26R020 | gcctctattaattgcctttccc |
| 180 | R26F021 | ccattcacttcgcatccct |
| 181 | R26F005 | cgggaagtcgggagcata |
| 182 | R26R005 | gaggagaagcggccaatc |
| 183 | R26F006 | ctgctcttctcttgtcactgatt |
| 184 | R26R006 | gcgggagccactttcac |
| 185 | R26F008 | tctgattggctgctgaagtc |
| 186 | R26R008 | cgagagcaggtagagctagt |
| 187 | R26F010 | ggagtgccgcaataccttta |
| 188 | R26R010 | cctggactcatttcccatctc |
| 189 | R26F011 | gggtggagatgggaaatgag |
| 190 | R26F012 | gctacaccaccaaagtatagca |
| 191 | R26R012 | tggtggtggaacttatctgattt |
| 192 | R26F023 | agggggtacacattctcctga |
| 193 | R26R023 | gacctctgggttccattggg |
| 194 | R26F024 | caaagcccaatggaacccag |
| 195 | R26F025 | gaaggggctttcccaacagt |
| 196 | R26F026 | gcccaagacagggaaaacga |
| 197 | R26R026 | tgacaactctggtcgctctg |
| 198 | R26F028 | cagagagcctcggctaggta |
| 199 | R26R028 | aatggctccgtccgtattcc |
| 200 | R26F029 | gggaagtcgggagcatatcg |
| 201 | R26R029 | cactcccgaggctgtaactg |
| 202 | R26F030 | atggcgtgttttggttggag |
| 203 | R26R030 | ggagccactttcactgaccc |
| 204 | R26F031 | gggagggtcagtgaaagtgg |
| 205 | R26R031 | gagggccgtaccaaagacc |
| 206 | R26F032 | ggtcccaaatgagcgaaacc |
| 207 | R26R032 | gggtccgagagcaggtagag |
| 208 | R26F033 | ccgcctgaaggacgagacta |
| 209 | R26R033 | cagggcggtccttaggaaaa |
| 210 | R26F034 | gggagtgccgcaataccttt |
| 211 | R26R034 | gaaattgggctcgtcctcgt |
| 212 | R26F035 | cgaggacgagcccaatttct |
| 213 | R26R035 | agtgagggggcctaaggttt |
| 214 | R26F037 | actaccactgcgattggacc |
| 215 | R26R037 | aggagccagtaatggagttgt |
| 216 | R26F038 | cacaactccattactggctcct |
| 217 | R26R038 | ggagggtagcattccagagg |
| 218 | R26F021 | ccattcacttcgcatccct |
| 219 | R26R021 | ttgcagatgattgcttcctttc |
| 220 | R26F023 | agggggtacacattctcctga |
| 221 | R26R023 | gacctctgggttccattggg |
| 222 | R26F025 | gaaggggctttcccaacagt |
| 223 | R26R025 | gtggcgtatgccccagtatc |

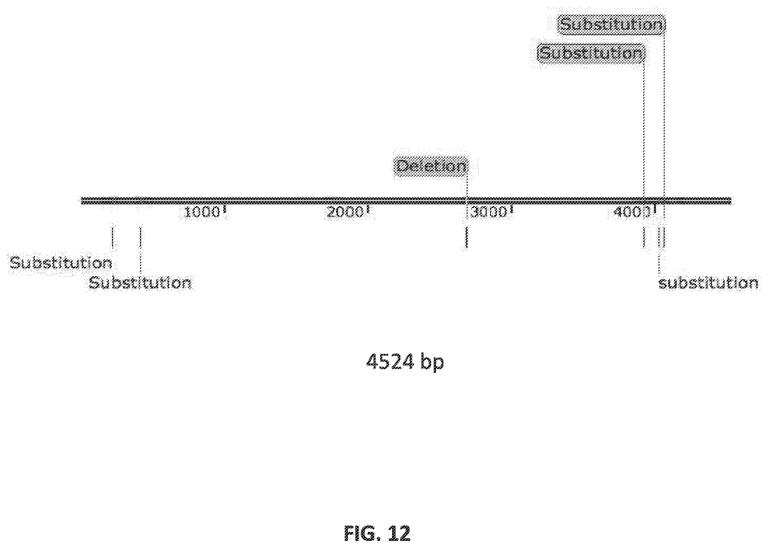

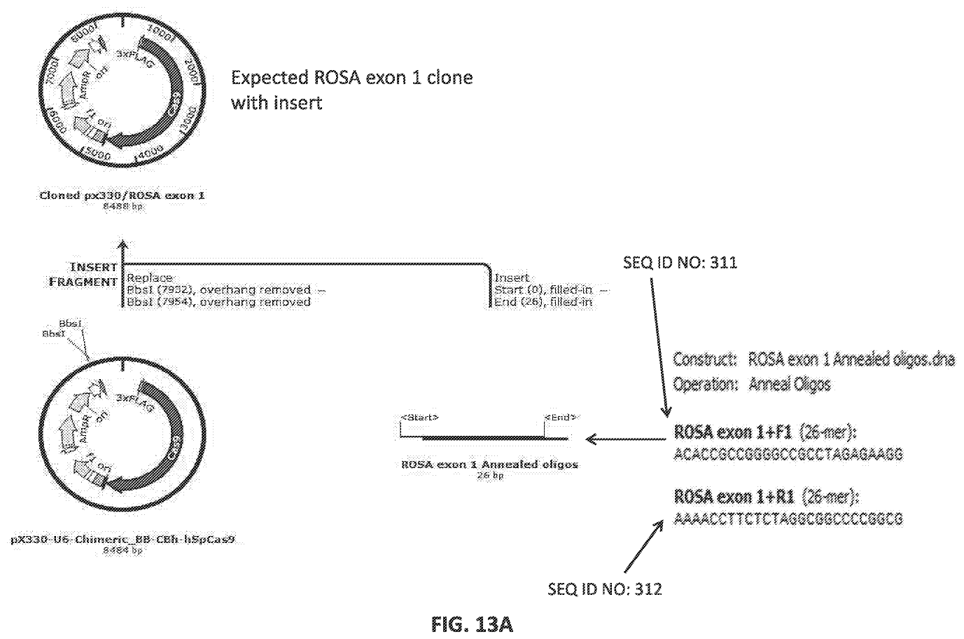

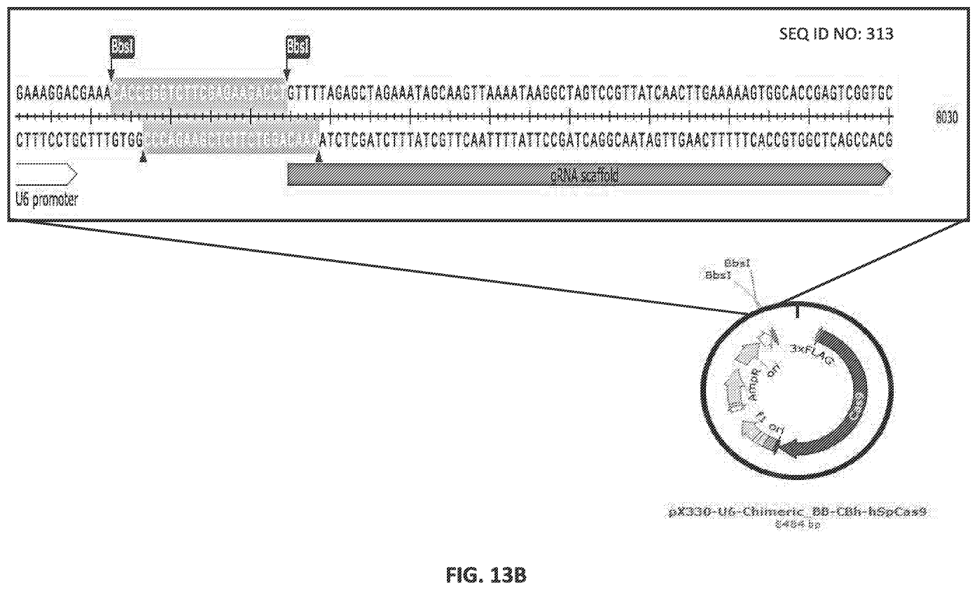

Sequencing Analysis: SnapGene software was used to align the DNA sequences. After DNA sequence results were received from the University of Minnesota Biogenomics Center, they were uploaded into the SnapGene software and aligned by the software for analysis. Base pair substitutions, deletions, and insertions were determined by referencing to the chromatograms and confirmed by comparing sequences of DNA fragments amplified using different primers. When all of the edits and confirmations were done, the resulting new DNA parent sequence was made by replacing the original parent DNA sequence with the aligned one (SEQ ID NO: 224, map shown in FIG. 12). The Rosa26 sequence was different from the reference Rosa26 sequence. For example, there were base pair substitution, at positions 223, 420, 3927, 4029, and 4066, and base pair deletion between positions 2692 and 2693. Nucleotide substitutions and deletions make this sequence unique (FIG. 12). Thus the sequencing data provided more accurate sequence information for designing guide RNA targeting the Rosa26 locus.

Generating the Plasmid Expressing Guide RNA Targeting Rosa26

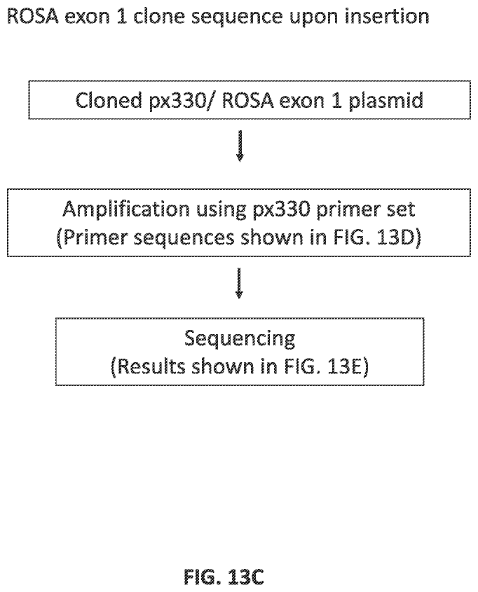

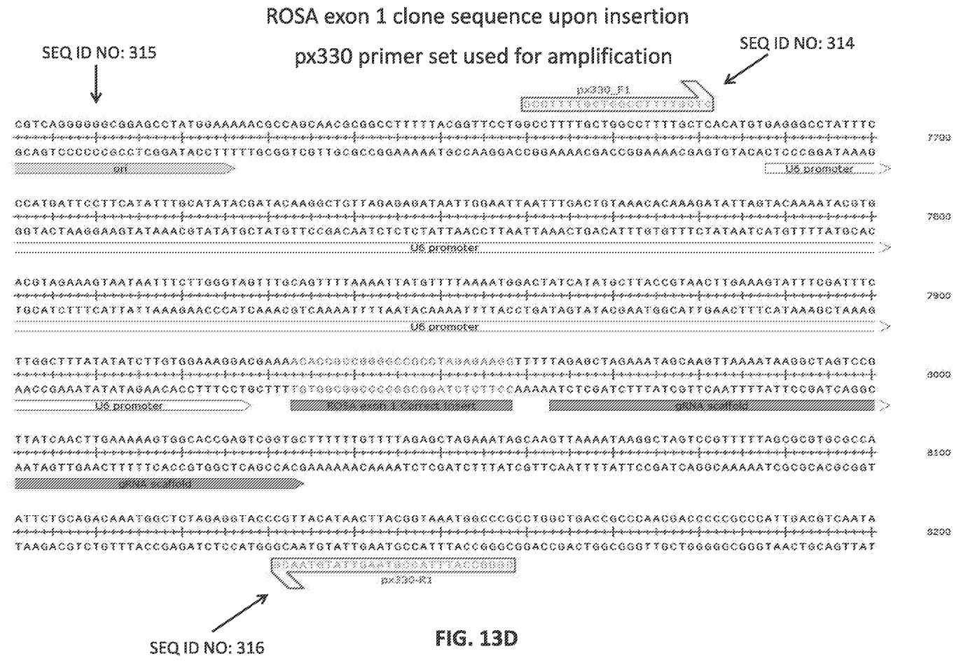

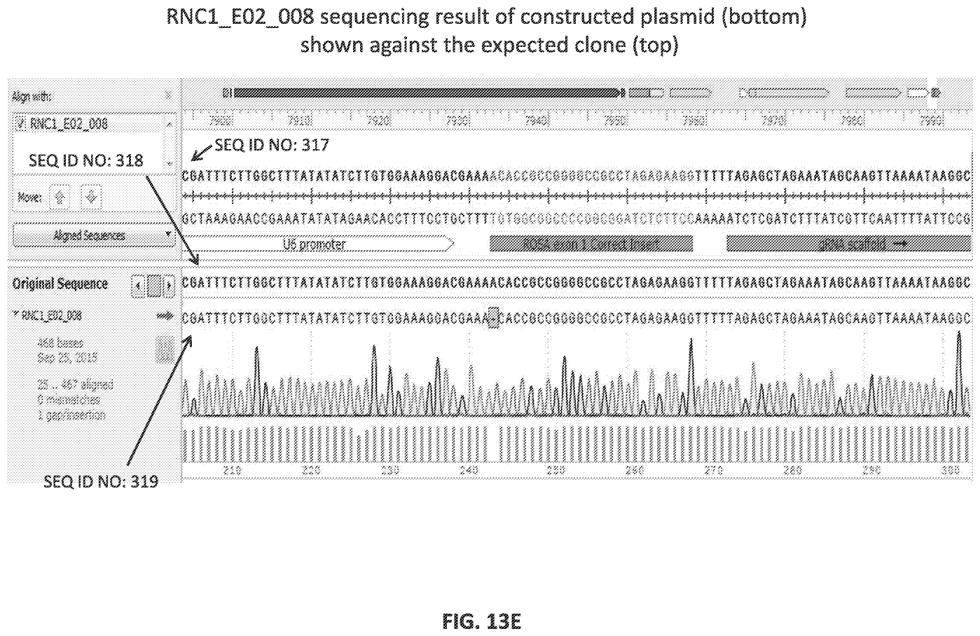

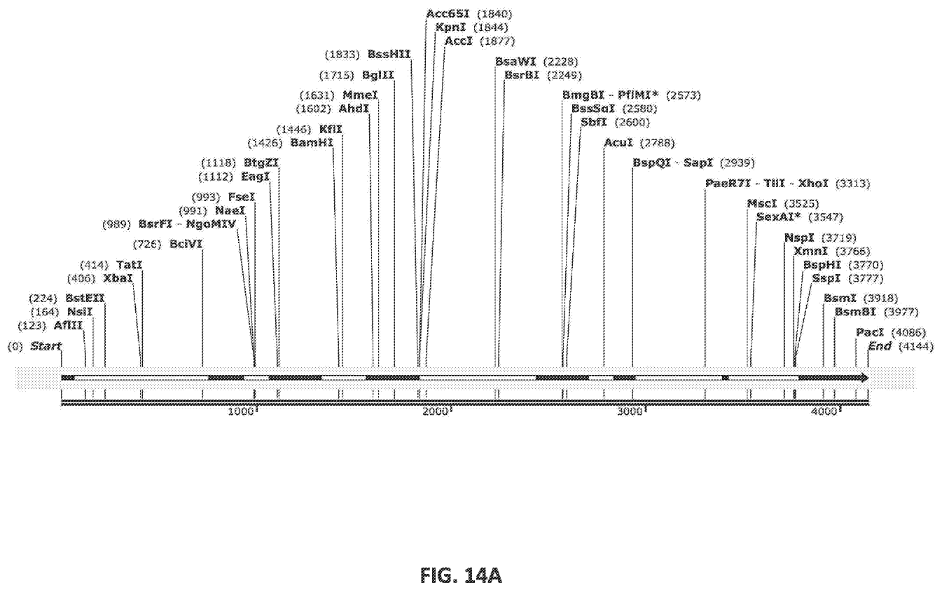

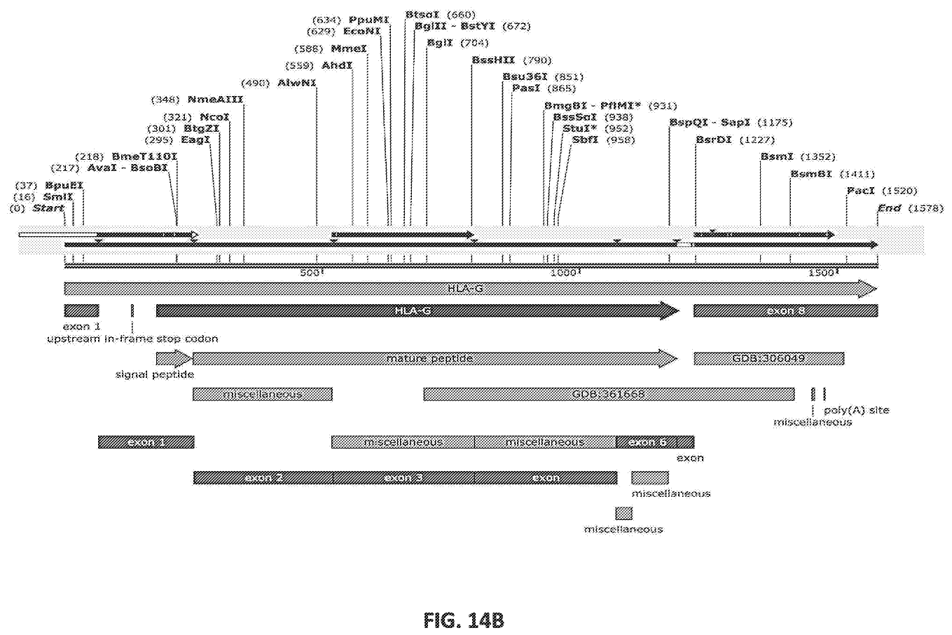

Oligonucleotides targeting Rosa26 was designed and synthesized by IDT. The sequences of the guide RNA are shown in Table 9. The px330 plasmid expressing guide RNA targeting Rosa26 was generated using methods described in Example 1. FIGS. 13A-13E show cloning strategies for cloning the plasmid targeting Rosa 26 (i.e., px330/ROSA exon 1). The constructed px330 plasmid was validated by sequencing using sequencing primers shown in Table 7.

TABLE 9
Exemplary oligonucleotides for making guide RNA constructs targeting Rosa26

| | SEQ ID | | SEQ ID | |
|---|---|---|---|---|
| Gene | No. | Forward sequence (5′ to 3′) | No. | Reverse sequence (5′ to 3′) |
| Rosa26 | 225 | acaccgccggggccgcctagagaagg | 226 | aaaaccttctctaggcggccccggcg |

### Example 3: Generating Plasmids that Simultaneously Express Two Guide RNAs

An alternative vector (e.g., px333) simultaneously expressing two guide RNAs will also be used for expressing guide RNA targeting two regions of a single gene. Targeting two regions of a single gene by CRISPR/Cas9 system will result in removal of the entire gene between the two cut sites when the DNA is repaired back together. Targeting two regions will increase the chance of producing a biallelic knockout, resulting in better sorts, more biallelic deletions, and overall a higher chance to produce pigs with a negative genotype, comparing to only targeting one locus in the gene.

The oligonucleotide pairs used in the px333 plasmid construction will contain higher G content, lower A content, and as many GGGG quadraplexes as possible, compared with the oligonucleotides used for the px330 plasmid. The GGTA1 targets will span nearly the entire GGTA1 gene, which will remove the entire gene from the genome. Furthermore, targeting multiple sites with this strategy will be used when inserting transgenes, which is another goal of ours further along in the project.

### Example 4: Isolating, Culturing and Transfecting Porcine Fetal Fibroblasts for Making Genetically Modified Pigs

To generate genetically modified pigs using a px330 plasmid expressing guide RNA targeting a gene, the px330 plasmid was transfected into porcine fetal fibroblasts. The transfected fibroblasts will express the guide RNA that causes disruption of one or more target genes. The resulting fibroblasts were used for making genetically modified pigs, e.g., by somatic cell nuclear transfer. This example shows isolation and culturing porcine fetal fibroblasts, and transfection of the fibroblasts with a px330 plasmid.

Cell Culture

Fetal fibroblasts cell lines used in the generation of genetically modified pigs included: Karoline Fetal (derived from female porcine ponor P1101, which provided a high islet yield after islet isolation), Marie Louise Fetal (derived from female porcine donor P1102, which provided a high islet yield after islet isolation), Slaughterhouse pig #41 (Male; showed a high number of islets in the native pancreas (as assessed by a very high dithizone (DTZ) score)), Slaughterhouse pig #53 (showed a high number of islets in the native pancreas as assessed by a high dithizone (DTZ) score).

Muscle and skin tissue samples taken from each of these pigs were dissected and cultured to grow out the fibroblast cells. The cells were then harvested and used for somatic cell nuclear transfer (SCNT) to produce clones. Multiple fetuses (up to 8) were harvested on day 30. Fetuses were separately dissected and plated on 150 mm dishes to grow out the fetal fibroblast cells. Throughout culture, fetus cell lines were kept separate and labeled with the fetus number on each tube or culture vessel. When confluent, cells were harvested and frozen at about 1 million cells/mL in FBS with 10% DMSO for liquid nitrogen cryo-storage.

Culture medium preparation: 5 mL Glutamax, 5 mL pen/strep, and 25 mL HI-FBS (for standard 5% FBS medium; use 10% FBS for sorted cells) were added to a 500 mL bottle of DMEM, high glucose, no glutamine, no phenol red. Centrifuge settings for spinning down all fetal fibroblasts were 5 minutes at 0.4 rcf (1600 rpm) at 4° C. Cells were thawed from liquid nitrogen storage by warming quickly to 37° C. in water bath. The thawed cells were quickly transferred to about 25 mL fresh, pre-warmed culture medium (enough to dilute the DMSO sufficiently). The cells were then spun down, the supernatant was removed and the cells were re-suspended in 1-5 mL fresh culture medium for counting or plating. Cells received a medium change every 3-4 days with pre-warmed medium, and were passaged when 90-100% confluent using TrypLE Express Dissociation Reagent.

Harvesting Adherent Fibroblasts: The medium was aspirated off the cells. DPBS was added to wash the cells. Pre-warmed (37° C.) TrypLE Express reagent was added to the cells. Minimum amount of the reagent was used to cover the cell layer thinly. The cells were incubated at 37° C. for 10 minutes. A volume of culture medium containing FBS was added to the TrypLE cell suspension to neutralize the enzyme. The cell suspension was pipetted up and down to dislodge all cells from the culture surface. The cell suspension was transferred to a 15 or 50 mL conical tube on ice. The plate/flask was checked under a microscope to ensure all cells were collected. Sometimes a medium wash helped collect cells that were left behind. The cells were spun down, and then re-suspended with fresh culture medium (between 1-5 mL for counting). If counting, a 1:5 dilution of the cells suspension was prepared by adding 20 μL cell suspension to 80 μL 0.2% Trypan Blue. The suspension was mixed well by pipetting up and down. 12-14 μL of the dilution was added to a hemocytometer to count the 4 corners. The numbers were averaged. For example, counting 20, 24, 22, 22 for each corner yielded an average of 22. This number was multiplied by the dilution factor 5, yielding 110×10⁴ cells/mL. The number was adjusted to 10⁶ by moving the decimal two places to the left, 1.10×10⁶ cells/mL. Finally, the numbers were multiplied by how many mL's the original sample was taken from to get the total number of cells.

Transfection of Fetal Fibroblasts

This experiment was to transfect fetal fibroblasts. The transfected fetal fibroblasts were used to generate genetically modified animal using the somatic cell nuclear transfer technique.

The GFP plasmid used (pSpCas9(BB)-2A-GFP) for transfection was an exact copy of the px330 plasmid, except that it contained a GFP expression region.

GFP transfected control cells: Transfections were done using the Neon Transfection System from Invitrogen. Kits came in 10 μL and 100 μL tip sizes. A day or two before the experiment, cells were plated in appropriate culture vessel depending on size of experiment and desired cell number and density. About 80% confluence was achieved on day of transfection.

On the day of the experiment, Neon module and pipette stand was set up in a biohood. A Neon tube was placed in the pipette stand and 3 mL of Buffer E (Neon Kit) was added to the Neon tube. The module was turned on and adjusted to desired settings (for fetal porcine fibroblasts: 1300 V, 30 ms, 1 pulse). Cells were harvested using TrypLE and counted to determine the experimental setup. Needed amount of cells were transferred to a new tube and remaining cells were re-plated. Cells were spun down after counting, and re-suspended in PBS to wash. The cells were spun down and re-suspended in Buffer R (Neon Kit) according to Table 10 for the number of cells and tip sizes.

TABLE 10
Exemplary Neon ® plate formats, volumes, and recommended kits

| | | DNA | siRNA | | | Vol. plating | | | Buffer R or | |
|---|---|---|---|---|---|---|---|---|---|---|
| Format | Cell Type | (μg) | (nM) | Neon ®Tip | | medium | | Cell no. | Buffer T | |
| 96-well | Adherent | 0.25-0.5 | 10-200 | 10 | μL | 100 | μL | 1-2 × 10⁴ | 10 | μL/well |
| | Suspension | 0.5-1 | | 10 | μL | | | 2-5 × 10⁴ | 10 | μL/well |
| 48-well | Adherent | 0.25-1 | 10-200 | 10 | μL | 250 | μL | 2.5-5 × 10⁴ | 10 | μL/well |
| | Suspension | 0.5-2 | | 10 | μL | | | 5-12.5 × 10⁴ | 10 | μL/well |
| 24-well | Adherent | 0.5-2 | 10-200 | 10 | μL | 500 | μL | 0.5-1 × 10⁵ | 10 | μL/well |
| | Suspension | 0.5-3 | | 10 | μL | | | 1-2.5 × 10⁵ | 10 | μL/well |
| 12-well | Adherent | 0.5-3 | 10-200 | 10 | μL | 1 | mL | 1-2 × 10⁵ | 10 | μL/well |
| | Suspension | 0.5-3 | | 10 | μL | | | 2-5 × 10⁵ | 10 | μL/well |
| 6-well | Adherent | 0.5-3 | 10-200 | 10 μL/100 μL | | 2 | mL | 2-4 × 10⁵ | 10 μL or | |
| | | (10 μL) | | | | | | | 100 μL/well | |
| | | 5-30 | | | | | | | | |
| | | (100 μL) | | | | | | | | |
| | Suspension | 0.5-3 | | 10 μL/100 μL | | | | 0.4-1 × 10⁶ | 10 μL or | |
| | | (10 μL) | | | | | | | 100 μL/well | |
| | | 5-30 | | | | | | | | |
| | | (100 μL) | | | | | | | | |
| 60 mm | Adherent | 5-30 | 10-200 | 100 | μL | 5 | mL | 0.5-1 × 10⁶ | 100 | μL/well |
| | Suspension | 5-30 | | 100 | μL | | | 1-2.5 × 10⁶ | 100 | μL/well |
| 10 cm | Adherent | 5-30 | 10-200 | 100 | μL | 10 | mL | 1-2 × 10⁶ | 100 | μL/well |

| | | | | | | |
|---|---|---|---|---|---|---|
| Suspension | 5-30 | 100 | μL | 2-5 × 10⁶ | 100 | μL/well |

Appropriate amount of DNA according to Table 10 was added to cell suspension and mixed by pipetting up and down. A Neon tip was applied from the kit to the Neon pipette to aspirate the volume of cell suspension into the Neon tip. The pipette was placed into the Neon tube in the pipette stand so that the Neon tip was submerged in the Buffer E. START was pressed on module interface until a “complete” message appeared. The pipette was removed from the pipette stand to eject the cell suspension into a volume of pre-warmed culture medium without antibiotics in a well of appropriate size according to Table 10.

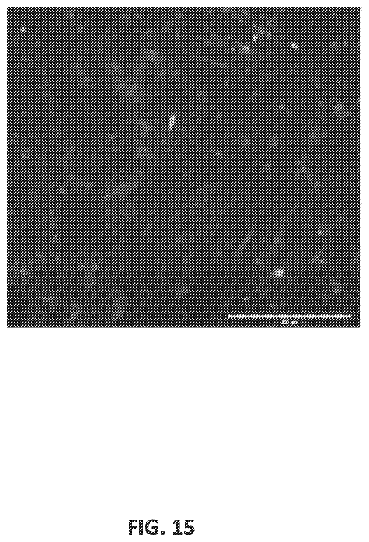

The above steps were repeated until the entire cell suspension was used. Neon tips were changed every 2 transfections, and Neon tubes were changed every 10 transfections. The cells were incubated at 37° C. for 24 hours, and then the medium was changed with normal culture medium containing antibiotics. The resulting cells were cultured for about 5 days to allow for Cas9 cleavage, complete recycling of surface proteins after gene knockout, and proper cell division before sorting. The cells transfected with the GFP plasmid were shown in FIG. 15.

### Example 5: Fluorescence In Situ Hybridization (FISH) to the GGTA1 Gene

Gene disruption by CRISPR/cas9 was verified using FISH in a cell. This example shows exemplary methods for detecting GGTA1 gene using fluorescence in situ hybridization (FISH). The methods here were used to verify the presence or absence of a GGTA1 gene in a cell from an animal (e.g., an animal with GGTA1 knocked out).

Preparation of FISH Probes:

GGTA1 DNA was extracted from an RP-44 pig BAC clone (RP44-324B21) using an Invitrogen PureLink kit. The DNA was labeled by nick translation reaction (Nick Translation Kit—Abbott Molecular) using Orange—552 dUTP (Enzo Life Science). Sizes of the nick translated fragments were checked by electrophoresis on a 1% TBE gel. The labeled DNA was precipitated in COT-1 DNA, salmon sperm DNA, sodium acetate and 95% ethanol, then dried and re-suspended in 50% formamide hybridization buffer.

Hybridization of FISH Probes:

The probe/hybridization buffer mix and cytogenetic slides from pig fibroblasts (15AS27) were denatured. The probe was applied to the slides, and the slides were hybridized for 24 hours at 37° C. in a humidified chamber.

FISH Detection, Visualization and Image Capture:

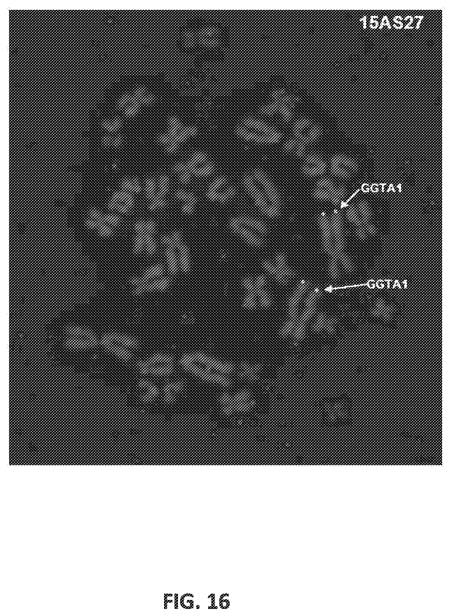

After hybridization, the FISH slides were washed in a 2×SSC solution at 72° C. for 15 seconds, and counterstained with DAPI stain. Fluorescent signals were visualized on an Olympus BX61 microscope workstation (Applied Spectral Imaging, Vista, Calif.) with DAPI and FITC filter sets. FISH images were captured using an interferometer-based CCD cooled camera (ASI) and FISHView ASI software. The FISH image is shown in FIG. 16.

### Example 6: Phenotypic Selection of Cells with Cas9/Guide RNA-Mediated GGTA1 Knockout

Disruption of GGTA1 gene by the Cas9/guide RNA system were verified by labeling GGTA1 gene products. The GGTA1 knockout will be used as a marker for phenotypic sorting in knockout experiments. The GGTA1 gene encoded for a glycoprotein found on the surface of pig cells that if had been knocked out, would result in the glycoprotein being absent on the cell's surface. The lectin used to sort for GGTA1 negative cells was Isolectin GS-IB₄ Biotin-XX conjugate, which selectively bound terminal alpha-D-galactosyl residues, such as the glycoprotein produced by the GGTA1 gene.

Porcine fetal fibroblast cells were transfected with px330 plasmid expressing guide RNA targeting GGTA1 (generated in Example 1).

To select for negative cell after transfection, the cells were allowed to grow for about 5 days to recycle their surface proteins. The cells were then harvested, and labeled with the IB₄ lectin. The cells were then coated with DynaBeads Biotin-Binder, which were 2.8 micron supermagnetic beads that had a streptavidin tail that bound very tightly with the biotin-conjugated lectin on the surface of the cells. When placed in a magnet, the “positive” cells with lectin/beads bound on the surface stick to the sides of the tube, while the “negative” cells did not bind any beads and remained floating in suspension for an easy separation.

In detail, the cells were harvested from a plate using a TrypLE protocol and collected into a single tube. The cells were spun down, and re-suspended in 1 mL of sorting medium (DMEM, no supplements) to count. If less than 10 million cells, the cells were spun down and the supernatant was discarded. In a separate tube, IB₄ lectin (1 μg/μL) was diluted by 5 μL to 1 mL of sorting medium (final concentration 5 μg/mL). The cell pellet was re-suspended with the 1 mL of diluted lectin. The cell suspension was incubated on ice for about 15-20 minutes, with gentle sloshing every few minutes.

Biotin beads were prepared during incubation. A bottle of beads were vortexed for 30 seconds. 20 μL beads/1M cells were added to 5 mL of sorting medium in a 15 mL conical tube. The tube was vortexed, placed in DynaMag-15 magnet and let stand for 3 minutes. Medium were removed. 1 mL of fresh sorting medium was added and the tube was vortexed to wash the beads. The washed beads were placed on ice until use.

After cell incubation, cell suspension's volume was brought to 15 mL with sorting medium to dilute the lectin. The cells were spun and re-suspended with 1 mL of the washed biotin beads. The suspension was incubated on ice for 30 minutes in a shaking incubator at 125 rpm. The cell suspension was removed from shaking incubator and inspected. Small aggregates might be observed.

5 mL of sorting medium was added to the cell suspension and the tube was placed in the DynaMag-15 for 3 minutes. The first fraction of “negatives” cells was collected and transferred to a new 15 mL conical tube. Another 5 mL sorting medium was added to wash the “positive” tube that was still on the magnet. The magnet was inverted several times to mix the cell suspension again. The tube was let stand for 3 minutes to separate cells. The second “negative” fraction was then removed and combined with the first fraction. 10 mL sorting medium was added to the “positive” tube. The tube was removed from the magnet, and placed in an ice bath until ready to use.

The tube of “negative” fractions was placed onto the magnet to provide a secondary separation and remove any bead-bound cells that might have crossed over from the first tube. The tube was kept on the magnet for 3 minutes. The cells were pipetted away from the magnet and transferred into a new 15 mL conical tube. The original “positive” tube and the double sorted “negative” tube were balanced and cells in them were spun down. The pellet of the “positives” appeared a dark, rusty red. The “negative” pellet was not visible, or appeared white.

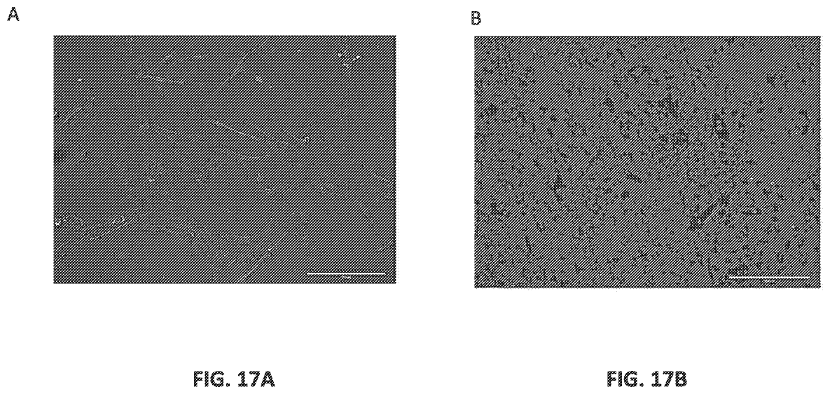

Each pellet was re-suspended in 1 mL of fresh culture medium (10% FBS) and plated into separate wells on a 24-well plate. The wells were inspected under a microscope and diluted to more wells if necessary. The cells were cultured at 37° C. The genetically modified cells, i.e., unlabeled cells were negatively selected by the magnet (FIG. 17A). The non-genetically modified cells, i.e., the labeled cells had accumulated ferrous beads on the cell surface (FIG. 17B).

### Example 7. Generation and Characterization of GGTA1/NLRC5 Knockout Pigs

This example shows exemplary methods for generating knockout pigs. A knockout pig can have reduced protein expression of two or more of the following: NLRC5, TAP1, C3, CXCL10, MICA, MICB, CIITA, CMAH, GGTA1 and/or B4GALNT2. One of such knockout pig was a GGTA1/CMAH/NLRC5 knockout pig using CRISPR/cas9 system. The pigs provided islets for transplantation. Porcine islets with disrupted GGTA1/CMAH/NLRC5 had MHC class I deficiency and will induce low or no immuno-rejection when transplanted to a recipient.

Transfection of Fetal Fibroblasts

The px330 plasmids expressing guide RNA targeting GGTA1, CMAH, and NLRC5 generated in Example 1 were transected in porcine fetal fibroblasts. Pig fetal fibroblasts were cultured in DMEM containing 5-10% serum, glutamine and penicillin/streptomycin. The fibroblasts were co-transfected with two or three plasmids expressing Cas9 and sgRNA targeting the GGTA1, CMAH or NLRC5 genes using Lipofectamine 3000 system (Life Technologies, Grand Island, N.Y.) according to the manufacturer's instructions.

Counter-Selection of GGTA1 KO Cells

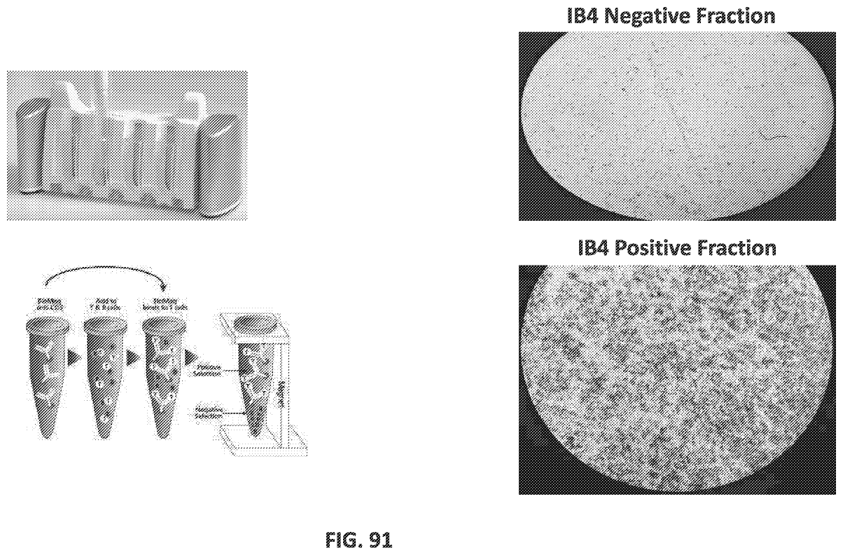

Four days after transfection, the transfected cells were harvested and labeled with isolectin B4 (IB4)-biotin. Cells expressing αGal were labeled with biotin conjugated IB4 and depleted by streptavidin coated Dynabeads (Life Technologies) in a magnetic field (FIG. 91). The αGal deficient cells were selected from the supernatant. The cells were examined by microscopy. The cells containing no or very few bound beads after sorting were identified as negative cells.

DNA Sequencing Analysis of the CRISPR/Cas9 Targeted GGTA1 and NLRC5 Genes

Genomic DNA from the IB4 counter-selected cells and cloned pig fetuses were extracted using Qiagen DNeasy Miniprep Kit. PCR was performed with GGTA1 and NLRC5 specific primer pairs as shown in Table 11. DNA polymerase, dNTPack (New England Biolabs) was used and PCR conditions for GGTA1 were based on annealing and melting temperature ideal for those primers. The PCR products were separated on 1% agarose gel, purified by Qiagen Gel Extraction Kit and sequenced by the Sanger method (DNA Sequencing Core Facility, University of Minnesota) with the specific sequencing primers as shown in Table 7.

TABLE 11
Exemplary PCR primers for amplifying genomic DNA from genetically modified
cells and animals

| | SEQ ID | Forward sequence (5′ to | SEQ | |
|---|---|---|---|---|
| Gene | No. | 3′) | ID No. | Reverse sequence (5′ to 3′) |
| GGTA1 | 227 | cttcgtgaaaccgctgtttatt | 228 | gactggaggactttgtatat |
| CMAH | 229 | tgagttccttacgtggaatgtg | 230 | tcttcaggagatctgggttct |
| NLRC5 | 231 | ctgctctgcaaacactcaga | 232 | tcagcagcagtacctcca |

Somatic Cell Nuclear Transfer (SCNT)

SCNT was performed as described by Whitworth et al. Biology of Reproduction 91(3):78, 1-13, (2014). The SCNT was performed using in vitro matured oocytes (DeSoto Biosciences Inc., St. Seymour, Tenn.). Cumulus cells were removed from the oocytes by pipetting in 0.1% hyaluronidase. Only oocytes with normal morphology and a visible polar body were selected for SCNT. Oocytes were incubated in manipulation media (Ca-free NCSU-23 with 5% FBS) containing 5 μg/mL bisbenzimide and 7.5 μg/mL cytochalasin B for 15 min. Oocytes were enucleated by removing the first polar body plus metaphase II plate. A single cell was injected into each enucleated oocyte, fused, and activated simultaneously by two DC pulses of 180 V for 50 μsec (BTX cell electroporator, Harvard Apparatus, Hollison, Mass., USA) in 280 mM Mannitol, 0.1 mM CaCl₂, and 0.05 mM MgCl₂. Activated embryos were placed back in NCSU-23 medium with 0.4% bovine serum albumin (BSA) and cultured at 38.5° C., 5% CO₂ in a humidified atmosphere for less than 1 hour, and transferred into the surrogate pigs.

Producing Genetically Modified Pigs Using Embryos

Embryos for transferring to the surrogate pigs were added to a petri dish filled with embryo transferring media. A 0.25 ml sterile straw for cell cryopreservation was also used. Aspiration of embryos was performed at 25-35° C.

Aspiration of embryos was performed following this order: media layer-air layer-media layer-air layer-embryo layer-air layer-media layer-air layer-media layer. When the straw sterilized with EO gas was used, its interior was washed by repeating aspiration and dispensing of the medium for embryo transplantation 1-3 times, before aspiration of embryos. After the aspiration, the top end of straw was sealed by a plastic cap. To keep the aspirated and sealed straw sterile, a plastic pipette (Falcon, 2 ml) was cut in a slightly larger size than the straw, put therein, and sealed with a paraffin film. The temperature of the sealed straw was maintained using a portable incubator, until shortly before use.

Embryos and estrus-synchronized surrogate mothers were prepared. Transferring of embryos will be performed by exposing ovary through laparotomy of the surrogate mothers. After anesthetization, the mid-line of the abdominal region was incised to expose the uterus, ovary, oviduct, and fimbriae. The straw aspirating embryos were aseptically taken from the portable incubator, and inserted into the inlet of oviduct. The inserted straw was moved up to the ampullary-isthmic junction region. After the insertion procedure, the straw was cut at the air containing layer on the opposite using scissors. A 1 cc syringe was mounted on the cut end, and approximately 0.3 cc of air was injected to release the embryos and medium from the straw into the oviduct. At this time, 5 mm of the top end of a 0.2 ml yellow tip was cut off and used to connect the syringe and straw.

After the embryo transfer, the exposed uterus, ovary, oviduct, and fimbriae were put in the abdominal cavity, and the abdominal fascia was closed using an absorbable suture material. Then, the surgical site was cleaned with Betadine, and treated with antibiotics and anti-inflammatory and analgesic drugs. A pregnancy test of the surrogate mother transplanted with embryos was performed, followed by induction of delivery of non-human animals that successfully got pregnant.

Pregnancy and Fetuses

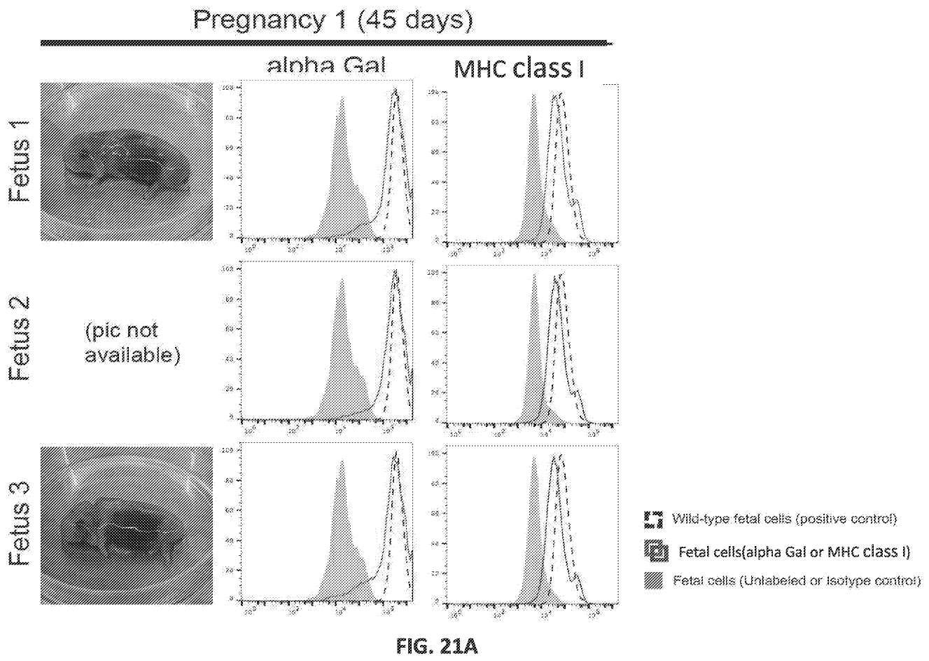

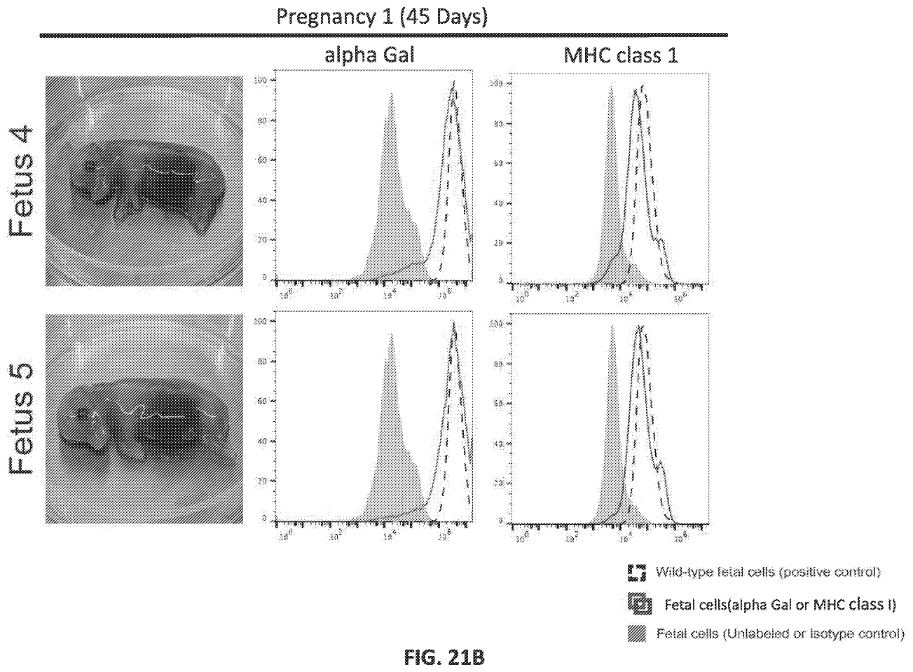

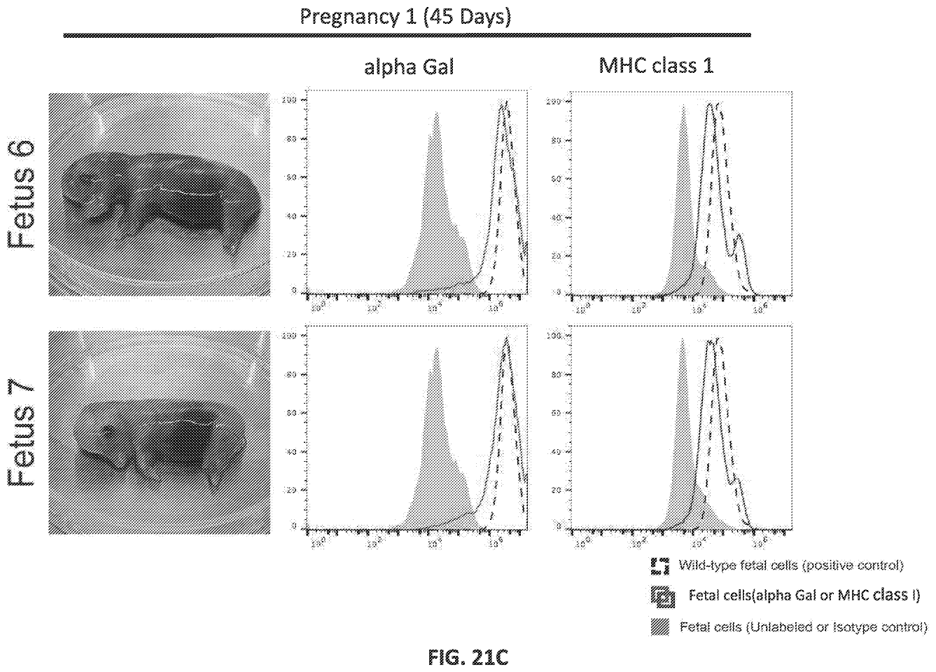

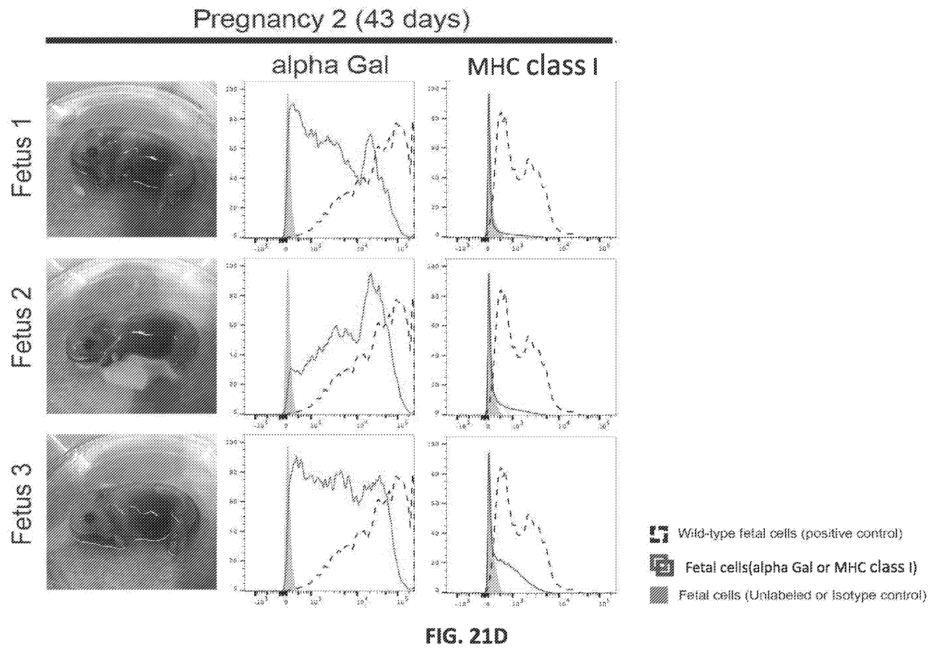

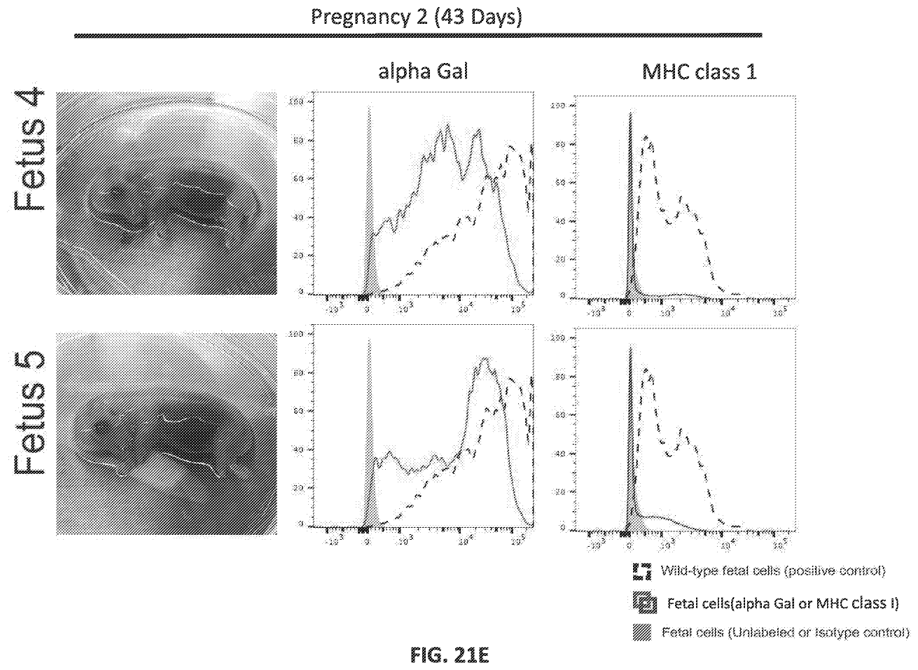

Two litters of pig fetuses (7 from pregnancy 1 and 5 from pregnancy 2) were obtained. Fetuses were harvested at day 45 (pregnancy 1) or 43 (pregnancy 2) and processed for DNA and culture cell isolation. Tissue fragments and cells were plated in culture media for 2 days to allow fetal cells to adhere and grow. Wild type cells (fetal cells not genetically modified) and fetal cells from pregnancy 1 or 2 were removed from culture plates and labeled with IB4 lectin conjugated to alexa fluor 488 or anti-porcine MHC class I antibody conjugated to FITC. Flow cytometric analysis was performed and data shown in FIG. 21A-C: Pregnancy 1 or FIG. 21D-E: Pregnancy 2. The histogram for the WT cells is included in each panel to highlight the decrease in overall intensity of each group of fetal cells. Of specific interested is the decrease in alpha Gal and MHC class I labeling in pregnancy 1 indicated as a decrease in peak intensity. In pregnancy 2, fetus 1 and 3 have a large decrease in alpha gal labeling and significant reduction in MHC class 1 labeling as compared to WT fetal cells.

Genotypes of the Fetuses

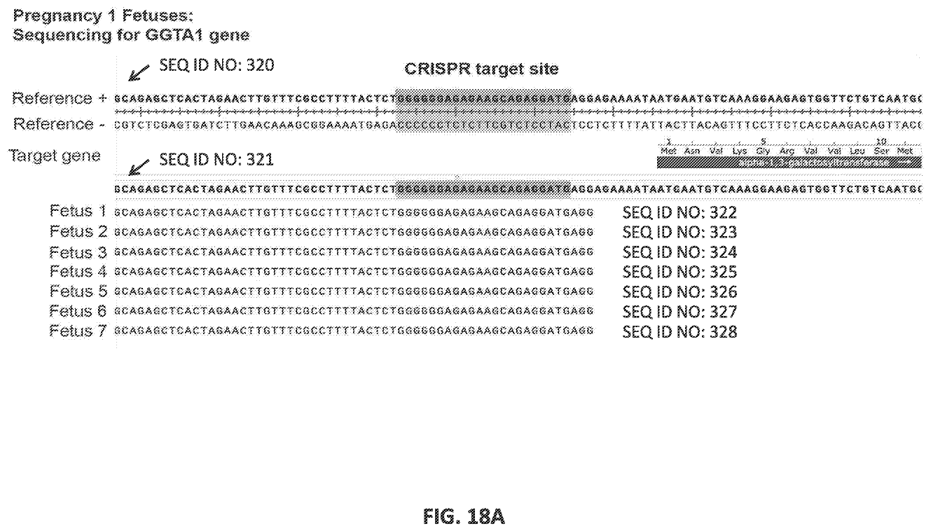

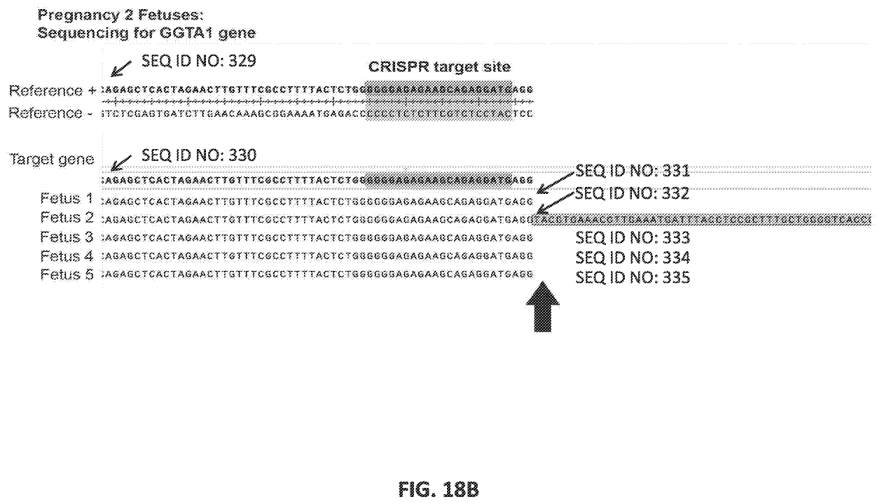

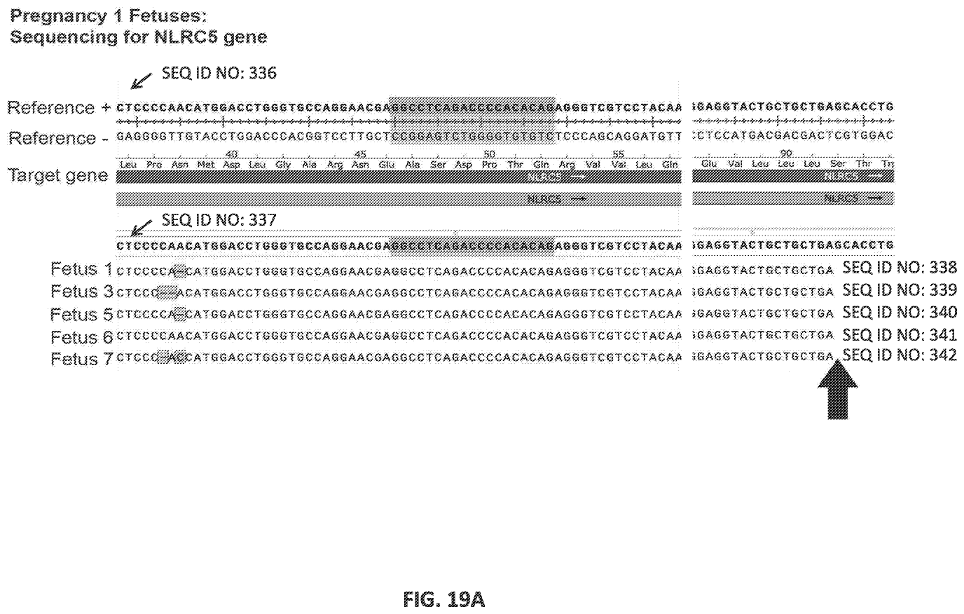

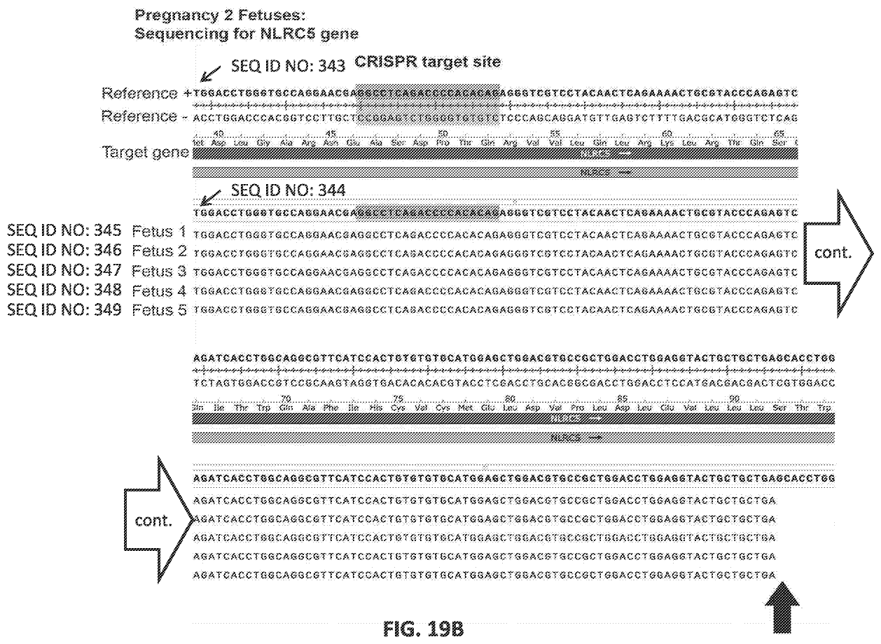

DNA from fetal cells was subjected to PCR amplification of the GGTA1 (compared to Sus scrofa breed mixed chromosome 1, Sscrofa10.2 NCBI Reference Sequence: NC_010443.4) or NLRC5 (consensus sequence) target regions and the resulting amplicons were separated on 1% agarose gels (FIGS. 18A, 18B, 19A, and 19B). Amplicons were also analyzed by sanger sequencing using the forward primer alone from each reaction. The results are shown as Pregnancy 1 fetuses 1, 2, 4, 5, 6, and 7 truncated 6 nucleotides after the target site for GGTA1. Fetus 3 was truncated 17 nucleotides after the cut site followed by a 2,511 (668-3179) nucleotide deletion followed by a single base substitution. Truncation, deletion and substitution from a single sequencing experiment containing the alleles from both copies of the target gene can only suggest a gene modification has occurred but not reveal the exact sequence for each allele. From this analysis it appears that all 7 fetuses contained a single allele modification. Sequence analysis of the NLRC5 target site for fetuses from pregnancy 1 was unable to show consistent alignment suggesting an unknown complication in the sequencing reaction or varying DNA modifications between NLRC5 alleles that complicate the sanger sequencing reaction and analysis. Pregnancy 2 fetal DNA samples 1, 3, 4, and 5 were truncated 3 nucleotides from the GGTA1 gene target site. Fetus 2 had variability in sanger sequencing that suggests a complex variability in DNA mutations or poor sample quality. However, fetal DNA template quality was sufficient for the generation of the GGTA1 gene screening experiment described above. NLRC5 gene amplicons were all truncated 120 nucleotides downstream of the NLRC5 gene cut site.

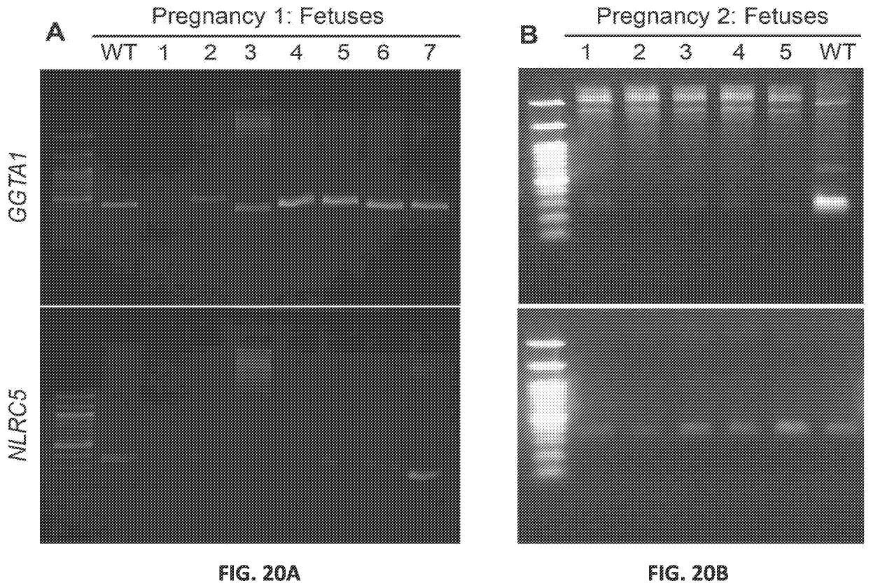

Fetal DNA (from wild type (WT) controls, and fetuses 1-7 from pregnancy 1) was isolated from hind limb biopsies and the target genes NLRC5 and GGTA were amplified by PCR. PCR products were separated on 1% agarose gels and visualized by fluorescent DNA stain. The amplicon bands in the WT lane represent unmodified DNA sequence. An increase or decrease in size of an amplicon suggested an insertion or deletion within the amplicon, respectively. Variations in the DNA modification between alleles in one sample might make the band appear more diffuse. Minor variations in the DNA modification were possible to resolve by a 1% agarose gel. The results are shown in FIGS. 20A-20B. A lack of band as in the NLRC5 gel (fetuses 1, 3, and 4 of Pregnancy 1; FIG. 20A bottom) suggested that the modification to the target regions was disrupted the binding of DNA amplification primers. The presence of all bands in GGTA1 targeting experiment suggests that DNA quality was sufficient to generate DNA amplicons in the NLRC5 targeting PCR reactions. Fetuses 1, 2, 4, and 5 of Pregnancy 1 (FIG. 20A, top) had larger GGTA1 amplicons, suggesting an insertion within the targeted area. For fetus 3 of Pregnancy 1 (FIG. 20A, top), the GGTA1 amplicon migrated faster than the WT control, suggesting a deletion within the targeted area. For fetuses 6 and 7 of Pregnancy 1 (FIG. 20A, bottom), the NLRC5 amplicons migrated faster than the WT, suggesting a deletion with in the target area. Fetuses 1-5 of Pregnancy 2, (FIG. 20B, top) GGTA1 amplicons were difficult to interpret by size and were diffuse as compared to the WT control. Fetuses 1-5 (FIG. 20B, bottom) NLRC5 amplicons were uniform in size and density as compared to the wild type control.

Given the variation in phenotypic results for the alpha Gal and MHC class 1 flow cytometric labeling there is considerable variation in the bi-allelic mutations in the GGTA1 and NLRC5 genes. This observation is supported by differences in band size in the agarose gels, truncated gene products, and sequencing challenges (FIGS. 18A-18B, 19A-19B, 20A-20B, and 21A-21E). Cloning of individual alleles will be performed to fully decipher the sequence modifications. However, the phenotypic, DNA sequencing, and functional analysis of fetuses support the creation of biallelic GGTA1 and NLRC5 gene modifications in fetal pigs.

Impact of Gene Knockout on Proliferation of Human Immune Cells

Next, with cells from fetus 3 of pregnancy 1, co-culture assays were performed to evaluate the impact of decreased MHC class I expression on proliferation of human immune cells.

Mixed Lymphocyte Reaction (MLR)

Co-cultures were carried out in flat-bottom, 96-well plates. Human PBMCs labeled with Carboxyfluorescein succinimidyl ester (CFSE) (2.5 μM/ml), were used as responders at 0.3-0.9×10⁵ cells/well. Wild type or Porcine fibroblasts at 0.1-0.3×10⁵ cells/well (from wild type pigs or the GGTA1/NLRC5 knockout fetuses) were used as stimulators at stimulator-responder ratios of 1:1, 1:5 and 1:10. MLR co-cultures were carried out for 4 days in all MLR assays. In another parallel experiment, total PBMCs cells were stimulated with phytohaemagglutinin (PHA) (2 ug/ml) as positive control.

Cultured cells were washed and stained with anti-CD3 antibody, anti-CD4 antibody and anti-CD8 antibody followed by formaldehyde fixation and washed. BD FACS Canto II flow cytometer was used to assess the proliferative capacity of CD8+ and CD4+ T cells in response to fibroblasts from the GGTA1/NLRC5 knockout fetus compared to unmodified porcine fibroblast cells. Data were analyzed using FACS diva/Flow Jo software (Tri star, San Diego, Calif., USA), and percentage CFSE dim/low was determined on pre gated CD8 T cells and CD4 T cells.

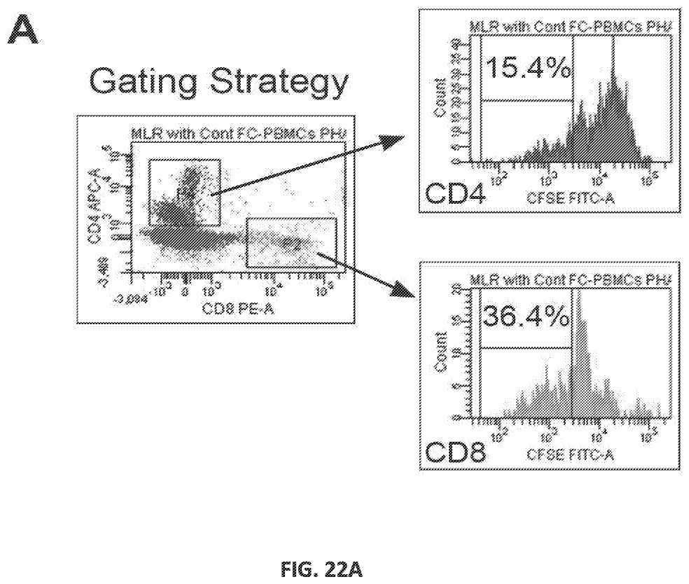

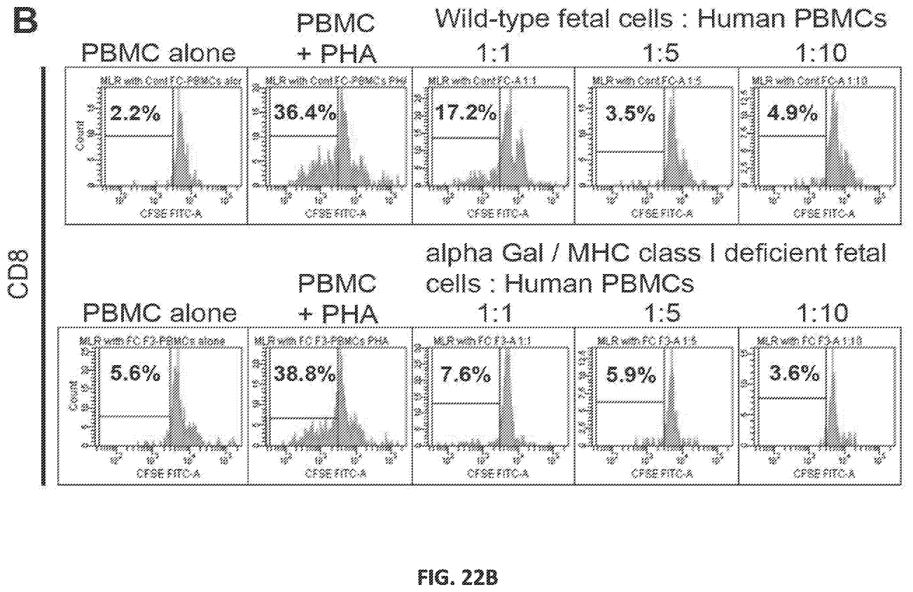

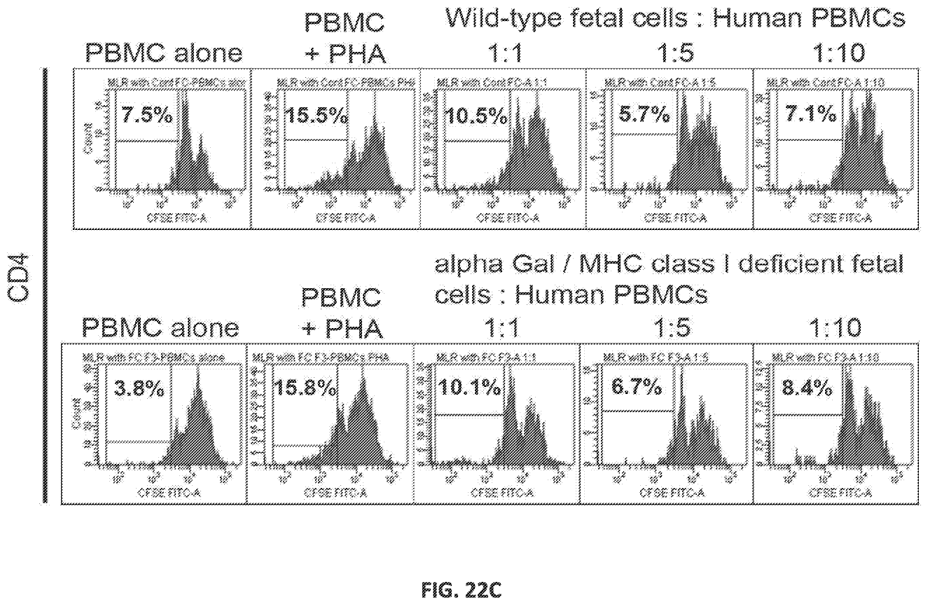

The proliferative response of human CD8+ cells and CD4+ T cells to wild type and GGTA1/NLRC5 knockout fetal cells are shown in FIGS. 22A-22C. Cells were gated as CD4+ or CD8+ before assessment of proliferation (FIG. 22A). CD8 T cell proliferation was reduced following treatments stimulation by fetal cells with GGTA1/NLRC5 knockout fibroblasts compared to wild type fetal cells. Almost 55% reduction in CD8+ T cells proliferation was observed when the human responders were treated with GGTA1/NLRC5 knockout fetal cells at 1:1 ratio (FIG. 22B). Wild type fetal cells elicited 17.2% proliferation in human CD8+ T cells whereas the GGTA1/NLRC5 knockout fetal cells from fetus 3 (pregnancy 1) induced only 7.6% proliferation (FIG. 22B). No differences were observed in CD8+ T cells proliferative response at 1:5 and 1:10 ratio compared to the wild type fetal cells (FIG. 22B). No changes were observed in CD4+ T cell proliferation in response to GGTA1/NLRC5 knockout compared to the wild type fetal cells (FIG. 22C).

Delivery of Live Piglets

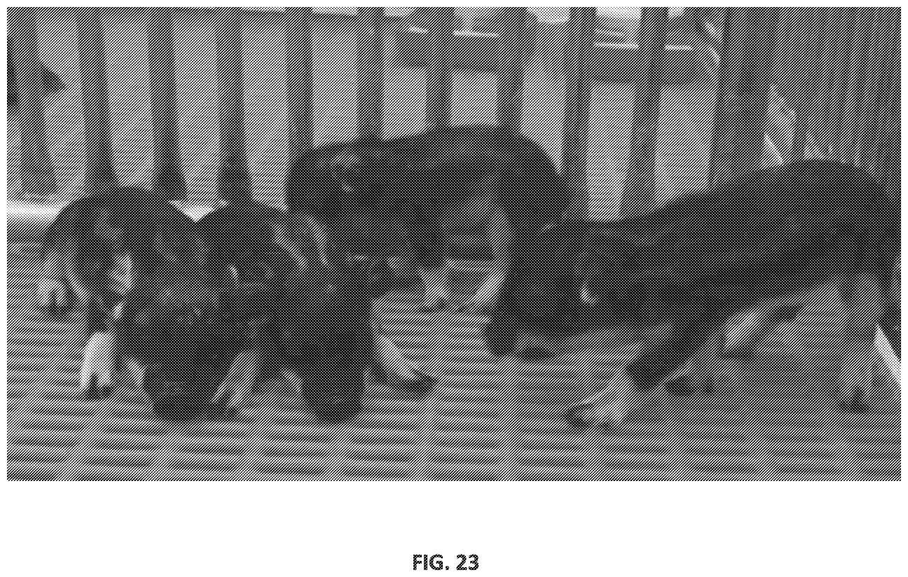

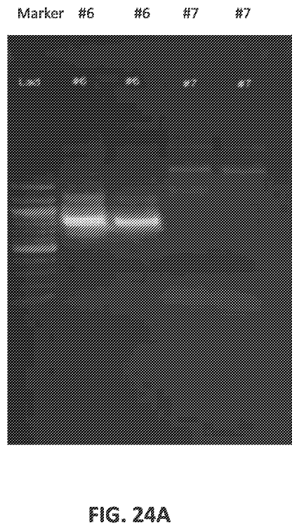

One of the pregnancies obtained above was allowed to complete gestation. 7 live piglets were delivered by C-section at full term (FIG. 23). Ear clippings and tail skin samples were taken and analyzed for screening mutations at or near the GGTA1 and NLRC5 genes. The genotypes of the piglets were determined by PCR. Three PCR experiments using different primer pairs were performed to confirm the genotype of the piglets.

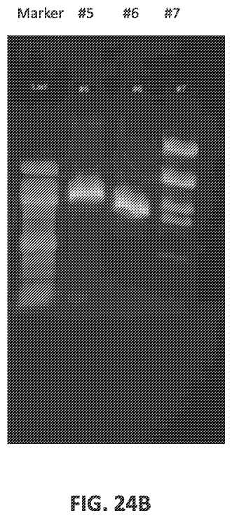

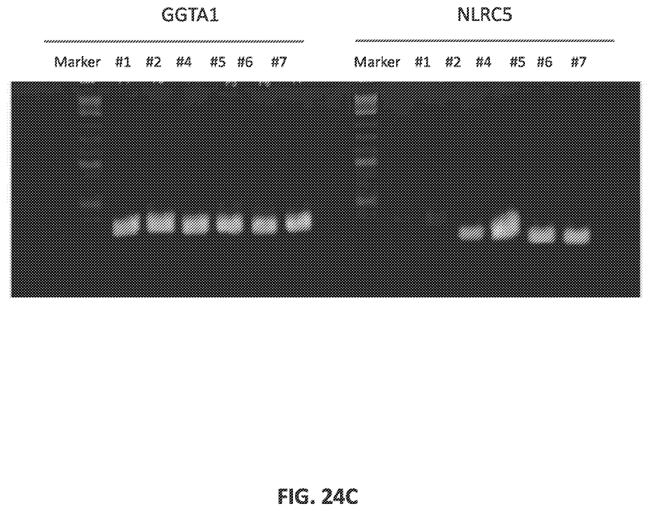

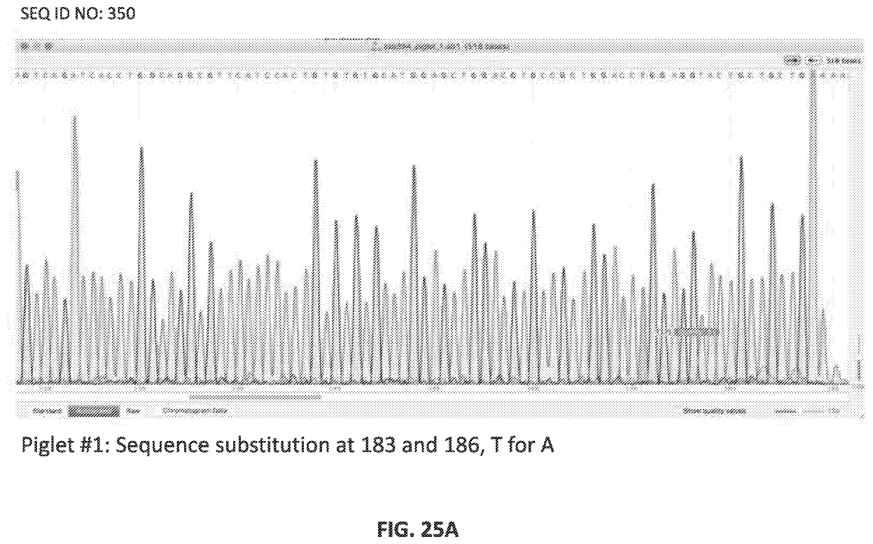

First PCR experiment: PCR was run using samples from piglets #6 and #7. The NLR amplification for piglet #6 produced one strong band, while #7 produced an array of bands when run on a gel (FIG. 24A). The strongest bands were gel extracted from each piglet and yielded sufficient DNA for sequencing. The PCR product of piglet #6 showed robust band at predicted PCR product. The PCR product of piglet #7 showed a band at size different from the predicted PCR product. The results indicated that piglet #6 was a mono-allelic mutant while piglet #7 was a bi-allelic mutant at the NLRC5 gene site. Primer set used for GGTA1 genotyping were: Gal amp 1 forward: gagcagagctcactagaacttg (SEQ ID NO: 153), and Gal amp1 reverse: AAGAGACAAGCCTCAGACTAAAC (SEQ ID NO: 154) (644 bp amplicon). Primer set used for NLRC5 genotyping were: NL1_First_screen Forward: ctgctctgcaaacactcaga (SEQ ID NO: 155), and NLRC5-678 Reverse: gtggtcttgcccatgcc (SEQ ID NO: 156 (630 bp amplicon).

Second PCR experiment: PCR was run using samples from piglets #5, #6, and #7. Only NLRC5 gene was tested. The same PCR amplification as in the first PCR experiment was performed. The PCR product of piglets #5 and #6 showed a band at the expected size (FIG. 24B). The PCR product of piglet #5 showed a second faint band (FIG. 24B). The PCR product of piglet #7 showed an array of bands as in the first PCR experiment above. These results indicated that the NLRC5 gene had in piglets #5, #6, and #7 mono-allelic and bi-allelic mutations in these piglets.

Third PCR experiment: PCR was run using samples from piglets #1, #2, #4, #5, #6, and #7. Primer set used for GGTA1 genotyping were SEQ ID NOs. 193 and 194 in Table 11 (303 bp amplicon). Primer set used for NLRC5 genotyping were SEQ ID NOs. 197 and 198 in Table 11 (217 bp amplicon). The NLRC5 gene amplification for piglets #1 and #2 was not as robust as the rest and produced a fainter band (FIG. 24C). Piglet #5 produced a more smeared band than the rest as well (FIG. 24C). The GGTA1 screen produced consistent bands. The NLRC5 gene amplification product is smaller and different in this experiment and created a product that varied in piglets #1 and #2, #4 and #5, #6 and #7, indicating that different mutations were present that lead to the loss of MHC class 1 expression.

Genotyping the Piglets by Sequencing

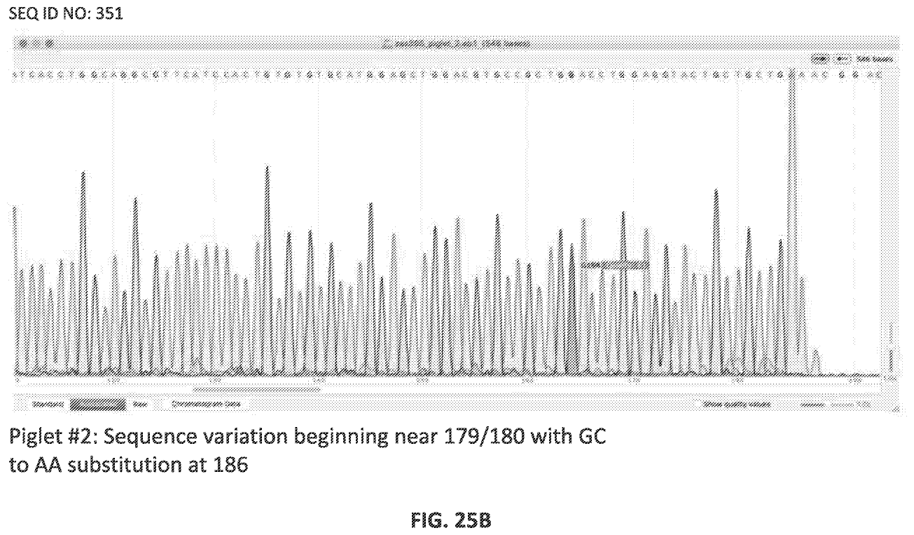

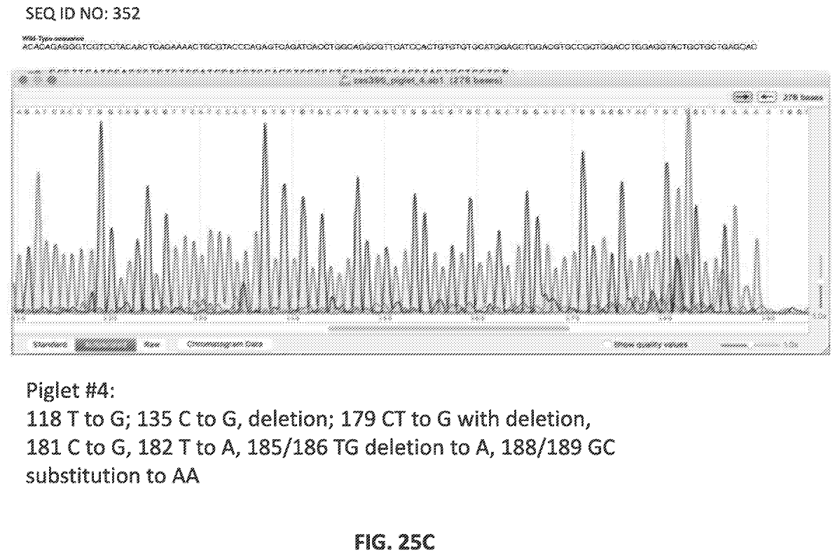

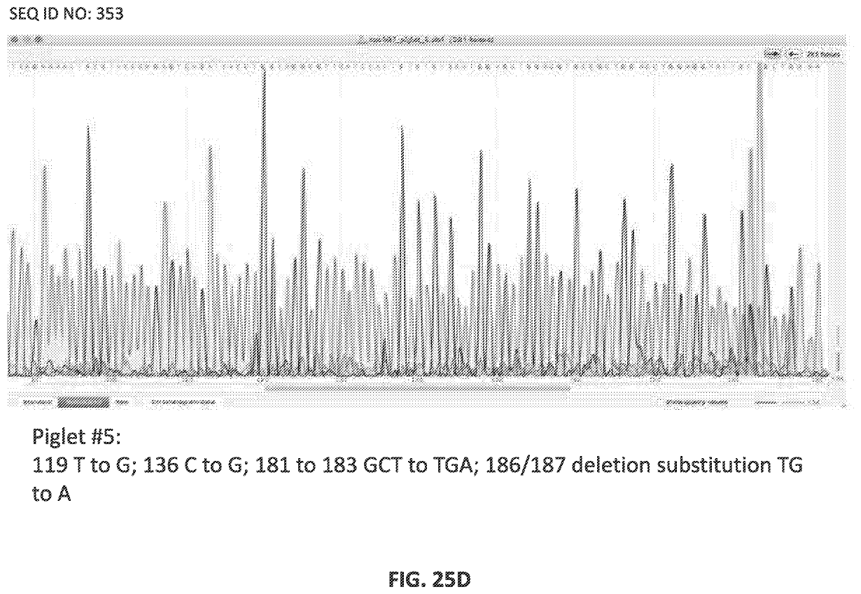

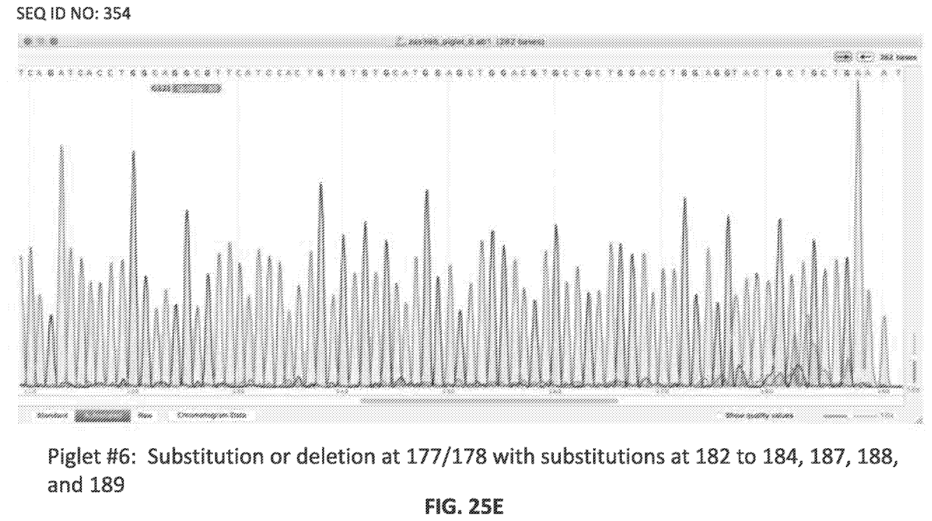

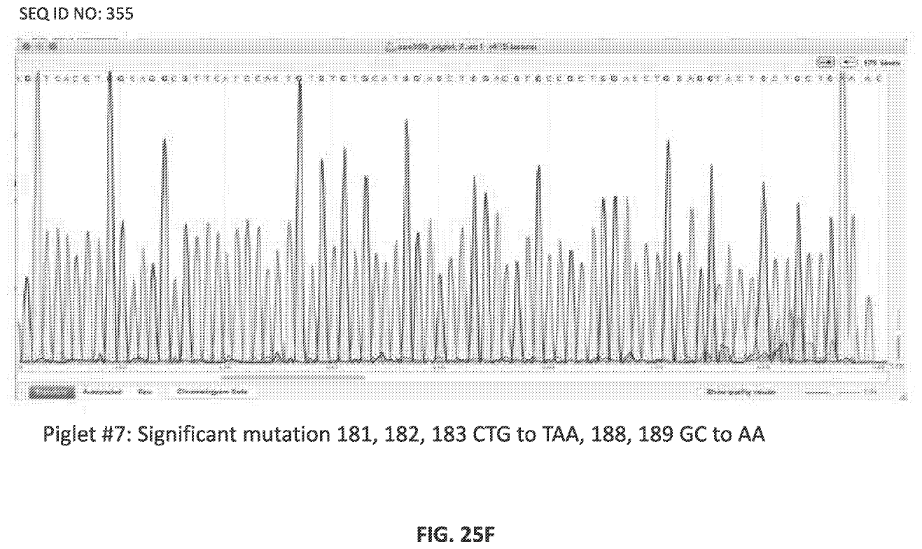

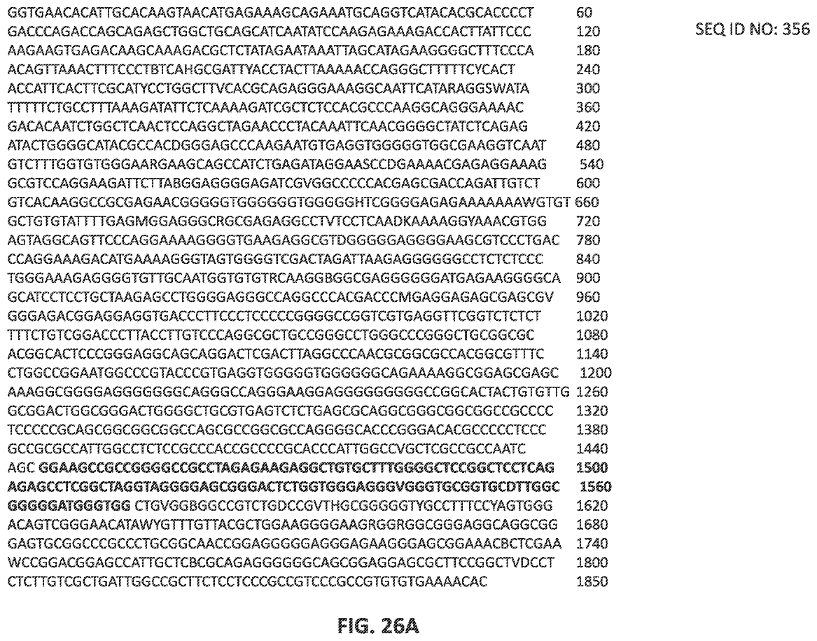

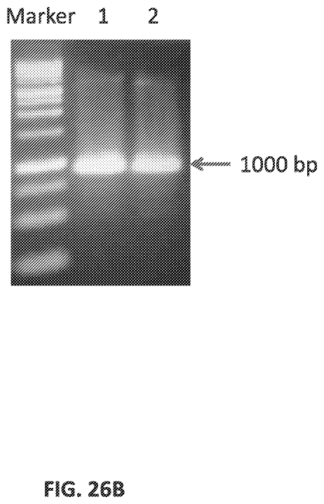

The genotypes of the piglets were determined by sequencing. As shown in FIGS. 25A-25F, Piglets #1, #2, #4, #5, #6, and #7 had one or more mutations on the NLRC5 gene.

### Example 8. Generation and Characterization of GGTA1/NLRC5 Knockout/HLA-G1 Knockin Cells for Making Genetically Modified Pig

One strategy to enhance porcine xenografts survival when transplanted to a recipient (e.g., a primate such as human) is to simultaneously suppress the level of Gal α-(1,3)Gal antigen (Gal antigen) and SLA1, and in the meantime, to suppress the graft-activated natural killer cell (NK cells) proliferation in absence of SLA1. To this end, cells with GGTA1 knocked out (to suppress Gal antigen), NLRC5 knocked out (to suppress SLA1), and HLA-G1 knocked in (to suppress NK cell proliferation) were generated using CRISPR-Cas9-mediated gene editing technology.

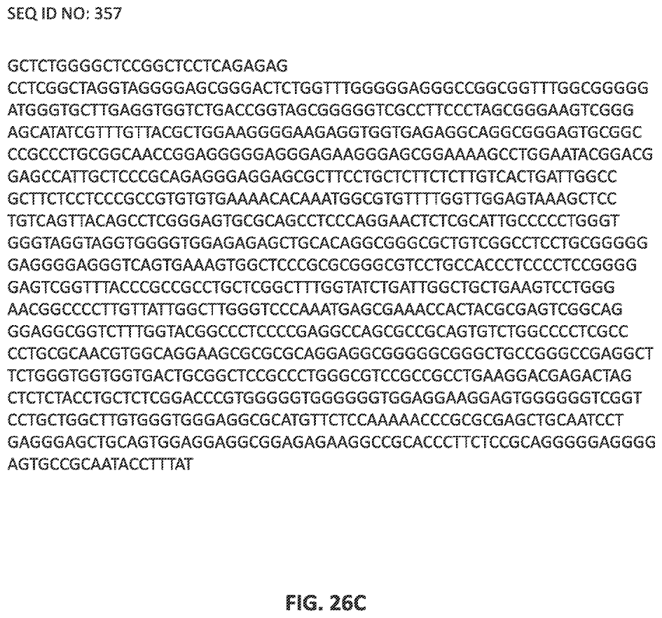

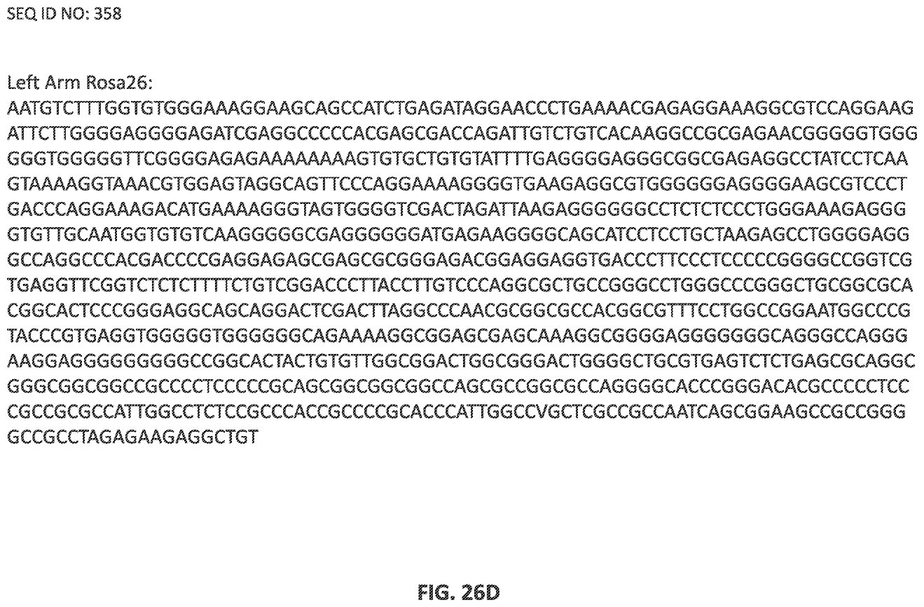

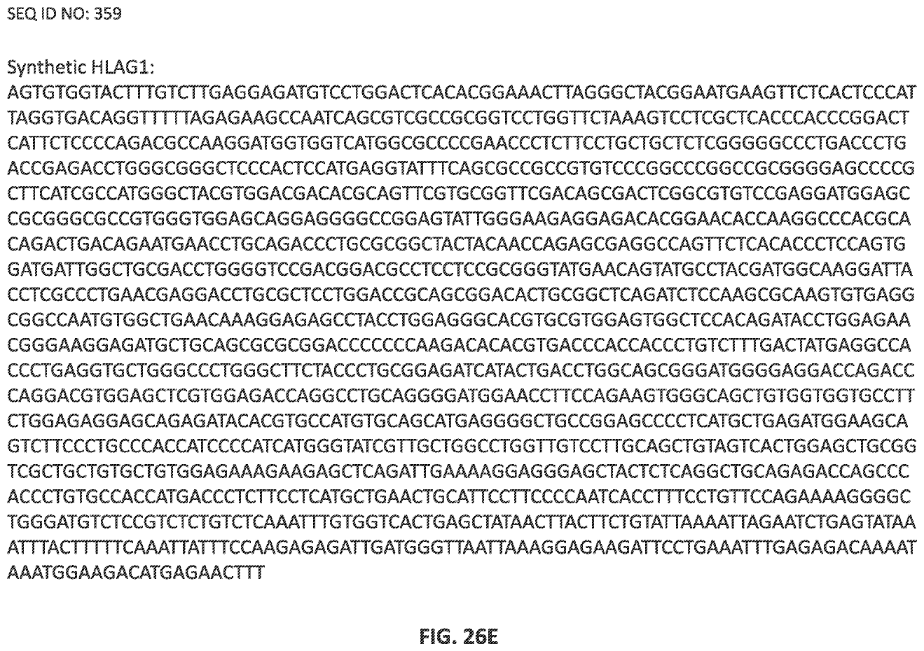

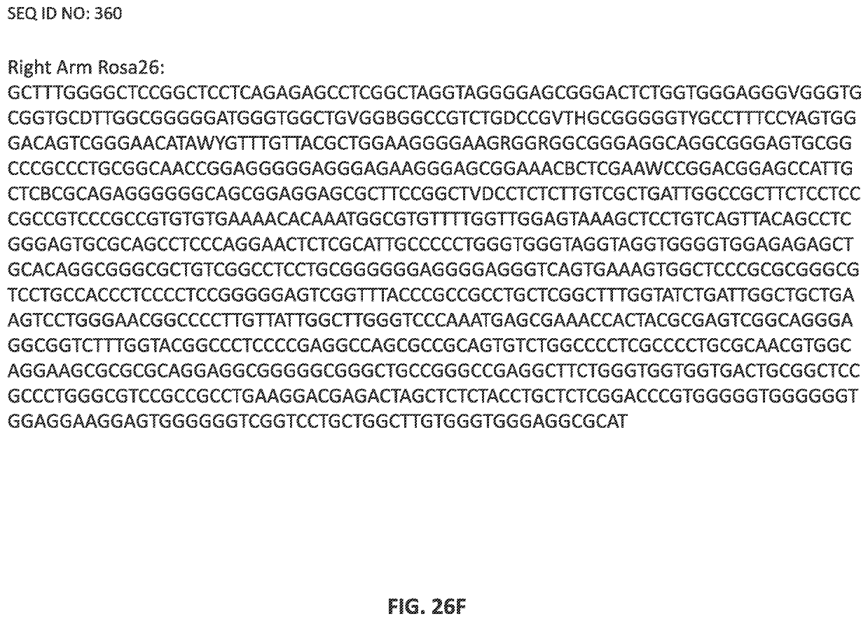

To get the optimal expression of HLA-G1, HLA-G1 cDNA was integrated within the first exon of pig Rosa26. The accurate sequence of exon1 of Rosa26 was determined as described in Example 2 above. We first confirmed the 1000 bp DNA sequence to 5′ and 3′ sequence of the cut site on Rosa26. 1000 bp upstream of the cut site was designed as left homologous arm and 1000 bp downstream was designated as right homologous arm. The sequence of left homologous arm was adapted by Li et al., (Li P. et al., Identification and cloning of the porcine Rosa26 promoter and its role in transgenesis. Transplantation Technology 2014, doi: 10.7243/2053-6623-2-1) (FIG. 26A), which was later on confirmed by amplifying it using sequence specific primers. The primers for right homologous arm (including exon 1 and the cut site) were designed and amplified 1000 bp product based on the sequence available in database using Long Amp (NEB). Following were the primers for the amplification of left and right homologous arms: Left Rosa26 Forward: gcagccatctgagataggaaccctgaaaacgagagg (SEQ ID NO: 157), Left Rosa26 Reverse: acagcctcttctctaggcggcccc (SEQ ID NO: 158); Right Rosa26 Forward: cgcctagagaagaggctgtg (SEQ ID NO: 263) and Right Rosa26 Reverse: actcccataaaggtattg (SEQ ID NO: 264).

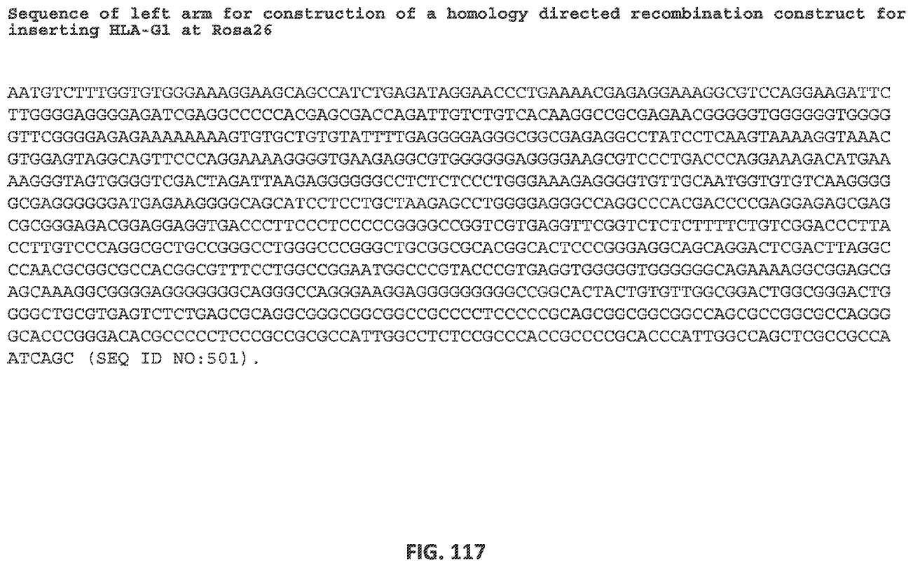

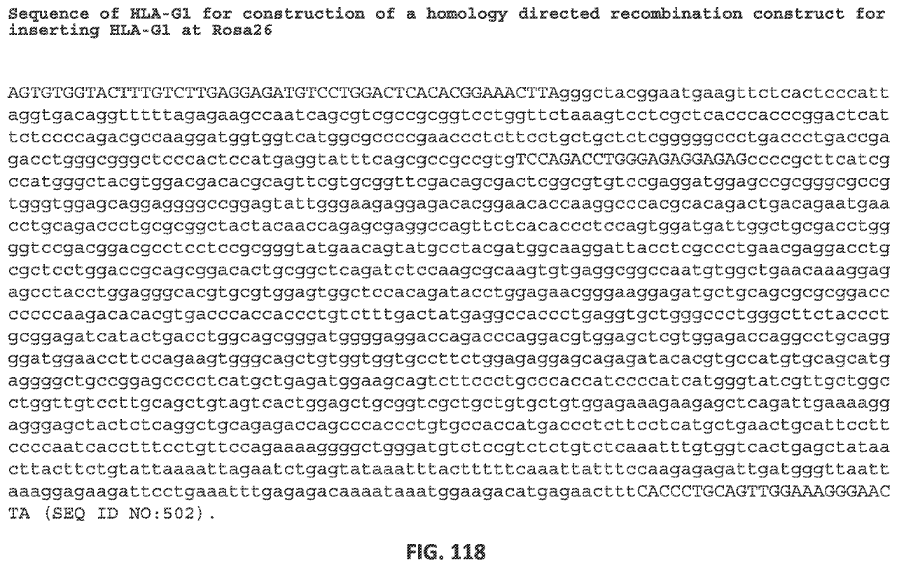

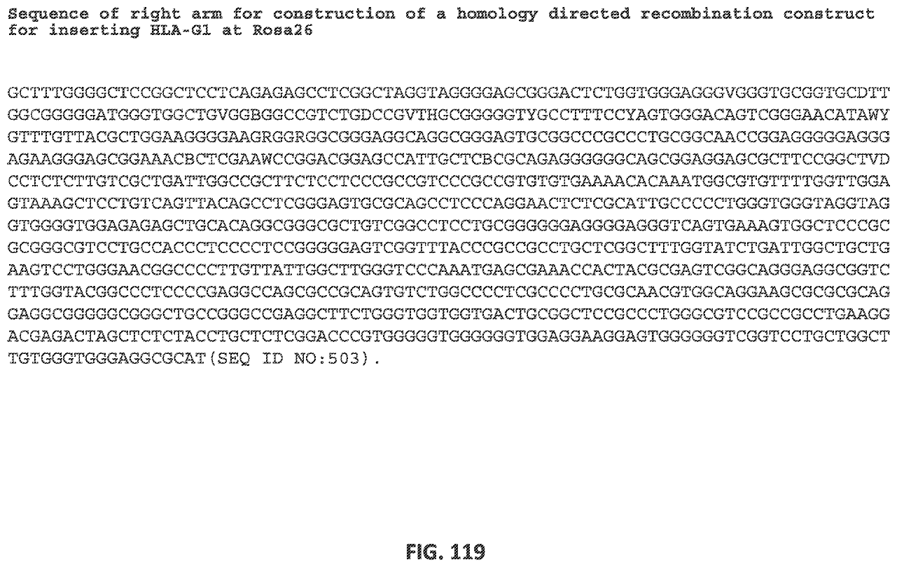

The sequences of the arms were validated by performing next generation sequencing. Amplicon of Rosa26 gene from pig obtained after long range PCR (Qiagen, USA) as per manufacturer's instructions. The amplified product was run of 0.8% of agarose gel (FIG. 26B, Lanes: Marker: 1 kb DNA ladder; 1 and 2 Rosa26 amplicons run in duplicates). The amplified fragments were eluted from gel using Gel extraction kit (Invitrogen, USA) following the manufacturer's instructions. The eluted fragment was quantitated by nano-drop and subjected to Next Gen Sequencing. The consensus sequence of amplicon based on paired read analysis is shown in FIG. 26C. Homology directed recombination constructs for inserting HLA-G1 at Rosa26 locus are shown in FIGS. 26D, 26E, and 26F; and FIGS. 117-119.

Generation of Homology Directing Fragments Containing HLAG1 for Rosa26 Locus

Inserting HLA-G1 at the Rosa26 locus was performed using Gibson Assembly technology, which allowed for successful assembly of multiple DNA fragments, regardless of fragment length or end compatibility, in a single-tube isothermal reaction. The Gibson Assembly Master Mix included three different enzymatic activities that were adopted to perform in a single reaction buffer: the exonuclease created single-stranded 3′ overhangs that facilitated the annealing of fragments that shared complementarity at one end (overlap region); the DNA polymerase filled in gaps within each annealed fragment; and the DNA ligase sealed nicks in the assembled DNA.

PCR was performed for generating homologous left and right arms (having appropriate base overlap with the HLA-G1 sequence). Chemical synthesized gBLOCK for HLA-G1 was re-suspended in nuclease free water at the concentration 10 ng/mL. Since HLA-G1 was large enough to add on 50 bp as an overlapping mark further, we used left and right arms to add an extra 50 bp overlapping to HL-G1. We added the 50 bp overlap in the reverse primer of fragment 1 (Left arm for homology-directed repair (HDR)) and forward primer of fragment 2 (Right arm of HDR). So, left and right arm were 1050 bp in length.

The reaction for the left arm fragment was set up as follows: 2 μL of DNA (concentration 298 ng/ml), 1 μL of Forward Primer (GLF)(10 μM), 1 μL of Reverse Primer (GLR)(10 μM), and 21 μl of Nuclease Free Water (NFW) were mixed. The mixture was added to High Yield PCR EcoDry Premix (obtained from Clontech). PCR was performed. The predicted amplicon size was 1050 bp. The Tm was 61.5° C. The PCR product resulted multiple bands on an agarose gel. The 1050 bp band was eluted from the agarose gel for assembly and for better representation of the image.

The reaction for the right arm fragment was set up as follows: 10 μL of 10× Long Range Buffer, 1 μL of dNTP, 2 μL of DNA (concentration 298 ng/ml), 1 μL of Forward Primer (10 uM), 1 μL of Reverse Primer (10 μM), 2 μL of Long Range Amp were mixed with nuclease free water to make up a total volume of 50 μL. The Tm was 67° C. The expected amplicon size was 987 bp.

The reaction for the middle fragment (HLA-G1) was set up as follows: 10 μL of 10× Buffer, 1 μL of dNTPs, 1 μL or 2 μL of gBlock concentration), 1 μL of Forward Primer (10 uM), 1 μL of Reverse Primer (10 uM), 2 μL of Long Range Amp were mixed with nuclease free water to make a total volume of 50 μL. The Tm was 67° C.

Purification of left, right and middle arms from the agarose gel was done as per instructions of PureLink® Quick Gel Extraction Kit (Invitrogen). The concentrations of all fragments were measured using nano-drop spectrophotomter. 23.5 ng/μL, 30 ng/μL and 28.3 ng/μL were the concentrations of left, middle and right fragments eluted from 1.2% agarose gel. Following the instructions for the Gibson assembly, 2 μL of each fragment was mixed with 10 μL of GA master mix (NEB) and 4 μL of nuclease free water to make the final volume 20 μL in a 0.2 ml of PCR tube and incubated in a thermal cycler at 50° C. for an hour.

Then 2 μL of assembled product was subjected to Long range PCR using Long Amp (NEB) using forward primers of left arms and reverse primers of right arm. The reaction of the Long range PCR was set up as follows: 10 μL of 5× Long Range Buffer, 1 μL of dNTPs (100 μM; NEB), 2 μL of Amplified gblock HLA-G1, 1 μL of Forward Primer (10 μM), 1 μL of reverse Primer (10 μM), were mixed with nuclease free water to a final volume of 50 μL. The PCR was performed and the expected amplicon size was about 3000 bp.

Designing and Cloning of gRNA to Targeting the Exon 1 of Pig Rosa26 Exon 1, GGTA1 and NLRC5 (for SLA1 Knock Out)

Specific oligonucleotides for making gRNAs that make cut in exon 1 of pig ROSA26 exon 1, in close proximity of first codon of GGTA1, or NLRC5 were designed using http://zifit.partners.org/ZiFiT/CSquare9Nuclease.aspx. The cDNA sequence of HLA-G1 is shown in Table 2, and the genomic sequence of HLA-G is shown as SEQ ID: No. 191. The maps of the genomic sequence and cDNA of HLA-G are shown in FIGS. 14A-14B.

Briefly, the oligonucleotides were synthesized and resuspended in respective amount of nuclease free water to get the concentration of 100 μM each. 1 μL of each oligonucleotides (forward and reverse) were mixed with 1 μL of 10×T4 Polynucleotide Kinase Reaction Buffer, 0.5 μL T4 Polynucleotide Kinase and 6.5 μL of dH₂O to make up the total volume 10 μL in 0.2 μL tubes. The tubes with the reaction solution were placed in a thermocycler. The following program was run for the appropriate annealing of the forward and reverse oligos: 37° C. for 30 min; 95° C. for 5 min; Ramp down to 25° C. at 0.1° C./sec. The annealed oligos were diluted by 1:100.

Plasmid pX330-U6-Chimeric BB-CBh-hSpCas9 (Addgene) was used to clone the annealed oligonucleotides to generate gRNA for the CRISPR-associated Cas9 nuclease system. One microgram of plasmid pX330 was digested with Bbsl (New England Biolabs, Ipswich, Mass.) for 15 min at 37° C. using fast digest buffer and then kept for 15 min to inactivate Bbsl. Then 0.2 μL of Calf-intestinal alkaline phosphatase (CIP) was added and incubated for 1 hour, to avoid the self-ligation of the digested vector. Digested pX330 was purified using Plasmid Extraction mini-prep kit (Qiagen). Digested vector was mixed with 300 μL of PB buffer and then added to purification column of this kit, which was then spun down at 8000 rpm×1 min. The flow-through was discarded and the column was washed by PE buffer (containing absolute ethanol) and finally eluted in 50 μL of EB buffer. 1.34 (50 ng) of digested px330 vector was mixed with 1.0 μL diluted oligonucleotides, 5 μL 10×T4 Ligase Buffer, and 2.5 μL T4 DNA Ligase, and the finally the volume was made to 50 μL by adding 39.9 μL of nuclease free water. Negative control was run without adding any oligo to the reaction mix. Ligase was then inactivated at 65° C. for 5 min before heading to transformation in TOP10 competent cells (Invitrogen), following the manufacturer's protocol. The DNA clones were sequence.

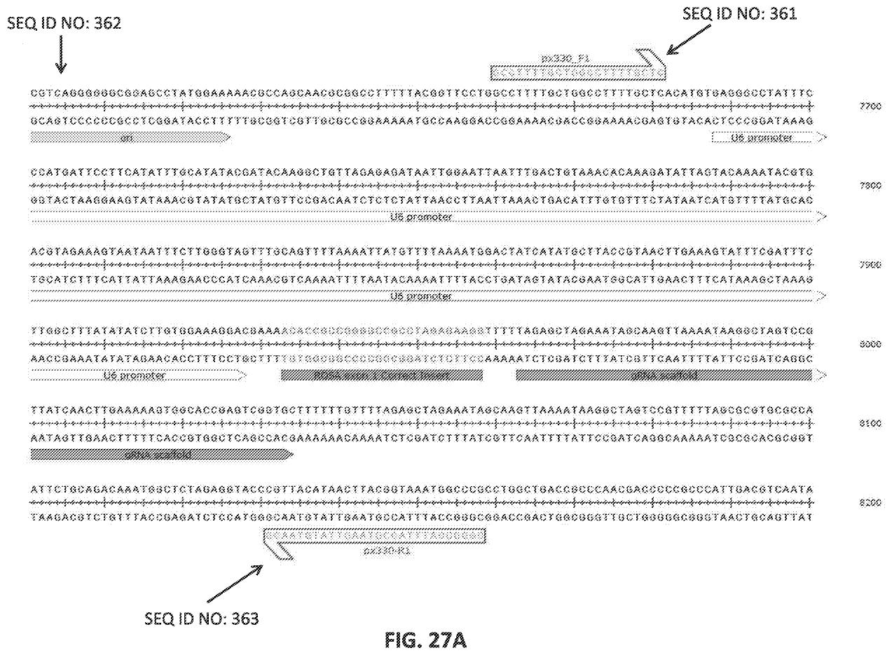

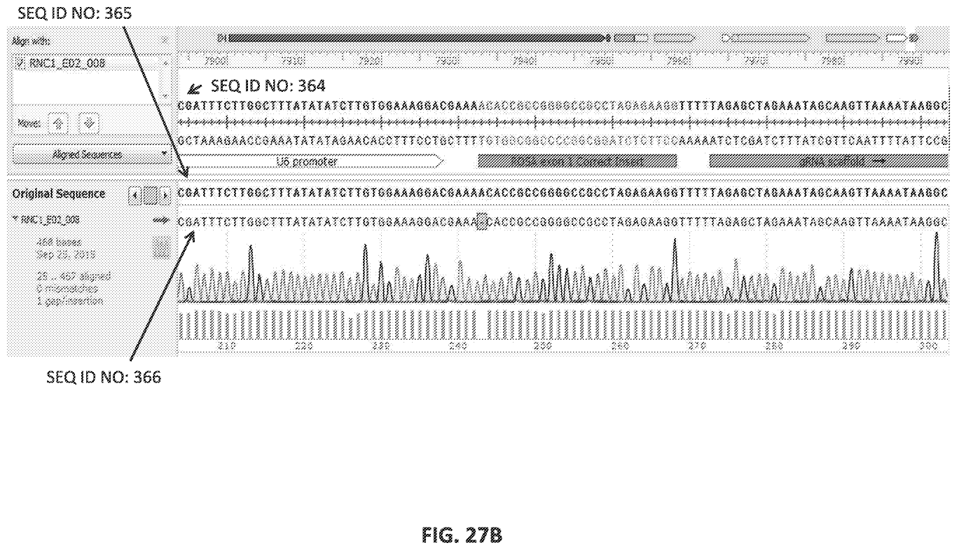

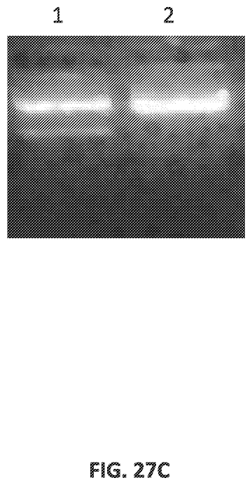

Evidence of ligation of Rosa26 oligos in px330 vector juxtaposed with gRNA was shown in FIGS. 27A, 27B, and 27C. The sequence of the correct clone was shown in FIG. 27A and the RNC1 E02_008 sequencing result of constructed plasmid was shown in FIG. 27B.

Restriction digestion of ligated products was also performed to verify the success of ligation. Two restriction enzymes (Bsbl and Agel) were used to digest the purified the ligated products. Since oligonucleotides were ligated in Bsbl sites, the Bsbl site was disrupted in px300 vectors that harboring oligonucleotides (FIG. 27C, Lane 1: intact vector; Lane 2: ligated vector with disrupted Bsbl site).

In vitro transcription (IVT) and in vitro Cas9-mediated cleavage of target DNA

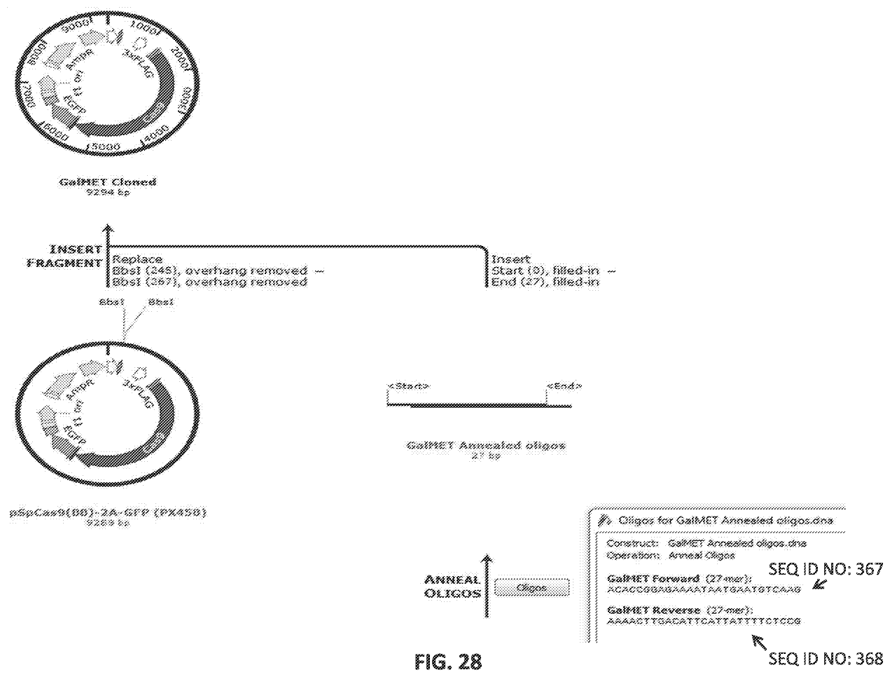

To examine the cleavage potentials of gRNAs designed for Rosa26, GGTA1 and NLRC5 sites, Guide-it™ sgRNA In Vitro Transcription and Screening System was used for the in vitro transcription of guide RNAs following manufacturer's protocol. The respective cleavage potential of the guided RNAs was also examined. The gRNA for GGTA1 cleavage was performed using the GalMet oligos (Forward: acaccggagaaaataatgaatgtcaag (SEQ ID NO: 367); Reverse: aaaacttgacattcattallllctccg (SEQ ID NO: 368)) (FIG. 28). Gal(Met) targeted the first methionine of the GGTA1 cDNA transcript, but not any other regulatory methyl group outside in the promoter region.

A: Amplification of Target (about 2000 kb) for gRNAs.

About 2 kb long amplicon containing target sequence for gRNA for Rosa26, GGTA1 and NLRC5 were amplified using specific primers as per instructions of the kit. Pig DNA and primers were mixed with nuclease free water to a total volume of 25 μL. The mixture was later mixed with Dry PCR mix. The Tm of reactions for Rosa26, GGTA1 and NLRC5 were 61.5° C., 60° C., and 63° C., respectively. All the amplicons from agarose gel was eluted using of Purlink; Quick Gel Extraction kit (Invitrogen).

B: In-Vitro Transcription

Chemically synthesized DNA template that contained designed sgRNAs encoding sequence under the control of a T7 promoter and universal gRNA sequence were obtained from IDT. The template was amplified by PCR with the d Guide-it Scaffold Template provided in the kit.

The IVT templates for Rosa26, NLRC5 and GGTA1 were as follows:

Rosa26:
(SEQ ID NO: 233)
gccgcctctaatacgactcactatagggccgccggggccgcctagagagt
tttagagctagaaatagca;
NLRC5:
(SEQ ID NO: 234)
gccgcctctaatacgactcactatagggccggcctcagaccccacacaga
ggttttagagctagaaatagca;
GGTA1:
(SEQ ID NO: 235)
gcggcctctaatacgactcactataggggagaaaataatgaatgtcaagt
tttagagctagaaatagca.

The 5 μl of Guide-it Scaffold Template (provided in kit) with 1 μl of the abovementioned templates were mixed at a concentration of 10 μM, and dilute with RNase-free water to a final volume of 25 μl. The solutions were mixed by gentle pipetting. The entire 25 μl mixture was added to one tube of High Yield PCR EcoDry Premix. Thermal cycling using the following program: 95° C. for 1 min; 33 cycles of 95° C. for 30 sec, 68° C. for 1 min, 68° C. for 1 min.

The resulting PCR products were run on a 1.8% agarose gel. A single band at about 140 bp was obtained for each of the three IVT templates. The bands were then purified by NucleoSpin Gel provided by the kit.

In vitro transcription was then performed by mixing 100 ng of the PCR products with Guide-it In Vitro Transcription Buffer and Guide-it T7 Polymerase Mix. The final volume was 20 μL by adding nuclease free water and incubated at 42° C. for 1 hour.

C: Purification and Quantification of In Vitro transcribed sgRNA

(1) 2 μl of RNase free DNase I was added to the entire 20 μl of the in vitro transcription reaction and incubate at 37° C. for 0.5 hour.

(2) RNase free water was added to the reaction mixture to a final volume of 100 μl.

(3) 100 μl of phenol:chloroform:isoamyl alcohol (25:24:1), saturated with 10 mM Tris, pH 8.0, 1 mM EDTA was added to the diluted reaction mixture from Step (2) and vortex well.

(4) The solution was centrifuged at 12,000 rpm for 2 min at room temperature. The supernatant was transferred to a new tube, to which an equal volume of chloroform was added.

(5) The solution was vortexed well and then centrifuged at 12,000 rpm for 2 min at room temperature.

(6) The supernatant was transferred to a new tube, added 1/10 volume of 3M sodium acetate and an equal volume of isopropanol, and vortexed well.

(7) The solution from Step (6) was incubated for 5 min at room temperature, and then centrifuged at 15,000 rpm for 5 min at room temperature.

(8) The supernatant was removed carefully. The pellet was rinsed with 80% ethanol and centrifuged at 15,000 rpm for 5 min at room temperature.

(9) The pellet was air dried for about 15 min and resuspended in 20 μl of RNase free water and the concentration was checked using nano-drop.

D: Cas9 mediated cleavage of 2 kb template (section A) with purified gRNAs of Rosa26, NLRC5 and GGTA1

(1) Cleavage reactions containing above sgRNA (specific for a target) and the amplified experimental templates (about 2 kb long of each gene; Rosa26, NLRC5 (NL1) and GGTA1, containing target sequence) were set up.

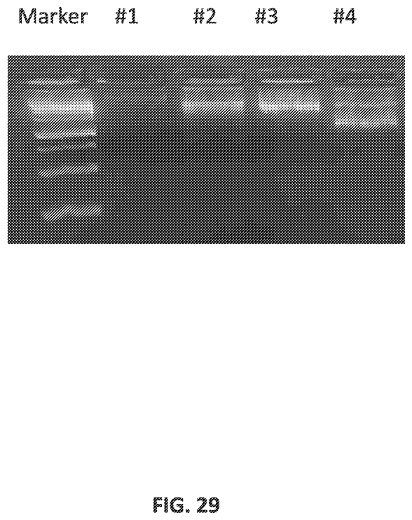

(2) The experimental cleavage template (100 ng total) with experimental sgRNA sample (20 ng total from above), 1 μL of Guide-it Recombinant Cas9 Nuclease, 1 μL of 10× Cas9 Reaction Buffer, 1 μL of 10×BSA were mixed and made up to a final volume of 10 μL with nuclease free water. The mixture was incubated at 37° C. for 1 hour. The reaction was stopped by incubating solution at 70° C. for 10 min. The entire 10 μl of reactions was analyzed on a 1% agarose gel alongside a negative control (100 ng of an uncleaved 2 kb control fragment) (FIG. 29).

Electroporation and Flow Sorting

Cryopreserved cells were seeded 1×10⁶ cells per petri dish in 10% complete DEMEM media. After that the cells were seeded, the media was changed after each 24 h and allowed the petri dish to be confluent (>70%). Then the cells were harvested using PBS, TRYPLE Express and then resuspended in 100 μL of R buffer provided by Neon system for electroporation. 1.5 μg of px330 plasmids containing gRNAs (for Rosa26, GGTA1 or NLRC5) was added in the 1.5 ml tube and mixed by gentle tapping. Afterwards electroporation was performed in a 100 μL tube at 1300 V×30 ms×1 pulse. Cells were seeded in 15% complete DMEM media and monitored after each 12 h. After 12 h post electroporation, the signs of cells adherence were visible.

Pig fetus fibroblasts were electroporated with px330U6-gRNA (met, GGTA1); px330U6-gRNA (Rosa26) and px330U6-gRNA (NLRC5); and amplicon of Gibson assembled HLA-G1 with Rosa26 homologous left and arms (designed for the insertion at the pig Rosa26 locus) were harvested at 5th day after the transfection using 1×PBS −/− and Triple Express.

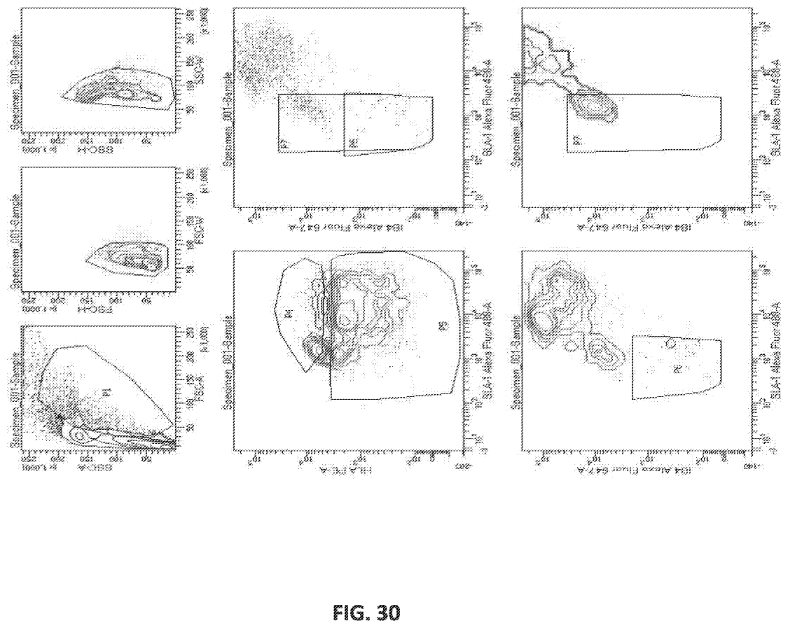

We transfected in three different tubes of and recovered about 1×10⁶ cells from each petri dish. These cells were stained with 1 μg of IB4-APC (Biolegend), 1 μg of anti-pig SLA1-FITC (Novus Biologicals), 5 μL of anti-HLA-G1-PE in 100 μL of flow buffer (PBS-1% BSA), and incubated at 4° C. for 30 min in dark. Negative unstained control was also kept at 4° C. and was subjected to all treatments as we did in stained cells. Also, we made single stained tubes: IB4-APC and SLA1-FITC for the positive control of the respective fluorochromes. After that the pig fibroblasts were spun at 2000 rpm for 5 min in microfuge to remove extra antibodies. Next the cells were resuspended again in flow buffer (100 μL) and passed through flow tubes with strainer (BD). After staining, we capped all the tubes carefully to avoid the chances of contamination while traveling to Flow sorting facility (CCRB, University of Minnesota). The collection media was 2.5% complete DMEM (Pen-Step, Glutamax and FBS) per the instructions of the flow sort core facility. The sorting results are shown in FIG. 30.

Delivery of Live Piglets

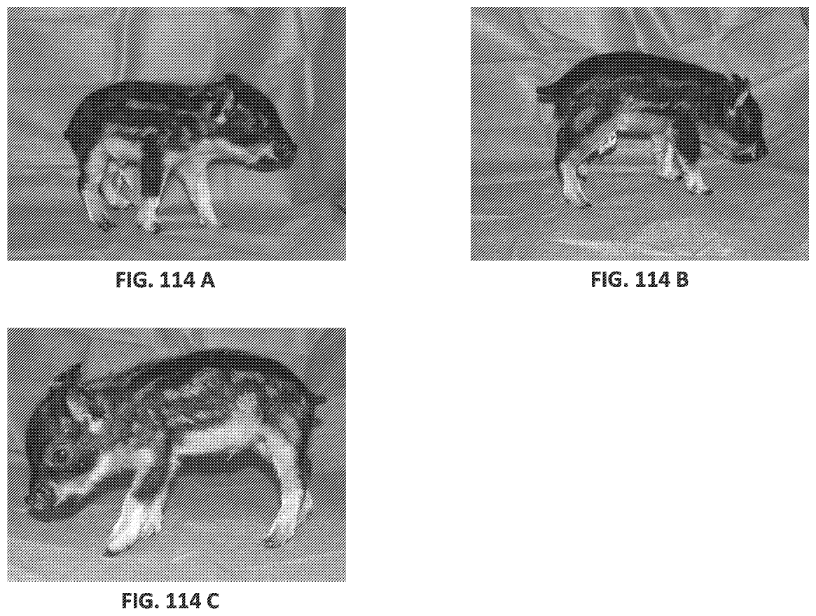

FIG. 114 A-C shows pictures of live births of GGTA1/NLRC5 knockout/HLA-G1 knockin piglets.

Genotyping by Sequencing

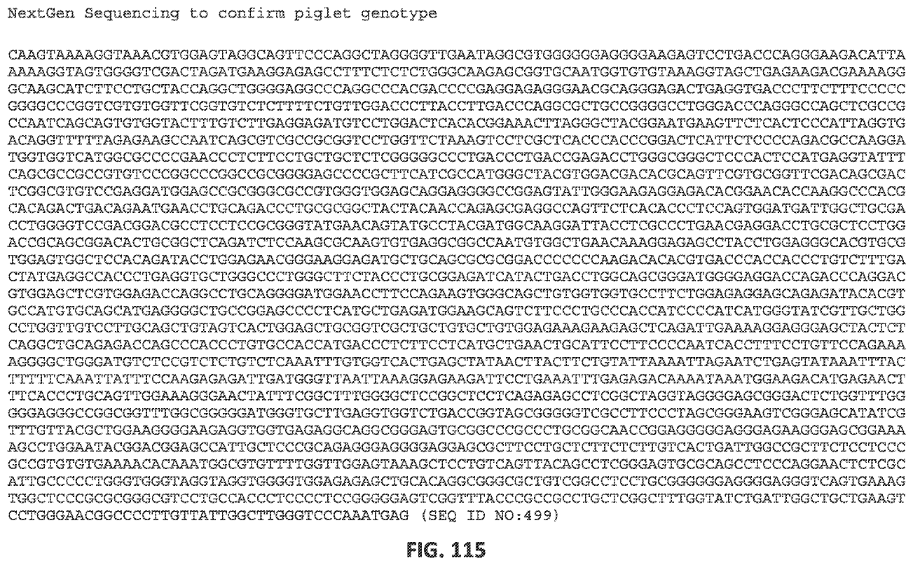

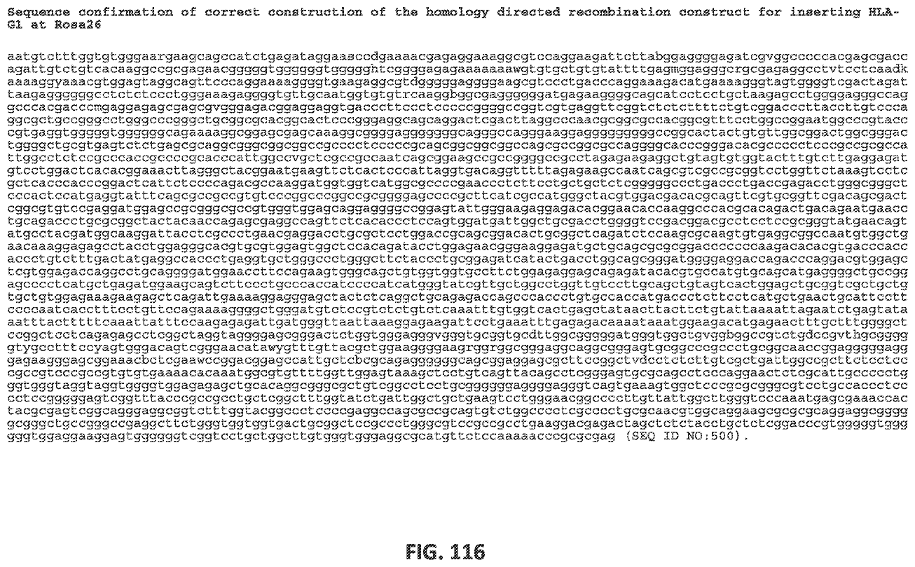

Next generation sequencing was performed to confirm the correct insertion of the HLA-G1 sequence into the ROSA site. The sequencing was performed on a skin sample taken from a live piglet. The confirmed sequence of the HLA-G1 knockin in the ROSA site is shown in FIG. 115 (SEQ ID NO: 499).

### Example 9. Generation and Characterization of GGTA1 Knockout/CD47 Knockin Cells for Making Genetically Modified Pig

One strategy to enhance porcine xenografts survival when transplanted to a recipient (e.g., a primate such as human) is to simultaneously suppress the level of Gal alpha-(1,3)Gal antigen (Gal antigen) and suppress activation of macrophages. To this end, cells with GGTA1 knocked out (to suppress Gal antigen) and human CD47 knocked in (to suppress macrophage activation) were generated using CRISPR-Cas9-mediated gene editing technology.

The GGTA1 Knockout/CD47 knockin cells were generated using similar methods as described in Example 26. GGTA1-targeting gRNA vector, in which GGTA1-specific gRNA (having binding site in exon 1) was cloned under U6 promoter in px330, was transfected with a Gibson assembled GGTA1-CD47 gene hybrid. In the GGTA1-CD47 gene hybrid, CD47 gene was sandwiched between 1000 bp homologous arms (the 5′ side and the 3′ side of cut site) of GGTA1.

CD47 cDNA was assembled with a left arm and a right arm of GGTA1 locus.

The primers for the assembly were: CD47 assembly right forward primer: ttgagcctgtgcatcgcagcgt (SEQ ID NO: 236; CD47 assembly right reverse primer: ctacttttaatgcaagctggtgacttggctgataactagg (SEQ ID NO: 237); CD47 assembly left forward primer: aaattaaggtagaacgcactccttagcgctcgt (SEQ ID NO: 238); CD47 assembly left reverse primer: attttgggcttccatgttggtgacaaaacaaggg (SEQ ID NO: 239).

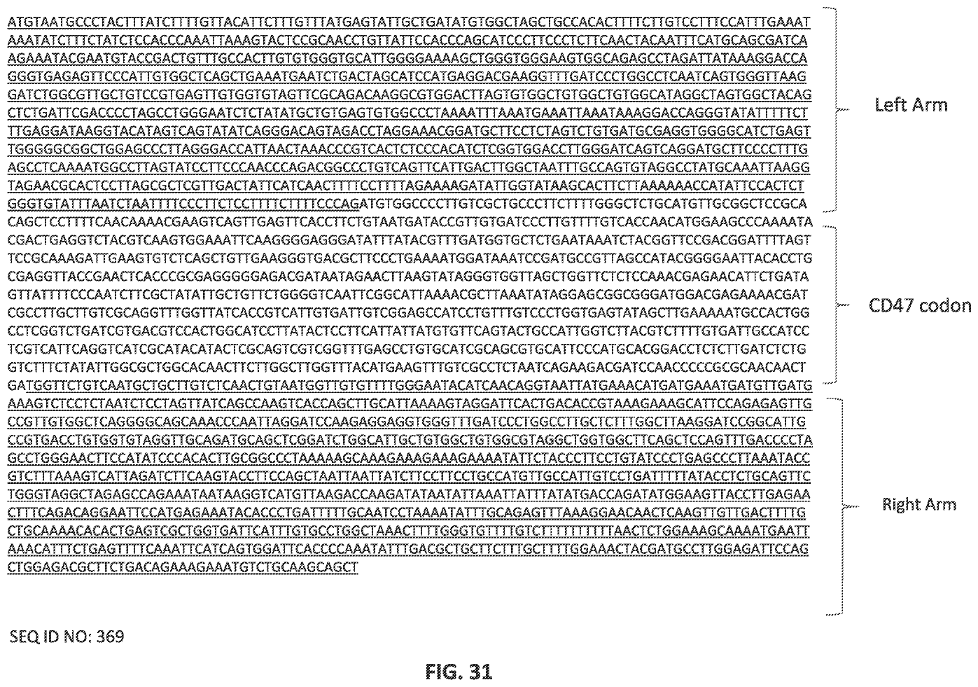

The sequence of resulting assembly construct comprising the left arm, CD47 coding sequence, and the right arm was shown in FIG. 31 (left arm and right arm underlined). The CD47 sequence was optimized for pig codon usage and was made synthetically and assembled. This sequence was not derived from human cells. It was designed to express with the correct amino acid profile in pigs. The CD47 sequence (Table 12) was optimized for pig codon usage and was made synthetically and assembled.

TABLE 12
Synthetic CD47 for expressing in pig

| | |
|---|---|
| SEQ | atgtggccccttgtcgctgcccttcttttgggctctgcatga |
| ID | gcggctccgcacagctccttttcaacaaaacgaagtcagttg |
| NO | agttcaccttctgtaatgataccgagtgatcccttgattgtc |
| 240 | accaacatggaagcccaaaatacgactgaggtctacgtcaag |
| | tggaaattcaaggggagggatatttatacgtttgatggtgct |
| | ctgaataaatctacggttccgacggattttagttccgcaaag |
| | attgaagtgtctcagctgagaagggtgacgcttccctgaaaa |
| | tggataaatccgatgccgttagccatacggggaattacacct |
| | gcgaggttaccgaactcacccgcgagggggagacgataatag |
| | aacttaagtatagggtggttagctggttctctccaaacgaga |
| | acattctgatagttattttcccaatcttcgctatattgctgt |
| | tctggggtcaattcggcattaaaacgcttaaatataggagcg |
| | gcgggatggacgagaaaacgatcgccttgcttgtcgcaggtt |
| | tggttatcaccgtcattgtgattgtcggagccatcctgtttg |
| | tccctggtgagtatagcttgaaaaatgccactggcctcggtc |
| | tgatcgtgacgtccactggcatccttatactccttcattatt |
| | atgtgttcagtactgccattggtcttacgtcttttgtgattg |
| | ccatcctcgtcattcaggtcatcgcatacatactcgcagtcg |
| | tcggtttgagcctgtgcatcgcagcgtgcattcccatgcacg |
| | gacctctcttgatctctggtctttctatattggcgctggcac |
| | aacttcttggcttggtttacatgaagtagtcgcctctaatca |
| | gaagacgatccaacccccgcgcaacaactga |

The CD47 gene was targeted to the GGTA1 gene cut site with left and right arms that are homologous to the GGTA1 gene. The GGTA1 gene was inactive in adult islets but the promoter was turned on in blood cells and splenocytes of adult pigs. Therefore, a CD47 expressing pig (from the GGTA1 site) will be a great vaccine donor.

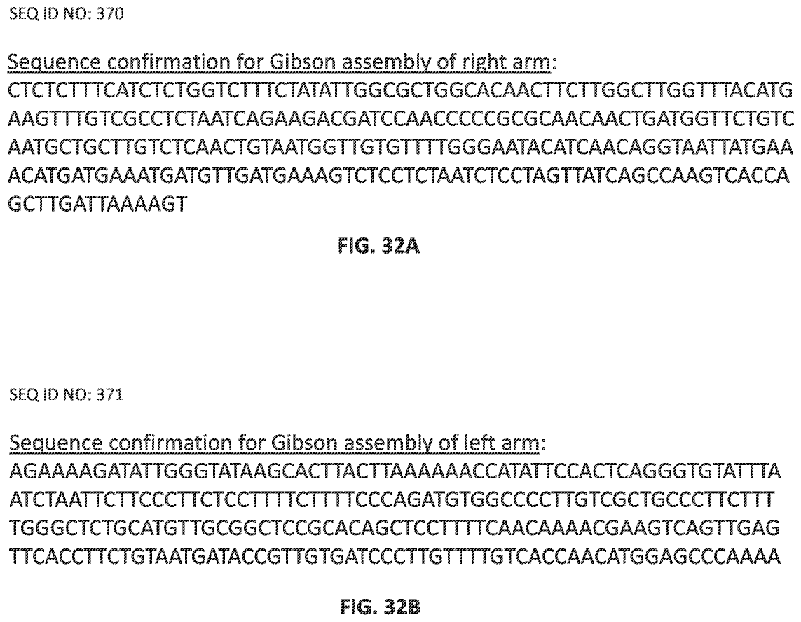

The assembly was confirmed by sequencing. The sequences of the assembled left arm and right arm are shown in FIG. 32.

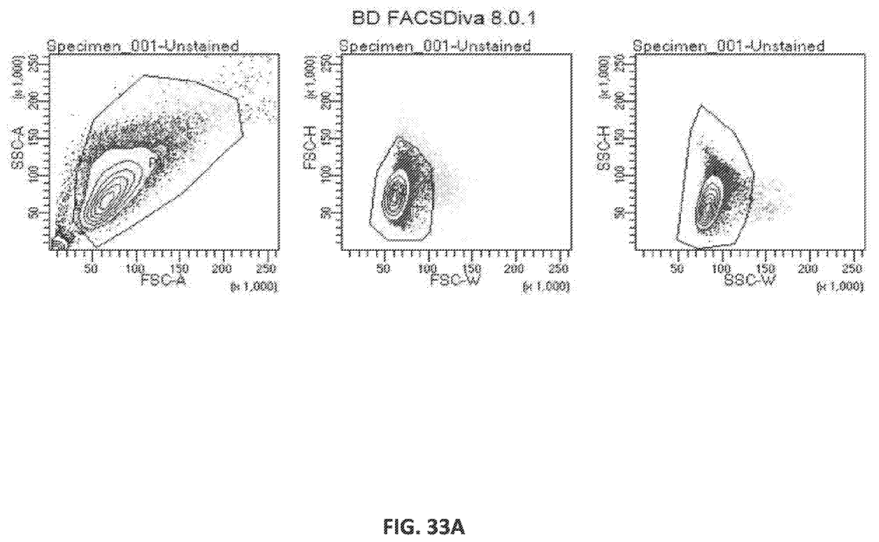

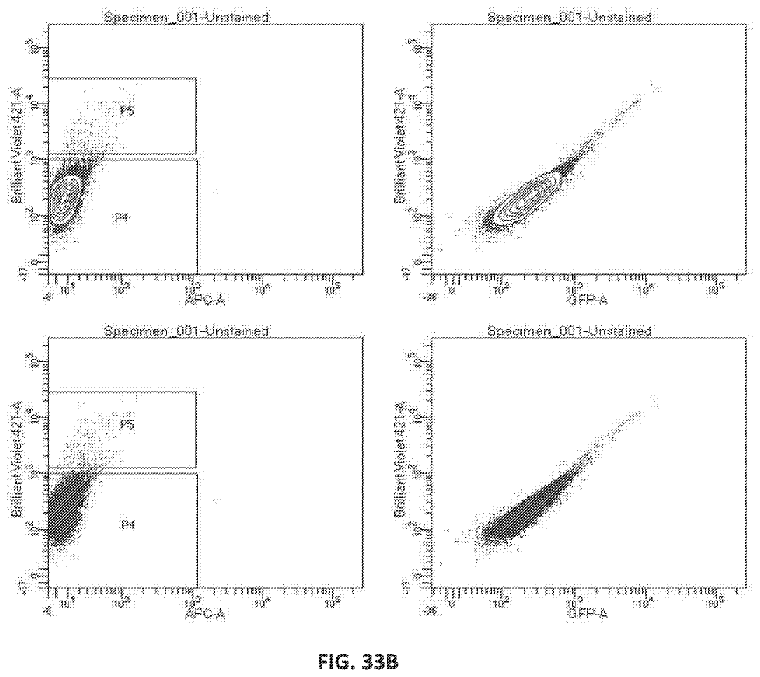

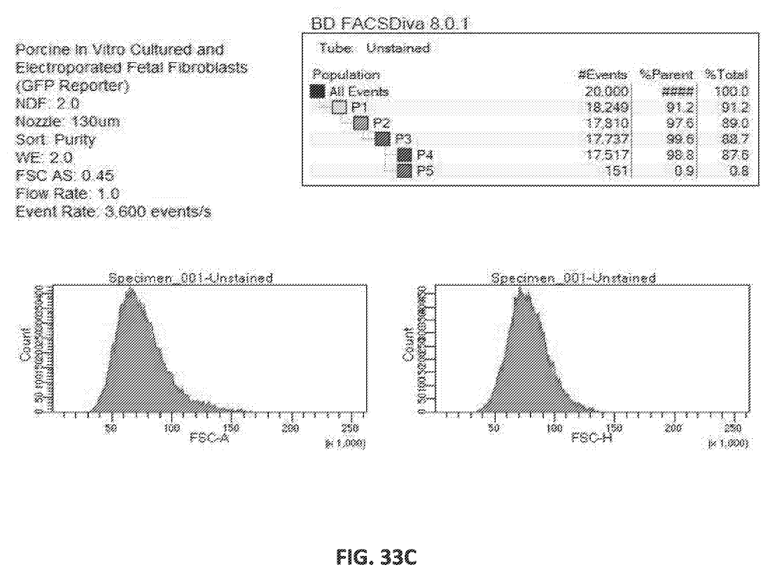

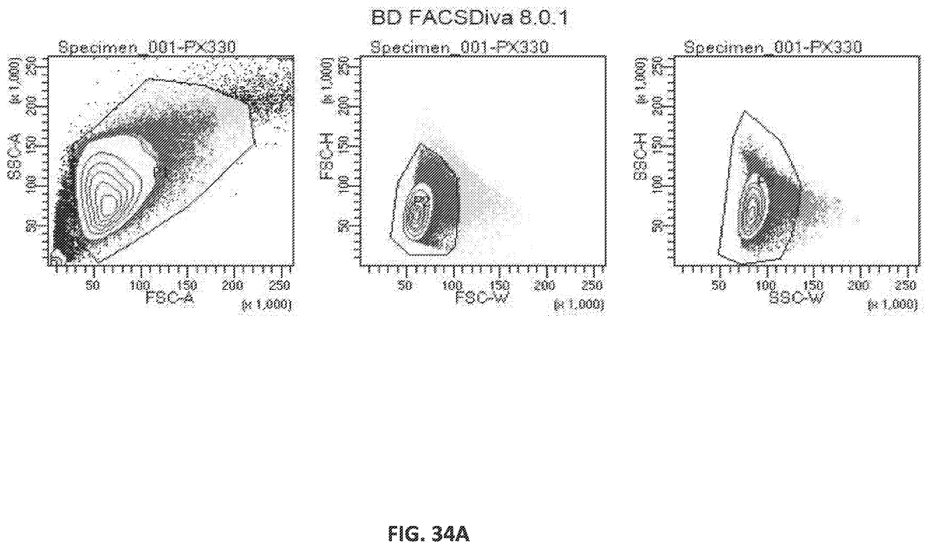

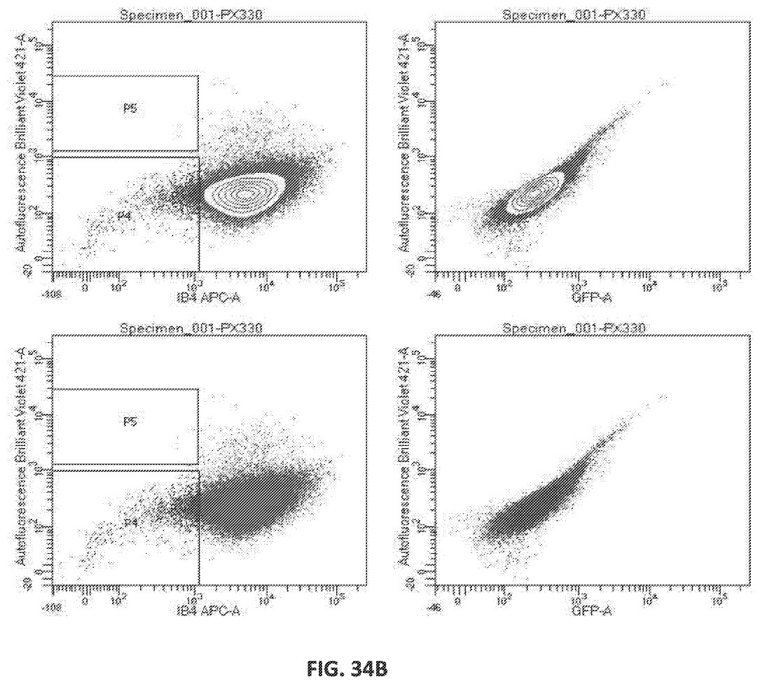

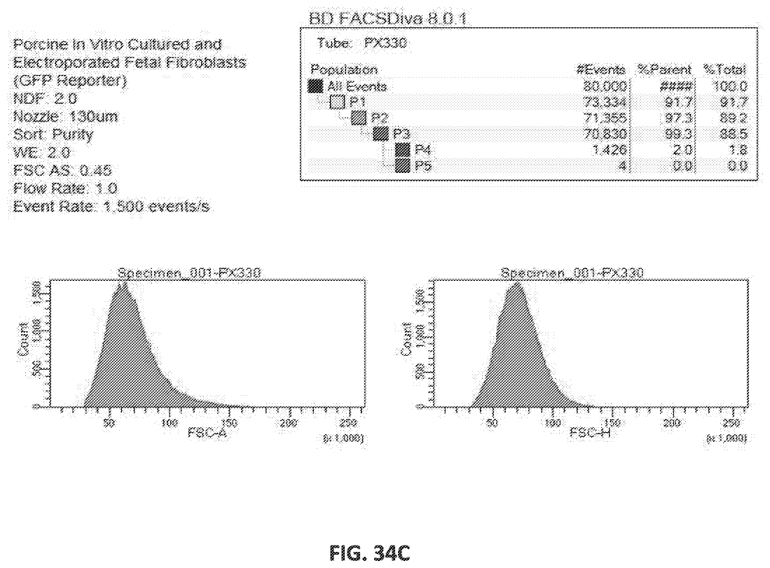

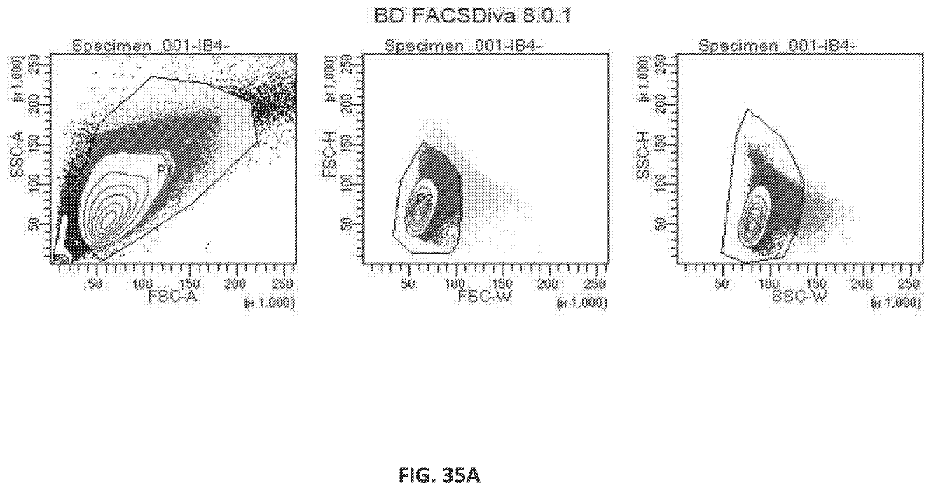

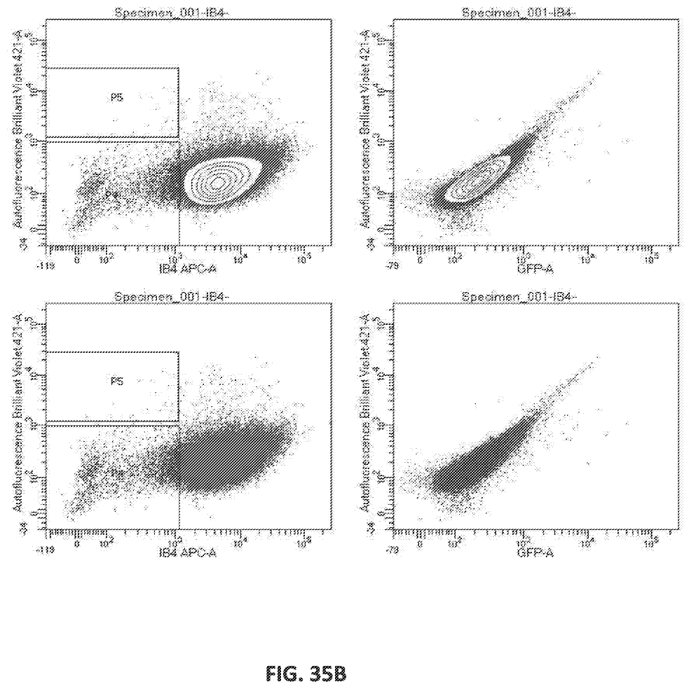

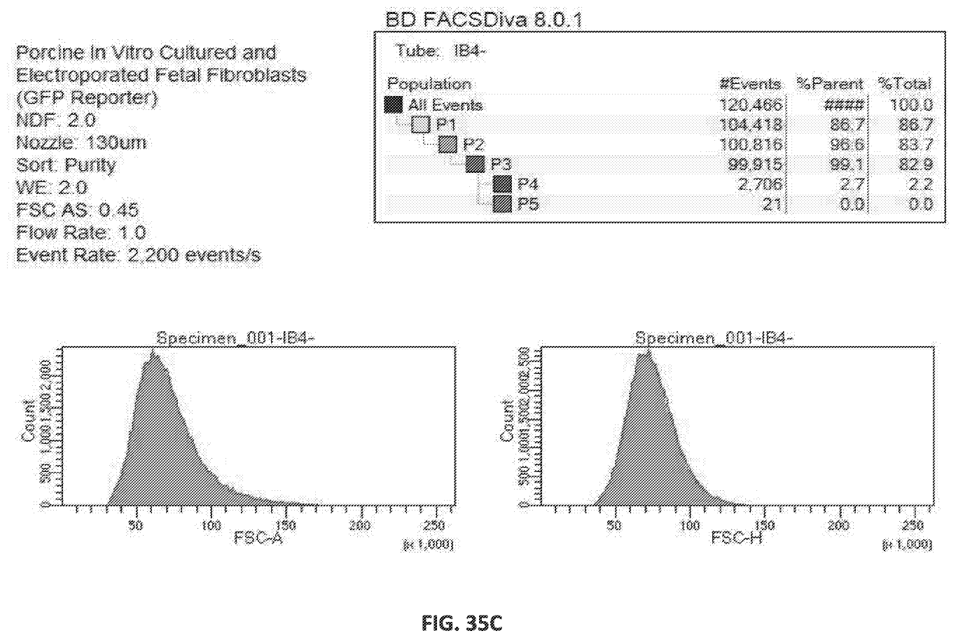

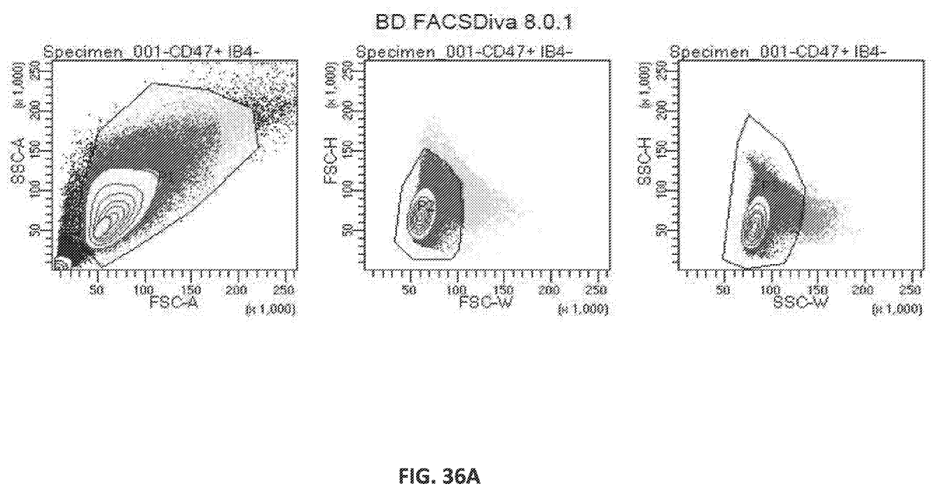

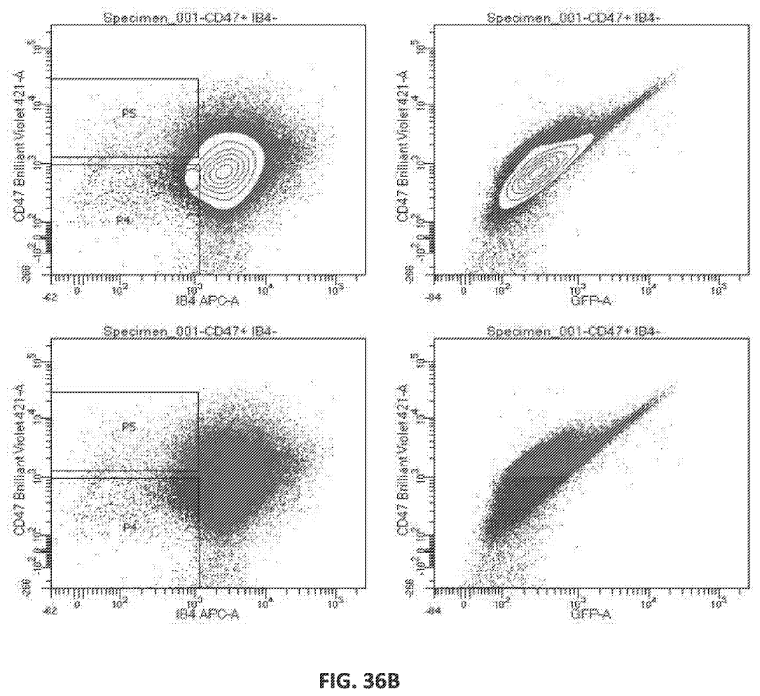

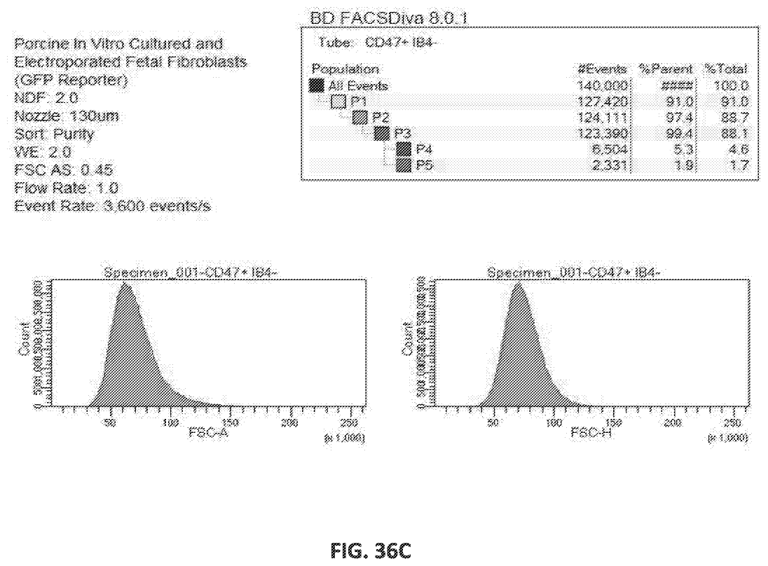

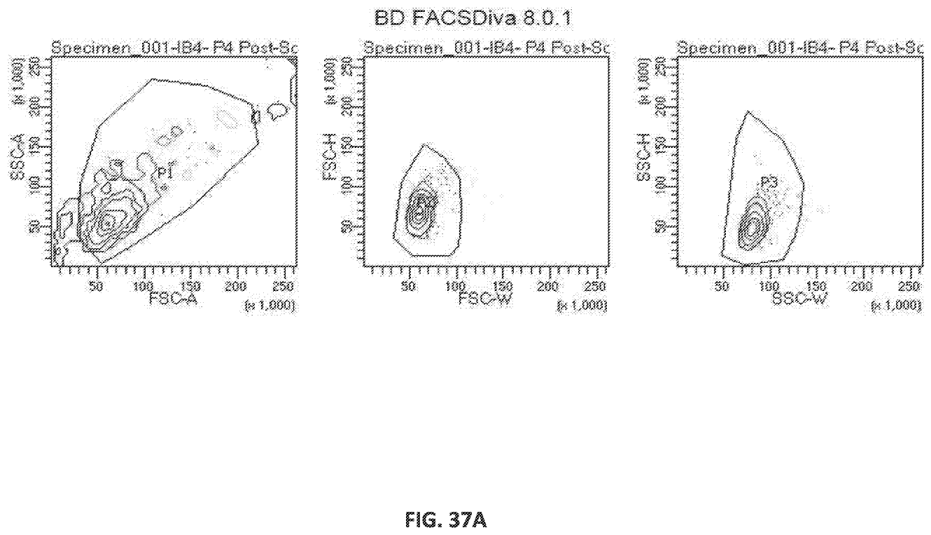

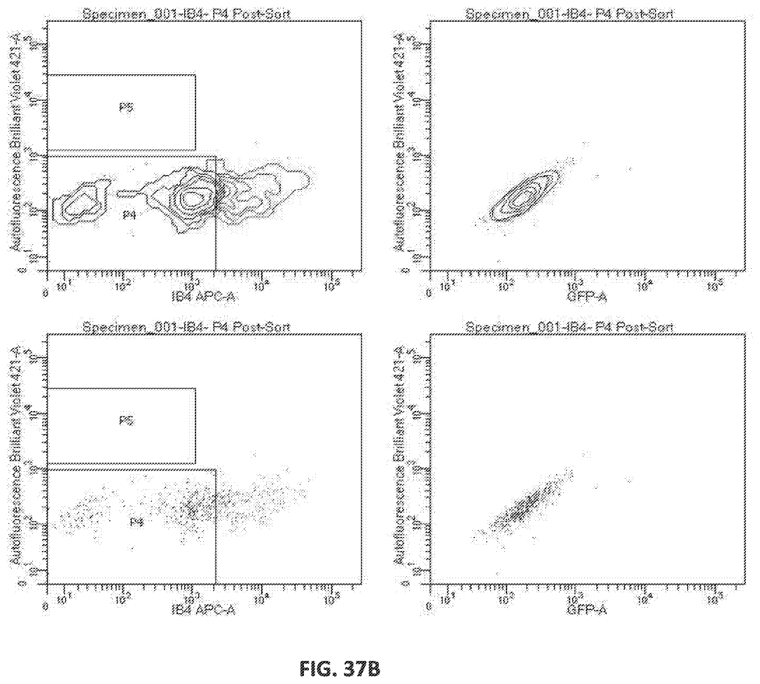

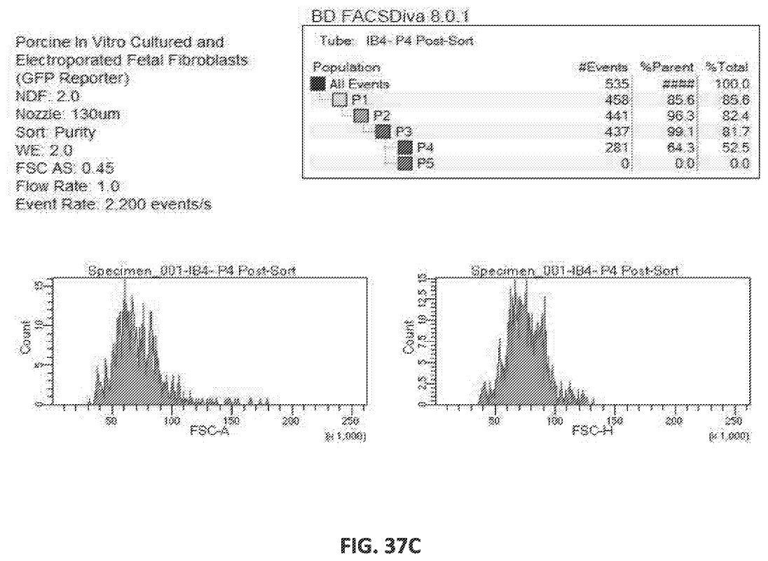

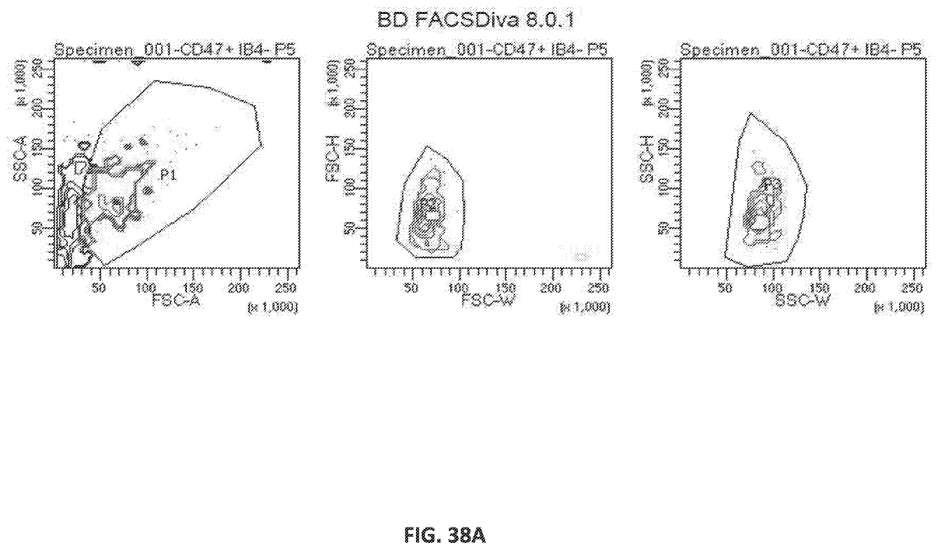

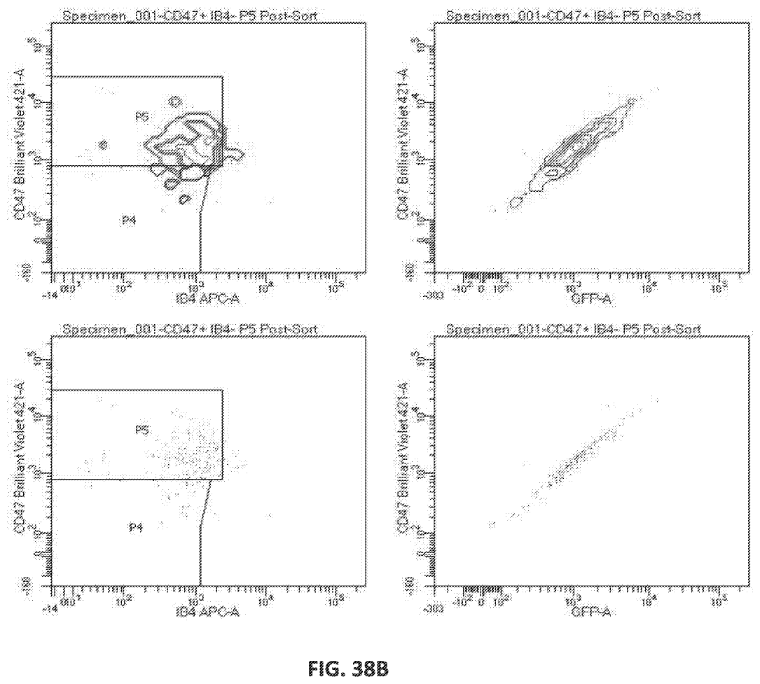

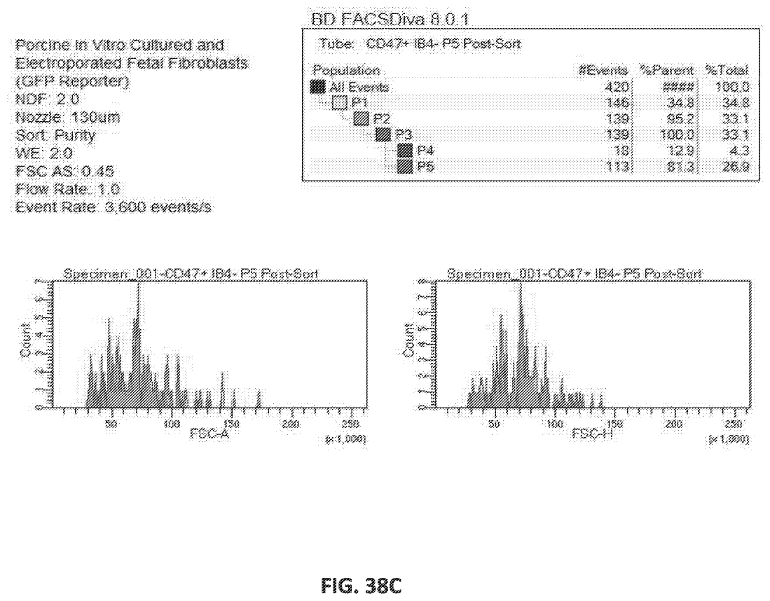

The phenotypes of the cells were examined by cell sorting. Gal antigen was detected by IB4-APC staining. CD47 was detected by CD47-Brilliant Violet 421-A. The cells sorting results were shown in FIGS. 33A-33C (unstained), FIGS. 34A-34C (px330), FIGS. 35A-35C (IB4), and FIGS. 36A-36C (CD47/IB4). Cells with GGTA1 knocked out and cells with CD47 knocked in/GGTA1 knocked out were sorted and purified for somatic cell nuclear transfer. The cell sorting results for the sorted cells were shown in FIGS. 37A-37C (IB4) and FIGS. 38A-38C (CD47/IB4).

### Example 10: The Effect of MHC Class I Deficient Porcine Fibroblast Cells (Fibroblast) on Immune Activation of Human Lymphocytes

A. Proliferation (CFSE): SLA-I/Gal-2 Knockout

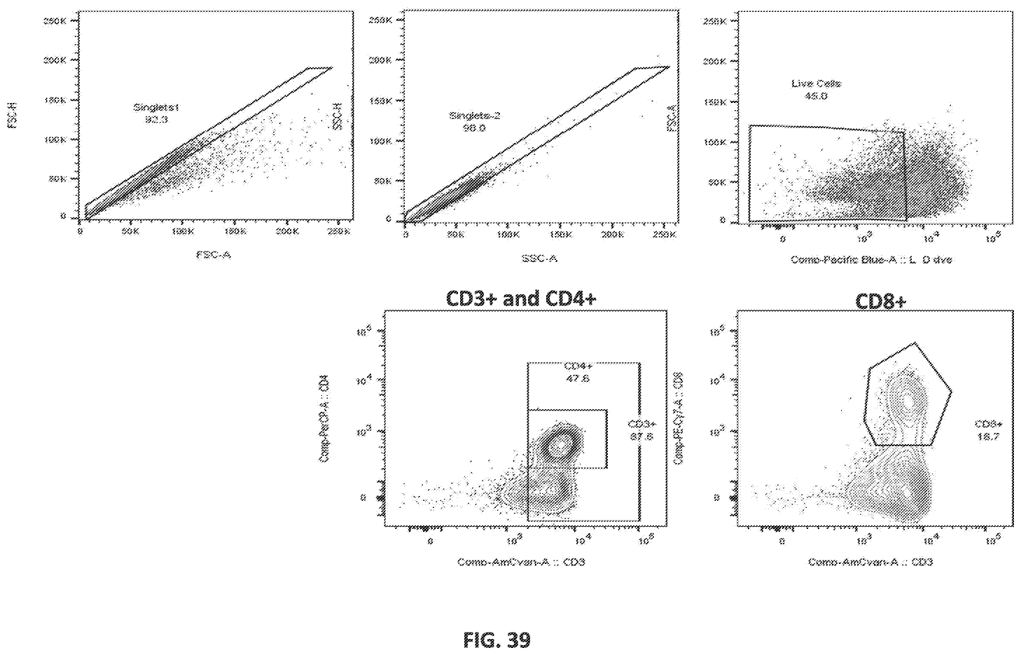

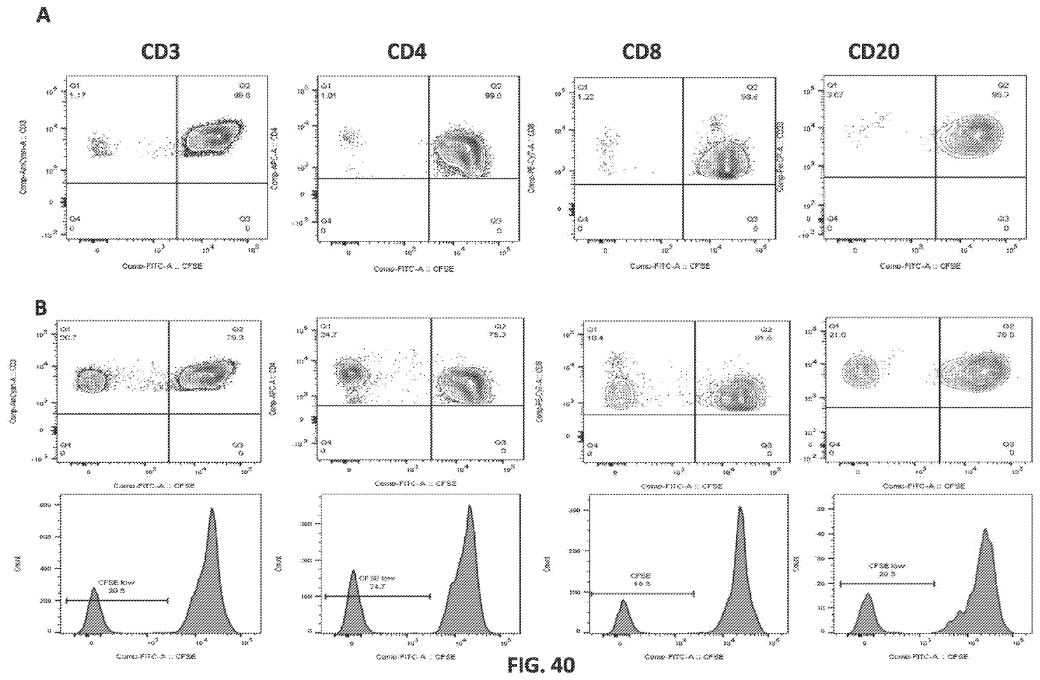

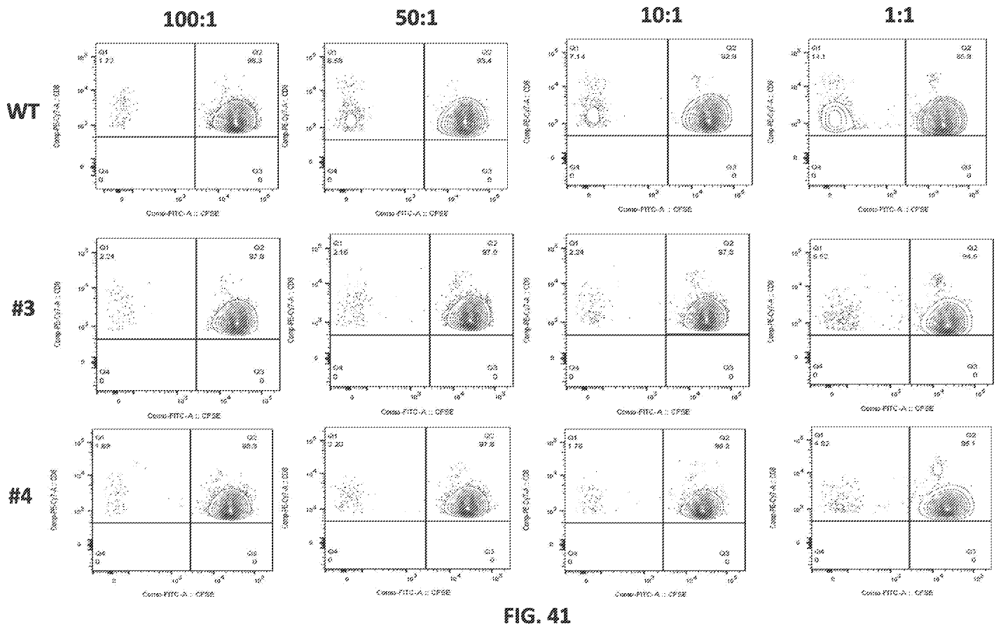

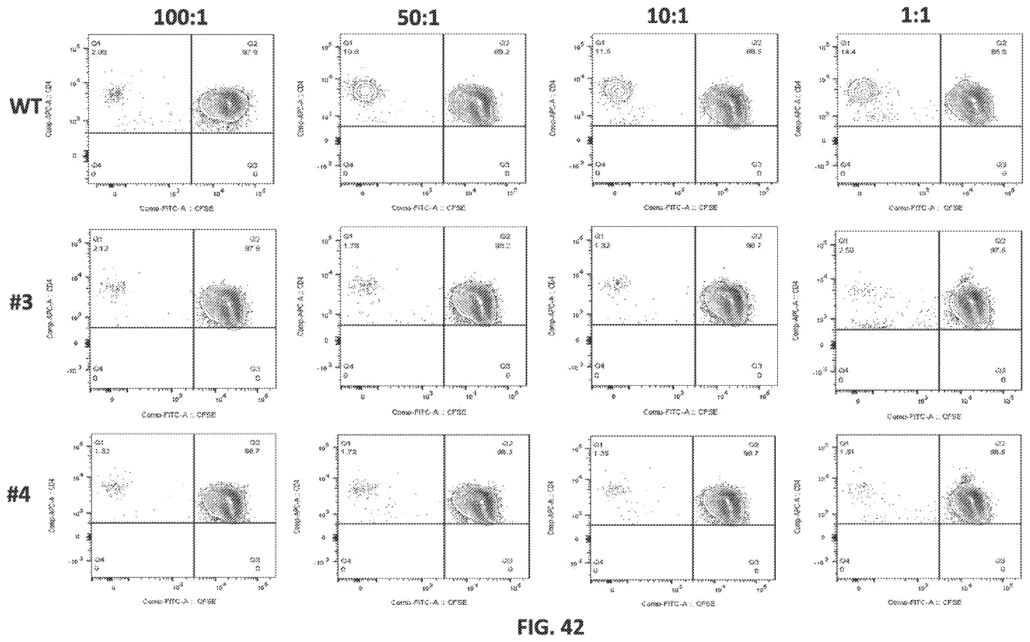

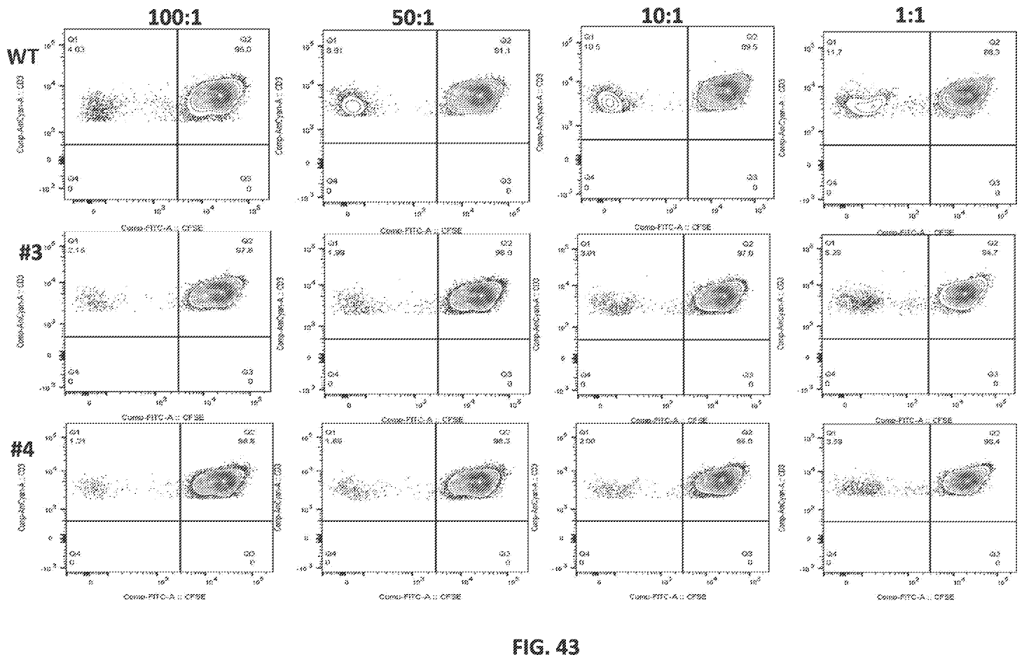

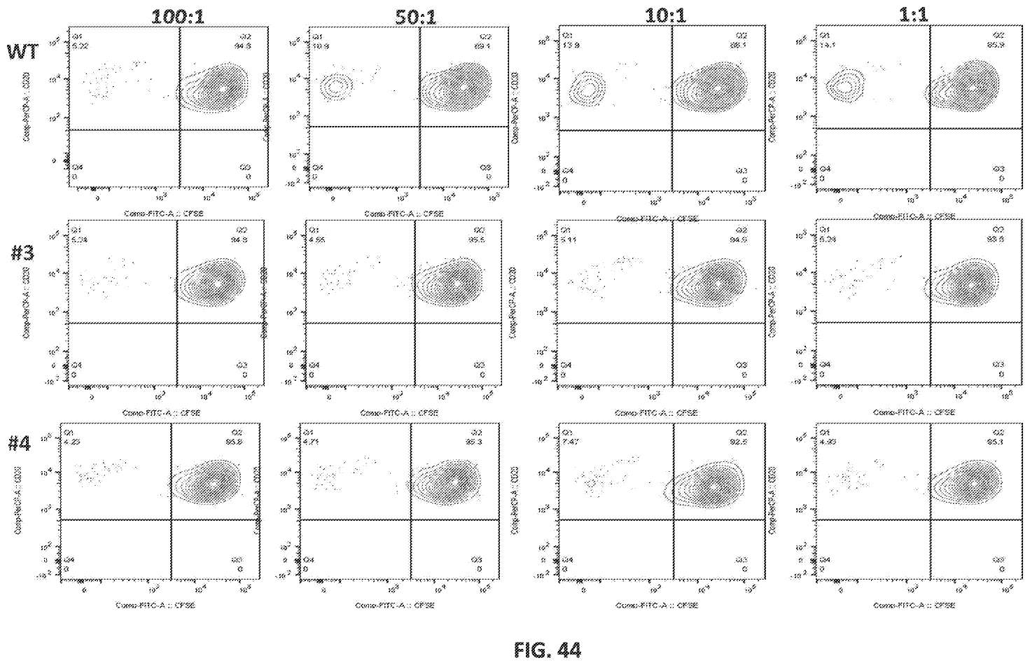

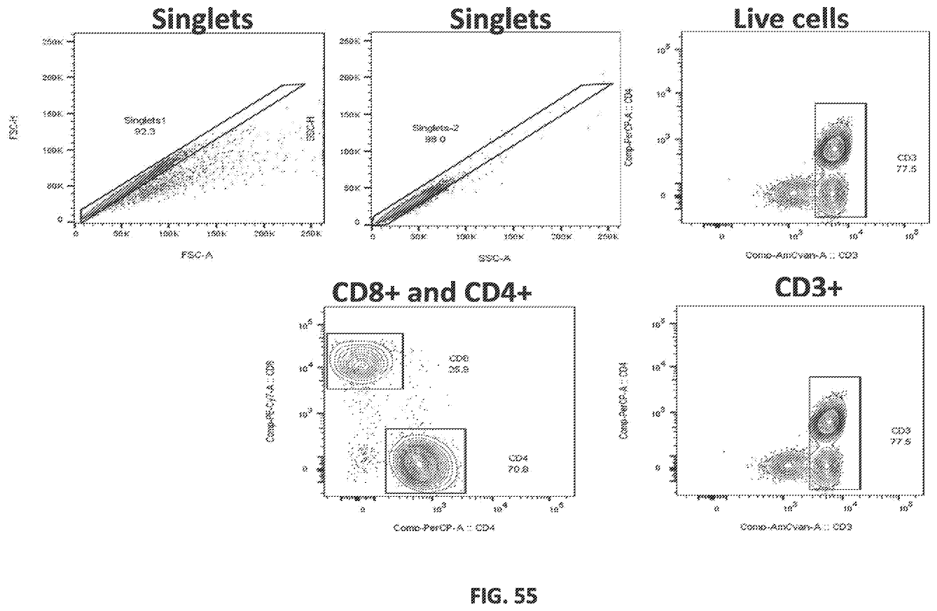

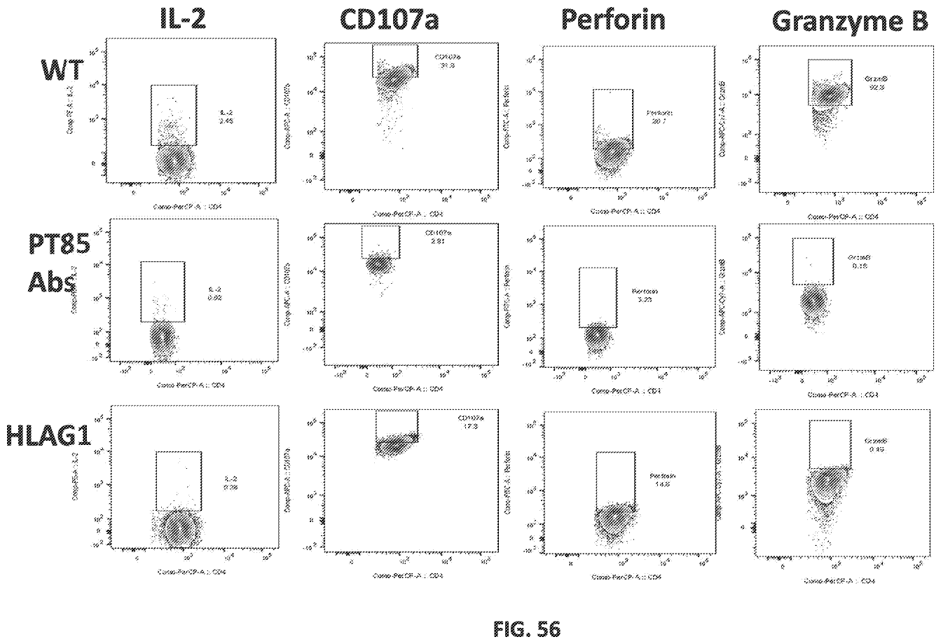

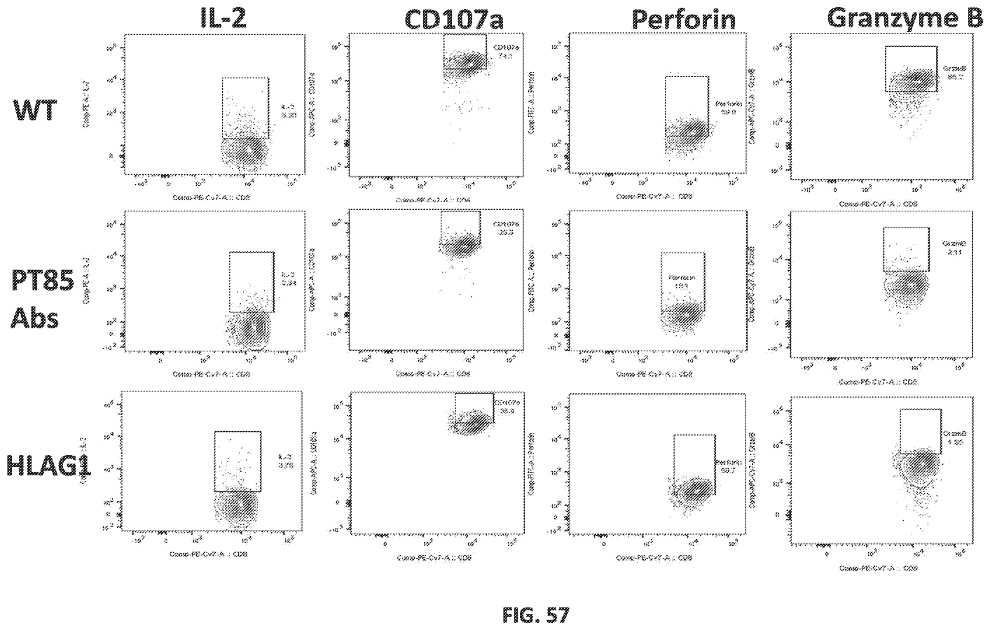

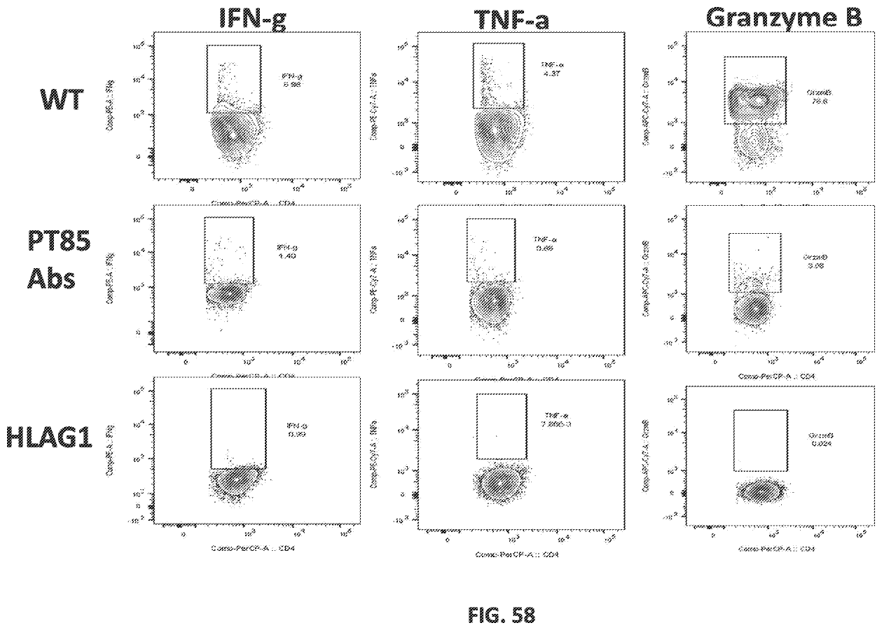

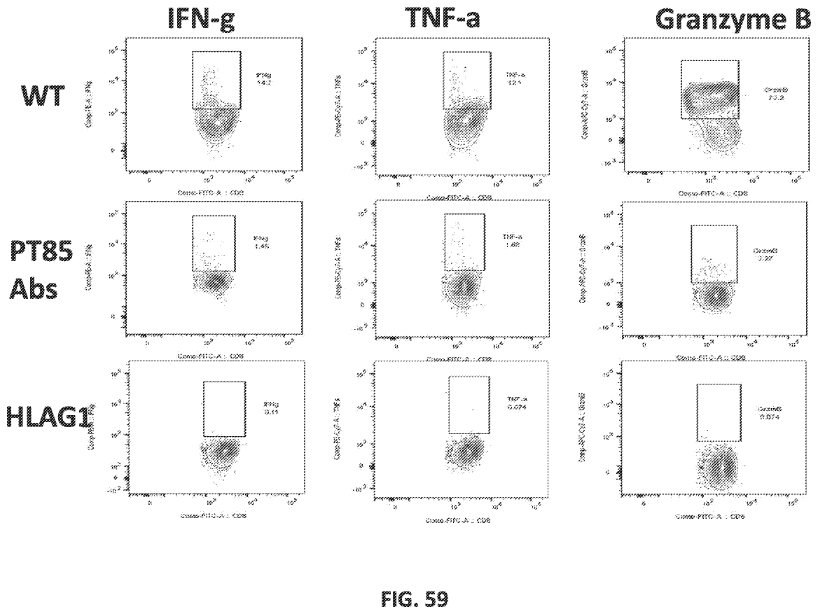

One strategy to determine the human immune response to xenotransplantation can be the co-culture of genetically modified, MHC class I deficient porcine fibroblast cells, with human PBMCs. Mixed lymphocyte reaction co-cultures were carried out in flat-bottom, 96-well plates. Human CFSE labeled (2.50 μM/ml) PBMCs, were used as responders at 1-2×105 cells/well/200 ul. Porcine fibroblast cells at 1000 to 1×10⁵ cells/well (with or without SLA-I/Gal-2 knockout) were used as stimulators at stimulator-responder ratios of 100:1, 50:1, 10:1 and 1:10. MLR co-cultures were carried out for 24 hrs for cytokines (11-2, TNF-a and IFN-g) effector molecules (Perforin, Granzyme B LAMP-1/CD107a) expression and 5-6 days for measurement of T and B cells proliferation. FIG. 39 and FIG. 55 show the gating strategy used to analyze proliferation data. Results of one human donor are shown in FIG. 40 and FIG. 41-FIG. 44. Results of additional donors are shown in FIG. 56 to FIG. 59.

B. Proliferation (CFSE): NLRC5-6/Gal-2-2 Construct: SLA-I/Gal-2Knockdown; NLRC5-6/Gal-2 Construct and GGTA1-1/Gal2-2 Construct SLA-I/Gal-2Knockdown.

Human PBMCs: Pre labeled with CFSE, were cultured with following: Control: Porcine Fibroblast: Wild Type; Condition#3 MLF cells with NLRC5-6/Gal-2-2construct: SLA-I/Gal-2knockdown; Condition#4 MLF cells with NLRC5-6/Gal-2 construct and GGTA1-1/Gal2-2 construct SLA-I/Gal-2knockdown; Culture cell density: MLF cells=4×10\hat{ }4 cells/ml; Human PBMCs=1×10\hat{ }6 cells/ml; Cells density of MLR culture: 2×10\hat{ }5 to 1.4×10\hat{ }5 cells/200 ul/well in 96 well plate flat bottom in duplicate or triplicates (Table 13).

TABLE 13
Testing plate configuration

| | | | | | RED LASER | | | |
|---|---|---|---|---|---|---|---|---|
| | | | | | (633/640 nm) | | | |
| | BLUE LASER (488 nm) | | | | | FL-6 | VIOLET LASER (405 nm) | |
| | FL-1 | | FL-3 | | FL-5 | 730/45 | FL-7 | FL-8 |
| | 530/30 | FL-2 | 670/14 | FL-4 | 660/20 | 780/60 | 450/40 | 525/50 |
| | FITC/AF488 | 575/26 | 695/40 | 780/60 | APC | APC-Cy7 | PacB(401/452) | BDHV500 |
| | BDHB- | PE | PECy5 PerCP | PECy7 | AF647 | AF700 | BV421/450 | AmCyan/PacO |
| | Blue515 | PI | PerCP Cy5.5 | 496/785 | eF660 | APC-H7 | BDH450 | BV500/BV510 |
| BS1 | CFSE | x | CD20 PerCP, | CD8 PECy7 | CD4 APC | X | L/D dye | CD3 V500 |
| | | | 20 ul | 5 ul | 5 ul | | | 5 ul |
| IC-1 | Perforin | IL-2 | CD4 | CD8 | CD107a | Granzyme B | ″ | CD3 V500 |
| | 5 ul | 5 ul | 20 ul | 5 ul | 5 ul | 5 ul | | 5 ul |
| IC-2 | CD8 | IFN-g | CD4 | TNF-a | CD56 | Granzyme B | ″ | CD3 V500 |
| | 20 ul | 5 ul | 20 ul | 5 ul | 5 ul | CD20 | | 5 ul |
| | | | | | | 10 ul | | |
| FMO | CD8 | | CD4 | | CD56 | Granzyme B | ″ | CD3 V500 |
| FMD | 20 ul | | 20 ul | | 5 ul | CD20 | | 5 ul |
| | | | | | | 10 ul | | |

C. Intracellular Cytokine Staining (ICCS)

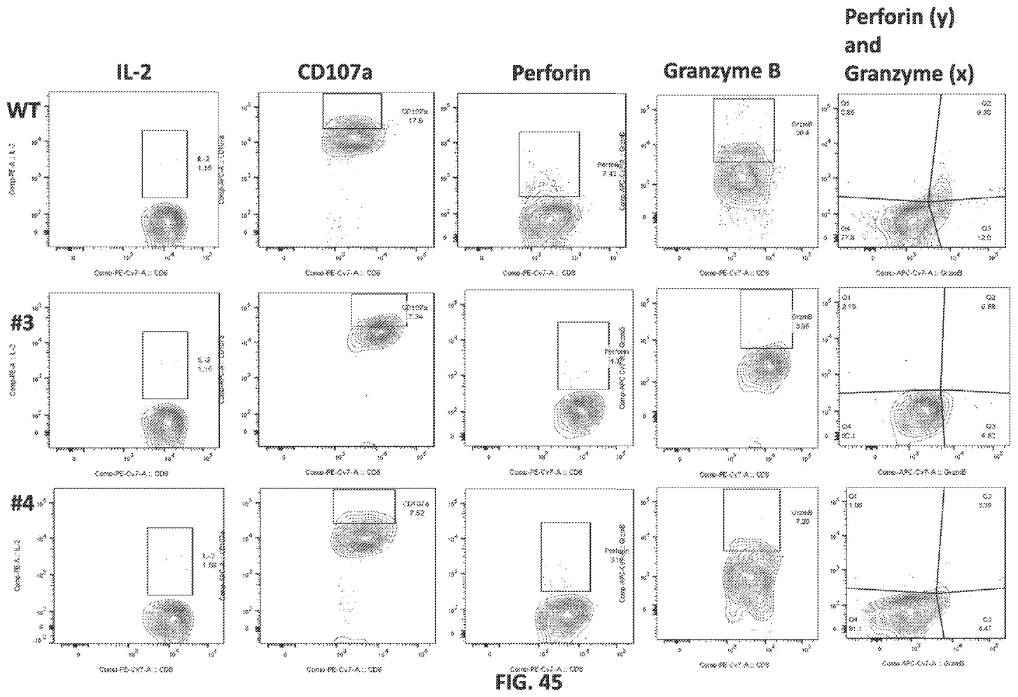

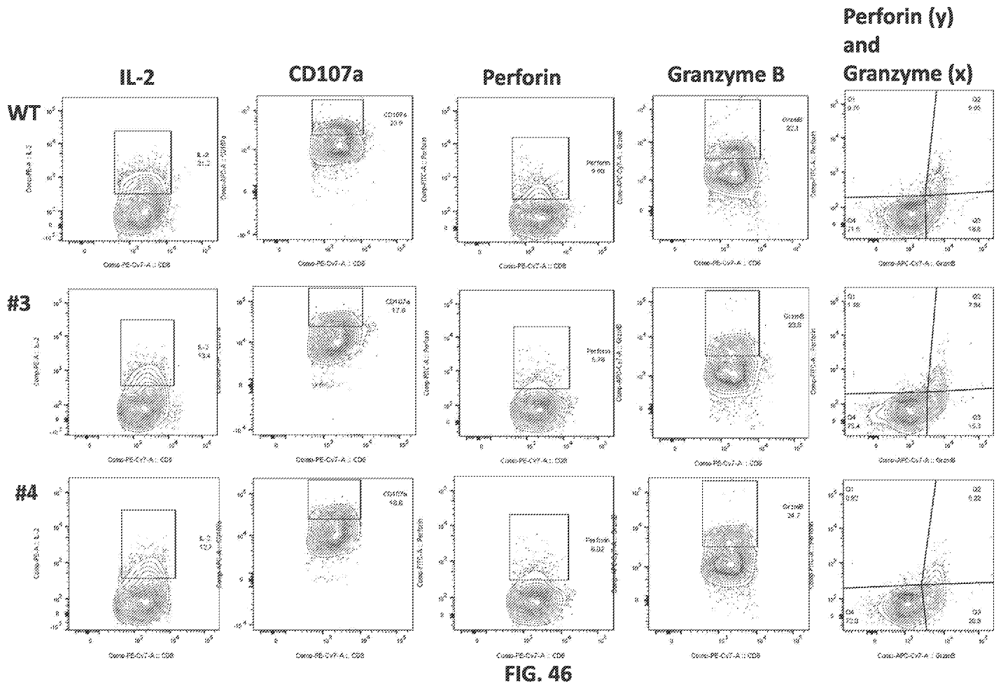

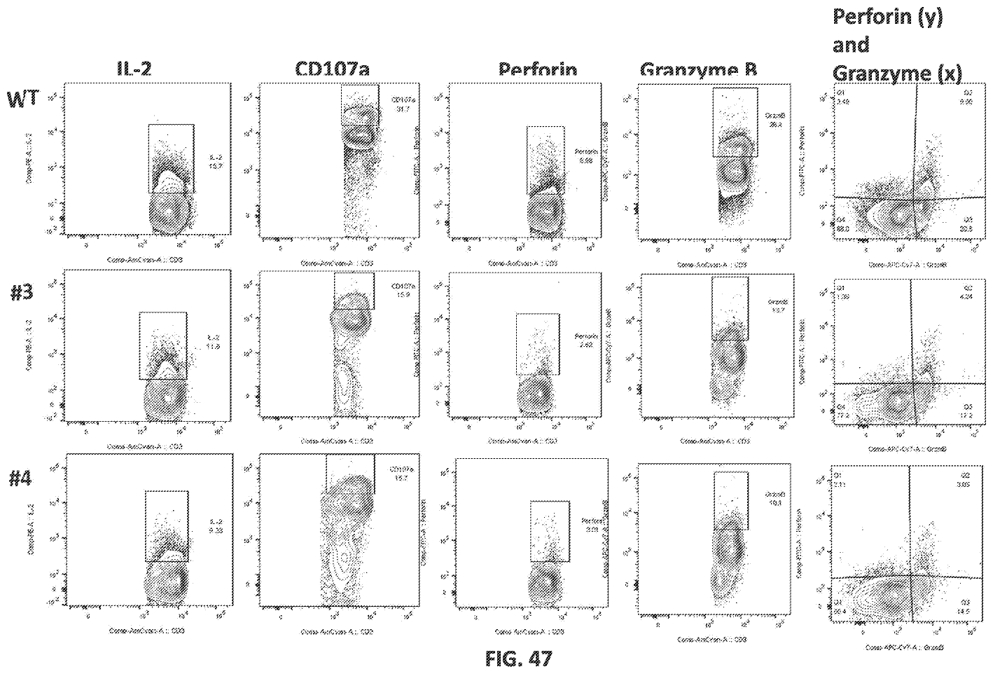

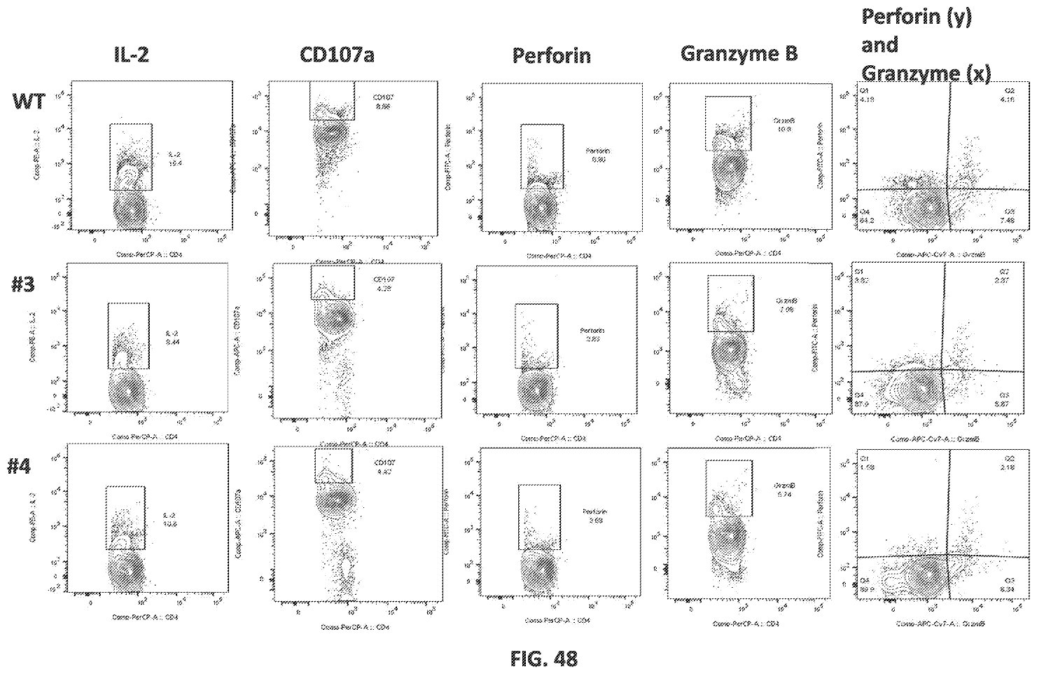

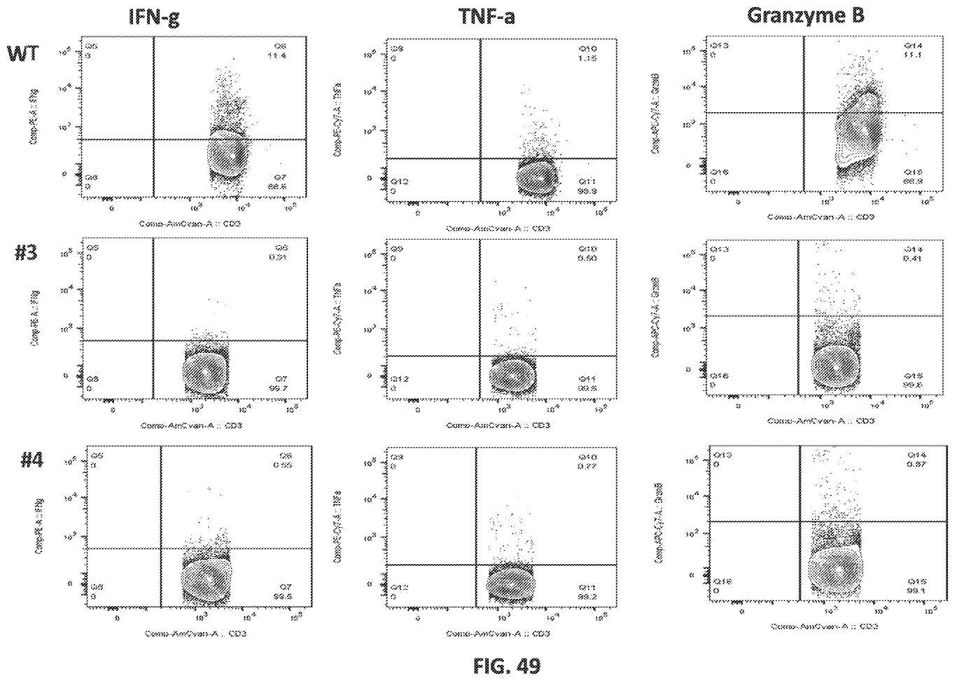

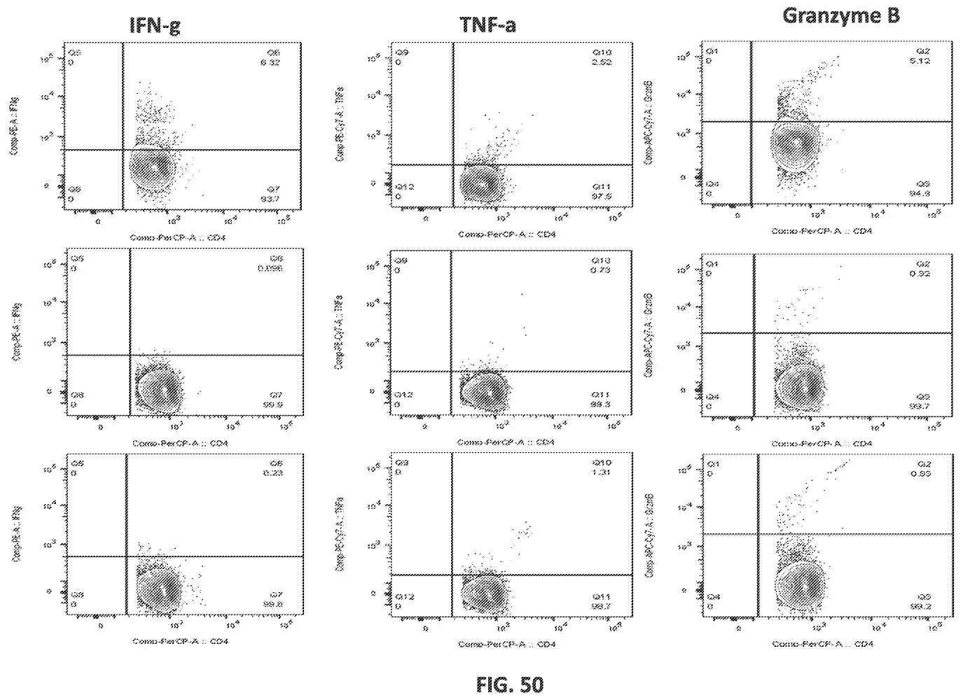

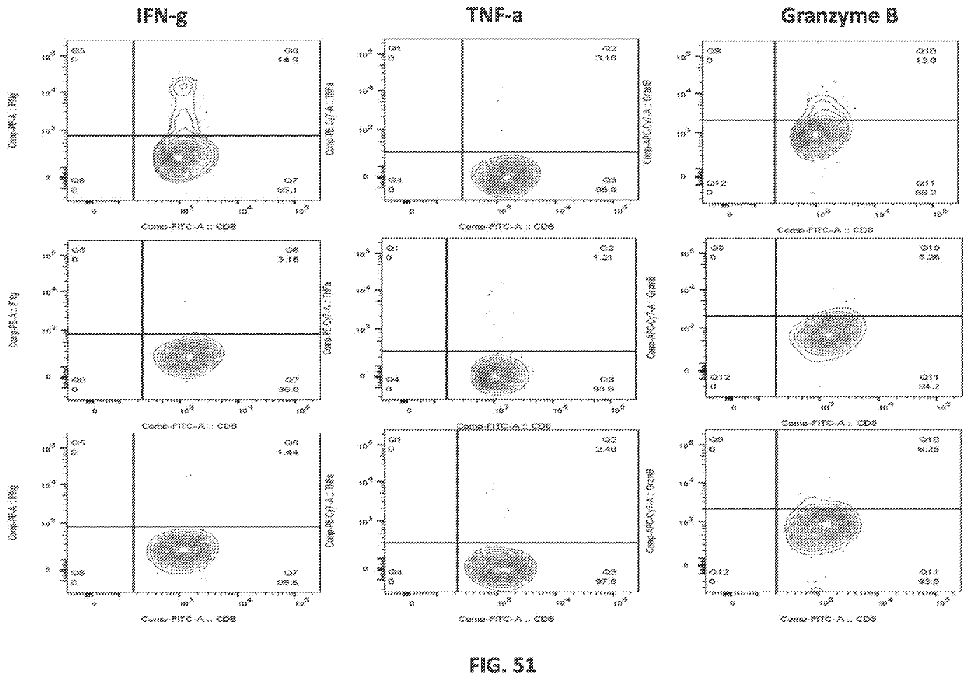

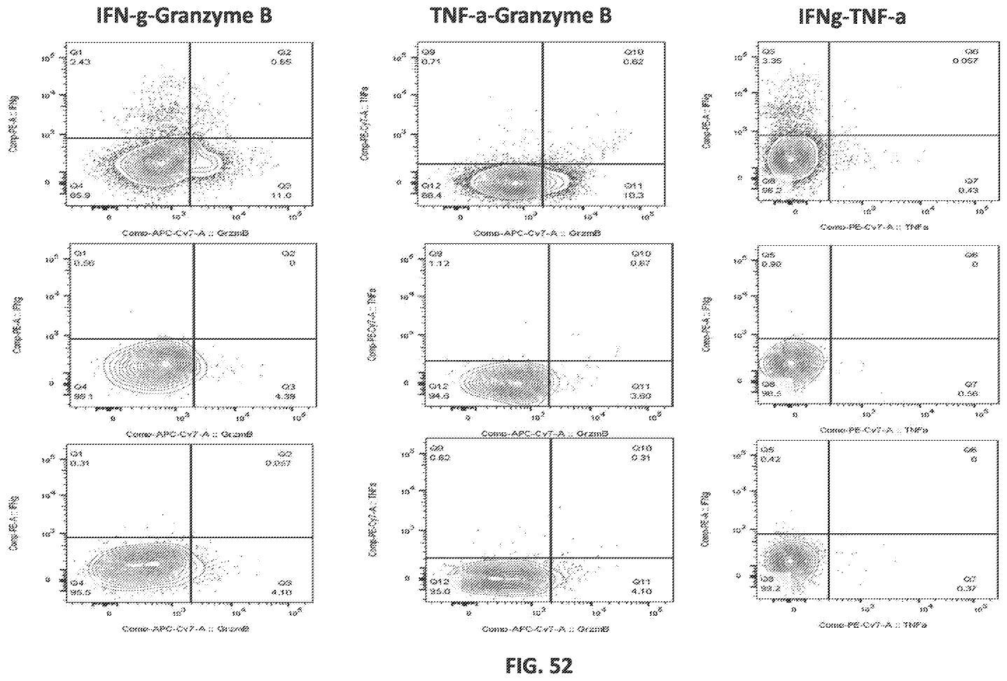

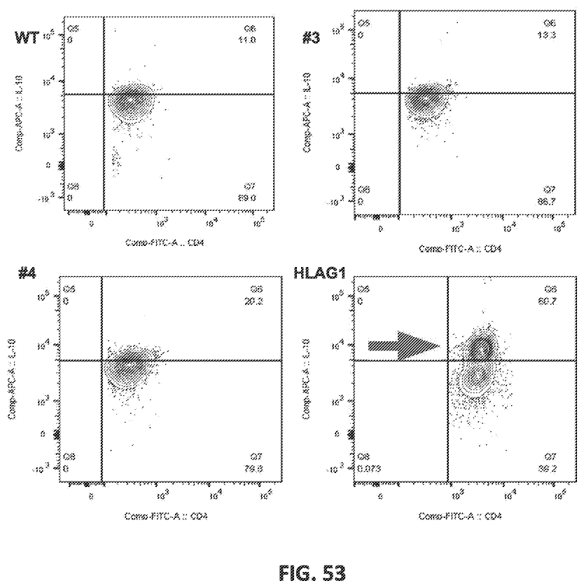

In a parallel experiment (Table 14) total PBMCs cells were stimulated with and without PHA (2 ug/ml) as positive and unstimulated control respectively. Cultured cells were washed and stained with anti-CD3, anti-CD4 and anti-CD8 followed by formaldehyde fixation and washing and intracellular staining with anti-perforin, granzyme B, IL-2, TNF-a and IFN-g (FIG. 45 to FIG. 52). BD FACS Canto II flow, were used to assessed the proliferative capacity of CD8 and CD4 T cells in response to SLA-I knockout porcine fibroblast (F3) compared to unmodified porcine fibroblast cells. Data were analyzed using FACS diva/Flow Jo software (Tri star, San Diego, Calif., USA), and percentage CFSE dim/low was determined on pre gated CD8 T cells and CD4 T cells.

TABLE 14
ICCS Experimental Configuration

| | | PBMCs + | Ratios of Human PBMCs vs FC | | | |
|---|---|---|---|---|---|---|
| | PBMCs alone | PHA | 100:1 | 50:1 | 10:1 | 1;1 |
| WT | 2 × 10\hat{ }5 | 2 × 10\hat{ }5 | 1 × 10\hat{ }5 + | 1 × 10\hat{ }5 + | 1 × 10\hat{ }5 + | 1 × 10\hat{ }5 + |
| | | | 1000MLF | 2000MLF | 10,000MLF | 1 × 10\hat{ }5 MLF |
| #3 | 2 × 10\hat{ }5 | 2 × 10\hat{ }5 | 1 × 10\hat{ }5 + | 1 × 10\hat{ }5 + | 1 × 10\hat{ }5 + | 1 × 10\hat{ }5 + |
| | | | 1000MLF | 2000MLF | 10,000MLF | 1 × 10\hat{ }5 MLF |
| #4 | 2 × 10\hat{ }5 | 2 × 10\hat{ }5 | 1 × 10\hat{ }5 + | 1 × 10\hat{ }5 + | 1 × 10\hat{ }5 + | 1 × 10\hat{ }5 + |
| | | | 1000MLF | 2000MLF | 10,000MLF | 1 × 10\hat{ }5 MLF |

### Example 11: Methodology for Mixed Cell Cultures Including PT85 Antibody

Mixed lymphocyte reaction co-cultures were carried out in flat-bottom, 96-well plates. Human CFSE labeled (2.50 μM/ml) PBMCs, were used as responders at 1-2×10⁵ cells/well/200 ul. Porcine fibroblast cells/WT or HLA-G transduced at 2000 to 1×10⁵ cells/well (with or without PT85 Ab/blocking Abs, 10 ug/ml) were used as stimulators at stimulator-responder ratios of 100:1, 50:1, 10:1 and 1:10. MLR co-cultures were carried out for 24 hrs for cytokines (11-2, TNF-a and IFN-g) effector molecules (Perforin, Granzyme B LAMP-1/CD107a) expression and 5-6 days for measurement of T and B cells proliferation. In another parallel experiment total PBMCs cells were stimulated with and without PHA (2 ug/ml) as positive and unstimulated control respectively. Cultured cells were washed and stained with anti-CD3, anti-CD4 and anti-CD8 followed by formaldehyde fixation and washing and intracellular staining with anti-perforin, granzyme B, IL-2, TNF-a and IFN-g. BD FACS Canto II flow, were used to assessed the proliferative capacity of CD8 and CD4 T cells in response to SLA-I knockout porcine fibroblast (F3) compared to unmodified porcine fibroblast cells. Data were analyzed using FACS diva/Flow Jo software (Tri star, San Diego, Calif., USA), (Table 15).

TABLE 15
Flow Cytometry Experimental Configuration

| | | | | | RED LASER | | | |
|---|---|---|---|---|---|---|---|---|
| | | | | | (633/640 nm) | | | |
| | BLUE LASER (488 nm) | | | | | FL-6 | VIOLET LASER (405 nm) | |
| | FL-1 | | FL-3 | | FL-5 | 730/45 | FL-7 | FL-8 |
| | 530/30 | FL-2 | 670/14 | FL-4 | 660/20 | 780/60 | 450/40 | 525/50 |
| | FITC/AF488 | 575/26 | 695/40 | 780/60 | APC | APC-Cy7 | PacB(401/452) | BDHV500 |
| | BDHB- | PE | PECy5 PerCP | PECy7 | AF647 | AF700 | BV421/450 | AmCyan/PacO |
| | Blue515 | PI | PerCP Cy5.5 | 496/785 | eF660 | APC-H7 | BDH450 | BV500/BV510 |
| BS1 | CFSE | x | CD20 PerCP, | CD8 PECy7 | CD4 APC | X | L/D dye | CD3 V500 |
| | | | 20 ul | 5 ul | 5 ul | | | 5 ul |
| IC-1 | Perforin | IL-2 | CD4 | CD8 | CD107a | Granzyme B | ″ | CD3 V500 |
| | 5 ul | 5 ul | 20 ul | 5 ul | 5 ul | 5 ul | | 5 ul |
| IC-2 | CD8 | IFN-g | CD4 | TNF-a | CD56 | Granzyme B | ″ | CD3 V500 |
| | 20 ul | 5 ul | 20 ul | 5 ul | 5 ul | CD20 | | 5 ul |
| | | | | | | 10 ul | | |
| FMO | CD8 | | CD4 | | CD56 | Granzyme B | ″ | CD3 V500 |
| FMD | 20 ul | | 20 ul | | 5 ul | CD20 | | 5 ul |
| | | | | | | 10 ul | | |

### Example 12: Blocking MHC Class 1 Molecule/TCR Interaction PT-85 Antibody

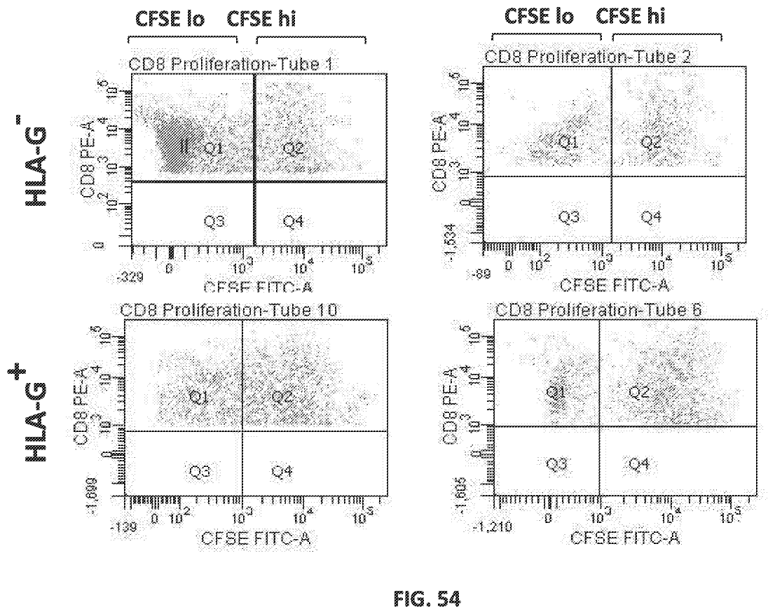

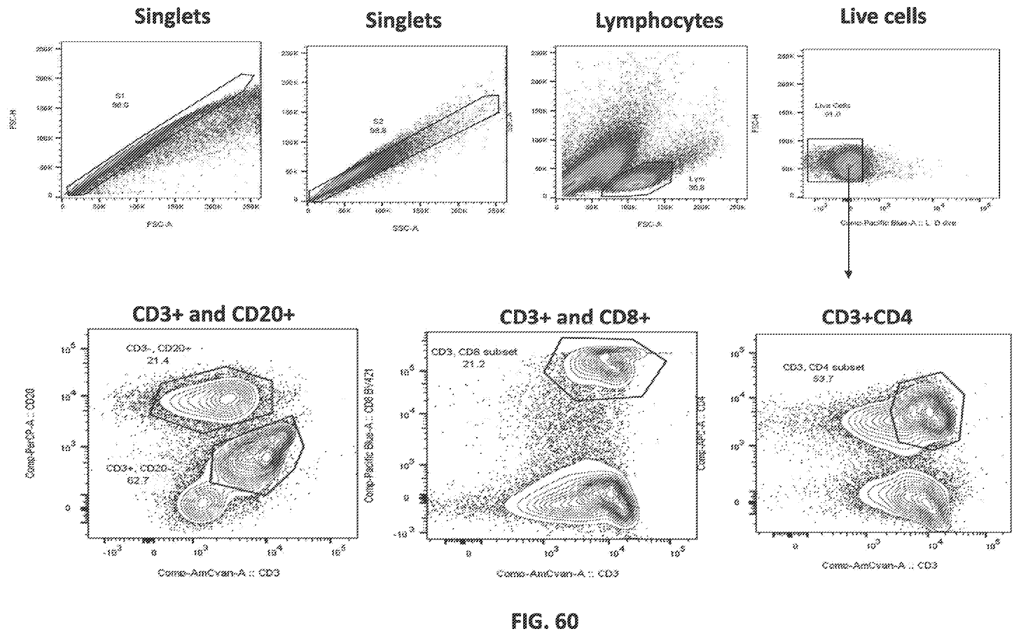

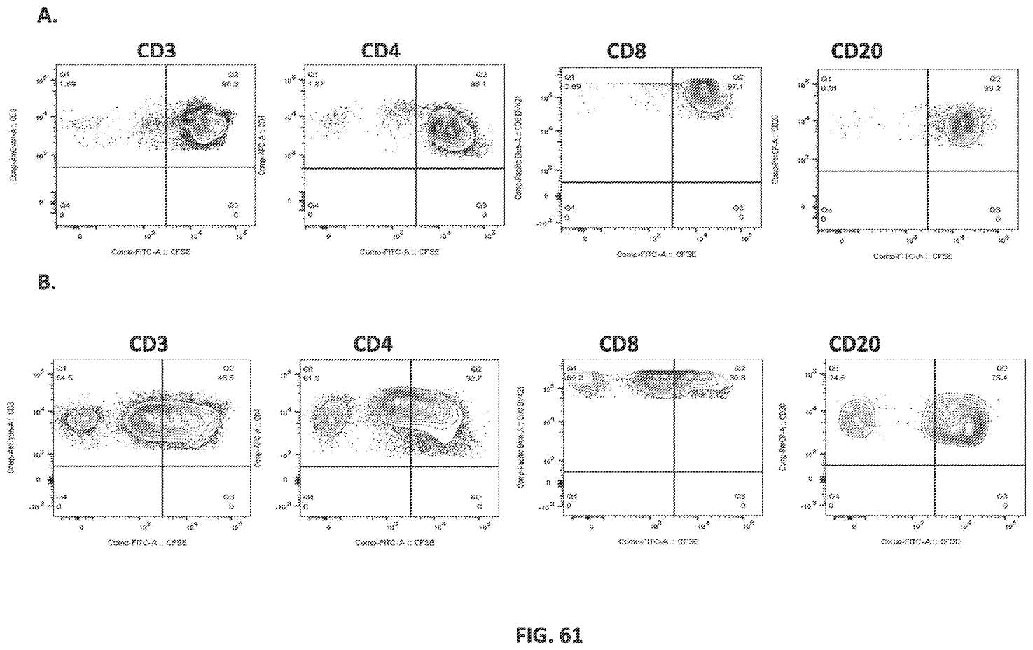

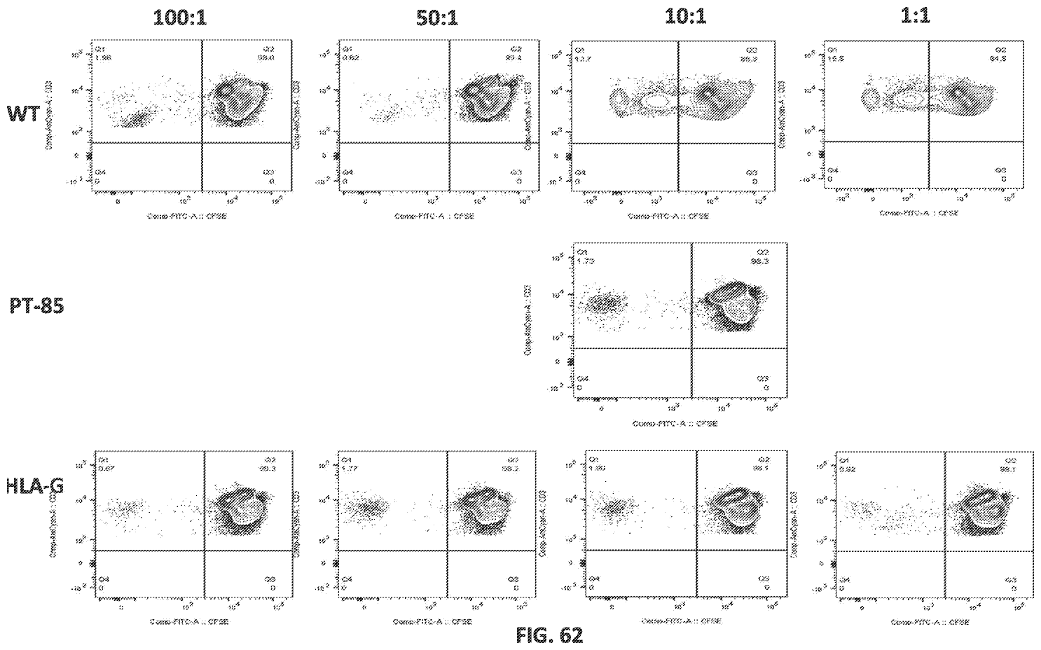

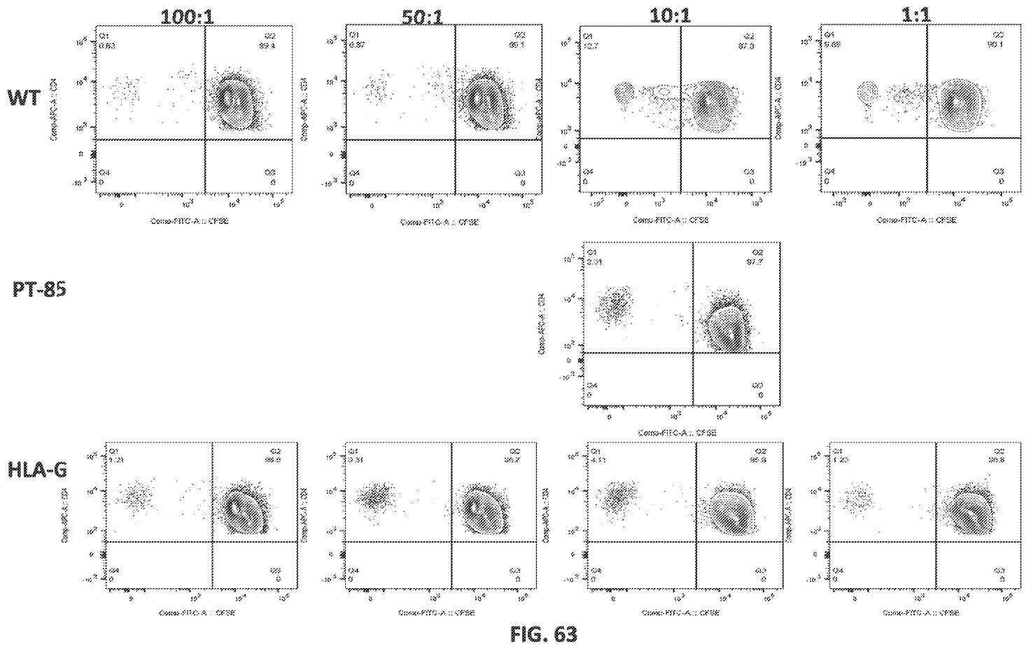

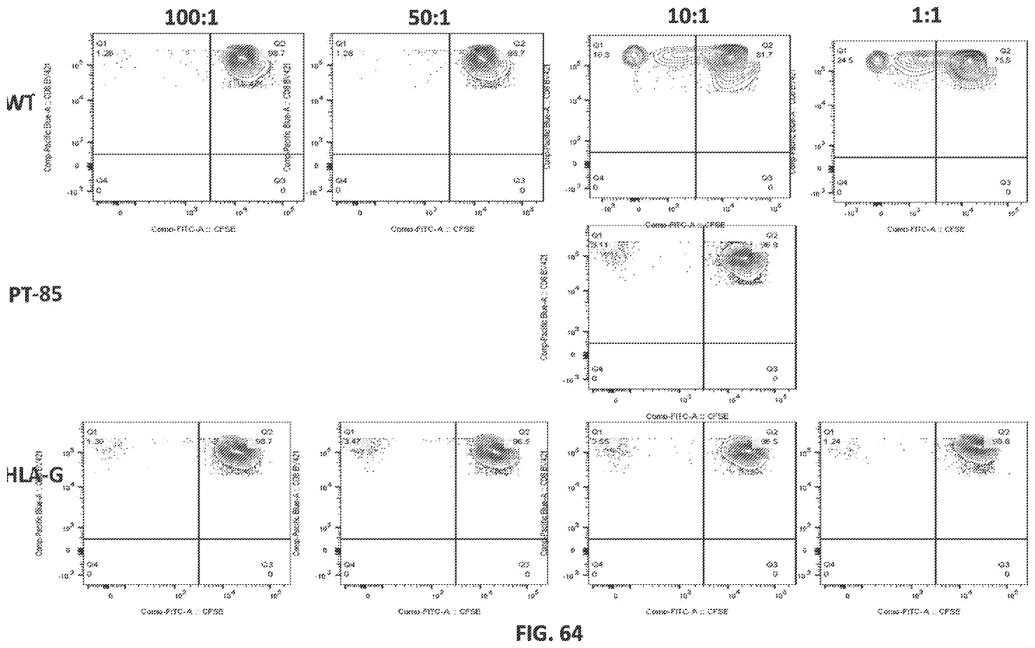

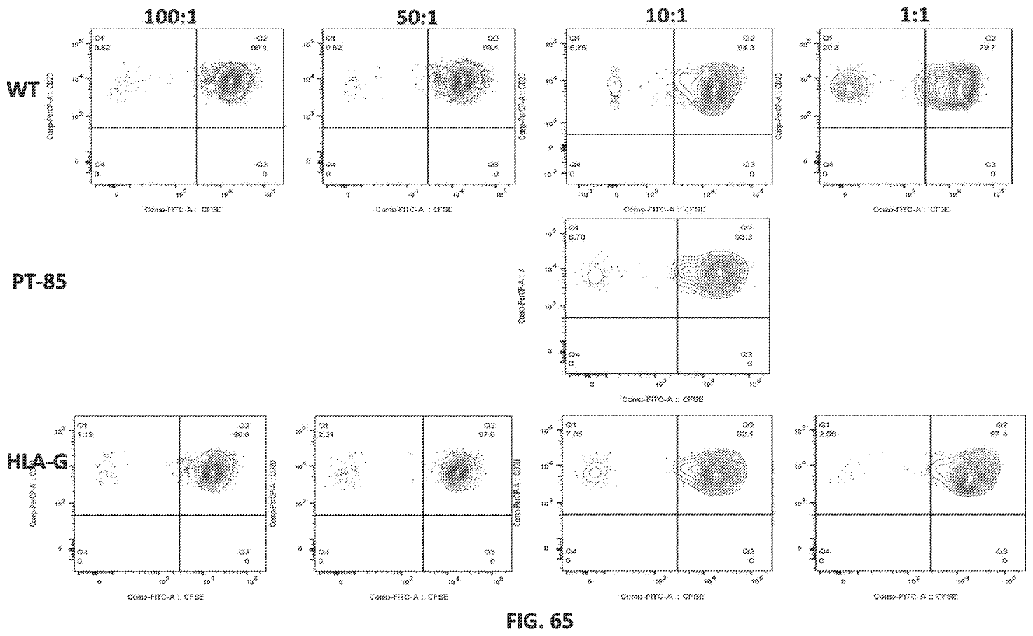

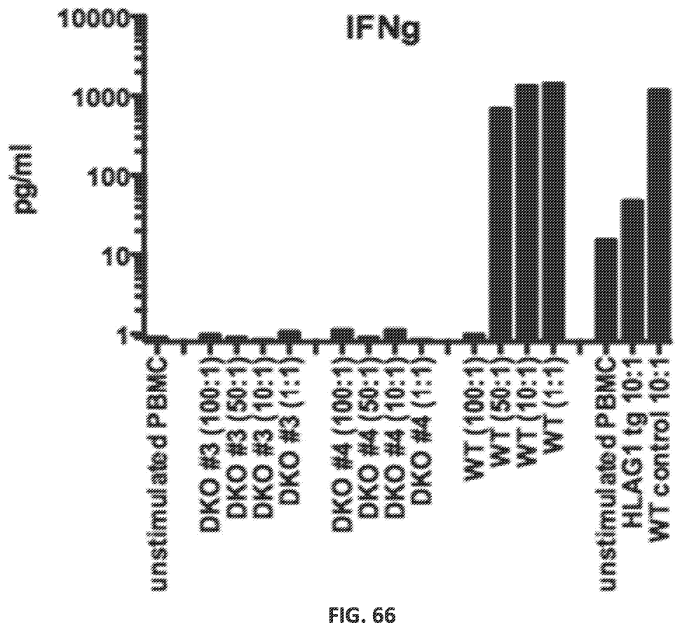

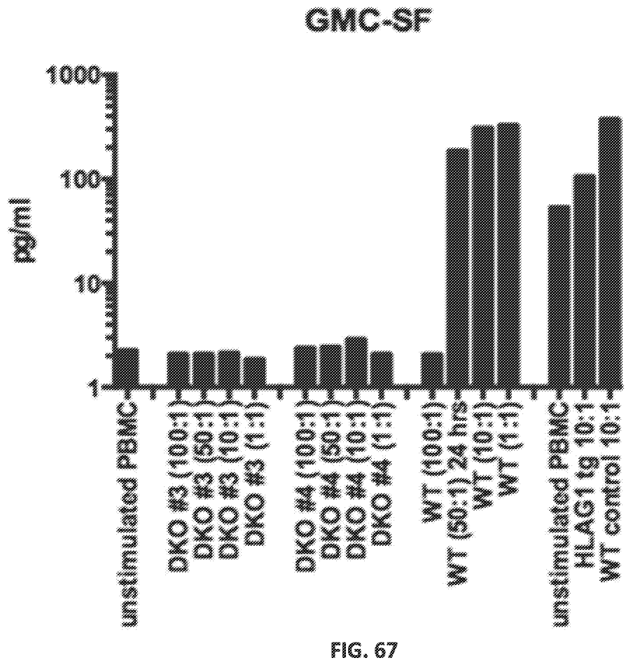

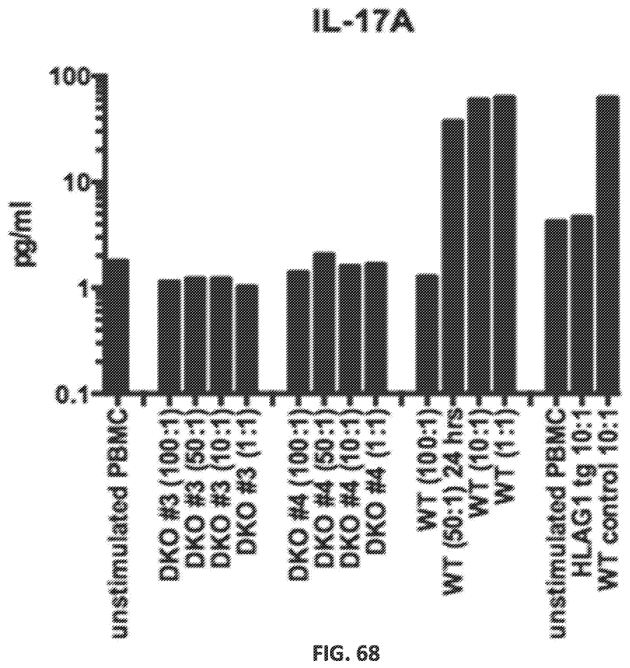

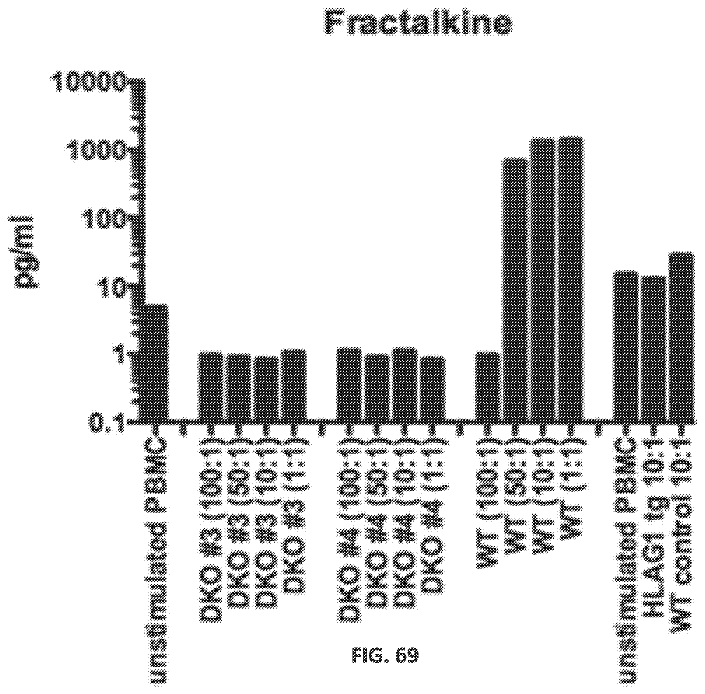

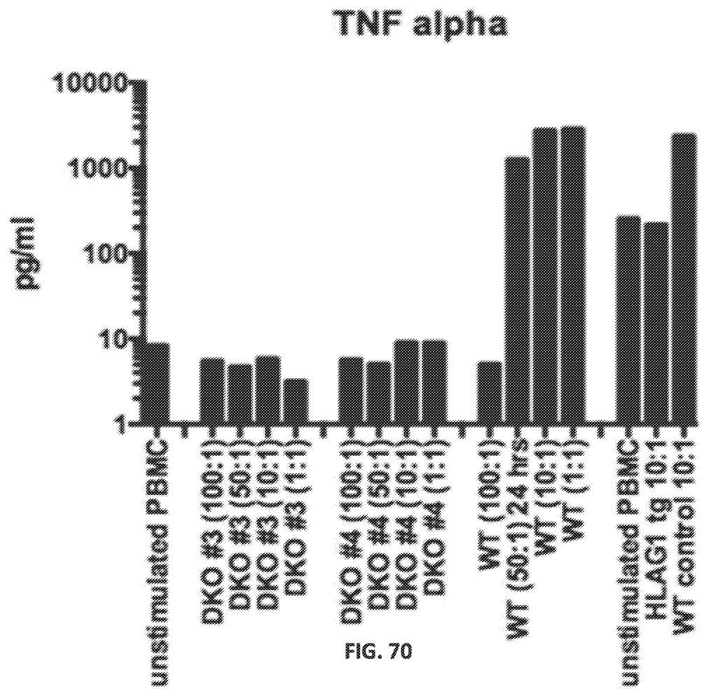

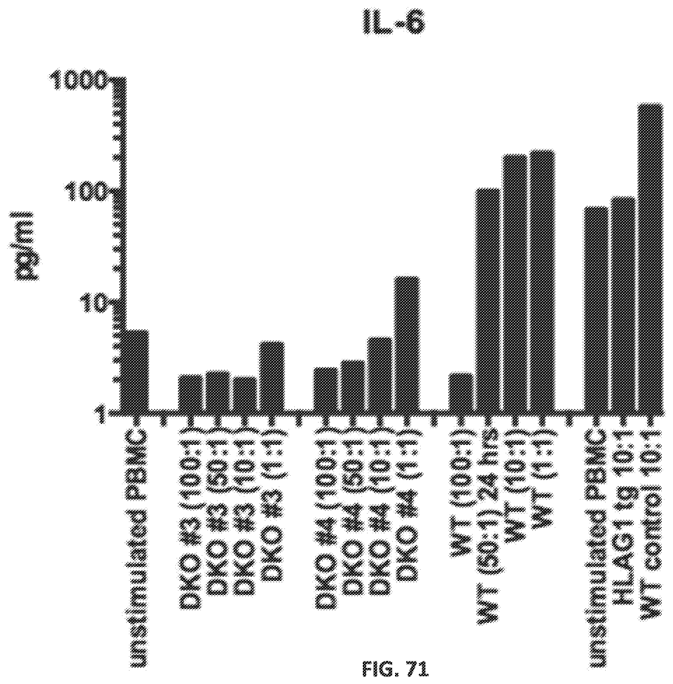

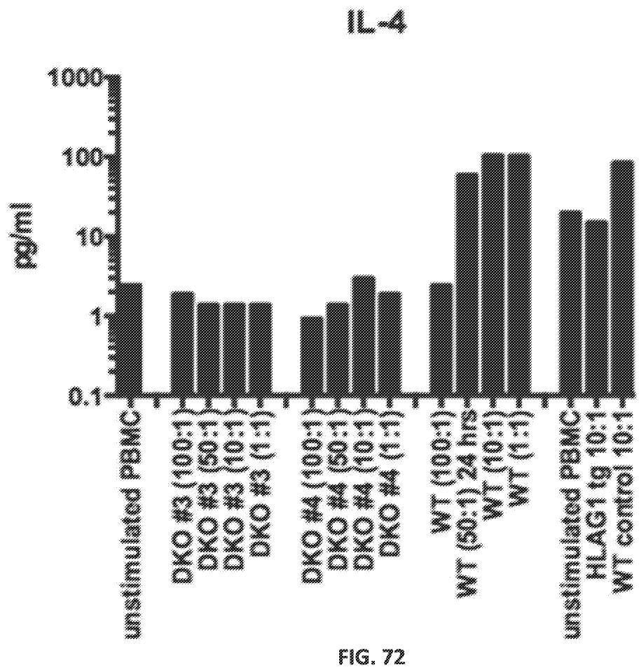

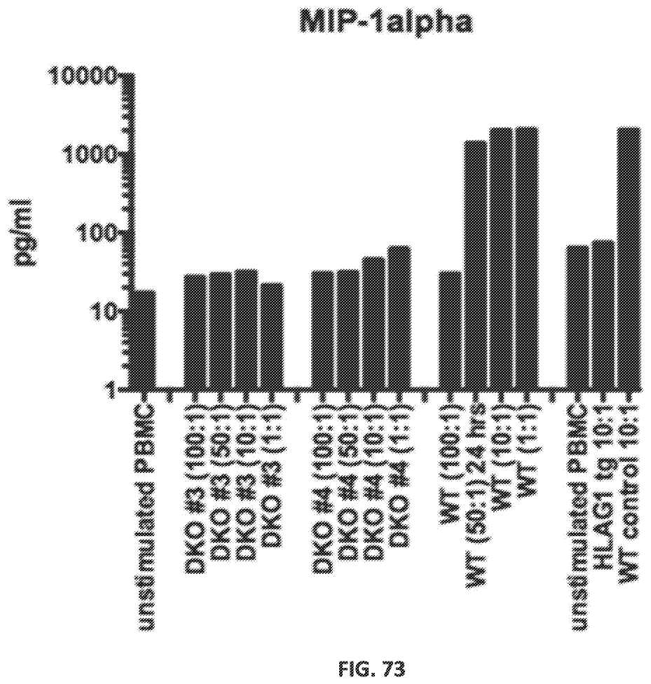

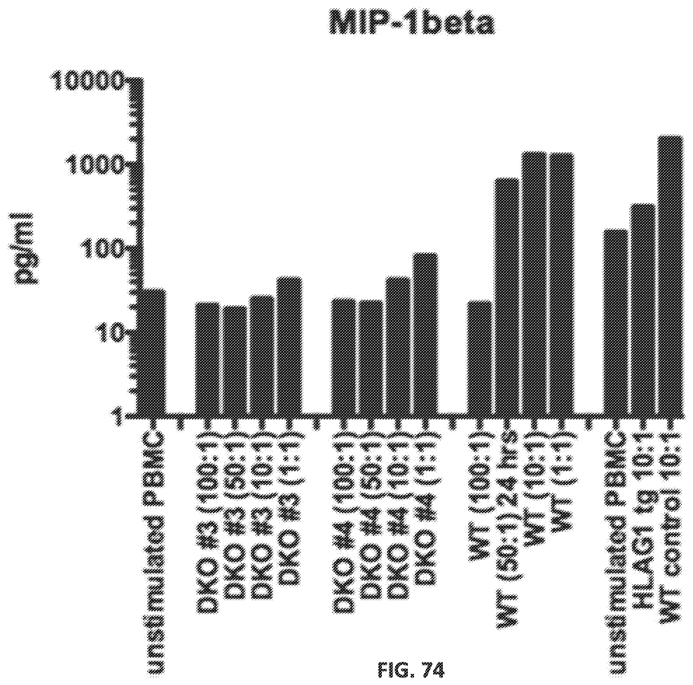

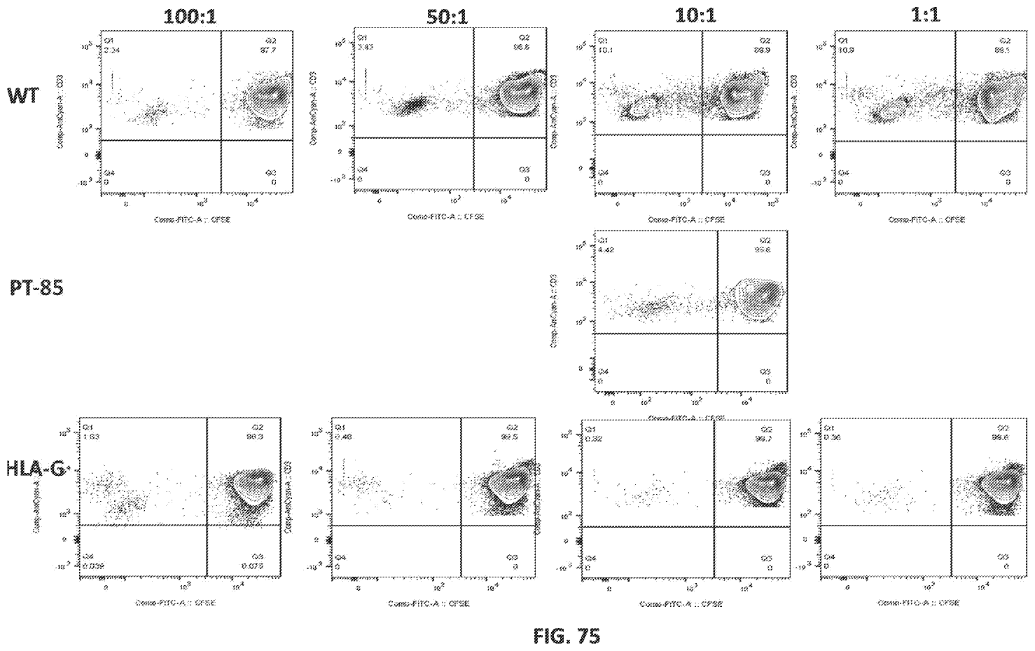

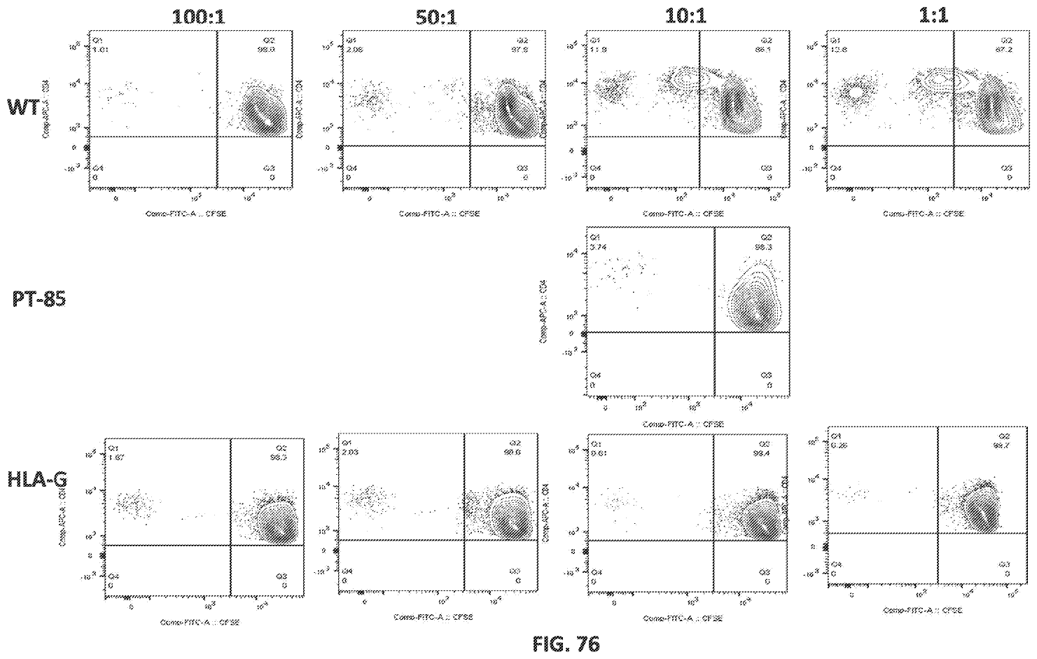

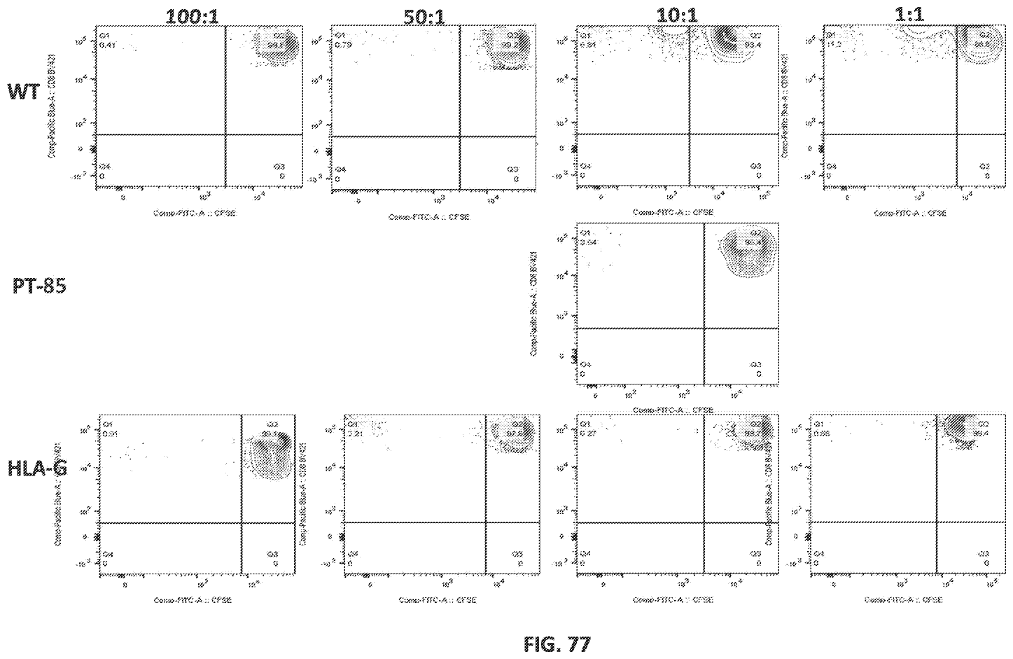

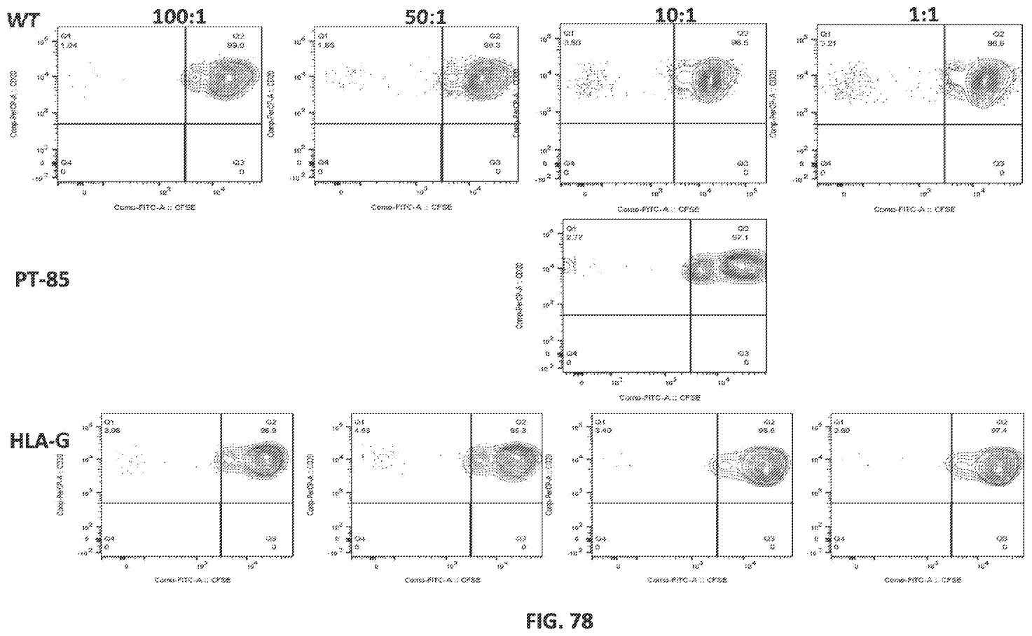

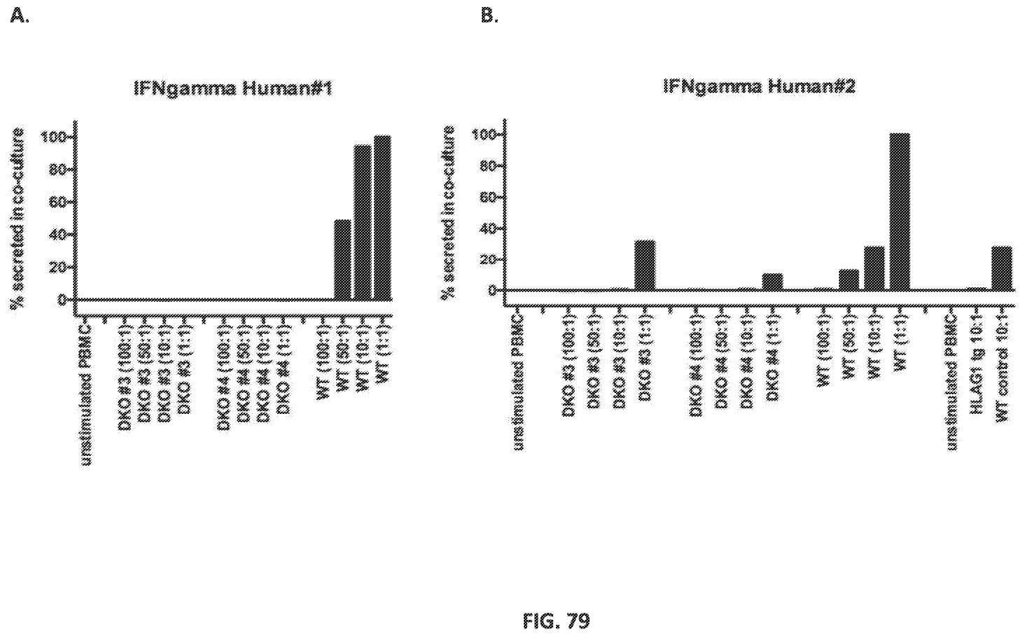

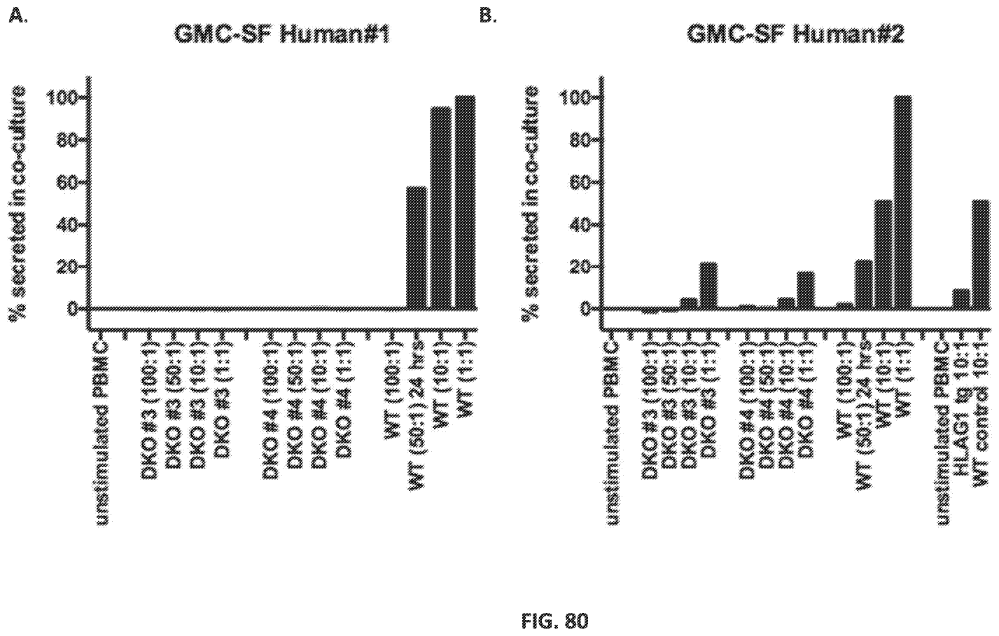

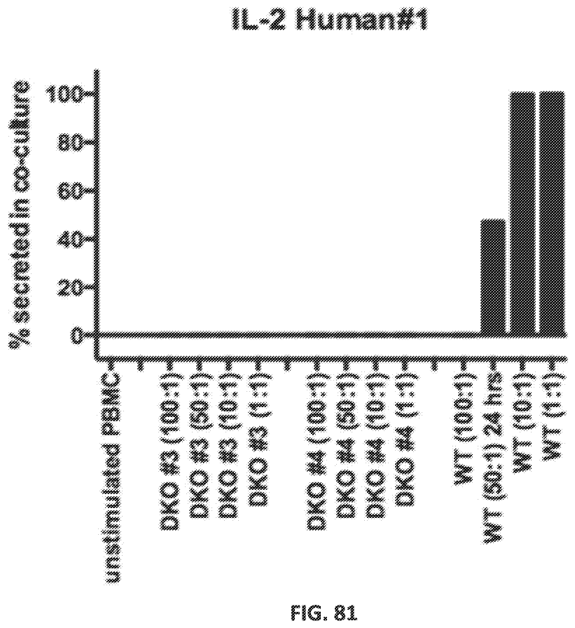

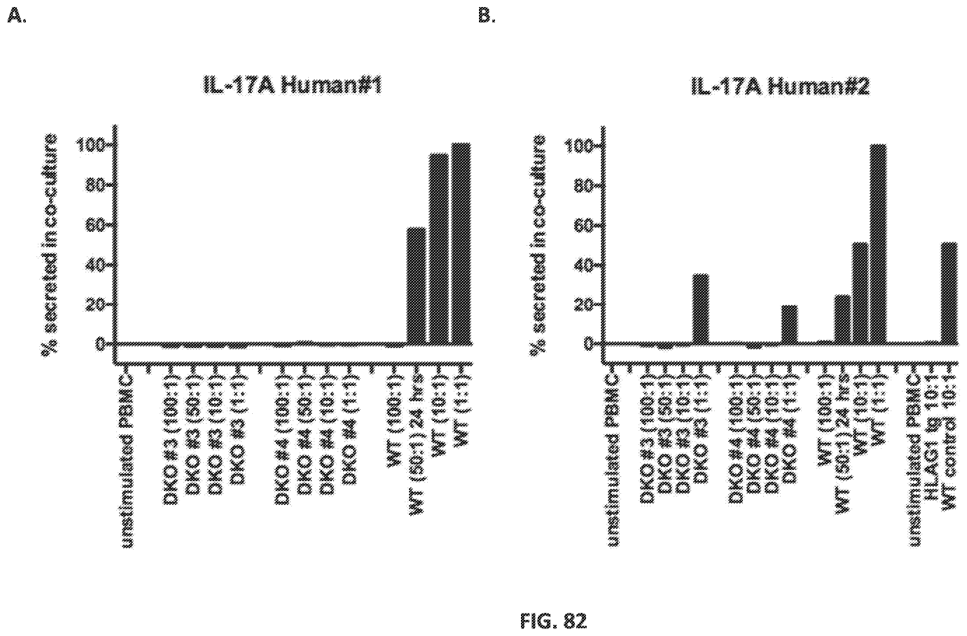

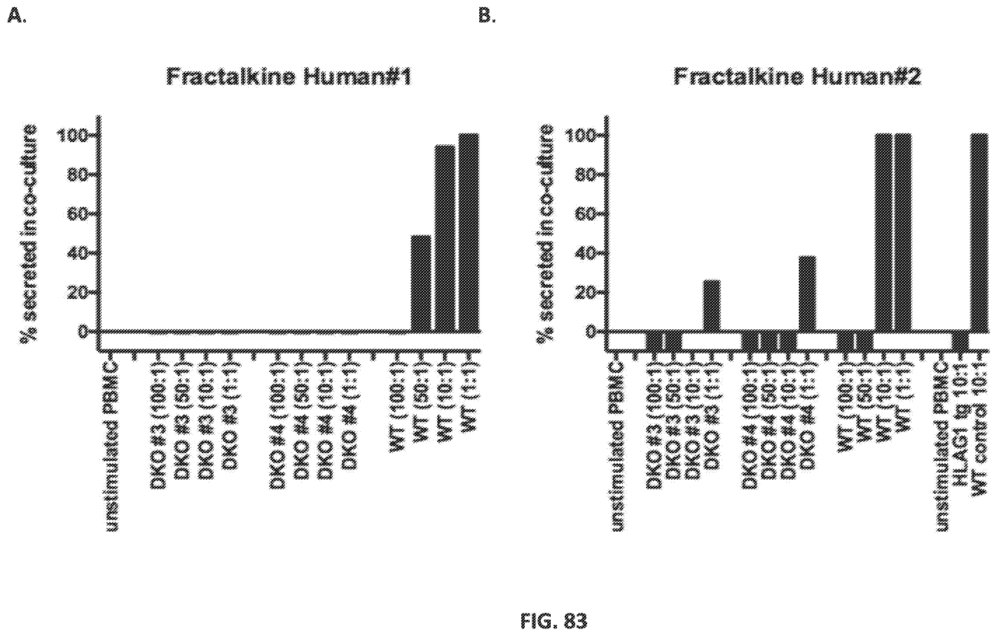

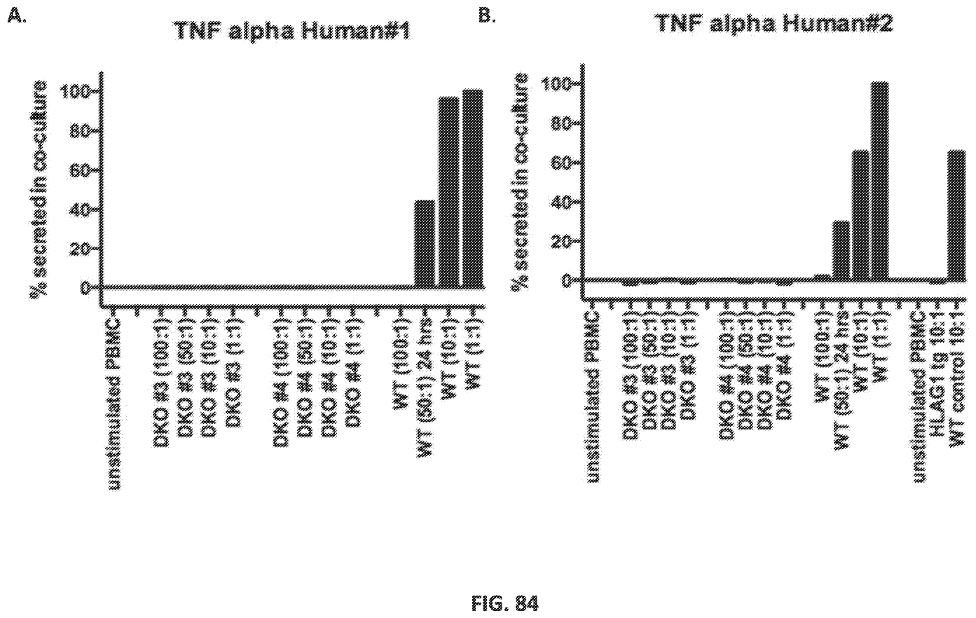

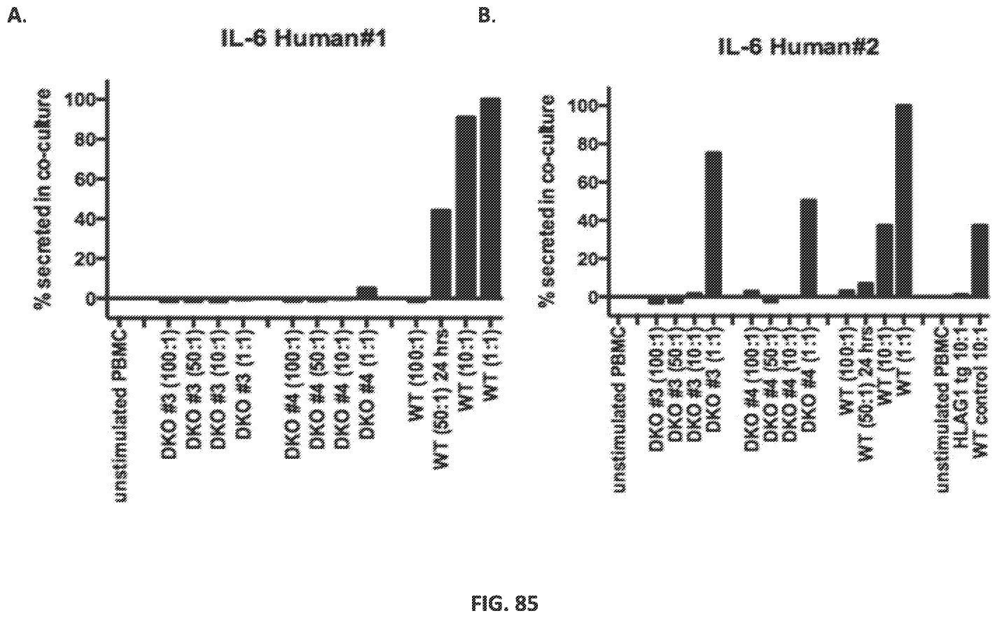

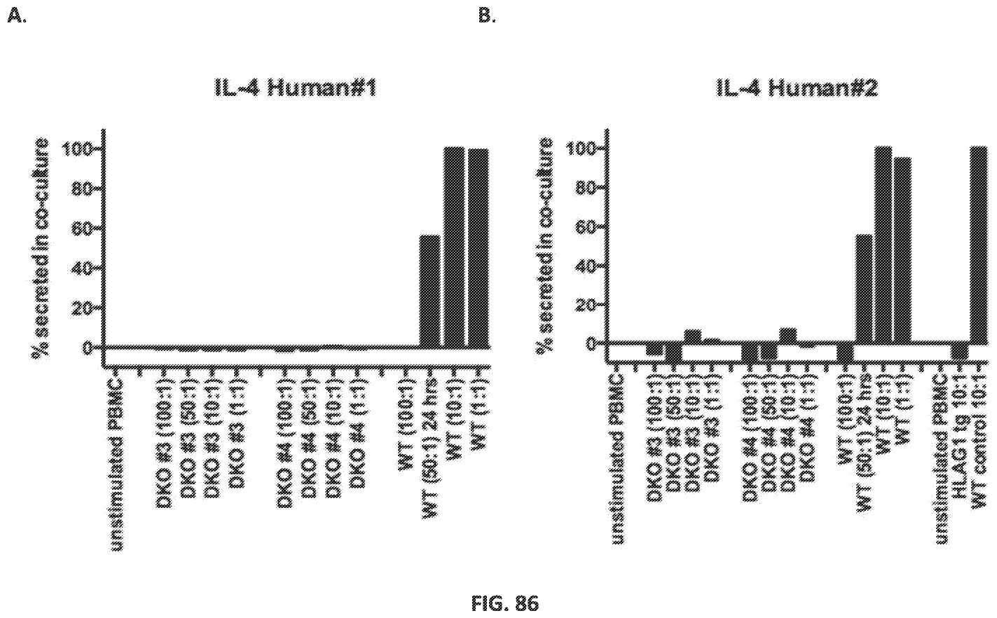

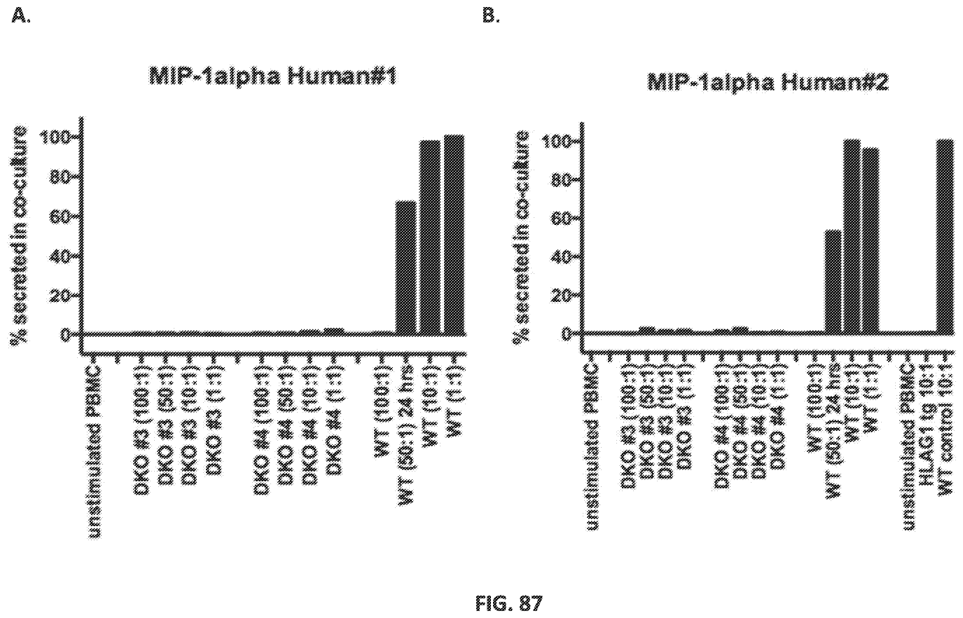

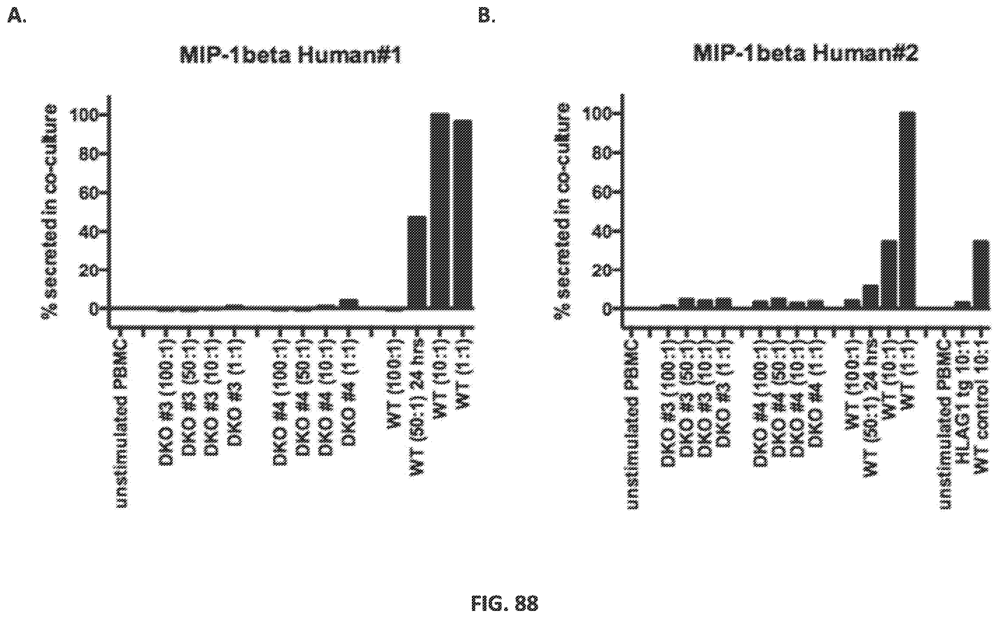

Mixed lymphocyte reaction co-cultures were carried out in flat-bottom, 96-well plates. Human CFSE labeled (2.50 μM/ml) PBMCs, were used as responders at 1-2×10⁵ cells/well/200 ul. Porcine fibroblast cells at 2000 to 1×10⁵ cells/well (with or without SLA-blocking with PT85 10 ug/ml) or with HLA-G transduced Porcine fibroblast/MLF cells were used as stimulators at stimulator-responder ratios of 100:1, 50:1, 10:1 and 1:10 (FIG. 53 and FIG. 54). MLR co-cultures were carried out for 24 hrs for cytokines (11-2, TNF-a and IFN-g) effector molecules (Perforin, Granzyme B LAMP-1/CD107a) expression and 5-6 days for measurement of T and B cells proliferation. In another parallel experiment total PBMCs cells were stimulated with and without PHA (2 ug/ml) as positive and unstimulated control respectively. Cultured cells were washed and stained with anti-CD3, anti-CD4 and anti-CD8 followed by formaldehyde fixation and washing and intracellular staining with anti perforin, granzyme B, IL-2, TNF-a and IFN-g (FIG. 66 to FIG. 74 and FIG. 79 to FIG. 86). BD FACS Canto II flow, were used to assessed the proliferative capacity of CD8 and CD4 T cells in response to SLA-I knockout porcine fibroblast (F3) compared to unmodified porcine fibroblast cells (FIG. 61 to FIG. 65 and FIG. 75 to FIG. 78). Data were analyzed using FACS diva/Flow Jo software (Tri star, San Diego, Calif., USA), and percentage CFSE dim/low was determined on pre gated CD8 T cells and CD4 T cells, FIG. 61 to FIG. 65. The gating strategy used to analyze data is shown in FIG. 60.

### Example 13: Testing HLA-G Transgene Expressing Pig Cells to Inhibit the Human T-Cell Proliferation Response

T cell proliferation was reduced following stimulation by porcine fibroblast treated with PT-85 blocking Abs compared to control unmodified porcine fibroblast/WT at ratios 10:1 of Human PBMCs and FC respectively. Substantial reduction in T cells (CD3/CD4/CD8) proliferation was observed when human responder were treated with SLA-I blocking PT-85 Abs or HLA-G expressing at 10:1 and 1:1 ratio. No much difference were seen in T cells proliferative response at 100:1 and 50:1 ratio compared to unmodified/WT porcine fibroblast. No substantial reduction in B cells proliferation either with blocking SLA-I with PT-85 or HLA-G expression.

### Example 14: Secreted Cytokine Profile after Mixed Lymphocyte Assay Measure by Luminex Human Cytokine Panel (HSTCMAG-28SK Human High Sensitivity T Cell)

To determine the cytokine profile of mixed lymphocytes to porcine genetically modified cells a co-culture assay was performed where the supernatant from day 24 mixed cell cultures and controls was collected and the luminex assay was performed. Following the manufacturers protocol, an aliquot of supernatant was removed and incubated with luminex bead for each cytokine, washed, and measured on a factory maintained luminex instrument. Double knock out (DKO) #3 and #4 are genetically and phenotypically GGTA1/NLRC5 knock out cells made separately. The HLAG1 transgenic cells were conducted in a separate experiment and therefore include matching unstimulated and wild type cell controls.

### Example 15: Genetic Modifications of GGTA1-10, Gal2-2 and NLRC5-6

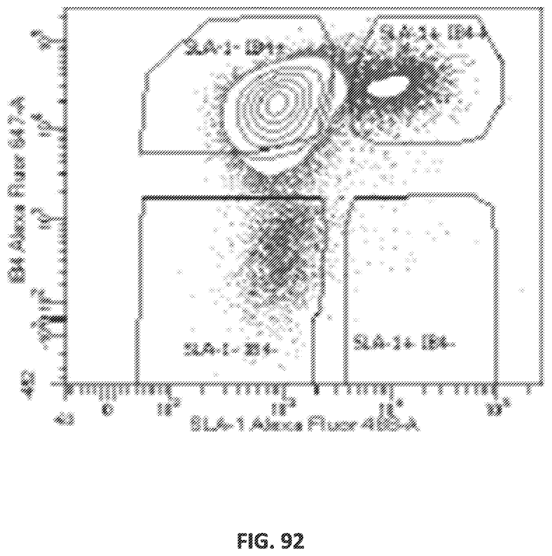

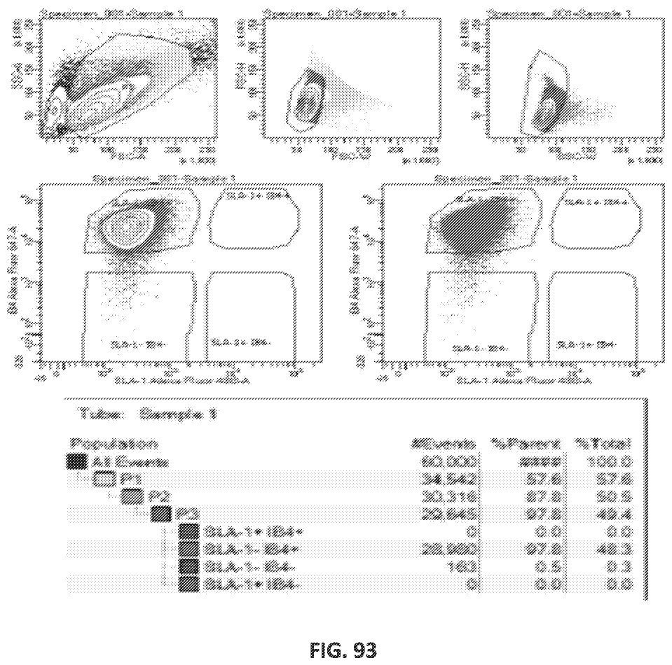

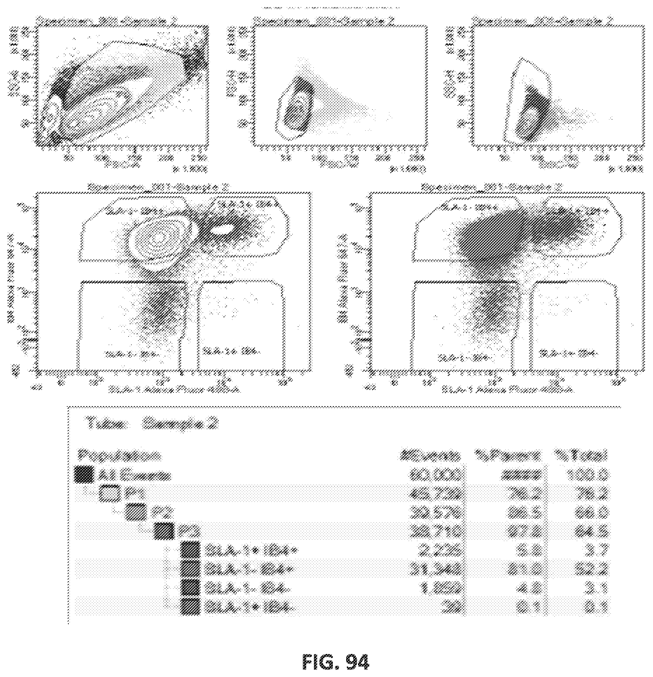

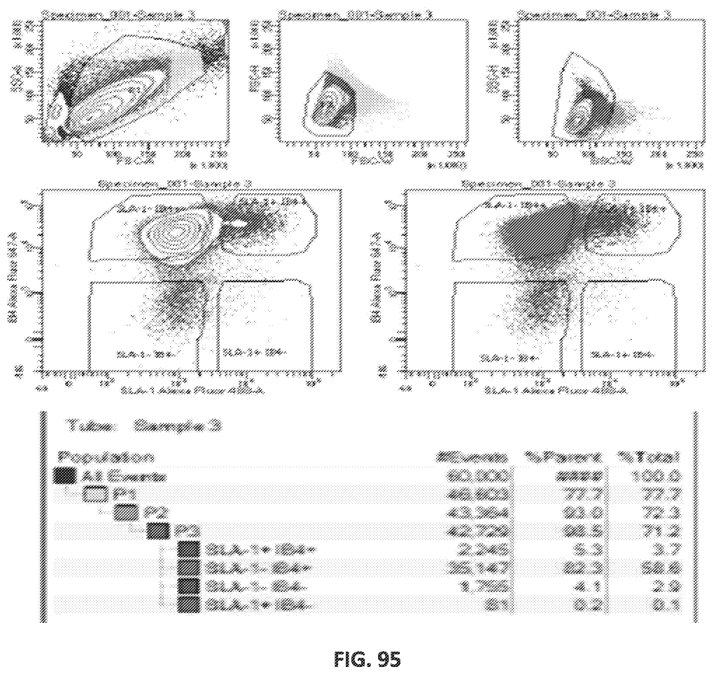

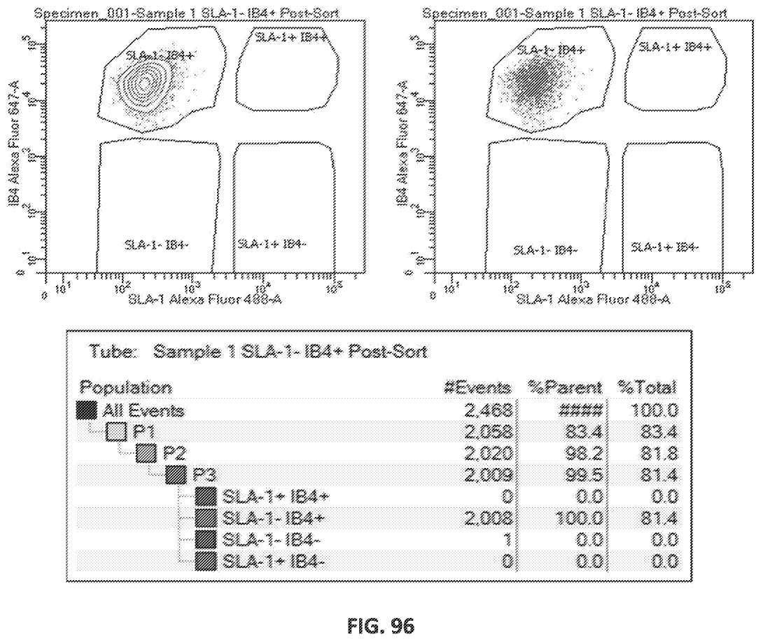

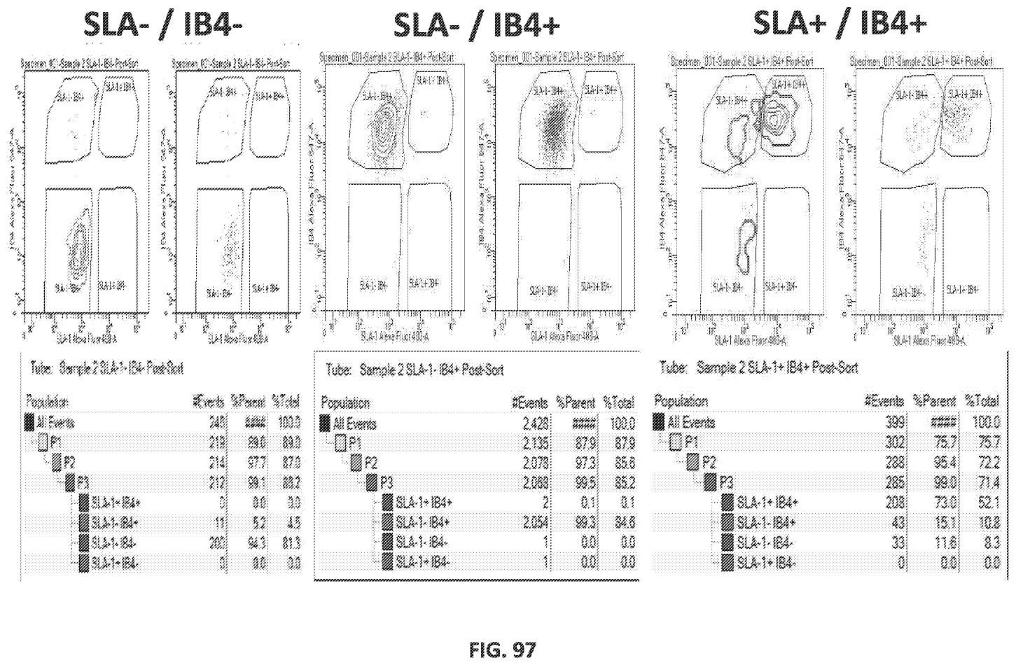

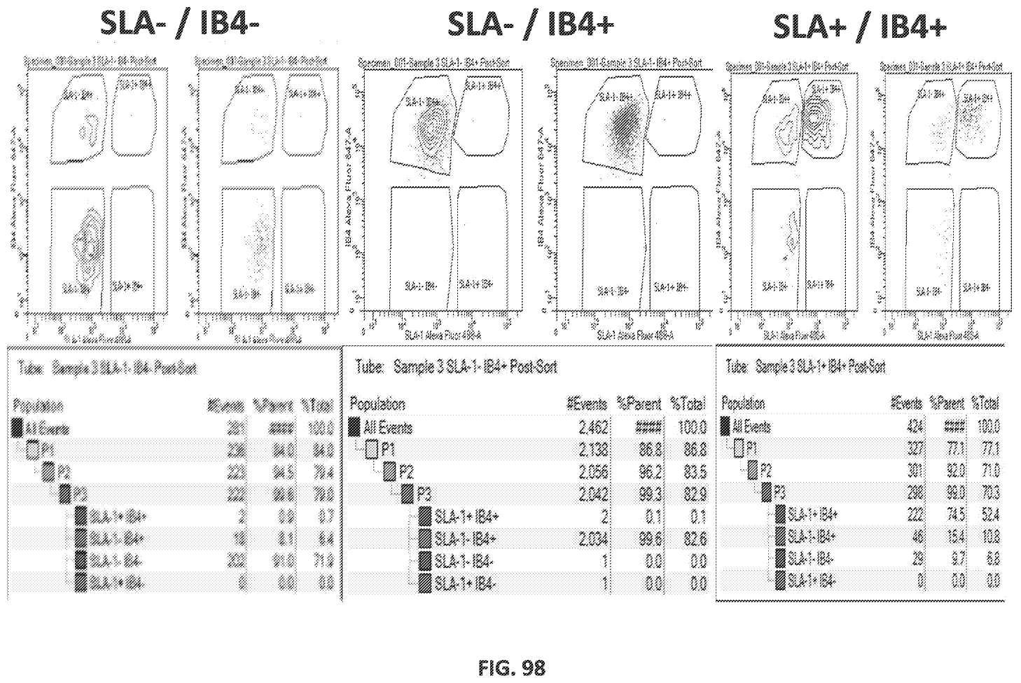

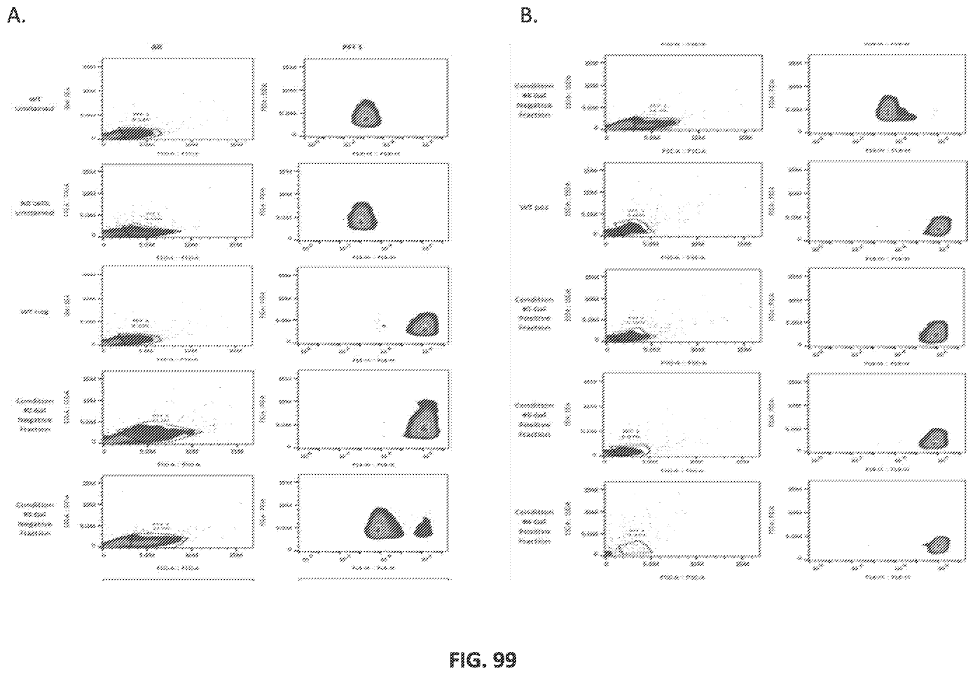

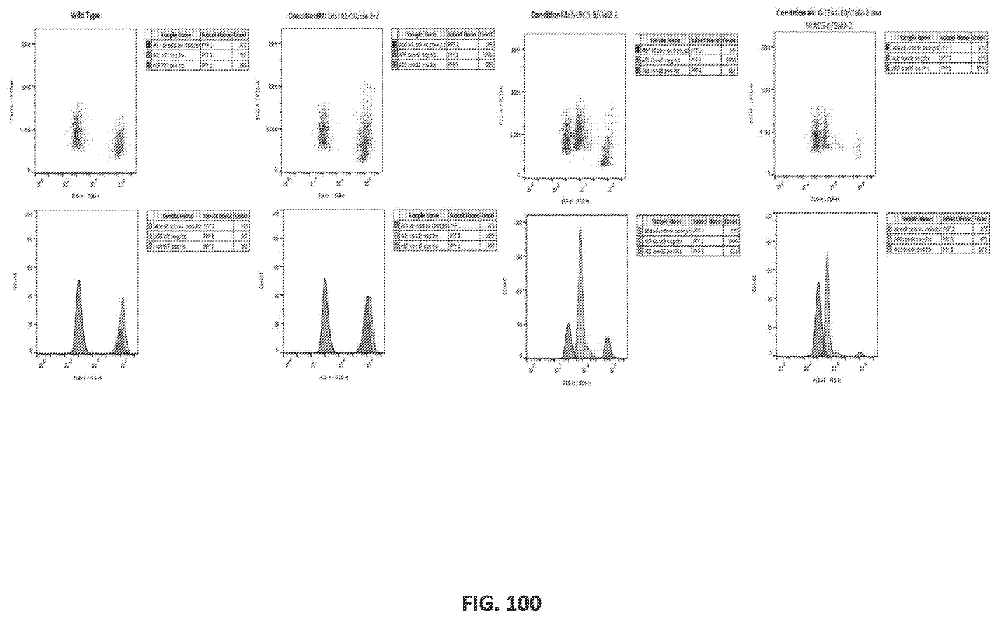

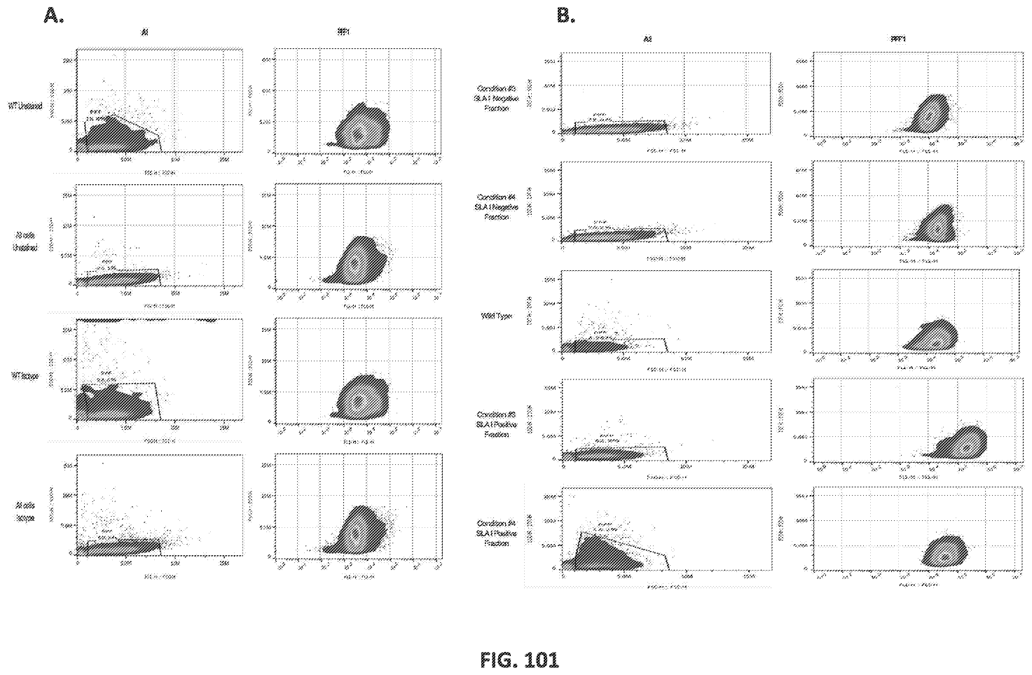

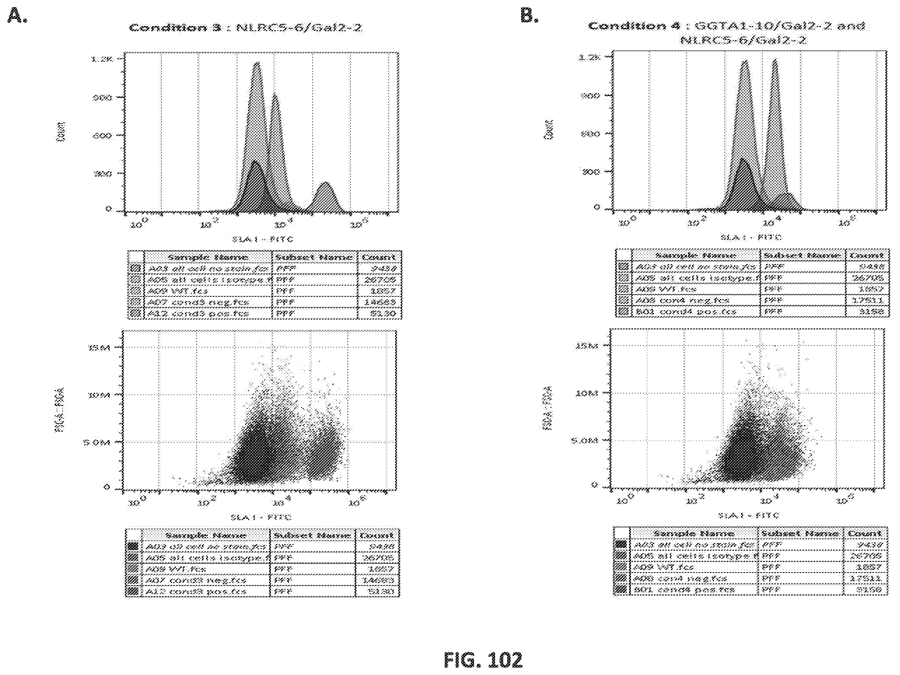

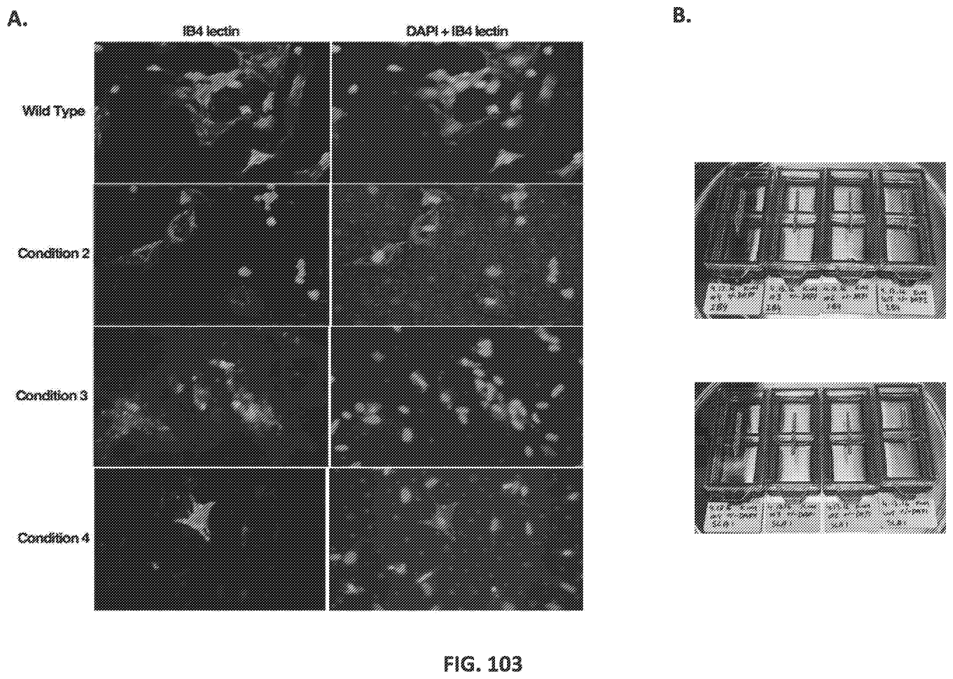

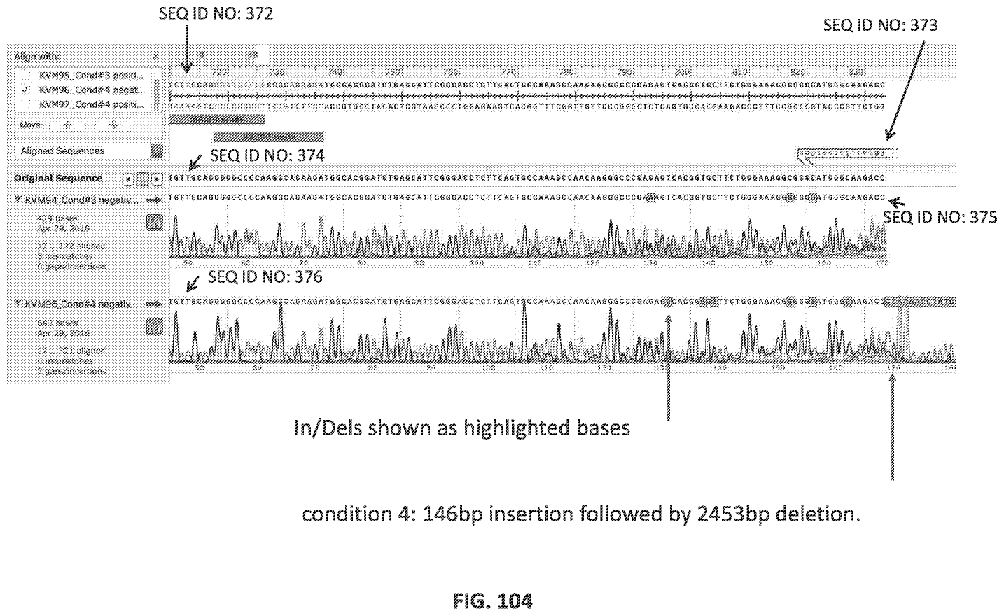

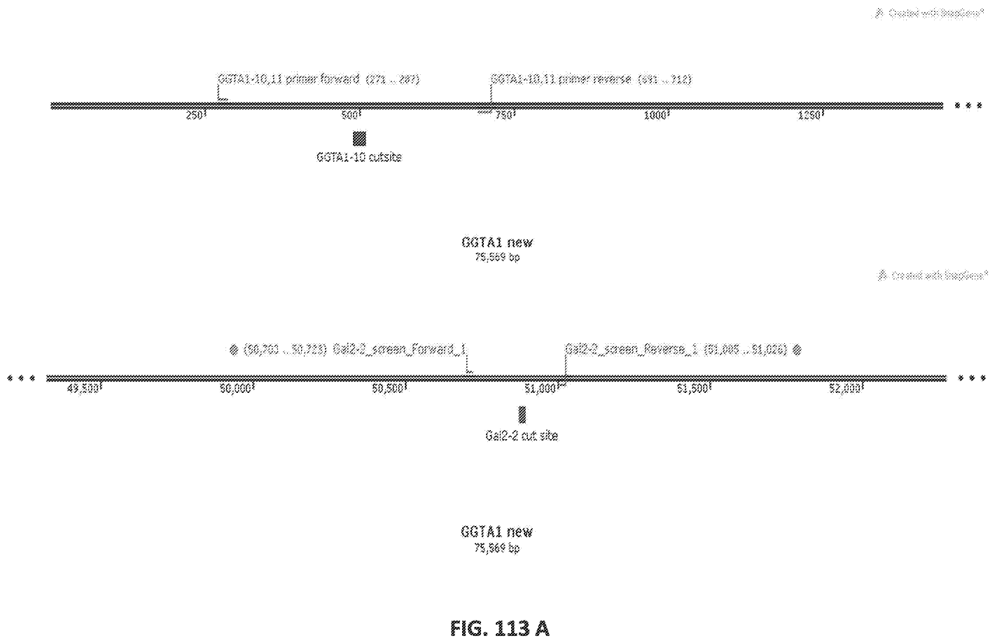

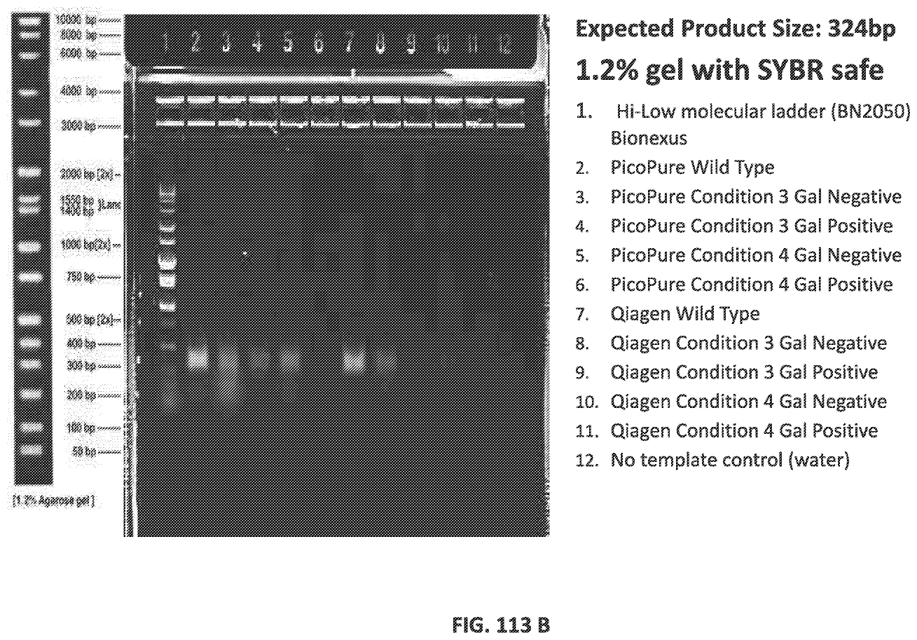

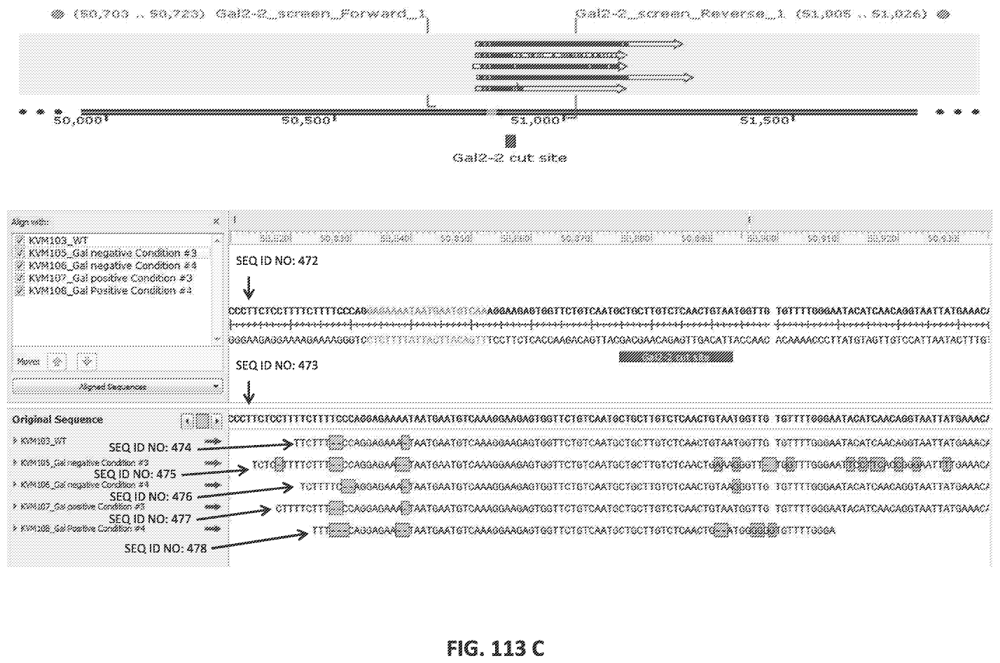

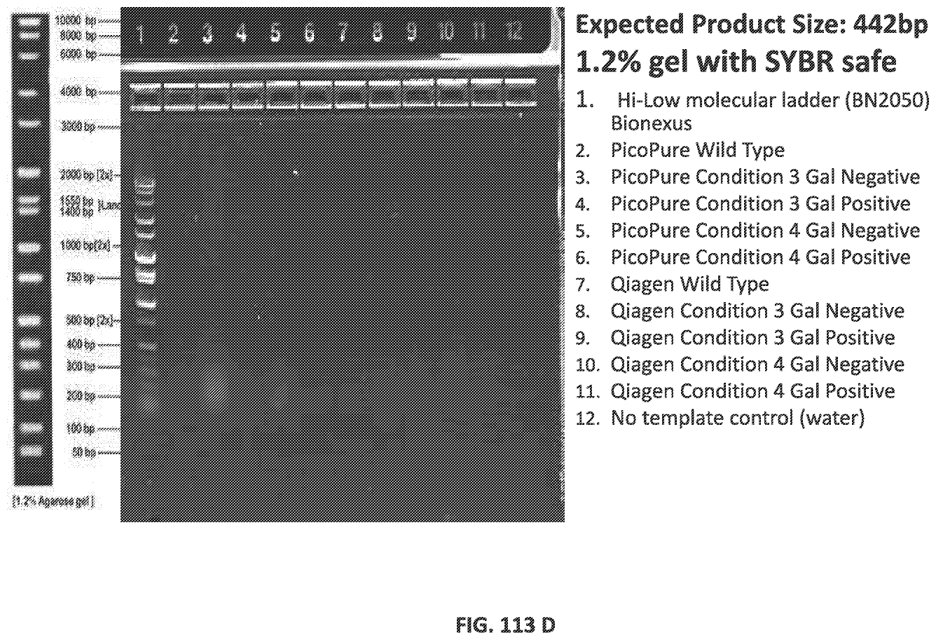

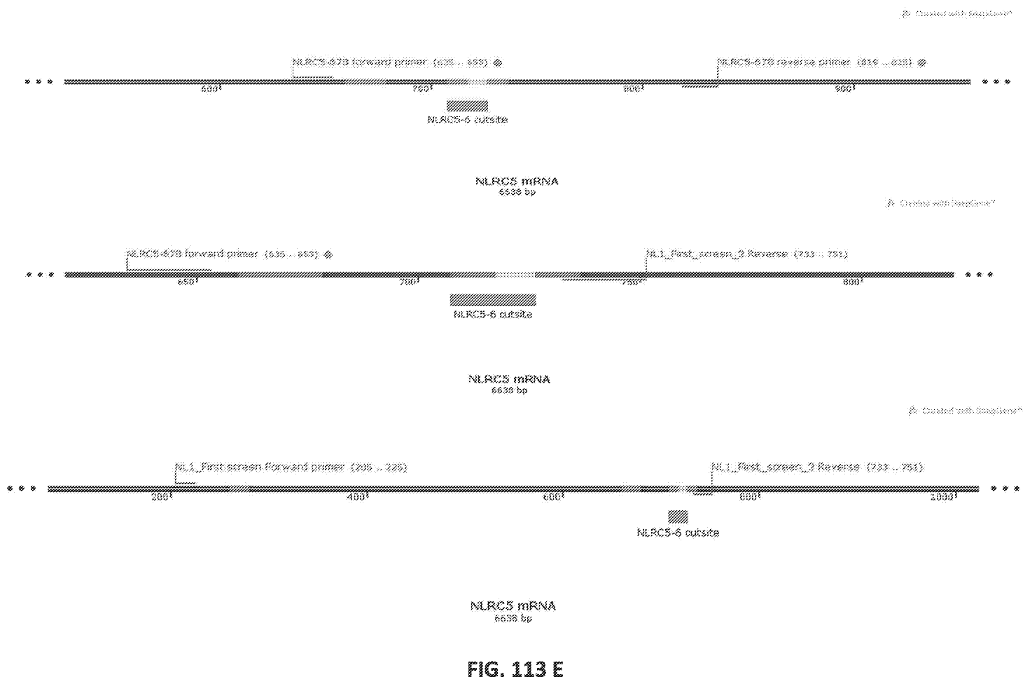

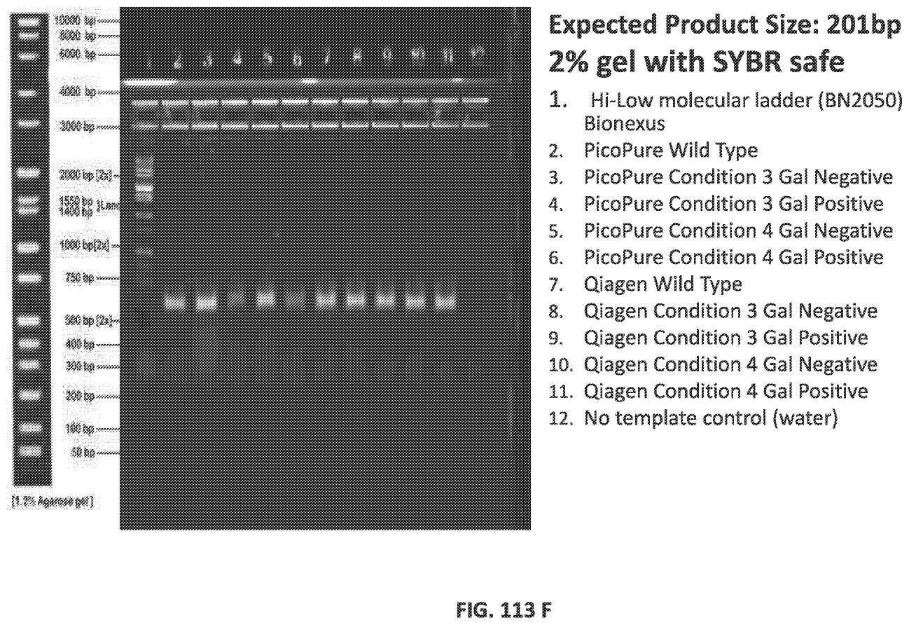

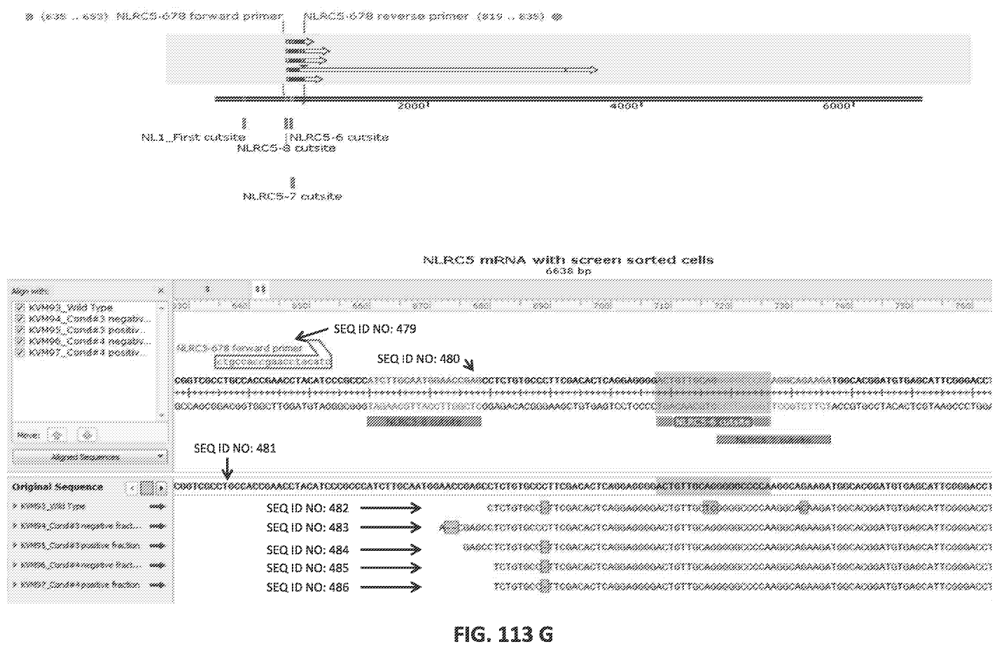

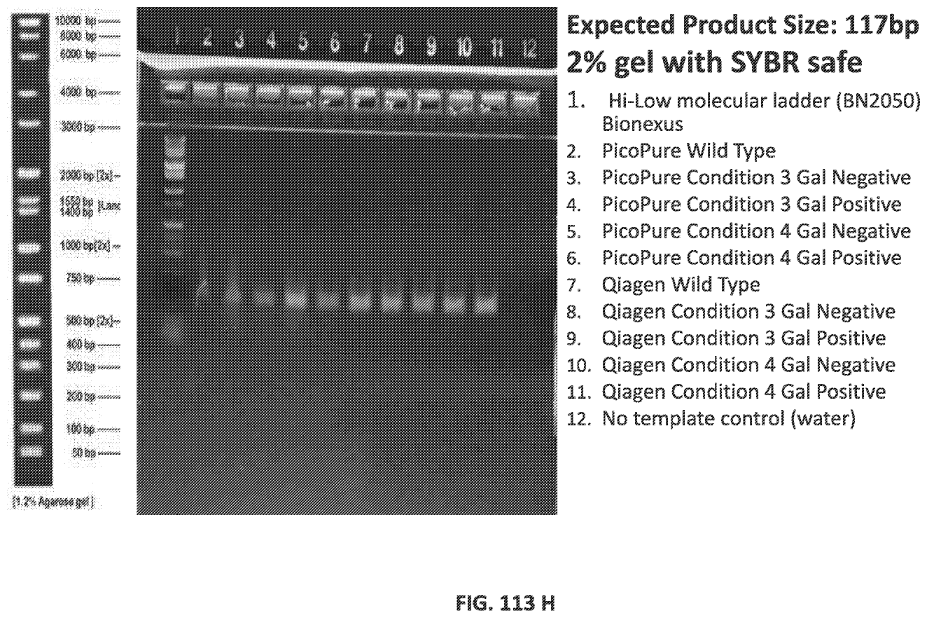

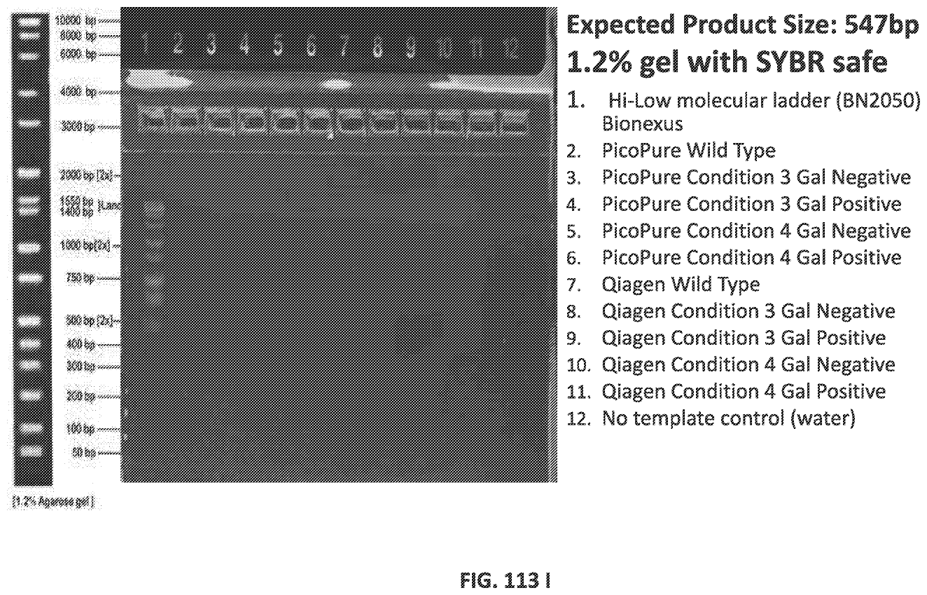

Primary porcine cells were transfected with: GGTA1-10/Gal2-2 (condition 2), NLRC5-6/Gal2-2 (condition 3), GGTA1-10/Gal2-2 and NLRC5-6/Gal2-2 (condition 4), or Condition 1: cells only (FIG. 90). Bead Selection of Negative cells by using magnetic bead sorting was performed using an IB4 lectin selective for terminal alpha-D-galactosyl residues (such as the product of GGTA1) (FIG. 91). The first bead selection was performed after 5 days followed by a second bead selection at day 8. Cell Sort Selection of Negative Cells using the sort machine from University of MN was done 7 days after transfection. Cells were stained with IB4 lectin Alexa Fluor 467 and SLA I FITC and analyzed by flow cytometry (FIG. 92 to FIG. 102). Confocal microscopy of the cultures is shown in FIG. 103 A. Additional data shows electrophoresis of sequencing confirmation is shown in FIG. 113A to 113 I.

TABLE 16
Exemplary sequencing primers for px333 plasmids

| SEQ | Forward sequence | SEQ | Reverse sequence |
|---|---|---|---|
| ID No. | (5′ to 3′) | ID No. | (5′ to 3′) |
| 241 | cttcgtgaaaccgctgtttatt | 242 | gactggaggactttgtcttctt |
| | gagcagagctcactagaacttg | | aagagacaagcctcagactaaac |
| 243 | ttccactctgggtgtatttaatct | 244 | ccggatccttaagccaaaga |
| 245 | gctcagcctagggtttcaat | 246 | atgagcaaggcaggaatgt |
| 247 | agtttgggactgcctcattt | 248 | ggagcagggaaacctgataaa |
| 249 | gccactgttccctcagc | 250 | cgttgcctatagcgtcttctt |
| | gacccgctctgcacaaa | | cagaggtaacgacgagaacaaa |
| 251 | gacccgctctgcacaaa | 252 | cagaggtaacgacgagaacaaa |
| 253 | ggagttacagggaatccgaatg | 254 | catgaagccaagatctaggaag |
| 255 | ctgctctgcaaacactcaga | 256 | tcagcagcagtacctcca |
| | tgagtgccaaggtgaagttct | | caaagcagtgcaggaagcag |
| | gagtgccaaggtgaagttct | | aagatggcacggatgtgag |
| 257 | ctgccaccgaacctacatc | 258 | gtggtcttgcccatgcc |
| 259 | catcagtcctggtgatgatcc | 260 | gatgagtgggaagatgacct |

TABLE 17
Exemplary Sequences of the first exon of NLRC5 and/or
B4GALNT2 gene to be targeted by guide RNA.

| SEQ | |
|---|---|
| ID No. | Genomic Sequence (5′ to 3′) |
| 261 | caggaggggactgttgcagggggccccaaggcagaagatggcacggatgtgagcattcgggacctcttcagtg |
| | ccaaagccaacaagggcccgagagtcacggtgcttctgggaaaggcgggcatgggcaagacc |
| 262 | atgctgcttgtctcaactgtaatggttgtgttttgggaatacatcaacaggtaattatgaaacatgatgaaatgatgttg |
| | atgaaagtctcctctaatctcctagttatcagccaagtcaccagcttgcattaaa |

### Example 16: Generation and Characterization of HLA-G Knockin Cells for Making Genetically Modified Animals of the Laurasiatheria Super Order

Cells of animals of the Laurasiatheria super order with HLA-G knocked in can be generated using CRISPR/Cas9-mediated gene editing technology. Knock in of HLA-G can include HLA-G1, HLA-G2, HLA-G3, HLA-G4, HLA-G5, HLA-G6, or HLA-G7. HLA-G can be inserted at a target locus. For example, HLA-G can be inserted into the Rosa26 locus of an animal of the Laurasiatheria super order. Alternatively, HLA-G can be inserted into another target locus such as a glycoprotein galactosyltransferase alpha 1,3 (GGTA1), a putative cytidine monophosphate-N-acetylneuraminic acid hydroxylase-like protein (CMAH), a β1,4 N-acetylgalactosaminyltransferase (B4GALNT2), a C—X—C motif chemokine 10 (CXCL10), a MHC class I polypeptide-related sequence A (MICA), a MHC class I polypeptide-related sequence B (MICB), a transporter associated with antigen processing 1 (TAP1), or a NOD-like receptor family CARD domain containing 5 (NLRC5). A knock-in of an HLA-G encoding sequence targeted to another gene can disrupt, or knock-out, that gene.

The target region for HLA-G insertion will be sequenced as described essentially in Example 2 above. Accurate sequence information will be used to design guide RNAs specific for the target region as described in Example 2. A plasmid, such as px330, expressing guide RNAs specific for the target region will be generated using methods described in Example 1 and Example 2. Alternatively, a plasmid, such as px333, simultaneously expressing two guide RNAs specific for the target region can be generated as described in Example 3.

As described in Example 8, the DNA sequence 1000 bp upstream (5′) and downstream (3′) from the target locus cut site will be confirmed. The left homologous arm will be designated as 1000 bp upstream of the cut site, and the right homologous arm will be designed as 1000 bp downstream of the cute site. Generation of homology directing fragments containing HLA-G and insertion of HLA-G at the target locus will be performed as described for HLA-G1 insertion at the Rosa26 locus in Example 8. The HLA-G sequence used can be transcribed as an mRNA with modifications in a 5′ and/or 3′ untranslated region. Such modifications can increase mRNA stability.

Cells of animals of the Laurasiatheria super order can have knock out of genes in combination with HLA-G knock-in. For example, GGTA1 and/or NLRC5 can be knocked out, and HLA-G can be knocked in. Thus, a GGTA1/NLRC5 knockout/HLA-G knock-in animal of the Laurasiatheria super order can be generated using methods similar to those described in Example 8. As described, the knock-in of an HLA-G encoding sequence can disrupt, or knock out, another gene (e.g., GGTA1 and/or NLRC5).

Animals of the Laurasiatheria super order can include an ungulate, such as an even-toed ungulate (e.g., pigs, peccaries, hippopotamuses, camels, llamas, chevrotains (mouse deer), deer, giraffes, pronghorn, antelopes, goat-antelopes (which include sheep, goats and others), or cattle) or an odd-toed ungulate (e.g., horse, tapirs, and rhinoceroses), a non-human primate (e.g., a monkey, or a chimpanzee), a Canidae (e.g., a dog) or a cat. Members of the Laurasiatheria superorder can include Eulipotyphla (hedgehogs, shrews, and moles), Perissodactyla (rhinoceroses, horses, and tapirs), Carnivora (carnivores, such as cats, dogs, and bears), Cetartiodactyla (artiodactyls and cetaceans), Chiroptera (bats), and Pholidota (pangolins).

TABLE 18
Sequences for SEQ ID NOs: 5-60

| | |
|---|---|
| SEQ ID NO: 5 | NLRC5 Genomic Sequence |

TGGAAACAACATGAACACTGTGAGCTCCCGGGAGTTCAGTCAGATCCACTGAGGTAGTGGCCGGGTCCAGCGGCCTT
GCCTAACTTGGCAGTCCCCACCCGCTGCATCCTTAGATCTGGCTTTGTCCCTTACACAGGACAGCCCAGGCCTGTGA
TCCCCAAGGTCAGGCTAACGCTACCTGGACCTGGGCTCTAAGACCTGGGAAGCTACAGGAGGGGTGAGCCAGTTCCC
AGATTGGGAAAACTGAGGCTTGAGGCGAGAGGATAGTCATCCACAAGCCTCGTGGCTAAATCCCTGGCTTGGCCCAG
GGCCCTGGACCTCAGGCCACTGGGCTGATCAGTGCTTGTATGCTTTCCTCATCGCACTTGTTTGGAAGACATTCCCT
GGTTTAGCTGCTCTGGGATGGTAATCTATAAATACATACTTTGTTTAAAAAATTAATAAATTAAATCTTGGACCAGC
ATGAGGGCATCTGGCCAGCCACATGGCATATGACATGGACATTTGCCACGTCTCAAATATGGACTGCCCATCACATG
TAGTGCTAGGACCCATGCCAACAACCCACAGGCCACACTGCAGGTTTCATGCAATGTCACATGGAACGCTGCCACGN
NNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNTCACGCCACGACATCCTCACTGTGCTGCATATTCCCGACTGGTCATGCATGTCAT
GTGTGATGGAGGGTGGTCTGTTGGCCATANNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNTCTGAAGACCGTGCCTGGAAAACGG
CGTCTCTCCCTCCCGGAACAGTGTGCCGGGACAGCCAGCTGAGGCTCTTTTCCTGAGCCCTCTATCCTGGGGGATGG
AAGCGGACATCACTTGGCTGTATTGGAAGGGTCTTGCGGGGGCCGTCAAGCATCCCAGGGGACCTGTGGCTGATGGT
CGAAGAAAGCAAAGTCCAGCCTGGGCTCCCGGCTCTGCAGATGCTGGGCCGTGTCCTGGGGGATGGGGTTATTCCAC
AGGCTGCGGGGCACAGAGACAGACATTCAGCACTGGGAGCTGTTCACTTGTCCTTGTCTCTACCCTCTGTCCAACCC
ACAGATGGGGAAACTGAGGCCCCAAAGGGGAAGAGCTGTTCCCAGAGTTACCTGGCAGGTAGGAGCAGGTGTTAGAC
CAGCATGGCTACCTTAGGGAGATGGTATCCCCCATGCCCACCCCAACTTCTTCCACTCACTCTTCTTCCCTGGAAGC
TAGTGATGCCAGCTGGGCCATGCTCATATGACACATTGTGCAAATAAGGAGAAAGCCCCCCCCTTTATTTCTTTTTG
TTTTTTTTTTTTTTACCATTTCTTGGGCCGCTCCCGCGGCATATGGAGATTCCCAGGCTAGGGGTCGAATCGGAGCT
GTAGCCGCCAGCCTACGCCAGAGCCACAGCAACTCGGGATCCGAGCTGCATCTGCGACCTGCACCACAGCTCATGGC
AACGCCGGATCGTTAACCCACTGAGCAGGGCCAGGGATCGAACCCGCAACCTCATGGTTCCTAGTTGGATTCATTAA
CCACTGTGCCACGATGGGAACTCTGAAAGCTCCCCCTTTTTAGACACTTTATTTCTATCTTCTGAAACTGTCATACT
GAGTTTTATAGAGCGAGACCNCCCCCTTTTTAAGACACTTTATTTCTATCTTCTGAAACTGTCGTAATATACTGAGT
TTTATAGAGCGAGACCCTTCACTACTACCAGAAACCTAACACGTCAACGGTGTGAACAGTGTCCTTTAGATGCAAGG
CCTTGGTACAGTGTGCAGCCTGTGCAACTGTACGTGGTGGCTGTGATTACAGTTATCATTTTAAGCACTTGCTATGT
GCCAGGCATTGTACTCAGTGCTTTGTAGAATCATTTAGTCTGCAGAGCGCCCATCTAAGGCTGATATGATCATTGTC
TCCAGTTTACAAATGAGGAAACCGAGGTTCAGGGAGGTTGAGTTACTGAGGCAAAGTTACACAGTCAGCAACCAGTA
GAGCTGGGATTTGATCCAGGTCTGCTGGCTGCCACATTCCTGGTGGAGTGGGCCAAATCTCCTTTGATAATCCCCAA
TCCAGGAGTTCCTGTTGTGGCGCAGCAGAAATGAATCCGACTAGTAACCATAAGGTTGCAGGTTCAATCCCTGGTCT
TGCTCAGTGGGTTAAGGATCTGGCGTTGCTATGAGCTGTGGTGTAGGTTGAAGATGCACCTCAGATCCCACAATGCT
GTGGCTATGGCGTAGGCTGGCGGATGTAGCTCTGATTGGACCCCTAGCCTGGGAATCTCCATATGCTGCAGGTGCGG
CCCTAAAAAAGCAATAAATAAGTAAATAGATAACCCTCAACCCAGGTCCTGCCTCCTCCTACAGAAAGTTCCTTTGC
ATTGTAGAGGCTGCTGTGGCCCCCACCTCCCACCATCCTCGCCCCTGCAAGTCCTGTTACCGAATGACTTGGATGCC
AGAGCCCTGAGCCAGCCCTTCAGCCAGGAGCCAGGCTCCATGAG

| | |
|---|---|
| SEQ ID NO: 6 | NLRC5 cDNA Sequence |

GGGCCTGTCCTATGGAAAGAACCTGCAAGTCCAGCACAGGGGCTTGGCCGGGAACCCATGAGACCCCCTCTGGGGAC
ATCCTAGGACATCTGTGATGAATCAGGAAGCAGGGCTGGCTCCTCATGGACCCCATTAGTCGCCACCTGGGCACCAA
GAACCTGTGGGGATGGCTCGTGAGGCTGCTCTGCAAACACTCAGAATGGCTGAGTGCCAAGGTGAAGTTCTTCCTCC
CCAACATGGACCTGGGTGCCAGGAACGAGGCCTCAGACCCCACACAGAGGGTCGTCCTACAACTCAGAAAACTGCGT
ACCCAGAGTCAGATCACCTGGCAGGCGTTCATCCACTGTGTGTGCATGGAGCTGGACGTGCCGCTGGACCTGGAGGT
ACTGCTGCTGAGCACCTGGGGCCACGGAGAAGGGCTCCCCAGTCAGCTGGAAGCTGATGAGGAGCACCCACCTGAGT
CTCAGCCCCACTCTGGCCTCAAGCGGCCACATCAGAGCTGTGGGCCCTCCCCTCGCCCAAAGCAGTGCAGGAAGCAG
CAGCGAGAACTGGCCAAGAGGTACCTGCAGCTGCTGAGAACGTTTGCCCAGCAGCGTTACGACAGCAGGAGCCCTGG
GCCAGGACAGCCGGTCGCCTGCCACCGAACCTACATCCCGCCCATCTTGCAATGGAACCGAGCCTCTGTGCCCTTCG
ACACTCAGGAGGGGACTGTTGCAGGGGGCCCCAAGGCAGAAGATGGCACGGATGTGAGCATTCGGGACCTCTTCAGT
GCCAAAGCCAACAAGGGCCCGAGAGTCACGGTGCTTCTGGGAAAGGCGGGCATGGGCAAGACCACGCTGGCCCACCG
GCTCTGCCAAGAGTGGGCCGATGGTCAGCTGGAGCGCTTCCAGGCCCTGTTCCTTTTCGAATTCCGCCAGCTCAACC
TGATCACAAACTTCCTGATGCTGCCACAGCTCCTTTTTGATCTGTACCTGAGGCCCGAGGCGGGCCCAGAGGCAGTC
TTCCAGTACCTGGAGGAGAATGCTAATAAAATCCTGCTCATCTTTGATGGGCTGGACGAGGTCCTCCACCCCGGCTC
CAGCAAGGAGGCTGCAGATCCTGAGGCCTCGGCGTCAGCCCTCACCCTCTTCTCCCGCCTCTGCCATGGGACCCTCC
TGCCCGGCTGCTGGGTCATGACCACCTCCCGTCCAGGGAAGCTGCCCGCCTGCCTGCCCACAGAGGTGGTCACGGTC
AGCATGTGGGGCTTTGACGGACCACGGGTGGAGGAGTACGTGAGCCGCTTCTTCAGCGACCAGCCAGTCCAGGAGGC
GGCCCTCGCGGAGCTGCGGGCCAGCTGGCATCTCTGGAGCATGTGTGTGGTGCCCGCGCTGTGCCAGGTCGCCTGCC
TCTGCCTCCACCATCTGCTCCCAGGCCGCTCTCCAGGCCAGTCTGCAGCCCTCCTGCCCACCGTGACCCAGAGCTAC
GTGCAGATGGTGCTTTCCCTCAGCCCCCAAGGGTTCCTGCCTGCCGAGTCCCTGATGGGCCTCGGGGAGGTGGCCCT
GTGGGGCCTGGAGACGGGGAAGGTTGTCTTCACTGCAGGAGACATCCCTCCACCCACGATGGCCTTCGCGGCGGCCC
TCGGCCTGCTCACCTCCTTCTGTGTGTACACGGAACCCGGGCACCAGGAGACAGGCTACGTCTTCACCCACCTCAGC
CTGCAGCAGTTTTTGGCTGCCCTGCACCTGATGGCCAGCCCCAAGGTGGACAGAGACACACTTGCCCAACATGTCAC
CCTCAATTCTCGCTGGGTGCTGCGGACCAAAGCTAGGCTGGGCCTCTTAGACCACCACCTTCCCACCTTTCTGGCCG
GCCTGGCCTCCTGCGCCTGCCACCCCTTCCTCACACCCCTGGCACAGCAGGAGGAGGTGTGGGTGCGTGCCAGGCAG
GCGGCAGTCATGCAAGCCTTGGAGAAGTTGGCCACTCGCAAGCTGACGGGGCCAAAGCTGATAGAGCTATGTCACTG
CGTGGCTGAGACACAGAAGCCGGAGCTGGCCAGCCTCGTGGCCCAGAGCCTCCCCCATCACCTCTCCTTCCGCAACT
TTCTGCTGACCTATGCCGACCTGGCTGCCCTGACCAACATCCTCGGGCACAGGGATGCCCCCATCCACCTGGATTTT
GAGGGCTGCCCCTTGGAGCCACACTGTCCTGAAGCCCTGGCAGGCTGCGAGCAGGTGGAGAATCTCAGCTTTAAGAG
CAGGAAGTGTGGGGATGCCTTTGCTGAAGCCCTCTCCAGGAGTTTGCCAACAATGGGGAGCCTGAAGAAGCTGGGGT
TGTCAGGAAGTAGGATCACTGCCCGAGGCATCAGCCACCTGGTGCGGGCTTTGCCCCTCTGTCCACAGCTGGAAGAG
GTCAGCTTTCAGGACAACCAGCTCAAGGACGGGGAGGTCCTGAACATCGTGGAAATACTTCCCCACCTGCCGCAGCT
CCGGATGCTTGACCTGAGCCGCAACAGTGTCTCCGTGTCAACTCTCCTCTCCTTGACAAAGGTGGCAGTCACGTACC
CTACCATTAGGAAGCTGCAGGTCAGGGAGACAGACCTCGTCTTCCTTCTCTCCCCACCTACAGAGATGACCACAGAG
CTACAAAGAGACCCAGACCTACAGGAAAATGCCAGCCAGAGGAAAGAGGCTCAGAGGAGAAGCCTGGAGCTCAGGCT
CCAGAAGTGTCAGCTCAGTGTCTATGATGTGAAGCTGCTCCTCGCCCAGCTCCGGATGGGTCCACAGCTGGATGAAG
TGGACCTCTCAGGGAACCAGCTGGAAGATGAAGGCTGTCAACTGGTGGCAGAGGCTGCGCCCCAGCTGCACATTGCC
AGGAAGCTGGACCTCAGCGACAATGGGCTTTCTGTGGCTGGGATGCAACGTGTGCTGAGTGCAGTGAGAACCTGCCG
GACCCTGGCAGAGCTACACATCAGTCTGCTGCACAAAACCGTGGTGCTCATGTTTGCCCCAGAACCAGAGGAGCAGG
AGGGGATCCAGAAGAGGCTGACACATTGTGGCCTGCAAGCCCAGCACCTTGAGCAGCTCTGCAAAGCGCTGGGAGGA
AGTTGCCACCTCAAGTACCTCGATTTATCAGGCAATGCTCTGGGGGACGAAGGTGTGGCCCTGCTGGCTCAGCTGCT
CCCCGGGCTTGGTGCCCTGCAGCTGCTGAACCTCAGTGAGAACGGTTTGTCCCTGGATGCTGTGTTCAGTTTGACCC
AGTGCTTCTCTACAGTGCGGTGGCTTCAGCGCTTGGACTTCAGCTCTGAGAGCCAGCACGTCATCCTGAGCGGTGAC
AGCAGAGGCAGGCATCTCTTGGCTGGCGGATCTTTGCCAGAGTTTCAAGCTGGAGCCCAGTTCTTGGGGTTCCGTCA
GCGCCGCATCCCCAGGAGCTTCTGCCTCAAGGAGTGTCAGCTGGAGCCCCCGAGCCTCTCCCGCCTCTGTGAGACTC
TGGAGAAGTGCCCGGGGCCTCTGGAAGTCGAATTGTTCTGCAAGGTCCTGAGTGACCAGAGCCTGGAGACCCTGCTG
CATCACCTTCCCCGGCTCCCCCAACTAAGCCTGCTGCAGCTGAGCCAGACGGGACTGTCCCAAAGGAGCCCCCTCCT
GCTGGCCGACCTCTTCAGCCTGTACCCACGGGTTCAGAAGGTGGATCTCAGGTCCCTCCATCACATGACTCTGCACT
TCAGGTTTAGCGAGGAGCAGGAAGGCGGATGCTGTGGCAGGTTCACAGGCTGTGGCCTCAGCCAGGAGCACATGGAG
CCGCTGTGTTGGTCGCTGAGCAAGTGTGAGGACCTCAGCCAACTGGACCTCTCCGCCAACCTGCTGGGTGATGACGG
GCTCAGGTCCCTCCTGGAATGTCTCCCTCAGGTGCCCATCTCCGGTTCGCTTGATCTGAGTCACAACGGCATCTCTC
AGGAAAGTGCCCTCCGCCTGGTGGAAACCCTTCCCTCCTGCCCACGTGTCCGGGAGGCCTCGGTGAACCCGGGCTCC
AAGCAGACCTTCTGGATTCACTTCTCCCGAAAGGAGGAGGCTAGGAAGACACTAAGGCTGAGTGAGTGCAGCTTCAG
GCCAGAGCACGTGCCCAGACTGGCCACCGGCCTGAGCCAGGCCCTGCAGCTGACAGAGCTCACGTTGAACCAGGGCT
GCCTGGGCCTGGAGCAGCTGACTATCCTCCTGGGCCTGCTGAAGTGGCCGGCGGGGCTGCTGACTCTCAGGGTAGAG
GAGCCGTGGGTGGGCAGAGCCGGAGTGCTCACCCTGCTGGAAGTCCGTGCCCACGCCTCAGGCAACGTCACTGAAAT
AAGCATCTCTGAGACCCAGGAGCAGCTCTGTATGCAGCTGGAATTTCCCCATCAGGAGAACCCAGAAGCCGTGGCCC
TCAGGTTGGCTCATTGTGATCTCGGGACCCACCACAGCCTCCTTGTCAGGGAGCTAATGGAGACATGCGCCAGGCTG
CGGCAGCTCAGCTTGTCCCAGGTGAAGCTCTGCAAGGCCAGCTCTCTGCTGCTGCAAAGCCTCCTGCTGTCCCTCTC
TGAGCTGAAGAACTTCCGGCTGACCTCCAGCTGTGTGAGCTCTGATGGGCTAGCCCACCTGACATTTGGTCTGAGCC
ATTGTCACCACCTGGAGGAGCTGGACTTGTCTAACAATCAATTTGGCAAGGAGGACACCAAGGTGCTGATGGGAGCC
CTTGAGGGCAAATGCTGGCTGAAGAGGCTTGACCTCAGCCACTTGCCTCTGAGCAGCTCCACCCTGGCCGCGCTCAT
TCAAGGACTGAGCCACATGAGCCTCCTGCAGAGCCTCCGTCTAAGCAGGAGCGGCGTTGATGACATCGGCTGCTGCC
ACCTCTCCGAGGCGCTCAGAGCTGCCACCAGCTTGGTGGAGCTGGGCTTGAGCCACAACCAGATCGGAGACGCCGGT
GCCCAGCACTTAGCTGCCATCCTGCCAGGGCTGCCTGAGCTCAGGAAGATAGACCTCTCAGCCAATGGCATCGGCCC
GGCAGGGGGAGTGCGGTTGGCGGAGTCCCTCACCCTTTGCGAGCACCTGGAGGAGCTGATGCTTGACTACAATGCTC
TGGGAGATCTCACAGCCCTGGGGCTGGCCCGAGGGTTGCCTCAGCACCTGAGGGTCCTGCACCTGCGGTCCAGCCAC
CTGGGCCCAGAGGGGGCGCTGAGCCTGGGCCAGGCACTGGATGGATGCCCATACGTGGAAGAGATCAACTTGGCCGA
GAACAGCCTGGCTGGAGGGATCCCACATTTCTGTCAGGGGCTCCCGATGCTCCGGCAGATAGACCTGATGTCATGTG
AGATTGACAACCAGACTGCCAAGCCCCTCGCCGCCAGCTTCGTGCTCTGCCCAGCCCTGGAAGAAATCATGCTGTCC
TGGAATCTGCTCGGTGACGAGGCAGCTGCTGAGCTGGCCCAGGTCCTGCCGCGGATGGGCCGACTGAAGAGAGTGGA
CCTGGAGAAGAATCGGATCACAGCTCACGGAGCCTGGCTCCTGGCTGAAGGGCTGGCTCAGGGCTCTGGCATCCAAG
TCATTCGCCTGTGGAATAACCCCATCCCCCAGGACACGGCCCAGCATCTGCAGAGCCGGGAGCCCAGGCTGGACTTT
GCTTTCTTCGACCATCAGCCACAGGTCCCCTGGGATGCTTGACGGCCCCCGCAAGACCCTTCCAATACAGCCAAGTG
ATGTCCGCTTCCATCCCCCAGGATAGAGGGCTCAGGAAAAGAGCCTCAGCTGGCTGTCCCGGCACACTGTTCCGGGA
GGGAGAGACGCCGTTTTCCAGGCACGGTCTTCAGAATGGACTTTATGGGCGACAAAGAGCCTACCATGGCCAACAGA
CCACCCTCCATCACACATGACATGCATGACCAGTCGGGAATATGCAGCACAGTGAGGATGTCGTGGCGTGATGCAAG
ACACAGAAGGTTGCACGTGGCAGCGTTCCATGTGACATTGCATGAAACCTGCAGTGTGGCCTGTGGGTTGTTGGCGT
GGGTCCTAGCACTACATGTGATGGGCAGTCCATATTTGAGACGTGGCAAATGTCCGTGTCATATGCCATGTGGCTGG
CCAGATGCCCTCATGCTGGTCCAAGATTTAATTTATTAATTTTTTAAACAAAGTATGTATTTATAGATTACCTTTCC
AGAGCAGCTAAACCAGGGAATGTCTTCCAAACAAGTGCGATGAGGAAAGCATACAAGCACTGATCAGCCCAGTGGCC
TGAGGTCCAGGGCCCTGGGCCAAGCCAGGGATTTAGCCACGAGGCTTGTGGATGACTATCCTCTCGCCTCAAGCCTC
AGTTTTCCCAATCTGGGAACTGGCTCACCCCTCCCGTAGCTTCCCAGGTCTTAGAGCCCAGGTCCAGGTAGCGTTAG
CCTGACCTTGGGGATCACAGGCCTGGGCTGTCCTGTGTAAGGGACAAAGCCAGATCTAAGGATGCAGCGGGTGGGGA
CTGCCAAGTTAGGCAAGGCCGCTGGACCCGGCCACTACCTCAGTGGATCTGACTGAACTCCCGGGAGCTCACAGTGT
TCATGTTGTTTCCAAGAAGGCCCAAGGATTGTGAGCCAAGTTTGATCAATAAATGTGAGTGATCTTCCGGCCTCTAA
AAAAAAAAAAAAAAAA

| | |
|---|---|
| SEQ ID NO: 7 | NLRC5 Protein Sequence |

MDPISRHLGTKNLWGWLVRLLCKHSEWLSAKVKFFLPNMDLGARNEASDPTQRVVLQLRKLRTQSQITWQAFIHCVC
MELDVPLDLEVLLLSTWGHGEGLPSQLEADEEHPPESQPHSGLKRPHQSCGPSPRPKQCRKQQRELAKRYLQLLRTF
AQQRYDSRSPGPGQPVACHRTYIPPILQWNRASVPFDTQEGTVAGGPKAEDGTDVSIRDLFSAKANKGPRVTVLLGK
AGMGKTTLAHRLCQEWADGQLERFQALFLFEFRQLNLITNFLMLPQLLFDLYLRPEAGPEAVFQYLEENANKILLIF
DGLDEVLHPGSSKEAADPEASASALTLFSRLCHGTLLPGCWVMTTSRPGKLPACLPTEVVTVSMWGFDGPRVEEYVS
RFFSDQPVQEAALAELRASWHLWSMCVVPALCQVACLCLHHLLPGRSPGQSAALLPTVTQSYVQMVLSLSPQGFLPA
ESLMGLGEVALWGLETGKVVFTAGDIPPPTMAFAAALGLLTSFCVYTEPGHQETGYVFTHLSLQQFLAALHLMASPK
VDRDTLAQHVTLNSRWVLRTKARLGLLDHHLPTFLAGLASCACHPFLTPLAQQEEVWVRARQAAVMQALEKLATRKL
TGPKLIELCHCVAETQKPELASLVAQSLPHHLSFRNFLLTYADLAALTNILGHRDAPIHLDFEGCPLEPHCPEALAG
CEQVENLSFKSRKCGDAFAEALSRSLPTMGSLKKLGLSGSRITARGISHLVRALPLCPQLEEVSFQDNQLKDGEVLN
IVEILPHLPQLRMLDLSRNSVSVSTLLSLTKVAVTYPTIRKLQVRETDLVFLLSPPTEMTTELQRDPDLQENASQRK
EAQRRSLELRLQKCQLSVYDVKLLLAQLRMGPQLDEVDLSGNQLEDEGCQLVAEAAPQLHIARKLDLSDNGLSVAGM
QRVLSAVRTCRTLAELHISLLHKTVVLMFAPEPEEQEGIQKRLTHCGLQAQHLEQLCKALGGSCHLKYLDLSGNALG
DEGVALLAQLLPGLGALQLLNLSENGLSLDAVFSLTQCFSTVRWLQRLDFSSESQHVILSGDSRGRHLLAGGSLPEF
QAGAQFLGFRQRRIPRSFCLKECQLEPPSLSRLCETLEKCPGPLEVELFCKVLSDQSLETLLHHLPRLPQLSLLQLS
QTGLSQRSPLLLADLFSLYPRVQKVDLRSLHHMTLHFRFSEEQEGGCCGRFTGCGLSQEHMEPLCWSLSKCEDLSQL
DLSANLLGDDGLRSLLECLPQVPISGSLDLSHNGISQESALRLVETLPSCPRVREASVNPGSKQTFWIHFSRKEEAR
KTLRLSECSFRPEHVPRLATGLSQALQLTELTLNQGCLGLEQLTILLGLLKWPAGLLTLRVEEPWVGRAGVLTLLEV
RAHASGNVTEISISETQEQLCMQLEFPHQENPEAVALRLAHCDLGTHHSLLVRELMETCARLRQLSLSQVKLCKASS
LLLQSLLLSLSELKNFRLTSSCVSSDGLAHLTFGLSHCHHLEELDLSNNQFGKEDTKVLMGALEGKCWLKRLDLSHL
PLSSSTLAALIQGLSHMSLLQSLRLSRSGVDDIGCCHLSEALRAATSLVELGLSHNQIGDAGAQHLAAILPGLPELR
KIDLSANGIGPAGGVRLAESLTLCEHLEELMLDYNALGDLTALGLARGLPQHLRVLHLRSSHLGPEGALSLGQALDG
CPYVEEINLAENSLAGGIPHFCQGLPMLRQIDLMSCEIDNQTAKPLAASFVLCPALEEIMLSWNLLGDEAAAELAQV
LPRMGRLKRVDLEKNRITAHGAWLLAEGLAQGSGIQVIRLWNNPIPQDTAQHLQSREPRLDFAFFDHQPQVPWDA

| | |
|---|---|
| SEQ ID NO: 8 | TAP Genomic Sequence |

GTCTGAGAAGAGCTTCACTCAGGAGCATCTGACCCACCAGGAGCCTGCAACATGGTCCAATAGCGCCCCTTATTAGC
CATGAGCTGCTGGTGGGTTCCCTCCTCAACAATGGTGCCTCCTTCCAGAAAGAGGATGTGATTGGCCTGCTCCACGG
AACTAAGACGCTGGGTGATGAGAAGCACAGACCGGGAGTACCGCTCAGGGCTTTCATACAGGAGCGACTCCACCTGA
GAAAAAAACACAGACTCTGTCAGAGCTGGGGGCCACTCCCGGAAGAGCTGGGACAGACCTCGCCAGGATCACTGCCA
CTTCTGCCAGGAACCCCAAAATCAAAGCTTCTCATTCTGAGTGCTTCTCTGTCAAACTTTTGATCTGTTAAGGACGG
TTTACATGAGGGGGCAAGAGCGTGTCCTATGGTGAAACTCATAAGTATGAAGGGTATTGAGTAGCCTCTCCTCTCTA
ATTTTTATATTCTCTTTCAAGGAGACATAAGTGAGTAGTAAAGAGAATGAATATTCGAGTCAGGCAGACTCGAATTT
GGGTCCAGGCTCTGCTATTCAACATTGAGCTGAATGCTATCGAGTGCGTTGTTCAGCCTCTCTTAGCCTGCATTTTA
GCATCTGTTCGATGAAGATAACAACAGCCAGCTCACAAGCATTCACGATGAATAATTAAATGAGAGAGTACATGGAA
AGGGCCTGTTAACATTTCTGGCACATGGTAAGATTTCAACTAATATTGGTATGATGGGATCTTTTCTTTTGTTTGGC
TTCACAGATTCAGAGTCTGAGGATCGTCTCTTTTAACTGACTCTAGGCATGTTGGGGAGAAGCGAAGGGGAACTGAG
AATTGCAAAGACTGGTTTGGATGATTATGATGTTAGTACAATAACAAAGGATGAGTGAAGGAAGGAGGACTGGGTGG
GTTACAGGCATTAAGAAGATGACTCTCTCACCCGTGCTTGACTGTTTGCATCCAGGGCACTGGTAGCATCATCCAGG
ATGAGTACCCGTGGTTTCCGGATCAAGGCTCGAGCCAAGGCCACTGCCTGCCGCTGACCCCCTGATAGCTGGCTCCC
AGCCTCACCTACCTCTGCAGAGACAAGTGCCCAGGTAAGAGCTGGATAAACACATGTGCATCCATGTGCTTGCATGC
ACGCGCGAGCGTGTGTGCACATGTGCACGCACGCACGCGCGTGCACACACACACACACACACACACACACACACTCG
GACTAACAGATACAGCTGGATAGGGAAGGTTCTGGGAAGGTGAAGGAGTTCTGAGGATATGAGGATGAAAGAGCCAT
AGAAACAAGCTCTTACAACTTCATACTGATGAATAAAGGCAAGACTATTGGATTTCAACAAAGGTAAAGATGTCTGA
GCCATAAAATAAAATTTAAAAAAAAAAAGAGTTCCTGCTGTGGCACAGTGGGTTAAGGATGCAACTGCAGGAGTTCC
TGACATGACTCAGTGGTTTATGAACCCAACTAGTATCCACGTGGACTCGGGTTAGATCCCTGGCCTTGCTCAGTGGG
TTAAGGATCCAGCATTGCCATGAGCTGTGGTGTAGGTCAGCAGCTGTAGCTCCGATTCGACCCCTAGCCTGGGAATG
TCCATATGCTGTGGTGCAGCTCCAAAAAAAAGCAAAAAAAAACAAAACAAAACAAAACCCGAATGCTGTGGCTCAGG
TCGCCTTGGAGGTGCAGTTCAATCCCTGGCCTGGTGCAGTGGGTTAAAGGATCTGGCGTTGCTGCAGCTGCTGCATA
GGTTGCATCCGAGGCTTGGATTCAGACTATGGGTGTGGCCATAAAAAACTAGCCCCCCCAAAAAAGATGCCTGGGTG
GTGATATGAGAGGAGAGAGCACCTGTGTCGTAGCCTTGCGGGAGCTTGGAGATGAAGCTATGGGCTCCGGACTCCAC
GGCGGCAGCTATGACTTCCTCCATTGCTGGCTTCTGGCTCAGGCCATAGGCAATGTTTTCTTGAAAACTTCTTCCAA
AGAGCTGTGGCTCTTGCCCCACCGCAGCCACCTGGGACAAAGCATGATGAGAGAACGAGGAACACAGGAGTATGATG
ATCTGGAGACTGAAGACTGAAAATCTTTATTGTGAACAAATCATGAAATCACACAGCCTCTCTCCTGAACACACCCC
CCGCCCCCCCAGGATCTCCTGTCATTCCCAGCACTCCTTTCAGAGTGCCCAGTGAGCATGGTCTTCTTACTCGCAGC
TCCCTGCCCTCCCCTGTGCCACCTTCTTGCTCACCTGTCTGTGCAGGTAGCGGTGCTCATATTCAGGAAGGGGCTTC
TCACCCAGCAGCACCTGCCCCTCCGTGGGCTGGTACAGGTTCTGCAGCAGGGCAGCCACGGTGCTCTTCCCAGACCC
ATTGGGCCCCACGAGGGCGGTCACCTCACCAGGACGTAGAGTGAACGTGAGGCCCTGGAGGCCAGAGAATCACACAC
TAAGAGGCAGATCAAGGCCCCTAACCTTAAGAGCGTCATGGACTTGGCCCATTGTTTTGTCAGTGTCTCACCCCAGA
GAAGAAAAGAGGAAAGTGGAGAAACACAGCAACTCCTACCCTCCCACATGCACAGACTTCTGCTCCTCAGCGATGCC
ACCTCCCCGTGGACTAGAGATGGAAGAAGAGACAAAGACCAGGGCAAAGACCATGCCGCACACTCAATCTCAGAGAC
CAGGAGAAAAAAAGAAAAAAAAAATCACATTTGAAATCACAAATGGAAAGAAAAAGGAGGAGTTCCTGTTGTGGCTC
AGGAGGTTAAGACCCTGACATAGTGTCCGTGAGGATACAGGTTCAATCCTTGGCTTCGCCCAGTGGGTTAAGGATCT
GGTGTGGCTGCAGCTGCCCCGTTCAGTCACAGAAGTGGCTCAGAGCCGGTGTTGCTGTGGCTGTGATGCAGGCGTTC
AGCTCCTGGCCCAGTGTGACCATTAAAAAAAGGAAGAAAAAAGGCAAGAAAAAGGAAAGATGGAAGACCAGATGGAT
ACACAGATTTTGCAGCAGTTCCTTAGGATATGACAGCCTTCTCCCTGAAAGCCTCCTTTCCTGTCCTCCCTGGAAAT
CCAAACTAGGTCTTGAGTTTGGGGCAATTTTATGGAACAGATGATGCTCATCTTTGCCTCTGAAGGGTAAAGAAGGA
TCTAGCTACACCTGATGTTAAGCAGACTGAAGGCAGGAAGACGATTCAGATCGAGCTGAGAGGAAGATTGGTGGAGT
GCAGGGGTTGGTGGGTTGTACCTGCAGCACTGGGACCTCTGGTCGGTTCGGGTAGGCAAAGGAGACATTCTGGAACT
TGACAAGCCCCTCTGACTTTAAGGAAGTCAACGATCCACTGGCCGGGCAGCGAGGGATTCGGTCCAGATACTCAAAT
ATTTCCTTTGAGGAGCCCACAGCCTTCTGTACCCTGGGGTAGGTGGACAGCAGTACCTGGAGGGGAGGTATGAATAG
TGAGATGGGAGGAGGTAGTGGGGGAGGGACCTAATCTGCCTGCCAGGATTATGTGATGTGAGAAGGGCAAAGCATGG
AAGGAAGGTGACTCAGATGGTGATGGGACAGGGGAGGGAAAAGCCCTGGGATGTGAGAATGGAAGGACCTCACCTGA
ACAGCTTCGGTGAACTGGATCTGGTAGAGAACAAATGTGACGAGGTTTCCGCTGCTTATAGCCCCACCTGCCACCAG
CTTCCCGCCAACATACAGGATTCCCACCTTCAGCAACATCCCTGAGATCTGTGGAGAGACCACACAGAAAAGGGACT
TTTGTAGAAAAATCTAGAGGGGCTGCAGAGAAGCAGAATCATTAGCATTAAGGAGATAAGAAGTTCTTGGAGTTCCC
GTCGTGGCTCAGTGGTTAACGAATCCAACTAGGAACCAGGAGGTTGCGGGTTCGATCTCTGGCCTCGCTCAGTGGGT
TAAGGATCGGGTGTTGCCATGAGCTGTGGTGTAGGTCAAAGATGTGGCTCGGATCTAGTGTTGCTGTGGCTGTAGCT
CTAGGGTAGGCTGGCAGCCGTAGCTCCGACTGGACCCCTTGCCAGGGAAACTCCAAATGCCTCAGGTACAGCCCTAA
AAAGCAAAAACAAACAAATAAACAAAAAAAAGGAATGAACCATAGCAATGCCACGGAGTCTCACTCAGTTATACAGA
AAAGAAGCCAATCGTTATTACCATCACCATTATCACCTTGTCTGGGAAGCATTTACTCTGCACAAAAGGCTTTCATG
AATGTAATGTCATCTAATAGTCGCATCAAAAGCCCCATAAACAAGGTTAGGTCACTGCCATTTTTAAAACTGAGAAA
ACAGTCTCAGAGAAGTGAAGTCACCAGCCCCTGGTCACAGAGCCGGAAAATGGCAGCATCGTGATAGGAACTTGATG
GCTGGTCGTGTTCGCTTTCGGTTACATCACAGGTGCCCCTCATCCTTGCTTCTGCTACTCCCAGGACTCTCACTAGC
ATCCATGTAGTGTCAGCATGAAACGGGACAGGGTGCCAGAATTTATAGTCCTCTGAGCACCCCCTTGAGGCAAAAGA
AGGCCTTGGAAAACACTTCCCTAAAGAGAGGGTTGGGTGGATTTTTGTGTACCGTAGTGAAAGGAAGCCATCTAGCA
CGCCTAAAAAGGGGGGAGGGGGTTAGGAACAGTGAGTAGGGTGACTGAGCCTCCGGTTGTTAGAATATGGCCACTGA
ACCAACCACTGGGCAGTGGAGGAAGAGTGTGGAGCAGGGTCATGGGAAAGGGAATGGCATTGAGGCATCTTGGGGAC
AAGGGACTAGGCAGTCATCTGCAGGTGCTCACACTGGTGGTCCAGAGGTCGACCGCATAGGCCAGGGCCTCCTTCTG
GTTGAGTGTCTTCATGTCCTGCAGCTTTTGCTTGAACTTCTGGGCCTCACCCTCTTCATTGGCAAAGCTCCGGACAG
TAGGCATAGCTGACAGAACCTCAATGGCCACCTGGCTTGACTTTGCCAGAGATTCCTGCACCTGTGCTGCCAGCACC
TGTGGAGACGTGGACCAGAGATGCCACACATGATTGTTGACAAACCATAGGGGACACTAGTACCTGAGTTATCCGAT
TAGAGTTTAAAGGTGAGACGTGGCAGAGGGAAGGCAAGGGGACAAAGGGACACAGCCAGGCCCCCAGATACTAAAGG
ATACAGAGAAGAGGAAAATGACTTAGAAGCGTCGTAGGGGAGCATATTCTTGAGATGGGTGATCATGTTCTTAAAGA
CAGATTGTGGGCAGGCATTAGAAGAGAAGACACAAGGGATGTGAAGATCAACACTGAGCAATCTGGGAACATGGACG
ACAGGGACAAGGAGTCCCACAAAGAGGAGAACCAGTGAAGGTGCCAGGAAAGGGATCTGAGCCCACCAAGTCTGGGA
TGAGGGTCAGTGTAGGTTGAGGCAACTCCCTAGACATACCTGGTGCCATTTCCCCAGCTTCTCAGGCAGAAGGAAAA
GCAGTGGCAAGGCGGCCAGGGTGACCATGGTGAGGGGAGGTGACCCCCAGAGCATGAGCCCTAAGAGACACAGTCCC
CGTGCGAGGTACCACAGCAAGAGGCTCAGCTCCGAACTCAGAGACACACTCACAGTGGATGTGTCCTCTGTTACCCG
AGATGTGATGGCACCTGCCAAGGGTTCAAGAGAAGAGAGTGGAGTGAACAGGAGGCTCAGAGTGATGGGAGCGACGA
GCAATGAGCCAGGTGCCACAGCGAAGGGCATCAACACAGTGTTCTAAGAAGGTCAGGAAAAGGAGTTCCCGTCGCGG
CGCAGTGGTTAACGAATCCGACTAGGAACCATGAGGTTGCGGGTTCGATCCCTGCCCTTGCTCAGTGGGTTAACGAT
CCGGCGTTGCTGTGAGCTGTGATGTAGGTTGCAGACTTGGCTCGGATCCGCGTTGCTGTGGCTCTGGCGTAGGCCGG
TGGCTACAGCTCCAATTCGACCCCTAGCCTGGGAACCTCCATATGCCGCGGGAGCGGCCCAAGAAATAGCGGGGAAA
AAAAAAAAAAAAAAGACAAAGAAGGTCAGGAAAACAAGGTCTGTGGTTGGGGGAGGACTGAAACATAATGCAAGAAA
AATGTGTTAGAGTGGAAAAGCCTGGCCAAAGACCTTCGTTTTAACTATAAAGAAATTGATGCCCAGAGTTCCCACTG
TGGCTCAGCGGTTAAGGACCTGACGCCGTCTCTGTGAGGTTGCAGGCTGGAACCCTGGCTTCGCTCAGTGGGTTAAG
GACCAGCTGTTGCCACAAGCTGTGGCGTAGGTCACAGATGCTGGATCAGGTGTTGCCATGACTGGCACAGGCCTCAC
CTGTAGCTCTGATTCAACCCCTGGCCCAGGAACTTCCATATGCCACAGGTGCAGTCATAAAAGAAAAAAAAATTTTT
AAAGAAATGGATGCCCATGTGAACTTCTGTTTCTCTGACAGGTGTCTGTTCCTTAAAGAACTTGTATATACCATGCT
CATAGGTAGGAAGAACTTAAGCTGGTCATACAAGAGCTGGAGAAAAATGGAGAGACTACTAGAGAGCAGTCCAGGAA
ACCACAGCAAGCACTGGATTGGGAATCAAGACATGGGTTCTGCTCTCAAGTTTGTCTTCATCCATGTGCATCCATGC
AAATGTTGGCATTTAGGTCTAGACCTCATTTCACTTCTCTGTAAAATGAGTCAGCTAGACTCTCTAATCTCAAAATT
TCCAGGTTTGAAATTCTACCTAAATACACTTATAGGGATAGTTTATGGAAAAATCTTGGGTGGAAACAGTAGGTTAA
TCATTTTTTTTTTTGTTTTATTGTGTTTTTGGTTTTGTCTTTTTTTTTTTTTTTTTTTTTTTTTTTTTTGCCCTTCC
CACAGCATGCAGAATTTCCCTGGCCAGATGGAACCTCGCCATAGAAGCAAACTGAGTCACAGCAGCGATCTGAGCCA
CAGCAGCCACAGAACTACAGCAGTGGCAACACCAGATCCTTAACCCGCTGAGCCACCGGCGAACTCCAACAGTAGGC
TTTTCTAAAGGTAAAGAGCATATCTTGCTCTTGAAGTACATCAAGAATAAAAAGGGACACCATTTGTGTGTGTGTGA
GAGAAAGATCAAGATTATAAGTAAAAGATGAAGTGTGGGGATACAAATAGAAAACAGACGGATAATGAAAGAGGTTC
ATAAGACACCTGTTTGATTCTTCTGAAAAAACTCTGTTTCTTGGCGCAGGACAGACCGAAACACCTCTCCCTGCAGG
TGGCTGTGCACGCGGCCCATGGTGCTGTTATAGATCCCGTCGCACACGAACTCCAGCACCGAGCTAGAGGGAGACAA
AGAAGGAGGGCCGGTCGGTCAGGGACCCCGTAGAAGTGCACTTTGGAGGGCGGCCCCAACTTCCAACTGCGCCCTTT
TCAGGGTCCCCCGTCCCCAGCCTTCCAAGCTCAGCAGTCAGACCTGGCTATGATGAGGATGGACATGAGAGTTAGGT
TCTGCGTGAAGGCAGCACCTGCCCCATCTCGTAGAATCCAGTCAGTGAGCCGGCCTGTGAAGAACGGAATGGCCATC
TCCCCTGGGGAGGGAGAGGAGAGATGGGCGGGTCAGAAAGAGCAAGTCTAAGCAGCCTAAGCAGCTCAGCTCTAACC
AGGCTGCACCTCCCGCCCATCCTCCCTTCACCCTTGCCCATTATCCTGCAGAAACAGCGCACACTCTCGGCACTGGA
ATGGGCCCCCGGGGAACTCGTAATCCTGTGGCCTCACCAGACCTTTAGAGGGTTAATTAAGAAGCCTAGGATGGTAG
GAGGAAAGAGCTCGCCCAAGGTGGCCAGTGAAGCAACACCTGAGCAGCACTGGAGTCCAGGACTCCTGACTCCCACC
CAGTCCAGGGCTCTTTCCTCTCCACCAAGTGGACCTGAGCGGGGTGGGCTTGCTCTTATCCACATTTCCGAGAACTC
ACACCTGTCTATCTCACTGACCGTTAGGCTTGATTCCTACCCAGCCCTCTAGCCTCCCTCTCCCTCCCCCCGCATCC
CCCTTACCAAGGCTGGAGAGGACCACCAGGGTCAGAAGGAGCCAGAGGTGGCGGATCTCTGAGCCCAGGCAGCCGAG
AAGCCGGCTCACTGTCACTCCAGAGCCTCTGTGACTTCCTTGCACCCAAAGGCTGCTAAGCTTATGCCACAGGGCGG
CCGCGGGCAATGCCGCCGCATAGCTGAGGGCGAAGGCATCGAGGCGACTCCCCCAGTGCAGTAGCCGCGTGCTGTCA
GCCGCTCCCGAGCCCAACTCTCGGAACAAGGCAAGTCCCGGCAGAGCCAAGCCCAGAGCCGCCGCCAGCGGCTCCAA
AGCTGCCAGCCATCCCCGAAGTCCTGTGCTTTTCTCCCGGAAGCCAACCGTCGCCCTGAGGACGCTGCGGGCCCCCA
ACCACAGCACAGCCCAACGGCTCAGGCCCACCACCCAGACCCGGAGCAGCGGCAGCGCTGGGGGCAGCAGCAGGGAG
GATATCCGGGGCAGCGCCGGCCGGAGCAGCACCCAGTCGGCGAGAAGCAGCAGCGCTGCCCCCAGCCAAGGGAGGGA
AGCTCGGGAGACGCAGAGACACCCGCAGGGAGCGGAGGACCCCGAGCTGGCCATTGGCCGTACGAGGTCGACCC

| | |
|---|---|
| SEQ ID NO: 9 | TAP1 cDNA Sequence |

GCCCTTGGGTCGACCTCGTACGCCAATGGCCAGCTCGGGGTCCTCCGCTCCCTGCGGGTGTCTCTGCGTCTCCCGAG
CTTCCCTCCCTTGGCTGGGGGCAGCGCTGCTGCTTCTCGCCGACTGGGTGCTGCTCCGGCCGGCGCTGCCCCGGATA
TCCTCCCTGCTGCTGCCCCCAGCGCTGCCGCTGCTCCGGGTCTGGGTGGTGGGCCTGAGCCGTTGGGCTGTGCTGTG
GTTGGGGGCCCGCAGCGTCCTCAGGGCGACGGTTGGCTTCCGGGAGAAAAGCACAGGACTTCGGGGATGGCTGGCAG
CTTTGGAGCCGCTGGCGGCGGCTCTGGGCTTGGCTCTGCCGGGACTTGCCTTGTTCCGAGAGTTGGGCTCGGGAGCG
GCTGACAGCACGCGGCTACTGCACTGGGGGAGTCGCCTCGATGCCTTCGCCCTCAGCTATGCAGCGGCATTGCCCGC
GGCCGCCCTGTGGCATAAGCTTAGCAGCCTTTGGGTGCAAGGAAGTCACAGAGGCTCTGGAGTGACAGTGAGCCGGC
TTCTCGGCTGCCTGGGCTCAGAGATCCGCCACCTCTGGCTCCTTCTGACCCTGGTGGTCCTCTCCAGCCTTGGGGAG
ATGGCCATTCCGTTCTTCACAGGCCGGCTCACTGACTGGATTCTACGAGATGGGGCAGGTGCTGCCTTCACGCAGAA
CCTAACTCTCATGTCCATCCTCATCATAGCCAGCTCGGTGCTGGAGTTCGTGTGCGACGGAATCTATAACAGCACCA
TGGGCCGCGTGCACAGCCACCTGCAGGGAGAGGTGTTTCGGTCTGTCCTGCGCCAAGAAACAGAGTTTTTTCAGAAG
AATCAAACAGGTACCATCACATCTCGGGTAACAGAGGACACATCCACTGTGAGTGTGTCTCTGAGTTCGGAGCTGAG
CCTCTTGCTGTGGTACCTCGCACGGGGACTGTGTCTCTTAGGGCTCATGCTCTGGGGGTCACCTCCCCTCACCATGG
TCACCCTGGCCGCCTTGCCACTGCTTTTCCTTCTGCCTGAGAAGCTGGGGAAATGGCACCAGGTGCTGGCAGCACAG
GTGCAGGAATCTCTGGCAAAGTCAAGCCAGGTGGCCATTGAGGTTCTGTCAGCTATGCCTACTGTCCGGAGCTTTGC
CAATGAAGAGGGTGAGGCCCAGAAATTCAAGCAAAAGCTGCAGGACATGAAGACACTCAACCAGAAGGAGGCCCTGG
CCTATGCGGTCGACCTCTGGACCACCAGTATCTCAGGGATGTTGCTGAAGGTGGGAATCCTGTATGTTGGCGGGAAG
CTGGTGGCAGGTGGGGCTATAAGCAGCGGAAACCTCGTCACATTTGTTCTCTACCAGATCCAGTTCACCGAAGCTGT
TCAGGTACTGCTGTCCACCTACCCCAGGGTACAGAAGGCTGTGGGCTCCTCAAAGGAAATATTTGAGTATCTGGACC
GAATCCCTCGCTGCCCGGCCAGTGGATCGTTGACTTCCTTAAAGTCAGAGGGGCTTGTCAAGTTCCAGAATGTCTCC
TTTGCCTACCCGAACCGACCAGAGGTCCCAGTGCTGCAGGGCCTCACGTTCACTCTACGTCCTGGTGAGGTGACCGC
CCTCGTGGGGCCCAATGGGTCTGGGAAGAGCACCGTGGCTGCCCTGCTGCAGAACCTGTACCAGCCCACGGAGGGGC
AGGTGCTGCTGGGTGAGAAGCCCCTTCCTGAATATGAGCACCGCTACCTGCACAGACAGGTGGCTGCGGTGGGGCAA
GAGCCACAGCTCTTTGGAAGAAGTTTTCAAGAAAACATTGCCTATGGCCTGAGCCAGAAGCCAGCAATGGAGGAAGT
CATAGCTGCCGCCATGGAGTCCGGAGCCCATAGCTTCATCTCCAAGCTCCCGCAAGGCTACGACACAGAGGTAGGTG
AGGCTGGGAGCCAGCTATCAGGGGGTCAGCGACAGGCAGTGGCCTTGGCTCGAGCCTTGATCCGGAAACCACGGGTA
CTCATCCTGGATGATGCTACCAGTGCCCTGGATGCAAACAGTCAAGCACGGGTGGAGTCGCTCCTGTATGAAAGCCC
TGAGCGGTACTCCCGGTCTGTGCTTCTCATCACCCAGCGTCTTAGTTCCGTGGAGCAGGCCAATCACATCCTCTTTC
TGGAAGGAGGCACCATTGTTGAGGAGGGAACCCACCAGCAGCTCATGGCTAATAAGGGGCGCTATTGGACCATGTTG
CAGGCTCCTGGTGGGTCAGATGCTCCTGAGTGAAGCTCTTCTCAGAC

| | |
|---|---|
| SEQ ID NO: 10 | TAP1 Protein Sequence |

MASSGSSAPCGCLCVSRASLPWLGAALLLLADWVLLRPALPRISSLLLPPALPLLRVWVVGLSRWAVLWLGARSVLR
ATVGFREKSTGLRGWLAALEPLAAALGLALPGLALFRELGSGAADSTRLLHWGSRLDAFALSYAAALPAAALWHKLS
SLWVQGSHRGSGVTVSRLLGCLGSEIRHLWLLLTLVVLSSLGEMAIPFFTGRLTDWILRDGAGAAFTQNLTLMSILI
IASSVLEFVCDGIYNSTMGRVHSHLQGEVFRSVLRQETEFFQKNQTGTITSRVTEDTSTVSVSLSSELSLLLWYLAR
GLCLLGLMLWGSPPLTMVTLAALPLLFLLPEKLGKWHQVLAAQVQESLAKSSQVAIEVLSAMPTVRSFANEEGEAQK
FKQKLQDMKTLNQKEALAYAVDLWTTSISGMLLKVGILYVGGKLVAGGAISSGNLVTFVLYQIQFTEAVQVLLSTYP
RVQKAVGSSKEIFEYLDRIPRCPASGSLTSLKSEGLVKFQNVSFAYPNRPEVPVLQGLTFTLRPGEVTALVGPNGSG
KSTVAALLQNLYQPTEGQVLLGEKPLPEYEHRYLHRQVAAVGQEPQLFGRSFQENIAYGLSQKPAMEEVIAAAMESG
AHSFISKLPQGYDTEVGEAGSQLSGGQRQAVALARALIRKPRVLILDDATSALDANSQARVESLLYESPERYSRSVL
LITQRLSSVEQANHILFLEGGTIVEEGTHQQLMANKGRYWTMLQAPGGSDAPE

| | |
|---|---|
| SEQ ID NO: 11 | GGTA1 Genomic Sequence |

ACTGAGAAAATAATTTATTTAATTTTAAATCAGGAATTTTTATTTTTTAATATTGAACTATTAATAAGATCTTGAAT
TTGTCCATTTGAAATTTAAATTTAAATGATTTTTTTTTAAAAAATCAAGATTCCTTCAAAAGGAAATATCAGTCCTT
TTCTTTAATCTTTGAGAACGAATCATTTCTGTAGTTTGGAACTTGCACCATGAAGTCTCTGCACTCCAGAATGGATT
CCATAAACTTGCGTTATAGAGAAACAAGAGTCCTAATTGACTTGTGATTTCCTTTTTCTTTTACAAGACTACTTCTC
CAGGATTTTTGTTGAGTTATTTTGTTGGGTTATTTTGTTGAGTTATTTTGCTGGGTTGCAAAAATTTTTAGCAAGAA
TTGAAGAGTAGGAGGCCCAGGGAAACAGTAGAGAAAATGTAGGTTTCATTTTATCAAAGAAGCCCATCGTGCTGAAC
ATCAAGTCAGTGCAATGGCTCTTCAAGTAAATCATTTGAAAATGGACACAAATGACCTAAACTGGAACACAAGCAAA
AGTATATCACATACCTGCAGATGTAAATATTGCCTCCTAACTTCCTTTACACCAAACTGCTTAACTTTAAATTACAT
GTAAGATCTCATAGCTTTTCTTAGAGAAAGGGATTGAAAAGCTGTTTAGTCATGAGGACTGGGTCTCCCATTGCCAT
CCTCTCTACTTTGATATAAAATCAATTAACCACTTTATTAAACATGTCCGGCAGTTACACTTCAGTAGTGCAGCTGG
GGCAGGGGAAATGAGAGGTTCCCTGATAAGCAGGCTTTTCCTCTAGTCCACTCCTTGACGGTGGCTCTCAAGTTGCC
CATGATGGGCTGAGGGACTCTGAGAGTTAGAGCAGGTGGCAGCAGGACTTGCTGATGCCTGATTGTCATGAAGCCAA
GATCTAGGAAGTCACTTCAACCCACTGTAGGCCTCTGTCCACTCTGACATCATCCACTTCCTCTGAGCAAGGATTTG
TAGACACAAATTCCAGAGTCTGGCAGACTGAATATGACTTGGCCAAAGCAAGAAGCATCTTCTAAGACAGTGCTGCT
CTAGTTGTCATATGGTTGAGGAGGCTGGAGCCACTCTCATTGCCTCCCATTCAGTGCCTGGATCCAAGCTGTATGTA
CATGCCAACTCCATGCCCTGTGTCTCTTAGAAATGGCATTGCCCCACAGTGATCAGCCCCCTCTCTTTCCAATCTGT
CTTCGCTATTTCATGGCAAACTTACTTAGAAGCTGTGCTTTTATTTCGTGCTGAGCTCCCATTGGTTCATTCGGATT
CCCTGTAACTCCCAACATTCACCATTGGGAATCTTGATCAGTATCTGCGCAGAAGCCAAACAAAACCCTGATGCGAA
AAGGACATGGACTTCAAATAACCTGAAGTCCTCTGCTGTTGAAATCATCTGAGGATTGCTAAGGTAGACTCTGATCT
CCTGCTGCAAAGCAACTCTGTTGCTTTAGACTTAGCAGAGACAGGAAGACGCTAAAATCAAGAGGACGACCCCTCCC
AATCTTATTTTGTTGCCAAACACTTCCCTTTGCATACTTTTCTCCAGTATGACATGTAGAGTGTCTCTGACTTTTTC
TTTGCCTATGACAATTTTTTTTTTTGGTTCAGTTAATAGTATATACCCCCTCAACCCAGAACAGATAAGAAATCATT
GGGAATTTACATCTGATTACTACAGAGTCATTCTCCCATTTGACAAGGCTCAAAGTTGCAAGGAAGAATAATATGTA
CTTACTGTGTTGGTATTTTGTTAGTATTTTTTTAAAAGTTAAAATTAAGTGCTACTTCTCTGAGGAAGTAGCCAGAG
TAATACTCTTTCAAATTCAGAAAACTGCTGGCACAATTTAAAGTCAGATGTTATTTCTAACCAAATTATACTCTTTT
TTCTGCCAAGCTATCTTGACAATCCTAATATCCACAGACATGCCTATATGATAATCCCAGCAGTATTCTGGGGATAA
GATTTTAGTGGGTTTGTTGAGAAGGAAATACTTGTTTAGATGGCTTTCATCATGCCACTCGGCTTCTATGTCATTTT
CCTTGTCCTGGAGGATTCCCTTGAAGCACTCCTGAGTGATGTTTAGAACCTGAGTGGGTGTTCCCCCAAAAATGGCT
GCGTGGTAATAAAAATCCCCCTGGCCAAACGGAATGTAGGCTGCGGACTCCTTCCGCCTCTCGTAGGTGAACTCGTC
AGGATGTGCCTTGTACCACCAGGCCTGTAGCTGAGCCACCGACTGGCCCAGGGTCTCCACCCCAAAGTTGTTTTGGA
AGACCTGATCCACGTCCATGCAGAAGAGGAAGTCCACCTCGTGCTGGATGTGGGCCAGGATGTGCTCCCCGATGGTC
TTCATGCGCATCATGCTGATGTCTTGCCACCTCTTCTCGGACTTGATCTCAAACACTTTAAAGGAACGCAGAGGACC
CAGCTCTATCAAAGGCATCCTGGAGATATCATCCACCATGATGTAAAAGATGACTTTGTGGCCAACCATGAAGTATG
TATTTGCAGATATTAAGAACTCCTCCAAGTAATGCTCAATGTATCTGAAATAAAGAAGAATGGGGTAAATGTAACCT
CTGGGATTTCTAGAGGAGACAATATGCTATTATCATCTAGTCTGTATTTTGCAGTTTAGGAAAGGAATGATTTTTCC
CCATCCTGGATGAGAGACGTCTGTTGCTGTAACATTCCCAGCTACTCTCCACCATTCAGTCATTCAGCTTTGGGGAG
GTGGAGTGGCTTACCTGACTGGTGATTCTGGCAGGGTGGCTGGGCATGCTCAGCCCTGCTCCTTCCTCTCTCACTCT
TGGAAGCCAACCAGGCAGAGAGAACATGTGTTTTCAGCTGCTCTGGGCCTTGCAGTGGTACCTTAGTGGCACAGGCC
CTGCTCCCACATCCAGAGGCCTGCAGTTACTTGTGCTGTATGTGCCTGGATGCCTAAGTCTTTCTAATTCTGTGGTT
CAAGATTTGGAAGCCCAGGGCCTGCAGTTATAAGCCACATACTCCAACACCAGCTTTAACTGTAATGAAGGTGATAA
CTCATTACCATCTGCCTTAATTAGTCTTTATCCCCTTGTCCTTATCAATCAGTTCAGATGCTAGTTCTTCCTTTTTT
CCTGCATTATTCAGATATAACTGACATATATCATTGTGTAAGTTTAAGGTGTGCAAAGTGTTGATGTGATGCACTTA
TTTTTAATTTTTATTTTTTGTCTTTTTAGGGCCACATCCGCAGCATATGGAGGTTCCCAGACTAGGGGTCTAATTGC
AGTTGCAGCTGCTGGCCCATGCCACAGCCACAGCAACACCAGATCTGAGCTTTGTCTATGACCTACACCGCAGCTGG
TGGCAATGCTTGATCCTTTAACCCACTGAGCAAGGCCAGGGATCGAACCCAAATCCTCATGGTTACTAGTCAGATTC
TTAACCCACTGAGTGACAACGGAAACTCCCTGGTACACTCATATATTAGAAATGATTACCACTGTGGCATTACTTGA
CACCTTCATCATATCACATAATTACCATTTTTTTGTGGCAAGAAGACTTAGGACTTATTCTCTGACCAACCTTAAAG
TATATATTACAGTATGATTAAAAACAATCACCATGCTGTACATTAGATCCCAGAGCTTATTCATCTTATAACTGCAA
GTTTGTACCCTTTGATTACCATCAGGGGGCACTAGTTCTTAGCTCTTCCTCAAAAACCCCAGCCTATATTCCAATAC
TTTTACTGACCTACCAGATGCAAGCGTGATGTGCAAGGGTCATTAAGCCTAACCATCGCCACTCTCTTATCCTTCTC
TGGGACCCAAACAATGGATTATGGAATATGGATATTCTTCCATCTTACTGATTTACCCTGTGAGTTTCCCGCTGGTC
ACCCCAAACACCAGCCCATTATCCAGACACCATCATTATAAAACCCATCCAAATATGAGAGCAAACGACCTCTGATT
CAACCTTACTTTAACTATCTCGTTTCATTTAAAAAAATAGATTTTAGTTTTTAGAACATGTTTAGGCTCACAGCAAA
ATTGAGCTGAAAGTGCAGAATTCCCCCCGCTCCCCCCACTCCCACTCCCAGCTTCTCCCACCATCAACATCCAGCAC
CAGGGTAGCACGTGTTGCAACTGATGAAACTACACTGACACATCATTATCACACCAAGCCCGTAGTTTACACTAAGG
TTCACTCTTGGTGGCAGACTTTCTATGAATCTGAACAAATGTAAAATGACATTTATCTATCACTATGTATGGTACCA
TACAGAGTATTTTCACTGCCCTAAAAAATCCTGTGTTCTGTCTATTCATCCATTCTCCCACACCATCGCCTGGCATC
TACTGATATTTTTACTGTCTCCATGGATCAGTACCTTTGACCTTTTCCAGAATGTCATATAGTTGGAACCATATAGT
AGGTAGTCTTTGCAGATGGTTTCTTGGTAACGAACATTTGAGGTTCCTCCATGTCTTTTCATGGATTGATTTTTTTT
TTTAAAGCACTGCTAATACTCCACTGTCTGAATGTGCTACAATTTATCAATTAATTTGCCTACTAAAGGACCTGTTA
CTTCCAAGTTTTGGGCAATTATGAATAAAAGTGCTATAAACGGAGTTCCTTTCGTGGCTCAGTGGTCAACAAACCCA
CCTAGTTGCAGGTTCAATCCCTGGCCTCGCTCAGGGGGTTAAGGATCCAGTGTGGCCATGAGCTGTGGTGTAGGTCG
CAGATGTGGCTCAGATCTCGGGTTACTGTGGCTGTGGCATAGGCCGGCAGCTGTAGCTCTGATTCAACCCTTAGCCT
GGGAACCTCCATATGCCGCAGGTGTGGCCCAAAAAAAACAAAAAAAGAAAAAACCAAAACCCACCCCCCCCAAAAAA
AAATACCTGCTATAAACATCTGTATGCAAGTTTTTGTGTAGACATAAAGTTTCAGCTTTTGAGGGTAAATACTAAGG
TGTGCCATCGCTGGATTGTATGGTAAGAGTATGTTTAGTTTTGTAAGAATCTGCCAAACTGTCTTACAAATTGGTTG
TATCATTTCGCATTGCCAGCAGCAGTGAATAAGCTTTCCTATCGCTCTACATTTTCATCAGCAGCTGGTATTGTCAG
TGTTTGGGATTTGGGTCATTCTAATAGATGTGTAGTGGTATTTTAGCTATTTACCTATTCATTCAAAAACCATCATG
TTCAGGAAGAAAAGGAAAGGGGGGAGTTCCCATTGTGGCAGTGGCACAGTGGGTTAAAGATCCAGTGTTGCTGCAGC
TATGGAGAAGGTCACAGCTGTGGCTCAGAACTTCCATACGCCACAGGTGCAGCTGAAAAAGAAAAAGAGAAAAAAAA
AAACCCATCACATTCCTGTCTTCTGTAAGCCAAGATACAGGCTATTCTGTGAAGCCATGGGGATGATAGAGAAGGGA
AGAAGTAGTTGGCTGGCTTAACACAACCCACGTCACCACCCAGACTCATGCCCAGTGACTGTGCACTGAATTTAATT
TGTTGATCACATTATCAGCCAATGATGACATTTTGTAATAATGACTGGCACTTCCTTTTGTTTTTTGGTTGCTGCTT
GGATTCCCTTTGATTACTACAAACATAAACTGTGCTTTCAATGCTGGTCTCTGGAAACCCCAGGTTTATAGTATTGA
TTCTTTAAACGGAGAGAATATCTCAGCAATACAAGGAGGGACTTCAACATGGCTCTGGGGCTAATGGCCAGGAAATT
CTTCTGCACTCTGGAACTTTAAGAAAAAATCTATTGTGCCCTGAAGCTTGGGAGGTGATCCTAGGGGCGAGGGAGGA
AACCTTTGTGAGGTTTAACATTGTTTAGAGATTAAAGCGCTGCAGTTGGTGCTGTGCACTGTCATTTGAAAATAAAC
CAAACATCACACCTCCTAAAAGTCCAAATCCACTCTTGGGAGGATTTATTGCTGCTGAGTACAAACAGTCCTCACTC
GCCTCAGAGCAGAGTGCGCGGGTTTCACCAGGACATGCCAAGTACAGTTTAGTTCTCTAAAGCTGCAACAAGATGGC
TAGAGCCAATGTGGAGCCGTTCTTTTTGGAAACACCAAGGTTAAATCAATCTGCAGTATGGCTGGCTGGTCTCCTCT
TATACCAAAGGATTAGGTGAGCTGGGAATCTTTCCCAACTCCTAACAGAACATATTCTTCTAGTCGAAAGGTCAAAA
CTCCAGAGTCACCCTTCTCTATTAGAGATGCCACCCAGGCCCCTGGGATCAGTACATTCAGGGACATTAGGACTTGA
TTAGTACAGTGACAGTGATACCTTCTGGGCTCTAGGTTGGAGAAGGTCTCAGGAGGACGCTTAAATCTTCACTCAGA
TCAACCTTGACCTTCACTTCTCTTTGTACAGGCAACAGGTCAACTAACTTCTTTTCTTTTCTTTTCTTTTCTTTCTT
TCTTTCTTTCTTTCTTTCTTTCTTTCTTTCTTTCTTTCTTTCTTTCTTTCTTTCTTTCTTTCTTTCTTTCCTTCCTT
CCTTCCTTCCTTCCTTCCTTCCTTCCTTCCTTCCTTCCTTCTTTCTTTCTTTCTTCCTTTCTTTCTTTCTTTCTTCC
TTTCTTTCTTCCTTTCTTCCTTTCTCCCTTTCTCTCTTTCTCTCTTTCTCTTTCTCTTTCCCTTCCTTCCTTTTCTT
TCTTCCTTCCTTCCTTCCTTTCCTGCTTTTTTAGGGCTGCACCCTCCCAGGCTAGGGGTCCAATCGAAGCTGTGATG
ATGGCCTGCGTCAGAGCCACAGCAATGCGGGATTCGAAATGCATCTGTGACCACACCANNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNN
NNNNTTTCCTTTCTTCTCTTTCGGATTTTTTTTTAAGTTTGGTGAAAGTATAGTGTCTTACAATGTTGTGATAATTT
TTCTGTATACAAAGTGATTTCAGTTTCTTTGTGGCTTCAGAAAAGGTACAGATGGAAAGGCCCATGGATGTGGGGGA
GGGAAGGGGCACGGAGGTGAACAGGAAAATTGAACTTTTGCTTTTGTTTTGGAAAAAAAGGGGGGGGGATTCTCTAA
AAAAGAAAACTGGGTTATATTTTAAACGAACATTACAGCTACTACTTTTAAGTAAGAATGTTTACAGTTTGGGGAGA
AAAGTTCCAAACAAGGAAACGGGGGCTGAAACAGGAACCTATCCAACCTCTGGAAGAGGAAGTTCTGAGCAGCCTAA
TCTCCCCGGGCCAAACCCTCCAGGAGGAATAGGCAGAAGGCACAGAGGAGTGGTCAGCCATGCGGACGTGGAAAACC
ACTCCACTTAGGACACTTCTGTCTTTGGTCCTTGGTCTGGGGTCTCGAGAGCATAGGAGAAACGACGCACACACAGG
CCATCTAACAATTGCCATTTTTGGAATTTCCACAGAGGGCCGTGGAGGTCAGGGCGGAGGTGGCTGTGGGTGTACTG
TCGACTCTGGGTGCAGTGGGTATAGCAGATCTTCTTCCCTGCAACCCAAGCCCCTCACCCTGAGGTGGGAAAGAGTT
GACCCTCTGACTAGTTTTATTCTTAGCCTTTGGGGACCTCAGCAGAAGGGAGTCTAAAATGGCCCTGTGACACCATT
CTCCTCTCCACTAATTCAGACATGACATGAACAGCCTCTGTAAACCCAGGGGCCCCTCACCCATCCTCTGATAGTGG
AAGGGGAAAAACTCAAGGCCAGTTTTATTAGCAACACCTACCTTCCGACAGCAAAAACCGTCAAGCCCACGGTAATT
TTCTGTTTGGCATAATAATTATCTAAGACGGCTCTGTTGTAAGTGCCTTCCCATACCACTGGAGCCTTCCATCTGGT
TATGGTCACGACCTCTGGGCGTTTCCTGGTGACAAAACATAGAGTCAGGATGGCTTTGCTAAGGTACGACAGTCTGG
GGGAACATGGGTCAGTCATGGCTTGTGGTGACTGGCCTTGAATCCTGACTGTATTTTAGCCCCAGTCAGCTGGTGGT
GTGACATTGCAGCATCTTCTGGGGGAGGGACAGGAGGCTCTGGCCCAGGTGCCTCTGCGGGCTGCCCTGGTGGCCCC
TTTGGGGATCGTACCTGTACAACGTGTATGTACCTTCCGTCCCCCTGTTCTGCTGTCCTCGTCCTCAATCTTCCTTC
CAAACCCCTTCGCCTATCTCCCCAGGCCCTTCCTAAGCTGCCAGCGACATCTTTGGGTGTTGCTTATCCCAGTGGGT
GCCACCTGACCCTGAGAAAGCCCTATGGCTTGACTAGCGGGATGAGAGAGTGACATTTGAGCTGAAAGAGGAAGAAG
CTGTCTCAGTTTGCCTTCTGCCAGAAAGCAATTTCTGGGTAGGAACCTGGTTATCGGACAAAAAGGGCCCCAGACTA
AGGGGACCTGGTGTTGTGGTTCATTTTACGAAGAAGGAGACAGTCACCCAGAAAAGAAGGGACCCGGCGGGCTAACT
GTGGCCATGGGTGACACACAGGGCTCGGGCTCAGACCTCTCTCAGATCATGTCACCTCTTGACTAGAAGCACAAAAG
CGGGAGGGGAGGGGGCATGTTCTCTGCACCCAGAACACTTGAAAGGGACTTAGCAAAGCCAACACAAACACAGGAAG
CCACGGAAGAGCAACGGACAAATTGTAAAGAGTAAATGCGGGAAGTCTGGGTAGCAGCTGGGGCCCCCCAGAGGCAG
GAGGGAGCTGAGAAGACTTGGCTCAAACCCCATTTGCTCTGGAAGTGGCTGCACTTCCCCGTCGGAAACAGACTGAA
ACGTGGTCATTTAGATTCAACCCCCAACACAACATGAGAGGGCCTGGCCCCTGCTAGCTGTGTGCTTGTATTTCAGC
CACTGCAGGGAGAAGGCCAGTGGTTGGGGCAACGTCTTGGGGGTCCCATCGGGCCCCTGCTGGCTGCCTGGGTATGG
CCCTGGTGAGGCTGTCTAGGAGATGTTAGCCCAGCGAGAACATACCCCCACCCTCATACGCGGGTGGAGGAAGGGTT
TTCACAAACCTGCCCCTCCCCCATGGGAGAAACCATGTTTCCCTGCGAGATTGGGCAAGGCTGGGTCACCCCCACTT
CTTGCTCATGCCTTCTGTCCCTCGTCACCAAGCTCTGCACCCGTATTCTGGAGCTGCCTCTGCCCTCCCACCCCCAC
CCCATGCCCTGCTTCAAGCCTGCTTCCTTCCTCCCCTAAGAGTAATTCTGCAGAGATGGAGGGGACATGGCTAGGCT
GCTCAAACCCCACACCCCCAGCTCTGCCTTCACACCCCAGGTATGACCGCCCCTTGGGGACACCTGCTCTTGGTTTC
CAACAATCATGAAAGAAGCTGTTTTGGACTCTGTACCAACTTGTGCCAGGTACTTTCACATACACTTTCTCTCATTT
AGTCCTTGCAAAAGCTTGGCCATGTAGTATGCTCAATGTACAGATATGAAAATCAAGGCTCAGGAAGGCTTGTTAAC
TTGACCAAGGCCAAACAGCAGATGATGGTAACTAACACACACTGGCTCCTTCCTATGGGACCAGGCACAGTGCCAAG
AGCTTCACCCTTTTGTGGGGGTGGGGTTGCTATATTTTGATTCCCATTTTATCTGTGAGGAAACTGTAGCACAGAGT
GGTGAAATAACTTGTCTGAGGTCACACAGCTAGTAAGGAGCCAAGCTGGGATTTGAACCCAGATAGTCTGACTGTGG
TCTGTGCTCTGAACCACTACCCTCTATGGCTTCTTGGCTATTTACTTGCTGTACCAATGAACTGGAGTTAAAACCCA
GGTATGTCATCATTTCCACTCATTTGAGCTACTTCAGCATTTTTATCAGGGCAGAATAAAAAAAAATGATGAGCTTT
TTTTTTGTTTGTTTTGTTTTGTTTTTAGAAACTTATGTGATGCTTTTCTCACATAAAAGCCCCAGCTTTGTTGAATG
ACTGGATTTCAAACCAAAAAAACCACACACACACACACACACACACACACACACACACACACACACACACAGCTTAG
GCTTATCATTCTATAACCGTTTCCCATGCACTGTCACTTCATTCATTCCTGTCCTTAGTGTAGCCTGTCAAGGATCT
CTTAGCAGTTCAGACCCCAGCCTATCAGTTAAGCCATGCAGCTGTGTGTGAGCTGAACATCTGGCAAGCAGGCAATA
TTATCTTTAAGCAAAGAAAAGGAAGAGAAAGAGAAGGAGGAAGAGGAGGAAAGGAAGGTATTCTTATTTACTAGTCG
CAAGCACTGGGGTTAAGTACCGGACTTTTATTCTCTCATTGAATCCTTACAACCACGTTCAAGAGTGGGTGCTATCA
TCACCTCCATTTCACAAATAAAGAAAGTCGGGGGTGAGAGAGAAGGAAACTATGTTTTTAGCCATTCAACCAATAGG
AGGGGCCACACCAGGGCATCACCTCCTCGATGCACATCTGCCAAGTCCCTGCTCCATCTGCCGGGGCCCAGGGCTAA
AGACGGAGATCAGACCCATCCTACCCCTTGAGAACTTCCCATCCCTGACAGGTGGTCAGCCTGCCGCACACTCCTCA
GCCGCACAACCCCTCAGACTACACCTTCTAGAAAGACCGATTCAGAACACCAGTGTCCAGTTTGGTTACTTGGCTGG
GAAGATTCCTTTTAAGCAGGGGGGAGAAAAAGTAGCAATATTAAAAATTAACGTCGAATTAAAAATTAAAATGCTCT
ATTTCCCAGCTGTTAATTATTAAATTCCACTGGCAATTCCAACATGTCAGCAACCCTGACTAGGAAGCCATATGACA
GGCTGAAAACACTGGCCGTGGGCAGGAGGAGGAGGTGGGAGGATGATTGAGATCAGCTTCCTGGATGAACCTCTGCT
CAAACCCCACCCCCACCCCGGCCCACAGAAAAAGAAGAAGTAACAGCAGGCAGGCCAAGTATGTGTAAGAGCAAGAG
CTGCCCAACGTCATCAAGAGAGGGCTCGAAAAGGAGGGAAAAGTCCAGGAAACACTGGAAACTGCTCAGTTTTTTAA
GCCGGGCACCCACTGCGTTACTTCGGCATGTGGGGTTCCACCAGTGCAAACCAAAGACTTCCACAAAATAAAAGGGT
CTCCAAAATCCAAACGCACCACCTACCTAGGTAGTTGGTAGCTTTTCAATTTTATGTACTTATTTATGGGTACACTG
TGGTCCTGAAGGGCTGGGCAGAGGAAGTGTTAAAATTCTATGAATCATACAGCAGGTGGAAAAAAATGAGGAATGCA
ACAATGTGTTACTTACTGGATTCCTTCCAGGCAGCAGGACGTACACAGTGATCCAGCAAAGAGCTAATGATGCCATG
GACAAGGGTGATGGAGAGAGGGAGATGACGTGGGAAGAATGAACAGAACATGTAGATGAATTAGACTGTGGGCTGGA
TGAAGGAAGGATGAACAGTGAATCATGGAGGTCTCCTGACTCTTGCTTGAGATGGGAAATGAGAAGAATGAGGGTGG
GGTGGAATCAAAAACTCCCTCTGGGAGTTCCCGTCATGGCTCAGTGGGAACAAATCTGACTAGCATCCATGAGGATG
CAGGTTCGACCCCTGGCCTTGCTCAGTGGGTTAAGGATCTGGCGTTACCGTGAGCTGTGGTGTAGGTCACAGACACG
GCTTGGATCTGGTGTTGCTGTGGCTACAGTGCAGGCCGGCAGCTAGAGCTCCAATTCAACCCCTAGCCTGGGAAACT
CCCTATGCCTCAGGTACGGCCTAAAAAGACAAAAAACAAAAAAACAAACAAAAAAACCCAAACTCCATCTGAGTCAT
GCGAGACCTGCAGTGATGTCAGGCAAGAGTTAGACACAACTGGGTGCTCAGAGAAAACCTTTGGGCTAAAGATATAA
ATGCAGTAGTCATTGTCCCATGAATGGTATCTAATGCCACAGAAATGGATGAAGACAGTGTATAAAGAAAAGAGATG
AGGATAATGGACTCAACCTCCAGAAACTCTAACACTTCCTGGCTGAGAAGAGGGAGGGGCCCCAATCAAGGAGACTG
ACAAGGGAGCTGGAGAAGTCGGAGGAAAACTAAGAGGATGTGGTGCTACAGAGGCTGAGAGATCTTGATGTAAAAAT
GTATACAGAATACACTTAATATGTTTCAGGTAGAATACAGAGGACACATTTCTATAAATATATCTATAATATATTTC
TATAAATATATTAATTCAGTGGCTCATCTTTCCTGCATTTATGCAAGCAATTTACTTTGGTGCCCTGAGAAGGCTTA
GATTAGTGCTACTACATATCAATATTCTTTAAATATCTGCTCAGCATTCATTTGGAGGAGAAACTGAGCCATGCATG
GGGGAAAGTGGAAAGAGTGACAGTGGGTGGCTGTGGTCTTTCACCTCTGACCCCAGTGATTCAGCCCTGGCTCCACC
TCTCAAGTCCCACTCAGTAAAGCACAAGTACCACGGTCAGTGTGCCACTCTCTCTTGAAGGGAGCTTGGTGACTGTC
TCTAGCTGATCTATCTGGCCCCTGGGGAGTCTCACACCTCCCCACATGCACACACATCTAAGGGGCTTATCAAAGCT
CTGGTGGGAGTTCCCGTCATGGCACAGCAGACATGAATCCAACTAGTATCCATGAGGTCGCCAGTTCGATCCCTGGC
CTCACTCAGTGGGTTGGGGATCCTGCGTTGCTGTGGCTGTGGTGTAGGCCAGCTGCTGCAGCTCCGATTAGACCCCT
AGCCTGGGAACTTCCATATGCTGCAGGTGTGCCCCCTCAAAAGAAAAAAAAGTTATAGTGCTTCCACATTCTTCCAC
TTCCAGGAGTAGCTTAGCATTCCATAGATGGCTACCCTGTGCCCAGCTCCTCAAATAACACATGGGGAGGCCAAAAT
TCCCATTCTTTCACACTGACATGGACCTCCCATCCTAAAACAGTAAGAAACTTGCCAGAACATACTCAGTCCTTCCA
GAGTCCAAGACCCCTCATGCTGGAATAGATGCTATTCTCCTCGGATCCTCCTCCTACCTCTACTGCTGCTCCCACTC
CGTTTCAGACTTCTTTTCCTCCCTCCCCTGACCCTTTAAGTGCTGATGTCAGATAAGACTCAGCTCTGCTCCTCTGC
CTGGACTCTGATGGCTCCTCTTCCAATGTCTCTACCACATATCTTCTGCCAGCTTAAAGGCCCTGCTGTACACTGAC
GATTATGTCTCCCCCAAATTCGTGTGTTGAAACCCACCCTCAATGTAATGGTATTAAGGGGTGGGGCATTGGGGTGA
TTAGATCCTGAGGGTGGAACCCTCAGGAATGGGATGGGTGCCCTTAGAAAAGAAGCCCTGGAGAGCTCCCTCTCCCC
TTCCATGGCCTAAGAACACAATGAGAAGACGGGCATGTACAAACTAGAAAGTGGGTTCTCACCAGACACCACATCTG
CTGGTGCCTTGATCTTGGACTTCCCAGCCTCCAGAACGGTACAAAATACATTTTTGTTGTTTATAAGCCACCCCGTC
TATGGTATTCTGTTACAGTAGTCTGAAGGTCTAAGATAGGCTCTCCATGAACTCTATCCAAATGCCCCACAGGTACC
TGAATCCACCTACATCCTTAATCAAGCTCATCACCTCCCCTATTCCTAGACCTGTATCTCCTCCTCCAGTCCCTTTC
CTGGTCAACGGCACCAGCATGCACCAGTCTCTCAGGCCTCCCAGTCATCCCGGACAGCCCCCACCTTCTCACTCCCT
TCCACATCCTTTCAAGTCAGGTTAATCACACCGCCTTACCAATCTTGGCAAATGCTAGTTTCACATCTAGTGCCCCT
ATAGGACTGTAAACTTCTTGAATATAAGTGTATTGATTAATTTCTCCTGTCTGTCTCCTGTGCCTAACACAATGTCT
AGTACCGTGACTCATAGTGAAATATATCCTACGTCACAAACACATGCACATACACATATGGAAGCAAAAATGCCACT
AAACAATACTTATCCTTACTTCATGAGATGCCTTCTGATTTCCTATTTGGTTTCAATTTTTGACCCTTAAGCCAGTT
TCTAAACACATTAATGGATCAAATAATAGTCTGACACACATGGGCTAGCATATCATAGGTGTTTTAATGAACATTGT
TGTATGCTTGCTTAGAGTGTGTGCATGGCCTTGTAAGGTTTTTTAATCATCACTGCCATTTTATTTTATTTTTATTT
TTTTAGGGCCACAGGTGCAGCCTATGGAAGTTCCCAGTCTAGGGGTTGAATCGGAGCTGTAATTGCCAGTCTGCACC
ACAGCCACAGCAACACCAGATCTGAGCCTCGTCTTTGACCTACACCACAGCTTGCAGCAATGCCAGATCCTTAACCC
ACTGAGTGGGGCCGGGGATAGAATGGATACTAGTTGGGTTTGTTTCCACTGAACCACAATGGGAACTCGCGTCATTG
CCATTTTACAGAGGAGTTAACCGAACCTAAGAATTTTCTTTATCTGATTCTAGATTCTGTGGCTTTCCACAGCACCC
CATGGGCTATAGGACCTCTCCTAGCCCCAGTATTTTTTTGCTTTTTAGGGGCTGCACCCGCAGCATATGGAGGTTCC
CAGGCTAGGGGTCAAACTGGAGCTACAGCTGCCGGCCTACCACAGCAACGCCAGATCCGAGCCACGTCTGCAACCTA
CACCACCGGTCATGGCAACGCGGGATCCTTAGCCCACTGAGTGAGGCCAGGGATCCAACGTGAAACCTCACAGTTCC
TAGTTGGACTCATTTCCGCTGTGCCACCACGGGAACTGCTAGCCCCAGTATTTTGTGATTCATCTGTTGCCATTGGC
TAATTGCTGTCAGAATCACTATGTTGTTGCGCAAACATTTGAGTCAAAACATCCAGACTCCCCACCTCCCGGGATGC
CACGCCAGTCACTCACACACACACACACACACACACAAAATCCGGACCCTGTTTTAAGGGTCTAATAGATGCTAAAA
CTCTGTCTCCCCTGTCGGGAATGTTCTCATGGCCCTGTTGCCTACACAGCCCCTGCCACCTCCTGCTGAGCTGTGGA
TTTACTGAAATAGGGCAACGCTTCTTTTCTTACTCAGGATTAAACCAGTCCACTAGCGGAAGCTCTCCTCTGTTGTC
TTCTTTTCTTTGTTCCTTTTCGTTGCCTATAGCGTCTTCTTCTTCGTGGTAACTGTGAGTCCTACGTACAAACGGAA
AACAAGCTGAGGAAGGCAGGGAGGGTGACCCATGTGCCAGAATGAGAGTGAGGATCTTGTGAAAACAGATTCCAAGG
CAGAGAACACGTGCGCCAAGCAAATGTCTACAGAAGGCTTGTGATACTAAACATTTATTCGTAAAGACGTCCGTCTG
ATGAAAAGGTTCAGTGCTCCCCTTTTTCATCATCCTTCCAGACCAGCACAGTTAGCAATGTAATGACCCAGCAATTC
TCAGGTTCTGTCAGGAGCAGGGAAACCTGATAAAACAGTCCTTATCAGCGTATGTAAGCTCATGACAGCCTTTCCTG
CAGCCTCAACTTCAGCCTGAGCCTCACTCACTCCCACATCAAATGGGAAAAAACAAAACCTTGAAAACCAAACTTAA
TGCCCATCCCCACCACGCAACAGAGTCCTTGCATGATTCCAATAAGCCAGAAGGACGAGGCGACTGAGAAGGTCATG
GCTGTGAAACCATTTTATTTGGACTCTACAGCCTTGAGCAGATACACAGATGGCCGTTTCCCAGTCTTACCCATTGT
TAAACCAGCTCGGAAACCACCAGCCCCTCTGAGCACTGCTGCCAACTTCTGGGTTTCTAAGAAATGAAAAAGATGAC
AAACATTTTTTAGAAAATGAGGCAGTCCCAAACTGGGGCAGGGGGTGGGGGGTGTTCCAAACTCTTTTTATGGCAGA
TCACTTAAAATCATTTTTTAAAAAATCACTAATTCGTAAAATGAACAGAAATGAAGCTGCTCCAGCTGAATGACTGA
GGATGGACCCGACACTCCCCAGATCTCCCCTCCCTTGGGTGGCCCCCGGCACTCCGCTGGTCCAGGGAGCCCTCGCA
GGAAGAGAAGGGGAGAAGAAGAATGACAAGGGGGAGGGCACTAATCCATAAATCCAAGTCCTGGATCTGCCCCTTTC
CTGTTGTGTAACCCTGATAGGACATTTTTCCTCTCTGAATCGCCATTGCCTCCTCTGGAAAGTTAGAGAACAATGAC
AGCACCAAACCTACCATGAAGATGGATGGCTTCGAAGACTAAACAAAGTAGCCTACGTAAAAGAGCTTTATAAGCTG
AAAATTACTGTAGTAAGTTGTAGTCTTAAAAAAGAAAAGCCCACATTTCCAAGAATGATCTCTTGCTAAATGAGGAG
AACTGGAGTTGCTACAAAGGTCAGCAGTGACAGATTCAGGAAACCTGAGGGTTTCTAAACCCGAAGCTCAGCAAACT
GTAATCAGAAGCCGTTTTTCTCCACACACATGCTCAGATGTCCACACTCACTGTGAGAGTCTCTCCAAGGCGTGGAC
CGTCTAGAGGAGGGACAAGAGGGGGAAAGCCAGGAGCTGCCATGCCCTTTGGTTGGACAAATGAGGTGGTGAGGCAG
GAATAGGCATAGTAGTAAGAAACTTACTTTATTTTACTTTATTATTTTATTTTTTTTGTTTTTTTAGGGCCGCACCC
GTGGCATATGGAGGTTCCCAGGCTAGGGGTCTAATTGGAGCTGTAGCTGCCGGCCTACGCCACAGCCACAGCAACTT
GGAATCTGAGCCGCCTCTGTGACCTACACCACAGGTCACAGCAGCACCAGATCCTTAACCCACTGAGCAAGGCCAGG
GATCGAAGATGCATCCTCATGGATACTAGTCAGATTTGTTTGCACTGCGCCACAACTGGAAGTCCAAGAAACTTAAA
GTCCATCTACTTTCAGGAAGTGCTTGAAATGGCTTATGAAGAAAGTGTGGTTACGATAAATAGGAAAACAATACAAG
AATCAAAACAAAACAAAACGAAACAGAGAAACATTTTAGTCACTCGGGTGTTTTCACATGACTTTGGTCATCCCAGC
CACTCTGTGAGAACAAAATCTTTAACTTTATTTTTACTTCATAGCTAAGATATTGGCAAAATGAGTTTGAGCAAATT
GCCAAGATCCCATGGCATCTAACAAAAGCCAGGATTTAACACCAGGGGATAAATCATATCAGATGAAGGCTACTATA
AATCAGCTATACTTTAATAAGAAAAAATGTTTTAAAAAAAATGAAGGCCAAGGAAAATGCAAGCATTTAAGCACAAT
ACTTTGCTCTAAGCTTCCTAGCAACCAAGTCGAAGATAGGAAAAAAAATTTAGAAAAATGAAGGCTTAGAGTCCTTA
ATCACCAGTAATAGTAATAATAATAAATAATAATAATACACACACTAGTTTATCAGGACACCCAGCCTTTCTTCCTA
ATCCTTTGTCTTGGCAAAATTTCTGGCAAGGGTCTTTATACCACATGTAGTAGGTAGCATAATGGATAATATCTACT
CTGATTCTTTTTTATGAGCAAGGCAGGAATGTTCTCCAAACAACATCACTTAAAGAGATAGATACTTGATGAGAAGC
AAAGGAAAAACACAACTCATGCTCTAGAAAGGCAAGTCTAGGGGCTGGAGAAGTACAGCTCAGACCCCTGGAACCCC
ATCCCTCTCCTCCACCTAGGACCACAAGTGTGTCACCACCTGCCATGTTAAGAATGGACTGTAGGGCCACCAGGGTC
ACATGGAAGGTGACCTAGAGATATCTGGAATTCAAAGCACTTACTTTGACTGGTATATCCAGAACAAAGAACCTTCT
GGGCTAAAAGCAAATGGAAATAAAAACATATCATGTTACTTGGAATGCAGAGAAAAGCTATTTTGCAATCATTATCA
TTGAAACCCTAGGCTGAGCTGAGAGCCTGGGTTGTGGCTACTCCCAGGTTTCCACCTTCGAGATCGAAAAAATGATA
TCACGGGACTCTCGTCATTTCAGAATTACTCAGATCAAACGGTGGGAGGGAGGTCTCTGGAAAATATCAAATCTTAG
TTTAAAGAAAAAAAAAATAGATGGCAGCTCTTATTGTCCAAGGTGGCTTTGCTGAGGGAGAGAGGCTCCAGAGATGG
GTCCCAGGAAGACCACAGCCCACCCATCCCTCACCCAGGATTTATCTTCCTCCAGAAAAACAGGTCTTGCCTCGCTG
GCTCAAAGCTGTCTACAGAGTAGCCTCAAAGGGCACTTCTAGGAGTTCCTGCTGTGGCATAGTGGGTTAAGAATCTG
ACTGCAGGAGTTCCCATCATGGCTCAGTGGTTAACGAATCCAACTAAGAACCATGAGGTTGCGGGTTCAATCCCTGG
CCTCGCTCAGCGGGTTAAGGATCCAGCGTTGCCGTGAGCTGTGGTGTAGGTCACAGACAAGGCTTGGATCCTGTGTT
GCTGTGGCCGTGGTTTAGGCCGGCGTCTACAGCTCTGATTCGACACCTAGCCTGGGAACCTCCATATGCCGCACCTA
GAAAGGCAAAGCCAAAAAAAAAAAAAAAAAAAAAAAAAAAGAAAAGAAAGAAAGAAAGGCAGAAAAAGAATCTGACT
GCCGTGGCTTGGGTCGCTGTAGATGCACAGGTATGATCCCTGGCCCAGCACAGTGGGTTAAAAGATGTGGTGTTGCC
GCAACTGCAGCTCAGGTTGCACCTGTGGCTTGGATTCAATCCCTGACCCAGGAATTTCCTTCTTTCTTTCTTTCTTT
CTTCCTTCCTTCGTGGAATTTCTATATGCCATGGGTGTGGCCATTAAAAAAAAAAAAAAAAAAGGTACTTCTTAAGC
TAACAAAAGCAGTGAGACCATCCTACAAGACGGGATCAGTAAATATATGACGACTCTAGCAGACCGCCTCCATTCAT
TCAACAAATACCTGCTGAGCATGCGTTACATGTCAAGTGCCAGACATACAGTGTTGACTGAAACAGACACCATGTGT
CTGTGGTGTAGAGAAGCTGGCAGGGAGGGTGGACCCTATTTTGATAAACACATCATTATAGGACTTCAAAACTCCAA
GAAAGCATAGGAGCACTTAACAGGAAGACCTCGAAGGCTCCCCAGGGGAGGGGATGATGTTTTAGCTGAGTTCTGAA
GGATACATAGGAGGCCCAGTGAAGAGGGATTAGCAAGAGTGTGCCTAACAGAGAGAAAAACATGCAAAGGCCCCAAG
AAAGGAAGGTCGCATATTTATTTATTTATTCATTTATCTTTTGGGGTTGCACCTGCGGCATGTGGAAGTTCCCAGGC
TAGGGGTTGAATTGGAGCTACAGCTGCTAGCCTACACCACAGCCACAGCAATGCCAGATCTGAGCTGTGTCTGTGAC
CTACACCACAACTCACGGCAATGCCGGATCCTTAACTCACTGAGTGAGTCCAGGGATGGAACCTGCATCCTCATGGA
TACTAGTCAGATTCGTTTCCACTGCGCCACATCGGAAACGCCTGCCCTCATCTCTTAAAACAGAAACAAAAAACCAC
TAACCACTAATATTTGTTTGAGATTCTGCCAAAGCCCCGATCTCCTCCCTCTGCCTTCTGCCCCAGCTGGGAGTCCA
CATCTCCTGGTAGGAATGAAATACATGCCTTCCTACCACCTATGGTTTCCCCTCTAAGCTCAGTACCCATGGACCCA
GCTCTAAAGTCCCTTGTTTCTAAATCTGTCTATTGATCTGATAATATTCATAATAGCTAATAGTTGGCTGGGGACCT
TTCTAAGCAACTGACATGTATTAGCTCATTAAATTCTAATAACAGTCAATGAAGGAGGTTCTATTCCTCCTCAGAGG
GACAGAGGCAATAAATTATTTTGCCCAAGGTCATACTGCTAAGGGAAGAAACAGTATTTGAACCTGGGGAATCTGAC
TTCAGATCCTACAAGAGGGGGAAGGGAAAGGGGCAAGAGGAGGGGGAGGGCCCGTGCCACCCAGCACTCAGGAGCCC
CACCCTCCTGCCGAGGCACTCAGGGCATCAATTTATAGATTTGGATTTGCCACCTCGTCCCATCTTTTTAGTAACCC
CTCCCTCTTCCTCATCTCACCCTCCTTTCCCAGAAGCCTTCAACACCTCAGGTCACAGCAACAACCACCCTGAAGTG
TACGGCATTTAACACATATTCATCCTTCAAGGCACAGCTCGGATGCCATCTCTTCTGAGCCTTCTTTGGTATGAACC
TAGCACAATGCCTGGCATACAGTAGGTGCTCAATAAATATTTCTAAATGAGGGAGTTCCCGTCGTGGCGCAGTGCTT
AACGAATCTGACTAGGAACCATGAGGTTGCAGGTTCGGTCCCTGCCCTTGCTCAGTGGGTTAACGATCTGGCGTTGC
CGTGAGCTGTGGTGAAGGTTGCAGACGTGGCTCAGATCCTGCGTTGCTGTGGCTCTGGCATAGGCTGGTGGCTGCGG
CTCCAATTAGACCCCTAGCCTGGGAACCTCCATATGCCTCGGGAGCAGCCCAAGAAGTAGCAAAAAGACCCCCCCCC
AAAAAAATAAATGCAAAACATAGATCCATCTCCAAGCCAAACATAATCTTGCCCTCCCTGAACTCTCACGTTCCTTT
GCTCTCTCTCTCTGACATCCTCCTTCTAGCCTGTGTTGTTGGGCTTTCATGGGTACCTCTGCCTGCTCCATCTACAG
CATAACCCCTTGAGGGTAGGGATTCTCCTTGGCGCACACTGTACCCCTCGCAGCATTTGGCATGAACAACCAGCTCC
AGAAGGAGCCCCAGATGATGAATCAGAAGATCTGAGTTCTAATTAGAAGTTAGACATAAGTTCACTGTTAAGGCATT
TCACCTACTTGTCCATCGCCTGAACAATGGAAACCTTGACTAAAGGAAGGGTTACCCAGGTTACCCAAGTCAGACAG
CCCTGGACCTAAATCTTCCTAAAAATGTGACCTTGAACGTTCACATTTAATATTGTGGAAACTCAGTATTCCTCATC
TAGAAATGTGGACTAACACTGACCTTCCAGGGCTGTTTTAAAAACAGGAGGGAATGAACAGTGGAGTTCCTGGCACA
AGCAAACACTCAATAACTAGTAGCCGCTAACATCAAAATCACCATCACCATCATTACTTTATTATAGCTCTTAAAGT
TTCTTCCACCTCTAAAATTCTAAGCTTGTGGCTCAGTGGCTTAAGAACCCAACTAGCATCCATGAGAATGTGGGTTC
AATTCCTGGCCTCACTCAGTGGATTAAGGATCCAGTGTTTGCCATGAGCTGTGGTGTAGGTCACAGACGGGGCTTGG
ATCTGGCGTGGCTATGGCTGTGGTGTAGGCAGCTCTGATTCCACCCCTAGCCCAGGCATTTCCATAGGCCACAGGTC
TGGCCCTAAAAAGAAAAAATAAATAAATAAAATTCTAAGATTTTTTTTTTTTTTTCATCTAGCCTTTAACCAAATGC
TGTCCTGGATGACATTCTTAAACAGCTGTATGTGTTTGATGGAGTTATTTTGTAAATCTCTTTTTTTTTTTTTTTCA
AGGGCCTTACCTACAGCACATGGAAGTTCCCAGGCTAGGGGTCAAATCAGAGCTGAAGCTGCCAGCCTACACCACAG
CCACAGCAACACCGGATACCTGACCCACTGAGCGAGGCCAGGGATCGAACCTGAATCCTCATGGATACTAGTTGGAT
TTGTTACCACTAAGCCACAACAGGAACTCCTGTAATCCTCTTTAGCTACAGTGCTACCCACCTGTCTAAGGTTAGTG
CCCTCAGCTCACCTCAGACCAATTCACAAGGTGGCAAAGAATCTCCTGCCTTTTAAACCCCTTGCAGATGTTCAAAT
AGATTCCTCACATTGAAGAATGATGTGGCTGCAGTCTGGGTGCCAGACTACGGCCCTGAAGAGCAGCCAGAATCTGC
TCCAGTTACTGTGAAGAGAGAGTGTGCCCAGCACTGCAAAACAACCCTCTTTATGGGAGGCCAGCACCAATATGCAC
TTCTGGGCCTTTGGCTTCTGTGTTTTAATTTTGTGAAGTACCCAAAATATGGAAGTATAACTCTGGCTGCAATTCAA
AACAATCAAGAGTTCAGAGCTTGAAGGTTGCCTACACAAGCATCTCAACTCAGGTCAGGAACCCCATGGGGAACTTG
CTCTTCTGTTAGATTCTTTCAGCCCCTAGAATTTTTTCTTTTTCTTTTTCTTTTTTCTTTGTAGGGCCAAACCTGTG
GCATACGGAAATTCCCAGGCTAGGGGTAGAATCCGAGCTACAGCTGCCAGCTTACACCACAGCCATAGCAACTCCAG
ATCCTAGCCATGTCTGCAATCTACACCACAGCTCATGGCAACACTGGATCCTTAACCCACTGAGCGAGGCGCGGGAT
TGAACCCGAAATCTCCTAGTTCCTAGTTGGATTCATTTCCCCTGCACCACAACGGGAACTCCTAGAACTCTTCCTTC
TATTTGCCAAAATCTCCTGTCCTATGCTGCCCTCCGGACAGATGGTGATAGTGGTGGTGGTGATGGCAGCCAGCGCT
TACTAAGTACGTTGCCCTTAGTGCTTTATTCACAACTTATTTTATCCAACAACCCTATGAAGCAGGTACTACTATCA
TCCCCATTTTTAAAGATAGGGAAACTTGCCCAAAGTCACAGAGGAGGGAAGTGGTGGCACAGGACCAACCCCAGGCA
GCCTAGCTCCAGCCTCCACTGAGAATATCTCCTCAGTCCTCAAGTACCTAAGGGAGCCCCAGGGTCTCTGCATCCAA
CGCTGTCATCTTTTCTTCAGAGGAAGTACCACAGTTTCCTCAATTCGAAAAGGTTGGTTTGTAGACATTTGTTCACT
CTCTAGCTCGTCTTGTTTTTCTTAAAATGAGTTCTTCAGAATGAGAGGGAATAACTGTTCCAGAAGTGGTTAGATCT
ATGAAGCATCCAAAGGAATGACAGCTTCTTATTCTAGGGAATCCACCTCCTCCTTTTTTTTTTTTTTTTTTTTTTTT
TTTGGCTGCACCTGCAGCATGCAGAAATTCCTGGGCCAGGGATCAAAGCCAAGCCATAGCAGTCACCTGAGCTGCTG
TAGGGACAAGACTGAATTCTTGAACCCGCTGAGCTAAGAGAGAACTCCCTAGAGAATCCTCCTTCTACTGATGGACC
TGAAGATGCAGTTCCTTTCTAAGTGGCCAAAATGGTCCTGCTGGCTCATCAAGTCTTAGAATTTAAGAGACATTCTA
ACGTTAATCCAGGCCATCATCCTGAACTTGAGGGGCTACTAAAACACTACCCATCAAAATATCAATGGTGATGACAT
AGCTCTCCAGGCCAAGTTGTTTTTTGGTTTTTTGTTTGTTTGTTGTCTTTTTTCCTTTTAGGGCCACACCTGTGGCA
TATGGAGGTTCCCAGACTAGGGGTCCAAGTGGAGCTGTAGCTGCCGGCCTACACCAAAGCCACAGCAACACCAGATC
CAAGCTGCGTCTGCAATCTACACCACAGCTTACTTCAACACCCGATCCTTAAGCCACTGAGCAAGGCCAGGGATTGA
ACCCACAACCTCGGGGTTCCTAGTCAGATTCATTTTCCGCTGCACCACCACGGGAATGCCTTCAGGCCAAGTTGTAA
GGTGGCCTTTTTGAAAGAAAGTCCAAGCGGTATCAATACCTCTTAAGTCAAAGCCATCATGCATTTTGGTAGCTGCT
TGCAGACATTTCTTTCTGTCAGAAGCGTCTCCAGCTGGAATCTCCAAGGCATCGTAGTTTCCAAAAGCAAAGAAGCA
GCGTCAAATATTTGGGGTGAATCCACTGATGAATTTGAAAACTCAGAAATGTTTAATTCATTTTGCTTTCCAGAGTT
AAAAAAAAAAGACAAAACACCCAAAAGTTTAGCCAGGCACAAATGAATCACCAGCGACTCAGTGTGTTTTGCAGCAA
AAGTCAACAACTTGAGTTGTTCCTTTAAACTCTGCAAATATTTTAGGATTGCAAAAATCAGGGTGTATTTCTCATGG
AATTCCTGTCTGAAAGTTCTCAAGGTAACTTCCATATCTGGTCATATAAATAATTTAATATTATATCTTGGTCTTAA
CATGACCTTATTATTTCTGGCTCTAGCCTACCCAGAACTGCAGAGGTATAAAAATCAGGACAATGGCAACATGGCAG
GAAGGAAGATAATTAATTAGCTGGAAGGTACTTGAAGATCTAATGACTTTAAAGACGGTATTTAAGGGCTCAGGGAT
ACAGGAAGGGTAGAATATTTTCTTTCTTTCTTTGCTTTTTAGGGCCGCAAGTGTGGGATATGGAAGTTCCCAGGCTA
GGGGTCAAACTGGAGCTGAAGCCACCAGCCTACGCCACAGCCACAGCAATGCCAGATCCGAGCTGCATCTGCAACCT
ACACCACAGGTCACGGCAATGCCGGATCCTTAAGCCAAAGAGCAAGGCCAGGGATCAAACCCACCTCCTCTTGGATC
CTAATTGGGTTTGCTGCCCCTGAGCCACAACGGCAACTCTCTGGAATGCTTTCTTTACGGTGTCAGTGAATCCTACT
TTTAATGCAAGCTGGTGACTTGGCTGATAACTAGGAGATTAGAGGAGACTTTCATCAACATCATTTCATCATGTTTC
ATAATTACCTGTTGATGTATTCCCAAAACACAACCATTACAGTTGAGACAAGCAGCATTGACAGAACCACTCTTCCT
TTGACATTCATTATTTTCTCCTGGGAAAAGAAAAGGAGAAGGGAAAATTAGATTAAATACACCCAGAGTGGAATATG
GTTTTTTAAGAAGTGCTTATACCAATATCTTTTCTAAAAGGAAAAGTTGATGAATAGTCAACGAGCGCTAAGGAGTG
CGTTCTACCTTAATTTGCATAGGCCTACACTGGCAAATTAGCCAAGTCAATGAACTGACAGGGCCGTCTGGGTTGGG
AAGGATACTAAGGCCATTTTGAGGCTCAAAGGGGAAGCATCCTGACTGATCCCAAGGTCCACCGAGATGTGGGAGAG
TGACGGGTTTAGTTAATGGTCCCTAAGGGCTCCAGCCGCCCCCAACTCAGATGCCCCACCTCGCATCACAGACTAGA
GGAAGCATCCGTTTCCTAGGTCTACTGTCCCTGATATACTGACTATGTACCTTATCCTCAAAGAAAAATATACCCTG
GTCCTTTATTTAATTTCATTTAAATTTTAGGGCCACACTCACAGCATATAGAGATTCCCAGGCTAGGGGTCGAATCA
GAGCTGTAGCCACTAGCCTATGCCACAGCCACAGCCACACTAAGTCCACGCCTTGTCTGCGAACTACACCACAACTC
ACGGACAGCAACGCCAGATCCTTAACCCACTGATTGAGGCCAGGGATCAAACCTTCGTCCTCATGGATGCTAGTCAG
ATTCATTTCAGCTGAGCCACAATGGGAACTCTCACCCTGGTCCTTTATAATCTAGGCTCTGCCACTTCCCACCCAGC
TTTTCCCCAATGCACCCACACAAGTGGCAAACAGTCGGTACATTCGTATTTCTTGATCGCTGCATGAAATTGTAGTT
GAAGAGGGAAGGGATGCTGGGTGGAATAACAGGTTGCGGAGTACTTTAATTTGGGTGGAGATAGAAAGATATTTATT
TCAAATGGAAAGGACAAGAAAAGTGTGGCAGCTAGCCACATATCAGCAATACTCATAAACAAAGAATGTAACAAAAG
ATAAAGTAGGGCATTACATAATAACAAAGGGATCAATACCAGAGGAAGACATAACATTGGTTAACATATATGCACAC
GATATCAGAGCACCTACATCTAGAACGCAAATATTAACAGACATAAAAGGAAAACTTGCACAATTACATAATACTAG
TAGAGGACTGATTCGCAACATTTTGTGGGTCTTGTGATTTTTTTCTTTTTAGGTCTATTTGTCTTTTTAGGGCCGCT
CCCGCGGCATATGGAGGTTCCCAGGCTAGGGGTCGAATCGGAGCTGTAGCCACCGGCCTACACCAGAGCCACAGCAA
CGCGGGATCCAAGCCTCATTGGCTACCTACACCACAGCTCACGGCAACACCGGATCCTTAACCCACTGAGCAAGGGC
AGGAATTGAACCTGCAACCTCATGGTTCCTAGTCGGATTCGTTTCCACTGTGCCGTGACGGGAACGCCAACATTTTG
TGTTTTAGATGTCATAGTTTACATCTTCACAGCTATCCTTCAACTATATAATTTAGTCTTTTAACATCTGTACTAGT
TTATTTAAGTGTTTGATGCAACACCTTCACTATATATTTGACTTTTCTAGTCTTATTATTTCCTTTCTGTATTTTCT
CATATCTTGTTACAGTTTTTTCTTTTTCATTTAATGAAGACACAAACATTTCTTGCAAGTCAGTGTAGTAGTTGGAA
ACTCAGTTTTTCCTTCTGGGAAACTCTTTAGTCACCCTTCAATTTGGGGAGATGACTTTAGAGCTTCCCAAGGGATG
AAGATAGGATGGGAAAGGATGACAAGGGCCGTGAGAAGGGATGAGAATATTTTGGAAACAGCATCTATACCAGGCAG
ACAAGAGAAAGAGCTGCTCGTGTTTGAAAAAAACAAAAGCAAAAAACCTGGACAAGAAAAAAATAGTGACTGACACT
GTCCCCCTTGAGTGGCTGGTGCTAGGCAGTCAGAAGGGGGGCAGAGGCAGTCAGAACCTGGAAAGGTATGGAAAGTA
GGGTGGGGAATCCCAAAAAGCATCTAAAGCTGGAGAATCCCCTGATCCAACTTCACCTAGAGAGACCCATCTGGGTG
CTGAGTGTGGAGAATGGAGAAAAGGACAAGGGCAGACCGTTCTCATGACCATAAAGAGGAGGTGGCCTGGCTCAAAG
GGTGGCTTGATTCAAAATATACTTTGGGAGTTCCCGTCGTGGCGCAGTGGTTAACGAATCCGACTAGGAACCATGAG
GTTGCGGGTTCGGTCCCTGCCCTTGCTCAGTGGGTTAAGGATCCAGCGTTGCCGTGAGCTGTGGTGTAGGTTGCAGA
CGCGGCTCGGATCCCGCGTTGCTGTGGCTCTGGCGTAGGCCGGTGGCTACAGCTCCGATTCAACTCCTAGCCTGGGA
ACCTCCATATGCCGCGGGAGCGGCCCAAGTAATAGCAACAACAACAACAACAACAACAACAAAAAAAAAAAAGACAA
AAGACAAAAAGACAAAGAAAAATAAAATATATACTTTGACAAATACCATATGATATCACTTATAACTGGAATCTAAT
ATCCAGCACAAATGACCATCTCCACAGAAAAGAAAATCATGGACTTGGAGAATAGACTTGTGGCTGCCCGACAGGAG
AGGGAGGGAGTGGGAGGGATCGGGAGCTTGGGGTTATCAGATACAACTTAGATTTACAAGGAGATCCTGCTGAGTAG
CATTGAGAACTATGTCTAGATACTCATATTGCAACGGAACAAAGGGTGGGGGGAAAATATACATGTAAGAATAACTT
GATCCCCATGCTGTACAGCGGGAAAAAATTAAAAAAAAATATATATATATATACTTTGGAGAGAGAATTGATAGGAC
GTGGTTGGTAATTTTGTTATCAGAGATGAGACAAGGAAGACCCAAGATTTCTGCTTAAGCAGGGGGGTTGTAGTATT
TTCTCAGATGGGCTGGAGGAGGAACAGGCTTGGAGGATAATAATCATGAATTCCCTTTTGGACGTGTGAATGTCGGG
GAGTGTGCGAATACCTAAAAGGGGACAGGGAGACAAGTGGACATTCAAGTCTAAAGTTCATCAGAGAGATGTAGGCA
GACCATGCAATCGGAGAAGTTGTTCATGGACCAAGGAACGTATCGGATCTGACGTGAAGGGAACGAATTTGATTACC
CAGGAGAGAATGCAGAGAGAGAAAGAGGAAGAGGAGGATGCTGGGCTGAAGCTTTAGAGGTAGGATAGAGGAGGGCC
CAGAAGGAGAGGACCAGAAGGTAGCAGAGACAGAAGAGTGGACACCTGGGAGCCAATGTCACTGCCTTTGTGAAGCC
ACTTCCCACCCCCACCCTGACCACGGCTGAAGCCCTTTTCTCTCCTCCGGCCCCCATCCCTCTATTCCTTTGCTGTA
CACATCGCCCTGGGAGTCGGCTCACCGGATAAGACCTGCATTTTGCTCTGCCTCCTCTACCTGCTTGTTTGAGCTTC
CTGAGGGCAGGAGGGATGACTTCTTCGTCACCCCTGAATTCCCAGTGCCCCACAGAGAGCAGAGAAGGCCGTCAATA
AATAATGAGTGGTTTGAGCTTCCTGAGGGCAGGAGGGATGACTTCTTGATCACCCCTGAATTCCCAGTGCCCCACAG
AGAGCAGAGAAGGCCGTCAATAAATAATGTGTGGGAGTTCCCGTTGTGGCTCTGTGGTTAACGAATCTGACTAGGAA
ACATGAGGTTGTGGGTTCCATCCCTGGCCTTGCTCAGTGGCTTAAGGATCCGGCGTTGCCGTGAGCTGTGGTGTAGG
TTGCAGACGCGGCTCAGATCCCGTGTGGCTCTGGCTCTGGCGTAGGCCTGCAGCTACGGCTCCAATTAGACCCTTAG
CCTGGGAACCTCCATATGCCGCAGGACTGGCCCAAGAAATGGCAAAAAGACAAAAAAAAAAAAAAAAAAAAAAAAAT
GACGTGTGAATGAAATGAGAATGGCACTGAGATGTGTCCTTTCAGGGGACGGGTTATTCTCCAAATATTTGCAGAGA
GGGTTCTGAGGTGACTCCAGGCTTAGATCTCAGGTGCTCCATCACCTCTGTTGTGAAATCCAGTTAAAGAAGAGAAA
GTATGGGATTATCAGCCATGTCACTCTATTCCTTCTTGCTTGGAAAGTGAGCTCTGTTTGGAAACCTCTGATTCAAT
CGCCACCTTTCGGATACAATCATGATAGGTGGTGTTCCAGAGACGGTGAGAAGATGGGGAGATGGAGCTTCTTTCCT
GTGAGCACCTCAGGTCCTGGCACAAACAGCCCGGGGCCCAGGGCAAAGTTACGAAATGCACGGGGCTACATGCAGCT
CGGCCCAGATGCTGGAAAAAGCCACTTGACTCCTACACCAACAGCATTAGCACTGAGTGCGAGGAAAGGCCTGGGTT
TGGGAGCAGACAGATCGGGGTGGAGACTGTGGCCACTGTGGCCATGCCTCTCTGCCGTTGTCTTCACTCCCAGAGAA
GTGTGGGTGGTGAGAGAGCTTGGGAAGGAGGTGGGGTCTGGAGACACCCACAGACTGGGTAACCCTGAACATGGAGC
AGTTTCTCAGACCCTCATCCAACTCCAAGCTCTGAAAACCAAAAGCCTGTTTATAATTCAGTTGGCATCCAGGCCCT
GACACGAGGCTATTTATAATCTTTATCACTTAGTGAGACTGTTTAAACATTTCTTTGCATAAATATTGATGTACATT
GTTATGTGCTGTTGCTGCACTGGAGGCGTTACATAATATAGGATAAATATTCTGCATTTGAAAAATTCTAAATTCCA
ACATATCTGGCCTTAGGCATTCAGGAAAGGGATGGTGGACCTCTAATTGATCACATTAGATGGGTCTCCTCATCTTT
AAAATGGGAATTAAAATGGTGATGACTGCAAGAGATGGTGTCCATAAAATATTTAGCATCATGCCCAGCATCATATA
AAAGCTCAAAAACTGCTAGTTTGTATTACTGGTATCCATAAAACAGGCTGTTGGGAGGATCCAGTGAAGACAGCACA
GCGCCTGGTACTTAGCAAGAGCTCAAAACGTATCGGAGGGAAAGGAATAAGCATTTTGGAATAAGAATGTGTTAAAC
AATAAAGTACAAATTGATGCAAATTAGGGCCTCTAAAGGTTTATCCATCTGTTCTATGCTGCAGACTGACTAAAAGC
TCCTGGGAAATGCCACGCAACTTTGATTTTCTTTGATCAAGCCCAGGCCATCCAAAGCCTTGTCATCCCCACCTGCT
GAGGATCAAACCCTGTGTAAGAAATGCGAAAGAGAGAAACACAAACTCCTGGCAGAGAACGGATCAGGGAGAAGCTG
GTATAAAATCAGACACACCTCCTAATCCTTTCTCCAAAGGCAAGTGTTTTTCTGTTTGTTTTGGTTTCAGGGTTTGT
TTGGGTTTTTTTGTTTTTTGGTTTCTTTTGGTCTTTTTAAGGCCACACTGGGAGTTCCCCTCCTAGCTCAGAGGTTA
ACAAACCTGACTTGTATCTGTGACCATTCAGGTTCGATCCCTGGACCCGCTCAATGGGTTAAGGATCCAGTGTTGCC
ATGGCTGTGGTGTAGGTCGCAGATGCGGCTTGGATCCAGCATTGCTGTTGCTGTGGCGTAGGCTGGTAACTACAGCT
CTGATTCAACCCCTAGCCTGGGAACCTCCATATGCCAAGCATGTGGCACTTAAAAGATTAAAAAAAAAAAAAATTAA
GGCCACACCCAAGGCATATGGAAGTTCCCAGGTTAGAGGTCAAACTGGAGCTATAGCTTCTGGCCTATGCCACAGCC
ACAGCAACGCCAGATTCAAGCTGAGTCTGTGACCTCCACCACAACTCATCACAACATCAGATCCTTAATCCGCTGAG
TAGGGCCAGGGATTGAACCCTTGTCCTCACGGATACTAGTAGGGCTCATTACCACTGAGCCACAATGGGAACTCCTT
TGTTTCATTTGTTTTTGATTTTTTTTTTTTTTTTTTTTTGGTCTTTTCTAGGGCCGCATCCACGGCTTATGGAGGTT
CCCAGGCAACGCCGCATCCTTAACTCACTGAACGAGGCCAGGGATCAAACCCGCCACATCACGGTTCCTAGTCGGAT
TCGTTAACCACTGAGCCATGACAGGAACTCCTGTTTTTTTAATTTCAGAAATTAGCATCAGAGACAACTCTTGAAGC
CCCCCCCCCCTTTTCTTTTCCTCTGGACCGTAAACATGGCTTGAATCTGCTTACTTTTCGCTGTGGCCAGGCATCAC
TCTTAGAGACTTACAGTTGGAAGCCACCCAAATGAGCCAATATTGCCTCCTTTTGAAAAGCACTGGGAAGGGGTATA
TGCAAGCTTTCTGGAATCTGGAACCCTAGTGTCTCAGGAAAGAAGGGTTGCCAGAATGGCCAAAGGGTTTTTAAAAC
ATTTTTTTTTTTTCTCTGGATTAAAATGAGGCATTTGGCAGCCCATGTGGTCTAAAGCCCTTCACGGATGTGTTTGT
CACAGAATTTTCTAACTCTCTAATTCTCAAGATTGGTGGTTGACTATCTTACCCACCAAATAGGAAAAGTGGGGGTT
GCTTCTACATTTCTCATGGAAGAGGGAGAGCACAGGATTAGAGCCTAGAGAGCACTAGCACCCTGTCTTATAAGGGA
GAGTGTAACCACCTCAGCACCACCTGGGCCCCAGCCCTCAGAGGATCAGGTGAACCCAGCGGGCCCAGTTCCACCTG
AGCCCTCCCACCATCCCACAGGCCCTCCTGCCAAGGCGTTTGCCATTTCTCTCTGCTCCTGGGCCACTCCCACAACT
CAGCCCCTGCAGCGGTTTCCAAAAGAAACCACTTGCACCCCCACTCCCGGGCCTCGTGCAGACTGTGCTAAAACCCA
GTGCATTTCCCAAGGCAGGGCCACGCTGGAAAGCCTGTCATTTCTCCACCTTCCTCCTCCTCCTCCTCCTCCTCTTC
GGCTTCTCCATCCCTGGGGTATCAGACTCTTCCCCAAGGCCCATAAATTAATCCTTCCTGACCCACCCCTAACTTGT
CCCACACAGAACGGTACACACACCCCCTCCACTTCAGAGAAGCTCATGGTTTCACCGCAACTGGTCCAAGTCAAGGT
TTTCCTTCCAGACAGAGTTCCACTCTGAAAGGAATTCTAGTGGCCCTGTTTTTCTCCACCTCGTGTCAGGGGGAAAG
GTGAGCACCTCAGCTGAATCACAGAGCTCTCAGAAGCCCTGGAAAAGCCATTATCTTGAGAGAGCAGCGAGCAAGCA
GTGACAGAGGAAACCAAAGCTTCCAGCAGACTAAAGAATCTTCCTCTCTGCCTGTGACTCTTGCCCTGCCCCTGGAA
CCCATCCTGCCCTGCTAGCTCCACAGGACCCTGGCAAGGGTCAAGAAAGTCAGGTAGTGATAAGTGCAGCAAATGAA
ACACAGTGCGGGGGAGGGAGCCAAGGTGGGGAAGCCGCAGGAACTGACTGGGTGTTACTCACCCTGGACAAAAACCT
CCTATTTTTAGGCCTAACATTTAGATCCAGCATTCCAGGCAGAAATTAGGCCGGTGCTGGGACTGGAATCTGCAGCC
CTACATGCACTTGCCCTGGGCAAGTCCTCTGGCTCTGAGCCTCTACTTACACAGACCAAACGGAGCTTCAAACACCC
TCCTCCAGGGCTCTTGAAAGGACAAAAGGAGACCCCGTCTATGAAGCATGTTGTGCCTGATGCTCAGTAAATGCTCC
ACAAATGCAGCCAGAACAAGGGCGATGCTTTTTACGGGGAGAGATTCAGAAATGTGTGGCTCTGACGGCCGAGCTGT
GGCTCTGTCTGAGAGGAGTCTGGGCCCTCCAGGGCAGCACCACACAGAAGGGTCCAGGGCGAGCCCCCCACGCTGTT
GTGACTGTTGTTGGGGCCAGCTCAGGGTCCCCAAGCGCATCTCGTTTGCCTCTATCGCCTGGCGCGCATGTTGGGCA
GGGAAGGAAAGTCAGGCTCCAGGGTCACCCCAGCACCCACACAGAGCGGGTTTGTGAACCACACGCAGCTTTCTCTG
GCCTCAGTCTCCCCGTCCTTTGAAACATGTCCTGTGGGCTTAACTTCCCTGAATGAGCCAAGACCTGTATGAGAAGG
CAGCCACAGAGCTGGAAGGCTCCTTTTATGAGGACAGGTTCACTGGAGCTCAACTTGCTGCAGTGGCCACAGATTCC
TAGAAGTGGTGATCAAAAGATAGGATTGCCAGAGTTTCCGTCATGACGCAACGGAAATGAATCTGACTAGGAACCAT
GAGGTTGCGGGTTCGATCCCTGGCCTCGCTCAGTGGGTTAAGGATCCGGCATTGCCATGAGCTGTGGTGTAGGTCAC
AGACGCGGCTTGGATCCTGTGTTGCTGTGGCTGTGGTGTAGGCTGGCAGCTGTAGCTCCGATTTGACCCCTAGCCAG
GAAACTTCTATATGCAGCGGGTACGGCCCTAAAAAGCAAAAAATAAAAAAATAAAAATAAAAAAAGAGATAGGATTG
CCCACAAAATGTGTTGAGCCCTCAGGCCACTTCACCCAGAAGCCTCCGGGTCAGGCCCCCAGGCAGGCCTGGGGTGT
GGAGTGGGCAAGGCCCAAATGCTTCCTCCAGGTGAGGTGCTGCCCCTGCCTGGGGGAATCGTTCCAGCCTGGGTGCC
TGTCCTGGGGCTGCAGGTGGAGCCCAGGTACTGACCCTGCTCCCCGCACCTACCTGGGTCCTAGGAGCAACCTGCCC
CATCCAGGTAGACCTTGCTGAGCTCCTTGGAGCCTCTCACTTTGATCCCAAGGAGAAGGAGCTGAACATGATGCTAC
TTGGCTCCCTGCTCACAGGTCACGATCCAGACCTCACAATCACCTGGTGGTGCACCCCCCACTCCAGCCAGGATCAA
AGAGCTGAATTCTCCAGGACTCTGGCTGGACCCACCTGAGCAAGAAACTGCCAAAAGATGGGGCGTTTGAAGGACCT
GGAGCACCTACACACCCCAAGCTTTCCTCATGGTTTCAGTTACAAGATCTGTGTTTGGAGACCTCCCCTTGGGGGCA
GGGACCATGGAAAAGTTCCAGCTGCAAGCAGACCAGCTGGGAGTGGAAATCATCTCCTCGGGCTGCACCATCACGGC
CCTGGAGGTCAAAGACAGGCAAGGCAGAGCCTCAGATGTGGTGCTTGGCTTTGCTGAATTGGAAGGGTACCTCCAAA
AGCATCCCTACTTTGGAGCAGTGGTTGGCAGGGTGGCAAAGCAAATTGCCAAAGGAACATCACGTTGGATGGGAAGG
AGTATAAGCTGGCCAACAGCCTGCACAGAGGAGTCAGAGGATTTGATAAGGTCCTCTGGACCCCTTGGGTGCTCTCA
AATGGCATCAAGTTCTCGAGGGTCAGTCCAGATGGTGAGTTAAAAGTCTGGGTGACATACACGCTAGATGGCAGGGA
GCTCATGGTCAACTCTCAAGCACAGGCCAGTCGGACCGCCCCAGTCAATCTGACCAGCCATTCTTATTTCAACCTCG
TGGGCCAGGGTTCCCCGAATATATATGACCATGAAGTCACTATAGAAGCTGATGCTTTTTTGCCTGCAGATGAAAAC
CTAATCCCTACAGGAGAAGTTGCTCCAATGCAAGGAGCTGCATTTGATCTGAGGAAACCAGCAGAGCTTGGAAAACA
CCTGCAGGAGTTCCACATCAATGGCTTTGACCACACGTTCCGTCTGAAGGGATCTAAAGAAAAGCAATTTCGTGTAC
GGGTCCATCATGCTGGAAGCGGGAGGGTACTGGAAGTGTATACCACCCAGCCTGGGATCCAGTTTTACACGGGCAAC
TTCCTGGGTGGCACGCTGAAAGGCCAGACTGGAGCAGTCTGTCCCAAGCACTCTGGTTTCTGCCTCGAGACCCAGAA
CTGGCCCGATACAGTCAATCAGCCCCACTTCCCGTCTGTGAGTTCAAACACACCCCTTGGTTCTAGTTTTCTGTGGC
CTAAGGAAATGTAAAGATATGACCTGTTCCAGGGTCAGGCTGGAAGCCCCTTCAGGAACCTGTCTCCTACGCAGAGA
TAAGATGAAGATTTAGAGGTTTTAAAAGTGATCCTGTGTATTACTCAGCCATTAAAAGGAAAGAAAGAACGGCATTT
TTAGCAACAGGGATGGACCTAGAAATTATCATGCTAAGTGAAGTCAGTCAGACAATGAGACACCAACATCAAATGCT
ATCACTTACATGTGGAATCTGAAAAAAGGACACAATGAACTTCTTTGCAGAACAGATACTGACTCAGAGACTTTGAA
AAACGTATGCTTTCCAAATGAGACAGGTTGAGGGGTGGGGGGATGCACTGGGGTTTTGGGATGATCATGCTATAAAA
TTGCATTGGGATGACTGTTGTACATCTATAAATGTAGTAAAACTCATTAAGTAATAAAGAAAAGAATGTAAAAAAAT
TAAGAAACAGAAAAAAAAGTGATCCTGTGAATTAAAATTACACAAATGGTAGTTGTCATGATAATCTGAATATTGAT
TTCTTTCACAATGACTGGCTCCAGGCCAAGTCTAATGGTCAGCTCTATTCTCTGTGTAGTGAAAAAGACCCAACCAT
CAATGTCATCTTCTAAGCCCTGACCCTAATCCAGAAGTGGTACCCAGATCCTTGTGTTGGCTCTGTCTCTCCACTCT
GCTTCTTTTCACTCCTTCTTTCTTTGATCCTACTCATTCCTTTTTCCCTTCCTCTTCTACCTCATACCACCTTGATC
TGTGCAGCACTTTGGAGTTTTCAGAGGTCACTGAGCTCATTCAACCTGGTGGTAGAGGGACCTCTCTGCCTCAGTAA
AAGAATAGATGATGAAGTGAGCCACCTGAGAATTAGGGGAGGTAAATGACCCACCTAAAGGCGCACAGCCAGGAAAA
ATTTAGCCTGGATTCAAGATCAGGTCATGCAAATTCAAGTCCTTCTTTGCCTCCACTTCAGTCTTCCAGAGCATTCC
TGGAGTCATTAATGGGAAAAGGGGGGGTCTGACCCTTACTCTGTTAAAGCCAGACCTTCTTTCCAGATATCACTTTT
ATAAGAAGCCCTAGTCAGAGTTTAAATGTATCTCTGAGCCTTATAAATAGTGTGACTTAAAATACAAGATCTAAATA
TCCAGAAAAAAAAAATCTGTGAATTTGATTCTCCGCCTTTGGGGTTACTAAGAAAGCCCAGCCTAGCCAAGACATGG
GAAGGAAGCCGCTGGAGACAAGAGCTGTGTGAGTTCGAGGAGAGGGCCTTGCTGGGACTGCACGCTGCACCGAGAGC
AGACTGTATTTGGTATACGAGGCGGAGTTCCCTCCTCTCCTAAACAATTGAATCACGAGTGATGGGTTTGTGTTGAT
GGTTTTTAAAGAAATGTTATCTTATACTCCTCTACACTAATAATCAGTTGAAATAAAACCAAAATGTGCACCCTCAG
AAAAAAAAAAAAAGAATAAAAAGAAACTGCCAAAAGACTGACAGCACTAATAACAAGTTATGAAGCTGAAAGAAGCT
TCTCAAAACTCCCAGGAATAAAAAGCAACCACTGATTAACCATGCTAGAGGCAGAACTGATTTGTCTTCCTTTTTGT
CTCTCTTAAAAATGATACTACAGGAGTTCCCGTCATGGCACAGCGGAAACAAATCCAACTAGGAACCATGAGGTTGC
GAGTTCAATCCCTAGCCTCGCTCAGTGGGTTAAGGAGCCAGGGTTGCTGTGATCTATGGTAGGTCACAGACACAGCT
CAGATCTGGCGTTGCTATGGCTGTGGCGTAGGCTGGCAGCTACAGGTCTGATTAGACCCCTAGCCTGGGAACCTCCA
TATGCCATGGGTGTGGTCCTAAAAAGACAAAAAGAAATAAAAATGATACTACAAAAATCATCAGATAAAGAGATAGT
TCAAAGTATGCAGCCAAAATATGAGAGGTACATCAGACAGCTGAGTAATACTAATTATTTTTATATTATTTTCACGT
GTTATGGTTGTTTTTCTGAATTTGGTCCTATTTAGAGTATTGGTCAGTCTGTGTTAGCTGTTGGGATGGCACCTCAT
ATTCTAAATGCAGTCAGCCTTCTGTATCCATGGGTCTTACATCCACAAATTCAACTAACCACGGATGGAAAATACTC
CAAAACATCACATTCCAGAAAGTTCCAAAAAGCAAAACTTAAATTTGCTGCATACAGGCAACTATTTGCGTGGCATT
TACATTGTATTAGGAATTATAAGTAATTGCAAGGTGATTTAAAGTATATGGGAGGGGAGTTCCTCCGTGGGCTAGCT
GGTTAAGGATCCAGTGTTGTCACTGCTGTGGCAAGGGTTCGATCCCTGGCCCATCAACTTCTGTATGCCATGGGCAC
CGCCAAAAAATAAATAAATAAAATATATGGGAGGCTGTGGGTTATGTGCAAATACGATGCCCTTTTGTGTAAAGGAC
TTGAGCGTCCTGGGATCTGGTATCCGTGGGGTCCTGGAACCAATCCCCTGTGGATACCCAAAGACGACTGCATTCAA
TCCCCAGCCAAATCATGTGTCTGCAAATTTGTGTTCCCTTTTCTTAAAGCAGGCCCTCGATATTGAATAAGCTTCCT
GCAGCACTTGGATGCCCCCCAGCTGAACCAGACCAGGCCTCAGGCTAAACGCTTTACCAGAGGTTTCTCAGATAAGT
CTCACAACGTCCTGTGAAGTCATTCTAGTGTTATCTCCACTTTACAGACATGCAAATGGAAGCTCAGAAAGGTGAAG
TGACTTGCCCAGTGTGTCACACAGCATAAAGTGATGGAGCTGATATTCAGGTCCAGAGAGCTGGCCTCAGGGCCCAC
CCTTTTAACTATTCTCAGTAAACATGAAGACTCACCCATGGACTAATCACCCAGGGATCTTTGGCACATCCTCTCAT
TTTGCCTTTCACGATGATCACTTAGCAATTGACCCAAAGCTAGCCAATCATGGGCTAGACTCAGCAGGGGCCAGCTT
CTCCTCGGCCCAGCTGGCGAGCATTGGCTCAACTCCTCTGCCATTTCCAGGAGCCTCCTGCGTGCCTGGTGTGAGCC
TTCCCCATGCACGCCATCCTATTCACCCCTCATCATGGTCAGTGCGGGGGCTTTTTAGCTGAGGAGACCGAGCTTTA
GCAAAAGCTGAGATCGCTGGGCTCCCCCACAAGGGGGGCGCTGAGTTTGAAAAGCAGACCCTCTGCCTCCCAGGCCC
AGCTCTTGGCCGGGGGATGGTGCTGGGGGGAAGGAGGGAGAGTCCTGCTTTATCTAAAACCTCTTTAAATTGGCTTG
CATTACAGGGAAATGCTCCCTGTTGGAAGAAACATGGTATAATTTGGGGGGCAGGGGTGGGGGGGGAGTAGTGCACG
GAAGGCTGTTTCCAGTTATGTTTTTCATTATAAGGGTCAAAGCAAACACAGACGCAGGAAGCTAAGAGACAAGCCTC
AGACTAAACATACGACCAGCTGTCGCTCCAGCCATCACAGACCTGTTCTCGGAGGGACATCTTGTAGGCCCCTTTCT
TGAATCCCCTTCAAAAATCTGAAGCCTGGATCCAGCCAGCTTCTCCTTGCTGCCTGGCTCAGAAATCATGGTGCAAG
AGTTTTTCCAAGAGAAATAGGGCGAGGTACATGAAGGATCGGTGCTGCCCTGAGAGGGCACTATGTCCGCCCCCAGC
ACAGGTCCCGGGCCTGAGACTCGTCCTCCTGGCCCCACAATGGCACTGTGTGGCCCACACAGAGAACCCCAGGCTGT
AGCCACACCCCGTGAGGTCCTGCCGGGCAGCCAACGAAAGCAGAACCAACAGTGACTGAGCCAGCATCCTGCCAGCT
CCCACTCCTAGATCCGATGCCGGGGACTGGAGGACTTTGTCTTCTTTCAGAACAACTGGGGGGAGCAGCAAGAAGTC
AGGGGGAGAGGGGGGCTCCTCTCTCCACGCTGCAGCCAGCTCATGATACCCACCCCCCCGGTGACCCCAGCAAAGCG
GAGGCAAATCATTTCAACGTTTCACGTACCTCATCCTCTGCTTCTCTCCCCCCAGAGTAAAAGGCGAAGCAAGTTCT
AGTGAGCTCTGCTCTGCAGAAGGAGGCAGGGCTGGGAGGAAGGGAAGGTGCTGCGTTCCAACTCCTGTCAAAAGAAT
AAACAGCGGTTTCACGAAGAGGAGCGCAGACGGATCCCACAGCAGCCAGGGGCCTTGTTCCTCCTTGCTCGCCCTGG
GAAGTGGGCTGTTTATCAGGCCTGTTGACTCAGAGCTGCATGCCAAGGCAGAGACGTCTCTCTCCGGCCCAGGATCG
GCCCGGCCTCCTTCACTAAGCGAAACTACAGGTCCAAACTAGGCCTGGTGGTGGAGGAGGGACAGCCACCACCCTTG
GGAGAGACACACAGGCCGCCCACATCACCCACTCCTCGGCGAAAATGAGAACCATTCTGAACCCAAACCACCCCAAA
TGACAACTAGCAGGGACAGCCAATGGAGAATTTAAAAAGAAGGGGGCAGAAAATGGAGAGGGGTGGCTAAAGGAGAG
CATCCTCAAAACTCCCGTTGAAATGCTACCTTCCGAGCCTCTTGTTCGCATCCTTTAGGCTTCAGAAGTTGTTCTGT
TTGAACACTATTTTTATAGAATGTTCTGAGATCTCCTGCATGGCAAGCCAAGCTATAAGAACTTCAAAAGGTCACTG
AGGCCCAACCCAACTCTTTGGCTGAATAATGCTTAACCCTCCCCACACCCACCTCCTGCTCCCAAAATAGAATTTCC
TAGCTGGAAGAGACCTCACAGCAGTGGATTTGTAAATGTCGCAACAGCTAAAGCTTTAAAAAAAAAAAAAAAAAAAA
TGAAGTCATTCTCAGAACCCCACTATGTAAAACAGAGGACACAGGGGGCTTTGGCTGAAGGAGGGAAATGAAGTAAG
TAGGGGCTCAGAGCCCCCCCACCCATTCTTCCCAAGTGGCCCCAGACACTTCCTGGGAGTAGAGCCTAGAAACCCCA
GACTAAGGAGAAGGGGCCGAAACCTGACAGAAAGGAGCCAAGAACTGCCCCCTCAGCTTCCAGCGGATGGATGCCTA
ATTTAGCTTCTCACTCCTGTTCTGGGGAAGAAATTCACCGCCCCCTCCTCTGGGGCATGAGCTAGTTGACCACAGTC
TTCAAGATCTGCTTAATAAACTACTGAAATCCTCCCTGCTGGCATCTACTAAAGCTGAACCAACCACACCTCATGTT
CCAGTCATTCCGCCCCAGATTAATACCTGAAAGCAAGTGCATTTAAGTTCAAACAGAGACGTGACCTGGGACCAAAA
GCTGGAAAAACCCCAAGGCCCATCATCAGCCAGATCAGGTGTGGTCCAGGTGAGGGTCACACACATCCGTGAGAAGG
AACCAGCCACAGCTGCTGACATCAACAGGGTAAATCTCACACATGGTACTGAGTCAAAGCAGCCCTGGATGCTTGCA
TTTATTTAACGTTCAAAAATAGACAAAACCGGGAGTTCCCGTCGTGGCGCAGTGGTTAACGAATCCGACTAAGAACC
ATGAGGTTGCGGGTTCGGTCCCTGGCCTTGCTCAGTGGGTTAAGGGATCTGGCGTTGCCGTGAGCTGTGGTGTAGGT
TGCAGACTCGGCTCGGATCCCACGTTGCTGTGGCTCTGGCGTAGGCTGGTGGCTACAGCTCCGATTCGACCCCTAGC
CTGGGAACCTCCATATGCCGCAAGAGCGGCCCAAGAAATGGCAAAGCCAAAAAAAAAAAAAAAAAAAAATAGACAAA
CCCAGGGAGTTCCCATGGTGGCTCAGCAGAAACAAATCTGACCAGTATCTACGAGAATGCAAGTTCGATCCCTGGCC
TCACTCAGTGGGTTAAGGATCCAGTATTGCCACCAGCTGTGGTGTAGGTTGCAGATGCGGCTCGGATCCCATGTTGC
TGTGGCTGTGGTGTAGGCCAACAGCCACAGCTCCAATTGGACCCCTAGCCTGGGAACTTCCATATGCCCCAAGTGTA
GCCCTAAAAAGACAAAAAAAAAAAAAAAAAAGACAAAACCAATCTGTGGTGCCAGAAGTCAGAGTGGGAGTGGTAGA
GACTGGGAAGGGGAGGCTCAGAGAGCTGCTGGGGGAGGGGGGGGGCTTGTCATGTTGTTTCTCGAGCCAGGTAGTGG
TTATGCAGGTGTGTCCACCTTGGGAAAATGCCTCACAAACATTCCCTTTCAGTGTGTGTGTTAAAAACAAAGATGCA
CAGAAATCTTCCTGCTGGAAGCTGCCTTCTCTTGGGAATTCTGACTTCCCCTGAGTCTACAGGGTCTCAGGGCCACA
GGGTCATGGATAGACCCCGTTTTTTCCTTCTCTTGGGTTCAACGCCCCAATACCAAGCACCACAGAGCACCTAAGTA
CGGACTCAGGGAAGATCTTTCACATTAAATGATGCAGGCAGCTGGACTGTGGTCAACTGGGAGGGAAAGTTCACAGC
ATTTGGAGGCTCAGGAACTGGGCTAAGATAAACTGGTCCTTTCAAGAAGCAAGCACCCAGGAGTTCCCATCGTGGCT
CAGTGGTTAACAAATCTGACTAGGAACCATGAGGTTGCGGGTCCAATCCCTGGCCTCGCTCAGTGGGTTAAGGATCC
AGTGTTGCCGTGAACTGCGGTGTAGGTTGCAGACGCGGCTCAGATCCCACGTTGCTGTGGCTCTGGCGTAGGCTGGC
AGCTACAGCTCCAACTTGACCCCTAGCCTGCTGGGAACCTCCATATGCTGCAGGAGCGGCCCTAGAAAAGGCAAAAA
GACAAAAAAACAAAACAACAACAACAAAAAAAAGCAAGCACCCATCATGGTTGCCACCTTCCAGTTTACAAAGCAGC
CTCTCTCCTTTAACTCAGCAAATCCTCAGGCTCACCCGCCCCGGGTCAGGGAAGGGAGGGAGGCACTGGGAGCCTCT
GTGACTTGCTCAAAGTTGCCGGCTGGTGGGTCTGATGCTGCCCTTCCTCCTGAGCTGCCTCTGGGGAACACCCTACA
GGTTCGTGGAATTAGAGGCTCCAGGCTCATGAATCAGAGCACGACAGAGTATGCAAACTTGGAAGGCAGAAAATTCA
ACTTCCAGAGGATCCGACATGACCTTCCTCCTTCTCCGACATACCCTGATGCCCAGACTCTCAAAACAAGGAAGCAT
GTACTTCCGGTCATTCCTTCATGGAGAGGCAGGGAACTGTAGCAAGTGAGCCTCAGGTCTGCTGATCAAAGGAGGCC
AGTGGCCATCCAGGTAGGAGTTTGGCACGTTTCCCAGCCCAGCCAGGCCGACTAATCTCATCACTCAATGTTCCCCA
AGGCCCCTTCCAGCCCTAACAGTCCATAGGCCTGTCAGATGACAGCCAGCATTCAGAGCCTGTCCATCTGCCATGTC
CCCTGCAGAGGAGTGCAGGGCCTTGGAGCTGCGGCTCAGCAGCTGCAGCCCAGGTGTGAAGGGTCCCGGCTTCATGC
CCCAGACCCCTTCCACCTGAGAAACACAAAGGTCCGGATTCCCACCCTGTGGGAGAGGGAGAATTAAGTGTTCTTGG
CAAAAAGTGCTACAGATACAAAGATTGCAGCTGTCACTTTTAATCCTAAATACGTTTAGGGCAGGTATAAGACATTC
TTGCTGTCACTTGTGAGTGATGGAGCAGTTTAGTTGGTTTCCTCTTCCGTGTGGTGAGGATAATTATAATCCCCACC
GCTCGGGGTGGGTGAGGGGCCTAGAGCACCGTGGTTATGAATGTGGACTCTGGGCCCAGGCTGCCGGAGTTCGAGTC
CCAGGCCTGCCCATGTGCGATCCTGGGCAATGTGCTTAACCTCTCTGTGTCTCTGTTTCTATGGCTGCACAATGGGA
ACAACAGCAGCTGGATGGTAGCTGGCACATGGTAAGTGTCTAGAGATACGTATTACCCGATATTGCAAGAATTAAGG
AGACACGCCCGGAAAAGTGCTTGAGGTGCTCAATCATTGTCCGTCTCTGCTGTTCTATTAATCCGAGGCTGCAGCTC
CTTGGAGTTTACATTTGTGTATCAAATAGTCATTTTGACCACGTAACCCTGCAGGTGGGGAAAGGTACGGAGGGAAG
GGTTCCTGGCACGACGTTTCCGTTACTGTTAAGTACTGCCCCCCACACACGCCTGTGAGTATCAGAGCTGAAACGAT
CTTGGCAAAAGCCCACATAATAAATAACGGCAGTCAAGAGAGGTTGCATCTATAAGTCTATTTCCTTGAGAAGAGCT
GGAAAAATGAAATCATGATGACTCTTCCCAGGCCAGTACATTGCTAATCATCTTGAGATCTGCCTCTGCCCCAGGTA
ACTCCAGGACAGACTCCACCAAAGCCATGCTGAAGCACTCCTGCCTCTGCAAGCATCCATCCTGAGCCTCAGCCCTC
CTCCTGCACACCAGGAAGTCCCTCTCTGGGGCTCATGTCAGTCCTTCAAGCTCTATAGGTCAGACTCTTCCTAGAGA
AGAAAGAAGCTGGCTTTGTTGACAGCTGGGGAGATGTGAGGCGCTCCCACGGAAGGGCGAGGCCCGGGTACTGATGA
CACCCTGGGCTTGAGCACCAGCACAGGTGGCTGGAGGATTTCCCCACCCAAGGAAACCGCTCTATTCCTACCCTCTC
TTGGTCCTTCTCACCCCTTCCTCAGGCCAAGGACCCCAGATGGAGGTGAGAAAGAAGCACCTGCTCCTTATTCACAA
TTGGGCAGTAGGTGCCAGGGGGTACCCTTGCCCCCGACCCCCCACAGAAGTTCTCACTCTTTCCTCAGTAGAGAGAA
CCTCAAAGTCAGGTAAGTCAGCTCCCTGCCTCAAAGCAGGACTGCTTTTTGAACACGTGATAAGCTCATCTTCCGTC
AAGGTCACACCCACGCCCCGTTTAGAGCCCACTGCCATCCACAAAAGCCACATAACATAGAGGCTAAGTAGGAGAAA
TATTACAAGCCCAAGTTATAAGAAAGGGAACTGAAGATCAGGGAAGAAACTTACAGAGTCGTATGGTCTGAGTCAGC
AGCCCTGGAATGGAAGACAAGTTTGGGGTCTTTCTGTGAGTCTGTCCCACCTCAGCCTCGTACACCCCTGGTGGTGG
TGAAGCCAGACCAAGCTGGGGATGCTAACGGAAGCAGAACAAGAAGAGGGTCATGAACCAGATTCCACTAGAACCCA
AGTTCTTTGGGGGGTGGGAGGGAGCACTTGTCTTCTGTCTTGGTCACTTCTGGGCTTTCCTGGTACCTGGAACAGTA
TTTGACATCTATCAGACGTTCAGTAGATATTTGCTGAATTAATGCTGAGTGAAAGCCTACAGGAGCCAGGCAGGCAG
CAGAAGTATGTGAATTTGACCAGGTAAGGATGGACTGTGATAAACTAGCCAAATCAGATCAAAATCAGATTTTAAAA
AGAAAACAGGTTTCCCATTGTGGCTTAGCAGAAACGAATCTGACTAGTATCCATGAGGTCTTGGGTTCGATCCCTGG
CCTCGCTCAGTGGGTTAAGGATCCAGTATTGCCACCAGCTGTGGTGTAGGTCACAGACACGTCTTGGATCTGGTGTT
GCTGTGGCTGTGGCTGTGGTGTAGGCCGCAGCTACAGCTCCAATTCAACCCCTAGCCTGGGAACCTCCATATGCCAT
GGGTACGGTCCTTAAAAGACATAAATAAATAAATGAAAAAAGAAGTACCCTTCTTTGATTACAGAATGTGATATACT
GGCCATAGATGACTCCTCTTTTAAGGGAAATTGTTTTGTGCCAGAAGCGAAAAGTATTGTTTGAACCCTTGCTCCCC
AACCTAGGGGATGTAGGCGTGTCTGTCCCTTCTCTGTGCGTCTGTTTTCTCATCTGTGAAGTGCAAGGTCCCTCCCA
TTTCCACTCCATCCTGCCTGGGCCTGAGTCTGAGGGTAGAGTTGTGAACTGGGCTCCTATAGCAGTCTGACTGGGGG
ACTCAGAAGGCTTCATGGAGGAGGGGATGTGACCAGACCTTTCCAGATGGGCTTCCCCTGCCTCCCAGGGATCTGGC
ATATCAGCCTGCACAGCCACTCACCCTTCTCTTCCTTCTCACTGAAGACAGGCTGAAAAACTAACCTGCCGGGGGAG
GCAGGCAGCCCCACACTTCAGAATTTATAAATCCTCCTCTGCTCAGGCTCAGGCCCAGTCCATCCTGGGAGGTGCTG
GAGGTCATTTTATGAACCAACCACCTTCGGCTTTCGGGGCGTAGGGATGGGGCAGGATGCCACAGAATCACCAGCCC
ACTCACGAGCCCCCCTGAACCCTTCCCAGGGTGACAGAAAAGAGGAAATGGAGCACAATTCCGGCCCCAAGACAAAG
AAACTCGGCCAAGCAAAGAGAAGGGAAACAGCTTCCTGAGTCAGGGGACTTGGAATCTGCTAGGGCCACAGGGAACC
TTCCCCCCATCATGGTGAGGCTGAGGTGTGGACTCAAGCAACTGAGAAGATAAGGACAGGTGGGTCCGCCCCCACCC
AGCTCAGCCCAGAAGCATTTCTTTCCAAAGCGCCCGTGGAAAGGAGTGGTTTGCAGTGAAGAACATTTTTCAAAAAA
ATCGAAGTCTAATACTAATAATATAACCAGATAAAAGAAAGGCCAAGAAAGTGCCATATAAATCCAAAGACACGGTT
CCACAGGCCACGTGGCCACAGGCACATTTTTCCCCTCCTGGGCCTCACGCCCCGTGTGGGCACTGACGGAGTCGAAG
TGGAACATTCCCAGGACCCACCTGGGCTCGGTGGCTGTGAAGAGCCTGTTGTTACTTGCTCTGCAAACCTGGCTGAT
GAACATGCAGCCTTCAGAGCGCAAGGTCACCTCCTCCAAGATCTGCCTCCTGGCACAAGTGGATTCTCACAGCCCTG
GTGTGGCCTGCTGGTTTCACGGCACCTAGAGCGCAGGTTCTTGGACATATGTCCATCTCACTCTCTGCACGCACATT
CTCAAGGGCAGCAGGGAAGTCTGCTTTAGGTCAAGGTCCCTGGTGGTCCTCACCACAGGGTCTGGTAGAGAGGAGGT
CTTGAGGATCAGTAGGCTGGTGACAGATGGACAGATGGACTTGCTGGGGCTACTGTAATAAAGCACCACAAAGTGGG
TGGCTTAACACAGCAGAAGTTTATCCTCTTATACTTCTGGAGGCCAGAAGTCCAAAGTCAAGGTGTTAGCAGAGCTG
GTTCCTTCTGAAGGTCATGAAAAGGAATTCTACAGGCTTCTCTCCTGGCTTCTGGTGGTTGCCAGCCACCCTTGGCA
TTCCTTGGGGCAGCATAACCCAACACCGTCTGCATCATCACACAGTGTTCTCCGTGTGTGTCAGCCTCCAAATTTCC
CTCTCTTTAGAAGGACAACAGTCACTGGATTGGAGCCCACCCGAATCCAGCATGACCTCATCTTAATTTGAGTCATC
TGACAAGAATCTATTTCCAAAAAAACTCATATTCATAAGCACTGGGGATTCGGACGTGAACCCATTTTTTTTTTTTG
GAAGACACAATTCTACCCACTAGAGACCGTTTCCCAAATGCCTATTGGCTGGGAGCGTGTAAACACTAGCAGAACCA
CCTGTGAGGGTGGAAACGCTGCATATAATTACGGAGTTGAAAGCGAAAGTTTGGAGGCAGGCGGGGAGGTAGGGGTG
GTCTTGAGAAAGAGGAAAACATCTTAGAGCATCTCTACTTGGCCAGGATTATAGGAAGAAGAGAAATGCCTCCCCGG
GACAGGCATCTGTGGGATGTCCCGCCGAAATGCTGCCGGTCTGTCAATACTCAGCTCTGGGCATCACAGAGCCATGA
ATGGGTAAGCTTCCTCCCAAGAGGAGCAGGATGTGAAAGAAGAGGGGGCCCTGGGGCAGCTGGAACCAAGAACCTAT
GGAAGCACAGAGCTGGGCACCAGATTGCAGTGGGTCAAGGAATGAAGGTCAGGTGAGAAAGTGACGTGCAAGGACCT
CTCGCCAGCAGCTTGCCTTGGGAAGGGCTGGAGGGAGGGTGCCAGCTAGAGACACATGGAGCAAAAAGGAAATACCC
TTGAGTACACTGCTGATAATGAAAAGCCCTTAATGAGACAGAGCCGAGGAGAGGAGGGTTTGAAGATTCAGAGGAGG
GAGAGGATGGGGGCTGAAGAGCATCTCTTGGCGGGGAGATGGGGGTGCCACCAAGACAGGCTGAAAGTGCTCCCCCT
TTTTGAAAGGAGCAGGAGACAGAATGGGTGGGTTGGCAAGTCTGGGGATAAAGCGGGTAGGTGACAGGCTCCAATCC
AGAGCAGCTGAAGCGAGGAGGGAGAAGGGGGCCAGGAGGCAGAGAAGCTGGAGAGCTGTGCAGAATCTCATCACCAG
GAACCTTGAACTTGCACCTGAAAAATGGGCATTTCATCCTGAAAGTACTAGAGAATCCTTGAATGCCACTAGGCAAA
GAAAGTTACACGATTTGCTTTTTAGAAGACTTCCTTGGCTGAAGGATGAGGGAGCCCAGCCAGGAGGCTGCTGGCCA
ATGTCAGAGGAAAGAGTAGAGACCTAACCCCACAGGTAGAGCTGGAAGACAAGAAAGAAGTGGCATCTTGAGACATA
GGGTTACATCTATCTTACTTTCTTTCTTTCATTTTTTTTTTTTTTTTTGCTTTTTAGGGCCACACCCACAGCACATG
CAAGTTCCCAGGCTAGGGGTTGAATCGAAACTGTAGCTGCCAGCCCACGCCACAGCCACAACAATGCCAAAGCCGCA
TCTTCGACCTACACTACAGCTCACGGCAACGCCAGATACTTAACCCACCGAGCAAGGCTGGGGATCGAACCCGCAAC
CTCAAGGTTACTAGTCGGATTCCTGAGCCACAATAGGAACTACCGGGTCACGTCTTTGAAAATCTGCTTCAGTGTTA
CTTTAGAGAAACTGTCCTGGATTTAAAATTACTTTCCTTTTGTAGTTATCTATCTTTCAATTTTATTTCTTCTTCTA
CCAGAGTGTCAACTCTGTGGGCAGATATTTTTGTGCGTTTGGTACCTGTGTGGAAACATCTGTCTATTACAGCCCCT
GGTGCTCCGTACAGCTTTGTAGGCTAAAATGCATGCCTGGTACAGTGCTTGGCACCTGTGTGTTCAATAAACATGAA
CTATGGTGATAACAACAGCAAGAATAACAGTGAGCAATGGGATGAAGGGAGTGAGGCAGAAATGAGACTAGTTTGGT
GGGACTCAAAGTGTGGACTGAGCAACCGGTAGCATCAGCATCACCTGGGAGCTTGTTAAGAAATGCAGAGCAGCAGG
CCCACAGCCCAGGAACCTGTGTCTGCATGAGGTCTGCAGGTGGTCTGGGAATGGGGCTGGTTCCCAGGTTTCTGGTT
GAAGGAGGAGAGTGGGTGGCATCGCTGCTGACTGACATGGAGCGGCGGGGCTGAGAGGAGGGGGAGTCAGTGAGTTC
TGCTCAAGAGGTGCTGAGTTTGAAGAACCTGCAGAAGTCAATTCAGCAATGTTGTCCCAGAGAGAGAGCCCGGGGAG
AGCCCAGTTTCGGAGCTGCCAGCCCAGCGTGCAGGCAGGAGTCGGCAGGTCTTCTGTGTGCCAAGGGAAAGGAGCAC
GGAGAGCAGAATGGGGCCTCCTTAATGGGCACCGCCTTGAAATCTGAGGGGCAGGGCCGAGAGGCAGGAGGAGAAAC
AAGAACAAAAGTTGTTGCTGGGAGAAACCCCATCTGAATTCTCAGCTCAGCTCCACCCGTGACCGCCTCTGGCCCTG
CTTCCCCTGGAAGAGGGAAGGCCACGGACAATTGCTCGGGCAAGGTTGCTGCTGTTTGAGAATCCCAAGGAGCGGGA
CTGTCAGGCAAACAGAGGGGTGGCAACAGAGAGGGGTCCCGTTTCCAGCTGTACCTCCAACTCCGGCAACTCCCTGC
GTGCCTGGTTGATTCCCGCCCCCTTCGGATGACAAGGTGGGGCCGGGGTCTCTGACCATGTTGCCTGCCAGCTCTCT
GGGCTCACCCCTCATGTCCGGCCACAGACTCTAGGGGAAGACCCCAGCAGAGCATAATGGCAGCTGCCTTCAGAGCA
CGTGAGGAGGCTCCAGAGGCCAGACCAAGAGGTGAGGGAAGGGCACGCAGGGTAGGAAGCCAGGATTCCCGAGCCAA
CAGGTGTGCTCTACCTGGCTCCCATCAGTACAGCTGAGAGTCAAGGTCTAAAGAAGCCTCTCTGTCCCTCAGCCAAA
AAGGGAGGCCCAGGAACCAGCAAGGGCCACTCTCTGCATTTATCAGGTCCTAGTCTGGCGAGAGGGACACGTGCTGA
CTGCAGACCGCAGCTACTGCAGTTGTGTTCAGTGGGCTGGGGCTGGCAGAGTGGGGCTGCACAGGTGTCCCCCGGAG
GAAGTCCCAGCTCCTCCCTGCCCCATCACCTGTTGTATTTTGCTTTACCACCCTCCCATTTTTGCCATTTGTGCTTG
GCCTTGTCACAGCAACCCCTCCTGGTGCAGGTAGTTTCCCAGGGCCTCTAAAATCAAGGTGCTTCCCCTAGAACAGT
TCTGATTTATACTTGTTATGGCTCAATGTTTTAGTACCTCCTTTCACTTTCAAAGGTGTGCAGGTGTGGAGGACAAA
TCATGTTGCCTGTCACCCTACATAAAAACGGTTCAATAAAATAGAGTTCGATGAAGTCCCCTTCAAGACGCCTCTCG
GCTTGGACCCTCCAGGAGTCAGGGCTTGTGTTTACCAACAGCCGGTGCCGTGACCTCCCCCTCTCCAGCATCCTTCC
TGCTACTGCCTGTGGTACAAGAGGTGGTAAAAGCCTTTCTGCCACCCCTCCCCTAACCTGTCCCCTTCAGTGCCTGT
TGCTGGGATCATCTCAGCTCCCCCTGCCTCCCTGTGTAGGCTGGGAGGAATTAAAAGTCTAAGAATTTACTGGAAAA
TCCTAAGGTTGTTTTGTCTTGGGCTTTTTTCCCCCCTCACTAGATTTTTTTCTTGTAACAAGTTGACGAGCATAAAA
GACCTTCCAAGAATTAATCTCTAATCATGAGAGATTTCCTTCCTAGTGGAAAGCTAAAAATAACAAAGACAACAACA
ACAACACCCCAAAACCTCTTAACTGAGCCCACAATGGAGATGGCTTTTCCTCTGCCTGTTCTTTGTCTTTTGCCATT
TTTTTTTTTTTTTTTAAGGGCCGCATCAGCGGCATGTGGAGGTTCCCAGGCTAGGGGTCTAATTGGAGCGACAGCTG
CCGGTCTACACCACAGAACAGCAACGCCAGATCCGAGCCACGTCTGCGACCTATACCACAGCTCACGGCAATGCCAG
ATCCTTAACCCCCTGAGCCAGGCCAGGGCTCGAACCCGCAACCTCATGGTTCCTAGTCGGATTTGTTCTGCTGCGCC
ACGATGGGAACTCCTTTGCCCGTTCTTGGAAAGAGCCAGGCCCCAGTTCAAATGCCAGTGGCGCCCCACCCCCACCC
CCCACTTTCTTGCTGCGAAGCCCTGGCTCAGTCACTTCACATTCCGAGCCTCAGTTTACTCATCTGTTAAAGAGGGA
TGATAATTCCTTACTCCTTGAATTGTTGACAAGATGAACAGTCTGTAAAGCTCCTGGTAGGTACTTGGGAAAAAAGC
AACTTGTATTATTATCGCTGGTCCCTAAGAGACAAGCACTGTCCCCACCTCATCACAGTGACAGGAGGCAGTATGCC
CAGAGATTAGAGCTTGCACTTGAGCAAGACAGGCCTGGGAACTGACTAAATGCGTGACCTTGGGCAAGTCACTGGAC
CTTCTAGGACTTGCTTTTTCTCCTCTGTAAAATGAGAATAACAGTGACTCACCATCGGTGAGATGACGCACATCAAG
CTTGGCATGACCCCTGATGTTGCAGCAAGTGCCCAATAGATGGTAGTTTCTCAATTCCCAATAGTGATTATTGCAGA
ACTCTCCACCTCACAGGCTCTGGCACCACCTGCTCTGTATCTCCAGGGTCCACTATGTTCCCCTGTCCCCAAAACAA
CAGCCCTTCCTGTGCAGGGGGCATTTACAAATCCACCTTTCCCCTTCCGCTGGAGTCTGAGCTGCAGCCCGTGAGTC
AGGCTGGGTCTCCACGTGCGGAGGAGGAGGTGGAGGAGGAGGAGTCTGGTAACTCCCCAAGGGGGGCTCAGCTGGGA
CTGGAAGCTGGGTTTGGGTGCAGCCAAGAATTTCTTCAGCCCCTTCCTGTCCCACAGGGAGCCTGATTCAGAGTTGA
AGGGAATTACGTGTTTGTTTATTTATTCATTAAATAAATATTTAACACCAGGGAGTTCCCATCCTGGCTCAGCGGTT
AGCAAACCCAACTAGCATCCATGAAGACATGGATTCCATCCTTGGCCTCGCTCAGTGGTTTAAGGATCTGGCGTTCC
TGTGAGCTGTGGTGTAGGTTGCAGATGCAGCTCAGATCCCGAGTTGCTGTAGCTGTGGTATAGGCCAGTGGCTACAG
CTCCAATAAGACCCCTAGCCTGGGAACCTCCATATGCTGCAGGTGTGGCCTTAAAAAGACAAAAGAAGACCCCTCCC
CCCCAAAACTTAACACCAATGTTGATACCTACCACGTGCCAGGCACCATTCAGGCTGCTAGGTCAATAAGGATTAGC
CTATTCTGTGCCTTTCTCACAGAGCTAGTGGGAAGTGGAGCCCTTCCTGGTGGGAAGCTGAGCCCGGACAGCAACAC
TTCTACATCCTGAAGCCAAGGTGAGTGTCCTGTGACAGCAATGAGTCAGCCCCTCTCTGGGCTCCATGGACTTCTGG
AAGACTCGGAGAGCAAGCTCACCTGCCTCCTTGCCCGTGTGGCTACAGGAACATGTTTACCACCCAGGGTCACTCTC
TCTCAAGCATGGCCCCAATCTTCTGAGCTGCCTCACTTTCCAGATGAGAAAACTGAGGCACCAAGGCAGGGAAGTAA
CTTATCCAGGGCCACTTGGTGATGAGGTGAAGAGGCCAGGGCTAGTACCCAGGTATCTGGCATCTCTCTAGGCTGAG
ACGCCTATTAGCCACAGCACCAGAAATCAAGAGCTTAGAGACGGGGCGAAGGGCTGCAGTCAATGGTCTTCTTCTAG
AGTTTTCTTATTAATGCCCAGGAAAACCTCTGATGGGACATAGAAATGCCACTGGGAAAAGGGGAGCATCGTGTGTT
TACTGGAGACAAGTGAGGCACCCAATTCAAAAAGAAGATCCCTCTCAAACATAAAATAGTTCAGCAATGGAGTAAAA
AACACCTAAATATGTGTTCCACTTACAAAGCATCCTATGGGCTGTGATGAAGAATGTGGTTTGGAAACTCCGATTCC
ACCCCATTGCCTCTGCCTTCACCTCCCACCCCAGTGTTTAGCACCAGGAGCTCCCAGCACATATCACCTACCCTTTT
CCTGGCTGCTGTCTTCTTCAATGAGCTTCTGCTTTTGATTCCCCTAGAGAGGCTGGCAGTTTCGGGCACCTTTTTGT
TCCTCTGCTTAGCAGTTGGGGCGGAGAAGAAGTGGCTTTGGGGTTTTTCTTCTCTGGGTGTGGTTTCCTAGCCCTCA
CAAAGGAAAGCCTACAGCCTGCTCTGTCTGCACCACCAGCCTGGTTGCCTCAGCTGGCAGAGCTGATTAGCATGCGA
GGTGCAGAAGGGAACAGCCTGCCTGGGGTACTCAGGATACTGTTCTACTAAATGTTTCCTGCTCTCCACCTTCATAG
TAGGATTTCATTTCCTGGTCCCCTTGCAGTTGAGTAGGGCCATGTGACTAGTCTGACCAATAAGATGTGAGTTGGCC
CAAGTATTTAATTGCTGGTCAAAGACCCTCCAGGGCTCTCTTTCTCTGTGCCATGAAGTATATTCAAGGACGTAACT
GCTCCATCAGCCTGGCTCCTTGAATGAGGAGCACAGCCCCTAGCTGACCCACGGGGCTCATGTTAATTAGAGTAAGA
CATAAACCGTTATGGGTTTGGCCCCAAAGATTTAGGGGCTGTTTGTTACTGTAGCATAACCTACACCATCCTGACTG
ATACACTGCCCATCTCACACAGAGTGAGATATTCCCTAGTTAAGTCTACCATCTTCCCAATGTTGCTCTTTCAGCCA
GAAGCCATTTCACTTCCTCTGAGCTCCCCTTGGCCTCCTGTCACACTTCTGTTCTGCACTCTGACTTCTACTTTTAG
TCCCTTATATATAATTACATACAGCCAATTTCACATTGTGAGCGCCTGAAGAGCAGGAATCTGTACCTTATATTATG
ATGATGATAATAATAATAATAATAAACAGAGGCAGCAAATGCTACTATTTATTGAATGCTGGGCTGGGTTCTAAGCA
CTTGACATTCATTCAGTTCTCACTAAGCTCTGAGAGGTCAGTACTGGAACTACCCCCACTTTACAGATGAGGAAGCA
TCTCAGTTTGGTTCAGCTGAAATTGAACCCCTAATAATATATATATATATATATATATATATATATATATATATGCA
TTTTTTTTTTTTTTGGTCTTTTCCTAGGGCCACACCCGCAGCATATGGAGGTCCCCAGGCTAGGGATCTAATCAGAA
CTATAGCTGCTGGCCTACACCACAGCCACAGCAACACCAGATCTGCAACCTACACCACAGCTCACGGCAACTCCAGA
TCCTTAAACCACTGAATGAAACCAGGGATCAAACCGGCAACTTCATGGTTCCTGGTCGGATTTGTTAACCACTGAGC
CACGACGGGAACTCTTAATATTTTTTTAATAAATATAGTTCAACTTAAGTCATTCCCTCTATAATCCTAGTCACTTA
TTTTTCACATTTAAAACATTCCCAGAAGGGGTCTATAGGCTCCCCCAGATGCCAAAAGAGTCCATGGCACAATAAAG
GTTAAGGTCCCCTGTAGAAGCAGATACCAGGGTTACAGTGACAGGGTTCTGTCCCCTGTTCTCCTGGAACCCAGAGT
TTCTGGCTGGTGGAGGGTAAGGGACCCTACACCAAATTCATGCCACAGTGGGGAGTGAACAGGAGCTACTTTATTGT
ATTCACATAGCATAAACATAAATATCGTAGGTTTGGCATATGGAACTCCCTGTCATGAATATTTTGATTTCAGCAGT
GTCAGCCCAAGTATAACATTCATCACAGTAAAGAAGTCACTTGTTTCCCCAGTAAAAAAACAAAACAAGGGCGTTCC
CTTCATGGCTCAGCGGTTAACAAACCTGACTAGGATCCGTGAGGATGCAGGTTCGATCCCTAGCCCCACTCAGTGGG
TTAAGGAACTGGCGTGTAGGCCGGCAGCTGTAGCTCCGATTCAACCCCTAGCCTGGGAACGTCCATAAGTCGCAAGA
GTGGCCCTAAAAGGCAAAAAACAAAACAAAACAAAACAATTCCTAACATCCAGTGTGCTAATTAGAAAAGCATCAGC
TCTTGATCACAAATTGGGATAACAGGACAGCAGCCATCTCTGGTCAGTCCCACTCCCAGACGATGCATCCTTGAGGG
CAGATGGGCCGACCACCCACGATGAGACTTGCTTTCTTAGCTTCTGAGCACTGGCTTGGTCCAAGTAGCACTCACAT
AATCTCCCATATTGTATATGCTGAAGTTTTATACTTTATTGAACCAGAATTTACTTTAAATTCCAGGCATCCAAACA
TATACACTGAATCCAGGTGAATCCAAGCAGAACTCTCTGGATTTCAGAAATCCTGGGTGATTACAAGACTCAGGGAT
AAGGTAGCAGAGCCAATGCTCTGTGCCTCCTTGCCAGCTGGCCAGTAGTGAGGGCTGAGCCCCAGGACAACCGGGTG
GCAGTCTGGCACTGCCCTGGTGGGCTGGATGACCTTCCGCAAATTACAGGCTCAGTTTTCGTATCCTCCAAATATGG
AGCCATACTAGATCCAAGTCCAGGCAAGAAACAATCACAAGGCACCCGCGCTACGCCTAGTACTGTGGGGAAAACAG
AAATTACACAAACTCCATAAGGAGCTTACATTCTAGTTGGGGAGCCAGGCCTGGAAACAATTTAACTATTGTGCACG
ACAGAAAGAAGTAAGTATGAAGGTGGTGGAAGCCCCCTCTTGTGCTCTGGGACCACAGAGGAAGCACGAAGCCAGGC
TGCATAGGCCTGCGCAGCTCGGTTTCAAAGAGGAAGGGGCTATGCTTGAACTGGGCTTCAGAGGGTGAGTAGGAGTC
TGATGGGTGAGGAAGGGCATACAGGTGGAAGGGCAAGGATCTGCAAACTCGGGGTCTGGAATGGGAAGCCCCACCCC
CAGCCCAGATCCCAGCCCAGGGGTTCCAGTCCTGCTCTCTCCACACATCCGCTGCTTTGGAATCTGGAAGAGTCCTG
GAAACCTGTATTTTGAACAAGCTCCCACAGTCATTCTCACAAGCAGGCAGTGAGTGTTATAGATTGAGAAAAATGAA
TGAACAAATGAATGAATGAATACAAAAATGAACCTGAGAAGTTCCTGTTGTGGCTCAGCAGAAACGAATCCGACTAG
CATCCACAAGGACGCAGGTTCAATCCCTGGACTTGCTCAGTGGGTTAAGGATCTGGCATTGCTGTGAGCTGTGGTAT
AGGCTGCAGGCTCAGCTTGGATCCCACGTTGCTGTGGCTGTGGTGGAGGCTTTCAGCTGTATCTCTGACTCAACCCC
TAGCCTGGGAACTTCCATATGCTGAGGGTGCAGCCCTAAAAAGACAAAAAAAAAAAAAAAAAAAAGAACTTGACTTC
CGGTAAGTCCCTTTCTCTCTTAGGATGTCCACACTACATTAAGGAGCTAAAGAGCTTCAGTTGTGGCTCAGCAGTAT
CCATGAGGATGCAGGTTCGATTCTGGGCCTCGCCCAGTGGGTTAAAGGATCCAGTGTTGCTGTGAGCTGCAGTGTAG
GTCACAGACAAGGCTCAGATCCTGTGCTGCTGTGGCTGCAGCTCCGATTTGACTCCTAGCCTGGGAACTTCCATAGG
CCACACCTGCGGCCCTTAAAAAAGACAAAAATGAAAAAATAAAAAGCAAAATAAAAGTGCTGAATTGGCCTGGTGGC
TTTCAAACTGTGTTCCAGAAAAACCCCAGAATCTCCCTGAAGTCCCTCAGGGACACAGAGGAACTGGGGAGGCTGAG
AGAGCCGGACTCTGGGCCCCATCCACCCTTCTCAGATTACCTCTCCTTTTATCTCTTTGCTCTTTTTTTTGCAATAA
AGGGTTCTTGGCTACAAAGAACTCTTAAAGCCACTGAATTGAATAATCCTAGAATTCCCAAGGAGTCAGAGTTCCCA
TTGTGGCTCAGTGGTTAACAAATCTGACTAGCATCCGTGAGGACGCGGGTTTGATCCCTGGCCTCACTCAGTAGGTT
AAGGATCTGGCGTTGCCGTGAGCTGTGGTGTAGGTCGCAGACGCGGCTCCGATGCTGTGGCTGTGGCCAGCAGCTAC
AGCTCCTATTCAACCCCTAGCCTGGGAACCTCCATATACCACCAGTGCGGCCCTAGAAGACAAAAAAAAAAGAATCC
CCAAGGAGAAATTTAAAAATTTCTTGAGGGCAGCAGCTTACCTTTGGCAAGTATGAAGAGAGCATAAGGGTCTTTTT
CAGAAGCAAGTTATTTAATCATCACATTTTAAAAACCTTTTGCTGTGGCCCAGAAATTAGTGAGTGAAGGAAAAAAG
CAATGTGGTATAATAATGCAAGGGAATATTATGCAACCTTTAAAGAACACTTTTGAGGAATGGTTAATACAATGGAA
AATAAAGTGAGGAAGTCAGATACAAAATTTCATACAGACTGTGATTTACGGTATGGATTTTTTTTTTTTTTTTTTTT
GGCTACACACATGAAAGTTCCCAGGCCAGGAATTGAACCTGCCACAGCAGTGACCTGAGCCACAGCACTGACAACTC
TGGATCCTTAACCCCCTGCACCAGCGCTATGGATCTTATACATCAAAATTATTGGACATGGATGTTAGTAGGCCGGT
AGCTGCAGCTCCGATTTGGACCCCTAGCCTGGGAACCTCCATATGCCTCGGATGCAGCCCTAAAAAACAAAACAAAA
CAAAACAAAAAAAAGAAGAATGCAATTCTGACATGTTTCAGCACAGATAAAGGTTGAAAACATTACGCTAAGTGAAA
TAAGCCAGACACAAAAGGACAAATAGTGTGTGATTTTACTGAGATCAAGCACCCAGAGTTGTCACATTCACCGAGAC
AGAAAGTAGAAGAGCGGTTACGGGGGTGGGGGGGATGGGGGTGGGCAGTGGGAAATTACTGCTTAAGCAGCACAGAG
CTTCTGTCTGGGATGATGGAAAAATTCAGATGGTTGACACTGGGGATGGCTGCCCAACGTGTGAATGTGCTTAGTGG
TACCGAACTATGCCCTCAAAAAGCATTAGAATGGTTTATGCTATGTATCTTTTACCACAATAAAAGGGGAAAAAAAA
GCCAGAACTAGGTGCATAGGTTATAGTGGTGAATACTATGCGACAAGCTTGTGGGCAGCGTGGTCACTTTATTCTTT
GCATTTCTCTGCATTTTTCAAACGTCCTATGATGAGCATACATTTCTTTTTAAAACCAGACAGAAGAGCGAGTTAAT
TAAACAAATCTCGTGGTTCTCTGACACTTTTGCCCAAATGCGTTACTGTCTTTTGCGTAAATGTAAGGTGTGTTCCC
TGTCCTTCGTTAATAAAAGGAGCCGAGCCCAAGGATGCCAACGAAAGGATACACCGAGGTGCTCAAGTCAACGACAG
GCACAGCGGCCCTCCTTTCTAAGACTCGTTGCTCTCGTCTATATTTAATAAGTTCCAAATAAAAACAGAACCCAAAC
AAATCCTCTAATGAACTTCCTAAGAAGCTGTCTGGCTTGGAAAAGCTCAAAGGCGAACTGAAGAGAAAGGGGGAACA
GCTGCTGTGTTTTTAGGGCATTAACTCACTGCAGCTGGGACAGTGCCTTTGTCAGTAGATTTCTATCCCTTCTTGCT
TCTGGGAAATGTTCTTGGGCAGAATGAATTCAGAAACCAGGAGAGGCTCCCCAGTGGTATTCCCTGCCAATCCATCT
GCTCCAGTACCCTCTCCCCACCCCAGAAACATGCTGAACAAAGATTTAAAGACTCTTGGTGTGAAGGGCAGCCACGT
GTCTGCCTGCCAGGGTGCCCTCCACCCCAGGCCGCCTGGGTCCACTTGCCCGGCTCCTGGGCCCTCTGCTCAGGGGT
GGCACAAGGGCAGAAGGTAGCTGCCACGATAAGCAGACCGGGGCTACCCCTGGAGTGGCCCCTCCCTGGCTACGTGA
CCTCTGCCTTTTTCAAATGTTCTATGATGAGCATACGTTTCTTTTTAAAACCAGACAGAAGAGCGAGTAATTAANNN
NNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNCGTATATGGACCATACCACCTTCCCCTGGCCCCAGGTTCTCACCTATGTGACTGAGG
GAGGTGGACTGGGGCACCTCTTAGATCTCTGCCAGCTCACACATCCTATGATTGCATCATCTCAAAAAGAAAAAGAA
AAACCAACAATACCTAAACCAAACTAAACCCTAAAACCAAAACCAAAAGCAGGGTGCCTTCTAGGAATCTAGGCCAG
GTTCTTACGTTTGGGGGGGCCTTGGGGTCCCTATCTACAAAATGAGGCACGGAGTTTCCACCATGGCACAGTGAAAA
TGAATTTGACTAGTAACCACGAGGACGCAAGTTCAATCCATGGCCTCGCTCAGTGGGTTAAGGATCTGGGGTTGCTG
TAAGCTGTGGTGTAGGTCGAAGACGAGGCTCGGATCTGGCGTTGCTGTGGCTGTGGTGTAGGCCAGTGCCTAGAGCT
CCAATTGGACCCCTAGCCTGGGAATTTCCACATGCCACGGGTGTGGCACTAAAAAGACCAAAAAAAAAAAAAAGGGA
AAAGAAAAAATTTGGCACAACCTTCCAGCTCGTTCCATGTCCAACATCTGTAATTCCTGAAAGGAAGGCCCCATCCT
CCCCTTGCCCTCCACCACGTCCTCTACCTCAGGCCAGGCTCACAAACAGGAAATATGACATTCGAGAGCAGCAGAAG
CACTGCTTGCTTCTCGACAGCATAGGGGCCGATGGAGAACAAAGAGTTTCTGAGCTTTTCCAGCAACAACCAGGGCT
CCATGCCCAAGACCTTCCCCAAGCAGTGCAGGCAGAGGACACTGCTGGGATGGGCTGGCCTCCCATGCCATCCCCGC
CCCGGTGTGTTCCCAGGGGCCCCCGGCAGCGCAGAATCAGCAGATAAGCTGTCTGGCCGTAATTACACGCTGATGCT
TGACCAAAGGTGGTAAAACCCTAAACAGGCGGAAGGCAGGGTGCAGGATTCCTGGACTCCAGTGCAGGAGTGGAGTG
ACCCTAGAGAGGCCCTACCCCTCTCTGGGCCTGAGTTTCCCCATCTATTTTTTTTTTTTTTTTTTTTTTTTTGTGTG
AGTGCGTGTGTGTGTGTGTGTATGTCCCCCTCTATTTGAATGAAAGGGCTAGAATGGGGCCTAATGGCAGCTCTTTG
CTTGCTCCGAGGTCTTCGGTTTTTCTTTTTTCATTCCATTTTTTTTTTTTTTTTTATGGCCACACCCACGGCATATG
GAAGTTCCCAGGCTAGGGGTTGAATTGGAGCTACAGCTGCCGGCCTACACCACAGCCACAGCAACACCAGACCCCAG
CTGCCAGATCCCTGACCATAGCGGATCCTTAACCTTACACCACAGCGGATTCTTAACCCACTGAGTGAGGCCAGGGA
TCAAACCCGCACCCTCATGGATCCTAGTCGGGTTTGTTACAGCTGAGCCACGACGGGAACGCCTGATGTCTTCTTTC
TGAAGGCAGTGTGTGGCCTTGATGAAAGGCCCCATCATCTTGCCTGTGTCTGCGTCCCAAATCTCTCCCTCACCACG
TGACCCTGAGAAACTGCTAAATCTTTCTGTGTTTCGTTTGCTCATTTGTAAAACTGGGGTTGCTGGGTGATGAAAAG
GCAGAGCTCCTGTAAAGCTCCTAGGACAGCTTCTGGAGTTAGCGCCCAGGAAGCGTGCGCTCTTGCTGTTTTATGAT
TTCTCTGGTTTCAGAATCGCTCCCCTTGCCCTGTTTGCCATCTGAAGAAGGAGCAAGCATGGCCCAGAGAGCCATAC
TGGCCCTGCAGTCCACGTCTAGCCCTCTCCCTCCAAGAAAGCACATGTGAATCTTGGTCAGCCAAGCACAGTGGGAA
GAGGGAACTATGGGAGAAAAGGCAGAAAATCCTACGATGCTGCCCCACAGCAGATGGGCTCGGGTGTCAGCTGCTCC
CAGGGGTTGCTGGGCACTAGAGAAGGCCTCCAGCTGCACCCAGAGTCAGTAGCGGAGGGAGGGTCCTGGGCTCATCT
CCAGCTTGATCCCCGAATGGGGAGGAGAATGACCCCGTGGGAAGGAGGGTGATGAGATGCAGAAGATGCAGCCGGGT
TTATCTCTGTTCCTACTTTGCCGGGACCATTCAGGGAAGAGGAGGCCACATTCAGTCATCTCAGCCCCGAGGGGAAC
AGGGAACAGAGAGGGGTGAGGATGACAGCACTGGTGGTCTCTCCCCTGGGGACATGGAGGTGTGGCCTCCCTCTGCC
ACAGGGAGGGTCCCAAACCTGCCTGTCCTCAGTGTTCTCACCTGCCAAGGGAGGAGACGCAAATGCCTGTTTCCACC
AGGCGCTCTAGGGTCTCAAATTGTGGCTGCGGACGGATGCATCGAGGAGGCACAGAAATTGAGAGTGTTTTACTAAA
GGACCAGTCCACAGGGGATTAGAAATAAAGGAAGAAAGGCCTGATCTTCTACCACACTGTCCTAGGACATAAAGCAT
GATGCGGGAGACAGGCAGGACCCCTGTTCCGCCTCCTGGGGCTACCCCGCTTGGCTCCAGTGAGCTCTGTGGTCCAG
GTGGAATTGTGGGCTCCCATCTGGCTGGGACGACTCACCCAGACAGACTGCCCTCCTGATCCGAGAGCATTTCACTC
GGCAGCAAATTCAACCCACCTCAAAATATCAGCTGCCCCTGATCAGGCAGGGCCTGGCTCCCTCTCTGCCAAGCCCC
ACAGGGCTGGGCTGGGATCAGTCATGGCAGCTCAAGGGAAGTCACGCTGCACCCAGAGGTAAAAGCTGTCCTGGCAG
AGAAAGAGAAAACTGATGGTCCTAAGAACAAGCACACTGGCTTTCACCCTTGAGGACGCTCAGTTGAGAATCTCGGT
TTGGGAGTTCCCATCGTGGTTGTAGATGGCTCTGGTGTAGGCCAGTGGCTACAGCTCCAATTAGACCCCTAGCCAGG
GAACCTCCATATGCCGTGAGTGCGGCCCTAAAAAGACAACAAAAAGAATCTCTGTTTGGCTGCCCTGTGTGGCAGGT
ATGCATTTATCAGGTATAGAGACATTTTACAGATGAAGGGAGCCCAGGGGATCTTTGCTCAAACTCTTTTTTTTTTT
TAGCTTTTTAGGGCCACACCCGTGGCATATGGAGGTTCCAAGGCTAGGAGTCGAATCAGAGTTTTAGCTGCTGCCCT
ATGCCACAGCCACAGCAATGCTAAATCCGAGCCACATCTGAGACCTACACCACAGCTCACGCCAAAGCTGGATCCTT
AACCCACTGGGCGATGCCAGGGATCAAACCTGCAACCTCAGGGTTCCTAGTGAGATTCATCTCCACTGAGCCACGAT
GGGAACTCCCAAACTCTTTTCTTTTACAGATAAAGAGGCTCAAGGAAAGGAGCACCTTGTCGCAGAAGCAGGATTTG
AACCCTCCAAGGCTCCTAGCCCCATCTGCATTCAGCCTGCCAATCCACGGTTAGGAGGGCCAACTGCACACATGCGC
AGTGTGGGATGTGGTGAGGAACCACACAGGAAAAGCCCTCAGTTCTCACAGAGCTCACATTCTAAACAAACAACAAA
ATCAGTCATTATAATTAACAAATCATTAAAGACATAATTTCAGGTGGGGGAGAGGGTTATAAAGCAAATTTAAAACC
TGGCGTGTTTGAGAGTGTTTTGGGGTGGGGGCAGCTGCTGTTTGGGAATGGCCTCTTTGCACTGGATCCTCTCAGGT
CCTCCCAAGCCAGTAGAATGCTGGAGCTGGCTCCTGCTGGCTTGCAAGGGCCACGTCTCATTAGGAATTTGGCGAGC
AAGTTGTTCACCACAGCCATTATTAAAAATTAAATTACATAAACTTAGAACTAAATGAATTATAGTACGACGGAAGG
TAATCATCAAAAGTCATCACTCCCTCGGGTTCCCAGGTGGCCTAGCAGTTAAGGGTTTGGTTTGTCCCTGCTGTGGC
TCAGGTTCGATCCCAGACCTGGGAACTTTCCAAGGCCACAGGCACGTGACCAAAAAGAAAAAGAAAAAAAAACTTCA
TTAATTTCCTCTTTGTATGACCACATACTATACTCTTGAAGTTGTTTATATCTATTGAATCTAGACGTAATAGATAC
TCCCAGTTCCTCCAGTAGTAGCTAGAAACTGGTCATGGTAGAAATATGTCTACTATGGAAACTGGCAAATACCCTCT
ACGAGGGCTTTCACTTTTCAAAGAGCTGGTGGTGAAATATTTACCAGCACAGCCTTCAGCTCTAATCCAGGCCTTCT
ATGCCTGTGGGAGTCTGGGTTCTTCCAAGGAGAGGGTGTGGTGGTATAGTCTAACTCTCCTGGGGCTGGGGGCGAGG
GGAGGTGGTGGGCAGTGCCTCCAGCCCTGTCCTCTTCTTCTTCTGTGTGTGTGTGTGTGTGTGTGTGTGTGTGTGTG
TGTGTGTGTGTGTGTGTGTGTGTGTGTGCTTTTCAGGGCTACTCCCTGGAAAGTTCTCAGGCTACATGTTAAATCGT
AGCTGCAGCTGCCGGCCTATACCACAGCTCATGACAACACTGGATCCTTAACCCACTGAGTGAGGCCAGGGGTCAAA
CCTGAGTCCTCATGGATACTAGTCGGGTTCCTTACTGCTAAGCCATAATGGGAACTCGGGCAGTCAGATTCTTAACC
CACTGCACCACAGCAGGGACCTTCTTCAAAAGTGTTTTTCAACAGGGATCTGTAAGAGGGTGATTCATTCCTTCCTT
TGTTATTTATTTTTGATAAATGAAATCCTATCATAAGCATACCAATATAAATTTAAAGGAACCCTGCCGAGAATCTC
TTTGTATAAATGCCTGCAGTCACTTCTGAGTTCCCCTAGATTTTCATAGGTGGAGGGACTTCCTTAGAGAATATAAC
TGTTCTCATTAACAGCAGACTGAAGTTACTATTACCTCTACTAATAACAATGACAACTGTAGCTGTCTTTTACTGGC
ACCACCTCAGGCACTAGGCACATATATTATCTCTAAAGTCTACATCAACCCATTTTACACATAAGAACGTTGAGGTT
CAAGGGTTCAATAACTTGACCTGAGGCCAGCCTGCTGCTCTGAAAGTTTCACAGAAGGCTTTTTCCTTCTGTAGCGA
CAGCCCTGCGACTCTCCTTAGACCTGCAGGATTCTGTGGTCCTACAGGACCCCCCATCTCTGGTGGTTTGGGAGAAT
TTCGTCACGTCTCAGCTTAGTGTAAGGAACTCCCTTCCATCAGCAGAACAGAATGAGCCAGACGCTCCCCCTGGACT
TTCTTTTTTTTTTTTTTTTTTTTGTCTTTTTGCTACGTCTTTGGGTCGCTCCCGAGGCATATGGAGGTTCCCAGGCT
AGGGGTCCAATTGGAGCTGTAGCCACTGGCCTACGCCAGAGCCATAGCAACGCAGGATCCGAGCCACGTCTGCGACC
TACACCACAGCTCACGGCAATGCCAGATCCTTAACCCACTGAGCAAAGCCAGGGATTGAACCCGCAACCTCATGGTT
CCTAGTTGGATTCGTTATCCGCTGAGCCACGATGGGAACTCCTCCCCCTGGACTTTCACCTGCAATGCAGGAAAGTG
ACCCAGGCCTGGTCACTTAGCAGCTTCCCACCCAAAAGAAGTAGCACTCAGGTTCTGATACCAGTGAAATGTTAACA
GCGGCTCCAGTGCCAGCAAGAGCTAGAATTAACTCCTGTTGGGAGACCCTAACTGTGTTAGGTCTGTTGCCTGACCT
CTCCTGGTTCTGAGCAGCTTGGTTTTCAAGCTCCCCCAGGAATACCATGAGCAACAACCAAAAAATCCTTCCAAGGC
ACATACCTCTTCTGCCTCGGTGAGCTAGAATCTCCATCGGTTGCTTGTAACCACAATTTCTGACCCGTACCTCATCT
CAAGCGCTTCTCAATATATCAGCCGCAAACATTCGCTGAGCCTTTCATGCCAGAGAAGGAGCTCCTAAGCACTCAAT
TAGTTTGCACAGAGGAATAGTAATCGTGCCTTTCTGTGCACAGCTCTGGCATAACCTATGAAAACGGAGTTTGCCAC
ACAAAATAGCAATCTGCAAACAACCACAGCTCAACTGAGAGCAAATCCAGGCCCAGTCCCTGCTCCCCGGGAGCCAT
ATTCCCCCTAAAGAAAACCCCTTCCTTGATTTTGTCAACGGTCTTGTCTTTCCCCACAGATGCCAGGCAAGTTCCTC
TTGGGGACAGCTGGCCGGCCACTTGAGGACTTGCGATTTCCCTGACGTAGGAGAAAGGACAGCTGGGTTTCTGCACA
CAGATGCTGCCAAGCCCAACGTCACCCTTCTGGGCAGCTGACCCATTGCCCCGGGCTTGCTCCCTCCCCTGTGCCCC
TCCAGACACCAGGGCCATCTGGATTCTGGAACAGCCATGGGGAAGATCAGGATGACTGGTTCTCAGGACCCCTTTCC
TTTGCCTGAAACGCTCTTCCTTTTTCACCCTCTACATCCTGCGGGCCTCAGTTTAAAGATCACTTCCTCAGGGAAGC
CCTCCCTGACCACTTCCCCAGACAAGTTCAGGGCCCCAGGACCCTGCCCTGTTTATCTCCTCCATGTCTCTGTCTGT
GCAGTTCATTGTTTACTGACTATCTCCCCAGCTGAATTCTAGCCTCTGCACAGGAAGGGATTGCACCTCTGTTCACC
GAATCTCAGGTTATCTAGCACAGCATGTAGTTCCATAAATCCTGAACGCTTTAAAGATGAGTGAAGGACATTCTGGC
GGCTCAGTGAGCGCTGAATGAGTATCTGATTTAAAGCATGCATCTCAGCAACAGGTGCATCTTTTAGGACCACCGTT
TTCTGGTGCCCAAACTCACAAGGGCAGGGTGAAAATTTAGCCATCCCTACTTCTCCCCGGGTCGTTTTTAGTTTGAA
GGTTTGTTTCCTGTGGGTTGGGACTGGCCCGATTTTTGTTTAACAGCAGCTATTGCTCAGAGAGGAGTTTGCTAGAT
GCCAGCCTTATACCACCTGGTTGATGGGGAAACTGAGGCCCCTACCACTGGCTGCACCAGCACCGGCGGGGCGAGAC
CAGCTCTCTTTCAGCCCAGAGCTCATTTCAGGGTCCTTCGCCCCACATGGGGCCAAGTCCAGGGCATGCGAAGCAAG
GCTCGGGAAGATAAGGGCACCCAGACGGGGATGGAGTTTGAAACTTTTATTAAGAACGAATCAAGAGGGAATTCCCT
TCATGGCTCAGTGGTTAACGAACCCGACTAGGATCCATAAGGACAAGGGTTTGATCCCTGGCCTCGCTCAGTGGGTT
AAGGATCCAGCATTGCCGTGTAGGTCACAGAGGCGGCTCCCATCTGTGTTGCTGTGGTGTTGCTGTGGCTGAGATGT
AGTCTGACAGCTACAGCTCCGATTCGACCCCTACCCGGGGAACTTCCACATGCCATGGGTGCAGCCCTAAAAAGCAG
AAGAAAAAAAGAAGAAGAAATCAAGAGACCTGGCCTCTCTCTCTGCCCAGCCTCTTCCAGCTGCTACCTTCCACTCT
CTCCGGCTAGTTTCAGGTTGAGCAAGGCCAGGCAGGAGCCCTCTCGGGGGCTGAGCATGGATCTGGGCCCCAGCAGC
GCCCCCAACCTTCAGATTCACCTTCACTCTCCTTGCTCAGGGCCCACCAGGGTCTCCAAGCCAAACTATGTTTGAAG
TCAAGACCAGGCTTTCATGCTTTGGTTCTGCCACTTCACTCTTGAGAGATGGTGGCCAAACAATTAAAACGCTGAGC
CTCAATTTCCCTGCCTGTAAAGTGAGGAGGCGGGGGGATAATTCCTGCTTTGCTGACTTCATAGGGCTTTTGTGAGG
CTCAGGCGAGGTAGATATATGTACTCACTCGTCTAACTGTCCACTAGCTTAGAGAACTCTAACAACAACTCTAGGAG
TTCTGGCAGTGGGTTGAGAATCCGACTGCAGCTGCTCAGGTCACTACAGTGGCACGAGTTCGATCCCTGGCCCTGTG
CAGTGGGCTAAAGATCTAGATAGAGTTGCGGCAGTGATGGCATAGGTTGCAGCTGTGGCTTGGATTCAATCCCTGGC
CCGAGAACTTCCATATGACGTGGTGCAGCCGTAAGGGAAAAAAAAAAAAAAAAAAAAGATACTGTTTTTCTGGTCCC
ATTAGGGTCTTGCGATCAACGTGTAGCCAGCCCATGTCCTCCAGGGCCCAATCCTCCACCCAACCTCTCAGCCAGGC
TCTCCTCTTGACCACATCCTTCTAGAAATCCTTTCTGCCTCTGCCTTCCTGGATGTGCTCCCTCTGGGCTCTCCTCC
ATCTCAGGTCACTCATTCTCCCAGTTAGGACCTGGCCCACCTGGCAGCTCCGTGCTTTTTCCTGCCATTCACGTCAG
CCAACCACACAGGGCCTGGGACAGGAACTGCAGGGAACACATACCAACACTCAGATCCCTGGATAAGGCTTGCGTGC
GCATTCCCTGGGGCACAAAACATGCGCACAAAGCATTGTGTCCCCACCCCACTGCCCTCACCACCCCTCCTTTGCTG
GGGCATAGGGCAGAACCCACAGCAGACGGAAATTCCCAGGCTAGGGGTCTAATTGGAGCTACAACTGCCGGCCTACA
TCACAGCCACAGCAACGCCAGATCCAAGCCACATCCACGAAGTACAGCACAGCTCACAGCAACGCCGGATCCTTAAC
CCACTGCGCGAGGCCAGGGATTGAACCAGCAACCTCATGGATACTTGTCAGATTCATTTCCACTGTACCCCGACAGG
AACTCCACCACTCCTCCTTTAAGAGACTCTATTTGGCAATAAAGCCAGAGCCAAGGCTCTGGCAAGAGTTGCAGCCA
GGTCTGATCATAGGCAGCCAAGGTCTGTGGCCCTCCAAGCCGGGCTGGGACAAGCCAAGCAGATCAGCTCCTCGGCT
GGAGATTTCAATGACATATTTTTAGGTCAGCCTCTCTTTAGAATTGCAAGGACTTTTATAAATAATTCTGGGTTAAG
TATATTCCACATGATGACCCTTCTGCCTTCAGTCCACAGTCCAAATCTACATCACTCTCTGGTGTCCCAGACTGACC
CACCTGGCTTCCCTCTCTCAAGACTAAGGCTGAAGCTTTTATCAGCAGACCTTGCAGCCCAGGGCAGGGGGTTGGGC
AGGGGGGAAACGACTTTGCCCCAGTTGCCCTTGGGAGGCCACTTACCCACAAGTGTGGGTTAAGTAAAGGGCACTGC
GGTCACATGCCCAGTGTGCCATCTGGCTTCAGCAGCCACCGTCAAAGAGGGAAGAAAAAGTGACATGCAACAGAATG
TAACCGGGGCATGGCCTGCAGGATGCCCAGGGACCTGGGGGGCAGAGGGGTGCCAAATTCATGGGGGGCTTCTCAGA
GAGGGTGGTGATTAAGATGGGCCTTGAAGGATGTGTAGGAGTCTGTGGGAGGGTTTGGGGAGGAGGTGGGAGGGTGT
CCTGGGCATGGGGAAAAGTCCAGAGCCATCGAACCAGGAGAGGGTTTCAGGAATTGCAGCAGTTCCCTCAGGCTGGA
GCAGAAGTTCCAAAGGATGGAGTGGTGAGGGTGGTGAGGGCTTCAGAGGGCTGTCTGTATGGGACCTTGGAGGTCAC
CCAAAGGAATGTGTGCTTTATCCTGAGAGCAGAGGGAGCCTTGGAAAAGATGGAAAACTCCAATCAATTAGGTGTTT
GGAAATGAGACTTAGGCTGCAGGGAGAGGGTGTATAGGAACAAAGAACAGGGAGCATGCAGCAGCAGGGGCTGGGCT
GAAGAGGGCTGCCCACCAGCACAGCAGGGGCAGGGGGGCTGGAAGGAAAGGGTCTCTTTTTTTTTAGGGCCACACCT
GCGGCATATGGAGGTTCCCAGGCTAGGGGTCGACTTGGAGCTGTAGCCACTAGTCTACACCACAGCCATAGCAATGC
CAGATCCTTAACCCCCTGAGCAAGGCCAGGGATCGAACTCATGTCCTCATGGATGTTAATTGGGTTTGTTAACTGCT
GAGCCATGACAGGAACTCCTAAAGGGACACTTTGGAGAGCTGGTAAAGGGGTGGGATTGACTGAACTAGATTAGACT
GGAGGGGAATGTTTGTTATGCAGCATAACTGCAGCCAAAGCTAACAGAGGGGCCACATGAGCAAATATATAGAGACA
GAAAGGCCACTGCCATGCTTGAAGAAGCGGAACGATGGTGCTGATGGTACCAAAGAGCAGGCTGTGTGATGGGCATT
AGTTTGGAGAGAGAAAGATAGGTGGGGACCTGCACGAGGGAGTTTCTAACAAATATATGAAGTTGATTGGATTGTTG
TTCCCAAGTATCTATTCTGGGCCAATAGGCAGAGCTTATCGCAGTCCCATTGACTTTAGACTCAGTCACATGACCAG
CTTTGACCAATGGAATATGGATAGAAGTGACCATGTGCCAATTCAGAGATTTAATTTTTTTTTTTTTTTTTTTTGTC
TTTTGTCTTTTGTTGTTGTTGTTGTTGCTATTTCTTGGGCTGCTCCCGCGGCATATGGAGGTTCCCAGGCTAGGGGT
TGAATCGGAGCTGTAGCCACCGGCCTACGCCAGACCCACAGCAACGCGGGATCCGAGCCGCGTCTGCAACCTACATA
CACCACAGCTCACGGCAACGCTGGATCGTTAACCCACTGAGCAAGGGCAGGGACCGAACCCGCAACCTCATGGTTCC
TAGTCAGATTCGTTAACCACTGCGCCACGACGGGAATTCCTTATTTTTTTTATTTTTTTGTCTTTTTGTCTTTTTAG
GGTCTCACCCACGGCATATGGAGGTTCCCAGGCTAGGGGTCCAATCAGAACTGCAGCCGCCAGCCTATACTAGAGGC
ACAGTGGATCCAAGCTGCATCTGTGACACTGGATCGTCAACCCACTGAGCAAGGCCAGGGATCGAACCTGCAAACTC
ATAGTTCCTGATCAGACTCGTTTCCACTGTGCCACAACAGGAACTCCCTCAGAGATTTTATGTTATTTATTTATTTA
TTTATTTGGTCATGTAGCAGTTTGATGTGGGATCTCAGTTGCCAGAACAGGGATTGAACCTGGGCTGCATCAGTGAA
AGCACCCCAAGTCCCAACCACTAGACTACCAGGGAACTCTCAGAAACTTTAAGAAGCATTGAATTATCTCTTTCTTC
CTCCAGCTCTCAGCATCAAAATGACACATTCTAGGTAGAAGGAGCAGCTTCAGCCTGGGTCCTGGGAGGAGAAGATA
CATGCTGCAGATATTCTATCCTGCTGCCACCTGGAGCAGATCTACAAAACCATGCAGTTGCAACTGCCTTCTGGCTG
ACAAGCAGTGTGAGCAATAAATAAACCTTTGTGGTCGTAAACTAAGATGGGGGGGATGTTTGTTATGCAGCATAAGC
TAACTGATACACACTATATATGTGAGATGATAAGGATGCAGATGGTGAAGAACATCACATGTCACGATTAGTTGTTG
TACACATGGTGAGTCAACAAAGAATTTTGTAATTGATGAACCTTCTCCACCTTTCCTTTAAAGCCAACCCTCTCCAC
TCCCTTCTGCTCCTCCTAGCCCCTTGCTCTATCAGCCACCCCTTCCCTCGCATGGACTGAATCCTTCCCCTGAAACT
ATATCTCACTTGTCTCTTCCATCCTAAAATCCTTTTCTTTACTCTGTCTTCCTCCAACTCTAGCTCAGTCTCTTCCT
CGACCATCTCAAACAAACTTCTTCTTCTTCTTTTTTTTTTTTTTTTGTCTTTTTAGGGCCACACTTATGGCATATAG
AGGTTCCCAGTGTGTGACCTACACCACAGCTCATGGCAACGCCGGATGCTTAAGCCACTGAGCAAGACCAGGGATCC
AACCCATGTCCTCATGGATGCTAGTTGGGTTTGTTAACCACTGAGCCACAATGGGAACTTCTTCAAACAAACTTCTT
AAACGAGTTGATTCTCCTCATTATCTCCACTTCTTTCTCCCTCACCTCCAAGCAATCTAGTTTACCTTCCCTCCACC
CCACCAAAACCATTCCCAGTATATTTCAGCAATCTAATAGTCCAGTGCAATCCAGTCCTTATCTTCCTAGACTGTTC
CACATCATTTAGCTTGGAACTAAATTCATTTTCTCCCTGCCCAACCTCAAATATTCTTCTTTCCATGGAGTTCCTGT
CATGGCTTGGTGGTAACAAACACGACTAGTATTCTTAAGGACTCCGGTTCCATCCCTGGCCTCGATCAGTGGGTTAA
GGATCCGGCATTGCTGTGAGCTGTGGTGTAGGTTACAGACTCGGCTCAGATCCCTCGTTGCTGTGGCTCTGGTGTAG
GCTGGCAGCTGCAGCTCCAGTAAGACCCCCAGCCTGGGAACGTCCATATGCCACAGTTGCGGCCCTAAAAAGAAAAA
GAAAAAAAAAATTCCTCTTTCCATATTCTCTCAGCTAGTGGCACCATCATTCATCCAGTGACTCATGACAGAAAGCC
AGCATGACACAGTGAATTCTGCTCTGTAGTTGTCCAGTCTGCGGTGCCTTTGAGACATCCAAGAGGAGATGTCCCAA
GGGCAGCAGCTAAACATGTGAATTGGGGGCTGACAACAGAGATCTGAAGTGGAGATACCGATGACTGTTAGAGGCAG
CATTTAAAGCCATGTGCATGCGTCAACTTGTCTATTTATAAAGTACAAGGACCTGGTGATACATAGAGCGCTCTCCT
GAGCCTATACATTCCCCCTCCTAAGACCACAATTCCAGGTACCACTTAGTTCCTTCCTTCCCAAGTCACGGCTCACA
GGGGCCTCCATATCACCACCTTATTTCATATTCTCCCCCCCCAACATGTTGCCTTCTCCAACAACTCTTAAAATTCA
TAAAAACAGAAGATATAAGATACCACTACCCAGGCACTAAAATGCCTAAAAAACAAAACAAAACGCACCAATGTGCT
ATCACTCACATGTGGAATCTTTTTTTTTTTTTTGGCTTTATTTAGGGCTGCACCCAGGCGGCATATGGAGGAGGTTC
CCAGGTTAGGGGTCTAATCAGAGCTGCAGCTGCCGGCCTACACCACGGCCACAGCATCATCAGATCTGAGCCGCATC
TGTGACCTACCCCACAGCTCACGGCAACGCCAGATCCTTAACCCACTGAGCGAGGCCAGGGATCGAACCCGCATCCT
CATGGATCCTAGTCGGATTCCTTTCCACTGCGCCATGACGGGAACCCCCGCATGTGGAATCTTTAAAAAAAAGGACA
CAATGAACTTCTTTACAGAACAGAAACTGACTCACAGACTTTGAAAAACTTTCAGTTTCCAAGGGAGACAGGTTGGG
GGTGGCGGGGTGGGTGAGGGTTTGGGATAGAGATACTATAAAATTGGGTTGTGATGATTGTTGTACAAATATAAATG
TAATAAAATTCATTGAGTTAAAAAAAAATGAACAGGAGTTCCCTTCATGGCTCAGTGATTAACAAACACGACTAGGA
TCTATGAGGATGCAGGTTCAATCCCTGGCCTTGCTCAGTGTATTAAGGATCTGGCGCTGTGGTGTAGGTCGCACACA
GAACTCGGATCCTGCGTGGCTGTGGCTGTGGCGCAGGCTGGCAGCTGTAGCTCTGACTGGACCCCTAGCCTGGGAAC
CTCTACATGCCGTGGGTGAGGCAAAAAATTAAAAAAAAAAAAGAATTAATTATAAAATAAATAAATAAATGAACAAA
TGTAGATGTTAAACACTTATCATGGAACACTCCTGGAAATAAAAGAAGATTAGAACTAAAAAAAAAAAATGGACAAT
ACGCAAACACTGTCGAGGATGTGGAATAATCGTGTTTTATACATTGCTGGGGAATCTAAAACGGTACACCCTATGAC
CCAACAATTTCAATCCTAGGTGATAACAAAGGTCCACAAAAGACTTCTACAAGAAATAATAGCCCAACTTAGAAATA
ACCCAAAGGTTCATCGAGACGAGAATAAATATGCAAATGATGGTATAGCCTTAGAATAGAATACTACTCAGCACTAA
AAAGAAAGACACAGATGAATTTCACAACATACACAACAACACAGGTGAGCTTCACAAACTATATATATATTACATGG
AGGGAAATAAGCCAGATACACAAGAGAAATACAGTGTGATTCCATTTATGTGAAGTCCAAGAGCAGGCAAAATTAAT
CAATGTTGAATAAAGTGAGAAAATGGTTGCTTGGAAGAGGCGAAGGAAAATTGATAGGAAATGGGAACTTTCCTAGG
ATGACGCAAAGATTTCATATCTTATTTCGGGTGGCCACTTCAAAGGTGCAAACAACAGCTAAAACTTGTGGAACCCA
ACCCTCACCACCTGCGTATTTTATTGTTTGGAAATTATACTTCAGTTAAAACATTAGGAAAAGAAAATAATTTTGTG
AAGTATCAATAAAATAACGAAAATGAAGAGACTCTAAAGGGCAAAAACACATTCAGTTCAAATATATAAATTATATT
TGTGCTATGTATGCATCTATACGAATGTCCAGCCCCCCTTAATGTAGCCCCCTTTCAGCCATTCTCCGCTCACCCTT
GCCCCCATCCTGATGGCCTCTGTCCATAGCCATTTTCTAGCTGTCATCAGAAATGATGCAGTGAAAGAGCAAAAGCC
TTAGAGCCAGATAGAGCTGCATTTAAATTCCAGCTGCTGAGCACCCATAATCGAGTTACTCGGCCTCTCTGAACGTT
CATTTCCTCAACTACAAAATGGGTTGATGAGACACAATCAACCCTGTTGGGCTGGACTAAGAGAGAGGCAGTGTGCT
GATTAGTTTCTGGGAAACCTAATTCTTTTGACCTCAGCCTGTGAAACCAACTTGGTTGTGCAAGGCCCACTGCCGGC
CTGGAAAAGCCCAGAGGATGAGACTCACGGGCTACTTCTCCCTGAAGGATAGGGAGGTGGTCCTGGGAACCCAGAGT
CTTTGTGGGCTGGTGCTAAGAGTCGAGTCGCTAACTCAGAGCCATCAGGGCCAGGAAAACCTATGACCTATGACAAA
GGAGACAAGTTTCCTGCCAAGGGTTGGCCACCTCAGGATCTTGCCCAAATCACTTTGCACACCCCTAGATTCCATTT
ATCCACCAAAAATGGCCAGAGGAGCCTGGATCTGAAGAATTTGATACTAAAAACAGCTTCTGGAATTCCCATAGTGG
CTCAGCAGAAACGAATCCGACTAGGAACCATGAGGTTGGGGGTTCGACCCCTGACCTCGCTCAGTGGGCTAAGGATC
CAGTGTGGCTGTGAGCTGTGGTGTAGGTCGCAGATGCAGTTTGGATCTGGCGTTGCTGTGGCTGTGGTGTAGGCCAG
AGGCTACAGCTCCGATTAGACCCCTAGCCTGGGAACCTCCATATGCCTCGTGTGTGGCCCTAAAAAGTCAAGAGTTA
AAAAAAAAAAAAGAGTTAAAAACAGCTACTATGTCTTGGGAGCATTGCGATGCAAGTTTGTTCTCAGCCAGGCACAG
GGTTAAGGGTCTGGCATTGCCACAGCTGCGGCTTCGGTGGCAACTACAGCTCGGATCTGATCCCTGGCCTGCTCCAT
GTGCTGCGGAGTGGTCAAAAAAAAAAAAAAAAAAAAAAAAAAAACCCAAACAAATAGCCTCTGGTGTTTCCCAATCT
ATAGAAGAGATCAAGGCAGGACCAAACTGGTTCTGTCCGAAAGAAGGAACGGAAGAGTCAGAGTCGGAGCCCTGCCG
GCTAGCTCCCCTCCTCCACCTTGGCGTTTCCTGAGCCAGGATCCTAGGTCTCCCAGGGGCAAAGTTTGAAATCTCCC
TGACCAGGTAAACCCTAGGGCCTCTTTTAGCTCAGTCTTATCCAGTCGTGGTGCATCTGTCAAGTGTAATAATAAAG
AGGATCTGCACCTGCCCCCCCACCCCATCTGGTAGGGGAGGCAAGGTGCACCCAGAAATAACTCCGAGCAAGGTACA
AAGTGCTTAGTGTAGCCAAAGAAGCACATAAGTCCAATAAAGCATCCACATTCCCCCCCCACCACACACACACACAC
ACAACCTCTTCGCACTTGGCATTTCCTTACTTCCAGCAGTCTCTCTATTTCAGGTTTGTGGAAACGGGTTCTCCCTG
GAAAAGGGTTTCCCAGCTAGGAGGCGGCCCGGCCCCGACTCCCCCTCTCCCCCACCACCCCCGGTCCCCGCACGTCC
AGCGCTCCGAGACCCACCCATTTCCAAGCACAAGAACAAGGCGACAAGGCCCGCTCAGGGGCCAAGAGGAGGGCAAA
CGACGACAAGCAAAGCCACAAAAGCAACCGTCCGGGTCTCTTGTCTTTCCTGGGGGGAGGAGCGGCGCCCGCAGACG
GTCTCCGCGCCTCCCTCCCTCCCGGGCCAGCGGGAAGATAGGGGAATCTCAAGTCGCTCTGCTTTCTCTCTTCGCGC
ACTGACATTTTCCCCCACTTTACTGTTTCTTGGACGCCTTTCAAGAGTTTGTGCAACCAGTCTGTTTAGCTGCTTTT
CTGCCATTTTCAAACGCGGGGTGGTGTCCCTTTCGAGTGGGAACGTGGTGGCTTAAAGTCTGGAAGGGACCCCTTCG
CCTCCCGTCACCCCGCTGCAGCGGGCCTCTTCGCCGCCAAAGTTTCGGCGTTCCAAAGTTTCCCCCGGCCGGGTTTC
GGGCTCGGTCCTCCGCTCTCTGAGCTCCCCGACTTCTCCCTCTCTGTGCGCTCAGGGGTTTCTGTGCCCCTCACTTC
ACTCTCAGGTTCCCTCTTGCGGAGGCATCCTCTTCCCACCTAGTCCCGGGCGAGGGAGGCCTCCGCCTCCCCTGCCC
CACATTGGGAGACAGACCCCTCCCTCCTTTCGAGACTTCCCGGGCAGTCCTCCTCCTCTGCGCGCCCCGAGCCTCCC
CTCTCCCGCCTCCATCCGGCGGACCCCGTGGAAGCCCGCAGCCCCTCAGGCCCGACAAGATGGGGACAGAGACGGGG
TCAGAGTTGAGCACAGAGGTAACGACGAGAACAAAAGCGGGGACACGGCAGGGCAGCAACAGGGCAGGGCCGGCGCG
GTGGCCTGTCCTCTCCCCGCGCTGCCTCCACGGCGCCCGCAGCCCCGGGCCGGGCGGGACTCGCGGCCTCCAGGGGC
TCGGGCAGCGCCCAGCGGGACCCACCTGATCGGCAGAAGCTGGGTGCGCTCGGGGATGGCCCACACCTCGGCTCCCG
GCCCCCCGGCGGCGTCCTCGGCTGAGGGAACAGTGGCGCGCGGCGTGCTCCTGAGCTCGGCAGGGCGTGCCGGGGCG
GGGTGTGCCGCCTGCGCTCCGGCCCGCCGGCCGCTGTGTGCTCCTCCGGGGTGGCGGGCAGGGGCGCGAGGAAGCCG
GCGGGCACTGGGCGGCGGGCGGCGAGCTCCCCGCTCCACCCGGCCCGCGGCTGTTTGTGCAGAGCGGGTCCCGCCCC
AGACACGGCCGCTAGGAGGCCGAGGGCGCGAGTGCGCGAGTGCCGGTGCGCGTGTGTGTCTGGTGGCCGGGAGGCGC
AGGGGGTGTTTGTTTCATTTTCACTCAGGCAGAAAAAAGCCTGAAACCAGCAAAAAAAGAAAAGAAATTCCCTGGTG
AGGGTGGCTGGGCCTCTTTGCCTTCTCCGGCCTGCACGTGGTGGGGGTGGAGGGACCCGGAGGGTGGGGTGGGGTCT
ATCACCCAGTACTGCAGGGAGGGGCCCCGGAG

| | |
|---|---|
| SEQ ID NO: 12 | GGTA1 cDNA Sequence |

ATGAATGTCAAAGGAAGAGTGGTTCTGTCAATGCTGCTTGTCTCAACTGTAATGGTTGTGTTTTGGGAATACATCAA
CAGCCCAGAAGGTTCTTTGTTCTGGATATACCAGTCAAAAAACCCAGAAGTTGGCAGCAGTGCTCAGAGGGGCTGGT
GGTTTCCGAGCTGGTTTAACAATGGGACTCACAGTTACCACGAAGAAGAAGACGCTATAGGCAACGAAAAGGAACAA
AGAAAAGAAGACAACAGAGGAGAGCTTCCGCTAGTGGACTGGTTTAATCCTGAGAAACGCCCAGAGGTCGTGACCAT
AACCAGATGGAAGGCTCCAGTGGTATGGGAAGGCACTTACAACAGAGCCGTCTTAGATAATTATTATGCCAAACAGA
AAATTACCGTGGGCTTGACGGTTTTTGCTGTCGGAAGATACATTGAGCATTACTTGGAGGAGTTCTTAATATCTGCA
AATACATACTTCATGGTTGGCCACAAAGTCATCTTTTACATCATGGTGGATGATATCTCCAGGATGCCTTTGATAGA
GCTGGGTCCTCTGCGTTCCTTTAAAGTGTTTGAGATCAAGTCCGAGAAGAGGTGGCAAGACATCAGCATGATGCGCA
TGAAGACCATCGGGGAGCACATCCTGGCCCACATCCAGCACGAGGTGGACTTCCTCTTCTGCATGGACGTGGATCAG
GTCTTCCAAAACAACTTTGGGGTGGAGACCCTGGGCCAGTCGGTGGCTCAGCTACAGGCCTGGTGGTACAAGGCACA
TCCTGACGAGTTCACCTACGAGAGGCGGAAGGAGTCCGCAGCCTACATTCCGTTTGGCCAGGGGGATTTTTATTACC
ACGCAGCCATTTTTGGGGGAACACCCACTCAGGTTCTAAACATCACTCAGGAGTGCTTCAAGGGAATCCTCCAGGAC
AAGGAAAATGACATAGAAGCCGAGTGGCATGATGAAAGCCATCTAAACAAGTATTTCCTTCTCAACAAACCCACTAA
AATCTTATCCCCAGAATACTGCTGGGATTATCATATAGGCATGTCTGTGGATATTAGGATTGTCAAGATAGCTTGGC
AGAAAAAAGAGTATAATTTGGTTAGAAATAACATCTGA

| | |
|---|---|
| SEQ ID NO: 13 | GGTA1 Protein Sequence |

MNVKGRVVLSMLLVSTVMVVFWEYINSPEGSLFWIYQSKNPEVGSSAQRGWWFPSWFNNGTHSYHEEEDAIGNEKEQ
RKEDNRGELPLVDWFNPEKRPEVVTITRWKAPVVWEGTYNRAVLDNYYAKQKITVGLTVFAVGRYIEHYLEEFLISA
NTYFMVGHKVIFYIMVDDISRMPLIELGPLRSFKVFEIKSEKRWQDISMMRMKTIGEHILAHIQHEVDFLFCMDVDQ
VFQNNFGVETLGQSVAQLQAWWYKAHPDEFTYERRKESAAYIPFGQGDFYYHAAIFGGTPTQVLNITQECFKGILQD
KENDIEAEWHDESHLNKYFLLNKPTKILSPEYCWDYHIGMSVDIRIVKIAWQKKEYNLVRNNI

| | |
|---|---|
| SEQ ID NO: 14 | CMAH Genomic Sequence |

CTACCCAGAGCACATCAGGAAGGACTTCCAGTCAGGTGGTGTGAGGGGGAGTTTTATTTGAAAATGATTCCAAAACC
TGTAAGAGATAAAGTAGAAAAACATGTTTTGGAAACTTCCATGCCTGCTGTATTTGCCAAAATCTGTTCAGTACCTG
GTACTCAGCTTTCCCTGAAAGATAGCGTTTCTGTACTGTTTCAGATGTTCATTTAACTTAGCATTTTTGATACAGAA
TGCAGTCCTTAAACATGACAATTGTGTCTTCCTTCTATTTTTCTGTGACATGCCTTGCTTTAAGGAATTCTTGTATG
TAAAAATATAGAATCTGTACACAAAAACATTAGGACCTAGTATTGGTGAGAGGGCAAGTAAATGGGTTATATGTTAT
TTCTGAGAAGGCGAGTTGGCTTCCTGAAGATCAGTCTGGCAGAGTATAGATTATTCTAAGAAATCATTATGAATTTA
TCCTAAGAAATTTATCCTAAGAAATCATTATGAAAGTGTGCAAGACACACCTACATATTTCTTTGCCAAAACATCAT
TTCAAATAATGAAAAGTTAGAAACTTACAGGGTAGATCAAAGACTGTTCAGTAATCATGCAGGTGTACAGACGTATG
TATAGTATTATCCCATTTTCATTTTTTGAAAAAGTGCTTGTGGTATATGTGCTTGTAAACAGAAAAAGAAAGATGAA
CTAGACACCAAAGTACAAATTGCTCTCTGGATGGTGGGATCATTTGTGGTTTAACTGTTTTTTGAATTTAAAAGTTT
TTTTTTTTCCAAATTTTCTGCGGTAGATCTGTGTTATTTTTATGATCAGAAAAATATTTAGTAAACTAAATCTCATT
TTAAAAGCAACAAAGATATATTGGGCTATGACTGCTTCCCAAGATTCATCACAGGATCCTTTCACATTTATGAACTT
TGCTATCAAAACAGTATATAGAAAAATAGTCTTCAGAATCAATAGCCCAGAAGTTTCCAAGATGTAATTTTTTTTAA
AAGAAAAGTTATCTTTGAATCTTTCTCACTCAAATTTGCTCCATTTCCTTTTTTCCAGAACAGAAGTCAGCTACGAA
CTCTGTTGAAAATGAACAAAATGTTTTCATTTTGCTTTACAAATGAAATGGTTTCCAAATGGAATGTTTTACAGACA
TTAAAATAGTTGAGGTTGGAGTTCCCATCATGACTCAGTGGTTAATGAACATGACTAGGATCCATGAGGATGTGTGT
TCGATCCCTGGCCTCGTTCAGTGGTTAAGGATCCGGTGTTGCCATGAGCTGTGCTTGTAGGTCACAGACACGGCTTG
GATCTGACGTTGCTATGGCTATGACGTAGGCTGGTGGCTACAGCTCTGATTAGACTCCTAGCTTGGGAACGTCCATA
TGCTGCAGGTGTGGCCCTAGAAAGACAAAAAGACAAAAAAAAAAACCCAAAAACTGAGGTTGACCTGTGTGTCCCAA
CACTAGAAATACCAAAGATATTAATGAATAAAAAATGCAAATTACAGATGTACCAGGATTACATTAAAAAAAAAAAC
AAAACAAAACCCAGGAATGATAACCTCCCCTCCTCAACTATAAGGGATGTTTTATTGAGAAAAAATACATTTCTTGA
AATGCTGATATGCTCAAAAATAGGCCTGGGGTGATACAACTATGCTGTTACCAAGTGTTACCCTGGAGAGTGGGTGG
AGAAAGGCAGGAAACAGGGTTTTGTGGGAGGTGTGGGGTTATTTCCTTTTTATTTTATATAATTCTACATTCTTTAA
ATATTTTTAAAGCAATTTCAAGATATTCAAAAAGAAATCTATAAAGAAGAAATGTCAAGACAGGCCTGTGCGTGCAA
GCTCATGGCAGAAGCGGGGTAGGAGGCTTGCCTGCTTCAGACTAAATTCCTGACCTTTTCAGAGGGTCAGTGGTCAT
GAAAGAATGCATTCTCCCCTCTTGCTGATTATTTTGCAAATACAAAAATGGCAAATGGGGCTTTCCAGCATTTCAGC
ACAAATATTCCAACTAAAGCCCTAAGGACCTATACGGTTTTGCTATGAGAAACTTACGTGGTTTTTGAAGCTCAACC
AGGGAGAAACTTGGAGGATCATCCCCTTAACCAACTAGTTCACCAAATTCATGCTCAGAGTTGGGCAACATGGGAGA
TGAATGTCTTCCAGGATCACAACTTTGCCATATCACCCCATCCTCATTCTTGTCATAGTGATTCTTAGTAATTTTGC
AGTGTCTTCAGATAAATTCTGAGGAGTGGAGCTGCTGGATCCAAACACACCCTCTCCCTTTCATAATGTCCTTCCCT
TCCCTGTACTCTAAACTACTTGTATACAGGATTGAAGCACATGGGCATGAATGTCCAAATGGTGACTCTTTGAAAGT
TATCTTCCTAACCAGATTTGCCTTTCAAGGTTAACAAAGAAAAAAGCTCTAACGGTGGAATCTCCATGGCCATCAAC
ACTGCAGGGCACAGTCAGTCACTGACTCTGCTTATATAGCCCTGGCCTCCTCTGCAGCAGCCTAGGGCACACACGAC
AGGCATTTTCGGACTTACAGATGATGGTATATATCAGGATCCCGCTGAAGCCGGGTTTGGAATCCTATGTACAAGTC
ATCCCAGAGCAGACCATTCTTTACCACGTGTCTGATGACATCAACCCGGCTCCGAATCTGAAACAGAGGAGGAATCA
CGAGTTAGGCGCAACCCAGCCAGTAGAGAGTGTCAGTATGGACCCCTCGTGTCCCGGAGAGAAGCAGCTGCCTGTAA
GGGCAGGGATGGAGGAATCAAGGAGAAAAGCCTACTGAAGCAGATCTCACAGGCCGAGGGGGAGAGGGGCCCCTGAG
TGCAGCAGAAATCGAGGGATGGAAACAGGAAGTGGATCAGGAGCTGGGGGTGCAGAGTGGCAGAGAGTACAGACAGA
GTTGGATGGCTGGGTATGAACCCCCAATATAGCTGTGTGACCTTGGCAACCATTCTGTGCCTCAAGTTCCTCATCTA
TACAGTGGAGGTAATAGAACATTCCTCCTGGGGCTGTTGTGAGGATTACCTGAGCCAGTGTACTTAAAATACTGAAA
ACAAGGCCTGCCACAGAGCAAGATTACCTTAATTCGGTGGTCAAGGCCCTTACCTTCAAAGAATCCCAACTCCTGAC
ACAGGATCTGTTGAATAGTCAGAGGTGCACAGGGTTAGGAGACAAGCAGAGATGGTTTTGAGTTTCAGCCCAGCACT
TACTAATCATGTGACCTTAACCTTGCTAAGCCTCGGTCTCCTCTGTGACTGTTGTGAGAAAAAAAAAAAGAGATAAT
TCATAAAAAAAAAAAAAAAAAAGAGCATGAAGTAGCATGAAGGGAAGTCACTCTAAGATTGGACTGGCTTCAACATT
TTATCGGTACCCATGTTCATGTTTACCAGGAGCTTTTCAGTATCTGGCATCATATTTTTTTTTTCCTGAGAAGTATT
GTGCTAATGCCAGTAGAGGAAACTTTATCATAAATGACAGGCTATTAAATGACATAGAATGATCAGGAGTTTGGCAT
TAGGGATTTACTTCTTTTTCGTTCACCATTCCTATAAAACAATTACATCCACTGTGATCTGAGATCGCAACACAGGT
CAAAGGCACTCTCATTTTGCCAGTAGAGATTTAGAAACACTGCACAGTTTGTCAGGTCGAGGACTGCCCAGCTCAGG
GGCAGTATCAAGATCTATTTCCTCACAGTGGAGGGAAGATGGCCTTTCTTGACCTTTCAATATAGAGGAGAGCACGT
GGAAGAACTAGGGGATGTTTTGAGCAACATTTAGGGTGTAAACTGGGAAGGGCTTGGAGACTCATTAGGTTTAGGGA
TGGAGAAGGAAAGATTGAAGATTAAGCCCTTGTTTCTAGCTTGGCTCACTGCTGGGGGTAGGGGAAAGGCATGGATG
TTGCCAATAATCAAGATGGAAAAGGAGAAAGAACAGTTGTAAGAGGATTTTGAACACGCTGAAAGTGAGATACCAAA
GGACTTAGACATCCAGGGAATGATATCTCTGGGGGGATTAGCTCTACATCTAAAGCTGGACAGTGTTGGAGAGAGGT
GGGCAAAGGCCGGGCAGGACCTATGGATTTTTGGAGTCTTTAGCAGAGAAGTGGTGCCAGCAGATGTGTTCACCCAG
CCACAGATTTAAGAAGAAGAGTGGGTTGAGCACGGAACCCCGGGAAAAGAAGAGATTTAGGTGGTGGCTGGAGAAAG
AGATATCTTGGAAGGATGCAGAGGAAGAAGAGTCAGGAAGTAAAGGAGATGAGGACTTGTCTCTGGGCTGAGAAAGG
ACTTCTAGTTCAAAATGATGGACCGCTCTCGTGCATAACCCATGCACATCTTCCAGACTCAACTGAAGTGTTGACAA
AACAACTGTACTGGGCTGAACTGCCTCAGAGAAGAAGAAATGAAGTGAGTCACTGACGGCAGTAGATTTGGACTAAC
TAATGTGAATCTGGAAAGCTGGCAGGTAAGAGGTGTCTGAGGAACAGGGCAGAGGCTGCAGAATCCCAGAGAGTCTG
TGGGGGGACATTCAGATGCAGGAGGAGGAGAGGTAGGTATCCTGGACGACAGCAGGGACACACAGCACAAAACGATG
CCATGAAACCGTGGACCCCTTCCCTATGCCTCAGCACGGCTCTGGGCCAAATGCATTCAGACAGTGCACTGAAGAAA
TGGGATCAATTTTGTAGGAAAAGTGTTTGAATGAGACCAGGGAGTGTACTTGTGATGCCCCAGAGCAAGGACCTCCC
CGTCTCAGTATTTAGGGGTCCCTCAGCCCAATAGCTGAACGCTCAACTACACAGCTTAAACTGATGACCCCTTGTCC
AAATACAACCTAGATCTTAGTTCATTGCCTATAGTCCCTTTAAAAAAAAATGAATTAGCTTTCCACATCTATAAATC
TGGGTATTACATATGAAAAATCCAGATTTCTGAGTTTTCTAGAAAATTCAGAAGTACAGCTGGAGCTCAGTAAGGGC
CACTCCCTTCCCATCTGGCATTTCCTGGCCACATGACACGGTCCCCACCCAGCTCCACCCAATTATGAGATCTTTCT
GTGGTCCGTTTATGAGCACTTGAGGATATGACCCCTGCCTTCAAGTAAAGCCTGCTGGATAACCACTCCAAACATAT
ACAGAAAGCCCTACCTCAGCTTGAAAAGGTCTTTGTTGTTGTTGTTGTAGATATAGATTAATCCCTTAATTCTTAAA
AGTCACCTACAGTGGAAGAAAGATCAGCCTGGGATAAGCAACACTGCATGCAACTAGAAGCCAAAGGAGCAACGCCT
TCGGGTGTCCATGGAAAGTAACAGCCACCCAGCATCATGGGCTCAGCCAAGCTATCGTGCAAGACCAGGCAGGAAAG
TACCTCCAGTTTAGCTCACGTGCAAATTTTCTTCCTCAGATTCTTAAGCAGAAGGTTCCACAAAGGAGGAAAGCGAA
GAAAGTGAAGCCATGGTGGGGTCTGGAAGTGGGTCAAGGATGTCTCTGGGTGGCAGATTGGCGGCAGACCCAGAGAG
GAGCCCACCCAAATTGGAGCAGGAGGATGGAGAACTCCAGGAGCCATGCGTCTAAGGAAGATGGAGACTTGTGTACT
AGAAAATATATTTATGAGTTTGAAAGGCAATTCACGTCCCTCCTCAAAAAGGGAATATGAGAAGGCTCCAGGTAGCA
AGAAAAGAGCTCTTCCAAGTACCGGCATAACCTCTTTAAACAAACCTCAACAACTAGAAATCTCACAAAATTCCTGG
GCAATAAAAGCACTGAGAGTCAAAGTAAGGACCACCATGTACGTGACAGGCATGATGCTTTGCCCCAGGGTGTATCA
AGTCTGCAAGAGAGCTGTGGCTTACTTTATCCTACAGATGTATTATCAAAAGCTATGGAAAAGTGACTTACTTTCAA
TGAAACATTTTATAGGAACTCGTGGTTTTAAAAATTCCAAAGATTATGGTTAACAGATAATTTAGAAGTTTTATAAA
TTTAAATTTGAAAGTAAAACAGTGGCTAAATACACAGACTCTGGAGATAGACTGCGTGTGGTCAAACCCCTGCACCA
TGATTTACTTGCTATAAGACCTCGGGAAAGTTATTTAATCTCTTGGTTAAATATGGCATTTTCCTTATCTGTAAATG
GGAAGTACAGTAATATCTGTTCATAAGGTGGCTGCTGTATTAAATGACTTAATATTTATGAAGCTGAGCTTGGCAAG
AGCAAGTTATCATGTATTTGGTGAACAAACCAAGACATTTATGATTCTTTTTTTTTTTTCTTTTTATTTTTAACAGC
CGAATCTGTGGCATATTCTGGGCTGTGGAAGTTTCTGGGCTAGGAGATGAATCGGAGCTGCAGTTTGTGGCAACACC
AGATCCTTAACCCATCGAGTGAGGCCAGGGATCAAACTCACATTCTCATAGAGACAATGTCAGGTCCTTAACCAGTT
GAGGCACAACAGGAACTCCTTATCAGATGCATTTTGCTCTAAATGAGTGTTTCACACAGGGTGTTCCTGTGTGTGAA
AACCCAGGGATTTTTTTTAACTCAGAAAGCTGGCAGTGGATTATTGGTTTCACTGAACTTTTGGCATAGGCTTTTCT
TCAACAGCAAGTGCTAACATACCAATGATTAAAATGTAGTTTAGGAACACATCTATTATAGGAAGCTACATTTACAC
CTCTACAATTAAGTCGCCACACATTCATGTGACACATGTAATATGCTTAAAGGTGGACTATATATCCTCCTAATTTA
TTTAGTGATTCATTTATATAGAATTAAAAATTACAATGTATGCTCACATATATCATGTCATTTGACTGTCATAAAAA
AAACTGATAAGGTGGCAAGAAGCTCAATAGAATGGAAAAAAACAACCTTTGGACAGGGATTCAAAGCCTCATTATTG
GTTATCTGAATCAGTCGGGGTGAGGCACCCTTCTTGGTCTTGACCTTGTGTCCAAAGCCCTAGTTCTTAACATCATG
CCTCTCTGCCGTAGGTGAGGGATTTGCTCAAAATTGGAGCTCAACAAAATATGTGTTGGTTTATGTTGACTTAACTC
CCTTTCCAGAGCCACACTGGGTTTGTTTGGGGAAGGAGACACCACTGGAGAGAAGGCAAGGAGGGCAGAGATCAGTG
CTTGCAGGTCTGAGAACAGCATAAGCAGGCCAGCTGTTTGGAAGGAAGCAGGTCAAGAAGCCAGTCTTTGCAAATGA
CTCAAAAAGAAGCAAGTACGGAGTTAATAGTAATGTTTCAGTATCAGAGTATTGGTTGTAACAAATGTACCCCAGTA
AAGTAAGATATTAACAATAATTTGGAGTTCCCATTGTGGCAAAGCGGAAACGAATCCAACTAGGAACCACGAGGATG
CAGGTTCAATCCCTGGCCTTGCTCAGTGGGTTAAGAATCCAGCTGTGAGCTTTGGTGTAGGTCACAGACGTGGCCCA
GATCCTGCATTGCTGTGGCTATGGCACAGACTGGCAGCTGTAGCTCCAGTTCAACCCCTAGACTGGGAACCTCCATA
TGCCACAGGTGTGGTCATAAAAAGCAAAAAAAAATTTATATATATATATAAACACTACTGTCTGTAATATCCTTGCA
ACTTTTCTGTAACTCTAAAGTTGTTCCAAAATAAAAAAGTTTATTTAGGAAGGAAGGAAGAAAGGGGCACTTCCACT
GGTATTCCTGCTTACTTCCTCATATGGATGTTCCCGGCTTGGTCTTTCTTTTGGAAAGGATAAATCCAGAAAGTCAA
CCAAATAGTCATATCCTCCAGGCAAAGGGCTGAAGTCCTCATCTGTCTCAATCATCTGTTCAAATGACAACATGGTA
AAGGGAAGAAGCATATCAATCTGGCGGTCAAGGTCCTTAGAAAATTCTAGAATGTGCAAGACCCAAGTGCCCTTAAA
TGATAGCAATGAAGCAGAATTAATACAAAAACTGTCTCTCCTCTTTGCTCTCTCCCACTGCCCCATCCCTCTACCCA
TCCCTCTCCCTCCCTCCCTCTCTTCTTTCTTGAACTGAATTCAAATCCTAGCCTTCTACACTAGCAAAACCACTTCA
TAACACTAACTTAAATAAAATTTATAGAGAAAATTATCATTATCTTAGTAATGAGATATCAAATTGGCTAAAAAATA
ATAAAATGTGGACTGTTTCTCATCATCACATAGTAGCTAAATATAAAAGAGTATCATTAGGAGTTCCCGTCGTGGCG
CAGTGGTTAACGAATCCGACTAGGAACCATGAGGTTGCGGGTTCGGTCCCTGCCCTTGCTCAGTGGGTTAACGATCC
GGCATTGCCGTGAGCTGTGGTGTAGGCTGCAGATGCGGCTTGGATCCCGTGTTGCTGTGGCTCTGGCGTGGGCCGGT
GGCTAAAGCTCCGATTCGACCCCTGGCCTGGGAACCTCCATATGCTGCAGAAGCGGCCCAAAGAAATAGCAAAAAGA
CCAAAAAACAAAAAAAATTCTTCCACCTACTATCCTTTTATTTTATGAAAGGAAAGATGTTTTCACACCTCAAAAAT
AGAAAGGACCTAATCTTGGAATAATGACAATTCGTCCAAAGGAAAGAGAGTTGACATCTTGGTGACCATACTCAGAT
GTGTGCTCATACTTATTTCGTTACTGACCAGCAAAAACTTTGTCACAGACTGTCACTGACCCCCAGGTTGAATTTTA
GGATTCATTGATTTTGAGGATGGCAAGTGTTGCCTGGTACCCAGTACTAATGTTCAGGGGTTGAAATTTAAACTTGG
AAATAGTCTTTACCCTGGAGGTAACTGATCTTTGTTCCTAAGGGTATGAATACTGTGCATTTCCCGATGCTTTCCCT
AAACTTTGCTCTCCAGGCACACATTCAGGCACTAAATATAAGTAGGATAAAATATAAGTATGGCAGGGATTCCCAGA
CCATTTTAGGCCTCCTCTTTCTCTTGCATCCCGCTGCCTGTTGCTACTTATTTTGCTTTTGTGGACATCCTCAGTTT
CAGTGACCAGCTTATAAGCTGAACCACTTAGCTGGTGAGCTCTGTGTGTCTATGTCAGGGCTAACTTAAGTTCTAGA
TCTAGGCTTACTTCCCAGTTGGTGCAATTCAGTCCTTACCCAGCTGCAGTCCTTACCTTACCTGCTTCCAGGCTGCT
ACAGGACACCAGCTCTGCAGTGGAGCCACCTGTCTGTCCCACAATTTATTTATTTTTTATTTTTTTATTTTTTTGCC
TCTTAAGGCCACACCTGCAGCATATGGATGTTCCCAGGCTAGGGGTTGAATCGGAGCTTCAGCTGCCAGCCTACGCC
ACAGCCACAGCAATGCAGGATCTGGGCTGCATCTGCGACCTACATCACAGCTGACAGCAACGCTGGATTCTTAACCC
ACTGAGCAAGGCCAGGGATCGAACCTACATCCTCATGGATCCTAGCTGGGTTTGTTAACTGCTGAGCCATGAAGGGA
ACTCCCCGTTTCACAGTTTATTTTACTTATTTATTTATTTATTTATTTATTTTGTCTTTTTGCTATTTCTTTGGGCC
GCTCCTGCGGCATATGGAGGTTCCCAGGCTAGGGGTCTAATCGGAGCTGTAGCCGCTGGCCTACGCTAGAGCCACAG
CAACGCGGGATCCGAGCCGCGTCTGCAACCTACACCACAGCTCACGGCAACGCCGGATCGTTAACCCACTGAGCAAG
GGCAGGGACCGAACCCGCAACCTCATGGTTCCTAGTCGGATTCGTTAACCACTGCGCCACGACGGGAACTCCCCCGT
TTCACAGTTTAAATAGCTGTCACTGCCATAACCAACACAACACAATACAACACCCACAAAAACCCAAAACAAACAAG
AACCAAGACACGGTGATGGAGGAAAAAGAATCCTCCAAAAGAAAAACAGAGCTGGATCTACATTTCATTCCCTACAT
TTTCAACATTCCCTACATTTTCAACAAAGGATTGTTTCAGCACATAGTCCAATACGCCCTCCGTCTGACAGTCAGTA
AGGCTCAATGAATGCTTATTGAGAAACCAACTGGAATACTAAGAGGTTTTCATATAGCTCTGTAATATAAGAAAACA
AAAACAAATAATAACTTCATAGCATACCCTGACCACCAGGTTATAATCCTTAAATCCAGCCCAAGTGAAGTATTCTT
TTATCCAGGATGAGTGACGAAATATTTCATCTCCTATAGCAGCATTCAAGATATTCAAATATGGGCCAAAATCCCAG
GAATCCTTGTAAATCTTAGTCCCTTCTGGAGGCTCTACGATGCCCTTGCTTAAAGACACAAAGGGGAGAGAACAATG
AAAAAAGAAAGCAACAAATAAGGAAGGCAGAAGTTTGCACTTCTACATCAACAGTCAACTGGATGAGCAGCTCTAAG
GCTGCTCAGATAGATGATGCCCAGGGGTCCCACAGATGTGCCTCAGGGAACATTGAGGAGTAGGGCCCCACCCCAGC
CTAAACCAGGTCAGCTCCTGTTAATTGCTTAGTGTGATAGCTCTCCAAGTCAGAATACATTTAAAGACGAAGTCTGG
AGTTCCCGTTGTGGCTCAGAGGGTGAAGAACATGACATAGTGTTCATAAGGAGACGGGTTCCATCCCTGGCCTCATT
CAGTGGGTTCAGAATCTGGTGTTACCTCAGCTGCGGTGTATGTCACAGATGCAGCTCAGATCCCACCTTGCTGTGGC
TGTGGTGTAGACCAGGCAGCTGCAACTCCCATTCAACCCCTGGCCTGGGAACTTCCATATGCCGCAGGTCTGGCCGC
AAAAAAGAAAAAAAAAAAAAAGATAAAGATCCATGTCCGGGGAAAAAAAAAGTTGGAATACCACGGATGTGGACCCT
TTGGGCTCAAATAACTAAATTATGAAAATGTTGAATATAAGTGGTCTTACTGATTTTGTGGACATCCGCTTATTCCT
GCCCTGCCCCCACCTCCATTAGACTACAAGTATGATGAAAGCAGCAACCATGACAGTACACAGAAGGGGTCCCATAA
ATATTTGTTGTACATAGGAATAACTCTAGCCTATCTTTGAGCTACACCTAGAATTTTGTGTCTCTCATATACAGCCC
TCTTATTATACTAATAATACCACAGCTGATAGACAGATGGGCTGACAGGAGACCCAGTCAGCAGTATGGACAAGAGT
GTGCTCTGACATCCCTAGAGCTGTCCATCCAGTGTGAAGATGGATCACTGCATGCAAGGTGGAATCTTGAGTCCTGG
CAATAGAATAGGACGTGATCTGGAGAAAGGAAATATGAGGAGGGAAATAGGCATCTGTGTAGTAAAGATTTGGCAGG
TAATGGTAGGTCCCTACATTCCACTTCTCCAAACACTGTTGGCCCAAAGCCGGAGATGCACTGGTTTTGGTGATAAA
TTATGTGTCAGATCCTAAAATGTCTAACTTCTAAATGAATCTCATATCTGCTTCTCTAAATCCTTGCTCCATCTCAG
CCAGCAGCCTCACTTATCTCCTCCTGGAAAAAAGCACAGTCTCCCAGCTGGCCCCCCTGACTCTAGGAGTTCTTCCC
CAGGACATGGTTTTTCTAAAACACAATGCAGTAATATTCCTTCTTTGCTTTATCGCTTTCTCAAGCTCTCCTTACTC
ACAGGCAAGTTCCTTGCCCTCCAGGCAAGGTCTTATAAGGACTTTCTGACCCTGGTCCAACACGGCATCCCTGTCTC
ATCCTTTTCCTTTACCTTCATTTACTGAAGGGGATGAATGACTTCATAAGGGAAGGACCTCTTCACAGCTGTTTCCC
CTGTACTTAGCATGATGCCCAAAGGAGCTCAATAAATCATTTCTGGAAGAATGGCATACATCTATGCACTTATTCAA
AGTAATTGTACTCACTAAGAGCATTGTAAATCAACTATATTTCAATAAAAATATTAAAAACTCAAAGTATCTGCACT
CACCAAACCTATGACATTATTTTCACCCCCTTTCTCCAGCATATCCCTCTGACTGGAACCTCAATCTCTTAATCACT
CTATTGGTAACCTTCTCCTGACCTCTAAGACATAGCTCAAATGCCTAAGATTGGAGGTTGAGCATTCCCTGTCCACA
TCTCCTGTTCTCTCTAGCCCTCTCCCTACCTCACAAGGCAGAGCTGAGCACTCAGTCTCCCGGAATCTCTTATACTT
TGTCTTACTACTGAGAACCTAACATCAACTCTCATTACCCAGAATGCTTTGGTGTGACACAATGATGCATATGCAGA
TTCCAGGGCTCTGCTTCAGATCTACTGAATCAGAATCTCAGGGGGTGGAGCCCAGGGAGCTGCATTTACCCAGTTTC
CTTGGGTTACTCTGACGCTCACTCTAGTTTGCGAATTTCTACCATAGGATGCGTCTGGGGAACTAGAGAGGGATAAT
GGAGAGAGTTCAGCAAATGCCAGGTGCCAGACTCTTGAATTCCCCACTAAAACGTGAAATAATTAAAATCTTCTCTC
ACCTTGAACTAGAGAATGAAAACTGCCTTTATCCTAGAGGCACTGGAGAGATCCTATGGAATTTTAAACAGGGAAGG
GAACGGGAAGAGTTTTGCACTTAAAAATCATTTCTTTGGCAGCAGTGCAGAGTTGGAGCTTTCAAACTTCTTGCCTA
AGATCCCAGGAAGAATATATTTTACATCAGGACTCTAGGGGTCCATATGCCAAGAGTATCTGTGAAACCAGAGTTTC
CTGAAATAATACTTACCCTTGTTATATGTGCTCAGGCAACATACTCAGGGTTGTTCTATACAATTTTGTTCTACTTC
TTTTTATTTTATTTTATTTTTGTCTTTTTTTTTTTTTTTTTTTTTTTTTTTTTTTTTTAGGGCTGCACTTGCAGCAT
ATGGAGGCTCCCAGGATAGGGGTCTAATTGGAGCTGATGCTGCAGGCCTACGCCAGAGCCACAGCAATGCCGGATCA
GAGCCACGTCTGTGACTTACAAAACAGCCCACAGCAATGCCGGATCCTTAACCCACTGAACAAGGCCAGGGATTGAA
CCCGCAACCTTATGGTTCCTAGTCGGATTTATTTCTGCTGTGCCACGACGGGAACGCCTATTTCCTTTTTCTAAATG
CTAGTTGTGATGCCATTGATTTCCTAACCCATCAATGAATCGTGACCAGCAGATTGAAAAAGGCTGGCATGGAGGAT
GGATCAGAGGACAGCGGGGCTGGGAGCACAGAGGCAAGTCAGGGGCCACTGCCAGAATTCTGGTTAAAAAAAAATTG
TGAGAGGCTGAATCAAGGCCACAGCAGAAGAGGCTGGAGGTGAGTGATGGATTTTTAAGAGATTTGTGAAGGAGAAT
TGACCAGATTTGAGCTGTGGGAAGTTAGTAAAAGGGTATAATCAGCTGACTGTGTCCCAGACCCCAGCTTTGCAAAG
GTAAGGCCAGGAGAAGGGTGTGCTTTTGGTAACCGTGTGCCCTGATCTCCAACAGAGTCACAGTCCACTTCTAAATA
ATGGTGAGGAATGATGGTTCCATCCGGCTCAAGACAAGTACTTATAAAAATACAGGTCTGGAACATCCACATTAATG
TTTCTGAACTGTACTCCCAGGGCACCGTTAATTGTTCAAATGGACTGTCTGGGGATTGGCGAGGAGGTAATATTTAC
ACTGATAGGAACACTAACTCTCAGGCTTATTGCTTTCTACTTGCTGAAGACAACTTATTTTTGAGCTGTAATAATGG
CCCTTCATAAAAAAAACTTTCTCACTCTTTATCCTGAAGTAAGGTTCTGAGACAAGGAAAACATTTGAGTAATTATC
TTATTTATTTATTTTTTTTTCAAGGCCACACCCACAGCATATGGAAGTTCCCAGGCTAAGGGTCTAATCAGAGCTGG
AGCTGCTGGCCTATGCCACAGCCACAGTAACGTGGGATCTGAGCCGTGTCTGCCACCTACACCACAGCTCACGGCAA
TGCCAGATCCTTAACCCACTGAGGGGGGCCAGGAATCGAACCCGCATCCTCATCGATACTAGTCGGGTTTGTTATTG
CTGAGCCACTACGGGAACTCCTAATTATTTTATAGGATAAGAAAATTATTATATAGGACTGTGAAAAAACTCAGTCT
CCCCCCCACCCCAGAGTTGAAAGATACTTATTTAATAGTTTATTTTATACAGTAAGACTCCCACTTTAAAGGGTGGT
GTGTAGATCTTAATGCATGACAAGCTCAGGATGCTAGTCAAGAAAAACTTAATATTCCTACAAACAGGGACCTGCCA
AGAGGCCATAGGTATGCCCTTTATTTTCTCATAAACATGAAAAAATTCAGAAATCATTTTTGTTCCCTGTAAATATT
CAAGTCAAACCTGTCTGTTGGGTCCTTTAGCATCCTACCCAGATCAAGAGTGGCTCCAGGTCTTGGGGTCCAGGTTA
CCACCTCAGAATTCTTCTTGATAAGATTGTTGAGTTCATTTGGGTCATTTTTGATGTTTGTTTCCTTAATATACCTG
ACAAATAAGAGCATTCCCATGTAAGGCAGTTTATTTTCAGATGACATTCTTATTTGAACAATGACAGAATTATTTTT
TATTTCTTTGCATTCCTACTTCCCAATCCTTCTTTTCTTACCCCAGGAAAAATAAAGACTATACTTGAGCTAATGTC
CCTGACTAGGGAAGAGCTGTTAGTCAAAGAAGGTTGACTCTATACTTCGTTTTTTAGTATAAGCATATAGTGTTTGG
AATTGAAGTTAGATGTACAAGACTATTATACATAATTGGTAATAGCACACTCTTGTATTTAATTTTTTTTATTCATA
CTCTCTGTTTTCAGGCTGCTTGTTAAAATAAGCTCCAGACCCCTACTAATCATTCTTTCTCATTTCATGTTGTTTCA
CAGCTAAATCACTCATTCAGCATATATTAACTTATGCGTAAACACGTTATATAAAATATCCAGCCATACTTGTCTGC
TGGGTGGGATTCCACGAAATACCCAGCAAAGGGGCAGTAAATTCTGGGTTGTAGGTCCTTCACCAGCCGAGCCTTGT
AGTTCAGGAGTTTCTTCCTTTCTGTTTTAATGAATTGGGCTTTCCATTCCTCTGGAATGACAGGGTTTGGATTAGTC
TTCTCTGTTCAGAAATCACAGAAAAACAAAAGTTCTAGTAGATTAGAAGTCTTGCAAGAGATAAAAATTGACAGTTG
AGTGATGCAGAAGTAGAACAAAGCTCCTTGTCATTAGTGGCTTTATTTTGCAAAGTTGGTTACTAGGAAAATATCCC
AAACTAGTCAAAGACATTGAATCCCCTCTTTGTTTACGGCAATTCATTTGGATCCAACTGAAAACACAGGGCAGCAT
GCATAGTTGTACCCTGGGTGCATGCATATTTTAAGGGCACTGTCGATTAACTCTCTACTAACATGGGCATGGCTTTG
TTATTTTGGTGGAATATAAAAGTAAAGTATGTTCATTACACTCTGGAGATGCACAGTGGTCAAGAGCATGGATGTTG
GAGTCAGTCAAGATCAAAATGCAGCTCCACCACTTCAATTCTTTAAGTCTGTTTTTCTCCTCTGTTGAATGGAATCA
TGATGCCTACCTCACGTGTTGTTCATTTGTTCGTTTGCTCATTCTTTCATTTGATCGATATTTATTGAGCACCTACT
ATGTGCCAGACGTAGTTCTAGGCACTGAGAATACAGTGGCGAGCAAGATAAAGCAGGTCCCTGCTCTCATGGAGCAT
TCATTCTAGTGAAAGAAGCAAATAATGAATAAGTAAATAAGTTCATTTCAAAGAGTGATGAGCTAGGAAGAAAATAA
AACAGAGCCACCAAATAGAGAGTGGCTGGGGTAAGGATGAGGACGGGTGGGATGGAAGGGCATATTAGAAGGGTAGT
TAGTGAAGATGACATCTGGAATCATAGACCATAGACACAGACACAGAAGAGAAGTTGCTGACCACGTGGTGGTCAGG
GGCAATAGCACTCTAAGCAGTAGAAATAGCACATACAAAGACCCAGGGCATGGAGCTACATGGTGTACTGAGTCTGA
GGAACGAAAAACAAGCCAGTATGGACTTATGCTTGTCAAGCAATGGGGGTATGGGCAATAAAGGAAATTGAGAAATT
AGGCAGGGCCCAGAGCATGTATGGTACCATGTCAGGTACTCCTTCTACCATTACTGTTATGAAAATTTGATAAACAC
AAACAAGGATACAGGGGAAAAAATGTTACCTATAAGCTAGGTGTAACCACTATGAACATGTTAGTATATTACAGACC
TTTTAAAATGTATGTGCATGTGCACATACTCACACACATACACATACTCACATAAGAACTGAATTATGCTACCACCC
TTTAGTAGGTATGTTTTGCCTCCCTAGTCACACTGTTAACCCCATAAGGACAGCACCTTCCCTCATCTCTCACATGG
TGATGCATTCTGGGAGGCAATGAAATCAGACTTACAGAAAAAAGGAAGGAACTGGACAGGTTTTCTTCTTATTGCAA
GTAGGGCATTTTTGACACATTACTAAACAGAGATTACTTACTAAAAACATTAATTTATTAAGCAGACATATATTGAA
CACTTACAATGATAGTACTGAGCAAAGGTATGAAAAAAATATACCACTTAACCATCCTCCCCATCCCAGCCCCAGAA
CCACCCTTAGACACAGAGCAGAAGAGCTTCTGCCTTGGTCCCCACATTTTTTCTAGCTTTGAGATATAACTGACATC
TAGTATTACATAACTTTAAGGTGTACAACATGGTGATTTCATGACATGCATGTATGGCTAAATGATGACCACAATAA
AGTTAGTTAACACCGCCATCACCTCACATAATTACCATTTCTGTTTGTGTGCACGTGTGTGTGTGTGGTGTGTGTGT
GTGGTGTGTGTGTGTGTGTGTGTGTGTGTGTGTGTGTGGTTAGAACATGTAAGATCTACTCTCAGCAACTTCCAAGT
ATATAGTACAATATGCTATCTATAGTTGCCATGCTGTTTATTATACCCCTAGAATTTATTCATCTTGTAACTGGAAG
TTTATACTCTTTGACCACTATTTTCCCTACCACCCCCCCAACCTCTCGTAATCCCACACTTTAGAGGGGCTTCCTTA
GCCTCATCCCTCCCCCGTATGAGCTTTCCACGAGGTCAAGGGTATGTATCCCCCTCAGGCTGCCCACACTCTGTTCT
GAACCACATACAAAGAGCACTTAAGCCTGGATTACCAATGTCAGACTCTTTCTGATCAGCTCTATGTTCTATGTCAG
GAATCCATTTGATCCAAATTATTCTTGATTTTTCCTGAGATTCTCCCTAGTCTCCTTAGTGTTTCATGCTCCATCAG
CATATTCTCAGCTGGAAACTTTAGTCTATATTTGTGACTTGCAAGTATGATTTCCCAATAAGATTGCACACCTCTTG
TGAGGAAGAACCATGTCCTAATTATCTTTGTATTGATTCACACAGCATTTAGCAAAGTGCCATGCCAACTCCTTGGC
ATCATTTTGATATAAAGAATTACCAGTAAATTTTCCACCACTGAAAGTCATTGGAAAGCCTGAAGCTCCTCCAGCAA
AATCACTCATCATTAATGCAACCTTCATAGGCAGCCTTCCTCCATTGGGTCTGGTGCAATCCACTGTATTGAGTATT
TTATGACCTGTGGGAAAACAAAATGGCATCGGACTCAAGGTGAAATCTTGAACACCATAGTTTGAATTCTCAGGCCA
ACAGTCTTCCATGTAAGTCTATATAATCTGCCTCATTCAATTATCGAAGAATTGCTCACATCCAAGGAAAAGAGAGA
GTAAGATTTGAAAATTTATACTCTTGAGTGACACATTTTGAACTTTCAAGGAAATAAATTCATTCTGTCTGATTCAG
TGGGTTCTGAATGAGGACACTTAGCCTGATTCCACTCCAGGATCATAAACAGACTACTTTCCTTAGCAAACTATATT
CAAAGGTTAAGCTCAAAGGATGCAGAGGAAAGTAATCAGATCAACACAACTCTCTCAACCTTTTGGAAATTCTTTTC
GATGATTATTGGGGTAAAGTGTATGATTCCATAACATAATAATATTCAAGATGAAAGTAAAACATTTATTCAATAAT
GTCAGTTTTAAGGAAATTACAATAGGTGAAATATAGGATATTTTTATCTGTTGCCTTCAAAAAAAACCTTTGCACCT
GTCACGGCATAGAGTACATTACTAATTGATTCTCTGTAAGATTATATGAATGACAGTCCATTTTCCTAAGACAGAGA
TAGAATATACTGTACTCTATGGAAAATGAAGAGGGAAGAAACAGATGAACATAGGATGATGTTTTGGATAACTATTA
TTATCCTTTCTACCAAGAGCAATTTTCATTGCTGATGAGGGTAAGAAAATACCTTTGTATTCCACAATAATGCAAGT
GTCCATCTCAGGATGAACGCCATCCATCAAGATCATGAATCGAAGATTTTTGTCTACCTGGAATTCAACAATAAAAC
CAACAACGGTTTACATCTATTTTGCTTTTAATTCAATATTTGAAGAAACTGTCCTCTCTTCTGGAAAGAAATCCCCT
TTTTTCAGAACTGGATTTGTTATCCATCAGAGTCATACCATGGATAATTGGAGAGGAAGACCATCTTATTTCAGCTC
AAATAGAGATTTACACAGGACCATGTACAGAAAAAGTAGGCCATTGTTTCTTTAGTCTTAAAATTTCTATCTCGCCT
CAAATTTATCCCAGAAAGGATAACCCAAACATGTGGAAAGAACACAGACCTGCTGCCATATTCCAAATGGCACTACA
TTGATATTAGTCAACTGGACGCCACTCTGATTCAGATTCCAAAATACAGGTCTTTCCGTGTTGCCAACATAAATGGG
AACATCTGGTCTTCTCTCAGCAAGCTTCTTCAGTGTTGGGTAACTAGGGTCAGAAAGATATACAGGTTGAAAGGTGA
AAAAATAGAATAATCTAGTATAAGAGAGAGTGTGATCCTTACACCAACACGTTGACCGAGAAGCAAGGAACTGAAAA
ACTAGACTCTCCCCAGAGTCCAAAAGAAGAGCTCTTTCCTCAAGGCTGACTATAACAGTGAGGAGGATTTCCTGGGA
GAGTCCTCTTTATTGTTAGAACATCCCATATACCACGGCATGTATATCAAACCAGGTGTGCAAATTCCGTCTTCCAC
ACTGATGCTGCTTTGTGCAAGGGTAGTTCTAACAGAAAGTACAGAGTGGAGAAGTTACGCCAAAGAGGTTTCTGGTT
TCATCTTGATTTTCCTTTTTTTTCTCATTCCTCAGTGCAGCTCCCTCCCAGTGAGAGAAAGGTCTCGGCCATATATC
TAAGAGAACGGATGGGTGCCCACCCTGGGGCAGTTTTTCAAACTTCGAAGGTTGATAGCCACACATGGTATACAGAA
TGAACTCCTTGTCCTTAAAGAGAGTTAGTCACTAACTAAGCAAGACAATAAAGTTTAGCACAGAGGAAAATGACATT
TACCTCTTGTAGCAATCCCAAGTCAGTACACAATGAACCATCCAAGCATTTTTGAGTACTTACATAAGTTGCCAACT
TTCATTTATTAGAATTTATTACATAAAAGGATTATATACTACTGTGTGGGTGGCAAAACATGAACAATAAACAAATA
AATGGCTCTGTAGGTATATTTCAATCATAGTGTTACACACTTTCACATGTTATTGTATTTGATTCTCAACAAAAGAC
CCTTTCATCTTTTAGTGTGCTTTTAATAAATGAGGAAACACACTCAGAAATATATGACTAACAAATAGTAAATTGGT
ATTCAAATTCAGGCTTTCTGATCCTAAACTTGGTGCTTCTTCTATTGAAAGGAAATTCTGGAGTTCCTGTTCTGGCT
CAGTGGGTTAAGGACCCGACGTTGTCTCTATAAGGATGCAAGTTCCATCCCTGGCTTCACTCAGTGGATCTGGCGTT
GCCCTGAGCTGCAGCATAGGTTGCAGATGCAGCTCGGATCTGCTGTTACTACGGCTGTAATGTAGGGTGGCAGCTGC
AGCTTAGATTCAACCCCTAGCCTGGGAACTTTCATATGTTGCAGGTGCAACTGTAAAAAAAAAAAAAAAAAAAAAAA
AAAAGGCAATTCCAACTCTAATGAATGTGCTATCAGGTTTAAGAATCATATTTGTACATAGACTATAATGTCTGGTG
ATATAGGATATTTACTCATAAGAAAAATATAAACAAAATCAGCATATCAGCACTTATTAACCATACTAATATTCAAG
TTCCAAAACTATATTTAATATGTAGAATCCAGAGGGGGAAAATCATTAGGTTTTCTTCTCTAAAAACAAGGGATTCA
AAAAAAAATCAAGGATTCTTTGAACATGTCTTTAATCTCTGGGTTAACATCTAAATCTTCCACTTTAAAGGGCTTTG
GGAGTTAGGATAAATGATTCTAACATGGATGTATTTTAATTTGTGATTTTTAAATTATTGACAATTCTTGCTGGTGT
CTATTAATAACACTATTATAATACTCATATATTTACATAATAAAATCACATTTCTTTGACTAAAGACAGTTTTCTAA
AGCATGCTGGCCCCCTCCCCCTTTGTTTTTGTGAACCAATAAGGCATTATTCAGTAAATAAAGGTCAGACAAGAGCA
ATGGAGATAAATGACTCTGGTGTTTATTAGTTGAGCAGGTAAGAGTCAAAAAACTCAGGGTCAATTCTGTCAAGGAA
ATAAACTCAAAGGAGTGAAAACTGCAAGGCTTGGTAACTTTTCAGCCATAAGCTATCTGCAATACACTACCCAACTA
AAGCATTGTGATACTACAGTTGAGAAGTGGCTTTTTAATGCCTGGCAACTTTGCCCACACAAGCCCCTGAAATCAAA
ATGAAATTGGTTTTCAGGACAGTGGTTGGGAAATGACCAGACTGAATGCCATAAAAAGTTCTTATCCTCACTAAAAT
GTAGTATACTCCCATAGAATATCTCTTGCTAGGACAATGGCAATAGCATCTTGTGACAGGCACTATAAAGCAATCGC
CTCCTTATCTTGACACTGTTCTCTCTAAGCAAGCTGTACAAATTGACTACCACACAACATAGTTATTACACAATGCA
TGAACTCAGGGCTCTCATAATCCTGAAATTACAAGTTTGGTTCCAGAACCTCCTGTGGGACAAAGATATCATGTAGT
AGACAAGTAGATTTTTAATCGTAGCACAATACTCCAGTGGGTGGTATTCGGTTTTTAAGTGTGTTACAGGTAATTTG
TTACTAAAGCTGTTAATTACTTAAGTTTTTAAACCCTTTCCTTAAAAAGCGAGAGAACACACCTGTGCCTTCGAGAT
CTCATGGACTTTCAATAGAAAAATCCAGGGGCCAGTCAACCAACAAACAATGTATTTTCCCTAACCATGGACATTAC
TATCAAAGTATATCCTTCATGTGAACTTGTCATGTAAAGTCACAGGAAAAAAAAATAAAGTTGAAATTGCTTCATTT
TAGAACACCATGGGCACTGCTGGGTATTGGCAACCTGGCAGTAGCAATACAAATTTCTCAATAAGGATGAACACATA
GGACCCTGTAATGAAGCCAGGGGGTTGGGAATAGGAGCATTCACAAATATTTGTAACAGTCCATTCACAAATATTTG
TGGTTTTTGTCAATGAAAGTTCCTCTTTCTCCCTCCTATTTGATCGCCTGGATTCAGGAAGTTTCCGTTTCTATCCT
TAGTATCATATGGCTCTGGTTTCACTGAAGGATGTGGTGGACTCAGGGTTCAAAAGTTGAGAGCTCAGTGTTGTCGA
AATGCTACAGATCAGGAGTTGGCAAAACACAGCGACCTGCTGCTGAATGCTAGGAAGGGCTTTTACCTTTTTTTAAA
GGGTTGAAAGGGAAATCAAAAGGCAATCATGTTTGGTGACACAGGAAACTGTTTGTGATATTCACACGTCATTGCCT
ATAAAGCTGAAGGCAATCAGGCTCCTTAGGACCGACTATGGCTGCTTTTGTGCTATAATAGTAGAGTTAAGTAGTTG
CAATGCCAACCATATGTCTTGTAAAACTCCAAACAGTTTACACTCTGGTCCTTTGTAGAAAATGTGTGCTGATTCCC
ACCATAAATGTTAAACTAAAAAAGGAAGTCAACTTTGATGATCCTTAAACTCAGAGTTTTACCAACTAGCCTGAGGG
TAGGACGTGAGAGGGTCCAGGGTTATTAACCCCATGCTCCTTTCCACAATAGCTCTTCTCACATCCCAATGGTATAA
AACAGGAAGGCACTTTAAAAAGGAGGCTATGCATGTTGCTATGGCAGTGGCGTAGGCCCGGGGCTACAGCTCTGATT
CGACCCCTAGCCTGGGAACCTCCATATGCCACAGGTTCAGCCCTGAAAAGACAAAAAAAAAAAAAAAAAAAAAGTTT
TTAAAAAAAGAGGCTATGCAAATGCAAGCATTTATCTGAATTAGTTCTCTTTTTATCAGCCCAAGCGAATCTACCTC
AGAATGAGCAGTGATTACAAAAAAAGCTGAAAACCAACAGTGCTTTTATTGCAGCATTTTCTTCGGAGTTGAGGGCT
CACCCTTCCTTACCTCAGGTGGTCTGAGTGCATGTGACTGATGTAAATTAAATCTGCGCGGCTCAGCCTCTCCAGCC
AATCAGATGGAGGCTCGTGTAGTAACCACCATCCTCGCGCAAAAGCAGGACCGATTAACCAAGGATCGAACACCATC
CTCTTGTCTCCCAGCTTGAGGTCCATGCAGGCGTGAGTAAGGTACGTGATCTGTTGGAAGACAGTGAGATTCAGATG
ATCGGATCATTACCAGCCAGAAAAAGGAACTGGGCTGGTTAGCAGACAAGCCACATGGGGGACCTTTGCTCCTAAGC
ATGTTCAATGACACAGGACTCAAGAAAGACACAGCAGGAGCATTTCCGTAGAACACAATTCCCAGCACAGGCATTAC
TTTATTAGAACAGAAATGCTCATGGTGGGTTTTAGGGGTCAAACCAGTTGATTTACCCAACTCAAATCACCTCCAAG
GTATTTAATTATGCTCTGTACCACAGAATATCTTTTGTTACCAGTCTTTTAGAACACAATTTACAAGGAAAGGGAGT
TACAGATGTTATGGCAGACCTCTGGGGATTTAAATGGTAGGGTGGCTGTGAATAGGTATAAGAATGACTGGTTCCAG
TGGGTGGACACAGTCATGCAGCCTGGCTGCACTGGCTTCTAAGGCTTTCTCACCTAAATTACTTGCGGACTCACTCA
GGATGTCAAGGTCCTTTGAGAAGGGTGAAAAACAATGACTTAGAGACAGGCAGAGACTACAGGATTCTAAATCAACG
CCTTACTCCCTTCCCATAGTCTGGCACGTCCACAGGAAAAATGAAAACACCAAGGAGCAGAGATAAGGTCACAGAAA
TCCAAATGTGAAAAGCCAGCAAAGAAGGTAGGGAGAGGTCAAGAAATCAAATGCAGGTGATTGTGCCTCTTCTGGGT
AGGTTCCCATTTGTCTCCTCAAAAAAGTAAGAGCCCATTTTTACAAGCTTCCCGAATACTCCAGAAAAATTAATTTT
TGGTTGTTTACCTCTCCCAAACTACCAAAGTGTTTTCTCTGGAGGAAATTCTCTCTCTCTCTCTTTTTTTTTTTTTT
TTTAGGGCCATACCTGCGGCATATGGAGGTTCCCAGGCTAGGGGTCCAATCTGAGCTGTAGCCGCCAGCCTACGCCA
CAGCCACAGCAATGCCAGATTCTTAACCCACTGAGTGAGGCCAGGGCTCGAACCCCTGTCCCCATGGATACTAGTTG
GGTTCGTTAACCACTGAGCAACAACAGGAACCCCGAAATTTTCTTTTAAAAGTGGAAAAATGCACAGAAAAGTTTGT
AAAGATCTTAGGGCAATGTGCAGAAACATGTAGCTGGCCATTTTATCTGACAGTGATCTGGTAGCAAGGGCAGTTTC
TGAACTTCCTCCCATAGCTGTGCATGACTCTCCTTTGGGACCTCTGCTAAAAGATTTTTTTTTTAATCTAGATATAT
TTCCTTGTAATCCTTGCCAAGTTCCTGAGGTTCCTAAATAATGTGCTCAAGAATTTAGAATAGGGAGTTCCCTGGTG
GTCTAGTGGCTAGGACTTGGTGCTTTCACCACTGCGGCTCAGGTTCAGTGCCTGGTCTGGGAGCTGAGATCCACATC
AAGCCACTGCTCACCATGGAAAAAGAAAAAAAAAAAGACTTCAGAATAACTTTATTATATGTCCTAACTAGCCACTT
CCAAGAATACTCAAGGTAATATAAGATGTAAAAAAAAAAAAAAAAATATATATATATATATATATAAATTGATATGT
TAGCTTTATTTGTGTTTTTAAGAATATTATAATTTAACATTTCCTTACCTGCACTTCCCCAAAAGCCAAATCTTCAG
GAGATCTGGGTTCTGAATCCCACGGGTTAGGAGGATTTAGTTCTAGAAGCAAAACTCCATTTTCTTCATCCTTTTCT
ACAACTAGAAGCAAAGGTGGACAAATCTGGATAATCAACCAAAAAAATGACTTTTAAAAAGCATCGCTAAGACAGAA
ATGCATGGCTCAAGTACATGGAGTAGACAAATCAAAGCAAAATCAAAATAAAAGGCAACGCTCATTTGGGTCAAGCA
ACATCTGCAGAGATGAGGGCTGAAGACCAATACTGTTCATCTCGCTATTCACATTCCACGTAAGGAACTCATGAGAT
CGCAGATGTGTCAGAGACACAGGCACACCACCACCAACTTCATTACAATCAAATGAATGATTGATAGAGATGAGTTC
AAGGTGCTGTGGAAGTGTCTCGGAAGGAAAACCTTGTTTGGTTGTAAGAGTCAAAGCTGATTTCAAATAGGAGGTAA
TCCTCCAGCTGAACTTGAAAGACAAAGTATTTGGGGGCTGACAAAAGAGATGTGATGATGGGATATCTCTTTTGGAT
AAAAGATAAAAGGACAACATAAAAGATAAAAGAACAGCATGTGCAAAGGCATGGAGGCATGGGAGAGCTGGATGTTC
ACAAATGACTGGAATTTTATGACCAAGGAGAATGGTGTCTGAACCAGGTGGGAGAGACAGGTAGGTCAGAGTGGGTC
ATGAAGGACCCTAGATTCCCAACTAAGGAGGCGTCTGGATTTCATCCTGTGGCAATGAGGGGTCAATGAAGAATTTT
AAGCAATTGTGGCAGGCATGCTGGTGGCTTGCGCAAAACCTATTCTCTCCTTCTCCCTTACTATTAGCATCCTAATT
GTGTGATGGTACACCTATTTAAAGATTTCCCAGCCCCCTGGCAGTTATGAGTGGCTATGTAGACCTAGCACTATGTG
CAGTTTACATAGTTCTGGCGGGTGAGACGTAAGCAGACGTCTACTTCAGAAGTCTCACGGGACTTGCAGGAACACAT
TTATTTCCCCGACAAAGAGGGACAACTCAAGAGACCAGCACTGTCTCCCCTTCATCCCTTCATATTTCCCCCTCTTG
TGTGGAATTTGACTGCCATGCTTGGAGGAGCACAAGCCATCTTGAGATGCTGAAGAATAGAGCCAGACACTGAGGAT
AGAACAGGAGGTGATAGGGAATTTGGCTCCTTGATAAACACAGAACAACCATAATGCCCAGGATTACCTGCTTGGGA
TCTAAGAAAAACAACCTCCTATATGATTGAGCAACTTTTGCCTGGTTTTTCTATTGCACTGGCTGAAAGCAATACCT
AAGTGCTATAGCAAGGGAGAATTAAAATCAGAACTTAATTTTAGAAAGACCCGCTGTGAGGCACATGGAGAGGATCA
ATTGGAGGGAGGCAAGACCATGTTTGAGAGTCCTCTCTGTTGTTCTGGAAGGCTATCAGCAAACCACTAATGGACAT
GTGCTTGGGAGACAGATGGCCTGTTTCTAGCCCTCACTCTCCCACTTAATAGCTTATTAGCTAGAGGACCTTGAGCA
ACTTATTTGACTTCTCCAGTGTTTTTATCTCTAACCCTGGCTATCTCCACACACAGTTAATCCTATTACTGCCAGCA
ATTTTATTCATTACTAAATGAAAGCAGATGAGGTCCCAAGCCAAAGCAAACCTTGTGGAAATGGCATTGCCGCCCTG
CCCTCAAAGACGAGCACTTTCCTACTTTATTCAAAGGACATTAAAAAATGTTTTGTGGGAGTTCCCACTGTAGTGCA
GTGGGTTAAGAATCCAACTGCAATGGCTCGGGTAGCTGTGGAAATGCAGGTTTGATCCCTAGCCGGGCACAGTGGGT
TAAAGGATCCAGCATTGCCACAGCTGCAGTGTAGGGCACAGCTGCAACTTGGAGCCTGGATTCAACCCCTGGCCCAG
AAACTTTCATATGCTGTGGGCATGGCCCTTTAAAAAATGTTTTGCTTACATTTTCCAAATGAATATTAATTATACTC
ACTTTAAGACAACTGCTAGTGGAAGAAACTGAAGTAAAAATTACCCGTAAAATGAAAAATGGCACAAATGAAACTTT
CCCCAGAAAAGAAAATCATGGACATGGAGAACAGACTTGTGGTTGCCAAGAGGGAGGAGGAGGGAGTGGGATGGACT
GGGAGTTTGGGGTTAATAGAGCAAACTATTGCATTTAGGGAGTTCCCATCGTGGCTCAGTGGTTAATGAATCCGACT
AGGAACCATGAGGTTGCCGGTTTGATCTCTGGCCTCACTCAGTGGGTTAAGGATCCGGTGTTGCCGTGAGCTTTGGT
GTAGGTTGCAGATGAGGCTTGGATCCCGAGTTGCTGTGGCTGTGGTGTAGGCTGGCAGCTGCAGCTTCAATTTGACC
CCTAGCCTGGGAACCTACCTATGCCAAGGGTGAGGCCCCAGAAAAGACAAAAAAAAAAAAAAAAAAGACAAAAAAAC
CCCAAAACACATATACAATAGATGCAAACTATTGCATTTGGAATGGAAAAGCAATGAGACCCTGCTGAATAGCAGAG
GGACTATATCTAGTCACTTGTGATGGATGCATATTATCTGCATCCTGGGCTGCAATTTCCTGATCTGTCAAATAGGA
TTATGATACATACTTTGCAGAGTTGTTGTAGGGATTAAGTGATATAATAAATCCTAAAGTGTCACTATGCCTAGCAC
AGAGAAGGCACGTAATAAATGATAGTATTATTATGGCAATTATTTCACCCTCAAGGAATAAAGAATTAAAAAGGAGG
TTCAAGACTGAACAAACAGGAGTTACTATCATGGCTCAGTGGTTAACGAAACTGACTGGAAACTCAGGTTCGATCCC
TGGCCCCGCTCAGTGGGTTAAGGATCCGGCATTGCCACGAACTGTCATATAAGTTGGACCCCGCTTTGCTGCAGTTG
TTGTGTAGGCTGGCAGCTGTAGCTCCAATTTGACCTCTAGCCTGGGAACCTCCATATGCTGTGGGTGCAGCCTTAAA
AAGACAAGAGACAAAAAAAAAAAAAAAAAAAAAAAAACCCACAAAGATTCAAGAAACAAAATTATATGCTAGCACAT
AACCAGTTCAAAAATACAAGGAATTGGGAATTCCCATTGTGGCTCAGCAGAAACGAATCTGACTAGTGTCCATGAGG
TCCATGAGGAGACAGATTCGATCTCTGGCATTGCTCAGTGGGTTAACAATCTGGCATTACCAAGAGCTGTGGTTAAG
TCACAGATGCAGCTTGGATCCCATGTTGCTGTGGCTGTGGAGTAGGCTGGCAGCTGTAGCTCCAGTTGGACCCCTAG
CCTGGAACTTCCATATGCCACAGGTGCAGCCCTAAAAGCAAAACAAAACAAAACAAACAAACAAAAACCCAAAAAAA
CCGACCAACAAACAACAACAAAAATCCCAAGGAATTACAGGAGACTTTCAGAAAACTACATCGATATCCATGCTTAA
GGATTTTCCTTCTTTAGAAGTGTTCTTTTTCAAGAAAAGCAGGAAAAACTGAGTCTGCAGTTCTTAACTATTATTTC
AAAGCCAATACCATAAAAGTTTTTATGCCCCTTGCTCAAAGATAAATTGCATTTATGCACTGAAGAAAATCATGACA
TCTGCCAACTGCCTGCATCTTTATAGAATGTGGTATCCTTACTTTGACCACATAAACTAATGACATCTAAGTTATTT
GGATTATGACTTAATATTTAACCAGAAGAACAAACAAATGGAATTCATTAAAATTTTTAATAGGGAGGAATAATGAA
GAGGAATTATAATAAAAACATATTAGAAAACTATAATAATTAAATCATAGATAATTGGCATAAGGACGAAGAGAGGA
TCCTAATTAAATAACAGTTTAATATAGTCTAAGAGAAGGACCATAAATTAGTGGAAGAGGAAGGGCTGCCTGATACA
CAGTGCTGTGCAATGGTTAGTTAAGTATTCCAGGTGCTTAAAGACACAAAGAAAAGCAACCAAGTGCTTAAAAAGTA
TGAAAAAAATGGCATATCACAGGGGGGACTTCTAAGTTTAATAGCAATGGAAATAATTCCAATGGAAAATCTTAGTA
GATAGAAAAGTAAAATGAAAAATTTCTACCACCTAAGAAAATGGGCAAACACACTTGGAATATATAAGCATCGTATT
TGAAAAACAAGTATAATTTAAAACAATGATGCTATTTTTGGTTCAAATGAGGAACGTTTGAAAAACTAGAATGCCCT
GAGCTGATAAGGAATGAGGAGAAAAGGCAGGCTGATTAAGTAGTTAATGGGAACAAAAATTGGTTGGGTTCTAAAAA
AATGGATTATAATGCAATACACATTAAAGAATGGGTAAATGAATAGTGGACTCATTCATTCATTTAGGACCTCAAGT
TAAGAGGATTATGTTAACCATATTTCTCAGTTCATGACACATTATATTCAGTCCAGGCAGAGCTACTTACTTACTCC
CTTTATCTTTGTTTTCTACTCTTCTTTACTCTCCTCCCCTGTAGGCAACCATTTGAAAGTTCATGCAAAATATTTAC
TACATTGTATGTGTGCATCTTTAATTTTTATAAATGGTATTGGGTTTCCAGGCTGTTTCTTACTCTTTTTCATTCAA
ATCTATGTTTCTAAGATACATTCATGTTGCCATGTGGACATCTCATCTCTAACTGGAGTTTCACATACCCTGGTGCC
ACATTTTATTGATTCATGCTCCCAGGGGTGGACCCATAGATTCTGCCACAACAGGATTTCTTTGGTACATAAACAGG
CGTGGGATTGATGGGCCACAGTGTATTCATAAACCTGCTCTGCCTAACCACTGTCAGATTACTTTCCCACATGACTG
CACCGGCCATACTCCCACCACAGGCATGACGATTTTTATATCCTTTATCCCTGACATTTGATATCACCTTTGTTTCT
AACTTTTTATCAGTCAAAAAGATGTAAAGTAAAGCACCTCATTGCTTCAGTCTGTAGTTTTCTAATAATTAATAGGT
TTGAGCATATTTTCATGTGCTTATTGACTTTTGGAGATTTTTCTTTTGTAAAATGCTAGTTCATATCCTTTCTTAAT
TTTTGTATTTTCTTAATTTTTATATTGGGTTTCCTATCTTTTTCTTGTCGATTTGCATTACTTCCTCCTATAAGCTG
GATAATATTCCCTCATTGGTTGTAAATATTGCAAAATAATCACTCAAACTATCATATGTTCTTTAACTTTGTCCATG
GGGTCTTCCAGTTCATAGAAATCTGTAGTGTATCGATGATATCTTATTCACTAGGTTTGTGTATATGTGTGTTTCTT
TTTTCTTTCTTTTTTCCCTTTGGGCTGTACTTTTGAAGTATTGTTTGAAAAGTCAAGAAGTATCAGTAATCTCTAGG
TCACAAAAATAGTCTACATTTCTTCCATTACTTTCATAGTCTTACCTTCCTCATTTGAGCTATCAGTCCATGTGAAG
CCCATCTTTATGTTAAAGTATGAGGTGTTAAAAAAAATGGGCGGGAGTTCCCGTCGTGGCACAGTGGTTAACAAATC
CGACTAGGAACCATGAGGTTGCGGGTTCGATCCCTGGCCTTGCTCAGTGGGTTAACGATCCGGCGTTGCCCTGAGCT
GTGGTGTAGGTTGCAGACACGGCTCGGATCCAGCGTTGCTGTGGCTCTGGCGTAGGCCGGTGGCTACAGCTCCAATT
CGACCCCTAGCCTGGGAACCTCCATATGCTGTGAGAGCGGCCCAAGAAAATGGCAAAAAGCCAAAAAAAAAAAAAAA
AAAAAAAATGGGCGAAAGCATGAGTTAGTCATATCCTTTTGCCAGTAATTCATTTGTCTCACAGAAACAACTCCAAA
CACAAAGCAGCTCTTACGCACAATGATCACAGTTTCGTTTTGATGGAAAAAAAAAATTATGAACAGTCTAAATTTCA
ACAACAGAAAAATGGCTAAATAAATCATGTAAGTTAATATTTAATGTAAACATACTTTATAATTGTGTATATATGGA
ATCTGACCTAACATGACTACTATAATAATTTTAACAAGACAAAAAACAGGATAAAAAAAGTAATATATAAAATAATT
ACAATTGACTGGAACAACTAGATAGAAGATGAACAAGGAAATAGAAGACTCGAACAGCACTATAAACTAACTAGACC
TAACAGACAAAAAAAGCACATTCCACCAGCAGCAGAATACACATTCTTCTCAAGTACATTTGGAATATTCTCCAGCA
TAAACTATGTTATATAAACGTTTCAATAAATTTTAAAAGATCAGTCATACAAAGTATGTTCTCTGACCACAATGAAA
TGAAATTAGATACTAATAAGAGAAGAAAGTTGGAAAATTCACAAATATGTGGAAATTAAACAACATACTTCTAAATA
CAAACAGTTTAGGAAAGAAATCACAACAGAAATTACAAAATGCTTTGATACAAATACAAATAAAAACATAACATGCT
GAAACATAGAATGCAGCTAAAACAATGCAGTGCATAGAAGGAAATTTATATCTGTACACACCTATAATAAAAAGAAA
GATCTCAAATAAAAAAACTAAACTTCCACCTTAAGAAATTAGAAAAAGAAGATCAAACTAAACACAAAGCAAACAGA
AGGAAGGAAATAAGAAAAAAAATTAGAGCTAAATGGAATTTAGACCGGGAAAACAAGAGAAAATCAATGAAGATAAA
TGTTTGTTTTTTGAGGGAGTTCTCGTCATGGTGCTTCAGAAATGAATCCGACTAGGAACCTGAGGTTGCAGGTGTGA
TCCCTGGCCGAGCTGTGGTGTAGGTCACAGATGCAGCTTGGATCTGGCATTGCTATGGTTGTGGTATAGGCCAGCAG
CTGTAGCTCCGATTAGACCTCTAGCCTGAGAACTTCCATATGCCTCAGGTGCAGCCTTAAAAAGCAAAAAAAAAAAC
CAAAAAACAAACAAAACAAAAAAGTTAGTTATTTGAAAAGATTAATACAATTACAAACCTTTAGCTAAACTGACCAA
GAAAAAAGAGAAAAGACCCAAATTACTACAGCCAGGAATTAAAAGGGGGATATTACTATCAATCTAAATAATCCAAA
TGAAATGGAGAAAGTCCTAGGAAGAAACAAATGAACAAAACTGACTCAAGAAGAACTAGAACGTCTGAGGAGCAGAC
CCATAACAAATTAAAGAGATTTAATTAGTAATCAAAAAACTTTTCACAAAGATTAGCCATGGCCCAGATGGCTTCAC
TGGTGAATCTGACCAAATGTTTAAAGAAGAATCAATACCAATATACTTCACAAACTCTTCCAATAAATAGAAAAGGA
GGGAACACTTCTCAATTCATTCTATGAGAGCAGTAATTATTACTCTGATCCCCAAACCAGACAAAGATATCACACAA
AGAGAAAACTACAGACCAATATTCCTTATGAATATGGACATAGAAATCCTTAATTGAATATTAGCAAATATAATTTA
GCACTATAAAAAAGAATTATGACCATGAGCAAGTGGGGTTTATGCTAGCTTGATTCAATATAGGAACATCCATGGAG
ACAGTAAGTAGATTAGTGGTTGCCAGGGGCTGAGGGAAGAAGGGAATGGACTGCTAATAGTTAGAAGGTTTCTTTGG
GGGATGATGTGAATGACCTGGAATTATATAGTGATAGTAATAGCACAACATGTGAAAATACTAAAAACCATTGAGTC
AAACACTCTAAAAGGGTAAATTTTATGGTACCTGAATTGTATTCCAATAAAAGGAGAAGGAGGAAGAAGAGGAGGCA
GGGGAGAGGCGGGGAAGGGGACCAAGGTGACAACTGGCAGATACCAAAACACTGATGGAAATGTAGGTGAGAGTCTT
CTTCCTTCTACTTTCCTAACATCTACCTTTTTTAATGATGACCATACAATGTTATTTATTTAACAATAAAACCAAAT
AATCTCAGCTCACATGGGATTGAGCCATCCTTTTCTTTCTTGGGATGTGGTATGAAATCACTACAGTATTGGTAGCA
CTGTACTGAAAAGTGGGTTCTGTTAACAAAATTTTCTACTCTCACAACATTACCTTACTGGAGCAGAGGCTGAAAAC
TGCAGTGGGTCTTGTTATTTCCAGTCCTCCACTGACCCTACTGACAACTCTGGCCCTGCCCTTCACCTGCCGTGGCA
GTGAACATCAACGCTTTGCATCATTTCCTGGCCTCAGTCTATTTTCCAGTTTACCCAACTTTCTGCTGGGTGGGAAA
TCCCTCCTTCCTGCTCCACAGGACCCAGTCACAAGGCATATGGCAGACTATTTGAGTCATACATATACAAGCAAATC
ATTACTCTGTACTCTGTCGTAACACGTTCTGAACATTTAACAGATGTTCTTTCAACAACCCAGTAAAATCACTACTA
CCAATATTATCTCCCATTGAGGAAACTAAAGAACAGAGACTAACCCACCTAAAGTCATTTAATTGCATGTTTGAGCA
TCAGGATATGAACCCACGCTAGTGAGCCCCATTCACTCTTAACCATTTTGCTAAAAGGTCTCACTATAGGTCTTATC
CAAAAGACTTAGCTCCCTTAAGGAGCTATAAGTTTCTGGGTTACATACTCATAAAGTAGATGGTCAATTGTCCTCTC
ACCTACACAAACAGTTTAAGACAGTCAAACTTTTGCTTCTTATCTCTTTTTTTTTTTAATCAGATGAATTAAATAGT
ATTTGTACAGCACATGTAACCAGTTCCTGCTAACAATGTGATCTGAAGATTTCCTAGGCTAGGTCAACAGACAAAGG
GTGGGGGCTTTCTGGCAAAAGAAGGAAATGGTTCAGGCATCCCTTTGAGGGGCAAGGTGAGAATTAGTCAATATTTC
CAAAAGTCATTTAATTGTGTTAGATCAAATCTACTTTTTTATTTATATAACAGTCATTCTAAAACAGTGTGTAAAAG
CAGTTTTAAGAATCTTCCCAAGTAACTTTTTATACTGATAAAGACATTTTTAATCACTTAGAACAGAGACAAATTTA
TTCCTATGATTAAGCCCTTCTTACTCATATTTCTATAGGCTTTCTTGAGTAGGAAGAAGGAAAAAGTAGAAGTGGAG
CCAGCATGAGAATCACACAGAAGCTGTAGCCTCTAACGTGTGCCAGAAAGAGTCATGGAATTTGAAGGACTTTATTT
CCCAACTGGAATTGTGAGTTTCATTATAACGTCTCATTATATCATCTCATTTACGCCGACTCTATCTTATCCATCTT
TGTATTTCTTAATACCTAGTGCAATGTTTACACATGGTAAGGTCTCATCAAATACTTACTGAACAAATGAATGAATG
AAGGGATTTTTTAGAGAAAACTTGCCTAGAATTTTCAGTGATGGTTACTTTTAAAATACCTCAGTTTAAAATCAGAA
TGCATCCAAGGCTTCTAATGAGATTGGAAACAAGTTGACAAGAGGGACCCCAATGACAGTAACAGCAGAAAACATTG
ATCAGTATTGATGGTATTTACCCAGTTCGTCTTGACAGAAGCTTCCAGGAGGATTGATATACTTCATGCTGCTTACA
TCTAACTTCCAGTTGTGTTTTGTGCATTTAACAGACCTGGATGGAAAATTGTACTTAGGTTTATGAAATGGTGAAAA
TAAATATTAATCTATTTAAGGCTTAAATGCATTATTCTGTGATCAAAGTAAACGACTGTAGTTGGTTGAACACAAAA
CTCATGAAAGGAAAAAAATAGCTAATATTCAAATATCCAAGGAAATATAAACTCATCATCAGTAGGTGATTTTGAAA
GTGAAGATATTTTTTCCTTGTATTTGATTTTTGTCAGTTTGATTTGTATGTGACTTTGCACATTTCTCCTTGGGTTT
ATCCTGTATGAGACTCTTCGTGTTTCCCTGACTTGAGTAAAGTGAAGATAAACACCATGGCACAAAATAACGTGTTA
GAGATCAGCAGAGCCATCAGAATAAAGTCTGCTTTGGAGTTCCAACTGTGGCTCAGCAGGTTAGGAACCTGAGCAGT
ATCCATGAGGATGTGTGTTCAATCCCTGGCATTGTTCAATGGGTTAAGGATCCAGCATTGCTGCAAGCTGCAGTGTA
GGTCACAGATGCAGCTCAGATCTGGCATTGCTGTGGCTGTGGCATAGGCTGGCAGCTGCAGCTCTAATTTGACCGCT
AGCCTAGGAACTTCTATATGCTATGGGTGCAGCCCTTAAAATTTGTTTTTTTTTTTAAAGAATAAAGTCATCTTTAA
GGATGACTCTCATACAAAAGCTAAGCTGAGTAAGATCCAAGTGGGGCCAGTATAAGGAAATAATGTAGTAATAAAGA
TTATCTGTGATTTAATAGTCACACTATAACCCTTGGCCCCTAGTATAGTGTACTAAACCTAAGATCAACTCAAATTT
TCATTTGTCTAAGAAAAAAGACTTCCTGATTGTTTAAAGATTTCTGATCATGGTTGCCAGATAAAATACAGGAAAAA
TATAAATTTCAGATAAATAAAAAATAATTTTAAAATGTCTTACACAATATTGAACATATATTGGAAATTTGTTTATC
TGTAATTCAAATTTAACTACTCAGCTTTGCATTTTTATTTGTTAACTCTGGCAACACTGCTTCAGAATGAGAATCAG
ATTAATTGTAGCAACAAAGGAGGCTTAGTAATATTTTTTCCATTTCTTACCAGACGGTGATAGGGATGTGATAGTTG
GAGATAGGGCCTAAAAGTTCCATTTCCTCTCCATATTTGGTAGTCTGTCTGGCTGTCTTTCTTTCTTTCTTTTTGCT
TTTTAGGGCTGCACCTTTCTTTTTGCTTTTTAGGGTGGCATATGGGGGTTCCCAGGAGAGGGGTTGAATCGGAGCTG
CAGCAACACCATATCCTTAACCCACTTAGCGAGGCCAGGCATCAAACCTGTGTCCTCATGGATACTAGTTAGATTCA
TTTCTGCTGTGTCCCAGTAGGAACTCCCATATTTTGGTAGTGTTTCCAGTCAAGTTTTTTTTTAAACAGTTCAAGAT
TTTTTTTTTTTTTAACAGACAAATATGTCTTCAACCAGAAATATCAGATTGTTTAAGCTAACAATGTCTATTTTCAC
TTATATATCAGTAAACTATGCTGATTTTTTCCAAGCTTCATTACAATCAAGAATTTTTAATGCTCTTTTCTAGTAAC
AAGGCAGAAAACATATTCAAACTTCGACTTATGGAGGATATTTTGTGACACTTCCTTTCTCATCAATGAGTAACTAA
CAACTATCATGGCTCAGAGGTTAACGAATCTGACTCGTATCTATGAGGACGAGAGTTTGATCCCTGGCCTCGATCAG
TGGGTTAAGAATCCAGTGTTGCCGTGAGCTCTGGTGTAGGTCAAAGATTGGCTCGAATTGTGCATTGCTGTGGCTGT
GGTGTAGGCCAGCAGCTACAGCTCACATTGGATCCCTAGCCTGGGAACCTCCATATGCCATGGGTGCGGCCCTAAAA
AAAAAAAAAAAAAAAAAAAAAAAAAAAAAAAAAAAGATGAAATAAATAAATTAACAAAAATTGAAAACATTCCAAAT
GCAGCTATTCAGCAGGCTGGGTCGTTAAAGGAGAAATGTGGCAGTGTCACAACTGCTCATGGGCAGTAGGCAGAAAG
GAGAGAGAGGACAGCTTCATGTGCCAAGAGGCTGTGAAATTAGATTGACAAAATGAGGACCACAGCTTATGAGAGTT
CCTGATCTTGATTATGTACAAAGAAGAAAAATGGCTGAGGAAGGGAAGGTGGAACAGGTAGGTCACTGCCCTTGACT
GTATCGTGGAAGAGATATTTCAGGTGAATTGCTGCACAGAGAGCCTAAGTAGAAGCAGCCAAATTTGGAGAGATGGA
TGGGGGAGTGTACCATGTAAACTGCTCTTGGGATGGAGTTTCAGCATATAAATGCTTGGGGAGCTGTATCTGGGAGC
AAAGCTGGGTGAATCTGGCTCCCCACCTGCAGCAGAGCTAAGATGGTGCCATCTCCATGTTAGCCTGCCAACAGAAT
AGGTTGAAACTGGGATCGTTCACCCCCTAAGGCTTTGGGTGAAGGAGAGGAAGACCAGTCTGTGGCAAAGCAATTAC
CATATTAAGCTGAGCAAGCCAGATTCAAGAACAGCCTGAATTCCTGTAAAGAACCTCTGTTCCTAAGCTACGCAAGA
TCATGGCAGAGTAATAATAATAGCAAATGTAAGTGACATTTATTGAGCATGTATCATATGCCAGACATTATTTTAAG
TGCTTTAGTGTATGAAATCACTCCATCCTCTCAGTAGCCAGACAGAGAAGGCTTTGTCTACTTTCATTTTCACTTTA
TGAGGAAGAAGAGCGAGGCCCAGAAAGGCTAAGAAATGTGTCCGAGGTCACAGAGCTGCTAAGTGGTGGAGCCAGGC
TTCTAAACCAAGCAGTTTGCAAGGAAAGACCATGCTCTTAATCATAAAGCTGCAACACTCCCTTAAACAACTGGCTA
AGACAACACCACAGGACATGGCCCACTAAGGAGAAAAAAGGACAGAGAAAAAGCAGAGTCCCCGGGCCACAAGTCGG
AAGACCTCAAGGCCTGCACGTGCCTGCAGAAGCTTCTTGGTGACAGAACAACCTATGGCTGAGGTCTCCCTAACTTG
AAACCACCCAGAAGATGCAAGGGACTCAAAAGCAGTCTGTCAGCAAACAACCAAGAGGTTCTTCCAGAGTAGGCTGC
CTACCAAAAGTATGTCCCATGCAGTGCCTGAAACATATCTAACTAAAAATATATTCGTTGTTTATCTGAAATGCAAA
TTTGACTGGGCACCCTCTATTTGCCTAATCTAGCAACCCTATCTGCAGAGCCAAGCAAGCTACAGGTATGACAGCAC
TTAACCTGGGAGCTGGGCCCTGAAGCTAAGTATGCAGTGATGCAAGTCTGTGGGCCAGTGTAAGAAGATTCCAGACT
TGGGTGGTGATCTTCTATACAGTTAGAGCAGGGAGTTCTTGGACAGCTACCAGTTACCTCTGAGTCCATTCGCACTA
AACTGCCCACAGATGACCTGAGAAATAAGATTGACGACACGACACGGTGGAAGACAAGCCTAATATGGAAACGGCTG
AAACACTACGAGAGTCAAGTTAGGCTGAAGCAAAGCTTGAAAGATGGGGTCAATCCCTCATTCATTATCAGTGGTGA
GCATCAGGCTGACAAAACACCTCCACCCAGAACTCCCCCTGGCTCTGCAAGCTGTGCTAGCTCTTTGTCAATCACTG
AAAAGAAAGCCCAACCATCCTATCCTAGAATTGCTCCTGAGATGGGGAGGTAAGCGATATGCAGGTTTAATCAAGGG
GCTGGGGAAAAGGCGTACCAGCACTCGTTCTTCCAAGAAATGATCAGAAGAGCCGCTGTTGAGGCCAGGTGCAGCTA
GAGCTCTGCCATTTTTCGGGTTTTCATCAGGGAAAGTCTCTCTGTTCTAGGGCAGTGTTTGGACAAGCACTCACCTC
ACACACACACACTTCTGAGAGAGCAGGAAAGGAAATCCAAAAGAGGCTTGAGTCTTTGAATATAAAAGCTGGTAAAC
ACACACACACACACACACACACACACACACACACTCCTTAGAAGTTTCACTGTTTATCAACTAGGAATACATTTTAA
ACAATAGTTCTTCAGAGAGGATGGGAAATTAAGTCAAGGTCATAAATCAAAATCAGAGAGCTGCCGTAAAGGAGCTT
AAGAAAAAGTTAGGCATGTGCTGGGGGAAATAGCATGTTGATTGGATCATTTAAAATTTCTCAATGAGCACATTTCC
TGCCAAACCTAATTGGGAGAAAGGATCGCCAGGGAGAAAGCAAAGGATTCTCAGTACCTTCCATTTAGATCCTCAAT
GTCTTTAATGAAGAGGCCTCCTTGGTGCTTGCACATGTTCTTACATGCCTTCAGGCGGCTCTTATTCTTAAATAAGA
TGTAATCCTTGCCAGTGCTCTTATTTCGAACAAAATTGATTCCTTCCTTGAGATTGGCAGCTTCGGCAGGTGAGAGG
CACAACAGGATCTCCGTCGTTTGTTCGATG

| | |
|---|---|
| SEQ ID NO: 15 | CMAH cDNA Sequence |

ATGAGCAGCATCGAACAAACGACGGAGATCCTGTTGTGCCTCTCACCTGCCGAAGCTGCCAATCTCAAGGAAGGAAT
CAATTTTGTTCGAAATAAGAGCACTGGCAAGGACTACATCTTATTTAAGAATAAGAGCCGCCTGAAGGCATGTAAGA
ACATGTGCAAGCACCAAGGAGGCCTCTTCATTAAAGACATTGAGGATCTAAATGGAAGGTCTGTTAAATGCACAAAA
CACAACTGGAAGTTAGATGTAAGCAGCATGAAGTATATCAATCCTCCTGGAAGCTTCTGTCAAGACGAACTGGTTGT
AGAAAAGGATGAAGAAAATGGAGTTTTGCTTCTAGAACTAAATCCTCCTAACCCGTGGGATTCAGAACCCAGATCTC
CTGAAGATTTGGCTTTTGGGGAAGTGCAGATCACGTACCTTACTCACGCCTGCATGGACCTCAAGCTGGGAGACAAG
AGGATGGTGTTCGATCCTTGGTTAATCGGTCCTGCTTTTGCGCGAGGATGGTGGTTACTACACGAGCCTCCATCTGA
TTGGCTGGAGAGGCTGAGCCTTGCAGATTTAATTTACATCAGTCACATGCACTCAGACCACCTGAGTTACCCAACAC
TGAAGAAGCTTGCTGAGAGAAGACCAGATGTTCCCATTTATGTTGGCAACACGGAAAGACCTGTATTTTGGAATCTG
AATCAGAGTGGCGTCCAGTTGACTAATATCAATGTAGTGCCATTTGGAATATGGCAGCAGGTAGACAAAAATCTTCG
ATTCATGATCTTGATGGATGGCGTTCATCCTGAGATGGACACCTGCATTATTGTGGAATACAAAGGTCATAAAATAC
TCCATACAGTGGATTGCACCAGACCCAATGGAGGAAGGCTGCCTATGAAGGTTGCATTAATGATGAGTGATTTTGCT
GGAGGAGCTTCAGGCTTTCCAATGACTTTCAGTGGTGGAAAATTTACTGAGGAATGGAAAGCCCAATTCATTAAAAC
AGAAAGGAAGAAACTCCTGAACTACAAGGCTCGGCTGGTGAAGGACCTACAACCCAGAATTTACTGCCCCTTTGCTG
GGTATTTCGTGGAATCCCACCCAGCAGACAAGTATATTAAGGAAACAAACATCAAAAATGACCCAAATGAACTCAAC
AATCTTATCAAGAAGAATTCTGAGGTGGTAACCTGGACCCCAAGACCTGGAGCCACTCTTGATCTGGGTAGGATGCT
AAAGGACCCAACAGACAGCAAGGGCATCGTAGAGCCTCCAGAAGGGACTAAGATTTACAAGGATTCCTGGGATTTTG
GCCCATATTTGAATATCTTGAATGCTGCTATAGGAGATGAAATATTTCGTCACTCATCCTGGATAAAAGAATACTTC
ACTTGGGCTGGATTTAAGGATTATAACCTGGTGGTCAGGATGATTGAGACAGATGAGGACTTCAGCCCTTTGCCTGG
AGGATATGACTATTTGGTTGACTTTCTGGATTTATCCTTTCCAAAAGAAAGACCAAGTCGGGAACATCCATATGAGG
AAATTCGGAGCCGGGTTGATGTCATCAGACACGTGGTAAAGAATGGTCTGCTCTGGGATGACTTGTACATAGGATTC
CAAACCCGGCTTCAGCGGGATCCTGATATATACCATCATCTGTTTTGGAATCATTTTCAAATAAAACTCCCCCTCAC
ACCACCTGACTGGAAGTCCTTCCTGATGTGCTCTGGGTAG

| | |
|---|---|
| SEQ ID NO: 16 | CMAH Protein Sequence |

MSSIEQTTEILLCLSPAEAANLKEGINFVRNKSTGKDYILFKNKSRLKACKNMCKHQGGLFIKDIEDLNGRSVKCTK
HNWKLDVSSMKYINPPGSFCQDELVVEKDEENGVLLLELNPPNPWDSEPRSPEDLAFGEVQITYLTHACMDLKLGDK
RMVFDPWLIGPAFARGWWLLHEPPSDWLERLSLADLIYISHMHSDHLSYPTLKKLAERRPDVPIYVGNTERPVFWNL
NQSGVQLTNINVVPFGIWQQVDKNLRFMILMDGVHPEMDTCIIVEYKGHKILHTVDCTRPNGGRLPMKVALMMSDFA
GGASGFPMTFSGGKFTEEWKAQFIKTERKKLLNYKARLVKDLQPRIYCPFAGYFVESHPADKYIKETNIKNDPNELN
NLIKKNSEVVTWTPRPGATLDLGRMLKDPTDSKGIVEPPEGTKIYKDSWDFGPYLNILNAAIGDEIFRHSSWIKEYF
TWAGFKDYNLVVRMIETDEDFSPLPGGYDYLVDFLDLSFPKERPSREHPYEEIRSRVDVIRHVVKNGLLWDDLYIGF
QTRLQRDPDIYHHLFWNHFQIKLPLTPPDWKSFLMCSG

| | |
|---|---|
| SEQ ID NO: 17 | CXCL10 Genomic Sequence |

CTTATAGTAACTTTATTACCTTTTTTGTCTGAACAGTTAGTCTTTCTTAATGTTTCTAGGAGAGAACATTAGTTTTA
TTTTGAAGAGCACCCACTCAGCGTATTTGTCTTACATAACATGCAGAACATGTATCCACATTTAAAAATTTATCTCA
TTGTAGTACATACTTTTACAAGGTATTCCATAAACACTGAAAACTATAAGAAACATATACATCTAAGAATCCTACTT
TATATAGTCTTTCACTAAATAATACTATTTTCATATACATTTTCAGGTATTTCTAGCTTCTCCTGTGTATTTAGAAT
TATGTATGTAATCACCAAGAGAATATGGGCCCCTTGGAAGGAAAGCAGTAGAAGCCCACGGAGTAAAGATCTTTCTT
TAAAAAGCAGGTTTTATTATTGTTTTAAATACCTCTTGGTTATTTGAGATTCTAAGAACTTCGATTAAGTCCCAAAG
TGGAATGATCCCTTAATAACCAGACGATAGGAAAGGTGAGGAAAGTGTCAGTAGCAGGGCCAGGACTTGGCACATTC
ACTAAGAATGTAGCACCTCAGTGTAGCTTATAGTATAGTGCCTGGGCAGAGTTACTGCTCAACAGCTCGGGATGATG
AACCATCTGCTGCCCTGCAAGTGTGGGAGCAGCTAACTTGGTGACTGCAATCCATGGACAGTTAGGGCTTGATGTAT
GGTGTATGTAGAGAGATGATGGCAGAGGTAGATTCTCTCCGGCCCATCCTTATCAGTAGTGCCGTGATTATGCTTCT
CTCTGTGTTCGAGGAGATCTTTTAGACCTGTAAGAAGAGAGGGAGAGTGTGAAAGACTCTGGTTTCAGTCTGAGTTC
TGCTTGGAACACACTGAATTCATAGATAATCCCAAGTTCTCAGGTGAAGTGTGGTGAGATTTCCTGCTACACAATCA
TTGTGTGTTACAGGGGATCCTTTTTAAAAAAGGCCAGGAAAGGCTTGTGGGAAATTTGGTATCTTTGCTTGGATAGT
TATAACTCTGCCTCAAGGTTGAAATGACCTATTGACACTTCTAGATAGGGAATCAGGTGACTTGATATACCACATAA
GATGACATCTCAGTATATAAGCACATGAAGGTAATGGCACAGTGGTGGTAACACTCTTTTAAGCCAAAGATTCCCAG
GAAGGCCCAATGCAAATATTTCTAACTTCCCAAAATTGACATTTCTTAAAGAGAAATACTTCTGCAAGCAGTAGCAA
ACCTACCTTTCTTTGCTAATTGCTTTCAGTAAATTCTTGATGGTCTTAGACTCTGGATTCAGACATCTTTTCTCCCC
ATTCTTTTTCATTGTGGCA

| | |
|---|---|
| SEQ ID NO: 18 | CXCL10 cDNA Sequence |

ACGCGGGGGAGACACTCTTCAACTGCTCATTCTGAGCCTACTGCAGAAGAATCTTCAGCTGCAGCACCATGAACCAA
AGTGCTGTTCTTATTTTCTGCCTTATTCTTCTGACTCTGAGTGGAACTCAAGGAATACCTCTCTCCAGAACTGTTCG
CTGTACCTGCATCAAGATCAGTGACAGACCTGTTAATCCGAGGTCCTTAGAAAAACTTGAAATGATTCCTGCAAGTC
AATCTTGCCCACATGTTGAGATCATTGCCACAATGAAAAAGAATGGGGAGAAAAGATGTCTGAATCCAGAGTCTAAG
ACCATCAAGAATTTACTGAAAGCAATTAGCAAAGAAAGGTCTAAAAGATCTCCTCGAACACAGAGAGAAGCATAATC
ACGGCACTACTGATAAGGATGGGCCGGAGAGAATCTACCTCTGCCATCATCTCTCTACATACACCATACATCAAGCC
CTAACTGTCCATGGATTGCAGTCACCAAGTTAGCTGCTCCCACACTTGCAGGGCAGCAGATGGTTCATCATCCCGAG
CTGTTGAGCAGTAACTCTGCCCAGGCACTATACTATAAGCTACACTGAGGTGCTACATTCTTAGTGAATGTGCCAAG
TCCTGGCCCTGCTACTGACACTTTCCTCACCTTTCCTATCGTCTGGTTATTAAGGGATCATTCCACTTTGGGACTTA
ATCGAAGTTCTTAGAATCTCAAATAACCAAGAGGTATTTAAAACAATAATAAAACCTGCTTTTTAAAGAAAGATCTT
TACTCCGTGGGCTTCTACTGCTTTCCTTCCAAGGGGCCCATATTCTCTTGGTGATTACATACATAATTCTAAATACA
CAGGAGAAGCTAGAAATCCCTGAAAATGTATATGAAAATAGTATTATTTAGTGAAAGACTATATAAAGTAGGATTCT
TAGATGTATATGTTTCTTATAGTTTTCAGTGTTTATGGAATACCTTGTAAAAGTATGTACTACAATGAGATAAATTT
TTAAATGTGGATACATGTTCTGCATGTTATGTAAGACAAATACGCTGAGTGGGTGCTCTTCAAAATAAAACTAATGT
TCTCTCCTAGAAACATTAAGAAAGACTAACTGTTCAGACAAAAAAGGTAATAAAGTTACTATAAGCCAAAAAAAAAA
AAAAAAAAAAAAAAAAAA

| | |
|---|---|
| SEQ ID NO: 19 | CXCL10 Protein Sequence |

MNQSAVLIFCLILLTLSGTQGIPLSRTVRCTCIKISDRPVNPRSLEKLEMIPASQSCPHVEIIATMKKNGEKRCLNP
ESKTIKNLLKAISKERSKRSPRTQREA

| | |
|---|---|
| SEQ ID NO: 20 | CIITA Genomic Sequence |

GCAGTGGACAGTGCGCCACCATGGAGTTGGGGCCTCTGGAGGGTGGGTACTTGGAGCTTCTCAACAGCAGTGCCGAC
CCTCTGCAGCTCTACCACCTCTATGACCGGATGGACCTGGCTGGAGAAGAAGAGATCGAGCTCTGCTCAGGTGGGCC
CTCCTCCCTCTGGCCCTTTTCAAGTCCTTCCCCAGCCCTCTGCCTGCCATGGAGCGCTGCTCAGCACCACGGACAGC
TCCAGAGCCCGCCCCCCGGGGGCGGGCTCCTCGTGGGGACATCTCCCAGCCTGCCCGGCTACCCCCTCCTTCCCCAC
CAGCCCTCTTTCCTGGCTCTTTCCTGCTTCATCCAAGTGGCTTTTCCTCCCAGAACCTGACACGGACACCATCAACT
GCGAACAGTTCAGCAGGCTGTTGTGCGACATGGAAGCAGATGAAGAAACCAGGGAAACTTACGCCAGTATCGGTGAG
GAAGCATTCTGAGCCAGAAAAAGGACAAGCGAGGGGAAGAGGCTTCTTTTCTCTTTGGTTAATCTCACCCACTCACC
AGGAGCCAGCAGGCCCTACCTCAGAAATCTGGGCCAGGGGGATGGGGAGTGAGGGCTGGAAGGACGGAGAATCAGGG
AAGAAGAGAGATGGAGAAGGGGAGGGAAATAGACCCCTTCACCAATGAACACCAGGCAATTAAGTCGCACTTTTACA
GAGCTCCCATTGTGGCTCAGTGGTAACAACCCTGACGAGTAACCACGAGGGTGTGGGTTCGATCCCTGGCATCGCTC
AGTGGGGTTAAGGATCTGCTATTGCCCTGAACTGTGGTGTAGGTCGCAGGTGTGGCCTGGATCCTACATTGCCGTGG
CTGTGGTATAGACCAGCAGCTGTAGCTCTGATTTGACCCCTGGCCCAGGGACTTCCACACATTTTACATGGGGCCCT
TTAAAAAAGACAAATCTCACTTTTACATCCTCTGCCTCTATTTCTACATCTTTTTCTATTAGTTGCTCTTCTTTCCT
TCCTTCCCACAAAGCCTATGTCATACACCGCTCCCTCTCTCCCAAGCTCCCAAGCTAAACTACTCTAGTATTTGTAG
TAACTACCATTTGGGGAGCATTTGCAGCCTGCTAATCGCTGTGCGTGTCTTATCACATTGAATCCTTACAAAGACAA
AGGAAGTAGATATTCTTAGTATTTTCACTTTACAGATGAGGCAACTGAGGTTTAGCGAGATAAAGCAATTCACCCAT
GTCTGCGTTAGAGACAGTAATGGGCATGTCTGAAATTCTAACTGAGGTCTTATTTTTAACCACAAAAACCAAAGTAC
CTAGGGTGGGGAGGTTTGCTAAGGCTTAATCTAAGAGGCTGGTTTGCAGCTTTATTGTTTTTTTTTTTCTTTTTAGG
GCCACACCTGCAGCATATGGACGTTCCCAGGCTAGGGGTCAAATCAGAGCTGCAGCAGCCAGCCTGCACCACAGCTC
ATGGCAACACCAGATCCTTAACCCACAGGGCGAGCCCAGGGATCGAAGTCGCATCCTCATGGATACTAGTCGGGTTT
ACTGCTGCCGAGCCACAGTGGGAATTCCTTGTTTGTAGCTTTAAAAAGAGCGACACGGATCCCACGTTGCTGTGGCT
GTGGCATAGGCTGGCAGCTGCAGCTCTGATTTGACCGCTAGCCTAGGAACCCCCATATGATACAGGTATGGCCCTAA
AAAGACAAAAAAAAATTAAGAGCTGCATTATAAACTACAACAGAAAAAAATGTTAAAGACTACATATGTACAACTGA
ATCATTCTGCTCTACACTTGAAACTAAAACAATATTGTAAATCAACTATACTTCAATTTTTAAAAAGAGCCTCAGCT
TTCAGTCAAGGGTAGAACTCTTTGGGGAGAAAAGTTTCTGTTCTGTTGTGTTTTTTGCGGGGTAGGATGGGGTAAAG
GCTCTCTCCTTACCAGGGACATCGCTCTCTTATACAGAGGCTTTGTTCAAATATAAAAAGATGCTCCTTCTTCTGGA
GGATGGAGCCCCCATTAAGAAGTAACAGCTTGGGAGTTCCCGTCGTGGCGCAGTGGTTAACAAATCCGACTAGGAAC
CATGAGGTTGCGGGTTCCGTCCCTGCCCTTGCTCAGTGGGTTAACGATCCGGCGTTGCCGTGAGCTGTGGTGTAGGT
TGCAGATGCGGCTCGGATCCCATGTTGCTGTGGCTCTGGCATAGGCCAGAGGCTACAGCTCCGATTTGACCCCTAGC
CTGGGTACCTCCATATGCCACGGGAGCGGCCCAAGAAATAGCAAAAAGACAAAAAGNCCAAAAAAAAAAAAAAAAAA
AAAAAAGTAACAGCTTGGCTATCAAAGTGCAGTCTGGATTTCTGCCCCTTTTGCCCTCTTGGCTAGGCCCCCTTGTA
CAGTGAACAACCTTCACAACTGTTTTTAGTGGCCCTTTTCCTGGCAACCCAGGAACGACATCCCTTAGGAGGTCTGG
CATAAATGTGGCCAGTCTTTCCACAGCACAGAGGGCAGAAAATGGAGAGGAACAGTAACCGTACGTGTCTCAAAAAT
TGCAGAACTGAGAGCCTGCCTGTTTCCTTTCCTTTCTGGGAATTTACTTGCTGGAAGGAGAAATATTTGGGCCTGAG
GGTATTCACAGTTCCTCACAACTGGAGGTAGTAACGAAGGATTTGGGCTTTTTCCCAAGTCACTTAGGAGGGGGGAC
TTTTTCCCTTTAGAGGCATCTACACAGGAAGCGGGAGCATGTGGAGGAGGCAGCTTCGCCCAAGTCCGTTCCTCAAA
CCTGTGCTCCTAGAATCTCTGGCCAGGTAGTCATTTGAGCAACCTTGGCTTCTATAGAGATAAACTGGGAATAATAA
TCCCACCTGCCTCGTGGAATGACTGTTTCTGTGCATAAAGTGATTAGAACAGGATTTTGCAAAGAGTGAGCACTCAG
TAAGTGTCAGGTTCCACCCCACCACGACCACCAACACCGTCATGTCATCATTATCATGTTTGTCATCGTCTTCATCA
CCATTATATCTTCCCTCCATTTCCTCAGCACAGAAGCCTTGTATGGCTCCCCACTGCCTATAAAATCAAGTCCAAAC
TTTCCCCGACATGAAACTTTTAACTGCAGATACCAGTCTCTAAGAGTTTCCCAAACGGCTTTCCTCCCTCTGTCCCC
ACCACCCAGAAAGCCCTCCTCTTTCCTCCTCGCAGACTCTGCCCCATCTTTCTTTCTTTCTTTCTTTTTTTTTTTTT
TTTTTTTTTTTTTTGGTCTTTTTGCCTTTTCTAGGGCCGCTCCCACGGCATATGGAGGTTCCCAGGCTAGGGGTCTA
ATCAGAGCTGTAGCTGCCAGCCTACACCACAGCCACAGCAACACGGGATCTTTAACCCACTGAGCGAGGTCAGGGAT
CGAACCCGCAACCTCATGGTTCCTAGTCGGATTCATTAACCATTGCGCCTCAATGGGAACTCCTGCCCCATTTTTCA
AAGTCTAGCTCCAGGACGTCCTTCTCTGGGACATCCTCCCTGATTGCCCCATCCCACTTTACACCCTCTCCTGTATC
TCCTGCCATGATAACTGTCATCCTGTTGGCTCCAAGCCAGGTTCCACTTCATACAGTTTACAACTGCTTACTGAGTG
TCAGCTGTGTACTGACTACTGTGTTGACTGCTGGAAAGGCAAAGCCTATACGCCTCACCATCCATCCCTGAATTGTA
GGCATTACTTGTTCTCATCACGTAGAGGAGGAAACGGGGACCTAACTGGCCTAAGTTTGTACGGCTAGTAGGGTGAG
TGAGGGGTAGAGCTGAAATTTAAACTCAAACCCAAGACAGCTCTACTATACTACTGGCACTACTTTATAGTACTAGA
TACACATCATCCCTCTGATTAGGTTAAGAGCCCCTGAAGAGTCAGTGATCATTCATTCAGCAAACCTTTATGGACCC
CCATTGTGGGCCAGGTCTGGACAGTCATGACTGCCCAATGCCCAGCCCAAGGCCAGGCACACAATAAGCGTGAGGTG
AAAACTCACTGATTGACGGCACTTTTCCTTGTCTGGACAGCGGAACTGGACCAGTATGTTTTTCAAGACTCTCAGCT
GGAGGGCCTGGGCAAAGACATTTTCAGTAAGTTGGGGGGTGGGGGGTTCTTGGTTCAGCCTGCATTTCCTTCCTTGT
TCCTTAGGGGGCATGGAAATACCCAGAGGCCACCCTTCAATGAGAAGTCACGTTCCCTTCCCAGTGTAGGGACAATG
AGGGCTCATCTCGGACATCCTCTGACTGTGTGTCTTGGTGTCTTTGGTTTTTTCTCTGAAGTTGAGCACATAGGATT
GGAAGAAATGATCAGTGAGAGCGTGGAGGTGCTGGAGGACTCAGGGCGGAAAAGTCAGAAAAGATGTGAGTGAGCGT
GTTTCCCCCCCGCCCCCTGCCATCCAACCTCTCCTGGCTTCATTCCTGGCCCTGCCCTGGCTCTAAAACCTCCCAGT
CGCATTCCTTGTTAAGCCTTGCCTGCTCTGACCTGGCTTTGGGTGTCCCCCCACCTCTCCTCTCACCACTGCTCCCT
CGAGACCCAGAGAGGAAGCAAGTGGCCCAGCAGCAGATGGTCCCTCTCCTGGTGGGTCTCTGTTTTTGACTGTCATT
TCCAAAAGACCTCTGGGCTCTGGCTTCTCTTTCATCCTTAGTTGTCACCCCTGTATTTAAGGGAGGTCTCTTCAAGG
ACAGTCTTTCCCCAGCAAGATCTGGGTTTGAATTCCAGATCTGCTATTTAAGGTCTGTGTGACCTTGGGCAAATAAT
TACACCTCTCTGAGCCTCCTAGTCAGTCTGCCTGCCTCCTCTGTCTGTCCTCACCTGGCAGCCAACATGGGCTTTTG
AATGCAAATTCAATCATTTGGCTGGCCTGCAGACCCTCCAATGGCTCAAAATACATACCACAAGGATCTGTAGGATC
TGGCCCTTCCCCCTCTCCAAATTCACGAATGTGAGTCACTATGCTCCATCCAGCCACACTGGCTTCTTTCCATTCCT
GTAACTCTTGTACCCTTTCCAGCCTCAGGGCCTTTGCACTTGCTGTTGGCCCTGTCTGGAATGCCCTTCCCCCGTTT
CTTCCCATAGTGGCGCCTCCGAATCTTGTAGGTCTTGGCCAACATGTTGCCTCCTCCCGAAGGCCTTCTTCCATCAA
CTTTTCCACATAAATTAACCTTACTTACTTTCACCTTGTTTGTGTCTCTCCAGCATCACAGCCCTTGTCACAATCTG
GACTTGTTTTAGGTATTGGCTTTTGCTTAGTTCCCCCACCATGGGGACAGGGACCTTGTCTTTCTTATGTAATCACT
ACCTTCCCCAGCACCTGGTACATGCCTGGCATGCGGGAGCATCTCCATAAATATCCACTGAATGGAAATTTCCAGGA
GTTCCCATCGTGGTGCAGCAGAAAGGAATCTGACGAGTATCCATGAGGATTTGGGTTCAATCCCTGGCCTCGGTCAG
TGGGTCCAGAAACCAGCACTGCCGTGAGCTGTGGTATAAGTCGAAGATGAGGCTCAGATCCCGTGCTGCTGAGGCCT
TGGTGGAGGCCGGCAGCAGCCGATTTGACCCCTAGCCTGGGAATTTCCACATGCCTCAGGTGCAGCCCTAAAGAGCA
AAAAAAAAAAAGAAAAAAAAATTTCCACAAAATGGGCATCACAGCTAATTGAATGCTTACTCTAGGCCAAACCATGT
GTAAGCCCTGAACCTATTTAATTTGAACAGGTAAACAGATGCATGGCATAAAAATTCAAAAGGTGCGAAGAACAGTC
AGTAAAAAAAAAAAAAAAGAAAAAAGAGCTCCTTCCCACTCGTTTCCCAGTCTTTCATTTTCCCTCTCTGAAGACAA
TCTATGCTGCCAGTTTCCTTTTTGTCTTATATTTTGCCTAAAAGCCAGCTCTTTAAAACAATGTTGCCCCACAAGTG
GCATTTCACCCACCGTCTCGGGCACCTGGCTTTCTTCGTTTACCACGTCAGGACGGCGATTTCCACACCACGATGGA
AAACACGTGGTCCTCCCGCCCAGGAATTTCCCTTTCCTTTCCTTCTTTTTTTCCTTCCTTCCCCCTTTCTTCTTTCT
TTTCATTTCATAAGCATTTTCCCCCAATATTTTACCATGTGGTGTAGGGTGCAGACTACAAAATTTCTGTCTTTTTT
TGCGTGTCTTTTAGACCCCAGGCTAGGGGTTGAGTCCGAGTGTAGGTGCCGGCCTACACCACAGCCACAGCAATGCA
GAGTCTGAGCCTCGTCTGCAACCTACACCACAGCTCACAGCAATGCCAGATCCTTAACCCGCTGAGCGAGGCCAGGG
AGCGAACCTGCGTCCTCATGGATGCTAGTCGGGTTCGTTAACCCCTGAGCCACAACGGGAACTCCGAAAAATTTCAG
CATATAGTAGAGGTGACAGAATTGTACTACAAGCAACCACATACCCACTGCTGACAACCTACCATCAGTGTTGGGCT
ATATTTGCCTTAACACATCTCTATCCATCTGTCCATCCCTCTATCATCCACCCATCCATCCATTTTCCAGGGGAACG
TGTCAAAGGACGTTGCAGACGCCAGTACTGCCCACACATCCTTCCACATCCTTGTTATTTTTAGGGCTGCATGGTAT
TTCACTGGGGGATGAATCATCGTTTGTTTCATCAGCCCCTCGCTAAGGACACAGCTGGGTTTTTCTCTGTTGATGTG
TGCCGTGCTTGATATGCACTCACTGATTTCCAGTGCATTCCTGCAAAATGGGAATCAACACCCCTGTTTCACAGATG
AGAGAACAAAGGCTCAGAGAGGCTGTGTAGCAGAGACAACACGGCCAGGAAGGGCCCAAAAGCAGGTGGTTTGTCTT
TGTTTTTTTTGTTTTTTTTGGTGGGAGGTTGTTTTTGTTTCTGTAATGGCTGCACCCATGGCATACGTTTCCAGGGC
AGGGATTGAATCTGAGCTGCAGCTGTGGCAATGCCGGATCCTTTCACCCACTGCACCAGGCCAGAGATGGAACCTGT
GCCTTCACAGCGACTCGGGCTGCTACAGTCAGGTTCTTAACCCACTGTGCCAGGGTGGGATCTCCCACAGATGTTTT
TTTCATTTTTATTATTATTATTTTTAAACTCAAACTCTTCCTGTGTCTCTTCTATGGTTCTGCCTCTTCCAGTGCCT
CACTGCCCTGGGTGCTTCAAGATGGGGTTTGGGCTCAAGCAAAAGAGTGGGGGCAGAAATGGTCGGAGGAAGAGGAG
GGAAAGGGACCCCCCAGGCCACTTCCCAGCCATTTAAGGCAAGGCCACAAGGCCTAACTGGGGTCCACAGGCCCGTC
CTGGCTGGGTCTGATGACCGTGTGTTCTCTCTGAAGCTTTCCCGGAGGAGCTGCCTGCGGATCTGAAGCACAGGAAG
CTAGGTGAGCAGGGCGGGTGCATCCAGGGAGACTGCCAGGCAGGGAAGCTGGGGTCTCCTCAGGTGTGCATATAAAC
TAGCATTTAAAAGCTGAGGCTCAGAGAGGTGAAGCCACTTGTTCAACATCACACAGCAAGTGAGAGTTGGAGTTGGG
ATTCAGACTAAGATCATGAATCCACAGTGCGTGCTCTGCAGTTCAAGGACTGTTGGGAGATTCACCTCTACCCACAA
AACCTATTTTGAACTCTGAGTCAGAGCTGAGGACCCCCCCACCCCACCTTGTTCCACTGCCCCTCCAGGCCACAGCT
CTCCTTTCGGAAGGCAGCGTCACCTCTGGTCAGCTGGTTACCCGGCGGTTCCCCCCTCCCATGCCTCAATGAGCCTC
TTCCCCATGCCTCCATCCCCCCCCCACCAGATGCTTCCTCCCCTCCCTTCCTCCCTCCTCCCTGATTCGGTTGTTAT
TGCAAAGGTGGGGAGGCCAGCTCCCCTGTGAGAAAGAGACTGAGAAATGAAAGCCTCATAGTCTGATGGAGGAAGCC
TGGTCTCTACTCCCAGGTCTAATCTGATGGAGAAGACAGGGACCCCAACCAGGAGGACCCCAGCGTGATGGAGACCC
CCAATCTGATAGGGGAGGCGAGTCTCCGCCCTCCTGAGCTCCTGATTCAATGGAGGAGATAAACTCGTGCCCCAGGG
AGACAGCAAGTGCTCGAGGTCCCTGGAGGCTATAGAAGGTGGTAGGGGCCTGGGCTAACACCCTCTTCTTAGGTGTG
TCCCGCCTGCGCCCGGCTCTCCAAGGCAGGAAGTGCTCAGGGAGGAAGCCGGGGGTGGGGGCTGTGTGACACAGCAC
AGTTGCTGCTCAGACCAGCTTCACCCAGGACTGAGAAGAGGACAGGAATTCCCTTCCACTGCCAGCAGAGAGTTCCA
CTCTGCTCCCTGAGCACTCCCCACCCTGGGAAGGACCCTCAGGGCACCCACCCAGATCTTACCAAGCCTCTGACACG
GCCCCCTTTCTCATAGCCGAGCCCCTCGCCATGCCCATGGTGACTGGCACTTTCCTGGTGGGGCCAGTGAGCGACTC
CTCAGCTCGACCCTGCCCATCACCTCCTGCTCTGTTCAACAAGGAATCAACACCCAGCCAGGCCCAGCTGGAGGACG
CTGTCCCAATGCCGGGTAGGTTAGGGGCTTGGAGGGGCAGGGCTTCCCCTTCCCGCCTCCCCGCAGGTGCCTGAGGA
GTGGCTACTTCAGGAGCCACAAGGGACAGGAACTGCTCCCCCTACTACTGTCACCCACTTCCATCCCAGCCAGTCCT
ACCCCCCAGGGTCCCCCTCGACTCCGTCTGTGCCAGAGAATGTGCCCTGGGCATCACAGCAGGGAATCCCTGCCAAC
CAGGGAATTCACTGCCAGCCCTATGCTAGTTCGCTTGCTTTCCTCAGCAGTGAACCGTGCACCCTCTCTGGGCCAGC
TGCTCTGCTGGGTGCCAGCAACACTGTGCTGGGCCAGCAGACAAAGCTTTTCAATCTCCTCCAGGCTCTCTCGATTA
GAGTCCTTGAGAAGGGAGTCAGATGTTAATTAAGATGCTCAAGTGCTGGGAGTTTGGAGTTAATAGATGCAAACTAT
TGCCTTCCTGCGTGGATAAGCAATGAGATCCTGCCGCATAGCACAGGGAACTATATATCTAGTCAGTCACTTGTGGT
GGGACATGGTTAAGGATGATGTGAGAAAAAGAATGCATACATATGTACAGCTGGGCCACTCTGCAGTACAGTAGAAA
TTGACAGAACACTGTAAATCAACTATAATGGAAAAAAATAAAATCTTTCAAAAAAAAAAAAAACAAAAAACAAAAAA
GATGCTAACGGAGAACCCTACCTTACCATCTTGGTCTCTTGCAGCGCCCCCTTCAGGTTCCTTGTTGAGCTGCCTGA
GTGTCCCTGCTGGACCTATTCAGATCATCCCCACGCTCTCCACCCTGCCCCAGGGGCTCTGGCACATCTCAGGGGCC
GGGACAGGGGTCTCCAGTATACTCATCTACCAAGGTGAGCGTGGGAAGCCAGGCTCCCCACCCCCTCTGCCTGTGAC
CTGACTATTCCCTGACGCCATCCTTTTCCCACCCCAGGCATTTAGTGCTTACAGCCCAGCACCTTCTCAGGATCCTC
CGTCCCCATTTCCCCAAACTCAAAAGAGAGGAGCAAAGCTCCCGCGTGTTCTAAGCGACCCAAGTGCCTAAGTGACC
TTTTTTGGTCACTTTTCTCCACGAAGCCTTAGTTTCTCCCTTTTAAGAAAAATAACTTCATTATACTTTAAAATCCA
AATATTTATGTATGCTCATTAAGAAACCAAAAAATAAGACCTACTTACAAGAGTCACGGAGTCTCCCCATCGCTCTT
TTTAGTATACCGTTGTGAATAGTTTGGTATGGATCCTTGCACAGCTTTCTCAAAGTTGTCTTGTTTCCGGGTCTGTA
AGAAGGTCCTTGCTGACCTGCCACATTGGAGGGTTTTAAATTGTCCAAGGGAAGGCACGTTGGGCTCTCAGGGATGG
GAGAGAGAATGAGGCTAAGGAGATATTTCCACTCAACTCAAGAGCATCCTTTGAGGACTTTCCACTGTGGCACAGCA
GAAATGAATCCAACTAGTATCCATGAGGATGTGGGTTCAATCCTTGGCCTCCCTCACTGGGTTAAGGATCCTGTGAT
GCTGTGAGCTGCGGTGTAGGTCGCAGACACGGTTCGGATCCTGCGATACTGTGGCTGTGGTGTAGGCCGGCGGCCGT
AGCTCCGAATCAACCCCTAGCCTGGGAACCTCCATGTGCCGCGGGCATGGCCCTAAAAAGCAAAAAAAAAAAAAAAC
AGTAGAACTGCGCTGCCGCTTGGCTCACAGTCTCCGGTTTTACGGGAATGGGGTTAGTTTCTGGGTGGTCTATGGCC
AATTGTCTTGCCTGACCCGTGCTTGGTCCGCCTCGCGCGGGGACTTTCTGGGTGGCGCACACACCTCTCAGCCAAGA
TGGATTCCAGCGCCAAGGATCCTGGGAAGTTGGTGGTCTCCTCCCTCCCACAGGCCCCTCCCACGGGCCCCTCCCAC
ATCCTCCCGGTTAGTCTTCAGGGCAGCAGCACATTCCTCACGGGGCCTCCTGTTTCGAGACACCTCCTGCTAGTGGT
TGTTATCCTGCCTGGCCGAGGTGGACAGTTTCGGCCAGTCGTCCCCTAACAGAAGCACTTGCCCTGCTCCCAAGGAG
CTGGTTGTGTCCCTTCACAGATGGGGAAATCAAGGCTCCGGGAGCTCCATGTCACTCCNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNN
NNNNACGAGAGCCAGAGCTCCAGCAGCTTCCAAGTGGCCAGGTGAGTGGTGGCAGGGTCCCTCTGCCCAGGTGCTGG
ACGTAGAAGCCCAAATCCGACTTCCCTTCATGCATTCACCCAACACTTGTTCAATCTCTCTTTTGTTGGCTCACTCA
TTCATTCATTCACTCATTCATTCACATGCTCATTGCATCTTCACATCATCTCATCACTCATTCCTCTGGTTATACCT
ACATTTAAAGCTACCTTTACCGAGGACCTGCCCCGGGGAAGCCCATGCTGGGCGTCAATATCTTTTTTTTTTTTTTT
TTGTCTTTTTTTTTGTTATTTCTTTGGGCCACTCCCGCGGCATATGGAAATTCCCAGGCTAGGGGTCTAATCGGAGC
TGTAGCCGCTAGCCTACGCCAGAGCCACAGCAACGCGGGATCCGAGCCACGTCTGCAACCTACACCACAGCTCACGG
CAACGCCGGATCGTTAACCCACTGAGCAGGGGCAGGAACCGAACCCGCAACCTCATGGTTCCTAGTCGGATTTGTTA
ACCGCTAAGCCACGACGGGAACTCCTGGGCATCAATATCTTGTTAGCGAGGCTGAGAGAGTGAATGAAGGGAGCGTG
GGTGACCGAGGGAACTAAGACAGGAGTGGGGATGAAAGGGCAGCTGACTGCTGAGTCTGACTCTGTCCCTGGTACTC
CAACACAGGAGATGTAGTAAATCAGGAAAGTCCCAACCTGACTATGGTCCCCATTTTGTGGAGGAGAAAACTGAGGC
ACAGTGGGGTATCGCACATGCTCAAGATAATACTAGTAAGTGGTGGAGCCAGGACTTAAACCAGAAACATGGATTCC
ACTATCTTAACCCTCAACACACACACACACACACCTCCCCAGAATGGTCTCCCAATCGTGAGTGAGCAAAAGAAGAA
AATCTTGGAGTGGGTAAATGATGGAGAAGATGAGGGAATGAATGAGCGAATGAGGCAGCTAATCCAGAAAGCCATCA
GGGAAGACGGGTGAATGGACGAAGAAGCTAGTGATGGTGGCCGGGCTGGCCTCTCGGCTGCCCTCCTGGTAGCCGGT
CCTGCCACTAGCATCCTCCCCTCCCCCACTCCCGCCTTTGACCTGTGCAGAGACTGTGGAGCAGTTCCACCACTCAC
TCCGGGACAGGTACCAAGCCAAGCCCGCAGGCCCGGAAGGCATCCTGGTGGAGGTGGACCTGGTGAGGGTGCGGCTG
GAGAGGAGCAGCAGCAAGAGTCAGGAGAGAGAGCTGGCCTCCCTGGACTGGGCAGAGCGGCAGCCAGCCCGAGGGGG
TCTGGCGGAGGTGCTGCTGGCCGCTAGCGACCGCCAGGGGCCACGCGAGACGCAGGTGATCGCCGTGCTCGGCAAAG
CAGGACAAGGGAAGAGTCACTGGGCCCAGGCCGTGAGCTGGGCCTGGGCTGACGGCCAGCTGCCACAGTACGACTTT
GTCTTCTGCATCCCCTGCCACTGTTTGGACCGGCCGGGGAACACCTACCGCCTGCAGGATCTGCTCTTCTCCCTGGG
CCCACAGCCCCTGCCCATGGACGACGAGGTCTTCAGTTACATCTTGAGGCGGCCGGACCGCGTTCTGCTCATCCTGG
ATGCCTTCGAGGAGCGCGAAGCCCAGGACGGCTTCGTGCACAGCGCGGGCGGACCCCTGTCCTCAGAACCCCGCTCC
CTTCGGGGGCTGCTGGCTGGGCTCCTCCAGCGCAAGCTGCTGCGAGGCTGCACCCTGCTGCTCACGGCCCGGCCCCG
GGGCCGCCTGGCCCAGAGCCTGAGCAAGGCCGACGCCCTGTTTGAGGTGGCCGGCTTCTCCGCACAGCAGGCCAAGA
CCTACATGCTGCGCTACTTTGAGTGTCGGGGGGCCCGTGAGCGCCAGAAGAGAGCCCTGGAGCTCCTCCAGGCACAG
CCGTTTCTCCTGAGTCACAGCCACAGCCCTTCCGTGTGCCGGGCCGTGTGCCGGCTCTCAGAGACCCTCCTGGAGCT
GGGCGAGGAGGCAGAGCTGCCCTCCACGCTCACCGGCCTCTACGTCGGCCTCCTAGGACCAGCGGCCCGCGAAAGCC
CCCCGGGTGCCCTGGTGGGACTGGCCAGACTGGCCTGGGAACTGGGCCGCCGTCACCACAGCAGCTTGCAGGAGGGC
CAGTTCCCATCGGCAGAGGCCAGGGCCTGGGCTGTGGCCCAAGGCTTGGTGCAGCGTGCCCCGGGGGCCCCGGGGGC
CCCTGAGCTGGCCTTCTCCAGCTTCCTCCTGCAGTGCTTCCTGGGGGCCGTGTGGCTGGCTCTGAGCAGCGAGATCA
AGGACAAGGAGCTGCCGCAGTATTTGGCATTAACCCCTAGGAAGAAGAGGCCCTATGACAACTGGCTGGAGGCTGTG
CCACGCTTTCTGGTCGGGCTGGTCTTCCAGCCTCGCGCCCGCTGCCTGGGAGCCCTGGCAGGGCTGGTGGCAGCCAC
CTTGGCGGACCGGAAGCAGAAGGTGCTCAACAGGTACCTGAAGCGGCTGCAGCCCGGGACCCTGCAGGCAGGGCGGC
TGCTGGAGCTGCTGCACTGCACGCACGAGGCCCTGGATTCTGGGCTTTGGCAGCATGTGCTGCAGGGGCTCCCGACC
CAACTCTCCTTTCTGGGCACTCGGCTCACGCCTCCGGACACCCACGTGCTGGGCAGCGCCTTGGTGGCTGCAGGCCG
AGACTTCTCCCTGGACCTCCGCAGCACTGGCATTGACCCCTCTGGACTGGGGAGCCTCGTGGGACTCAGCTGTGTCA
CCCATTTCAGGTGGGGGCCGGGGACAGGAGAGAGGGCTTCTTTGCATTGAGCACCTACTGTGGTTTTGCTGCTGTGC
CCAGTGCTGGCTCTGTGGGGTCTCATTCAGTAGGCATGGCAGCCAGATGTGGGCAGAAGTGATTCCACTCATTTGAA
GATGAGGAAGCCAAGGCTCAGAGAGGGAGAGTAGCTTGCCCGAGGTCACACAGCCAGTGAGAGGCAGCATCATTCTT
TTAACCACTGTTTGAAAGGGCCATGTTCCAGGCACTGGGCCATGTCTAGAGTCTAAGACTGATCTGGGTTCAAATTC
ATTTTCTTCTCTCCATCCCCTGATCAAGTCACCATTTTGTCATGGTTAGATTAAAACCACAGCCTCCCCTGACTTCC
CTGCCCCCGTTCTCGCCTCTTCCACTCCATTTTATTTTATTTTATTTTATTGGTTTTTAGGGCTACACCTGTGGAAT
ATGGAAGTTCCCAGGCTAGGGGTTGAATCCGAGCTATAGCTGCTGCCCTACACCACAGCCATAGCAACGCAGGATCC
TTAACCCACTGAGGGAGGTCAGGGATTGAACCACATCCTCATGGATCCTAGTCAGGTTCGTCACCACTGAGCCATGA
CAGGAACTCCCCCACTCCACTTTATTCTTAACCATCAGAGCAATCTCCCTAGTAATTGCATCTGATCATCTTTCATC
CTTGCTTACAATCTTTTAGAGGCACTCCACCTCCCTCAGGTTGAAGTCAAAGTTCCTTAATTTAAGGAATCTAAATC
CTCCTGTGATCTGTTTGATCCCTTAAGCCTTATTTCCAGAGAATCTCTCCTACCTTCCCTCTAAGCATATTTTACCA
GAGCTATAAGGTCTACACCATTGTAATGGTTCAACGGAGAATTCAGCACTGAGCTTCCTGGTAGCCAAAGCAAAAAG
GAAAAGAAAACCCAGGAGAGCTAAGAAAAAGGAGGAATTGATAAGGGCTTAAGTGGTCATGGAAGGCTTTCTAGAGA
AAGTAGGGGGTTAAGCTGAGCAAAGAAAGTACCTGAATAGGTAGGAGGTCCCTTCATGGAGTTGCCCATCCGTTATG
GTCTAGCCCGGTCACCATGCCTGGGTCTGAGGCCCTTCCTCCACAGGGCCGCCTTGAGTGACACAGTGGGGCTGTGG
GAGTCTCTACAGCAACGTGGGGAGACCAAGCTACTCCAGGCACTGGAGGAGAAATTTACCATTGAGCCTTTCAAGGC
CAAGTCCATGAAGGATGTGGAAGACCTGGGCAACCTCGTGCAGATCCAGAGGTGAGGAGGAAAGGGCACGGGAGGTG
GTCCAGGCCATGCAGGTCCATTACATTTGTCATTAGCACTTCCAGTGCCTCATCTTTGGGGGATATCCCATGTCCTC
CGCTTGGACAGTGGCCACCCAGAATCTCTCACTGTTGTCACCACCCATGCAGAACTCCCAGGATTTATCACTTGGTC
CCATTAAAAACTTGCAGTCATGTTCCCAATTTTTTTTTTTCTTTTTTAGGACCACACCTTCAGCTTATGGAAGTTCC
CAGATGAGGGGTCAAATCGGAGCTATAGCTTCTGGCCTATGCCACAGCCACAGCCACAGCAATACCAGATCCAAGCC
ACATCTGTGACCTACACCACAGCTCATGGCAATGCTTGATTCTTAACTCACTGAGTGAGGCCAGGGATCGAACCCGT
GTCCTCGTGTGTACTAGCCAGGTTTGTTACCCCTGAGTCACAATGGGAATCCCCCTAATTCTTTCTCAGCTAAAGCC
AGGGAACTATTCTCTGCTGCTAAGAGTTCACGAGCTGCCTTCTGCATCTAGTAACAGAAGTGACACTATGGCCACCT
TTCAAGGCAGCCAGGACCAGTATCATCCCCATTTTTTTGATGGCAGAGATCTAATGTCTAGTGGGTAGAGGACACTT
GACCACAGAACAACTGCCTTTCCCTCATTCCTTCATCATACATTGTTCGAGCACCTACTATGTGCTGTCTGGGATGG
GATGGGTCTCCTCTGAGGCTCTTTTCCATGAAACACACAGGAATATTAGCCTTCATAACATCCTGTTCTGAGGCTTT
TCTTTTTAAGAAGGGCATAACAAGGAGTTCCTGTGGTGGCTCAGCAGGTTAAGAACCCAGCTAGTCTCCATGAAGAC
AGGGGTTCAATCCCTGGCCTTGCTCAGTGGGTTAAGAATCTGGTGTTGTGTGAACTATGGTGTAGGTCGCAGACACA
GCTTGGGATCCCACGTTGCTGTGGCTGTGGCGTAGGCCAGCGGCTACAGCTCCAAGTCCCCCCCTAGCCTTGGAACT
TCCTTATGCCACAGGTGCAGCCTTAAAAAAAAAAGAAAAAAAAGAAAAAAAAGAAGGGACTAACCATAGCCCGGGAA
AGGCAGTCCTTCTGGGGAATTTTGGGAATGTGGCATGCATCTTAGTACATTTAGGAAGGGACTCAGCGACAGGTGAA
GGTCCCCTGACATTGCCCATTCTCTCCATCTCTCCAGGACGAGAAGCTCTTCTGAAGACATGGCTGGGGAACTCCCT
GCTGTCCGGGACCTAAAGAAGTTGGAATTTGC

| | |
|---|---|
| SEQ ID NO: 21 | CIITA cDNA Sequence |

TTTTTTCACTTCACGTTTTGGATGCTGCAGGCCGGGTAAGCAGAGATCCCAAGGCTCTGGCCCCCGGGGAAGAGGCC
CTGTCTCCGAGCCCTACCATGAACCACTTCCAGACCATCCTGACTCAGGTCCGGATGCTGCTGTCCAGCCATCGGCC
GAGTCAAGTGCAGGCGCTCCTGGACAACCTCCTGGCGGAGGAGCTTCTCTCCAGGGAGTACCACTACGCCCTGCTCC
AGGAGCCTGACGGTGAGGCTCTGGCCAGGAAGATCTCCTTGACACTGCTGGAGAAAGGAGCCCCAGACCTGGCCCTC
TTGGGGTGGGTCTGGAGTGCACTGCAGACCCCAGCAGCCGAGAAGGACCCCGGCTACCAGGAACCTGATGGCAGTGG
ACAGTGCGCCACCATGGAGTTGGGGCCTCTGGAGGGTGGGTACTTGGAGCTTCTCAACAGCAGTGCCGACCCTCTGC
AGCTCTACCACCTCTATGACCGGATGGACCTGGCTGGAGAAGAAGAGATCGAGCTCTGCTCAGAACCTGACACGGAC
ACCATCAACTGCGAACAGTTCAGCAGGCTGTTGTGCGACATGGAAGCAGATGAAGAAACCAGGGAAACTTACGCCAG
TATCGCGGAACTGGACCAGTATGTTTTTCAAGACTCTCAGCTGGAGGGCCTGGGCAAAGACATTTTCATTGAGCACA
TAGGATTGGAAGAAATGATCAGTGAGAGCGTGGAGGTGCTGGAGGACTCAGGGCGGAAAAGTCAGAAAAGATCTTTC
CCGGAGGAGCTGCCTGCGGATCTGAAGCACAGGAAGCTAGCCGAGCCCCTCGCCATGCCCATGGTGACTGGCACTTT
CCTGGTGGGGCCAGTGAGCGACTCCTCAGCTCGACCCTGCCCATCACCTCCTGCTCTGTTCAACAAGGAATCAACAC
CCAGCCAGGCCCAGCTGGAGGACGCTGTCCCAATGCCGGCGCCCCCTTCAGGTTCCTTGTTGAGCTGCCTGAGTGTC
CCTGCTGGACCTATTCAGATCATCCCCACGCTCTCCACCCTGCCCCAGGGGCTCTGGCACATCTCAGGGGCCGGGAC
AGGGGTCTCCAGTATACTCATCTACCAAGGTGAGATGACCCAGGCCAGCCAAGCACCCCCTGTCCATAGCCTCCCAA
AGTCCCCAGACCGGCCTGGCTCCACCAGTCCCTTCGCCCCGTCAGCAGCTGACCTCCCCAGCATGCCTGAACCAGCC
CTGACCTCCCGGGCAAACATGACAGAGGGCAGTGTGTCCCCCACCCAATGCTCAGGTGATCAAGAGGCCTCCAGCAG
GCTTCCCAAGTGGCCAGAGACTGTGGAGCAGTTCCACCACTCACTCCGGGACAGGTACCAAGCCAAGCCCGCAGGCC
CGGAAGGCATCCTGGTGGAGGTGGACCTGGTGAGGGTGCGGCTGGAGAGGAGCAGCAGCAAGAGTCAGGAGAGAGAG
CTGGCCTCCCTGGACTGGGCAGAGCGGCAGCCAGCCCGAGGGGGTCTGGCGGAGGTGCTGCTGGCCGCTAGCGACCG
CCAGGGGCCACGCGAGACGCAGGTGATCGCCGTGCTCGGCAAAGCAGGACAAGGGAAGAGTCACTGGGCCCAGGCCG
TGAGCTGGGCCTGGGCTGACGGCCAGCTGCCACAGTACGACTTTGTCTTCTGCATCCCCTGCCACTGTTTGGACCGG
CCGGGGAACACCTACCGCCTGCAGGATCTGCTCTTCTCCCTGGGCCCACAGCCCCTGCCCATGGACGACGAGGTCTT
CAGTTACATCTTGAGGCGGCCGGACCGCGTTCTGCTCATCCTGGATGCCTTCGAGGAGCGCGAAGCCCAGGACGGCT
TCGTGCACAGCGCGGGCGGACCCCTGTCCTCAGAACCCCGCTCCCTTCGGGGGCTGCTGGCTGGGCTCCTCCAGCGC
AAGCTGCTGCGAGGCTGCACCCTGCTGCTCACGGCCCGGCCCCGGGGCCGCCTGGCCCAGAGCCTGAGCAAGGCCGA
CGCCCTGTTTGAGGTGGCCGGCTTCTCCGCACAGCAGGCCAAGACCTACATGCTGCGCTACTTTGAGTGTCGGGGGG
CCCGTGAGCGCCAGAAGAGAGCCCTGGAGCTCCTCCAGGCACAGCCGTTTCTCCTGAGTCACAGCCACAGCCCTTCC
GTGTGCCGGGCCGTGTGCCGGCTCTCAGAGACCCTCCTGGAGCTGGGCGAGGAGGCAGAGCTGCCCTCCACGCTCAC
CGGCCTCTACGTCGGCCTCCTAGGACCAGCGGCCCGCGAAAGCCCCCCGGGTGCCCTGGTGGGACTGGCCAGACTGG
CCTGGGAACTGGGCCGCCGTCACCACAGCAGCTTGCAGGAGGGCCAGTTCCCATCGGCAGAGGCCAGGGCCTGGGCT
GTGGCCCAAGGCTTGGTGCAGCGTGCCCCGGGGGCCCCGGGGGCCCCTGAGCTGGCCTTCTCCAGCTTCCTCCTGCA
GTGCTTCCTGGGGGCCGTGTGGCTGGCTCTGAGCAGCGAGATCAAGGACAAGGAGCTGCCGCAGTATTTGGCATTAA
CCCCTAGGAAGAAGAGGCCCTATGACAACTGGCTGGAGGCTGTGCCACGCTTTCTGGTCGGGCTGGTCTTCCAGCCT
CGCGCCCGCTGCCTGGGAGCCCTGGCAGGGCTGGTGGCAGCCACCTTGGCGGACCGGAAGCAGAAGGTGCTCAACAG
GTACCTGAAGCGGCTGCAGCCCGGGACCCTGCAGGCAGGGCGGCTGCTGGAGCTGCTGCACTGCACGCACGAGGCCC
TGGATTCTGGGCTTTGGCAGCATGTGCTGCAGGGGCTCCCGACCCAACTCTCCTTTCTGGGCACTCGGCTCACGCCT
CCGGACACCCACGTGCTGGGCAGCGCCTTGGTGGCTGCAGGCCGAGACTTCTCCCTGGACCTCCGCAGCACTGGCAT
TGACCCCTCTGGACTGGGGAGCCTCGTGGGACTCAGCTGTGTCACCCATTTCAGGGCCGCCTTGAGTGACACAGTGG
GGCTGTGGGAGTCTCTACAGCAACGTGGGGAGACCAAGCTACTCCAGGCACTGGAGGAGAAATTTACCATTGAGCCT
TTCAAGGCCAAGTCCATGAAGGATGTGGAAGACCTGGGCAACCTCGTGCAGATCCAGAGGACGAGAAGCTCTTCTGA
AGACATGGCTGGGGAACTCCCTGCTGTCCGGGACCTAAAGAAGTTGGAATTTGCGCTGGGCCCTGTCTTGGGCCCCC
AGGCTTTCCCCAAACTGGTGAGGATCCTTGAGGCCTTTTCTTCCCTGCAGCATCTGGACCTGGACTCGCTGAGTGAG
AACAAGATCGGGGACGAGGGTGTCGCCCAGCTCTCAGCCACCTTCCCTCAACTGAAGGCCCTGGAGACGCTCAACTT
GTCCCAGAACAACATCTCCGACGTGGGTGCTTGCCAGCTGGCCAAGGCCCTGCCCTCGCTGGCCGCGTCCCTCCTCA
GGCTGAGCTTGTACAATAACTGCATCTGCGATGTGGGAGCCGAGAGCCTGGCGCATGTGCTTCCAGACATGGGGTCC
CTCCGGGTGCTAGATGTCCAGTACAACAAGTTCACAGCCGCCGGGGCCCAGCAGCTCGCCGCCAGCCTGAGAAAGTG
CCCTCACATGGAGACGCTGGCGATGTGGACACCCACCATCCCGTTTGGTGTCCAGGAACACCTGCAGCAGCAGGACT
CAAGGATATCCTGAGATGATCCAGGCTGCACCCGGGACAAGCACGTTCTCTGAGGACGCTGACCACGCTGGACCCTG
ACCTGATCATCTGTGGACACAGCTCTTCTTAGGCTGTGTCCCGTGAGCTTTGGCGATCTGGTGCCCAGCCCTGGTGG
CTCAGAGTCAGCCCCCACTCTGCTGGGGAAAGGACCCACGGCCTGCTCTGTGGACAGACCCCAGGCCCGGCCCCAGG
CTCCTTCGGGGCCCAGACTGATGTCAGCCTTGCTCAGCCGCTGCAGTCCTGGCAGACAGGCGGGCACCCAGTGGCAG
SYAGGGKCCACCCGGGAGCCCTGAAGCACTCCCTGCAGGACACTGCAGACAGTGGTGGCCAGGTCAGAGTGAGGGAT
GTGGCGGCCACATCACCTGCCCAGGTCCTGCTGGCCGGGGGAGAAAGCACCTCTCCACACTGCTCCCCTGGTGGGGT
AAGCTTGGCGCTCAGAAGATACCAGCCAGCACCCCCCAGCGTGTTGATTTCCCAAACGGTGACCGACGGGGTGTCCA
CGGCAGCTGCCCTCTGCCTCCGGCACCTGCGGGTTTGCACTCACTTTGTTTGCCGAGGCCAAAGCTGGGCCTGGCCA
GACACGCCRGACCTTAGCGGGGGAAGAGCCGACAGTACACTACGGGMCGAGGYRGGGTGGCGAGGGTCTGGAACCAC
ATCCGCCTTCTTGCCCTCACGTCCTGTGTCTTTTTTCACTACATTATACATGGCTTATTCAGTCTCA

| | |
|---|---|
| SEQ ID NO: 22 | CIITA Protein Sequence |

MNHFQTILTQVRMLLSSHRPSQVQALLDNLLAEELLSREYHYALLQEPDGEALARKISLTLLEKGAPDLALLGWVWS
ALQTPAAEKDPGYQEPDGSGQCATMELGPLEGGYLELLNSSADPLQLYHLYDRMDLAGEEEIELCSEPDTDTINCEQ
FSRLLCDMEADEETRETYASIAELDQYVFQDSQLEGLGKDIFIEHIGLEEMISESVEVLEDSGRKSQKRSFPEELPA
DLKHRKLAEPLAMPMVTGTFLVGPVSDSSARPCPSPPALFNKESTPSQAQLEDAVPMPAPPSGSLLSCLSVPAGPIQ
IIPTLSTLPQGLWHISGAGTGVSSILIYQGEMTQASQAPPVHSLPKSPDRPGSTSPFAPSAADLPSMPEPALTSRAN
MTEGSVSPTQCSGDQEASSRLPKWPETVEQFHHSLRDRYQAKPAGPEGILVEVDLVRVRLERSSSKSQERELASLDW
AERQPARGGLAEVLLAASDRQGPRETQVIAVLGKAGQGKSHWAQAVSWAWADGQLPQYDFVFCIPCHCLDRPGNTYR
LQDLLFSLGPQPLPMDDEVFSYILRRPDRVLLILDAFEEREAQDGFVHSAGGPLSSEPRSLRGLLAGLLQRKLLRGC
TLLLTARPRGRLAQSLSKADALFEVAGFSAQQAKTYMLRYFECRGARERQKRALELLQAQPFLLSHSHSPSVCRAVC
RLSETLLELGEEAELPSTLTGLYVGLLGPAARESPPGALVGLARLAWELGRRHHSSLQEGQFPSAEARAWAVAQGLV
QRAPGAPGAPELAFSSFLLQCFLGAVWLALSSEIKDKELPQYLALTPRKKRPYDNWLEAVPRFLVGLVFQPRARCLG
ALAGLVAATLADRKQKVLNRYLKRLQPGTLQAGRLLELLHCTHEALDSGLWQHVLQGLPTQLSFLGTRLTPPDTHVL
GSALVAAGRDFSLDLRSTGIDPSGLGSLVGLSCVTHFRAALSDTVGLWESLQQRGETKLLQALEEKFTIEPFKAKSM
KDVEDLGNLVQIQRTRSSSEDMAGELPAVRDLKKLEFALGPVLGPQAFPKLVRILEAFSSLQHLDLDSLSENKIGDE
GVAQLSATFPQLKALETLNLSQNNISDVGACQLAKALPSLAASLLRLSLYNNCICDVGAESLAHVLPDMGSLRVLDV
QYNKFTAAGAQQLAASLRKCPHMETLAMWTPTIPFGVQEHLQQQDSRIS

| | |
|---|---|
| SEQ ID NO: 23 | B4GALNT2 Genomic Sequence |

CACATGAACTGGACAGGCCCCAGGTACATAAGAAAAAGGCCCCTAGTCCAGTAGCCAATAGGATTCCTCCTTTCTGA
AAGTCACAGCGCTTTTCCTTCCTGAGCAGAGTGGGGGCGGGGGAATAAAGTTGCGGCCACAGAGTGGACTTGAGCTC
CCCCTGGAGGCCCAAACGATTATTTGCACCAACTTGTCCTGGCTTTTGGAGTTGAGCGGGAAGAATCCGAGGGTCTT
CATTCACCGTCCTGGAAGGATAGTTTTGTCAGTGGTTTTGGTCCAGGCTGCTCGGTTGTGCCTGAAAAGTCACGGCT
GAAGGGAGCGCTGTGTGACGGTTATTGTTTGTGCCTTGACTTTTGCTTCCAAATCAGCCCAAAAGAAACTCTGCTTT
TTTTTTTTCTTTTCTAGGGCCAAACCCATGGCATATGGAAGTTCCCAGGATAGGGGTCCAATCAGAGCTGTAGCCGC
CGGCCTACACCACAGCCACAGCAACGCCAGATCCAAGCCTCGTGTGGAGACTACACCACAGCTCACGGCAACGCCGG
GTACTTCACCCACTGAGCAAGGCCAGGGATCGAACCTGCAACCTCATGGTTCCTAGTCGGATTCGTTAACCACTGTA
CCACGACAGGAACTCCACCCTTTCTGTTTTGAAAGGCACACAGACAAAGAAAACAGTCGTATTTATTATTCTGGACA
CTTTGCTTCTAAGTCATAGGAAGCAACTCAGATTAGGTTAAAGAAAAATGGGGAATTATAAGGGCACTGTGTTTTAT
AAAATCCCAGGGCAGGACTGTAGCCAGAGCTCAGGAAAGAACCAGAAGGTTTTCAGAAGTCTCTCATTTCAGCTCAG
TGGTTAACACCCTCCGAGAGTTCCATTTTAACTTTGCTGTGGTGGCACAGCAGAACCCTCTCCCCAAGGAAGGTGAC
AGGAACGTCCTTAAAATGAGGAAGAACCGCATGGCCCAATCACCCTCTCTACACGTATGCACAGCCCAGACTGTACC
CAATAAGACTGCAATAAGGCTATATGTTACCATATAAAGGGGACAAAGGGGTAAAAATAATATAAAAGGCATCTCCT
CACTGTGCTCAGGGCTCAGCCTTTGGACATGAATCTGTCGAGCCAGTGCCGGCATGAATAAATACTGCTTCCTGGAA
AAAAGCCTTGGTGGGTGTCCCATCTCTGTACGTAAGTCCTACAACAGTTCCTTCCTGCTAGAGTAGAAGGTTCCAGA
TCCTGGGGCAGGGAAGAGGTTCCTAGAACCTACTGATGATAACTACAGCACATCAAAACAGTCCCTGCTGGGGGATG
TTGGAGCATGCAACAACTGCCATGAAAGTGGACAACTCTATCTCCCTGTATCAAGAGTGCATGTTTCAGGAGTTCCC
TAGTGGCTCAGAGGGTTAAGAATCTAACTAATATCTATGAGGATGCAGGTTTGATCCCTAGAATAGTTCAGTGGGTT
AAAGGATCTGGTGTTGCAGTGTAGATCAAGGATGTGCTTGGATCTGGTGTTGCTGTGGCTGTGGCACACACTGGCAG
CTGTAGCTCTGATTCAACCCCTAGCCTGGGAACCTCCATATGCCGAGGGTGCAGCCCTAAAATGACAAAAACAAGAA
AACAGGAATGCAAGTAAGTCAGGAGTTCCCTGGTGGTTCAGTGGGTTAAGGATCTGGCATTGTTACTGCTGTGGTGA
GGGTTTTATTCCTGGCCCAGGAACTTCTGCATGCCACAGGCACAGCCAAAATAAATAAATAAATAAATAATAAATTA
AGTGGAGTTCCCGTCGTGGCGCAGTGGTTAACGAATCCGACTAGGAGCCATGAGGTTGCGGGTTCGGTCCCTGCCCT
TGCTCAGTGAGTTAATGATCCGGTGTTGCTGTGAGCTGTGGTGTAGGTCGCAGACGCGGCTCGGATCCCACGTTGCT
GTGGCTGTGGCATAGGCCAGTGGCTACAGCTCCGATTGGACCCCTAGCCTGGGAACCTCCATATGCCGCGGGAGCGG
CCCAAGAAATAGCAAAAAGACAAAAAAATAAATAAATTAAATAAATAAATAAATTAAATAAATTAAGTAAAATTTAA
AATTTCTAGGAGTTCCCTGATGGTCTGGAAGTTAAGGATTTGGAGTTGTCGCTGCTGTGACTCAGGTTGAATCTCTG
GCCTGGGAACTTCTGCAGGCTGTGGGCACAGCCAAAAAAAAAAAAAATTAAGACAAAAAAACAAAGCAAATAATTCA
TCAGGAAGGCAGAAATTTTTTGGAAGCAGACCTAGGAGAAAATAAATATTTGTTTAAATATGTAAATGTTTATTTAT
ATTTTAACTATTTTATATATTTAACTTTCCTTTTTTTTTTTTTTTTTTTTTTGCTTTTTAGGGCCACACCTGAATTA
TATGGAAGGTCCCAGGGGAGGGGTCAAATCAGAGCTGCAGCTGCTGGCCTACACCACAGCCACAGCCACTCGAGATC
CGAGCCACGTCTGCGACCTACACCACACCACAGCTCACGGCAACGCCAGATCCTTAACCCATTGAGCAAGGCGAGGG
ATCGAACCTTCAATATCATGATTCCTAGTCAGATTTGTTAACCACTGAGCCATGACAGGAACTCCAGTCATCTTTTG
TTTTGAGGACATAAAGTAAGAGGTATAGAGAAGCACTTCCCCAGGGGTCTGAACAATGTATAGGCTATTTAGGGAAA
CAGGTGGTTATTATAACTGGAGGTTTGTACTTTTTTTTTTTGGTCTTTTTGTCTTTTCTAGGGCCAAACCCATGGCA
TATGGAAGTTCCCAGGATAGGGGTCCAATCAGAGCTGTAGCTGCCGGCCTACACCACAGCCCATAGCAACGCCAGAT
CCAAGCCGCGTGTGGAGCCTACACCACAGCTCACGGCATCACCGGATCCTTCACCCACTGAGCGAGGCCAGGGATTG
AACCCGAAACCTCATGGTTCTTAGTCAGATTCGTTAACCACTGAGCCACGATGGGAACTCCAGAAGTTTGTACCTTT
TGACCACCTTCAACGAGGGGCTATTTAGGGAAACAGGTTATGTTGTCCCAGTGCTGAGCCCTAGATCCCGAGATGCC
CAAATGTTCATCAGTAAATATATGTGTTTTTTTTTTTTTTTTTTGCCACACCAGCAGCACGCAGAAGGTTCTGGGCC
AGAGATCCAACCTGATCCACAGCACCGACAATGCCAAACCTTAACCACTAGGCCACCAGAGAACTCCTATGTATTTT
TTTCTTCCAGTTTATAATTCACCTACAGCACTGAATGAGTTGTAGAGCATAATGACTGGACTTGCATACGTCATGAA
ATGATTACCACAATAAGTTTAGTGAGTGAGTTCCCACTGTGGCTCAGCAGTAACGAACCTGACTGGTATCCATGAAG
ATGCGGGTTGGATTCCTGGCCTCGCTCAGTGCGTTTAAGGATCTGGCATTGCTATGGCTGTGGTGTAGGCGGGCAGC
TGCAGGTCTGATTCAACCCCTAGGCTGGGAACTTCCATATGCCACAGATGCAGCCTTAAAAAACACATAAAAATAAA
AATAAGTAAGTTTAGTGAACATCCATTAGCTCACATAAATAAAAAATTAAATAGAAAAAAATTTTCGTTGTGATGAG
AACTTATAGGATTTATTCTCTTAACCACTTTCTTTCTTTCTTTCTTTTTTTTTTTTTTTTGTCTTTTTGCCATTTCT
TGGGCCGCTCCCACGACACATGGAGGTTCCCAGGTTAGGGGTCCAATCAGAGCTATAGCCGCTGACCTACGCCAGAG
CCACAGCAACTCGGACGGAATCCGAGCCGAGTCTGCAACCTACACCACAGCTCATGGCAATGCCGGATCCTTAACCC
ACTGAGCAAGGCCAGGGATCGAACCCACAACCTCATGGTTCCTAGTCGGATTCGTTAACCACTGAGCCACGACAGGA
ACTCCAGACTCTTCTTTTTTTTTTTTTTTTTAAGGGCTGAACTCGAGGCATGTGGAGGTTCCCAGGCCAGGGGTCGG
ATCTGAGCTGTAGCTACCGGCCTATACCACAGCCACAGCAACACAGGATCCGAGCCACATCTGCGACGCACATCATA
GTTCACGGCAACACTGGATCCTTAACCCACTGAGCAAAGCCAGGGATTGAACCTGCGTCCTCATGGATGCTAGTCAG
ATTCAGTTCTGCTGAACAATGATGGGAACTCCCCATGCTGACTCTTAAGATAACAGAGAGAGCCTGCCTCATCATGA
TGGCCAGATTCTGTACTTGACATGGGTCTTGAATGGTCAGCAACTGATCTCAAGGCCCTGGAATTTAGTGGCTTAGC
CTTACACTGGCACCTCAGCAGAGGGTCCCAGATCAATCCCAGGCATTCTAGTAGGTGTCCTTTTTTTTTTTTTTTTT
GGTCTTTTTGCCATTTCTTGGGCCGCTGCTGTGGCATATGGAGGTTCCCAGGCTAGGGGTCCAATTGGAACTGTAGC
CGCCGGCCTACCCCACAGTCTCAGCAACGCGGGATCCGAGCCGTGTCTGCGACCTATACCACAGCTCACGGCAATGC
CGGATCCTTAACCCACTGAGCAAGGCCAGGAATCGAACCCGCAACCTCATGGTTCCTAGTCGGATTCGTTAACCACT
GAGCCACGACGGGAACTCCTCTTTTTTCTTTTTAATGGCTGCACCCACACCATATGGAAGTGCCCTGGCCAGGGGTC
AAACTGGAGCTGCAGCTGCTGGTCTACACCACAGCCACAACAACACTGGATCCAAGCTGTATCTGTGACCTACTCCA
CAGCTCGCGGCAACGCCGGATCTTTAACCAACTGAGTGAGACCAGAGATGGAACCCGAATCATCACAGAGACTGTGT
GGGGTCTTAATCCACTGGACCACAATGGGAACTCCGAGAATATGCCTTTATGGTAGGGAGTCTGACGCCTGGGAAAC
CTTTATTCTGGCAGGGCGTGGTTTACCGCAGTGATCGCCTCCCTCTAATTGCCTGCATCCCATCCCTGTGCCGGGCT
CCAGGTGAGCTGACTCCACAGAGCTCTCCTCACCTGCCGGGGCCCTTGTGACTTCTCTCTTCTCTGGTCCCCCAACC
CTGCTGCTCAATCCTACTAGCGGACTGAACCGAACGAGGCTGCCACCTCCTCAAGGCAAGGACCCTGGGTTCTTCAC
ATTATTTGAGTCCACAAGGTAGGACCAAAGGAAAATTTGTGGAGGACAGTGATGCTGGAGATGATCTGTGATATAAT
TTCCAGCAAGTAACCTTCAAGGACCCAGCAGCCATCTTTTTTTTTTTTCCACTGTACAGCAAAGGGATCAAGTTATC
CTTACATGTATACATTACAATTACATTTTTTCCCCCACCCTTTGTTCTGTTGCAACTTGAGTATCTAGACATAGTTC
TCAATGCTATTCAGCAGGATCTCCTTGTAAATCTATTCTAAGTTGTGTCTGATAAGCCCAAGCTCCCGATCCCTCCC
ACTCCCTCCCCCTACCATCAGGCAGCCACAAGTCTCTTCTCCAAGTCCATGATTTTCTTTTCTGTGGAGATGTTCAT
TTGTGCTAGATATTAGATTCCAGTTATAAGTGATATCATATGGTATTTGTCTTTGTCTTTCTGGCTCATTTCACTCA
GTATGAGAGTCTCTAGTTCCATCCATGTTGCTGCAAATGGCATTATGTCATTCTTTTTAATGGCTGAGTAGTATTCC
ATTGTGTATATATACCACATCTTCAGAATCCAGTTATCTGTTGATGGACATTTGGGTTGTTTCCATGTCCTGGCTAT
TGTGAATAGTGCTGCAATGAACATGCGGGTGCATGTGTCTCTTTTAAGTAGAGTTTTGTCCAGATAGATGCCCAAGA
GTGGGATTGTGGGGTCATATGGAAGTTCTATGTATAGATTTCTAAGGTATCTCCACACTGTTCTCCATAGTGGCTGT
ACCAGTTTACATTCCCACCAACAGTGCAGGAGGGTTCCCTTTTCTCCATAGCCCCTCCAGCACTTGTTATTTGTGGA
TTTATTAATGATGGCCATTCTGACTGATATGAGGTGGTATCTCATGGTAGTTTTGATTTGCATTTTTCTTATAATCA
GCGATGTTGAGCATTTTTTCATGTGTTTGCTGGCCATCTGTATATCTTCTTTGGAGAAATGTCTATTCAGGTCTTTT
GCCCATTTTTCCATTGATTGATTGGCTTTTTTGCTGTTGGGTTGTATAAGTTGTTTATATATTCTAGAGATTAAGCC
CTTGTCCATTGCATCATTTGAAACTATTTTCTCCCATTCTGAAAGTTGTCTTTTTGTTTTCTTTTTGGTTTCCTTTG
CTGTGCAAAAGCTTTTCAGTTTGATGAGGTCCCATGGGTTTATTTTTGCTCTAATTCCTATTGCTCTGGGAGACTGA
CCTGAGAAAATATTCATGATGTTGATGTCAGAGAGTGTTTTGCCTATGTTTTCTTCTAGGAGTTTGTCCTGTCATAT
ATTTAAGTCTTTCAGCCATTTTGAGTTTATTTTTGTACATGGTGTGAGGGCGTGTTCTAGTTTCATTGCTTTGCATG
CAGCTGTCCAGGTTTCCCAGCAACCAGCAGCCATCTTTTTGACTGAAGATACACTCTTCCCAGTGAGATGGAATCAG
ATGATGGGAGATACTATATGTACAAATGCTTCCCACATAGTAAGGCATCATAACACAGTAATTTTTGTTTATTCTTT
TTTGGTCTTTTTTTTTTTTATGGCCACACACTTAGCATCTGGAAGTTCCCAGGCTAGGGGGCGCATCAGAGCTGCAG
CTGCCAGCCTATGCCACAGCCACAGCAATGCCAGATCCTTAGCCCACTGAGCAAGGCCAGGGATCCAACTCGCATCT
TCGTGGATAGCAGTCTGGATTGCTACCTCTGAGCCATGATGGAAACTCCGCCGTAATCGTTATGAATGAAGTCTCCA
TTGCCCACCTCAGTGACTGGTCCATTTCTAATGACCCTGTACTTTTATTGGTACTTCCAGTAACGGAGTCAGACCCA
CCTGCCTACCCTGCTCCCTGGGCATTACAATGCTTATCTTATGAGGAGTTCAAATATTGGTATCCCAGCCACCGCAT
CCGCTGACTTAGATACTTGCAACCAGGCAGCTCAGCGCTTTTCCAATGCCCAGATACCTTAGGTGGCACATTGGAGA
TAGTTCTTGAAGTAGTGGAGAGCCAACTTGAATTTGATCTGGGCTTCGGTGTTGGCCCGATAACTGGTGTAGTTCCC
CTCCAGGGTGGCCAGCTCTGGGTCCATCACTGGTAAATGGGGCTGGTGACCTATGATCACATGTGGGCAGGACCCCA
CGAGCAGGCTCCCGAGCCCATCAATAAAGAACTCTGCCAAGAGAGGGAGAGAGCGCGAGAAGGAAACGTGAGCTTCA
AACCAGAGACCCGGGCCAATACTGCGACTCTGGGAGGAGGGCTGGGGTGGGGGGGGACATAGCTTCTATTCTGGGGA
GGTTCAGTCCCATGGCAAAGCCACTGAGTTGGAAGATCAGACAGATATCAGCAGAGAGACACAGATTAGCAGACCCC
AGGACTGGGAGGAATGAGAGGGGAAGAGGTGGGGTGCTGCTCACCAGCTGCAGCTAAACAGAGAAGGATGTCTGGAA
AAGGAGGAGCAGGAAATTCCCGTCATGGCGTAGTGGTTAATGAATCCGACTAGGAACCATGAGGTTGTGGGTTCGGT
CCCTGGCCTCGTTCAGTGGGTTAAGGATCTGGCGCTGCCCTGAGCTGTGGTGTAGGTCACAGAGGCAGCTCAGATCC
CGTGTTGCTGTGGCTCTGGCATAGGCCGGGAGCAAAAGCTCCAATTCGACCCCTAGCCTGGGAACCTCCACATGCCA
TGGGTGCAGCCCTAAAAAGGCAAAAAAAAAAAAAAAAAAAAAAAAAAAGGCAAAAAAAAGGAGGAGCAGCAGCAAGA
CAAGGAAAGAGGGAAGGGGCAGAGCTGCAGGGAGAGGAGGTAGAAGGGTGTCTCGGAGAAGCAGGAATAGCCTATGG
GAGACACGAAGGTGGAGGGAGGCAAGAGAGACCAAGAGCTCCCTAGTTTGGGGAGAAGGGGCTGCTTCCCTGAGCAG
CAGGGCCCCGCCCTCCCTCAGAAAGAGACTTCTGAAGCCAGCGCACAGCCCAGCTCGCTTCTTGCCCTTCCAGCCTC
CCCACCTGAGTGAGCCACTCGCTGCAGCCGGGGGTCGAAGCCAATTCTTTGGAGTCGCTCTGTGTGAGCCAGGAAGA
AGTTGACAACACCACTGGTCACCACGCAGTCGGGGAAGCCATCCACGGGCCGGAAAAATCCTGGCTGCTGGTGGAGA
CAGTCGCCATTCTTCCCCTGCTCCAGCAACAGCTTGAACTGGAATGTGTTTTCAATCACGCTGCCACCTACCTAGCC
AGCGGGAGGAGAAATCTGTTAGAGAACAGACTCCATATCCAAGGAGCCTGTGCCAGGAAGCCTTACTGGACTGAACC
TCAGTCACGACAAGAATTGCACTCCCTGGAGTTCCCGTTGTGGCTCAGTGGTTAACGAATCTGACTAGGAACCATGT
GGTTTCGGGTTCGATCCCTGGCCTCCCTCAGTGGGTGAAGGATCCGGCGTTGCTGTGAGCTGTGGTGTAGGTCGCAG
ACGTGGCTCGTGAGCTGTGGCATAGGCTGGTGGCTACAGCTCCAATTGGACCCCTAGCCTGGGAACCTCCATATGCT
GCGGGAGTGACCTAAGAAATGGCGAAAGACCAAAAAAAAAAAAGGTAATAATAATAATAAAATAAAATAAAATAAAA
AAGAAAAAGAATTGTACTCCCTGTCTTATCTACCCTTCATGTTACACTTCCGCCAAGTCCAAAGGGCAGCAAAGTTT
CTGCTGCACTTACCCTCCAGCAAGCTCACTCTTTCCAGAGGGCCACTCCCTCCCCTCCCTTCTGCTACAAGGATCCA
GGAGGATCGAGGATGGGGGATCGCGTTTGGGTGCAGGTGAGAGGCAGCCAGCGTGCAGCCGTCCCTACGTGGACTTC
CTGAGCAAGCCTTTGTCTCAAGTTGTCTCCCTCCCATTCTCTGCCCCTGGCTCACTTCTCTGCGCCGTCTGTCCACA
CACCACACACTCCTGGGAGCTCGCAGCTTTGTGTGAGCCCGAGCACAGCAGGACAAGCAAGTACATCTATTCCTGAA
CCATCATAATCACCTAGGGAGGCAGAGCAGAATCTGCCAGTTGCCCCCCACCCCCTCGCCTGTTCTTTCCTTCCTCC
TCTTAGGAAATGAGCCCCCTGAGGTGTTTTTTGGTTTTTGTTTTTCCTTTTTCAGCTGCCCCTGCAGTTCCCAGGCC
GGGGATGGAATCCAAGCCAGAGCTGCACCCACCCCACCCCCACGCAGCAACGCTGGATACTTAATTTAACCCACGGC
ACAGGACTGGGGATTGAATGGGCACCTCCACAGAGACAAACTGGATCCTTAACCCCCATGCCACAGTGAGAACTCCA
AACTCCAAACCCTCTGAGATTTAAGTGGACTAAATTAAGCGACAATGATCCTACGAAAGATGAAATTTCCCCACTTC
TCTGGAGTTCCCAATGTGGCTCAGCGGTAATGAACCTGACCAGTATCCATGTGGACGTGGGTTCACTCCCTGGCCTC
CTCGAGTGGGTTAAGGATCCGGCATTGCCGTAAGCTGTGGTGTAGGTCACAAAATCAGCTCAGGTCCCATGTTGCTA
TGGCTGTGGTATAGACGGGCAGCTGCAGCTCCAACGGGACCCCTAGGTTGGGAACTTCCATGTGCCCTACAAAGAAG
AAGGGAGGAAGGAAGGGAAAGAGGGAGGGAGGGAAAGAGGAGAGAGAGGGAGGGAGGAAGGAAGGAAGGCAGGGAGA
AATGGCCCACAGCATATGGCTTGAATCCCAGCTGCAGCTGCAGCAATGCCAAATCCTTTAACCCGCTGGACTGAACC
AGCACCTCTGCAGCAACCCGAAATGCTGCAGTCGGGTTCTTAACCCACTGTGTCACAGTGGGAACTCCCTGAAAGGA
TGTGATTTAGAACAGATGTCTCCAATTTTTAAAAAGACCACATTCTTCTCATCTTTTCCTTTTTTTTTTTTTTTTTT
TTTGGCTTCTTAAGGTTGAACCCACGGCATAGGGGGTTAGTGGTTAGTTTCCAGGCTAGGAGTCAAATTGGACCCAC
AGCTGTTGGCCTACACCACAGCCACAGCAACGCCAGATCCAAGCCTCGTCTGTGACCTATACCATAGCTCCCAGCAA
TGCCAGATCCCTGACCCACTGAACAAGGCCAGGGATCGAACCCACATCCTCATGGATACTAGTCAGATTCATTTCTG
CTGCGCCACGAAGGGAACTCCCAAGACCACATTCTTAAAAGAAAACTGTTGTCTTCTACTCCCTCTCTCCCCCTTTC
TTCTGACCGTGCAGCTGAGGGCCACAAAGATGGATGAACAACAGGGAAGGAAGCTGGACCAGGATGACCCTGGAAAG
AGACAATAGGGCCAGCTTGCATTCTCTCTTTTTTTTTTTTTTTTTTTTTTTTTGGCTTTTTGCTAATTCTTGGGCCG
CTCCAGCAGCATATGGAGGTTCCCAGGCTAGGGGTCCAATCGGAGCTGTAGCCGCCGGCCTACGCCAGAGCCACAGC
AACGCGGGATCCGAGCCGCGTCTGCAACCCACACCACAGCCCACAGCAACGCCGGATCGTTAACCCACTGAGCAAGG
GCAGGGACCGAACCCGCAACCTCCTGGTTCCTAGTCGGATTCGTTAACCACTGCGCCACGACAAGAACTCCCCAGCT
TGCATTCTTACACGGGTAGGAACTGCACCTTTTTTGTCATTTATGCTATTGTGACTGGGTCTCTAGAAGAGTAGCAA
AGAGACATCTTCGTCAATCCAGATGTTTTGGGGGACTGTCCACCTGGAATAAGAGATAACTGTGGTCACGGTGCTAC
TTATCCACTTTCTTTCCAGGCCGGGATAGAACCAGCACCACAGCAGTGACAATGCTGGATCCTTAACCCTATGAGCC
ACCAGGGAACTCCCATCTTTCTTTTTCCAAACAGCTTTATTGAGATATCTTTGATATATTAAAACTGTATGAAGGAG
TTCCTGTCGTGTCTCAATGGTTAACAAATCCAACTAGGAACCATGAGGTTGCGGATTCGATCCCTGGCCTTGCTCAG
TGGGTTCAGGATCCAGCATTTTTGTGAGCTGTGATGTAGGTTGCAGACGCGGCTCGGATCCTGCGCTGCTGTGTCTC
TGGCGTAAGCCGGTGGCTGCAGCTCCGATTGGACCCCTAGCCTGAGAACTTCCATATGCCGCGGGAGCGGCTCAAGA
AAATGGCAAAAAGACAAAAAGACAAAAAACAAAACAAAACAAAACAAAACAAAACAAAAAACTGTATGTATTGAAGG
TGTACAGCTTGATTTTTTTTTTTTTTTTTGGTCTGTGGCATGTAGTGGCTTGATGCAGGATCTCAATTCCCAGACCA
GGGACTGAACCTGGGCCACAGTGGGGAAAGCACCAAATCCTAACTACTACACCACCAGGGAACTCCCTGCAGCTTGA
TGTTTTGATATATGTAGACACTGTGAAAAGATCACCACACGCAAGCTAATTAATGAATTCATCACCTCTACACAGTG
TGGGTATCTTCACAAATTTCAAGAACGCAATGCAGTATTATTAACTATTCATCACCTTTTTTCCCCCTTTTCCATGT
GTAAATTAACTTTTGATATTTGTGGGGTTTTTTGTTCTGTTTTGTTTTGTCTTTTTAGGGCTGCACCTGCAGCATAT
GAAAGTTCCCAGGTTAGCAGTCCAATTGGAGCTGCAGCTGCCAGTCTACGCCACAGTCACTGCCACAGCCACAGAAA
TGCCAGATCTGAGCCACGTCTGGGACACACACCACAGCTTATGCAACACCAGACCCTTAACCCACTGAGCAAGGCCA
CGGATTGAGCCCACATCCTCATGGACACTAGTCGGGTTCATTACTGCTAAGCCACGACGGGAACTCCTGTGTTAATT
TTTTATTGTCATTAAGGCCACGTGTGCTTTTATAGCTTTGTGCCATTTTCATTTTTGTGATGGTGTGTGACAAAACC
AGAGCAGCACTCACATTCCTCTCCAACTCTCACCAGTCCAGAGAGGAAGTTGGAAGTGATGCATACAAAGAAAACCA
CAGCTTTCAAAAGATACACGCACCCCAACGTTCACGGCAGCACTATTCACAATAGCCAAGACGTGGAAACAACCTAA
ATGTCCATCAACAGATGAGTGGTGTACACACACACACACACACACACACACACACAATGGAATATTACTCCCTCATG
AAAAGAGTGCAATAATGCCATTTGCAGCAACGCAGATGGACCTAGAGATTATCATACTGAATGAATTCAGAGAAAGA
CGGATATCATATGATATCCCACATATGTGGATTCAAAAGAGATACAAATGAACTTATTTACCAAAGAGAAACAGACT
CATAGATTTAGAAAACAACCTTATGGCTACCAAAGGGGAAAGGTGGCTGGCGTGGGGAGGGGGTGGAGGGATAAATT
AGGAAATTGGGATTAATATATACATACTACCATATATAAAATAGATAGGAGTTCCCATTGTGGCTCAGTGAGTTATG
AACCCAACTGTGATCCATGAGGATGCAGGTTCAATCCCTGGCTTTGCTCAGTGGGTTAAGGATCCGGTGTTGCTGTG
ACCTGTGGTGTAGGTCACAGATGCAGCTCAGGTCTGATGCTGCTGTGGCTGTGGTGTAGGCCAGCAGCTACAGCTCC
GATTTGACCCCTAACCTGGGAACCTCCATATGCCTCGGATGCAGCCCCAAAGACCAAAAAAAAAAAAAAAAAAAGAT
AACTGACAAGGACCTACTGTATGGCAAAGGGAAGTACACGCAATTATTCTGTAATTTCCTACGTGAGGGAAGGAATC
TGTAAAAGAATGGGTATAGCTGAATCACTTTGCTGTACACTTGAAACTGATACACCATGGTAAATCAACTCTACTCC
AATAGAAAATACAAATTAGGGTTTTATAAATTTTATAAAAATAAAATAAAACCTAGGCCACCTGGTGGCCTAGAGGT
TAAGGATCCAACATTCTCACTGCTGTGGCACAGGCGGGATCAGGCTGGATCCCTGGCCTGGGAACTTCTGCATGACA
TAGGTGTGGCCAAGCAAAAAAAAAAAATTCAATTAAAAAAAATGACTGGGAGTTCCCATTGTGGCTCAGTGATTAAG
AAACCCAACTAGTAACCATGAGGTTGCAGGTTTGATCCCTGGCCTCACTCAGTGGGTTAAGGATCTGGCCGGCATTG
CTGTAAAGTGTGGTGTAGGCCAGCAGTTACAGTTCCAACTGGACCTCTAGCCTGGGAACCTCCAGATGGGGCAAGTG
TGGCACTAAAGACAGAAGACAAAAAAAAAAAAAAGATTGAAAAAAGTGCCTAAACACACTTTTTTCTTTTGCCATTT
CTTGAGCTGCTCCCTCAGCATATGGAGGTTCCCAGGCTAGGGGTCCAGTCGGAGCTATAGCCGCTGGCCTATGCCAG
AGCCACAACAACGGGCAATTCAGCCGCATCTGCAAACTACACCACAGCTCACAGCAATGCCGGATCCTGAACCCACT
GAGCAAGGCCAGGGATCGAACCCACAACCTCATGGTTCCTACTCGGATTCGTTAACCACTGAGCCACGACGGGAACT
CCACAACACACTTTAAGGACAGAACAACGGTGAGTCTGGGGAGTGGGGTTGGTGTGATTTGTTCAAAGAAAAGTAAG
AATGGAGGCAGAAGCAGAATCCGAGGGTCTCATTTCCGTGCGAGAGTCTCAATCCCAGAGCTGCTCTGCATCACCTC
CTGCACGGCCCTTCCCCTTCCGCCTCCCTCTTCCCCCCCCCCCCACCCCCGTCCCTTTTCCTCTCCTCTTTCCTCCT
GTCCTTTCCTCTCTGCCCTCTCCTCCCCCTCCCCCTCTGGCTCGTCAGATGGCAATGGGGTAGAACTGGCAGCGCTC
AGCTCACTTACCACGTCCAGTTCCGTTTTCTCTAGGACGTCCACCAGCGCCTCGATCCTGGTCTTGCTGTTGAAGAT
GAAGTCATCGTCCACCCAGAGCACATATTTGGTGGTGACCTGAGATATGGCCAGGTTCCTGCCAGCAAACCAGCCCT
GCGAGGGCAGGGAGGTTAGACCCGTGGTTGCCCGCCCCGCTGCCTCCTAGCATCACCTGGGGGCTTTCTCAGCTCCC
AAGGGTCAGGCTGCCCCCCAGACAGTGGCTGAGAACCTCTGGGCTAAAGGGAGTCCATGTCTCAGAGACCCTGGAAG
AAGGAGAGGGACTCTCTGGAGACGAGAAAGTCCCTCCTTGGCCCTGTGGCTTGAGGGATGGATGCAAGTCCCTTTAC
ACCTGACAGTCTTTGTGGCCCTTTCGCCCTGTGTTGCCTGGAAGATGCTGGAGGGTGGGGCTCTCTGGAAGGGGTAA
CATCCACTTCCTCCCGGTGTGCTCGAGGGAAGGTGTGGGGCGCGGAGAGAGACACCCCAGCAAGGGTGAAATCATGA
CAGAGGTTTCTCTGCTGTGGGACCTGCGTATCAGGAAACCTTAGAGCGTCAGACACCGCCAGTCGCTTACAAGGACC
TCCATCAATTTCCACACCAAGCGTGAGGAAAGACAGATTACCCACCCCGTCACTGCAGGAAAGGGAGAGTGACCTGA
TTTCTCCGGGAATTTGGAGGCAGCCAGGGGACTCAGAGGAGTCCCCACCCCCCGCCCCCCAAGGATCCTGCTGCCGT
GGGAGGGTCCCCCCCAACCCCGAAGCAGCCCCAACCAGGGTACCACTTGACCCTGGGGCCCTCTGGTCCCAAGGTGC
CCGTGTCTCCCCCTCTGGGAGGAATATACCTTCCCAAATGGCATGGTGTAATACTCCACGTGGCTGTCAGTGATTTT
CAGGGGCTCCTTGCTGTCATCGGCCACGATCACCGTCAGGTCTGGGTAGTACTCACGAACACTCCGGAGCATGGTCA
TGAGCTTGTGGGGACGGAGGAAGGTTTTGGTGGCAATGGTCACCAGGTCTCGGAGCTTCCTCTCTGGGCAAGAAAGG
GTAGGTGTCAGAGCTCTGTCTTCAAGAATCCTCACTGACGTGCATTGCTCTGGAGGTTTCTTTACACGGCGCTGTCT
CGAGTGTTTGTGGACCTCATGCCTTTTGTTCACAGTTGATGTTAGTTGGATCAGAAAATACATTTTATTATTATTAT
TTTGTCTTTTTGTCTTTTTAGGGCCGCACCTGCAGCATATGGAGGGTCCCAGGCTAGGGGTCAGCTCAGAGCTACAG
CTGCCGGCCTACACCACAGCCACACCAACACAGGATCCGAGCCTCATCTACACCACAGCTCACGGCAATGCCGGATC
CCTAACCCACTGAGCGAGGCCAGGGATCAAACCTGCATCCTCATGGATGCTAGTTAGATTCGTTTCCGCTGAGCCAT
GGTGGGAACTCCATGAGTCAGATTCTCAACCCACTGAGCCACAACGCGAACTCCCAATTTGTTTAAATGGTTTCTGT
CTTCTAGAGTGTCTCCCTTTTTTTTTGGTTTTTTTTTTGTTTTTTGCTTGTTTGTTTGTTCTTTTCTTAGTAGCTGC
ACCTGCAGCATATGTAGGTTCCCAGGCTCCCAGGCTCCCAGTTGAATCAGAGCCGCAGCTGCAGGCCTATACCTCAG
CCACATCAGATCTGAGCCGCATCTTTGACCCACATCACAGCTGGCAGCTATGCAGATACTGAACCCACTAAGTGAGG
CCAGGGGTTGAACCTGCATCCTCACAGACACCATGTCAGGTTCTTCACCCACTGAGCCACAACGGGAACTCCTCTCT
TCTGGTTCTGTTGGCTCCAGTCTGCTGTTTCCTTCTGTCGAGTGGGATGCTTCAAGTTCTGCCTGCCTATCTGCACT
TGGTTTGCAACCGGCTTTCATGCTGTTACTGGGAATTGAGACGCATAGAGTTTCACCCATCAAGGGATTCAATATGA
CCAGTCGTGAGGCCCAGGAAGAGGGGAAAAGATTTAAAGACCTGAGACCTGCCCTGTCACAGCTGCAATCCTACAGA
GAGACGTGCCTGGCCTGGTTTGTTTTTTTTTTTTTGCTTTTTTTAGGGCCGCACCCACGGCATATGGAGGTTCCCAG
GCTAGGGGTCGCATTGTAGCTACAGCTGCTGGCCACAGCCACAGCCACAGCCACAGCGATGCCAGATCCGAGCCGAG
TCTGCAGCCTATACCACAGCTCATAGCAACGCCGGATCCTCAACCCACTGAGCAAAGCCAGGAATCGAACCTGAAAC
CTCATGGACACTGGTAGGGTTCGTTAACCCCTAAGCCACGACGGGAACTCCTTGTGGTTCTTATCCATGTTCTTTTC
TTACTGATTCATAAGTCCTCTGAAGTAAAATTAGACCTTTGACTTTCGTGTGTGTGGTTATTTTTCCCCAGTTTGTC
TTTTGTCATTTGACTTTGCATATGGTAGGCTTCCGTCATTAAAAACATTAAAAATTGTTATATAATTTATGTTTTTA
GTCTTTTTCCTTTTAGTCTTTTTCCTAGGTTTTGTGTCTTATTTAGAAAAGTCATACTTTACACAGTTATTTTTAAA
CTCCAGGCTGATTCCTAGTACTTAAAACAATTAGATATTTGCTCTACCTGGACTGTACCTTGGTGTGAGCTATGAGA
TGGATTCAGCTTGTTATTTTCACACAGCTACACAGTTATCTAACACAATCTCTTGAACAATCCATCTTTTTCCCCTT
TAATTTGAAAAACTACCTTGATCACACGGTAAAATTCCAAGATGTCTATTTCTGGGTTTCTTTTCTTTTCTTTTTCT
TTTTTTTTTTTTGTCTTTTCTAGGGCTACACCCGCGGCACATGGAGGTTCCCAGGCTAGGGGTCGAATTGGAGCTGC
AGCTGCCAGCCTATGCCAGAGCCATAGCAACATGGGATCCAAGCCGCGTCTGTGACCTACACCACAGCTCATGGCAA
TGCCGGATCCTTAACCCACTGAGCAAGGCCAGGGACCGAACCCGCAACCTCATGGTTCCTAGTCGGATTAGTTCGTT
AACCACTGCGCCATGACAAGAATGCCTAGGTATCTAATTTGATTCCACTGACATAGCTCTTCGTGGTCCAATACCAT
TCTATTTTTATAATTATTACTTATTAAAATGTCATAAATCATTAGATTTTTTTCAAAATAAATTCAACCGTACAATA
AGTTAAACGTAATGAAGCAGTATTAAAAGCGTATTCTAGCATTTTTTTCCTCCAAAAAAGCTTGTTGGAGTTCTCTG
GTGGCCTAGTGGACTAAGGATCCAGTGTTGTCACTGCTGTGGCTTGGGTCACTGCTGTGGCACAGGTTCCATCCAAG
GCCTGGAAACTTCCACTCTGCGGGCACAACCAAAAAAAAAAAAAAAGCTTGTTAACAGGACTCCTATTGGAGTTTTT
ATTTCATCGAGTCTCCTCCTCCATCTCAGAGGGGAGCCCTTCTGCATCTCACCCAATAGTCTCCAGGGACCCACCAT
GGAGCCCCAGGGACAAGGGTCTTACCTGGTCCAGGGTCATATAACTTGGGCATGACAGGATAGCGGATGGTCACTGG
AAACTTGGCCACTGAGGACTTGGACTCCAGACTCACTGGAGGGAGAAATCAGGTCAGGGCTGGTGCACGGTATCTGG
GTCACTCCCCACAAGGCCGGGGAAGCCCACGCGATGGGGGAGTGAAGGACTGAGGACCCCACAGAGTCTATGGCATT
CTGGCTCCTACCCTGCTGTGTGTTCCGGAAGCAACCTGCTGACCGCCTCTGAAACGCACATGTCTGCCCCCGTGAGA
CTCTGTCGGGTGAAGTGGGCTTGGAATCAGAGGGGTAGATTAAGTTTGACTCTGCATCTATAATTTGAAATACCTTG
GGTAAGTCACATCACCTCCACCTCCACCTCCAAAACCAGGGTAACACTACCAGCCCAGTTCACCTCACAGTGCCTTT
TTTGTTTTTTTTTTTTTTGAAGGGCTGCAGGTGCAGCATATGGAGGTTCCCAGGCTAGGGGTCAAATCAGAGCTGTA
GCTGCCGGCCTACACCACAGCCACAGCCACAGCCACATGGGATCCGAGCCACGTCTACAACCTACACCAGTGCCTGG
CAACACCAGATACTTAACTCACGAGTGAGGCCAGGGATTGAACCTGCATCGTCATGGATCCCAGTCAGGCTCGTTTC
TGCTGAGCCACAATGGGAAGCCCCTTCATAGGGTCATTCTGTGGTAAGACATGTTTAAAAATCCCAAGGTACAGAGA
ACTCTCTCTCTAGCTTATGCTCATGGAAAATCTGCCTCACATTCACTGGGGTCCTGGGAAAGCCTCCTGTGTATCTG
GTCAAAGCAGAAAAAGGTAAATGTCTTTTTTTTTTTTTTTTTTTTCTTTTTACGGCTGCACCTGCTGCATATGGAAG
TTCCCGGACTAGGGCTCAAATTGGAGCTGCAGCTGCCGGCCTACGCCACAGCCACAGCCACAGCCAATGGAATCCCA
GCCACATCTGCGAATTATGCCGCAGCGAGGCCTGGGAGCAAACCTGCATCCTCATGGATTCTAGTTAGGTTCTTAAT
CCACTGAGCCACAAGAACTCCGGAAAAGGGTAATTTATTTATGTATGTATTTATTTATTTTTGTCTTTTTCTTTTTA
GGGCTGCACCCGTGGCATATGGAGGTTCCCAGGCTAGGAGTCCAGCTGGAGCTATAGCCACCAGACTACACCACAGC
CACAGCAGCTCAGAATCTGAGCCACTTCTGCAGCCTACACCACGGCTCACGCAATGCCGGACCCTTAACGCCCTGAG
CAAGGCCATGGATCAAACCCGTGTCCTCATGGATACTAGTTGGGTTCGTTAACCACTGAGCCACAATGGGAACTCCC
GGAAAAGGGTTTTAATTCATCCAGAAAGTAAGTGGGGCTGCCCTGAGGGTGGCAGGAATTGGTCTCCCATGAATTCT
GGGAGTAAGAGTCGGGTTTGGGATGGGAGGGGAGGAGGAAGACAAAGCCACTGCCCTTGGGACTGACAGCTCCCCCA
CATCCCTCTTTCCCGTAATGCTCAGGACAAGCCACTGACACGTGGACTGTGTTCTCCTCTACTGCAGCTGAAACCTT
CAGCTTTTTCTTTTTCTTTTCTTTCCTTTGCTTTTTAGGGCCGCACCCGCAGCATATGGAAGTTCCCAGGCTAGGAA
TCGAATAGGAGCCGCAGCTGCCAGCCTACACCACAGCCACAGCAACGCAGGATGGGATCTGAGCCACGTCTGCGACC
TACACCACAGCTCACGGCAACGCCGGATCCCCGACCCACCGGTGAGGCCAGGGATCGAACCGCCAACCTCGTGAATA
CTGGTCAGATTCATTTCCACTGCACCACAACCGGAACAGGGAACCTTCAGCTTTGATCACTGATGAGAACGGGAGCA
GAAGGGGATGGTTTCCAGGTGCAGAGCATGAATGATCTGTCCTCATGTACAGACAAGCAGGCATTTCACTGTCTTTC
TTTCGGGTCCCTCCACGGGCTCAATGGCAACACGGGGATAGTACCAGGTACACTAAGTGGGAAATTAGAAACAGGAG
CCAGGGAAGCAGGCTTCCTGGAGAAGGAAGACCTTGAGAGCCGGGGGCGGGGGCAGTGGTGGTGTTTATGGGGTCCC
TCAGCATTTTGCCATCCGAGGACGGACTCACCCACATCCACTCTGTGGAGGTGGTACTCTGTGCTCGTGTATGTCAC
ATGCTGGAGGATGAAATTCAAAAGCTCCCGGCTACTGGTCAAAATGTTCAGCTGCTTCTGGCCTCTGCCCTTCACCA
CATTGTCTGGGACGTCAGCAAGGGTGTTCAGTGTCCCCAGAGAAGCTGTCAGGGTGACCTAGGATAAAGGAGGTAGA
AAGCCTAAATGCAGAGAGGCACATACCCAGGATGGCCAGCAGGGGGCAGCATGCATAAGGGTGTGAGGAGAAGAACG
CTTCATGCTCCCGAAAGCTAGGGTCTGGCCTCTGATGGAGTGTCTGCCCCAGCCCCAAAAGCCTAGGACCTAGGACC
TGGTGTGTTCAAGGGCCATTTCTGAAACATTCTTAACTCTTGGCATGCAGAGTTAAGTGGCATCCATTCTTAAAGAT
TTCTTCTGGAGTTCCTGTTGTGGCTCAGTGATAACGAATCCGACTAGGAACCATGAGGTTGCAGGTTCGATCCCTGG
CCTTGCTCAGTGGATTAAGGACCCAGTGTTGCTTCGAGCTGTGGTGTAGGTTGTAGATGCGGCTTGGATCCGGTGTG
GCTGTGGCTCTGGCGTAGGCTGGCAGCTACAGCTCTGATTGGACCCCTAGCCTGGGAAACTCCATGTGCCGCTGGAT
GCGGCCCTAAAAAGACAAAAGACAAAAAAAAAGAAAGAAAGAAAGAAAGAAAAAGAAAACTGCTGAAAACATTTCAG
TCAACAGATCTTTTCTTTTCTTTTCTTTCTTTTTAGGGCCAGACCTGAAGCACATGGAAGTTCCCAGGCTAGGGGTC
CAATCAGAGCTACAGCATCTTTGTCTGCCCCATCTTTGTCTCTCTGTCAAACGCTGAGACCAGCCACCATCTCAGGG
AAAAGCGCATGGGCAGTGAGCCAAGGACAGGATGCTAAGTGCAAAGTGGGGCTGGGAAGGGGACTCTTGCCTCATAG
ATGGGAGCATCAGGTCCTTCAAACCGGAGGCCTGGAGGTCACAGGAAAAAGGAGAAAGGAAAAAAAAAAAAAAAAAC
ATTTGAGAGGATGCCAAGAGTTCCCTGATGCTCTCAGCTCCCTGGCCAATTCCTACACATCCCTCCAGAGCCCCTTC
AAGTGTCACCTATCCAGGGTGTTTGCAGACCGCTCGCCTCCCCACTAGAGCTTGCTAGATGGTGTCCAACGGACCTC
TGCAAACTCCAGCAAACCAAAGCCTCTGATGCCCTCCCCTAGTTTGGGTTTTTTTTTTTTTTTTTTTTGTCTTGTTG
TTGTTGGGTTTTGGGGGGGGGTTGGGGGCTTTTTAGGGCCACACCCTCTGCATAAGGAAGTTCCCAGGCCACGGGTT
GAATCAGAGCTGCAGCTGCTGGCCTACGTCACAATGACAGCAATACAGATTGTCAGCTGAGTCTGCGACCTACACCA
CAGCTCACAGCAACACCGGATCCCTGCCCCACTGAGCGAGGCCAGGGATACAACCCAAAACCTCATGGTGCCTAGTT
GGATTTGTTTCCACTGCACCACCACAGGAACCCCTAAATGGTAAACTTTATGTTACATATATTTTACACACTAGAAA
GAGAATTATCCAAAATGGCAAATCATTTTTTAAATGAGTACTTAAAAACACGAGCAACTCAGAGTTCCTGTCATGGC
GCAGTGGAAACGAATCCAACTAAGAACCATGAGGTTGTGGGTTCGATCCCTGGCCTCACTCAATGGGTAAAGGATCC
AGCATTGCTGTGCGCTGTGGTGTAGGTCGCAGACGCAGCTCGGATCTGGTGTTGCTGGGGCTCTGCTGTAGGCCAGC
AGCTACAACTCCGATTTGACCCCTAGCCTGGGAACCTCCAGGTGCTAAAAAGACAAACGACAAAAACAAAAAACAAA
AAACAGAACAAAACAAAAAAAACCCAAAACACCAGCAACTCATCTCAAATGTTTTTACTTTAAAATCTATCTCTGTT
CTTATGACTAATGCAAATTCTCACTCAAACACATCCTCCTTCTGTGGCCTAAACTTATTTGGGAAATTGGCAAAATA
ACATTTACCTCACAGGGATGTATGCTGGACGAGAGGTGTGTGTAAAAACCACTCGTGGAGGAGCTGTAACGGATAGA
AATATTCTTTCCATATGCAGTCCCTGGAGATGGGCTGAGGCTTTGCTTGCTCCCTTGATGCTGGCAGACACCAAAAA
GCCAATAATGGCCTAAGATTCCTCGAGGCACCCAGATCTCCGTCCTCTCCTATACGATCCAAGATGCCCAGGGAGGC
AACAGCTCCTAAGTGCCATTCCCAGTGGTGGAAACAGTGAGAATAACATCAAATGAAACCATGTCCAGCTTCATGGA
TTGTGCTGGGTATCCGGGAAGGATTCAGCGGATAACTGCTCCCTTCTGCTCCCTTCTTTGCTTCAGAAGGACTACGA
GAGCTGCCTGGGTCCTGTCCGGGTGGAGATGCACCTACCTGGGATGGGGATGGTGTGTAGAGGCATCACTTCCACCC
CGTGGACCGGGTACCCAAAGGGGAGGTTGGGCTGAGCCAGCAGGGGCGGTGGGCGAGGGAGCCCTTCTCTGCAGGGA
AACAAAACCATCAGCAGCTGCCTTGATACCTGTCCCTGACTAGCTCTTTTTTGGGGGGGAGGGGGGTGCAACCACAC
CCACGGCATAGACGTTCCCAGGCCAGGGATCTCACCCACCCCACGGCAGCGACCTGAGCCAATGCAGTGACCATGCC
AGATCCTCCTTAACGTGCTGAGCCACAAGGGAACTTCCACTGCTCCCACTGGTTTGTTCTTTTTTTTTTCTTTCGTT
TTTGGCCTTCCCAGGCCAGGGATCAGACCTGAGCTGTGGCTGCGACCTAAGCTGCAGCTGCAGCAAAAGATCTTTAA
CCCACTGTGCTAGGCCAGGGGTTGAACCTGCATCCCCGTGCTCCCCAGACACAGCTGATTCCACTGTACCACAGCAG
GAGCTCCTCACTGTCGCCACTGGCTAGTTCTTTTTCTTTTTTTCTTTCTTTTTTTTTGCTTTTTTAGAGCCACTTCC
CGCGGCATATGGAGGTTCCCAGGCTAGGGGTCCAATCAGAGCTGTAGCTGCCGGCCTACGCCACAGCCACAGCAACG
CGGGATTTGAGCCGCGTCTGCGACCCACACCACGGCTCACAGCAATGCTGGATCCTGAACCCACTGAGCAAGGCCAG
GGATCGAACCCACATCCTCATGGATACTAGTCAGGTTTGTTAACCACTGAGCCACGACAGGAACTGCTGGCTAGCTC
TTAAAGGGGTATCTGTGCCCAGAGCTTTGGGCTGCAAAGGGGGAGAAATCCAAAGTAAATCGTCGGATTGTCATGCA
TTCTCTCCTCTTCTTTATTCCTGCTCCTCCCTCCAGCCTCGAATTCCACAAAGAAACTGAGGCAGATTACAACAACA
CACATTAAAAATAAAAATCACGGAGTTCCTTTTGTGGCTCAGCCGGTTAAGAATCCAATGCAGCATTCTTGAAGTTG
CGGGTTCAATCCCTGGCCTCGCTCAGAGGGTTAAGGATCCAGCGTTGCCCTGAGCTGTGGTGTAGGTCGCAGACGCG
GCTCGGATCCCACATGGCTGTGGCTGTGGCTGTGGGGTAGGCTGGCTTCTGTAGCTCCGATTGGACCCCTAGCCTGG
GAACCTCCATGTGCCTCGGGTGTGGCCCTAAAAAGTAAATAAATAAATAAAATGAAACATAACATAAAGAGAACAAA
GGTAACACCTGCTCACACTCACCACGTTCGAATTATTTTAATACATTTTCAATTGCTGGTTTTCAATGTGAGCCATT
TTAAATAAATCTTTACATGCAATATTAAAAAATATTAAAATATTATCTCTACTCTTGAGGTTATTTGCATCAATCTC
CCTGTGGATGGAGATATTATATAACCGGCATGCAATGATATCTCGTGGGAGACTTGAAATCAGCCACAGTGTGATTT
CTTGTAGGGTTGAGTTTTTTTTTAATTTTTGAACTTTTTACTAAAGCAGGGTTGATTTACAATGTTGTGTACAGTGT
GATTATTAAACCGTGGAAATTGGCAAACACTACAAGCCACTACCAAAAGCCCATGGTTAAATATTACCACCACTATT
CATATTTCTCCCTCAACGTATAAACACATCTACCCACACTTATACACACAACTATCCCCTCCTCTTTTAAAAACACA
AATGTGGAGTTCCCATTGTGGCAGAGTGGAAATGAATCTGACTAGGATCCATGAGGATGCAGATTCGATCCCTGGCC
TCACTCAGTGGGGTAAGGATCCAGCGTTACCGTGAGCTGTGGCGTAGGTCGCAGACGCGGCTCAGATCTGGCATTGC
TGTGGCTCTGGCGTAGGCAAGAGTCTACAGCTCCAATCAGACTCCTAGCCTGGGAACCTCCATGTGCCATGGGAAGT
GGCCCTAGAAAAGGCAAAATACCAAAAAAAAAAAAAAAAAAAAAAAAAAAAGAGGGCATTCCCTCCCCCCTCCTTGG
AGCCACACCCTCGGGAATGAGTAGAGAGCTTCCGCTCCATCTCAGGGCGCAAGAGCCCTCAGCATCTGCAATACCTC
CTCTGAAAGTGTTCGAGCTCAGCCTGTCTCCTCAGGTTCACTGCGGGGAGGTCTTGCGGGTCGTAGGCATCCTCCAA
GTTATAGCTTTCCTGATGCCCGAAGGCGTCACATTGGCACTGGTTTTTCGGGAACAGCCTAAAATAAGACAAGGTCA
AAGATCACAGATTGGGAAAGTGGGCTGGTAGGTGAGGGGGAGCCGCAAGCTCGGTCCGGTGTATTTTTTTTTTTTTT
TTAACTTTTTATTTTCTCTTTTTTTGTCTTTTTAGGGCCGCAAGGTTCCGAGGCTGGGGTCTCATCGGAGCCGTAGC
CACCGGCCTACGCCAGAGCCACAGCAACGCAGGATCCGAGCCGCATCTGCGACCTACACCACAGCTCATAGCAATGC
TTGATCCTTAACCCACTGGGCAAGGTCAGGGATCGAACCCTCAACCTCATGGTTCCTATTCGGATTCATCTCCGCGG
AGCCATGATGGGAACTCCCAATCCAGTGTGTTTTTCCCCCTAGGCTTTCCCATACCTAGCGCCAGGGTTGGGTTGAG
ACCCTGGAATCACAGCAGCGGCCGCTCCCAAAGACACAGGGAAGGAAGGGAAGAGAGGAAGGAAGGAGGGCGAGAAG
GCCCCCTCTCTGGAATCAAAGTCCTTTATTTATTATTATTATTATTATTTGCTTTGTAGGGCTGCACCCGCAGCATA
TGCAGGTTCCCAGGCTAGGGGTCCAATCGGAGCTACAGCTGCCAACCTACACCACAGCCACAGCAAGATCAGATCCA
AGCGGCGTCTGGGACCTACACCACAGTTCACGGCAACCCCGATCCTTAACCCATGGAGCGAGGCCAGGGATCAAACC
CACAACCTCATGCTTCCTAGCCAGATTCGTTTCTGCAGCGACATGACAGGAACTCCCCAAACTCCTTTAAACTTGAG
AGTCACAGGAATCTCAGAGGCATTGCAGCCCCACCCACCAGATGAAAAGGCCAGAGGGCCAGAAAGGCCACATCTTT
CCTATAATTTTGTTTAGTTTTGGGGGTTTTAATGTGTTTTTGTTTTTTAGGGCCACATCTGCAGCATATGGAAGTTC
TCAGGCTAGCGGTGGAATCGGAGCTACAGCTGCCGGCCTACACCACAGCCACAGAAACATGGGATCTGAGCTGCGTC
TTCAATCTACACCACAGCTCACCGCAACCCTGGATCCCCGACTCACTGAGCGAAGCCAAGGATCAAATCTGCGCATC
CTCATGGATCCTAGTTGGGTTTGTCACCACTGAGCCACAACGGGAACTCCTCCTACAGTTTTGGTTAAATAGGCCCT
CCAAAGTCCTAAAGAACTTTGCTGGGTGCTATAGAGGCTATGCCCAGCAGACCAAGCCCCTTTCTAGTCCCGCCGTT
TGCAGTCAAATGCTCTACCCCTGAGCCATACTCCCACCAGGTCCCGCAGTCAGGATTCACATTCCCAATCAGCACAG
GTGCAGAAAGGTAGGGAACTGGCTGTAAAGTGGGCATAAGAGGACACAGTAGGAGTTCCCGTCGTGGCGCAGTGGTT
AACCAATCCGACTAGGAACCATGAGGTTGAGGGTTCGATCCCTGGCCTTGCTCAGTGGGTTAAGGAGCCAGTGTTGC
TGCGAGCTGTGGTGTAGGTTGCAGATGTGGCTCGGATCCTGCGTTGCTGTGGCTCTGGCGTAGGCCGGTGGCTACAG
CTCCGATGGGACCCCTAGCCTGGGAACCTCCATATGCTGCGAGAAGGGCCCAAGAAATAGCAAAAAGACAAAAAAAA
AAAAAAAAAAAGAAAAAAGGGCACAGTAAAGCCACAGGAGGAGCCAGGGAAGTGTCAGTGCAAAGTGGTATTCTTGC
CATCTCACCCGTTTTCACCGTAGAAATCGGGTTTCTCAGGTAGAAGCTTCAGCGTCTGCGCATCCAGGGTGGGGGAC
GGGATGGGTGAGTTGAGGAGACTGAAGTCTGTATCGAGGAACACGCTTTGGAACATAAAGAGTCCAACGCTCAGGAC
CAAAAGCACCATCAATATCTTGAGGATCGACAGACATCTAGGGCTGTTGGGACACAAGAGAGCAAACGCTGTTAAAA
TCTTTTCTGAGTATGTTAAAAAAGATTTCATTGTGCGACATAGATGGGAATAGCAACTTGAGCAAAAATGCAAGTCA
AACCTGTTTTGTACACTACGTATCAAAATTGATTTCTTCCCAAGGCAAAAGAGAAAGAAAAGCAAAAATAAACCTAA
GCAAACTGACAAGCTTTTGCACAGCAAAGGAAACCATAAAATAACCCAAAAAGATCCTGCTGGGATCCACTGGGAAC
GATGTCTGGTCACTTGCGATGGAGCATGATCATGTGAGAAAAAAGAATGTATACATGTGTGTGTGACTGGGTCACCT
TGCTGTGCAGTAGAAAATTGACAGAACACTGCAAACCAGCTATAATGGGAATGATAAAAATCATTTAAAAAACTGAT
TTCAGATAAATAGAAAAGTAAAGAATCAAATCTGCAGAGAGTTCCCTGGTGGCTCATTGGGTTAAGGATCTGGTGTG
GTCACTGCTGTGGCTCTGGTCACCACCGCGGCATGACCTCCATCCCTAGCCCAGGAACTTCTGCATACGTGGGCATG
GCCAAAAAACTATACTCAGTGGAAAATGTGAAGTTTTTCAAATACGCACTTCTGATCACAAGACCTAAAATTAATAA
ATGAAGCAATAAAATAAGAGATTTGAAAATGGACAACAAAATGAACCTACGAAAAGCAGAAACAAGATTTTAGAGAT
AGCCAAATAGAAAGTGGTGAATTTAAAAAAAAAAAAACTAAAATGGAATCATCGTTAAATCTAAGCACAGAGTAGAC
AACTGGTTTTTTCTTTTATTTTTTTAAAATTTTATGGCCACAGCCATGGCCTGTGGAAGTTCCCAGGCCAAGGACTG
AATCCAATCCATAGCTTCAACCTACACCTTTAACCACCGCACTGGGCCCAGGGATCAAACCTGCACCTCTCCAGTGA
CCTGAGCCACTGCAGTCGGATTCTTAACCCACTGTGCCAGGGTGGGAATTCCAGACAACTTTATAACCTCCTTGCTC
TAAGACTTTCCTCCTGACCCAGAAGTGACACCTACAAACGAGTCTGGTTATATCACATGACGCTCCCCTGGTCCTGG
CTGAGTAAGCGGATGTTCACCTCATCCGAATGGGGCTAATCAGCCAGAATTTCCTTCCCAGAAATGGGGAACCAGAG
ATATTGTTCGGCTAATCCTAATCCCCTGAACTGAGAATAGAGGGGAGGAAAGAAGAGAGAGAAGACAGAAGGTGAGA
GAAACAAAAGAAGCCTAGAAGGACTTCCCATTGTGGCTCAGTGGGTTAAGACCATGACCAGTGTCCCTAAGGATGCA
GGTTCAATCCCCACCCTTGCTCTGGCATTGCCACAAACTGGTGGCAGATGCGGCTTGGATCTGGCGTTGCTGTGCCT
GGGGCATAGGCTGGCATCTGTGGATCCAATTCGACCCCTAGCCTGGGAACTTCCATGTGACACAGGTGCGGCCCTAA
AAAAAAATCGTTTTTAATTTAAAATTTTGGGGGCAGTGTCTTTAAGGCATTAGTCTGCTATGGCTCCCTTTGCCTGA
CAAAGCAATAAAGCTATCTTTTTCTCCTTCACCTGCTCCTCCCCCCAAAAAAGAGTTCCCATTGTGCCGCAGCAGAA
ACGAATACAACTAGTAACCATGAGGTTTCACGTTCGATCCCTGGCCTTGCTGGGTGGGTTATGGATCCAGCATTGCC
ATGAGCTGTGGTGTAGGTTGCAGATGTGGCTCGGATCCTGCATTGCTGTGGCTGTGGTGTAGGCCTAGCCTTGGAAC
CTCCGTATACCATGGGTATGGCACTAAAAGCCAAAAAAAAAAAAAAAAAAAAAAAAAAAAAATTTAATTTAATTTTT
AAAATTAAAAAATTTTTAATTTAGTTTTTTTAACTTAAAAAAATTTTTTTAAATAGAGAAGCCTAGATCCTGAATAC
CTAGATGAAAGGGATGACTTTCTACAAAAACGCAAATGAATAATGTATTGGGGAAATAAAATAAACAAATAAACAAA
TAAATAAAAGAATTCCCACTGAAGCACCGCCCCCCCAAAAAAAAACCCACAAAAGACTTAAACAGACCTGTAAAAAT
TTAAAAAAAAAAATCAAGGAGTTCCTTTCATGCCTCAGGGGTTAATGAATTCAACTATGAACCATGAGGTTTCGGGT
TCAATCCCTGGCCTTGCTCAGTGGGTTAGGGATCCAGCGTTGCCGTGAGCTGTGGCTCTGGCGTAGGCTGACAGCTG
TAGCTCCAATTAGACCCCTAGCCTGGGAACATCCATATGCCACTGGTTCGACCCTACAAAAGCCCAAAAAAAAAAAA
AAAAAAAAAAAATCCAGGAATTTATCAAAGGTCTATGTACTTTTCAAAGTCCCAAATCCACACTTCACAAGTAACTC
CAGACTGGTTTGTAAGAAACCAGCTTTGCAGTGATGCAAATATAGGTACTGACCAATAACGATGTAAATACGCCAAA
CAAATATTAACCAGTGGGACACAACAGTATCTTAAATGAATGAGTCACCGTTAACGAATGCTGTTCTTGGAGTTCCC
GTCATGGCTCAGCAGATACGAATCTGACTAGTATCCATGAGGACACAGGCTCCATCCCTGGCCTTGCTCAGTGGGTC
AGGGCTCTGGAATTGCTGTGGCTGTGGTGTAGGTCACAGACGTGGCTCAGATCCCGCATTGCTGTGGCTGTGGTGTA
GGCCGGCAGCTGTAGCTCCGATTCCACCCCTAGCCTGGGAACCTCCATGTGCCGCAGGTGCGGCCCTAAAAAGACAA
AAACAAAAGCATGTTCCTTCTAGGAGAGCAAGGATAACTCAGTGCCACTGTGGGGCAAAACCACACCGACGCCATGC
TGTCAGCTCATCTTAGGCCCACAGTCTCATCTGCTCCCCCTCCTTATTAAAAAAAAAAAAAAAAAAAAAGAATGATC
ACATCCTAAGTTCCTAACACAATTTTCAGACTATCAGATAGAAACAAATCACTGACAACCTGGGTGGGGGGCAGCAT
TTGGGGGAAGTGAGTGTGGTCTTGGCCTTTTTGAGGGTTGGGTTTGTTTCCTTTTGCTATTAGGTACTAAAACTTAA
AATTGCATCACTTAGTGAAAACAGAACAAAAATAGGGTCGGACTTTCTCTGTGGCTCAACAGGTTAAAGACCCAGTG
TTGTCACTGCAGTGGCCCTGGTCGTTGCTGTGCCATGGGTTCCATTCCTGGCCTGAGAACTTCTGTATGCCTCGGGC
GTGGCCAAAAAAAACCCAAACAAAAACAAAAACAGAAACATGAGTTCCTGTCGTGGCGCAGTGGTTAACGAATCCAA
CTAGGAACCATGAGGTTGTAGGTTCGATCCCTAGCCTCGCTCAGTGAGTTAAGGGTCTAGCGTTGCCATGAGCTGTG
GTGTAGGTCACAGACACAGCTCAGATCTGGCCTTGCTGTGGCTCTGCCGTAGGCCAGTGGCCACAGCTCTGTTTCAA
CACCTAACCTGGGAACCTCCATGTGCGGTGCATTCAGCCTTAAAGAGAAAAGAAAAAAACAAACAAACAAACAAAAA
AAAACAATAGTGAGGAAAAGTGGCATCATTTTACCTTTTTGCCTATTTAATGTTTAGCTTAATAGATAAAATGAACC
ATCTGTTAGGACAGGTTGTTTCGCTGAAGAATATGAAGAAAATACAACCCCACACAGGTATGTCACCAGAAAAGGGA
GAAACACTTTAATTGCTTTTTCAATATTGTAGATATTTATCTTTGATACTACACCAAAAATCAAGAAGTTAGTAGCA
GGTTATTGTTTTGTTTTGTTTTGCCTGTGGCATGCATTAGCTCGATGTGGGATTTTTTTTTTTTTTTTTTGGCTTTT
TTTTTGGCCTTTTGCCATTTCTAGGGCTGCTCCCAGGGCATATGGAGGTTCCTAGGCTAGGGGTCCAATTGGAGCTG
TAGCCACCAGCCTATGCCAGAGCCACAGGAAACGGGGGGAGTTGAGCCAGGTCTGCTCACCTTACGCCACAGCTCAC
AGTAATGCTGGATCCTTAATCCATCTGACCCAGGCCAGGGATCGAACCCTCAACCTCATGGCTCCTAGTCAAATTCA
TTAACCTCTGAGCCACGACGGGAACTCCTCAATGTGGGATTTCAGTTCCCAGTCCAGAGACTGAACCTAGGCCACAG
AGGAAAAAAGCGTGAACCTGAACCCTTAGTAGCTAGGGAACTTCCAAGAAGTGGTACTTTCTTAAAAAGTTAGTTAA
GTGTGGACTCTGAAACCATATCAGTGAAAAAAAAATTTTTTTGCTTTTTTTTTTTAGGACCCCACCTGGTGCATATG
GAAGTTCCCAGGCTAGGGGTGGAATGAGAGCTACAGCTGCTGGCCTACACCACAGCCATAGCAACGCCGGATCCTAA
ACCCACCAAGCAAGGGAACAAATAGAGGGAGTTTCCACTGCGCACAATGGGATCGGTGGCATCACTGCAGCGCCAGG
GACACAGGTTTGATCCCTGACAGCATAGGTTGCAACTGTGGCTCAGATCTGATCCCTGGCCCAGGAACTCCATATGC
CACTGGCACGGCCCCTCCACCCTGCCAAAAAGAGTTTGGAGGCGTTCCCTGGTGGTTCAGTGGTTATGGATCTACAC
TCTCACCACTGTGGCCCAGGTTCAATCCCTGGTCTGGGAACTGAGATCCCACATCAAGCCGCTGCACACCTTGCCCA
AAAAACAGGGTTTTTTAACCTTTTTTTTTTTAAACTGTTATTCCCCAATGCGATTTTTTTCCCCTACTGTACAGTAT
GGTGACCCAGTTACACATACATGTACACATTCTGTTTTCTCACATTATCATGCTCCATCATAAGTGACTAGACAGAG
TTTCTTTCCTTTTTTCTTTTTTTCTTTATTTTTTAATTACTTCCCCAATACAATTTGTTAAAAGGGTTTTTTAATCC
TGATAATAAACACATAAAATTTAGTACCTTGGAGTTCCCGTTGAGGCTCAGCAGAAACAAACCTGACTGGTATCCAT
GAGGATGCAGGTTCAATCCCTGGCCTCACTCAGTGGGTTAACGATCCCGCATTTGCCATGAGCTGCGGTGTAGGTCG
CAGATGCAGCTCAAATCTGGCATTGCTGTGGCTGTGGTGTAGGCTGGCAGCTATAGCTCCGATTTGACCCCTAGCCT
GGGAACCTCCATATGCCATAGGTGTGGCCCTCAATAAAACAAAGAAAGAAAGAAAGAAAGAAAGAAGGAAGGAAGGA
AGGAAGGAAAGGAAGGAAGAAGGGAAGGAAAGGAAGGAAAGGAAGAAAGAAAAAATTTATCACCTTAACTACTTCTA
AGTGTACATATACTTTCATAATGTAGATTGTTCATGTCGTTTTAGAACGGATCTCCAGAACTTTTTTCTGCTTTTTT
CTTTGCTTATATTTTTGCATGCAACTATTTTTATCCATTTTTTCTGATTATGAAATTTTTATCTTTTACCCATTGAA
GAAAAAAAAAGTTCCTCTTTACAAAAACAAAACAAAACAAAACAAATATATGTAGGAGAAATGATAGAATTAGAAAA
ATCACCACTTTGCTACCAACAATGTAATAAATGATTCTGGCCAGGATTGTCCATCTTTTTTTTTTTTTTTTCCTCGT
TTTTTTGCAATTTCTTGGGCCACTCCTGCGGCATATGGAGGTTCCAAGGCCAGGGGTCCAATCCGAGCTGTAGCCGC
CAGCCTATGCCAGAGCCACAGCAACGAGGGATCCAAGCCGCGTCTGCAACCTACACCACAGCTCATGGCAACGCCGG
ATCGTTAACCCACTGAGCAAGGCCAGGGATCGAACCTACAACCTCATGGTTCCTAGTTGGATTCGTTAACCACTGAG
CCACAATGGGAACTCCAGGATTGTCCATCTGTTCTAAAACATTTGCCAGGTGCAGGATTTTGTTTTGTTTTGTTCTG
CTTTTTGTGTTTTTCTTCTTCTTTTTCTTTTTTCTTTTTCTTTTTTTTTTTTTCTTTTTTGTCTTTTTAGTGCTGCA
CCCACAGCATATGGAAGTTCCCAGGCTAGGGGTCTAACCACAGCTGCAGCTGCCAGCCTACGCCACAACAGCAACAG
CAACGTTGGATCCAAGCTGTGCCTCCAACCTACACCCCAGCTCACGGCAATGCCAGATCCTTAACCCGCTGAGCGAG
GCCAGGGATCAAGCCTGCATCATCATGGATACTAGTCGGGTTCATTAGCCACTGAGCCACGACAGGAACTCCTGGAG
GCAGGATATTGAATGGTGCCATTCCGGAGAACACTTACTACTTACAAAGAGATAAAAACACATCTTTGCAATGAAAG
GATCATGCATCACTACCTTAACCACATGGTCAAATAAACATCCCTAATAGTGAGGCAGCCTGACCAACTGTCCTCCG
GATATGATGATAGGAAGCACACAGATCATTTAAAGGAGTATTACTGCCAAAATATTTAACCGAAATGTAATCAAGGA
TCAGAGACCTCACTGCCAATTTATAGGAAAAAACAGGGGATAAAAATTTAGTAACACCATCAAGAACAATAGACAAA
TCAGGGACATCAGAATGTTTTCTGCAAGACAACAGGCCTGAACTCTTGACAAAGGAAAAAAAGTGGGAGTTCCCGCT
ATGGCACAGTGGGTTAGGAATCGGACTACAGCAGCTCGGGGCATTGTGGAGGTGCGGGTTTGATCCCTGGCCCGCTA
TAGTGGGTTAAAGGATCTGGCGCTGTCAAAGCTGCGGCCATTAAAAAAAAAAAAAAAAAGAAAAGAAAAAAGAAAAA
GCAATTGAAAAAAATAAAAAGAATGAGAGTGAATGAGTAACATTTCTAGTAAAGGGTTGCCTGTATCTTGTGCAGAA
CATACAGAATACATCTTTCAATGATTTTAGTCAATTTTTTTGCATTTTAAGAAATTTCTTTTTTTTTAATTGTGGTA
TAGTTAATTTACAATGTTGTGTGAATTTCAAGTACACAGCAATGTGATTCAATTACATATATACATATATACACATA
CATATCCTTTGCAGATTCTTTTCTATTATAGGTTGTTACAACATTTTTTTTTTCTTTTTAAGGCTGCATGTGTGGCA
TATGGAAGTTTCCAGACTAGGGGTCGAACTGGAGCTATAGCTGCCCGCCTACACCACGGCCACTGCCACAGCAACAC
GGTTTCCGAGCCATGTCTGCAACCTACACCACAGCTCACAGCACGCTGGATCCTTGACCCACTGGGCGAGGCCAGGG
ATCCAACCTACACCCTCATGGATACTAGTCAGATTCCTTTCTGCTGCACCACACAGGAACTCCCTATTATAAGATAT
TGAGAATAGCTGTCCTGTGGCACAGTGGGTAAAGGATCTGGTGTTGTCACTGTAGTGGCTCAGGTTGCTGCTGTTGC
ACAAGTATGATCCCTGGCCCAGGAACGCTTGGGATGGCATTAATAGGAATTGTTTGGTAGGAGATTTTTAATAAAAT
GTTCAACCGCCCAATTTTTAATAGATAACTACAAATGTTCTCCACTGTTAAAACTGCACTTTATGTACTTAAGTGGG
GATGTTAAAATTATATGGGTCCGCCCGCTATTATAGTTGAACCACATTTGAGACACATTCAAAAAAGGGTAAAAATC
GGGAGTTCCCACTGCAGCTGCGGGTTCAATCCCTGGCCTCACTCAGTGGGTTAAGGTTCCGGCATTGCCATGAGCGG
TGGTGTAGGTCGCAGTCGCGGCTCAAATCTCGTGTTGCTGTGGCTGTGGCATAGGCTGGCAGCTACAGCTCTGATTG
GACCCCTAGCCTGGGAACCTCCATATGCCGCAGGTGTGGCCCTAGAAAAGACACACACACAAAAAAAAGGTTATGTT
GAAGTTCCCGTTGTGGCTCAGCAGTAACAAACCGGACTAGTATCCGTGAGGACACGGGTTTGATCCCTGGCCTTGCT
CAGTGGGTTAAGGACCCAGTGTTGCCACAAGCTGTGGTTGCAGTGCAGGTCACAGACAAAGCTTAGATCTGACATTG
CTGTGGCTGTGACACAGGCCAGCAGCTACAGCTCAAATTCGACCCCTAGCCTAGGAACATCCACCCACAGGGGGCGG
CCCTAAAAAAAAAAATATATATATATATATGTGTGTGTATATATATATATATATATATTTTATATATAAAACATTTT
ATATATATATATAAAATATATATATATAAAAATATATATATATATAACATTTTATATATATATATAAAATGTTAACA
TTGAGTAGGTTTAAGGTTATTATTTTAATAACTTTATAAATAAAAATTTTAGATTTTCTCAGCTTTAATTTTTAATT
AGGTGTGGAGTTCCCACTGTGGAGCAACAGGATCAGCAGCATCTCTGAAGCGCAGGGATGCAGGTTTGATCTCCAGT
CCTGCACAGTGGGTCAAAGATCCAGCATTGCCACAACTGGGGCATAAGTCTCAACTGGGGCTCAGCTCTGATCACTG
GCCCAGGAACTCCATATGCATCGGGGCAGCCAAAAAAGAAGAAAAAAAAAGTGTCTAATATGGTAATAGGAATAGAT
ACAACCCATGTAAACAAAAGTTTTTTGGGGTCTTCAATAATTTCGAAGAGTGTAAGGGGTCCTGAGACCAAAAAGAT
CAAGAACGGCTGGTCTACGTTCTAAGCAACTGCTGTGGTTCTTGTTAAGTTTTAATACTGAAGATGAGTTTTTACAA
GGACAAACAATATAATACAGGGCATGTAGCCAATATTTCGTAATAACTATAAATGGAATATAGCCTTTAAAAAGGCC
AATCATTCTGTGGCACCCTGAAATTTATATGATACATGAACTGTACCTCAATAAAAAAATTTAATAAGATAATAATA
ATATAGGTGAGCTTCAATTAGCACATTCTATTACTTATCTTTAATAAAAATTATATTCTGTGTGCAAGGTAATCTGA
CAAACTCACCAGTACAACTGGTTTCCAACATAGACCTGGCTCAGCTGCAGAGGTTCCTTTCAAGAGTAAACTTGCAG
GGCTTTCCCCGCTGTGGCACAGCAGAAATGAATCAGACTAGCATCCATGAGGATTCAGGGCACAGAAACAGCTCAGA
TTTAGTGTTGCTGTGGCTGTGGCCATGGTGTAGGCCAGCAGCTGCAGCTCCAATTCGACCCCTAGCCTGGGAACTTC
CATATGCTGATGTAGGAGAAAATGTCCCAATAAAATGTAGAAAGGAGAGACCCCGGCCATGACGACTAAGCAAAGTC
TAGCCAACTGCCCCAACCAGTCCTCCCCCATGCATCTGCTTCTGTAAATTTGTTTCCGCATCTACTACCTTGCCTGA
CGTCACTCCAGTCCAACTAGCCAAGCTTGGACCTGGAAGACGTAGCCCATAAAAGCCTTGTGAAACCCTTCTTCCGG
GCTCAGACTCTGGAGAGTGATCTCGTCTGAGCCCGCCGGCGTAATAAACCTGAGTTCTCCAACTCTCCAAGTGCTCG
CTTGGTTTCTCGCCGGGTAAAAGAGCTGCTCCACTATGGCCACAGAGCTACTGGAGCTGGTACGCTACAGCCACGGG
GCTGTCGCCAGAGCTGATACGCTGCAGCGCAGGGCTGCTGGGTATCTGCTGTAACATTTCTGGAGGCCCCAGCGAGA
TTCCAACCTTTCTGGCCCCTTGAGCCACTGGAACAGAGGTAAGGCCGCCCGGGAGCCGGGGAGCCTCAAACCGAACG
AGGCGGCGCACCACCCGACGGTATTCTGGGTCCTCCTTCGTCAGCGGCATTCCTGATTCCCGGGTGACCAAACCCTG
ACCAGACTCAGTGGAGAGATGGACCAACTCACCAGAAAGGTATCCGGACAAGGTAAGGCAGCGGGGCCAACCCCAGT
CAGGTCCTGCCCCAGTGGGCAGAAGAGGGGACTGATCACCCCCTGAGGGAGACTCTCCCGGTCAGAAGCTGTGCCTG
ACTGGAGCAGCAGTCCTAGTGCTCCAGATTGGAAGCAGAGGAACCTCTTGCTTGGGTGGAGCAACTGTCAGGTGTAG
CCAATTGAAAGTTGTGCTTGATCGAGCTACTAGTTAGGGACTCCCAGGGAGTGGGAGGCATTGTGATAACCTCTGAG
TGTGTGTGAGAGTGAATGAGCGGCCTGATTCGCTTGTGCTTCAGGTTCGAGTTTGTGGCTCCACGGTCTTAGTGGCT
ATGGAGTCTGAGTGGGTCCTAACCTGCAGTTCCGTGGTGACCTCATAGGGCTTATGGCTGCAGCAGACTCTGAGGGT
TCTGTTCCCTCCCTGCAAGTCCAATCCAAGTTCGGGGATTATACGAACCAGCCAATTGCTAAGAGGCACCTAAACTC
CCGAGAGGGGGGCAGTCAGGCGGACATCTGAATGGCCACCTTCTGAGAAGGAGGCACCCTCCCTTGTTTTGTCTGCG
ACACTGGCACAGGGCGTCCACATGGGGTGGGACCTAACCCAGAAGCCCACGAGCCAGAGACCCCTGTGCTTCCGCCA
TTTTGGGCCATAAATTCCTCCAAGGAGATGACCTAATTTGATCTTGCCCCTGGGCCTCCAGGAACTCCCGGCCCAGA
TTCTAAACCAGCCATGGGACTGCCTATTTTGTCAGTTCATGGAGGCCCAGGATCTGAGTCAGGGAGACAAGCCTGTC
ATCCCTGGCTCAGTTCAGGGTATAGGGAGGATTGGGTACAAGGTCCCCTGTCCTTTGCCCAAAACATTAGAACTTGT
CTGAGAGTGCCTTCCTGAGACCGGGGGTCCAGATGGATTGGAGATACTTGCAATAAAGCAGGTGCTCTTCCCAGTCA
TAGAGCAAGCTGAGTGGGATCTGTCTTGCTTTCAAGAGTGGTGGAGGCAAAGCTACTGGGGATACCACCCACGAGGC
CAGAAAAGGTCTCATAATATCAGGCCATAGAAAAGATCCACATAAAGACACCATGGGTTCACCCAAGTCTAAACCTG
TGGTTGTAGACTGTGTGATCAAAGATTTCAAAAAGGGATTTTCTGAAGATTATGGTATAAAACTAACCTGATCTTTC
ATCATTTCCTTTGCCATTACCTCAAATAGAGCTGTGGGGGCAAAGGAAACAGACCTCTAGATGTTAAGACCATCCTG
AGTTGTTACCAGGCCTGTGGGGGAAAAGGAGTTCATAGCTAGTATTCATCCAACTTAGGCCAAGTGTTTAGCCTCAG
AGCCTCGGCATAGTCAGTTTTGCTTTTTGCTGTTTACTTTCATCCTGGTTGGAGTAATTGATGGCTGGTTCATCCAA
TTTACCTGTTAACTGTGGTTTAGAAACTTTCCTAATGTTAATACAGGGCATGTCAGAGTGAGCATCTTAGGATTTGA
AAACTCAGGGCAGGGCCTGTATGCCTGGGTTTTCTTCACCTCTGTCCAGAGACAGGCACTGGGCAGGGATGACGGGA
AGAGAGGCTACGCTGGTAAGGAGTGGTTAATTCCAGTCAGCCTGAGGTCGGATGGGACATTTGACCACTAGTGTCTA
GCTGCTCCATATAAGAGAGGGGACACCCTCACATAGCCAAGAAAGGACAATAGGCGCTGGATGCTGTTTTTTGTCTT
TTTCGGATGGGAGCCACATCCTCAAGCCTGCTGCATGACTCAATAGCAACCCCTCTGACATGTGCCTTGAAGAACTG
GAAAAAGTTTGACCCTGAGATTCTGAAAAAGAAACATTTAATTTTCTTTCGAACAAAAGCCTGGCCGTTATATAATC
TGTCAGATGGAGAGTGACAGCCATCTGAAGGCTCACTAGCTTATAATACCATTCTCCAATTAGCCAGAAGTTAGTCA
GCCTTCTCCAATACTGCTCAAGGCTCCTTCTCCCCGCAAGCCAGTGCCAAAGTTATATCTCTCTCTACTCCCTTTAC
AAGAAGTAGCAAACAGAGAATGGAGGCCAAATACAGGTCTATATACCTATTTCACTTCAGGACTTAGGGCAAATAAA
AACAGATTTGGGAAAATTTGCTGATGACCCAGATATATTGAGGTTTTCAGGGTCTCATGCAGTCCTTTGAGTTAGCC
TTCAAGGACGTCATGTTATTACGGAAACAGACATTGACTATAAGTGGAAAATTACATAAAGTCTCCAAAACTGCTCA
AAGCTGGGGAAGATGAATGGAATGATGCTAAAAATGCCAGAGGCAGATTAGAAGAGGAATGATCAAGATTCCCCACA
GGGTGTCAGGCAGTTCCTATGAGCGATCCCAATTGGTCTGCTGATGAGGGAGATAACAACAATTGGCATAGAAATCA
TTTTATTACTTGTATAGTTAAGGGATTAAAAGCCCGTTAAAACTATCGGAGGTTTACTAGGGGAACAAGAGTCCATC
AGCTTTCTTAAAAAGGCTCAGAAAGGCATTGAGAAAACATAAAACAGGGAACCCAGAAACAATGGAGGGCCAAATAA
TTATTTATTTATTTATTTATTGTCTTTTTGCTATTTCTTTGGCCGCTCCCGTGGCATATGGAGGTTCCCAGGCTAGG
GGTCTAATCAGAGCTGTGGCCACCAGCCTACACCAGAGCCACAGCAATGCAGGATCCGAGCCGAGTCTGCAATCTAC
ACCACAGCTCACGGCAATGCCGGATCGTTAACCCACTGAGCAAGGGCAGGGATCGAACCCTCAACCTCATGGTTCCT
AGTCGGATTCGTTAACCACTGCGCCATGACGGGAACTCCCGAATAATTCTTAAGGATAAATTCATAGCTCAATTGGT
GCCAGATATATGGAGAAAGCTCCAAAAATTGGCTTTTGGCCCTGATCAGGACCTGGAGCACCTCCTCAGAGTAGCAA
CTCAAGTATGTTATAATCTGGGCCAGGAAGAATAAAAGGAGAATGAGAGGAGAGACAGAGAAAAGGCTGAGGCTCTA
GTTATGGCACTACAGGGAGTCAACCTGGAAGTTGCCAAGGTGAGAGGACTAGGGCAGAGACCTATGCCTGCAGCCTG
TTTCCTCTGTGGAAAAGAGGGACCCTTTAAATGGGAATGCCCCAAGCCTCAGACCACAGCACCTAGGCCATGCCCCA
TATGTTGGGGAGATCACTGGAAGAGGGACTGCCCCTGAAGATGAAGGTCTCTGGGGTTGACCCCTCAGGCCCAGGAT
CAAGGCTGACAGGACATTTCCATAATGGCTCCTGTCCTTCTCACCACTCAGGAGTCCTGGGTGACTCTAAATGTAGG
AAGACAGCCTATTGACTTCCTCCTGAATACGGGAGCCACTTTTCAGTCCTCCTCTCCAATCCTGGGCCCCTCCCTCA
TGAATCTGCCACATTTATATTTCCGGCAAGCCGGTTACAAAATTTCTTACACAGCCTTTGAGTTGTGGCTGGGAATC
CATTTTCTTCTCTCATGCCTTTCTGATTGTTCCAGAGAGTCCAACTCCTCTTTTAGAAAGAGATATTTTGTAAGAGG
TTAAAGCCTCAATTCACATGGCAATGGAGCCTAATCAAGGTTTATGCCTGCCTTGGATGGAAGTATATACTGACCCA
GAAGTCTGGGCCATAGGAGGAAACATAGGAAGAGAAAAGAATACTCAACTGGTGGAAATAGGTCTTAAAGACTGGAA
TTTATTTCTTTGCCAAAAGCAGTATCCTCTGAGACCCAAGGCATGACAGGGACTTGTATCAATTATAGGAAGCGTAA
GAGAACAGATTATTAATTGACTGTATCAGCCCTTGTAACACTCCTATATTGGGAGTGCAAAAACTTAACAGGGATTG
GTTCCTAGTACAAGACCTCCATCTAATAAATGAGACACTGGTCTCATTACATCCAGTGGTGCCCAATCTCTACACTC
TTCTTTCACAAATTCCAGAAACAGCAGCATGGGTTACTGTATCATATTTAAAAGATGCCTTTATTCTGCATTTCCTT
GACTAAGGCTTTGCATATATAAATTCTCAAAATATGGAAGGTAACTAACTGACCAGAATTAATTTTAGGTTCAAGTC
AACTGGGAAATATTCAGTATTAAATTAATATCTTAAATTAGAATTGAAGTTTGCTGATCTAATTAATACACACATGT
CGTTACAGCTGTCAACATTAGGTATAATATCTTATCGTACCTAGGTTTAACAGAAGTCAAATGAGACACTGAGACAT
CAGTTACTAAACAGAAACTAAAGGTATTTAGAATAATTAATCAATATGATCAGTTTCACCCTGAATGGTCTCCATAA
GAAAAACATGTGTTTTTAGAAATTATAAAGGACAGTCTGTGGTTGCTTTAGAAACGTAGAATCTGTGTGCTTTCAAT
ATAGAAGGAATGAGGGATGGAACTGCATTTTATGAAGGCAAAAGAAAGTCTGTCTTCAGCTGATTGCTCTGGTTGGA
AAATAAGGGACAGACTAATATGGATACAGAAAGTGATACAAGGTGTGTGGGAAGTGGACACTGAGAATTTTGTGCAT
GGTGGGGACTGTCTATATTTGAGTAAGTTAACTTTAAAAGTAATGTGGTGCCATAAATCATACTGCTCACAAGGACA
TAAGGTAGCTTTCAATTACATGTTGACCAAGGCATACAAGTGTTTCATAACCAGCCAGAGAAATCAGAAAAATCATA
CAAGTTACCTGTGCTATTATAAAATCTAAATGTTGTATTCTTGATGGTTCACAGAATGTGTCTAATTCCCTGCTAGA
TCTTCAACAGTAGATTCATGAGCGGTCCTATCCAGCTCCAGCTTTTGGAGCTGCCCTGTGGAACCAGCCGACCTCCT
CCTCCTGGTGAAAATATTTCTTCACCATATCTTTTTATTCAGACCCTGTATAATTAACTGTATTTCTTGCTTCATTA
CATCCTGATTAAAAGCCATCAGCCTTAAAATGTTGATAGAAGGGGTACCCAAAGCAATGTATCAAAGCCCACTTGAC
CGTCCCATGAGTGGAGACCTAACTGCTTTCCCTAATGACGCCCCTTTTCAGCAGGAAGAAGTCAGAGCGGTCATCGC
CCCCTTTCCCCACAGTTAGAGTCTCTAACTCACTGGTGGGATTGAGGCAGAATATTCACTCAGGTAGTCAGTGTAGG
AACATGGGCTTCGATACATTCTTTGATGTGGCTATTGGTTAACATTTGTAAAGTAAGGGTTGCACAGCAACCCCAAC
TGCTATAAAGGTTACAGGTATTACCCCATGGATCCATCACACCGGAATAAAGAAGGCTGCTCCCGCCATTGACACAG
ACACCTGGGAAGCTGTCCGGCACCCTGAGAACCCCCCTCAGGATCAAGTTCCAGAGACATATGGCACTGGAGGATGG
CAGGCCCTGCTCTGGTCACACCCAGAAGCTGGCCAGTCTATGCACGGCAGAAACTTGAGGAGTCTACAGCCCTGCCC
CAGCCACATACTGGAGTTGGTTGGTTTGTACAAGTGGAGGCCAGAGGATCTCTATGCAAACTTGAATTGAACTCATG
CTCTGGTGGGGAATATTGGTAATTGAAATTGCCATAGCCCTCATATTTGGAGTGGGGCTATATGCAGTATCCCCTTC
AGAATGGGGACAGGGAGCCCAGCTACTCATCTGTGTGATGTATCTCCTGACTGTCAGTATACTAGAATCCCTGTTCA
TAATGGGTCAGTGAAAAGGATCAAAGGAATCATAGTTCTGTTAACACTCACCCTGCTGCTCACTCCAGGGGCAACAG
ACTGGGACAATGATCTATGGGATGGGACGGGATTAACAGATGCTTACCAGTGCCTCCCTGCTAATTGGACAGGGACC
TGCACTCTAGCCTTTGTCACTCTTCAAATAGATATTGTCCCTGGGAATCAGTCTCTTATGGTGCCCATAGAGGCACA
TGGCAGAACAAGACAGCAATGCAAGTTATCCCCTTATTTAGTTGGTTTGGGAATTCCAGCAGGGATAGGAGCAGGAG
TGGGAGGAATAGAATCCTCCACTGCTTATTATCATCAATTATCTAAAGAATTCACGGATGATGTGGAACAAGTAGCC
CCTTCCCTAGTAGCCTTACAGGATTAGGTAGACTCTCTGGCAGAAGTGGCCCTTCAAGACAGGAGAGCACTGGACTT
ATTCACTGCTGAAAAAGGGGAACTTTGCCTGATGAAGAATGCTGTCTTTATGCCAGCAGATCTGGAATAGTCAGAAA
CATGGCCCAACAAATAAAAGAACGCATAGCAAAGAGAAGGGAAGACTTAGATAACTCCTGGTTAAATTGGAGCAACT
ACTGGAGTTGGGTGGCATGGCTCACGCTTTGGTTGGGCCCCTCCTCATGCTCTTCATGGCCCTCACATTTGGCCCCT
GTATCCTGAACTGTCTTGTCAAGTTTGTCTCCTCAGGCCTAGAATCTATAAAGCTACAAACGGTGGTGATGTCCCGG
CCACACTTATATCAGCCTCTGGGCCAAGAAGACCAGAAAGGTTGATGCTTGCTCCAAGAATGTGAAAAAGCATCAAG
AGGGGGGGATGTAGGAGAAAATGTCCCAATAAAATGTGGAAAGGAGAGACCCCGGCCATGACGACTAAGCAAAGTCT
AGCCAACTGCCCCAACCAGTCCTCCCCCATGCATCTGCTTCTGTAAATTTGTTTCCGCATCTACTACCTTGCCTGAC
GTCACTCCAGTCCAACTACCCAAGCTTGGACCTGGAAGACGTAGCCCATAAAAGCCTTGTGAAACCCTTCTTCCAGG
CTCAGACTCTGGAGAGTGATCTCATCTGAGCCCGCCGGCGTAATAAACCTGAGTTCTCCAACTCTCCAAGTGCTTGC
TTGGTTTCTCGCCGGGTAAAAGAGCTGCTCCACTATGGCCACAGAGCTACTGGAGCTGGTACGCTACAGCCACGGGG
CTGTCGCCAGAGCTGATACGCTGCAGCGCAGGGCTGCTGGGTATCTGCTGTAACACTGAGGGTGCAGCCCGAAATGG
TAAAAAAAAGAAAGAAAAGAAAAAAAAAATAGTAAACTTGCAACCACAGTAAGTATATAACGGAGTTCCTGTCATGG
CTCAGCAGGAAAGAATCCAAGTAGGAACCATGAGGTTGGGGGTTCGATCCCTGGCCTCGCTCAGTGGGTTAAGGGTC
CAGTGTTGCCGTGAACTGTGGTGTAGGTCGCAGACATGGCTTGGATCTGACATTACTGTGGCTGTGGTGTAGGTCAG
AGGCTACAGTCCCAATTAGACCCCTAGCCTGGGAACCTCCATATGTCGCGGGAGCGGCCCTAAAAGGACAAAAAGAC
CAAAGGGAAAAAAAAAAGAATGTATATATATGTATGAGTGAGTCACTTGGCTGTACAGCATAAATTGGCACAACACT
GTAAATCAACTATACTTTAACTTTTCAAAAAGATTAAAAAAGAAGCATTGGCGTTATCCTCAAGTACAGCTGGATTC
CCATCTGCTCCTTATAATGCTGCCCTTGGGCAACCTCCATTCTCCATGTTCACAGCTCTGAAGTGGACATAACTCTT
CCAAGAGTGTTGCTGGGCGCATTAGAGGCACAATCTAGAACAGGGCCTGTACGTAACAGATAAGTGCTCCACAGTGG
ATGAAATGAAATGAATTCACCAACAGGAAGTAACGATCATTTCCTGGGTTGGTAGGGTGTGTTGTAGTGAAACATCC
TTTCTCAGAGGGACAAAGATCAGAAATGCACATTTCAAAATCAGACACTCTTTAATTTAAAAAAAAAAAAAGAAAGA
AAGAAAAGAAAACGAAAAAGGCAAATAAACATTTAAAAGAGTAAGTTTCTTCTGAGGAAGAAACCTGTTTCCCAAGG
TCACCCAAGCCAGCAGCCTTAAAATCTTAGAGACATAAACACAGCAACATGGACTTGCCAGAATGTTCGGTTGGCAC
CAGTTTGGATCCTGGTATCAAGACTCCTGGTCATTCTCCTCATTCACTAAGGAATGTGGGATGAGATAATTTTGGGG
AAGTGCTGGAAGGAAAGCCTTAGAAGGGACTTTAGCTGGTAACGCAAGAGCTACCTCCCTTTGCTGAGTTCTGCCAT
AGCCTCAGTACAAACGTGTTTCTTGGTTTCCTTATTTGTTTCGGCAGCGCCAGGGCATGAGGAAGTTCCCCGGGTGG
CCAAGGATCAAACCCTTGCCACAGGAGGAAAAACGCTGGATCCTTAACCTGCTGCACCATCAGAGAACTCGTATACT
TCATTTTAATCCTCATAAAACATCATCTAACCAACACGGTTCCCCCCCTCCCCTTTTTTAAGCCATTTAGGGCCGCA
GGTGCCTGTGTATGGAGGTTCCCAGGCTGGAGGTCTAATTGAAGCTGTAGCCATCGGCCTACACCAGAGCCACAGCA
ACGCGGGATCCGAGCCACGTCTGCGACCTACACCACAGCTCACGGTGACACCGGATCCTTCACCCACTGAGCAAGGC
CAGGGATGGAACTTGCAACCTCATAGTTCGTAGTCGGATTCGTTACCCACTGAGCCACGACGGGAACTCCCACAAGA
CGTATTTCTGATCCTTCTTTCTGTTTATAAAAATTAAATGAGCTCACCAAGTCCGCACTTCCTCCGTTAATTATTAT
GCTACTCAGAAGTTTTTTTTAGCACCCCAAACCACAAAACGGACGCTCGCTCCACCGCGAGGCTGTCTTCCGGAGCA
GAAAACTGACCTTTTAAAATTTTTTTTTCTTTTGGTCTTTTTGGGGCCGTACCCTAGGGCATATGTAAGTTCCCAGG
CTAGGAGGTCTAACCAGAACTGCAGCCGCCGGCCTTACGCTGCAACTAGATGCTACGCCAGGTCCGAGTGCGTCTGC
GACCTACACCACAGCTCACAGCAACATACCCACTGAGCGAGGCAAGGGATCGAACCCGCGTCCTCGTGGATACGGGG
GGCGGGGAGGGGCGTAAACCGTTGAGCTAGAACAGGAACTCCTAGAAAACCGACTTCTTCAAAAACTCTGCCTCTAA
AACCCCCAAGCTGTTATTTAATGCAGCGTAAAGGACGCAGCCTCCGCTTCCCCACAGCCTGGGGCCCCACAGCCTGG
GGCCCGCACATCCCCCGAGACTTACATCCCCAGCCCTGGTCATAACCTCCGAGTTCCGGGCCGCCCCCCGTGCTCTG
CGCCACGAGAGGCAACCTCCACGTCGAATGTTCCCCTGGAAAACCAGTGTTCCTTGGGGCGCAGGGCGGGGGAACGA
GCAGGAACTCTCAACAGCGTCCCGAGGCGCAGTCTCCTTCTCGCTGTCTCACCGACGTACGGAGCCGGTCGGACTTA
TTTTGGAGACCCGCCGCCCCCCCTACTCGGCTCCGGGGTCCCGGGACCTGGCCGCTCCCGGGTGGCGCCACTGGCTG
GCCAAGTTTGACTTCCCATTTGTCTCTGCTCGAGGGACACGCACCTGTACGAAGTCATCCTTAATCCCGCCGCCTCG
GGACATTCTGGGCTGGTGGTGCCACTCCGCGGATTGGACAGCCCTAGCACCAACCCCGGCAAATTCTTCCTGGTAAA
CCGCGAGAGCTTGGGTCGGACCCGCCCACGTCACCACCAACCCCCGC

| | |
|---|---|
| SEQ ID NO: 24 | B4GALNT2 cDNA Sequence |

TCCGCGGAGTGGCACCACCAGCCCAGAATGTTCCGAGGCGGCGGGATTAAGGATGACTTCGTACAGCCCTAGATGTC
TGTCGATCCTCAAGATATTGATGGTGCTTTTGGTCCTGAGCGTTGGACTCTTTATGTTCCAAAGCGTGTTCCTCGAT
ACAGACTTCAGTCTCCTCAACTCACCCATCCCGTCCCCCACCCTGGATGCGCAGACGCTGAAGCTTCTACCTGAGAA
ACCCGATTTCTACGGTGAAAACGGGCTGTTCCCGAAAAACCAGTGCCAATGTGACGCCTTCGGGCATCAGGAAAGCT
ATAACTTGGAGGATGCCTACGACCCGCAAGACCTCCCCGCAGTGAACCTGAGGAGACAGGCTGAGCTCGAACACTTT
CAGAGGAGAGAAGGGCTCCCTCGCCCACCGCCCCTGCTGGCTCAGCCCAACCTCCCCTTTGGGTACCCGGTCCACGG
GGTGGAAGTGATGCCTCTACACACCATCCCCATCCCAGGCCTCCGGTTTGAAGGACCTGATGCTCCCATCTATGAGG
TCACCCTGACAGCTTCTCTGGGGACACTGAACACCCTTGCTGACGTCCCAGACAATGTGGTGAAGGGCAGAGGCCAG
AAGCAGCTGAACATTTTGACCAGTAGCCGGGAGCTTTTGAATTTCATCCTCCAGCATGTGACATACACGAGCACAGA
GTACCACCTCCACAGAGTGGATGTGGTGAGTCTGGAGTCCAAGTCCTCAGTGGCCAAGTTTCCAGTGACCATCCGCT
ATCCTGTCATGCCCAAGTTATATGACCCTGGACCAGAGAGGAAGCTCCGAGACCTGGTGACCATTGCCACCAAAACC
TTCCTCCGTCCCCACAAGCTCATGACCATGCTCCGGAGTGTTCGTGAGTACTACCCAGACCTGACGGTGATCGTGGC
CGATGACAGCAAGGAGCCCCTGAAAATCACTGACAGCCACGTGGAGTATTACACCATGCCATTTGGGAAGGGCTGGT
TTGCTGGCAGGAACCTGGCCATATCTCAGGTCACCACCAAATATGTGCTCTGGGTGGACGATGACTTCATCTTCAAC
AGCAAGACCAGGATCGAGGCGCTGGTGGACGTCCTAGAGAAAACGGAACTGGACGTGGTAGGTGGCAGCGTGATTGA
AAACACATTCCAGTTCAAGCTGTTGCTGGAGCAGGGGAAGAATGGCGACTGTCTCCACCAGCAGCCAGGATTTTTCC
GGCCCGTGGATGGCTTCCCCGACTGCGTGGTGACCAGTGGTGTTGTCAACTTCTTCCTGGCTCACACAGAGCGACTC
CAAAGAATTGGCTTCGACCCCCGGCTGCAGCGAGTGGCTCACTCAGAGTTCTTTATTGATGGGCTCGGGAGCCTGCT
CGTGGGGTCCTGCCCACACGTGATCATAGGTCACCAGCCCCATTTACCAGTGATGGACCCAGAGCTGGCCACCCTGG
AGGGGAACTACACCAGTTATCGGGCCAACACCGAAGCCCAGATCAAATTCAAGTTGGCTCTCCACTACTTCAAGAAC
TATCTCCAATGTGTCACCTAAGGTATCCGGGCATTGGAAAAGCGCTGAGCTGCCTGGTTGCAAGTATCTAAGACAGC
GGATGCGGTGGCTGGGATACCAATATTTGAACTCCTCATAAGATAAGCACTGTAATGCCCAGGGAGCAGGGTAGGCA
GGTGGGTCTGACTCCGTTACTGGAAGTACCAATAAAAGTACAGGGTCATTAGAAATGGACCAGTCACTGAGGTGGGC
AATGGAGACTTCATTCATAACGATTACGGCGGTGTTTCCATCATGGCTCAGAGGTAGCAATCCAGACTGCTATCCAC
GAAGATGCGAGTTGGATCCCTGGCCTTGCTCAGTGGGCTAAGGATCTGGCATTGCTGTGGCTGTGGCATAGGCTGGC
AGCTGCAGCTCTGATGCGCCCCCTAGCCTGGGAACTTCCAGATGCTAAGTGTGTGGCCATAAAAAAAAAAAAAAAAA
AAAAAAAAAA

| | |
|---|---|
| SEQ ID NO: 25 | B4GALNT2 Protein Sequence |

MTSYSPRCLSILKILMVLLVLSVGLFMFQSVFLDTDFSLLNSPIPSPTLDAQTLKLLPEKPDFYGENGLFPKNQCQC
DAFGHQESYNLEDAYDPQDLPAVNLRRQAELEHFQRREGLPRPPPLLAQPNLPFGYPVHGVEVMPLHTIPIPGLRFE
GPDAPIYEVTLTASLGTLNTLADVPDNVVKGRGQKQLNILTSSRELLNFILQHVTYTSTEYHLHRVDVVSLESKSSV
AKFPVTIRYPVMPKLYDPGPERKLRDLVTIATKTFLRPHKLMTMLRSVREYYPDLTVIVADDSKEPLKITDSHVEYY
TMPFGKGWFAGRNLAISQVTTKYVLWVDDDFIFNSKTRIEALVDVLEKTELDVVGGSVIENTFQFKLLLEQGKNGDC
LHQQPGFFRPVDGFPDCVVTSGVVNFFLAHTERLQRIGFDPRLQRVAHSEFFIDGLGSLLVGSCPHVIIGHQPHLPV
MDPELATLEGNYTSYRANTEAQIKFKLALHYFKNYLQCVT

| | |
|---|---|
| SEQ ID NO: 26 | C3 Genomic Sequence |

CTCACTTCCCCCCCCACCCCCGTCCTTTCCCTCTGTCCCTTTGTCCCTCCACCGTCCCTCCATCATGGGGTCCACCT
CGGGTCCCAGGCTGCTGCTGCTGCTCCTGACCAGCCTCCCCCTAGCCCTGGGGGATCCCATGTGAGTAATCACAACC
CCAACCCCCAAACAAGGCTGCTTCTGCATTGGGAGTGGGCACTTGTGAGTATAGGTCTCTGCAGGTTTAGGGTGCAT
GTACGGTGCTGGTTGATTCTGTGGCTTGTGATGAGGTTGGGGTGAGTCTCAGAAGTTGGGGTTGGGTGAGTCTCAGA
AGTTTGGACTCCATAGGATCTGGGAGTTTGTAGTTTTAGCATTTAGGAGTTTCAGAGATGCGGTTTGGATGTATGTG
GCTGAGGGGATGGATTGGGTTGTATTTATAGGTCTGGGGTGCTAGAGGTTTAGGAGGCTGTTTAGGGTGTTCCAGGG
TTTGGGTATTTAGAGACTTGAGGTATTTAAAGATTTAGGAGTTCTGACCTTGGAGCAGTGGGTTAAGAATTCGACTG
CAGAGGCCAGGGTCGCTGATCCGGTGCGACCATAAAATGATAAAAAATAAATAAACGATTAAAAAAAAGATTGAAGG
GTTGAGACTTCTGGAATTTGTGGGTTTGATTGTGGGCTTGGAAGTCCATCGTCTTGGAGGAATTGGTTCTGATTTTG
AGGTTCAGGAATTGATGGGATCTGAAGCCCCCAAGCTGTCCTCCAGTCATCGGATCCCCCGCAGGGCTAGGGGCTGG
GGCAGAGCGCTGACCCTGGGGGTGCCTAGCATCTCGTGCCCCTGGGATGACAGCTCTACGCCTCGTCCTCCCCTCCC
GCAGTTACACCATAATCACCCCCAACGTCCTGCGTCTGGAGAGTGAGGAGATGGTGGTGTTGGAGGCCCACGAAGGG
CAAGGGGATATTCGGGTTTCGGTCACCGTCCATGACTTCCCGGCCAAGAGACAGGTGCTGTCCAGCGAGACCACGAC
GCTGAACAACGCCAACAACTACCTGAGCACCGTCAACATCAAGGTGGGCGCGCTCAACAGCCGGACCGCTGAAGCCC
CACCCCTTCTTTGAGTCCTCTTGGTAGCTGAGCCCCTCCTCCCTTTCTGAGCCCCACCCACCCTGCCTGAGCCCCGC
CCCTTCTGTCTGAGTGTCTCCATTCTGAACCCCGCCCCTCTGAGTCTCCTCCCCTTCGGAGCCCTTCCCCTTTTGGA
GTCCGGGTCACTTTTTGGAGCCCCCTCCCACTCTCTCATCCCGGTCTTTCTCTGAGTGTCCCCACCTTCTGAGCCCT
CGTCTTTCTCTCAGCCCGGCCCCCTTCCAAGCCCCACCATGTCTGAGCCCTTCCCCATTTCTGACCCCTCCCCTCCA
ACCCTCCTCCCTAAGTCCTTTCTTCTTTTAGAACCCGTCCCCTCTCCGAGTCTCCTCCCCTTTCTGAACCCCCTACC
CCTTCTGAGCCCTCCTTCCGCTAAGCCCCCTGCCTGAATCCCCCTTCCCATCCCTCCCTCTGACTCCCTACCCCCTC
TCTTGCCCTTTGGCCCTTCCCCGAGTACCTCTTCTCTCCCCAAACCTGGGCAAAGCAGGAGGACCAGAAGTGACAAG
CAGGCTCTGTTGCGAGGAGGGGCGGGTGCGGACCCAGCCGAAGTCCTAGAGGCTGGATGGTGGGCAAGGGGTCTTGG
CCCCTAGTGATCCCCTGGTTCCTGCTCAGATCCCGGCCAGCAAGGAGTTCAAATCAGAGAAGGGGCACAAGTTCGTG
ACCGTTCAGGCGCTCTTTGGGAACGTCCAGGTGGAGAAGGTGGTGCTGGTCAGCCTTCAGAGCGGGTACCTCTTCAT
CCAGACGGACAAGACTATCTACACCCCAGGCTCCACGGGTAAGGGGCTGAGGGTGGCTGCAGAGAGCCAGGGGCAGG
GCTGGAGGAAGGGGCAGGGCCTCACCCGGCTCTGCTTTTCTCTCCCACCACTGCTCAGTCCTCTATCGGATCTTCAC
CGTTGACCACAAGCTGCTGCCCGTGGGCCAGACCATTGTCGTCACCATTGAGGTACCAGCCGACTGGGGCCCCAGAC
ATACCCAGGGCAGGGACTCGGGGAGAGACAAAGAGAGAGAGAGAAACAGAGAAAGGGATTCCGGCAAAGGCCCAGCA
GCAGAGACATAAAGGCAAAAAACAAAACCCCAAAAACGTAAGGGCACACAGAGAGATCGGGAGAGAGGCGGGGACCC
AGCGATGCTTACCGTGGATGACGGCTCCAGATAAGTCCCTGGTCACTGTGTGAATCTGGACAGGTCACTTCATCTTT
CCAAGCCTCAGTTTCCTCATTTGAAGACTGACACGACAGGTACTAATTCTATGTAGTCTGTTCCGCCTACTGCCCGC
CAGAGGGCGCGTGGGAGCACCTGAGTCAGGTTCCACCCCTCCTCTGCCTGCCGTTTTCCAGGGCTCCCCGCTCCTGG
GGTAAATGCCCAAGTCCTCCCCACGGGCCTCAAGGCCCTGCAAGACCTGCTCCCGCACCCTGCCCACCCTCCTTTCT
TCCCTCTCTCTTCCTCCCTCCGCTCCAGCCACGTGGGCCTCGTCACCGTTCTTGCAACAATCCAGGCACAGTCCTGC
CCCAAGACCTTTGCAGGGGTTGTTCCCCCTCCCCCCCAAATGCTCTTCCTGCAAATATCCACACAGTTTGCTCCCTC
ACCTCCTTCAAGTCTTTGCTCAAATGTCACCAGTGTACCAATTTTACAGTGAGGCTTGTCAGAGCGCCCTGTAAAAT
TGCAACAGAACACACACACACACACACACACACACACACACACACACACTCCCTTTTTTGCCTTCCTGCCATCTCTT
TTTGGCATCTTATAAATCGGAGTTATTTCCCCCCTCCCTTTTTTGGTCTTTTTATCTTTTTAGGGCCGCACCCGCAG
CATATGGAAGTTCCCAGGCTAGGGGTCGATTTGGCCTAGGCCACAGCAATGTGGGATCTGAGTTGCACAGCTCACAG
CAACGCAGGATCCTTAACCCAGGGAGCGAGGCCAGGGTTCAAACCCAAGTCCTCATGGATACTTGTTGGGTTCGTTA
ACCACTGAAGCACGATGGGAAGTTTTTTGGGGTTTTTTTTTGTGGGACCTATTCCTTTGTTAACTGCGCCTTCCCCC
AATCTGCACTGAACCTAAGTTCTGTTCAGAAAGGGATTATCTGTTGGCCCAGAGTTTGGCGGGTAGTAGGGTAAATA
AAAACTTACTGGAAGAAGGGAGGGAGGGAAGGAGAGGGGAGTGAGAAGCAGGGAGTGATGGGGAGAGAAAGACAAGT
GGAGGAGGAAGGGGAGGAATGGGGCCTGTCCTCCTTGTGGGATCTTTGTATTTATTGAAATCAGGCAAACCTAACAA
GGACCAGAGTTTTTGTGTGTGTGTGGTATCAGTATGTGTGTGGGGTTTTTTTGGTTTTTGTTTGTTTGTTTTTTGCT
TTTTAGGGCCATACCCTCAGCATATGGAGGTTCCCAGGCTTAGGGTCCAATCAGAGCTACAGCTGCTGGTCTACACC
ACAGCCACAGAAAGGCAGGATCCAAACCACATCTGCGACCTACACCGCAGCTCACAGCAATGCCGGATCCTTAATGC
CGGACTGAACATGCAACCTCATGGTTCCTAGTTGGATTCGTTTCCACTGCACTACGATGGGAACTCCAAGGAGCGGG
TTCTGAAGGCTGTGTGCTCACTTTAGTGATGGTGGAAAACAGAGAACACCCTCCTCTAAAGATGTGGCGCTGCCAGA
CTCCCATTGAACGTCACCTCATGCCATTGGGAAGAACATATCCACAATTACCTCCACTTGCCAGAGAAGCTAGAGAA
TCAGATTTCTCTTTGAAGTCTCCTGATGTTTAGCTATTGGCAACAAATGAAATCATATACTTATTAGGTTGAGCCAC
ACGAAGTTGCTATTCTTGCAGGTCAAAAAGGTGAATGTAGGCAGTGATGTGTGCCTTCTACAAATCAAATGCTCAGC
CCAGGGTCCTATATCAAAGGAGGTGATAAATTCTAGTAATTACTAGTCTTCAGAGCGACACAGATCATCACAAGCAC
TTGCCTACACTAACAGGTCCCAAACCAGTGACACAGGAGCTGTAGTTATCTCCTTTTTCCAAGAGGTTCACATTGAG
CACAAAGAGGTTAAGTAATTTGCCCAAGATCACACAGGCTTGTAAGTGGTGCAGTGGGGACAGGAACCCAGGCTACC
TGGTTTGGGTGCCCATTCTTAACCACTGCCCCTGTAGACACGACACAGAGGAGAACCAAGGGGCTAAGCCTGGTCTC
TGAAGAGCCACTTCCCTTCCTGTCTCCTCACAGACCCCTGAAGGCATTGACATCAAACGGGACTCCCTGTCATCCCA
CAACCAGTTTGGCATCTTGGCTTTGTCTTGGAACATCCCAGAGCTGGTCAAGTAGGTCGGGCCCTCCAGCAGGGGTG
GGGTGGAGTGGTCGTGTGTTTTAGGGCTCCCCAGGAGAGGGAGTGGGGGGGCTGCCAGACCTGGCGGACTCACTAGC
CTGCCTCCCCCACAGCATGGGGCAGTGGAAGATCCGAGCCCACTATGAGGATGCTCCCCAGCAAGTCTTCTCTGCTG
AGTTTGAGGTGAAGGAATATGGTAAGAAGAGGAGGGAGCTGGGGGGGGGGGGGCGTGCATAATGTTGGACCCAGCGT
TGACCCCCCCCACCGAACGAATACCATCTGCTCCCCCCCAATAGTGCTGCCCAGTTTTGAGGTCCAAGTGGAGCCTT
CAGAGAAATTCTACTACATCGATGACCCAAATGGCCTAACTGTCAACATCATTGCCAGGTGAGGGTCTAGGGGGAGG
GCCTGGGGAGAGGGAAGGTCAAGGGATAGGGCAGGGATGGAGGGGGAGGGGCTCGTCACGGCCAGTGGACATTTGGG
GGAAGACTCCTCTTTTCAGGACCGGGGGAGTCTGAGACCCCTTCCCACTTTGCAGGTTCTTGTACGGGGAGAGTGTG
GATGGAACAGCTTTCGTCATCTTTGGGGTCCAGGACGGTGACCAGAGGATTTCATTGTCTCAGTCCCTCACCCGTGT
TCCGGTACCTAACAGTGGCCCCCTCTGAGTAACTCTTCCTCTCCCCCTCGGAAGCCCTTCCCCTCCCTGAGCCCTCG
CTTTCTCCCCCAGATCATTGATGGGACGGGGGAAGCCACGCTGAGCCAAGGGGTCTTGCTGAATGGAGTACATTATT
CCAGTGTCAATGACTTGGTGGGAAAATCCATATATGTATCTGTCACTGTCATTCTGAACTCAGGTGAGGCCCGATCT
GAGGGCGGAGGCTCCGTACCACCATGTGGTCCAGCCTGAGAGGGGCAGCTCAGTGGAGGGGAGAGGATCAGAATGAA
GGGCGACCCAGTCTGGTGGGGGGCGGTGTGTCCAGTCTGAGGGAGGAGGTCCAGAATGAAGGCAGGGTCGGGTCTGA
CAGGGGAGACCTAGGCTGGGACACAAACCCAGTCTGAGGGGGGAGGCCCAGTCAGAGGGGGGAGGCCCAAAATCAAG
GTGGGATCCAGTTCATGGGGGAGACCTAGTCTGAGGAAGGTGGGGTCCGTGTTGAGGAGGGCAGTCTGGCCCTCCCT
CATGGCTGGCCCCCCTCAGGCAGCGACATGGTGGAGGCAGAGCGCACCGGGATCCCCATCGTGACCTCCCCCTATCA
GATCCACTTCACCAAGACCCCCAAGTTCTTCAAACCCGCCATCCTTCGACCTCANNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNN
AGCTGTGGTGTAGGTTGCAGACTCAGCTTAGATCTGGCATTGCTGTGGCTGTGGTGTAGGCCAGAGGCTACAGCTCT
GATTTGACCCTTAGCCTAGGAAACTCCATATGCAGTGGGTGTGGCCCTAAAAAAAAAAAAAAAGTTTTCCCTCCTGC
ACCAGCTCCAACACCCCAAATAGTTTGGTGTGTGTTTTCTAGAAAAAAAAAGATACAGGCAGACCTCGGAGTCAGTT
CCTGGCCATGTTAATAAAGCAAGTCACATAAATTTTTTAGTTTCCTAGTACATATAAAAGTTATGTTTACACTATGC
TATATTCTATTAACTGTGCAACTGCATTGTTTAAAAAAATGTACATACCTTTATTTTAAAATACTTGATTGCTATCA
GAGTTTCCCAGCGGCTCAGCAGATTAAGAATCCAGTATTGTCACTGCTGTGACTCTGGTTACTGCTGTTGATGGGGG
TTCAATCCCCTGGCCTGGAACTTCTGCATGCCGTGGGCATGGCCAAAAAATAAAAGAAGAAAAAAAATTTAAAAATT
AAAAAATGCTTTACTGCTATCAACTATACTTCAAAGAAAAAATTGCTAGAGTAAAAAATAAATGCTTTATTGCTAAC
AAAAGTTAACCATCCTCTGATAACGCAGAGGTCACAAGCCTTTGATTTGTTTTTCAAAAATGCAGTATCTGCAAAAC
TCAATAAACTGAGGTATGCCTGCATTCTCCTACAAACCCACAGTGCAGTCATTAGAATTAGGACGTCAACATTAATT
CATTACTACCCTCAAATCCTCCATCACCATTCAAATTTTGCCAGGGTTTTGTTTTGTTTTGTTTTTTGGTGTTTGGG
GTTTTGAGGTTTTGTTTTTGTTTTTGTCGTTTATAGGGAAAGGATCCTGTCCAGAATCACAGGCTGTGTTTTCTGGT
TGGGTCTCTTCAGTGTCCTTGGACCTGTCTGACCTTTAGAGCACTTTCTTCTTTCTGTGACTTTCACATCCTTGATG
GATACGAAGTACACAGACTGAGATCTTGGGGACTGTCCCACCATCTGGGTCTGCCTGATGCTCCTTCATGACAGCAC
TCAGGTTTTGCATTTTTGGCAGGACTGTCACGGAAGAGACATCGTGTCCTTCTTGGTGCACCATTTCAGGTGACAAA
GGGTACTGATTTATCCCACTCTTTGGTGATGTGTACCCTGATTGCCTGATTAAGCTAATGTCTGCCGGGTCTCTCCA
TTGTAAATGTCCTCTTTATTCCTTTTTAGTTATTTTTAAAAACTTCTCTTTAACTATCAGATAGTGGCAAAATTCAA
GTCAAGAGAGATTTCCCTCCAAATCAGTGTTCACTTAGCCTTTAAGACAACAGGGGTGGATTCCTTATATTGTAATG
TATGATTTTCAAACACAACCGTACTTTTTTTTTCTTTTCTTTCTTCCTTCCTCCCTCCTTTCATCCCTTCATTCTTC
CTTCCTTTCTTCTCTTTTTCTTTCCTTCCTTTTTTTTTTTCCTTACAAAAAAGCACCCACCTCTCAAAGGCAGCCAT
TGATTGCCAAAATGGGCAAACATTTCTAAATTCCTGTAGTGGAAAGCTAGCAGCCCCTGCAGCCCTCCAAAAAGAAA
AAGATTCCCAATACACATGAGCAAAGGATCTTCAGTCTCTTTGCACTTTATAACTAGGCGTGCTGCTTTCTGCTCCA
GTGACCCAAGATGTTCTTTTGCAAAGAGGAACGTTTTTTTGCAAGGAGGAAATTTAGACAAAACATCTGATTTAGAG
GGGTACAGTTTACACATACGTGGATTTTTTTCAACATTGTGTCATTACTTTAACCAGTTGGGGGTGAGCCAGAGGAT
TGATTAAAAGTCAGTACCCCAAAGGCACTTTGATGGATTATTCCAGAGCGCAGATGGATTTAGGCATCTCTGGAATT
CCACCTACTTGGTTGTAAGGCAGACCCAGAGCCAAAATAAAATCTGTTCATCATTTTTTTGAGGAAAGCCCAGCCAG
GGTTGAACTCTGTTCCCGCCCAGCTTGCTGATGGTGTCAAGCTGGCTTTTAAAGGCCACCTCCTCTCCAGCAGTCTC
CATCAAAGTCCAGGGAATCTTTCAACTCACCCCATTGCTTTCAGGAAGGACTTTTAACCATCAGACACAGCAGCAGG
CATGGTACTCAGGGCCCAGGATGCTTCTGGAGGGTCTTCCGTGCAAAGGTTTCATTCCCTCAAAAACCAAAGAAGGG
AAAGAAATCAATACAATTCAGCCTGGATTATTTTTGCCTTTATGCCAACACAGTTGTAAAATAGGGTTTCCCATATA
TTTTATGGAAGAAGGAGCCCCCAGAGTCAAATGGGCCTGGGGTCCCTGGAAGTGATCACATGGTCATGGGTGTGTGG
CAGCTAGGAATCCCTCCGGGGATTGTAGAGATACGTGTCTAAAAGGGGACAGCGAGAAAGTGAGTCTGTTCCAAACC
TGGGTTGTTCCCCTCCTCCCCTCTTCCCCCAAAAGGTGACCTGGATGAAGAAATAATCCCAGAGGAAGACATCATTT
CCAGAAGCCAGTTCCCCGAGAGCTGGCTGTGGACCATTGAGGAGTTTAAAGAACCAGACAAAAATGGGTAAGGCTGG
GATGACCCTGCTTCAACCCCCGCCGCCAGTACCCAGGGACAGCCCCCTCTCATCACACTAGAACTGGACAATGAATT
TGCAGGTACCTGGAGTCCCCCTTCTTTTCTTTCTTGGGGGAATCCCACAACCCAACCTAAAAAAATCAAGCCCTTGG
GCTATCAGCCACTGCCCCACACACTACAGTCCGTTCCTTTCGCATCTACTAAAAATTTATCTTGTGTTTGTTTATTC
TTCATTCATTATATTTTCTTTCTTTCTCACTGCCTGCGCTGTGACTCCTTTTCTCTCTACATTCTGTTTATCATCAT
CTTCCACACAACTCATTTCTTATCCTCACCACCACCACTCTCTGCTCCAAATTTTGAATTTTACACCCAGACTCCTC
TCTGCTATGTGAAGCGCCTACACCCCGTCACTAGTGTTACTCTCTTATCGCTGACCTCCCTTGTACCCTCCCATTTA
TTTCTTTTTTTTTTTTCTTTTGCCCTATCTACCTGCCTCTCTTTCCCATCCCATGTTTGCCATGTTGAATTATGTTT
ATTTAAGAATATGTTTAGAGAGTGATGTCTCTATTGATGATGACTACCTGCTGTCTCTCATCCGCGCGACATATTCA
TTATTTATACCATTTGGCGTACTTCACTTGTCTAACACAATCCTTATCCGTATATAAAGAGATGATGAAGAACCCCC
CGCCCGCCCCTGNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNTAACCCACTGAGCAAGACCAGGGATCCTTAACCCGCTTTGCA
CAGCAGGAACTCCTGGGCTTTTTTTTTTTTTTTTTTTTTTTGAGCCCTGAGATTTTTTAATCCCCCCCCCCTTTTTT
TGGCTTTTCTAGGGCCGCACCCGTGGCATATGGAGGTTCCCAGGCTAGGGGTCTAATGGGAGCTGTAGCCGCTGGCC
TACACCACAGCCACAGCCACAGCCACTCAGGATCCGAGCTGCATCTGCAACCTACACCACAGCTCATGGCAACACCA
GATCCTTAACCCACTGAGCAAGGCCAGGGATTGAATCTGCAACCTCATGCTTCCTAGTCAGCTTCGTTAACCACTGA
GCCAGGATGGGAACTCCCTTAAATTCCTGACATCTTCTCAACATCAACTCTCTTCTCAAGATCAACTCTCTCTCATC
TCATTTTTTTTTTTTTTTTTTTTTTTTTCTTTTCTAGGGACGCTCCCGTGGCATATGGAGGTTCCCAGGCTAGGGGT
CGAATCGGAGCTGTAGCCACCAGCCTACAGCAGTGTGGGATCTGAGCCGCATCTGCAACCTACACCACAGCTCAAGG
CAACACCAGATCCTTAAGCCACTGAGCAAGGCCAGGGATCGAACCCGAAACCTCATGGTTCCTAGTCGGATTCGTTA
ACCACTGTGCCACAACGGGAACTCCCAAAATAAGAGATTTTTAAAAACCGTTTTAGGATTCCAGAAACAACTGAGCA
AAAAAATATACCAATGGCTGAGTAATAGTCCATCATGTATCTGTACTACATCTTCTTTATCCACTCCTCTGGACACT
TAGGTTGCTTCCGTGTCTTGGCTATTGTCAGTAGCACTGCAGTGAACACCTGGTGCATTCAAATTATGGTTTTCTTC
AGTCTTTTCCATTTTTAATTCCTTTTTTTCCTTTCAAATAGAGAGCAAGGGGTCTAGCTTTCCTCAGGCAGCATAAG
CTAACCAATATTTAACACAATCATTCTATTTTCCTTGAGGACACTCTTATTTATAGCACAAGAACCTGGTTTCTCAC
CCATGTCCTAAATTAAATTTAAGTTTAGAAAAATTTATAAAAACAAATAGTAAGTAAGAAATGGTAAGGAGCACCAG
TGACTAATCAGACACCCCGAGGGTGATGAGTAAATGACAGTAGGTTGGGAAATAAGGATTTTGTTCAAGCCTCTGAT
TATAATTTTTTTTTTTGCTCTTGAAGAATAAGAACAATGCACAAATCTTAATAGATTTCTTAGTGTAACATTATTAA
TAATGTGTTAACAGTTTGTGCAGTTTCACTTGCATCAGCACTCTGCTTGCATTTGATCAGGTAATTTTTGTGTCATA
TATAACATTGTTTTCAGCATCATTTTTGATCAAGGTTGTTATCAAAATTCAACGGAGTAAATTTGAAGATGTAATTG
GCTTTATTAAACAATTCATGAATTGGGCAGCGTCTCATCTGGCAGGCAGAGAGATACTCAGAGGAGTTGTGAAAAAT
GGAAGGTTTTAATAGAATGAAGTCTAGGGCAAGAGAGTAATCGCAAGATACAAATTTCATCATTGGAGGAAAATAAC
AATTCAGGTGGGAGAGGATCTCCTTGGCTGAGCTACAGTATTTTCATTCGCTGGGCTTTTTACTGGGCAGGAAGAAA
GTCTTCCTTCCTCCTGCTGCAGTAAATTTCACTTCCTATTTGGGAGTGCAAGGTACTTCTCTTTCCTTTGGGGTCTG
TAATTGATGCTTCTTCCTGTTGGGATCTGTAATTGACATCTTCCTGTTTGGGGTAATTGACTTGCTTGGTGGAGCAT
TAGAGCTCCCTCTACAGGCCTTCCCTACTTCAATTTAGTTAAGGTTTACTTTTACTAATTTTTACAATGTAAATCAG
TGCTGTCCATTAGAAATATAATGCAGGTTGTAAACGTCATTTAAAATTTTCTGATAGCCCTGTAAAAAAGGGATAGG
TGAGTGAGTTCCCTTGTGGCACAGTGGGTTAGGGATCCTGCATCATCACTGCAGCAGCCCATCCCTGCTGTGGTGTG
GGTTTGATCCCTGGCCCAGGAACTTCCACATGCTGTAGGGGCAGCCAAAAAGAAGGGATGGTAGGTGAAATCAATTT
TAATAATACATTTTATTTAATCCAAATATATCCTAGGAGTTCTCATTGTGGCTCAGTGGGTTATGAACCCAACTTAG
TGTTGTGAGGATGTGGGCTGGATTCCTGGCCTTGCTCAGTGTGTTAAGGATCCGGCACTACCTCAAGCTTTGCATAG
GTCGCAGATGGGGCTGGAAGCTGGTGTTGCTGTGACTGTAGTGTAGGCTGGCAGTGACAGCTCAGATTCAGCCCCTA
GCCTGGGAACTTCCACATGCTGCAGGTGCAGCCCTAAAGAGAAAACAAACAAATATATCCAAAATATTATTATTTCA
ACATTTTGTAAAAACTTGCAAAACCACTATCACACTGATACTGTTACAATAATAAATCCATTAATATTTTAAAATAA
GCTATTAATAATCTCAAAATTGTGATATCTTTTAGTTTTATTTGTACTAAGCCTTCAAAATCTGCCATGTATTTTAT
ACTTACTGATATCTCAATTAGAATGTTAGCTTTTCATTAGAAATACTTTGATCTGTAATTACCATCCATAAAATTTA
CAGTTAAAAAGGAAAGTGTACCCAAGTTGTTGTAAATATTCTTTTTTCTTTCTTTTTTTTTGTATTTTTGACTTTTC
TAGGGCCACTTCTGCGGCATATGGAGATTCCCAGGCTAGGGGTCTAATTGGAGCTGTAGCCACCGGCCTACGCCAGA
GCCATGTCTGCAATCTACACCACAGCTCACAGCAATGCCAGATCCTTAACCCACTGAGCAAGGACAGGGATTGAACC
CGCAACCTCATGGTTCTTAGTCGGATTCGTTAACCACTGTGCCACAATGGGAACTCTGTAAATATTCTTTAAAAAGT
TATCCAGTCACTGAATCAAGCATCCTTTTAAAAATTGAGATACAGGAGTTCTCTGGTAGCCTAGCAGTTAAGGATCC
ATTGTGCCACTGCTGTGGCTCAGGTCGCTGCTGTGATATGGGTTCAATCCCTGGCCCAAGAACTTTCACATGCCATA
TGCACAGCCAAAAAAGTGTAAAATAAAACAAAATTGTGATCTAATTCACATACCACAAAAGTCACCCTTTGAAAGTG
TACAATTCAGCGGTTTTTAGTATATTCACGATGCACATTGTTTTTGTTTTTTGGTATTTTTTTTTTTAGGGCTGCAC
CCACGGCATATGGAGGCTCCCAGGCTAGGGGTTGAATCAGAGCTGCAGCTGCTGGCCTATACCACAGCCACAGCAAC
ACCAGATCTGAGCCATGTCTGTGACCTACACTGCAGCTTGAGGAAATGCCACATCCTTAACCCACTAAGCAAGGCCA
GGGATCGAATCCATATCTTCATGGATACTAATTGCATTTGTAACCACTGAGCCGCAATGGGAACTCCTGCACAGTGT
TTTTTCTTTTCTTTTTTTTTTTTTTTTCTTGTCTTTTTGTCTTCTCTAGGGCCGCTCCTGCAGCCTATGGAGGTTCC
CAGGCTAGGGATCCAGTTGGAGCTATAGCCACTGGCCTACGCCACAGCCACAGCAACACCAGATCCGAGCTGCATCT
GTGACCTACACCACCGTTCATGGCAACACCGGATCCTTAACCCACTGAGCGAGGCCAGGGATTGAACCCGCAACCTC
ATGGTTCCTAGTCGGATTCGTTAACCACTGAGCCACGACGGGAACTCCTGGTTTTTAAGTTGAAATCTGAGTTAACT
AAAACGAAATAAAAGTAGGAATCCAGTTCTCAACTGAGCTAGCCACATTTCAAGTGCCCAGGGTCCACTTACAGTCA
TCATTTTGGAGAGCACAGATCAGAACCTTCAGTTATGCTTGCCTTCTTCCCTTCTGCATATTTACCTATGAATAACA
TTACAAAGAAAATGAGAATTTCTCTCACAGCAACTCCCATCCACCACCACCACCTGTAAGATATCACTATTAATGAT
GTGTCTCTGGGCTCTGCCAGGGCAGGCGGAGCTTGGGACAGCTCTTGTGGTCAGGGGTGAGCCCTGAGATATTGGCA
GGGTCAGGAACTTGGACCTGAACTTGGATCCAGCCCACCCTCCCTGCCCCCTACCACCGACGCTGTGTTCTGTTTCC
ACCTGGGCAGGGATCTGCGTGGCTGACCCCTATGAGGTTGTGGTGAAGCAAGATTTCTTCATCGATCTGCGTCTCCC
CTACTCCGTTGTGCGCAATGAGCAGGTGGAGATCCGAGCTATCCTCTATAACTACAGGGAGGCAGAGGATCTCAAGG
TGAGCCTCTAGTGTGACAGGCATGATGGGGAGCTTGGAGGGAGGGTCCATGGCACACTCTCCTGACTTGATACTCCC
TCTTCCTGGCAGGTCAGGGTGGAACTGCTCTACAATCCAGCTTTCTGCAGCCTGGCCACCGCCAAGAAGCGCCACCA
ACAGACTCTAACGGTCCCAGCCAAGTCCTCAGTGCCCGTGCCTTACATCATTGTGCCCTTGAAGACTGGCCTCCAGG
AGGTGGAGGTCAAGGCCGCCGTCTACAACCACTTCATCAGTGATGGTGTCAAGAAGACCCTGAAGGTCGTGGTGAGT
CTTTGGGGATACCTGCTGCCCCTTGTCCTTCAGGAAAGACTCCTGTCTTCCTGTGCTGTGAACCCAGGTTGGAGACC
CAGGCTAAGAATACGGAGTACTTCTCAGAAAATTTAGGAGTTCCGGAAGTTTGGAAGCAGGGCTGGGATTAGGGTGA
GGCAAGTGAGGCATTCTCCTTGGGCATGGAATTTCAGGGGACACTCCAAAGCTTAGTAACAGAGATCAATGATATTT
TTTCGTTAAAATATAGTTTAATGTCAAATATGACATTTCGTAACACATTTCAGCAGAGGAGTTTTCTCTTGACTAAA
AATCTTGGGAGGAGTTCCCATTGTGGCTCAGTGGTTAACGAATCCGACTTGGAACCATGAGGTTTTGGGTTCGGTCC
CTGGCCTCGCTCAGTGGGTTAAGGATCCAGCGTTGCCATGAGCTGTGGTGTAGGTCGCAGACACCGCTCGCATCCCA
CATTGCTATGGCTCTGGTGTAGGCCAGCGACTGTGGCTCCAATTAGACCCCTAGCCTGGGAACCTCCATGTGCCGAG
GGAGCGGCCCTAGAAAAAGGCAAAAAAAAAAAAAAAAAAAATCTTGGGAAAGCATATTTCACAGAACAAATATTATA
AAGCCATAACATACAATGCTAGAACAGAGGAAACGTCTATTTCTACCTATGATTCTTACCTTAAAATATGCATTAAC
AGTTACTTTTCCATGTCCTATGATTAAACATATAATAGATAAAATCAACAATAAAAATAAAAGTATTATCATCTTTT
AGTAACGTTTTAAAGCAAAATGTGAGATCATAAACAAGATCAAAAATATTTAATTCAAGAGTACCTGTTGTGGCTTA
GCGGTAACAAAAATATTTAATTCAAGAGTTCCTGTTGTGGCTCTGACTAGAATCCATGAGGATGTGGGCTTGATCCC
TGACCCTGCTCAGTGGGTTAAGGATCTGGCATTGCCATGAGCTGTGGTGTAGGTCATAGAAGCAGCTTGGATCTGGC
ATTACTGTGGTTATGGTGTAGCCAGCAGCTGCTGCTCCAATTCAACTCCTACCCTGGGAACTTCCATGTGCTGTAAG
TGCAGCCCTAAAAAGACAAAAAAAGTAATGCAATATATTAAGAAATCAAAATTAATGCCCCAAACCCTCACAACAAA
CAAAATATCAAAATTTTAAATAGAGACAGGATCTGACAGTGTCAAGGCAAACCATATTGGAGCCTGAAGCAGAAGAA
AAATGAGTTGCTCCATAAATGTGCCTGTATGTATTTTTAAATGGTTAATTTTCCCCAAAAACATTACAGTAGCTGAA
AAAATATTGAAACATTGAAAACCAAGTGTATTAAAATTGACAGAGTGATTTTCCATTGAAGTATTTTGTTTATACCC
AAACCAGAATTTATTATAATTTTTCTTTATTGGCTTTAATAAAAGCAAACTCATATTTTTTTCAACTACTTTACTGT
TCTGGAATAAAATTAACCATTAAAAATATGTGAAAGTATATATTTTGGGGCACATATTTTTCTTTCTTTTCTTTCTT
TTTTGGGGGGTGTCTTTTTAGGGCCGCACCATCAGCATATGGAGGTTCCCAGGCTGGGGGTCGAATTGGAGCCATTG
GCCTATGTCACAGCCACAGCAACGCCATTTCTGAGCCAAGTTTGTGACCTACACCACAGCTCATGGCAATGCCAGAT
CCTTAACCCACTGAGTGAGGTCAGGGATAGAACCTGCATCCTCATGGATACTGGTCAGATTGGTTTTCACTGAGCCA
CGATGGGAACTCCACACACATTTGTCCTTTTGCCTTGAGTTTCTATATGGCTCAGCTTGGGCACTGGTGAGAAGAAA
GCCAGGATTTTGTTAGAGTTTATATTGCCCAGCTCCCAAAAGCCAGTGTGCCCATCACTTCACAATTCTGTACTCAC
TGTGGCTGGTAGCTTGAAAATCACCATGTTGGGAATATTTACACCAAGGAAATTGGCAGCACTACAAATTAGGAACT
TTTCTTCCTGAAAAGCTGGATGTTATATATTTACCAACACACCATTGGAGGCATCTTAGTCTGCAAAGGAAAATCTG
GGAATTACTACCAGGTGAAAGGAGAATGAGTTCTAGGAAGACAAAAACAGCCACCGTCCACCATGGAGATTTATGTG
TAGACACATAAGGGCTTGTAGTGGGCCTTTGATCCTAATTAAGACAGTTCTGATTTTAACTGAGCCCTTACTATGTG
CTAGGCACTATGTTAAATACTTGTGTGAATCCTTTCATTTCTTTTGTGAGAGGGGGGTCTTTTTAGGACCACACCTG
TAGCATGTGGGAGTTCCCAGGCTAGAAGCTGAACGGGAGCTTCAGCTGCCAGCCTTCGCCTCTGCCACAGCAACGCC
AGATCCGAACCACATCTGCAACGCCACACCACAGCCCATAGCAATGCCGTATCTTTAACCCACTGAGCAGGGCCAGG
GATCAAACTCGGGTCCTCATGGATACTAGTCAGGTTCATTACCCTGAGTCACAACAGGAACTCCTCATTTCTTTTTT
CTTTACTATTTATTCTCATTTGTTTATTTGAAAATGTTGTTTTACTTTTAAATTATTTGTTTTATTTTACAATTTTT
ATTTTTATTTTAGTTAGCCTATTGAGAGGCACTGGGTTAAAAACAGACTCTGGAACCAGACTCTCAGGTTCAAATCC
ACACTGTGTTCTACTAGCTATGTGACCTTGGGCAAATGACTTCATCCATCTGTACCCCAGTTCCCCCATCTTGAAAA
TGGAAGTGATAATAGCAGTATCCACCCCATTGAGTCGTTGTGAGGATTAAATGAATTAACCCCAGTAAAGAAATCTT
TTAGGCACATAGGAAGATTTCTATAGATTTTGTTAGGTCATTATTAACTTATAATTTTATTATTAATCTATACAACA
ATGGGTACGAGGTAGATGTTTATATTATGTCTTTATAAGGAAGAGAGCTGAGGCACAGACAGGTGAAGTAAGTGACT
TCCAGTCACACAGCTAAGATCTAGTGGATGCCATCGTGCATATGCTACAGTAATCCCCAGAACAATGCCTCGCTGAC
CAGCTGTCTGTCTGTCTGTCCTTTTCTTCACGGGACTCCCCCTGCCCCCAACACTATCCAGCCAGAAGGAATGAGAG
TCAACAAAACTGTGGTCACTCGCACACTGGATCCAGAACATAAGGGCCAACGTGAGTCAGCCACAGAAGGGGTGAGG
GCTGGGTGGTTGAGGCAGGGTAGGGTGGGAGGGGGGTGGTTGAGGCAGGGTAAGAGTGGGAGGGGGCTGGTGCAATG
GGTGTCTCCCATTCTCCCGGCAGAGGGAGTGCAACGAGAGGAAATCCCACCTGCGGATCTCAGCGACCAAGTCCCAG
ACACGGAGTCAGAGACCAAGATCCTCCTGCAAGGTGAGAGGCCCTTGGCTTCGACCCCAGGGGACCCAGAACTGTGT
TGGGGGGGCATGAGCCCAGTTCCATCTCATCCCTCCTCCTCTTCAGCTAGAATTTCTCTTTGATCTGCTTCAGGAAG
GCTCCAGGCACTATTTAGTTCAGCCAATAGCTTTTGCTGATGAAGAAATTTATTATTTTTTAATGAATTTATTATAT
TTATAGTTGTACGACGACCACCACAACCCAATTTTATAGGCTTTCCATTCCTAACCCCCAGCACATCCCCTCTCCTC
CCACCCTGCCTCATTTGGAAACCATACGTTTTTCAAAGTCTGTGAGTCAGTATCTGTTCTGCAAAGAAGATAGATCA
TTGTAGCTCTGATAAAGAAATTTAAATAAGAAGCAGTATAGTTCCAGAGCAGAAATTCTGGATCTGATTGCCCTGGA
TGGGGAACTCGGGCAAGAAGGGACAAGATAGATCTGAAAAGGCACCTTGCAACCTGTAAGGTGTAAAGTTTTGGGAG
GAGACCCTTGGTTCCCTCATCTGTGACGGGGGCAAATAACAGTATGGTTACCTAAGGGTTGTTGGGTGGGATTAAAT
GAGATACTATACAGTGTTCTCTTAGAATAGAGCCTAGCAAATAGCATTAAGCACGATATAAATATTCCTGACTATTG
TTACTGGAATTATGTTACCACTGGTGTGTAACGAGAGGAACCAGGGACTGGAAATCCCCTGTGAAGCACAAGCTCAC
CCCCACCACTCCGCAAATGCAGAATCCCCCTCCAGCTGCTCAGCTCCTCCCATCACATACCCTCCAGCTGTCCCTGA
CTCCTTTGGCCCTGGCTGGTCAGAGTCTGGAAATGCTGGGGGCAGCCCTGGTCTTGAATGCCATCTTACCGTCTGGC
TGCAGGGACCCCGGTGGCCCAGATGGTAGAGGATGCCATCGACGGGGACCGGCTGAAGCACCTCATCCAAACCCCCT
CCGGCTGTGGGGAGCAGAACATGATCGGCATGACGCCCACAGTCATCGCTGTGCACTACCTGGACAGCACCGAACAA
TGGGAGAAGTTCGGCCTGGAGAAGAGGCAGGAAGCCTTGGAGCTCATCAAGAAGGGTATATGCCGCACCTCCTCCTC
TGAGCTGTCTAGGCCCCTGAGACCCCGCCCCTCCGAGCCCCCTCCAACCAGAGGCCCCTCCCCTCTAGAGGCCCCAC
CTCTCTGAGCCCTCTCCAACCAGAGACTCCGCCCCTCTATAGGCACCACCCCTCTGAGCCCCTCCCAACCAGGGGCC
CCGCCCCTCCTCTGAGACCACCCCCTTGCTCCTCTCNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNCCTCTATCTGATCCTCCC
ACTTTCTACTTTAAGCTCCCCTTCCCCACCCCAAACTTGTCCCCTGCTCAGAACCCTCTCTTTCTTCTCTGTACCCC
TGTCCCACCTCTCACAGAATCTTTATCCTCTTTCTAAGCCCCTCCCCTCCCTGGCCTACCCATGGTAGCCACCCCCT
CCACTCAGCCTCTGTTGACACTTCTCCCTTCTCGGCAGGGTACACCCAGCAACTGGCCTTCAGACAAAAGAACTCAG
CCTTTGCCGCCTTCCAGGACCGGCTGTCCAGCACCTGGTGAGTCTCCAAGATCTGCTTGCCCATCCTTAGCCTTGCA
CCTCCCTGAGCAGGGCCTGGATCCCGGCCTCAGGTGGTCTAGGTTGGCCTCGCCCACACAGCCCTGTGCGACTTGAC
CCCTCTACTCACGAAGTCAAAACACCAGCCAGATGAGTGGCCTGCATGCCACACCGGGTCCTGAGTTTGGGGAAGAG
AAACTGGGCGGACCAGGCCAGGCCCCGCCTCTCTCTGTTCATTGCTTGGCTGGGATGCAGTCTTCGGATCCCAGAGC
CAATTGGCTCATGCTCTGTGTCCGCAGGCTGACAGCCTATGTGGTCAAGGTCTTCGCTATGGCAGCCAACCTCATCG
CCATCGACTCCCAGGTCCTCTGTGGGGCCGTCAAATGGCTGATCCTGGAGAAGCAGAAGCCTGATGGAGTCTTCGAG
GAGAATGGGCCCGTGATACACCAAGAAATGATTGTAAGAGGAAGGGACTCAGAGCAGGCAGGGGGAGAGGGGCATCT
GAGCATCACAGGTTAGCGGGGTGGGGGGGTGGGAGGAAGACTCCACCATCCACCCATGGCCCAATCCATTGTGCCAG
GGGACAGGGGATAAGGGAGCTGGGAGTGCCACTCCTCCATTGCAAAAAACAAAGACTTGCAGGATCCGGTGCAAAAG
GAAAGTTCCCAGGTCACAGAGCTGCTTAGAGCCGTGGTCCTCAAAGTGTGGTCCCCAAGCCAGCAGCATCAGCACCA
CCTGCAAACTTGTTAAAAATACACATTTTCAGGATGGACTCCAGAGGCACTGAATCAGAAACAATAGGGGCAACGTC
TAGAAATTGGAGCTTTAACGCACATATACACACATCTCTGCTGATGCTGGTGTGTGCTGAAGTTGGAGAGTTGCTGC
CTTAGCCTGACCTTGCTGGCTTTCACACAGCTTTCTCCTGCCCCCCTTCACACTCTACCTGGACTGCTAGAAGCCTT
GCTCTGTCCAGCCACAGGGCCTTTGAACATGCTGTTTCTGCTGCCTGCCCTGCTAACCCCTGCCCTCTTTGAGAGTT
GACTCCTACTCACTCTTCAGATTGTGGTTCCATCTGTCACCCCTCAGAGACACTTTTCCACGACTGAGTCACTCTTC
CACTGTCCATTCTCAATGCCATCTCCACTTCTCCTGCACAGCACTCATCAGTTTGTAATTATATATCTGTGGATGAC
CTGGTTGGCTCATGTCTGTCTCCCCTACTAGACAGGGAGCTCCATGAGGGCTGGGCTGGGGTCTGGTTTTCTCCCAC
CATCTTATCCACAGCTCCATCAACATTTGCAGAATGAATGAATGGATACTAAAGAGCTTGGCCCTCTTGGGGAGACC
CTGGGGAGAGACCCAGCCCTGCCTTGACCTGCTGATCCTACAGGGGGGTGGTGGGCATGTGGGGACATGATGTTCAC
CCGCTCCGGGCTTCCTGCTTCCCCTCTAGGGTGGCTTCAAGAACACTGAGGAGAAAGACGTGTCCCTGACAGCCTTT
GTTCTCATCGCGCTGCAGGAGGCTAAAGACATCTGTGAACCACAGGTCAATGTAAGTGTCCCTTGCCTCTCCCTCCT
CCCCTCCCCTGCTCAGGACACATCAGGTGAGGTATGGATTTGGGGCCATTTCCAGTCCTCCCAGTGTGACAACCACC
ATCACAGTGGCCATAAGAGTACCTAACATTTATCGAGCCATTAACTAAGATACTCACCTAAAAGCTTCACATGTTTA
AGTCCTGTAATCCTTGTAGCAGCCCAAGAGACAGGCTACCCTTATTATCCCCAGTTTTTAGAAGAGAAAACTGGAGC
TCCCATCATGGCTCAGCATAAATGAATCTGACTAGTAGCCATGGGGACACAGGTCTGATCCCTGGCCTTGCTCAGTG
GGTTAAGGATCTGGCGTTGCTGTGAGCTGTGGTGTAGATCACAGACGAGGCTCGGATCCTGTGTTGCTGTGGTATAG
GCTGGCAACTATAGCTCCTATTTAACCCCTAGCCTGGGAACCTCCATATGCTGTAGGTGCAGCCCTAAAAAGACAAA
AAAAAAAAAAAAAAAAAAAAGAGAGAGAGAGAGAGAGAAAATTGAGGCACAGAGAGATCAAAGATCAGGTCCTTTCC
GCCTGTTCTCCCATTTCTAGAGAGTCATAGCCAATTTCAGCAGAAGTCCTCTCAGTTTGCTTTCCACAGCACTCCTC
CACATGCCTCCTTGCTGCTTCCCTAGAGAAAACTCAAGACACAGAGCTTAAAAAGAGGAGAAAAAAAATCCTCAAGA
CCATTTCCTTAGTTTAGAGGGTCTTTCAGGGTATTTTTTTAAAGGAGTCCATGATCCCAAAAGGGAAGGGATTTAAA
ATGTTGACTATTCACTGTCCCCTTTTCCTCTGGCTTTGGTTCTGAAGCAGAGAAGTTTGAAAAGACAGGCTCTGGAG
AATCTGTAATCACTCCATCTGCTTTGCCCTGGGATTTTGAGGCTGGGTTGCTTGACTTTAGCTTCCCTACAGGGGAA
CCTCAGGCTCTCATCTTCAGCCAGCTGCTTCTACCTCCTCAGAACCCCAGAAAAGGGATGGAGGGGAGGGGCCGTTG
CCTTTAATGCCCAAAAGGGCCCAGGCCTTCCTGGTTCCAACCTGGAAGATTTGAGAGAAATTATAGTAGAAATGAGA
CAACACTAGGACTAGGCACGGGGTAGGGGTGGGGATGTCAGAGAGAAGTGACTTCAAAGCCTGACTCTCAGGCACTT
CCCCTTCAAGGCCTTAATGTGTGCATCTGTAAAACGGGTATGGTGGTCTTTGTATTGTTTAGGACTCTCTGCATTGT
CCTAGATGGAACACAAGTGTGACCCAGATTATGCAAAAATAGGGTATTTATTTTAGGGATCCAAGAATTTATCAAGT
GCAACGATAAAAGAGTCCTCAGGGACTCTGCCAGAATGCTTCGTTTTTCACGTCCTCCCATATCTTTCCTTCCCTTC
TTGCCTAATAATTCAACTTTCCTGGCCATCCGGCCTGCCTGGCCAAACTGTCTTCCTTGGGGAAATAGACCAAAGCA
CCAGCAGCAGAATCTCAGTGACAGATTCTGATTGGCTCACCGTGGGTCAGGTGATCACCTGTGGACCAATCAGCTGA
GGGAGGCAGTAGGTCTTAGTGGGCAACTATGTGCGCTTCTGGTGCGGCCTTGTGAGTGGAAGTGAGGTGTTCTAACA
ACAGTCATCGACAGGTGTAGAAGAGATTCCTGGGCAGGCAAAAGGATCATTTCTACTGTAATATAACATTTTTTACT
ATACATATTATAATGAAGTATGGCATAGGCTGTGGAACCCGACTGCTGGCATTTAAATCAGGAGTATGCTGAACCCA
TCCGTGTAAAATCTGTAAAACCAGTTGTTAAATTTCCAGGAATTTGCAAGCTGGCTGTTAAACACGATCGTGATTAA
ATTAAATTATAAACTTACAGTGAAAAACTGTAAACATTAAACAGTAAAAACAGGCGTTCCCGTCGTGACGTAGCGGA
AACTAATCTGACTAGGAACCATGAGGTTTCGGGTTCGATCCCTGGCCTGGCTCAGTGGGTTAAGAATCCAGCGTTGC
CATGAGCTGTGGTGTAGGTCGCAGATGCGGCTCAGATCTGGCGGTGCTGTGGCTGTGGTGTAGACCGGCAGCTGTAG
CTCCAATTAGACCCCTGGCCTGGGAACCTCCATAAGCCTCAGGTGCAGCCCTAAAAAGACAAAAAAGATTTTTAAAA
AAAGGACAAAAAAAGGAGTTCCGTGGTGGCGCAGTGGTTAACGAATCCGACTAGGAACCATGAGGTTGCGGGTTCGG
TCCCTGCCCTTGCTCAGTGGGTTAACGATTCGGCTTGCCGTGAGCTGTGGTGTAGGTTGCAGACGCGGCTCGGATCC
CGCGTTGCTGTGGCTCTGGCGTAGGCTGGTGGCTACAGCTCCGATTAGACCCCTAGCCTGGGAACCTCCATATGCCG
CGGGAGCCGCCCAAGAAATAGCAACACCACCAAAAGCCAAAAGCCAAAAAAAAAAAAAAAAAAAAAAAAAAAAGACA
AAAAAAAAAGTAAAAACGCAGGTAGTAAACACTTAAAATGTATCACTTCCTAAACATTTTGCTATCTTTTATCATGG
TTCTTTTGAGAATTTATGTGTATTGTACTTGTATAGTGGAAATATTATGTAATGTTGAACTACTGCCCATCTCTTCC
CAAATCTACATTCAATGATGTGGGTTGATTGATGGATTGAAAGCAGCCATGATAATATTGACATCATAGAAATGACA
AACCCTTCAAATTATGTTTTCCCCCAACCCCTATCTTTCTGGGTCACAGCATTTTTCTCTGACAGGAGGATAATGAT
GAAAATAATACCTACCTCATAGTATATTATGAGATTAAGTGAGCAAGTATATGCCTGGGACATAGTAAGAGCTAGCT
ATGATGGGGATTACTCTCAGATAAGAAGTGTTCCCTTGGTGAGCTGAATCTGGCTCACACTAGCTCACGAGTGCCTA
CGGGGGGCATCTCTACCCCACTCCATGTTCAGGGACTTCACATTGGTAGCTTAAAACTGACCATGGTAGAATTTTTA
CACCACAGTAATTGGTGATGCATAAAGGAGCACCCCTCCCCCAACCCCATGCCTCCATTGGAGAGCTGATTGTTAAA
CATTCACCAGCACACCATGGGGTATACAGACTGCCCCCCCCATCCCCGCTGCCAGCACATAGTAGGTACTCAGCAAC
AAAGCAGCTCACAATGAGAAAACTTCAAAAGTAGGTAGTAGATCCAAGGCAGGTCCCAAGGACAGATACCATCCTGG
CGCCCAGGAAGTGATGCTTGTGTGATCCTTACTAGTTCTCTGTGGCAGCAACGCCCACTTGATCAGAATACCCAATC
CTCTTTCTCATAGAGCCTGTTGCGCAGCATCAATAAGGCAAGAGACTTCCTCGCAGACTACTACCTAGAATTAAAAA
GACCATATACTGTGGCCATTGCTGGTTATGCCCTGGCTCTATCTGACAAGCTGGATGAGCCCTTCCTCAACAAACTT
CTGAGCACAGCCAAAGGTAAGAGGCAGCCTGGAGAGATAAAGAAGGGGGTGCATGGCTAGGGTTTGAGGGTGGTCCT
CTCAAGCTGGGATGCATGCCTCTAAGCTGCACTGGGATGTGCATCTCCAAGTGGAGCTGGGCTGGATGGCTCTACAA
GGTGAAAAGCTCTCATTGTAAACCACACAGGAAGGCTCACTGCATAATTCATGACAGCAGTGAGGTGTCATTAAGAA
CATGGGCTCTGACCTCAGGCAGACTGAAACCGAAACCCCACTCAGCCACTTTCTCACTGCCTGACCTTGGACAAGTC
ATTTAACTTCTCTGGACCTTAGTTTCCTCATCTTAATACCTACATCGCAGGGTGGTCATGAAGATTAAATGTATAAT
GCAAGTAGAAGAGAGTCTAGCACACAGTAAGAGCTCTGTCACTGATACCATTAGTGCCTTTAATTTTATTTTAATTT
TTGTCTTTTTAGGGCCACACCTGCCGCATATGGAAGTTCCCAGGCTAGGGGTTAAATTGGAGCCACTGCTGCTGGCC
TATGCCACAGCACAGCAATGCAGGATCCGAGCCATATATGCAACCTAGCTCACGGAAATGCCAGATCCTTAACCCAC
TGAGCAAGGCTAGGGATTGAACCCGCATCCTCATGGATCCTAGTCAGATTCATTAACTGCTGAGCCACAAAGGGAAT
CCACCTTCAATATTGTTAAAAATATTATCATTATCTGAAAGCATAGGGAACTTAGCACAGTGCCTAGCACAGAGTGA
GTGCTTAATTTTTGGTCCCAGCTGATGACACTGTATCATGTTTGCACTCACTGATGTGACATATCTCAAGTAATGGA
ATGTAACATATACAAAAGTCATTTAACACAAGAATAATTTATTGGTGGTGGCCGGCTCTCCTCCACACAGAGATGCA
GAGATCTAGGCCTCTATCTTTTCATAGCTCTGCCGCTCAGAATCCATCCATGTAAGCTGAGGGGGAAATAGTCAGGA
AGACTGTGCAAGGGAGGTGGACCAAACATGGAAGGGGTCCCATCATTGCTGTGCACATTCCATTGGTCAAAGCTTAG
TTATGTGGCCATACCTACCTGCAAAGGCATCTGGGAGATGTAGTCCAACTCTGTGCCCAGGAAGAGGAGGGTATGAT
TCTTAGTGACAGCCTCTGCCATCAGTATTTTCTTAGGCACTTGTGACATACAGTGAATACAGTGCAGCCCTTCCCAT
TATGGCCTCACACCTCAGTTGAGGAGGGAAAATGAATTAATAGATTACTGTAGAACATTATAGCATTGGGATAGTAG
AAGCACAGGATGCTTTAACGGACAGGAGGAAGAAGGGCCTCACTTCCTCTTAGGGTGCCATTGAAGCTGAATTGTGC
GGGGTGAGAATTAACCACAGGTAGATGGAGAAAAATTGCTCCAAGTAGAGGGAACAGAATATGCAAAGGCTCATAGG
TTTAAAAAAAAAAAGAGCAAGTTTAGGGAATCTCCTGCAGTGGGGCTGCAGTTGAGAATTCAAATGGAGGAGTGAGG
GTTGATGAGGGAAGAGAGCAAGGCAGAAGACAGCAGATTGAGGGTCTTGAATGTGGGCCAGGACACTTGAAAACCAA
GTCCAGTATGAGTCTTTTTTTTTTTTTCTGAGCTTTCTCTGAGCTATTTACAGGCTGAACAGAGCATTGAGAGTGGG
GGTTCTCTCTGCAGAAAGGAACCGCTGGGAGGAACCTGGCCAGAAGCTCTACAATGTGGAGGCCACATCCTACGCCC
TCTTGGCTCTGCTGGTAGTCAAAGACTTTGACTCTGTCCCTCCTATTGTGCGCTGGCTCAATGAGCAGAGATACTAC
GGAGGTGGCTATGGATCTACCCAGGCAAGTAGCCCCACCCCCACCCCACCTCCACCCCAGGCACCTGCATCCCAACC
TCTTCTGGCCTCCCACTAGCCTTCTGGAGTAGGCACTGAGACCAAGAGAGGTAGGTCTTCTGTCCCATAAGCCAGGA
TGGTTGGAATGAAGTTGAGAAATCTTTTTTTCCCCCCTTATAAACCCATCTCTGGATCTAGACTACATTCTGAGTGC
TCCAAGCTGTGTTCTGAGCCTCTCTTTCCCTCTTGACATCTAGGTCATGTTCTCAGGGCTCAGGTTCAGATGTGAGC
CTCTCTCTCCCCCTGGTTCCCCAGTTCCACCAGATTCCCTATCTTATCCTGTCTCACTGGTAGGTTCTAGATCCTGT
TCATCTCACCAGACCCCCAATATTACCTTGTCTCATTGGTAGGTTCTAGACTGGATTTTTAGTTGTTCTGGGCCATT
ATCCAAGCTTCTTTCTCTCACTTGTGGGATCTAGACCATGTTCTCAGCTCCTTCAGGCTCTCAATATTACCCTGTCT
TACTGTGAGTTCTAGAAAAGGGTCTCAGCTATTCTAGCCCCCAGTAGGTTCTAGACCATGGGTTCTTTAGCCCCCTT
TATTTCTAGTGGGCTCTCAATCACATTCTCAGTGTTTGGGATTCCAAATCAGATGCTCAGTGTTCCCAACTTTACTC
TTTTTTAATGAGTGGGTTCTAGACATATTCCCAGCACTTCTAGACTCTTGTCTTAGATGCTCTCCTCTAGATGGGTC
TAGACTACTTTCTCACTGTGGCTAGACTTTCAGTCTTATGTCTGCCCTTTCTGGTGAATTCTAGACATGTTCCCCAT
GTCTCCAAGCTCTTGTCTGAACCCCTCTCACTCAGAGAGTTCTAGAACATGTCCTCAGTAGCCAACAACCCTCGATC
TTGTTCTTGAAGGCCACAATGGGTGGGTTCAAGGCCACAGTTTCAGGGCCCCAGCTCTGATCTGAGACTCTTCATCC
CTCAGTGGGGTCTAACAACTTTCTTGTTGCCCAGATTCANNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNAAGGTAGCTGCGGGA
AACTTTCCCAGGGAAACGGTATTCCGGTGTGAAATGGTATGGACAAGAAAAGCTATTTCTGTGTGAAATTGTTATCC
GCAATCCAGGCTCTGGACCCCTTCCATGAATTTTCTGCAGTCCTCATAGTAGTGCTTCGAGGTAGGGTGACCAAGCT
ATTCTGCCATTCCTGAGACTCTCTCAGTGTTCGCACTCCAAGTACTGCATCCTGGGAAAAACCCCTTCCCCCAAGAC
GGGACCTGGGACCCTTGGCTGCGGGGCTTGCACCTGGGAAATGTCTCCTTGAGCAACAACATACAAAGAAACCAAAT
GGGACTAAAAATAGCTGCATGGGCGTTCCCGTCGTGGCGAAGTGTTTAACGAATCCAACTAGGAACCATGAGGTTGT
GGGTTCGGTCCCTGCCCTTGCTCAGTGGGTTAACGATCCGGCGTTGCCATGAGCTGTGGTGTAGGTTGCAGATGCAG
CTCGGATCCTGCATTGCTGTGGCTCTGGCGTGGGCCGGTGGCTGCAGCTCTGATTCGACCCCTAGCCTGGGAACCTC
CATATGCGGCGGGAGCGGCCCAAGAAATGGAAAAAAGACAAAAAAAAAAAAAATAGCAGCATGCTTGCACAGTTGGG
GCAGATTATGGACAGCAAGATATAAAAAGACCAAAAACCCAGCTGCCATATCTGAGGAGCCAGGAGCAAAAGCTGGG
TGCTGTGCATGCCCTCTGCACACAGCCCCACCAAGGGGGCAGGCAGACCACCTAAGCCACCCCTCTGGCACCCCTAC
CCTCACCCCACTTAAGGAACCAGCTACACACACACACACACACACACACACACACACACACACACACACCTGCCCCA
AGTAAGGGACACACACGCACATCTGCCCCCAGCAAGGGAATACTTGTTTTCCTTTCTTCCTGCTGCAGCAGGAGCTA
AATAAAGCCTTGCCTGAATTTCTTATCGGGCCTCTTACTCAATTTCTGTTGACTGGGAAAGCCAAGAAGCCTCATGG
TTAACACCCCCAGTCTGGGGCAAGCCGGAATGGTCAGTCACTCTACTTCAAGGTAGACATTAGGACTCCCTTTTCCA
GATGCAGAAAAGAGTGCCCAAGAGAGGTTGCCTAACTGTTCCAGGTCAGCCCCCAAGTCAGAACACAGGAGGAGAGC
CAAGCAGACCAGACCACGCTGGGAAGGAGTTCAGGAGATTTGCTCATCATTCTGGCTGTACCCCTCATGGGCTACCA
GCTTTGACCCAGCTGCAGCGGAGCCTATAAGAACCAGTGAATTTGTGATTCTCAGAGGAGGAAAGGGGGAGGGGGAA
AGGACAGAAGAAGAGGGAGGGGAGGAGGAGGGAGAAGGGGAGGAGGAAGAGATGGGGGGAGAGGAAAAGGAAGAGGG
GGAGGGAAGGGAGGCGCAGGGGAGGAGGATGGGGAAGGAGGAGAGGGGAGAAGGCTAACATATTACACTTATGATGT
TCCAAGTATCTACTAAGCACTGCCTATATCTTACCTCGTTTAATCCTCATCAAACCCCTATGGGATTAACTCCTCTT
ACTCTCATTTCCATGGAACCAAAGTCATGGGGCATGGATTGGAACAGCCGAGGTCCCCATGTCAATGAACCCTGGAA
CCAAGATTTGAACCTAGGCAGTGCGACTCCAGAGCCTATCTCATAACAACTCCCCATGGAGTTGAATCCTCAGAACT
TAATCCCATCAGGTAGGCAGGGGTTCATCACCCTACCGGATAATCAGGTGACAAAACCAAGAGATGAAGGCATGTCC
CCAAGGTCTAATTGCCTTCAAGCTGGGGAAGTCTCTTACCAAAATCTGACCACGATCGCCATGGCCACTCACCTGCA
AGCAAAGAGAAGTCTACAGATCCCTTTGATTTTTCTTTCCTCTCTTTTATGGCTGCACCCGCAGCCCATGGAAGCTC
CCGGGCTAGGGGTCAAATCTGAGCAGCAGCTTCCAGCCTACAGCACAGCCATAGCAAAACAGGATCTGAGCCACATC
TGTAATCTGCGCCACAGATCCTTAACCCACTGAAGGAGGCCAGGGATTGAACCTGCATTCTCATGGACACTATGTCA
TGTTCGTAACTCACTGAGCCACAATGGGAAGTCCCTATAGATCCCTCTGAGATCTGGCCATAAGCCATCCTTTCACA
ACCAGGTACCCTGTCTCCCTGGGTACCAGTGATCACAGTGGTGAGTTATGAAAGTGGGAACGGGATGTGAAGAGGAA
AACCCAGTCTCTTTCTGGGGATTTACCTCTATCAGCTCACGAGTTCTTCACACTTTGCCAGGTAAGAAAGGATGGGA
TACCAATGTTCATTGCCGCCCTACACACAGTAGCCAAGACGTGGAAGCAACCTATGCATCCATATGCAGAGGAATGG
ATAAAGAAGATGTGGTATATACATACAGTGGAATATTATTCAGCCATAAAAAAGAAGGAAATCATGCCATCTACAGC
AACATGGATGGACCTAGAGATTATCATACTAAGTGAAGTAAGTCATACAAATTTACAGTTAACCAAGGGGATAGCAG
GGGGTGGGGAAAGATAAATTAGGATTTGGGGATTAGCAGATACCCACTGCCATATACACAAGGACCTACTATATAGC
ATGGGGAGCTATATTCAATATCTTGTAATAACTTATAATGGAAAATAATCTAAAAGTAAACATGTATGTGTGTGTGT
GTGTTCACTTTGCTATACACCAGAAACTAAAACACCATTGTAAATCAGCTATAATTTTTTTTAAGGGTTTGGGAGTT
CCCTGGTGGTCTAGTGGTAAGGACTCAGCACTTTCTCCATTGCTGCCCAGGTTCAATCCCTGATCTAGGAACCGAAA
TCCCACATCAAGCTGCTGCACACCACAGCCAAAAAAATGAAAAAAAAAAATTTTTTTTGTCTTTTTGCTATTTCTTG
GGCTGCTCCAGCAGCATATGGAGGTTACCAGGCTAGGGGTCAAATCAGAGCTGTAGCCACCGGCCTATGCCAGAGCC
ACAGCAACACAAGATCCGAGCCGCGTCTGCAGCCTACACCACAGCTCACGGCAACGCTGGGTCGTTAACCCACTGGG
CAAGGGCAGGGATCGAACCCACAACCTCATGGTTCCTAGTCGGATTCGTTAACCACTGCGCCACGACGGGAACTCCA
AAAATGAAAATTTTTTTAAAATTTTTAATGGTTAAAAGAGGGGGGGAATATCAGCCACTCTTGGCCCCACCCGCATC
CACCTTGCCAGGTTAGCATCCTATCCCCCGCTGTCTCACTAGCCTTGAAGCACTGCCTGACACATCCAGGCATGTAA
CAGCACAGCCTCCGAGCAGGTGAACCTCTGTGGTATAATTCACACTCCAGAGCTCCTCCTGGGACCAGGCTGCGGCT
GAAAATCTCCTGAAACACCTTCTGAGTGGCCATTTCCTCCTCCTGCCCCATCCTGCTTCCCTCCCTGCAAGGGTCTC
CTGAGAGCCCTCCCTCAACAAATGAGTCACATAAAATCCTCATCTCAGGCTTTGCTTCTCCAGAAATGAATGAAAAA
CAAGTGGCGATCCTTATTTTTGTGTTTCAGTTTTGTTTTGTTTTTTCAAATTTTGAAGGTCTCCTGTGGTGCAGTGG
ATTAAGGATCCTGTGCTGTCACTGCAGCGGCTCAGGTTGCTGCTGCAGTTGGGGAGTTCAAACCCTGACCCAGGAAC
TTCCGCATGCCATGCATGTGGCTAAAAAATAAAATGTTAATTGAAGGCACAAGGGAAAGAGCCAGGGTGGGAACCAA
GAGACCTGATGTTATCCCTTGTTCGGCCACCATCTCCTAGCAAGTGGCCAGCTGTGGTTCAACCTCCTGGGACACAA
GTCTCCTCCCCACCACATTGGGCATATGCATTTTCCTCGTGCAACTTACACTGTGCCATTGACTCCAACGGAGATAA
CGTGAATATTACCCAGCTGTAGAAACCACAACACCCTGTCGGAAAGAAAAGGAAAACACCATGAAACATCAAGAAGC
TCTTTAGATTCAACCTGAAAAATTACTTCTGGCACGGCTTCATGGAAACAGGTTTGGGGAGCCTAGATGAAAGCTGC
AGCTGAGTGATATACGTTGTTCAATATAATCTGCACAACAACCATTCCTGCTTTTCTGCATGTCACTTCTGTTTTTC
ATTCTGTTTATATTATCTTCATTTTCTTTTCAAAGAGTTCTAGCTGATTTTCAAAAATATGCATTTAAGTATGCGTC
CTCAAAGGGAACGACATCTCTCCTAAAAGGGCAAAACTGGAGTTCCCGTCATGGCGCAGTGGTTAACGAATTGGACT
AGGATCCATGAGGTTGCAGGTTCGATCCCTGGCCTTGCTCAGTGGGTTAACGATCTGGCGTTGCCGTGAGCTCTGGT
GTAGGTCACAGACATGGCTCGGATCCCGCGTTGCTGTGGCTCTGGCGTAGGCCAGCGGCTACAGCTCTGATTAGACC
CCTAGCCTGGGAACCTCCATATGCGGCAGGATCGGCCCTATAAGGGCAACACGACAAAAAATCAGAGAAAAAAAAAA
GGGCAAAACTTGGTTCTTGGGGAAAGATGAAAAACATTGTACTCTTTTATATACAAGACACATAGATATACATATAC
CATATAAATAAATACACACTATATCTGTAGTATTATTTTTTTTGGTCTTTTGTCTATTTAGGGCCGCACCCACGGCA
TTTGGAGGTTCCCAGGATAGGGGCTGAATCAGCTACAGCTGCTGGCCTCCACCACAGCCACAGCAACACCAGATCTG
AGCTGCAACTGTGACCTACACCACAGTTCACGGTAATGCCGGCCCCTTAACCCACTGAGCGAGGCCAGGGATCGAAC
CCGCGTCCTCATGGATGCTAGTCTTGTTCATGATGCTAGTCTTGTTCATTAACCACTGAGCCACGATGGGAACTCCT
GTAGTATTAATTTTTTTGGGGAGAGTAAGACAATTCATTTTTTTTAATGTCTAAAAGGCAGCCCAGTCCCCCGTATT
TAGTTCCTCTCCAACTACATCATCATCATCACCCTCATCATCACCATCATCTTCAGCATCACCATCACCAGTCTCAC
CAGCATCTTCACCACCACCATCATCATCCCCATCATTATCATCACTGCTATCAACCTCATCATTATCTTCAGCATCA
CCATCATCACCACCACCATCATCATTATCCCCATCATCATCATCACCATCACCAGTGTCATCACCACCACTCTTTGT
TTCTTGCGGGCAGAATAAAGAGTGCTAATGGCAGGGAGTTCCCGTGGCGCAGTGGTTAACGAATCTAACTAGGAACC
ATGAGATTGCAGGTTCGATCCCTGGGCTTGCTCAGCGGGTTAAGGATCCGCGTTGCTGTGAGCTGTGGTGTAGGTGG
CAGATGCAGCTCAGATCCCACATTGCTGTGGCTCTGGCGTAGGCCGGTGGCTACAGCTCCGATTTGACCCCTGTCCT
GGGAACCTCCATATGCCGTGGGAGCAGCCCAAGAAATGGTAAAAAGACAAAAAAAAAAAGAGTGCTAATGGCTAATC
CCAGTGCTGACACCCCCAAAGAAACAAGGCCACAATTCAGGATTTGGGGTCCACAGTCACCTGCTCTTTCTAATGAA
ACCTGCCACTCAACAAGTCTCACAAACCTAAACTTCCAACTTCCCTCAGTATCACTAATTGAAATTTCTCTTGCTCT
TTAGTTATTTTAGAGGCAACAGAGCATCATGTTTAAGCATATCAACTCTGACATCACATGTTTGGTGTCAAAATCTA
GCTTCACCAATTACAGACTGTGCGGCCTTGGGAAAGTTACTTAATTTCTTTGTGCCTATGTTTTCTCTTATGTGTAA
TAAGGGAAACAAATCCACTGTACAACAGCTGAGGAAACCCACACTTGTTGCTTAGAAAAGGTCTCCTATTCTTAGAT
TTGAACCAATGATGAAAACTCACAAGACCCATGAAGGGAACAATGACATGAAAAAAGCAAGACCAAGAAAAACTGAC
ACCTGAAGAAAAAGAAATAAAAGAACAGGAAAGGAGTTCTCATCTTGGAGCAGCAGAAATGAATCTGACTAGTGTAC
ATGAGGACGTGAGTTTGATCCCTGGCCTCGCTCAGTGGGTTAAGGATCCAGCGTTGCTGGGAGCTGTAGTGTTGGTC
ACAGATGCAGCTTGGATCCTGCATTGCTGTGGCTGTGGTGTAGGCCAGCAGCTGTTGCTCTGATTCAACCTCTGGCC
TGGGAACTTCCATAAGCTGTGGGTGCAGCCCTAAAAAGAAAAGAAAGAAAGAAAGAAAGAAAAGAAATACCTTCCCT
GGTTTCCTCCTTCTATATAACCCCCGATCACACTATACGACAGCTTCTTTCATAGCTCTTATCACCCCTGGAATGCC
CCGTTTTATATATTCTTCGGAGCAGCATAGTTTAGGAATAAAACATACAGACTCTGGAACCAGGCTGGCTTTAAAAC
CCTGGCTCTACTCCCTTATTACATAAGTGGTCTTGGGCAAGTTATTCAATTTCTTTTACCTCATTTTTTTCTCCTTT
GTAAAATGGGACTGTTTCAGGACCCAATATCAGAGGAATTTAGTGAAGACTGAATATGTTCTCTATTTGAGGAACTT
AGAACAGTGCTAAGTGAGTGGTTGCTATTACCGTTAGTGGCTTCCTTTCTGCCTACCTCTTCCTGCTGGTAAGTCAG
CCTCACAGGGCAGGAACTTTGTCTGTTCACTGCTCTATCCTCAGTGCCTAGAACGGCAGCTGGTACACGGTGGGTGC
TCAGAAAATACATGCCAAATGAAGGACTATAAAGAAATTCTTTCTTGGCAGATGAATTCCCTGATTTTTATCAAAGC
TTTCCTGATGAAGATGTTTGCAGTGTCCAGTCTAGAATTATGATCTCTTGGCTGGATAGCCCAAGGCCCTCCCTTTT
CCCTGCAGCCTATATCCAGTGTAATCTTCCCCCGGACTCCCTAGTCAGCCTCATACTCACCCCAAAAGAGAAGGAAA
CTGAAGCTCCACATCTTGCTGTGTTTCTGTCATTCGAAGAGGAGAATCTTTTCTCTGTTCCCAGAGTTTTTAATAAC
AGAGGGTGTGGAGAGAGGGGAAGGGCAGAGCCAGCATTGCTCAATGCAACCAGAGCATCACAGCCCTTTTTGCTGAG
TTGCCACCACTCGGAAAGGACAGTGTAGCAAACCCCTAATTTTCTCCTTTCTCCACAGTGTAGAGAGGTTGGTCTGG
CTGGTGGGTCAGTGTGTGGATCCATCTCCCTCTCTCTCTCTCTCTTTCCTTCCTGCTGGATTCTTTCTTTCTTTTTT
TTTTTTTTTTAATTGCAGCATAGTTAATTTACAATGTATACACATATATATTCTTTTTCAGCCTTTCCATTACAGGT
TATTATAAGATACTGAGTATAATTTACTGTGCTATATAGTAGGTCCTTGTTGTTTATCTCTTTTATATACAGTAGTG
TGTATATGTTAATCCCAAACTCCTCATTTATCTCCCCCTTCCACTTTGTTCTTTCCCCACCAACATCTATCTCCCAT
TTCTCATCATCTTATTTTATTGCACCCAGTAATAAATGAGCTTCCACCATCTATCCCCAATGAAGCAAGAGCAAAAC
TCAAGGGTCCTTTCCCAGTTTTCCCCGTACAATAACCACCATAAACCTCAAGTACCAGGCACTGTGCTAAATATGTT
TCCAAGAAAATTTAATTTCATCGCCATGTCAGCATCATCAAGTAGGGATTCCTACCCCTACCTATCTCATTTAAAAA
TACAATAGAATGGAAATTGCAACTACCAACCCCAAGCTCCCTGTCAACTATTACATTTAGAATGGATGAGCTAAGCA
ATGGGGTCCTGGCTGCACAGCACAAGGAAATATGTCCAGTCTCTTGGAATAGAACATGACGGAAGACAGTATGAAAA
AAAGAATGTATATACATGTATGTTTGGGTCACTATGCTGTGCAGCAGAAATTGATACAACGCTGTAAATCAACTACA
CTCTAATAAAAAATAAAGAAAGAAAAGTTAAAAATAAAGATGCTAGAAACAAAAAAGAAAAAAGGAAACTGAGGCTT
GGAGAGAAGATGTGTCTTGTCCAAGACTACCTGGACTTGAGATTTGAATCCAGGACCCTCTGACCCCAAAGACTAGA
ACTTTCACCATTTTGTTTGCCTTCAGCTCCCCATAATATCTGATCACTGTCGGTGACACTCCCACTCCATCCCCCCT
CCCCAAGCCCAACCGAAGACACACATACACATGCAACTTCTCATAAACAGGGTGGCCTAGGAATATCTTAGTTAGGG
TCTCCCAGATGCAGAGGCTGAGACAAGGCGTCTAGTGAAAGCAGTTCATCAGGGAGGTGACCCCAAAAACGCTCCAG
CTGAGGATGGGAGAAGTGAGAGAAGGAAGGAAAAGAGCCCACAATGAATGTTATCCAGTAAGTTACCCAGTAAAAAA
CTGAAACTGAAACAGAGGTTGAGGACATCTGTGCTATGTAGTAGGTCCTTGTTGTTTCTCTCGTTTATATGTAATTG
TGTGTATATGTTAATCCCAAACTACCTAAGAGACAGCCTAAAGCACCCTCTTCAGACTTATCCCAAACGAGGCGGGT
GAGGGAGCTGGGGTATTTATCCACCAGATGCTGTCGGTCACTGATTGAGGCTTGTGTTAACTTAAGACCTGGCCTCC
AAGCAGATAGAATGCGCTCCAGACCATAGCCCTGTTGATGACAAAATGCAGTGGCTGGCAGATGTCAGGCTAGGGCA
CCCAAATCCTGTGCTCCAAGATAAAACAGAAGGGCAAAGCCCAGCCCTGAGGTCTTGGGAAGAAGAGCCCCATTTGT
TTTCATATTCTCCTTTTTCGCTCTGGGCAAGGCAAAATACCTACCCTGGAATTATGGTCACCGAAGAAGATTCATCA
ACAGCTCCATCTGTGGATCAAGAGACCCTATCCAGTGAAGCTGCAGCTAAGAACGAGCACGAAAATACAGCAAAGCC
CTCCAAGAAGGAGGATAAACAGAGCTGTGTTACATTTAAGAGACACACTGGTGGATCAACACAGACCCTAGCACCAG
ATCGCAGGGGATTTAAATCCCGACTCCACCACTTGCTAGTCATATGCGGTCCTGGGCAACTTCTTAATGTCTCTATG
CCTCAACATTCCCATCTGTAAAATGGGGCTGATAAAAGGAGAATCTATTTCATGGAGTTAAGATGAGCATCAGAGGA
GTGGGTATATATCTCACGCTTAGAACCAAGCCTGGCACATAGAGAAAACTCCAAGATGTGGCTATTACTCAAATTCT
TTGATATTTCTCCCTTCCAGAGGGGGAACCCAGTTTTTCTCTCCTTGAATATGAGCTGGACTCAGTGACTTGCTTCC
AAGGAACAGGAAAAGGAAGATGTGACGTGTGGCCTCTGAAACATCTGAAAGTCATTGTGGCTTCCCCCTCGCTCTTA
CTTTCCAGGATCATTCAGTTGGGGGAAGCTAGTTATCGTATTGTGAGTTCACTCAAGCAGCGTGATAGAGAAGCCCT
CATGAGGAGGAACTGAGATTCCAGCCAAAACCTTGACTGTGACCTCATAAGACACTCTGATCCAGCCCCACCCAGCT
AAGCCACCTCTAGATTCCTGACCCTCAGAAACTGTAAGAAAATAAAAGTTTGTTGTTTGAAGCTGTTACATTTGGAG
GAGAGATGTGTTACACTGCAGGAGATAACTGATACGCTTAGAACCAATTGTCCTTGTCAATTAAAAAAAGGATAACA
ATAACATCATAAGAGTTTGAGGTTTGCTGGAATAAAACCTTAAAGTTCTACCTGGCAAAATAATGCCCACTAATATC
AGTAATTCTTGTTATTATTATTATCCCATTAGGCTAAGTGGTCACAGCTACTCATTGGCATCTGTTCCTGGGTACCA
GCAAGGACAGAAGTCAGCAACCCATTTCATGCAAGACCATCTAATGTGGGTGAGAAAGTTTAGACTTTCTCTGCTGG
GCAATAAAGGGATTTCAGCAAAGGAGTAACCATCCTGTTGGTAGTTTACAACACTCGTGTTGTGTAGACAGGATGTG
GTCATGGGTGGGGAGATGGGGAGAAGAACATAGCGACAAGCTCGTCTAGGGCACGGGTTGTGGAGACAGAGAGGAAT
TTAGGAAGCAGGAAAAGCAGAATGGGGGGAATGCATGCATGTGGGTGGGGGAGTCTAAAGCAGAAGGAGGAATTGAC
CTCTGGACATTGGGCTACAGAATTGAAAGTTCTTCCCATCCGGCCCAGGCTCCTTCTCGGGGTGGGATGGGATGGGA
TGAAATGGTGGAGGAGTTTTCCCGCTACTGCCAAAACAAATCGCCACAAACATATGGCTTGAAACAATACAAATGCA
ATACACGACAGGTCGGGAGGTCAGGGTCCCCGATGAGTCTTAGGAGGCTGAAATCAAGATATCCATGGGGGCTCCTA
GAGGCTCTGGGGAGAAGTCCATTCCCTGTCTTTGACAGCTTCTGGAGGATGCCCATATTCCTTCGCATTCCAAAGCC
CCTTCCTCCATCTGCACAGGCGGTGTAGTATCTCAAAATCTCTCTCCTCTCCCTCTCTCTCTCCTTCTCCCTCTTTC
TCCCTCTCTTTCACTCTCTCTCCCTTCCTCCCTCCTTCCCTCTCTCCCTCTCTTTCTCTCCCTCCCTCCCTCCTCTC
CCTCTCTCACACATACACACATACAAACACACACACATTTGCTCCATGGATGGATGGATGGATAGGTAGGTGGATTG
GTGGGTGGGTAAGATATAGATGGATCAATGGATGAATAAACAGGTAAGTAGATGTGTGTATTATGCTTTGATAGAGA
GAGAGAGAGATTGCTCTCATTCTCTAGATACATTTCTCTCATTCTCTCTATCCTCAATTTCTCTCTCTCCCCCACCT
CTCCCTCCCCTTTCCTCTCTGACCCTCCCTCCGCTCCCTTAAAAGGACTTTGTGATTCCATTAGACCTACTCAGATA
ATCCGCAATAATCTCCTATCTCAAAATCTTTAACTTCACTGCACTTGCAAAGCCCCCTTGGCAGTGTAAGGTATATA
TGTACAGCTTTCCAGGAGTGGGATATGGACAACCTGGTGGGAATTAGGGGGAATTTCATTATTCTACCTACTGAAGG
TGGGGTCTGGGGTCCTGGTGCGTGACTGAGGATGGCAAGATGCCAGTCACCCTTCAAATCCAAAAGAGGTGACCAAG
GCTATGAACTCTGGACCACAGAGATCCTCCAGGATGAGGGCAGGTAGCAGGCGTGAGGGGAGAAAAAAGGGAAGGAA
ATGCACAATTGGAGCCACATGGCTTGCAGAAGCCTAACCCCTTGTGACTTTCCCAGCAAAGAGGAAATTGAGAGATA
CTCAAGAAGTCATCTGAGGGTGTAATAGGAAAGAACAAATCTGACTCCATATTAGACCTGTTCCTTTTACTTTAACC
TTTGTGTCCTGTTGTTTTCCCTGAAAGAATGTTACCTAGAGCCTGAAATTCATCCCCCAGCCTGCATAGTCTCAAGC
CTCTGACCTTTAAGAGTATAACACGTTTCCATTCACATAGAGATAAAAAGTTGCAGAACAGAGAATTACATTTGTTT
TGTTGGAACCTTACAGGAACATCGGTGACCTGACCTATGCAGACAAAGGACTCCTGTACCAAGAAGGCTGCGACAAC
CAACCTGCCCTGCCCCACTTCCCCTGGCCTTTAAAAATGCTCTGCTGGGTATTCCCATTGTGGCTCAGTGGTAGCAA
ACGTAACTAGTATCCTTGAGGACTCTGGGGTTCGATCCCCCAGGCCTCACTTAGTGTGTTAAAGGATCCAGCGTTGC
TGTGAGCTGTGGGATAGGTTGCAGATGCAGCTCAGATCCTGCGTTGTTGTGGCAAAGGCTGGCTGCTATAGCTTGGA
TTCAACTCCTAGCCTGGGAACTTCCATGTGCCCTGGGTTCCGCCTGTGGAAAGTAACATAATGTCTTTTCTATCAAA
GGAAATCTTGGTTACTCCATTTTGCTCAGGTTTCACCTTCCTGCGACCCCCCCCACCCCTCCCCTTTCCCTCTTCTC
CCAATAACAATTTGTTTCAAATTAGCCAGCCGGGAAGAATGTGCACCCTGACCTGACCAATGGGAAGGGGACAGGTA
CATCACCTGCGTTAGGGATAAATAGGGGAGGGTCCTTTGTTCGGGGCGCACACTTTTTGGAGTGGCTGTGCCCTTCT
GCAGAAGTAAAGAGCCTTGTCGAGATTTCTCCTTGTCCATGTGTCTCACTTTCTGACACTGACGACCCAGCCCGAGC
TAGAGTTATTGGAATTTCCAACAGGCCTTAAAAAAAAAAAAAAAAAAAAAAAAAAAGACAATAAACATGCTTTGCTG
AAACCCTTTGGGAAGTTCCGGGTTTGGCAGTGGCGGGGGGAGGTGCATGAGGGCCCTTCCCCTCCAGCCCCCGCCCA
AGTCTCCTTGCACAGCCCTGCAATAAACCTCTCTCTGCTCCCAACTCCCCTGTTTTGTATAGTTTGGCCGCACTGAG
CAACAGGCACATGATCTGAGTTCGGTAACAGAGAAGCCCGGCCCCAGAGCATCCCTGGGTTCATGCTTAATGAGGGT
GTTGGAGGAAGGGCGGCTCCTGGGAAGCCCTCCCTACCCAACTGGACCGTGTTCCTCTCTCGTTCCCTCTAAACCCT
CCCCTGGCTCCCTGTGACCTTCGGGATGAAGTCCAGTCTCATTAATACGACACTCAAGACCTCACTGAGTCTTATAC
TGGTGCCCTTCTTCCTTATTGCCCCCCCTCACAAGTCCCAGTCATCCCAAATGAACCTGCAGTGCACACTGTCGCTG
ACCTGTCCAGCCATCCTTCAGCTACTGGAGCACCATCCCCCCGCTGCTGCGGGTGTTGCCTGCTAACAGTTCACAGC
TTCCCCTTCTCCAGAGAACGTTCCAGTTCAATGCCTGCATAAACCCTCAGGCCCATCCTGCAGCCAATAAGCAATGG
GCACAGGGGTCAAAAGCCAGCGTTCACCCCAAGGTGACTTCAACTTAGTGGTGTTATTCAGGCTCCGGGTGTTGGAA
ATTACAGTAACTCTGGCTCCGGTTGTCAGTGTTGGAAAGTGAGACACATGGACAAGGAGAAATCTCGACAAGGCTCT
TTACTTCTGCAGAAGGGCACAGCCACTCCAAAAAGTGTGCGCCCCGAACAAAGGACCCTCCCCTATTTATCCCTAAC
GCAGGTGATGTACCTGTCCCCTTCCCATTGGTCAGGTCAGGGTGCACATTCTTCCCGGCTGGCTAATTTGAAACAAA
TTGTTATTGGGAGAAGAGGGAAAGGGGAGGGGTGGGGGGGGTCGCAGGAAGGTGAAACCTGAGCAAAATGGAGTAAC
CAAGATTTCCTTTGATAGAAAAGACATTATGTTACTTTCCACACTACCCTTCCTCATCCTCTGCTAAATGTCCTCTC
TCAATAAACCCTGAAACAAACATCCTCAGGGCAGAGTCTGTTTCCAGGGGGACCTAAGAATCCCTCCCAGCCATTAA
ACTCTAAGCTGTCTCTTGACCTCAGGTTGCACATGGGTACTCACTCCATATTGTAGGCTTCCTTCCCATGTCAATAT
CACCTCCTCTTCCGTGCCTTCCTTTGTCAATCTCACCGCCTCTAGGAAGCCTTCCCACAAAAATATCACCTCCCCCA
GGGAGCCTTCCCATGTAAAATCACCTCCTCCAGGAAGCCTTCCCATAGAAATATCACCTCCAGAAAGCCCTCCCTGA
CCTCTCCTTCAGGATTAGGGACTTCTTCTATGCTTTCCTAATCCCAACACTTAATATGATCTTTGCTTGTTTCTGGA
TTTGGGGGTGGGGGTATGCTTGCTTTTGGTTTTTTCTGGGGTTTTTGGCCGCACCTGCTGCATACGGAAGTTCTCAA
GCTAGGGGTCAAATCAGGGCTGCAGCTGTCAGCCTACACCACAGCCACAGCAACGCCAGATCCGAGCCACATCTGCG
ACCTACACCACAGCTCATGGCAACACCAGATCCTTAACCCACTGAACGAGGCCAGGGATTGAACCTGCAGCCTCATG
GATGCTAGTTGGATTTGTTTCCTCTGGGCCACAACAGGAACTCCTGAAAAAACTAAAAATCTTAAAAAAAAAAAAAA
AGAAAGAAAGAAAGAACCAATGAGGAAAAAGAAGAAGGAACTGAAGAATCTCCTGACATCCCCCCCTAAGCCCTCAG
AACCAAGACCAAGAATGTAAGGGGATGGCCGATGGGCAGCCACTGCCCTCCCCCTGGAAGGAAGGAACACGAGTTCT
GCAAGGGGCAGCACTTGCTGAGGGGCAGAGTCCCAGCTTGCTGGGAAGGATGCATAGTTATCCAGGCTCCTAAGACC
CCTGGCAAGTGGAGAGGGGGGGTTGTTGAAATTCCCCTAGAACCACACCCAGGTCAAAGATTCCCCAGGATGGCTAC
ACAACTCAGTGCATAGCCATCCTCAGGCTGCTTTATTACAGCGAAAAGATACAAAGCAAAGACACAGAGGAAACCAA
AAATGAGGAAAGGGTTGAAATACATACAAGCTTCCAGCGGAGAGGTTCCCAGGAGAATGGAAGAAGCAGCCCCCATC
CATCAATTCCTTTTGCTGCGCTGATCTCGGTATGAGACTCCGACCCCAACCATCCTCTCCCGTTGTGTGATTTTTTT
CCTTTCCCCTATAATTTTCCCTGCCATGCCACCCCTCCCCCAAATTGTGTGACCTTCCTTTCATTGTCCTTGCCACA
AGTTCCCACCATGACCCTTTACAAGAGTAACATCTCAGGCGTTCCCGTCGTGGCTCAGTGGCTAACGAATCCGACTA
GGAACCATGAGGTTGAGGGTTTGATCCCTGGTCTTGCCCAGTGGGTTAAGGATCCGGCGTTGCCGTGAACTGTGGTG
TAGGTTGCAGACGCAGCTCAGATCCTGCGTTGCTGTGGCTGTGGTGTAGGCTGGCGGCTATAGCTCCGATGCAACCC
TTAGCCTAGGAACCTCCATATGCCGCGGGAGCAGCCCTGAAATGACAAAAAGAAAAACACTAAAGTCTCCTCACAGT
TGGAGCTGCTACTCTCTTGAGCTCAGCCCTTTGGTTCCGGAGGCCCTAATAAATCTCTCTTCTTGACTGACTTGGCC
TTGGGCGTTCTTCCTTCGAGCAAACCTAACACCAGGGTGGCCTGGAACCAGAGGGGCAGGGCGGGAGGGATCACAAG
AGAGCTCCAGAAAATTTAGGGAAACAATGGAAATGTTCCGTATCTTGAGTGTGGCCAAGGTTGCCAAACTCATCCAA
TTTTTACACTGAGAAACGAAGCAGTTTGTTGTATGTAAGTCACCCTCTCGTAAAATGGATAAGCTTGGCTCCAAAAT
AAAAGAGGACCCAGCATTCCATCAAATTATTTTCTTGTGCGTGCCACATGAAAGGACCCAGTTGTGTTATTGTGCAG
GCAATATATAAAGGGACCAGTTTATTTTATGCTATATAAAAGGGAACAAAAGATGGGCATTTTGAGTTTCTCCAGGG
AGGTGTGGGCTCTTTTACATTTAAACATTTGGGTTTTTTCGTTTTGTTTTTTTTTTTTTTTTGCTTTTCAGGGCCAC
ACCGGCGGCATATGGAGGTTCCCAGGCTAGGGGTTATTTCAGAGCTACAGCTGCCAGCCTACACCACAGCCACAGCA
ACACCAGATCCGAGCCGCATCTGCGATCTACACCCGACAGCTCACAGCAACACTGGATCCTTAACCCACTGAGTGAG
GCCAGGGATTGAACCCACGACCTCATGTTTCCTAGTCGGATTCGTTTCCACTGCACCATGATGGGAACTCCTAAACA
TTTGTTTAAATGGATAGCTTATCTTATTCCACAATAATAAATACATTTGACCTTAAGAAGCTTAGGAATGATCTAAA
TCTATACTTCCTTCAAATTAAAATGAAACCAAAAAAAAAAAAAAAAACTAGTACAGTTCACATTTCCTAACTGCACC
CTGACAGATAAGAAATGTTTCTTAGAATAATGCCATTTGCAGCAATATGGGTGGACCTAGAGATTATCATACTAAGT
GAAGTTAGTCAGAGAAAAACAAATGTCATATGATATCACTTGTGGAATCTCAAAAAATGATACAGAAAATTCCTTCG
TGATTCAGCAGGTTAAGGACCCAGCATTGCCACAGCTCTGGCATGGGTTTGAACCCTAGCCCGGCGAACTCTGCATG
CTGTAGTTGCTGCAAAAAAAAAAAAAAAAAAAAAAAAAAAATTAATTAAAAGAATATTTTAAATAAAGTGTTAAATG
ATATAAATTAACTTATTTACAAAGCAGAAATAGACTCACTGACATAGAAAACAAATTTATAGTTACCAAAGGGGATA
GTGGGGGTAGGGGGGAGATAAATTAGAAGTTTAAGGGTTAACATATACACATCACTATATATAAAATAGATCAGCAA
CGAAGACCTACTGTATAACTTAAACTATATTCAACATCTTGTCATAACCTATAATGGAACAGAATCTGAAAAAGGAT
ATATAAACATATTATATAAGTGAATCACTTTACTGTACACTTGAGACTAACACAACGTTGCAGATTAACTATACCTC
AATATTTTAATTTCACTCACATACCCTGCCCTGGGACTTACTAACTCTGACGAAGGCATCCACAGGTGATATTGGTG
GACATATTTCAAACACAGCCAGGCAGATATGGCATTGAATCAAACAGGGGCCTTTATAAACATCTCTTTCTCTCTTT
ATAAACATCTCTTTCTCTCTCTCTCTCCACCCCCCCAACACACTCTCAAACACGCGAGAGCGCTTTCCAACGCAGAT
AGCACCAAAGTAAAGCCAAGCTTGCCCTCTGGTGGACAGTATCAGTAGTGTCCCAAACTGCTGGGCTGATACTTGGA
TCCCAGCTTGGTGAAAGAAGTAGAGAGAGAGAGAGAAAGAGAGGGAGAGAGAGAAAGAAAGGTGTATCTGTGCACCT
GAGTTTGTTCACAAGCCTATATATATGAGCCCATATTTGGGCACCATAAAGGGCCCCTGATGCTTATGGCTTTGTAG
CATCCTCACACTGCCCAGTGGTATCTCCCATTCATTCACCCAAAAGCACAGAGAAGGGACTTATAGAGTCATTTCAG
AGTCTTGTTGGACACAAGCAGTCATAGCCTCATGTAGCCAGGATGGGGCAAGAGGTAGAAACACAGAGCTGGAGGAA
GCTAGAGGGAGAGTTTGGATCTAAGTCTCTGAAGGGTAAACATGGGCCTATACTGTTGCAAAGGCAGAGAAACCTAT
TGTAGATGGAGTGGGCTCTACTCAAAGCCTTTTACTGTAGCACAAAGCCTCTTCTTAATTCTTTAATCCCTTCCAGA
GGGCTAGGTTTGGGCTGTTGAGTTAGTACTTGGTATCTTCTAGAAGAGAAATGAGTGAGCCAAAGAAATGACTCTCT
AATGGTGGAATGACAATGAAGTCAGGCATAGGGCAGATTTTCTTTCTTTTTAAAAACAGTTTTTTGAGGTAAGACTG
AAACATACAAATTGTACATATTGAGTGTATACATAGCGATAAGTTTGGGGATACACATCCACTTGTGAGACCATCAC
CACCATCAAGGCCGTAAACATACCCATCACTTCTCAAAGTTTCCTTCTGCAGTGGATTTTCATTTTGGGGTCCCATC
ACATTTCATGGGGACTGTTGACTTGAGGAAAGTCTGTTCTCAGGGAGCCAGCACTCCTGTTTGAGTTGCGGGGGAGT
GTCTCAGGTCCCATGAAATATTTTCCCCGCTGCCTCCAAACTCATCAGTTTGAAGCTGTGTGCTGCTCTCTAGTGGC
CACCGCTCATTTGGCTCTAAGCTTTCCGCATAGATTGTTCTGGGACCAGACTGAAAGCGCAGGCTCCAAGTCAGGCT
TACAACTTTGAGCCTTAAATTGCAGGAGGTGGGGAGCCATGGATTAAGGAGACTTAATCAGTGGACAATTTGAGGTT
TTAATCAGTGTGGCCATTTCACACTTGACCTGGCAGATTTCCATTCATTAGTATCATCACTTGGTCTCCAGTCTCTC
CCATTTCCAATCTATTCTGCAAAAGCACAACCCAAGTCATATAGTCCAGGCAGCAGATTGAATCCTTAGGATGACCC
ACAGGGACTTATGTAATTTGCATCCTCCTCTTGATCTGGCTGCACTGACCTCTGGAAGCAGGAAAGGGCAGAAGAAA
AAGCTGAGCAAATATGCGGGCTCAGCTTGAGTTTACTTAGAATTAGTTTCATGGCGAAAATTAGTGTAGAGGAGCAA
GGTAGAGAGTATCTTGATGGTGGTGGGTGGTTATTATTGACTATGTGGTCAGAGAAGCCATCTTGGACATTTGAGCT
GAGACCTTGAGTGAATGGAGAGAGTGCCCAGGGAAAAGGGGGGAGGAGAAAATGTGTGTTAAGGCAGAGGGAATAGC
AGGTGCCAAGGCTCTGAGGAGGCTGTTGAGTCAGTACCCTGTATTTTCTGGAAGAGAAATTATTTAGCAAAAGAAAT
GACTCTCTAATAATGGAATTTTGGGCAATGAAGTGAGACAAGGAACAGATTTTCTTTTTCTGTTAAAAACCATTNNN
NNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNCCAGCCCGCCCCGGGCCCTGGGAGGGGAAGCCACCCGAACGCCTCAAACCTTTGCTC
TGGAAGCCCCAGGAATTTTTCCCCCTCTCCTAGCCGGGATATATGACCCCTCCTCTTTCTGGGGTGGTGGTAATCCT
GGGTTCCTGGGCGCCCTGGGGTAACTAGATAGCCCCTCGTGCCAACTCTGGGATTTCTTTTGGAGGTGCAGTGGAGT
CAGTGAGGGAAACCAAGTCACCCCTCGGGGGGGACCCGCGGAAGCATGGCGACCGGGAGAACCTGGTGCCTGCTCTC
TGGCCGTTCTGGGGGCCCCCCAAGCTGCGGGGAACCCTGTCCCTCTGGCCCTGACTCACGCCGGGCCGGCCGGATTT
TCCGGAATCTGGGGGGGATTAGGGGAGCCGGGGCAGGGGGAGTGGCCTTGCCCCATTCCACACCCCTGTTGGACGTC
TGGAGAGGGGACACTGTAGTCCGGCTGGGGCCCCGCCCCTGTTCCCTGGCCCTTCCTGGGAAGGGGAGGGGGTTCCC
GCCGGTTTCCTGCTTCCCCCCACCCCACGCCGCTCCGGGGCGGGGCCGGGAAGCCACTCCTTCTGGGAGCTCAGAGC
TTGGAGGCTCCCCTGGGCCAGGTCAGCGGGCTGTGGGGTCCCAAAGTCTTGATCCCGGTCCTCCCAATCCCCCGCTA
GGATCAGTTTGAGGTGCTTGAGCGGCACACGCAATGGGGTCTGGACCTGTTGGACAGATATGTGAAGTTCGTGAAAG
AGCGGACGGAGGTGGAGCAGGCTTATGCAAAGCAGCTGCGGTGAGACCCTAGGGTGGCCGCGCCCTGGGCTTCGGGG
GAGCGGTTGGAGGGCTGGGGGCTCAGTCTTCCTGCCTCTCTCCGTAGGAGCCTGGTGAAAAAATATCTGCCCAAGAG
ACCTGCCAAAGATGACCCTGAATCCAAGTAAGAATGAAGAGGGGAGGCAGAGTTAGATTTGGGAGGACTGGGGTATT
GGATCCTTTTCCTCTCCCTCCATTTGGGCCACCCAAGCACTCCTGGCTTCTCCACCCAGTTCCACTTAGAGGTATGA
GCTGGGAACCAGGAACCGTATTACCTGGGTTGGAATTCAAAATCCACTACTTTCTAGCTGAACTGCTTTGGGCAGTT
GACTCCAGTTCTCCGCCTCCATTTTTCTTGCCTATTAAATGGGAGAGGCTCCAACAGTTATTAAATGAATGACTCTG
AGCAAGTGACTTAAGTTTTGTGCCTCTGTCTTCCTCACTGTGAACTGGGGATGATGATCACAATACTGATCATAATG
ATAATGACCTTGTAGGGGCTCATTTGAAGATTAAGATAATGTGTTAAAACAATGCCCAGCCCATTTCACTTTATTCC
AAGCCCCCAGTTCCAGAATCCCCAAAGCTCTAAGAATCAGAAGCTTTTCTGGGCACCTATCCAGAGGCAACCTCTGA
CCTGAACTAATTTGACATTAATTACATTAATTGCGTTCTTGGTTTTTATCCCACTGAGTGTGAATGTTAATACTTAT
CATTGAGAGTTCCCGTTGTGGCTCAGCAGGTTAAGAATCTGACTAGTATCCATGAGGATATGGGTCAGATCCCTGAC
CTTTCTCAGTGGGTTAAAGCCCTGTGTTGCCATGAGCTGTGGTGTAGGTCACAGATGGGGCTTGGATCCTGCATTGC
TCTGGCTGGGGTGTAAGGCCTGCAGCTGCAGCTCCAATTTGACCCCTAGCCTGGGAACTTCCATATGCCTCAGGTAC
AGCCCTAAAAAGAGAAGAAAAAAAATCTCATACAAAAATGTTTATTAGATGCTGCCACTAACACCACTAGGGTAATG
TGAAAAGTGATATAAGCATCATATCCCCCTTCTGAACCCCCCTCAAAATCCTGAGAATTCTGAGTTCCCCCTCAGCG
GGTGGGGATAAGGGAGATTGGTTAGAATTTATCATTGCTTCTGGGTGAATGTTTTGGAGCTTACACTCTTCTGGGGC
ATATGGCTTCCAAGGGCCCTGACCCCTAGCCCCTGCCCCCTTCCCCCCACCCCAGGTTCAGCCAGCAGCAGTCCTTT
GTGCAGCTTCTCCAGGAGGTGAATGATTTTGCAGGCCAGCGGGAGCTGGTGGCTGAGAACCTCAGTGTCCAAGTATG
TCTCGAGCTGGCCAAGTATTCGCAGGAGATGAAGCAGGAGAGAAAGATGGTAGGTGATGCCCTCCTTGGGACTTCCC
CAGGGCCCTGGCCACCAGGCTGAGCCTTATTACCCCCTTCTTTCTGTAGCACTTCCAAGAAGGCCGCCGGGCTCAGC
AGCAGCTGGAAAGTGGCTTCAAGCAGCTGGAGAATGTGAGTTTGTGCATGGGGAGAAGAGGGGCACCCCTGAGCAGT
GGGGTGAGGGTGGCTGATCCATGGAGGTACCCCCTTGGTCTGGCCTGGTCCCCCACCTTCATTGTGGGTTTCCCCCT
CCATGTGCTGGGTGACTTCCCACCTGTCCCTGAAACCTTAGTTGGTGGCTCCTTCATGCCGGTCCTGTCCTCTACAC
AGAGTAAGCGTAAATTTGAGCGGGACTGCCGGGAGGCAGAGAAGGCAGCCCAGACAGCTGAGCGGCTGGACCAAGAT
ATCAACGCCACCAAGGCTGATGTGGAAAAGGTGCTTGTGCGGTCTGAGGCAGGCTTGGGGGGGGGGGGGGGCAGGGC
CCGAACCTGGCAGTGACCCCTGCTTTCATATTCCTCAGGCCAAGCAACAAGCCCACCTTCGGAGTCACATGGCAAAA
GAAAGCAAAAATGAGTATGCGGCCCAACTCCAGCGCTTCAACCGAGACCAGGCTCACTTCTATTTTTCCCAAATGCC
CCAAATATTCGATGTGAGTATTCAAAACCCACAGCCCCACCTCCTCCCCAAATTCTAAAATTAACCAACTCCTACAC
ATTTGTTGAAACCCCAGCTGCAATGCCCTAATCTCTAAATTGAAAGAGAATTAGAAATGAAGAGTCACAGTGCACTC
TGCCTTTTCTCAAGCTATTCGTTCTGCCCGGGTTGTCTTTCTTTCCTTTTAAAACTTCCATTTATTCTTTCAAGCCC
CATCAATTAACCCCTNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNTTCCTGCCTTCTTTGAAATCACCTCCGTTAACTATACCT
GACTCCCATGAGTGATTCTGCCATGCACATGTCCAATCTCTCTCTCTCCCCACAGTAGATAATCAATTCCAGGAGAA
CAAGTATTTGGGCCTGTATTTCTCACTGCTGCATCCTCCATCCCTAGAATCTGGGCGGGCATACAGTAGGTGCTCAA
CAAGTATTTTTGAATGAGTGCATGAATGAACGAAGAAATGAATGAATGATTATTGGCTTCAGCTTTGCAACTGAACT
CAGCTGAGACTCACTCGAACGCCTCTCCCACGAATGCTGTCTGTGAAAACAGATAGGACCTGATTCCCCCACAGACC
CCTGCACCTACCTCTACACATCTGTCCCGGGCCCTGGACACTCGTCTTTCCCCTGCTGGATTCAAATCCGGGCTTGC
AGACACAAGAGTAGCTCCCCACACTGTTTCGGCAAATCGCGTGCTCTGGGCAAGTTTTGGGATTGGCACATTCATTT
ACATCTAGTGAATGGGAATGAAAACCCGGGTCAAGGCAGAGGAAACAGTGAGGACAGGAAGCTGCGAACAGGACATT
CATCTCACCCACAAGGGTAGGAGCGAAGCATTCGAGGGACGGAACCCCCGTTACCCTCAATTACTGCCTTATCTACT
GCTTAGCTCCTAATAGACCCTCAACAAGAATTCAAATCCAAGTTTCTCTACTTGATAGTTATCTATCCTTATGCAAG
GGACTGTACCTCTCTGGGCCTCAATTTAATCATTTCTAAAATCAAGATCATAGACGCTACCCATAAGATCATCACAT
ATTACCTGTACAGATGAAACGACCTTTCTTTCCCAAGATCCAGTTGTTTCCAGTGGGAGATGAGAAACCAGTCAAAC
AGCTGCACCTGTACCTCCCTGGCAGGTCTTGCAGATTGAGTGAGGACCACATACTGGGGGGCTTTGAGAACACTCAT
CTATATCTGGACAGGAAAAGAGAGTCATAGTTGCCAATATGCTCCTTCATGTACAACAGATTGTATTTTTCAAAGAG
CTTGAAACACTGTCTTCCATCCCATGTGACCTGCATGCAATGTCCTTTAACTGGTACACTTTCCATCAAGCAGTGGG
TCTATATTTCCTTCCCTTGAATCTGAGTATGGTGGTGGGGACATTAGGTCATCAAAATACCATGGTAAATCATCAAA
ATATCATACACTTCCACCTTGTTCTCTTGAGATGCTCATGCTTGGCTCAGCCGCCATACTGTGAGGAAGCCTTGCAA
GTCATGAAAAACCAGCTCACACGGTAAGATTAAGACTTCCCACTCACAGCCCTGGAGCAGCCAACCAATAGCCAGCA
CCATCTTGAAGCCACAGGAGTGAGCCCCCTTCAAAGAGAATCCTCTAGCCCCCAGTTGAGCCAACACAACTCACACT
GTGGGGAACAGAGTTGAGCCATTCTCACCCAACTCAGCCCAAATAGCAGATTTATTTGTGAGCAAAATAAATGATTG
TTGCTGTCTTAATGTACCAACACCAATAGATAACCAGAACTTTTGCAAACCCACTTCTAGGAATTTACTCATTGGCG
CACCCATAGAATTGTGCAGCCATTGTACCATGGGGTGGGCCTCCCCAAATCTCCTTCAGCCCTGCTCTGCCAAGTCA
TCCTAAGTAAACATTTGCTTTGAAGTTGCTGGACAAATACAACTTCAAGGCAAGCGCCCTATAGCTCTCTTCCAGGA
AAATGCACCTCTCCAAGAGAGAAATCTGGACCTGCCACATGCATCAAGATAAGATCACAGGGATATTCTTCCCAGTT
TTAAGTAATGGAACATTAAACATCTAAATGTCTGTTGATAATAGGATGATTAAATCAGGAGTTGACATAAAGAATAA
TGTAGCATGTTCCTTCATTTGAGAAATATCTATTGAATATTCACAGTGTCTTAGGTACCATATTGGGAGTCAAAGAC
ATGCAGTGGACAAGGTCCTTACCATAGTATCCATCATTTTCTAGTTGGGGCATGTTGATTCTACCTGTATTTTATTT
TATTTTTTGCTTTTTATGGCCACACCCACAGCATATGGAGGTTCCCAGGCTAGGGGTCGAATCAGAGCTACAGCTGC
TGGCCTACACCACAGCCACAGCAACGCCAGATCCAAGCCACGTCTGTGACCTACACCACAGCTCACGGCAACACTGG
ATCCTTCACCCACTGAGCAAGGCCAGGGATCAAACCCACAACCACATGGTTCCTAGTCATATAATTTCTGCTGTTCC
ATGACAGGAACTCCTGATCCTACCTGTATTTTAAAACGAGGGACCAAAAAGACTACTGTGCTCACTGAATAATCCAT
GAACGATAGCCCAAAGGTTTAAAAAAGGATGTTTGGAGCTCCCTAGGAAATATAGTAATAGATATTAAATCATCTTA
TTCAGAGATTATCAAACTACAGCCCAAGTGTGAAATCTGGCCCACTACTTGTTTGTGTAAATAAAGTTTTATTGGAA
CACAGCCACATCCATTCATTTATGCATTATCTCTGGATGCTTTTGCATTACAACTGGAGTGTTGAATAATCAAGACC
TCCATATCATATGGCCTGCAACCTCCAAAATGTTTACTATCTGGCCCCTTGCAGAAAAATTTTGCGGATCCCTGGTC
TTATTCAGAAACATAGTCAGATCTTCACTGTTAAAAGGAAGTTTGGGTCTAAATATAAGGAATACATATCAAAAACT
AGCTCATTCTGGGTATTATTTTAGCTTATATTCTTTATGTTAACTGTAGCTCTTGGCACTCTACATGTGCCAAGCAG
GTTGTATACATTATTGCATTTAATTTTCCCAACTATCATTTAAGGTAAATACTTCTTTCTCTCTCTCTCTCTCCCTC
TCGGCCACCCTGTGCCATATGGAGTTCCTTCGTCAGATCCCAGCCACAGTTGCAACTCGCACAGCAGCTGTGCCACA
CCAGATCCTTAACCCACTGTGCTGGGCTGGGGACTGAATTTGCATCCCAGCCCTGCAGAGACGCTGAAGATCCGGTT
GCACCACAGCAGAACCCCTAAGGTAGATACTCACATACACCCATTTTATAGATGGAAATATTGAGGCTTAGAGATAT
TAATGATGTTTCTGCAACGCTTTACAACTGCTGTGTGGCAAACAGGTAATGTGGTTTGGAGACCGCCATTAGAGTCG
GAAAGTCCCGGGTTTGCATTCCAATTTAACTGCATGACTCTGAACACATCACTTCAGATATCCAAGCCTCAGGCTTC
TCATCTGTACAATGGAGGTCCTAGCAATGCCTATGCTCAATGTCATGTGAACAGGCACATAAAGCCCTTCACACAGG
GCCTGGCACTCCGTACAGGTTAGGAATTCATATTATTCACATGGAAGGAAATCAATGTCTATTTGGGGATATTGGCA
AATAGCATCTTTTTCTTTTTTTTCTAATGCAAGTCTCTAATCGCAAGAATTTTTGCTGGCCAGGTATCATTTCTCAT
AATCAAAACGCGTTGTCCCGGGCTAAATGTCTGCACCAGACTGNNNNACCNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNAGAA
GCCGAGAGCCGGGTCCTAAGCAACCGAGGGGACACCCTGGGCCGGCACACTCGGCCCCCAGACCCCCCAGCCAGCGC
CCCGCCAGACAGCAGCAGCAGCAACAACGGATCACAGGATAACAAGGAGAGGTGAGCAGGGAGGCCAGAGTGTGTGT
CTGCATCCAGGCCCAGGAGTGATGGGGAGGGGTCCTGTCCTCACCGGCTTTGCCCTCTCCAACCAGCTCTGAAGAGC
CCCCTGCAGAGGAGGGTCAGGATGCTCCCATCTACACGGAGTTTGATGAGGATTTTGAAGAGGAGCCCGCATCGCCC
ATAGGCCACTGTGTGGCCATCTACCACTTTGAAGGTAAGGACAGCCTGGGTGGCGCATCGGTGGCTTCGGGGATAGC
ATTTTTGGCTAGGCTCTGTTTAGGTTCACCTTGAGCAGATCTGAGCCCACCGCCACCCCCACCCCATGACAGGGTCC
AGCGAGGGCACCATCTCCATGGCCGAGGGCGAGGACCTTAGTCTCATGGAAGAGGACAAAGGTGACGGCTGGACCCG
GGTCAGGCGGAAACAGGGAGGTGAGGGCTATGTGCCCACCTCCTACCTCCGTGTCACGCTCAACTGAACCCTGCCAG
AGGCGGGAAGAGGGGGGGCTGTTGGCTGCTGCTTCTGGGCCACGGGGGGCCCCAGGACCTACGCACTTTATTTCTGC
CCCCGTGGCTTCGGCTGAGACCTGTGTAACCTGCTGCCCTCCCCCCCACCCTGCCCCGGAGCCCCCACTCAAGGGAC
CCACTGTGCCTTCCACCATCGATGTACATACTCATGTTTCCCATCTTTTCTTCCTGCCACTCGGCTGGGGCCGTTTT
GTTTTATATAAAACAATTATGAAAAGCTCTTACAGTCTGTGTCCTATTACGAGATTCTGATACTGGGGCTGGAGATT
CAAACACCACCCTCCCGACAGGTGGCACCAGGAAGGAGGAAGGGAAGGCGAACTTGGGCACACGTTGGCATCCCCTG
TCCCTTCCTGGGGGGTTGGGTGTGTTGATAGGGAGGAGGGTGCCAGATGTCACCCCTTTGGTGTTCTGCTATAGCTC
ACTGAGAACAGGTCACACCTGTTGAGCCCCTACTGTGTGCCAGGCATTTTCCACCCATGATCTCATTCAAACGCTGA
GCTTTAATCCCCATGACAACCCCTGGAAAGTACACAGTCTCACTTTTATGTTGAAGGCGGGGATAGAGAGAGAGGTC
AAGTGATCTGCTGGAAGTCACACAGCATTTAAAATGGATTTAAACTCTGGCCTCTTACAGATCTGCGAGTTCTCTTT
AACATTCAAAGCCTCACATTCACCACTTGTGGGATATGTTGAGGGGGGTGTGGGCATGGGGTGGTGAGAAAGGGCGT
TCAGAACCTCCAGATGTCGGGTCTTCTCATATGGGGAAGTAGGCTGCCCTCCCTTAGGATTCGTGCTCAGTTTTAGG
GTGCAGGGTGCGTTCTTGCAAACCAGGACCCGTCCCTTCTGTGAGGCTGGGTGCAGGTCCCACTGCATTTGGCTGCC
TGAGGACACTGGGGATCCCTGGAAGACTGGGTATCGCCGCGTGAAGAAGTGGATCTGTGCTTTCAAAGGTCAGGCTC
CAGGCGCTGCGACAGGACACTGAGGACGTGCTGGAACTTGTCGAAACGTGTGACCCACGGTGCCCCAGCCCCTCTGC
TTCCCCAGAGCAGCCTCCGCAAGAAACCGGTGGTCAGGGCCTCTTTCAGCTCAGGGTTGGGCTGGAATCCTGGGGGC
GGAGCCAGGTTAGCTGGAGGCGTGGCCAGGCACCTGCCTTACCCTCTGATAACTGCCTGGTCCCCTTGGGACTTTGA
CCCAGCAGGGGCCAGGAGGGATTCTGTCCCAGGTTATCTGAACTGCTGGGCAAGGTTAGCGGGGAGGGGGCTCCTGG
GTCTCTGCAGGGAGTGGGGTGGGGGTGGCTAACGGGCCCAGTGGAAGCGGGCTCTGCCAGGAGTGCATGGGAGCAGT
CTGCTCCAGGTGCAAGACCTGGTGGCCCCACCTTAGGGCTTGTGCCTGGAGATGGAGCTGCCCCGGGGGGCGGGACT
TGGGGTCCAGGCTACCCTACGCGACAAACGCCCAGGGGGTGGGGGTGGAGTTGGGCCTAGTTGGAGGGAGAAGAGTG
CTAAGTGAAGGCAGGAACTACCCAGGTGGGAGATTCTGGAAGCTGGGCTGCCCCAGAGAGGTGTGGCTCTGAGCTCA
GAGGGAGAGCAGTACCATCAGGTTGAAGGACTGAATCATTTTGGGGGGATCGAGATCCTCTGGGGGCAGGACCTTCC
CAAGTGTGAGAGAGTGGGACTCTGCGGGCGTGGCTCTGGGGGGATAGGGCCGCCCTTTAGGGGCGGACGGCACCATC
TGGTCTATTGCAGCATGCTTGAGTCGAGAAACACCCACCCAGGGCGGGGCCGTCTCAATTTGGGTGGGGCCCTCAGT
TTGGGAGGTGTAGCGGGAGGCTCTAGTCCCTGGGCCGGTGGGTTTGGGGGTGCCGGGCTACAGCATACGGCGTGTTC
TTAAAGTCAGGATCCTCTGGCAGCCGGGCGCAGATGGGGCGCTCACCTCGCAGGCGCCGGGCTGTCGCCTCGCGGAA
TCTGGGCGCGTCCCGGGCCGTCTGACGCGCCCGGTCCAGGGTGCGCAGCAGCCGCTCGCAGCTCTCGTCCAAGGGCC
CCGGGACTTCCTCGCCCTCCAGCAGGCGCACCACGGGTGCCACGTGCGGCAGCGCCACCTCGCCCGGGTCGCAAGGT
CCTGTTGGGGTAAGGGTCTGGGCTGGGCTAGGGGCAGAGGGTGGGTCCTAGGGTGAAGAGGGTGCGCCATACATTGG
GCCGAAACACCCTAGGACAAAAGCAAGGGGTGGAGTTTGGGTCAGATACGAAGTCTTGAGCAGGACCGAGTCAGGGG
AGGGGCCCAGGGAAGGGGCGGAGCCTAGGAGATAGTGAGGGCGGGGCCTAGGGATCTGGTCCTGGACCTAGCTTCGC
ACAGAGGGCGGGGGCTAGGGCGAGGGGGCGGGGCCTTGGACCCAAACAGCCAGCTGAATGTAGGGCGGAGGTGGAGC
AAAGGACAGAAACAAGGGGTGGATTTTGGTTGGAAAGAGACCCAGAGGCCAGAGAGCCTAGCAAAGAGCTGGATGGG
AGACAGGGACCAGGCCTAAGGCGCGGAGTGGGGTACTAAAACCCCCGCGGGGCTTAGAACCGGATTGAGGTCCTGAT
TAGAGTTACCACTTATCTAAAGATGCCACTCACCGGTGCCCTCATCCAGCGTCCGCATTAGAGGCTTCAGCTCCTGC
TCGAAGGCCAGCGCAGCCTCCGTGTGGCTCCTCCGGAGCTGGCGCCACGTGCGTTCCAACCGCGACACCTGGAAGAA
GAGACCCGAGCCCGCCTTCTCTCCACTCTTCCCCTTCACTTGCTCTCTGGCCTCGTCCCGCATCCCTGCTTCCTACG
CACCTGGGGCATGAGCAAGGCGCCCATGACCGCAGCCAGTCCAGGCAGGTCCCCCGCCGCCCCTGGCCGCAGCGCCA
GAGCCAGCTCCACCAGGCCCTTCAGGGCGGCGGCGCGCTCCTCCAGCGGTCCCGCGCAGCCCAGCACTGCCAGCGCC
CCCGCCAACGCCAGCGTCTCGAGCCTGCAGAGGCGGAGGGCAAGGTTTTGGAGGCAGTGGCGGGGTTTGCGATGTGG
GGGTGAGGAGGGAGAGCAGGTGACACAGCTCATCTCCCCTCCTCCCTGGGGGGCCGTGAATGGGGGGGAGGTTGAGG
ACCCTAGGGATTTTAGGTGGCTGCCTTACCTCTCCAGCAGTTCCAACCTCAGGCGATGTCCATGGGGAAGAGTGAGC
AGCTCCAGACCAGAGGCAACCCCCATGGCGCCCCGCTGAGCCTTGGTCACCCCCAGGAGGCCTGTCTCCTGGCAGTG
GGGGAGGGGTAAGAGTAGGGAGTTCAGAGGAGCAGAATGAGTAAATGGGTATGAGGTGAGACTGGGCCATGCCCTGG
CTTCAGCCCTACCTGGTAGGGGACCCACCTGGCAGTCCACCAATAGCAGGTGGAGGGCAGTGCTCCCAGGATGGTGC
TCCAGGAACAGGCCACGGAGAATGCGCAGGGCTTTAGGTTCCAGAGGCCGATTCTGGGGGCCCAGCAGGCAGGAGGG
GTTGTCAGGGGGACAGAAAGAGACCTCACCCTGTGGTCTTGCAAAACATCTTTGCTCCTCCTCCTCTTCCTCTTCAT
TGGCCTCCCACCATGGTGCCTCTGGCTCTGGGCAGCTGTGGCCAGGGGGTGTTCCATGGACCCTAGGCACTCGGGGC
ACCAGCTCACAGTACGTTGGGGAGCGTCCAGAGGCATCAGGCAGCATCAGTGAAGGTGTTCGAGGTGGCTTGGTTGG
TGCCTTGGCATGAAGCTGCCCATCGGAGGCCCTCAGGTTGTCAGCAATGGACCCCAGGAGAGCTGGGGTCTTTAGCA
ACACAGGGTCACTTCCTGTCCGAGGCAATGCAGATGCGGGCACAGTGGAGGCTCCTGGGGGTCACACAAGAGAGGGT
AAAAGAGGTCCACAGAGAAAATAGCTGGTTGGGGCTTTGTGGGGTACCAACCTCCAGGTTCATTCACTCGTTCAGCC
AATAACCAGGTACCCCACCTAACCTGGCGCAGCTTTGGACTTGAACGACTTCACCAAAACTCCTCTAGCCCATATGG
AGTTTTGCAAATTCTGAATGCTAAATTTTAAATTTGAATTCTTTGTTCTTGCCCCTCGGTTCCTGCATTGCTATAGC
TGTGGCATAAGCCAGCAGCTACAGCTCTGATTCAGCCCCTAGCCTCGGAACCTCCATATGCCGTGGGTGTGGCCCTA
AAAAGCAAAAATAAATAAATAAATACTCCCGTCTCCATACTCCTTACTCTGTTCTACTTTAGTTTTTGCCCCAAAGC
ATACATAATTGACTAATTTTTGTTGTTCATGGTCCTTCTTCCTCTAAAAGGATGTTGGCTCCACCAGCGCAGGGATC
TGTGTCATTCTTGTTCATTGATGTTATCACAGCACTTCATACAGTCTCAGGCATGTCACGAGACTTTGGGTGGAGGG
CAGGGCTGACAAGGCAGTCAGGACACAAAGGGGACTCTTCCTTTATGTAGGATTATCAGGGTCGGCTGCTCTGATGA
ACTCATGTTAGATGAGAAGGAGTCAGTCAGGTAAAGGTAGGGAATGAGCTTTTTCCAGTGGTGAGAATAGCAAGTGC
AAAGGCCCTGAGGCCGGAACATATTCGGCAGGTTCCAGCAACTGTAAAAAAGACTGTGTGATTGACGTGAAGAGGGG
ATGTAGCAGGAGTTATTAGGTCAGCCATGGTTAGAGCATATAGGGCTCCTTGGAATAATAACAAACCCACATTTTAT
TTTCTTCTTCTTATTTTTGGCCACACCCACAGCATGCAGAAGTTCTGGGGCCAGGGATGGAACCTGTGCCACAGCAG
AGACCTGAGCCGCAGCAGTGACAATGCCAGATCCTTAACTTGAGCCAATAGGGAACTCTGGAACTCCATAAACACAT
ATTTTTTTTTAAATTTTTTTACAAAGTTCCTGTGTGTTTTTAAATTACTGTGACAACATGAAGAGTATTACCATCCC
TTTTTTCCAAAAGGTTAAGTCCCCTGCCCAAGGTTCCTTAGGTATAGCCTGGCAGAGCCGTCCCTGAGCTCTGTGCT
GCCTGGGAAGCCCCTTACCTGGTCCAGGGTGGTCTTCTGTTGGGTGCCCCACATGCTCC

| | |
|---|---|
| SEQ ID NO: 27 | C3 cDNA Sequence |

CTCACTTCCCCCCCCACCCCCGTCCTTTCCCTCTGTCCCTTTGTCCCTCCACCGTCCCTCCATCATGGGGTCCACCT
CGGGTCCCAGGCTGCTGCTGCTGCTCCTGACCAGCCTCCCCCTAGCCCTGGGGGATCCCATTTACACCATAATCACC
CCCAACGTCCTGCGTCTGGAGAGTGAGGAGATGGTGGTGTTGGAGGCCCACGAAGGGCAAGGGGATATTCGGGTTTC
GGTCACCGTCCATGACTTCCCGGCCAAGAGACAGGTGCTGTCCAGCGAGACCACGACGCTGAACAACGCCAACAACT
ACCTGAGCACCGTCAACATCAAGATCCCGGCCAGCAAGGAGTTCAAATCAGAGAAGGGGCACAAGTTCGTGACCGTT
CAGGCGCTCTTTGGGAACGTCCAGGTGGAGAAGGTGGTGCTGGTCAGCCTTCAGAGCGGGTACCTCTTCATCCAGAC
GGACAAGACTATCTACACCCCAGGCTCCACGGTCCTCTATCGGATCTTCACCGTTGACCACAAGCTGCTGCCCGTGG
GCCAGACCATTGTCGTCACCATTGAGACACCTGAAGGCATTGACATCAAACGGGACTCCCTGTCATCCCACAACCAG
TTTGGCATCTTGGCTTTGTCTTGGAACATCCCAGAGCTGGTCAACATGGGGCAGTGGAAGATCCGAGCCCACTATGA
GGATGCTCCCCAGCAAGTCTTCTCTGCTGAGTTTGAGGTGAAGGAATATGTGCTGCCCAGTTTTGAGGTCCAAGTGG
AGCCTTCAGAGAAATTCTACTACATCGATGACCCAAATGGCCTAACTGTCAACATCATTGCCAGGTTCTTGTACGGG
GAGAGTGTGGATGGAACAGCTTTCGTCATCTTTGGGGTCCAGGACGGTGACCAGAGGATTTCATTGTCTCAGTCCCT
CACCCGTGTTCCGATCATTGATGGGACGGGGGAAGCCACGCTGAGCCAAGGGGTCTTGCTGAATGGAGTACATTATT
CCAGTGTCAATGACTTGGTGGGAAAATCCATATATGTATCTGTCACTGTCATTCTGAACTCAGGCAGCGACATGGTG
GAGGCAGAGCGCACCGGGATCCCCATCGTGACCTCCCCCTATCAGATCCACTTCACCAAGACCCCCAAGTTCTTCAA
ACCCGCCATGCCCTTCGACCTCATGGTGTATGTGACGAACCCCGACGGCTCCCCTGCCCGCCACATCCCGGTGGTGA
CTGAGGACTTCAAAGTGAGGTCCTTAACCCAGGAGGACGGTGTTGCCAAACTGAGCATCAACACACCCGACAACCGG
AATTCCCTGCCCATCACCGTACGCACTGAGAAGGACGGTATCCCAGCTGCACGGCAAGCGTCCAAGACCATGCACGT
CCTACCCTACAACACCCAGGGTAACTCCAAGAACTACCTCCACCTCTCGTTGCCCCGCGTGGAGCTCAAGCCAGGGG
AGAATCTCAATGTTAACTTCCACCTGCGCACGGACCCCGGCTACCAAGACAAGATCCGATACTTTACCTACCTGATC
ATGAACAAGGGCAAGCTGTTGAAGGTGGGACGCCAGCCGCGCGAGTCTGGCCAGGTCGTGGTGGTGCTGCCCTTGAC
CATCACGACGGACTTCATCCCTTCCTTCCGCCTGGTGGCTTATTACACCCTGATTGCTGCCAATGGCCAGAGGGAGG
TGGTGGCCGATTCCGTATGGGTGGATGTCAAGGACTCATGTGTGGGCACGCTGGTGGTAAAAGGTGGCGGGAAGCAA
GACAAGCAGCATCGGCCTGGGCAACAGATGACCCTGGAGATCCAGGGTGAGCGAGGGGCCCGAGTGGGGCTGGTGGC
CGTGGACAAGGGCGTGTTTGTGCTGAATAAGAAAAACAAATTGACCCAGCGTAGGATCTGGGATGTCGTGGAGAAGG
CAGACATTGGTTGCACACCAGGCAGTGGAAAGGACTTTGCCGGCGTCTTCACAGATGCAGGGCTGGCCTTCAAGAGC
AGCAAGGGCCTACAGACTCCCCAGAGGGCAGATCTTGAGTGTCCGAAACCAGCCGCCCGCAAACGCCGTTCCGTGCA
GCTCATGGAGAAAAGGATGGACAAACTGGGTCAGTACAGCAAGGACGTGCGCAGATGCTGTGAGCATGGCATGCGGG
ACAACCCCATGAAGTTCTCGTGCCAGCGCCGGGCTCAGTTCATCCAGCATGGTGATGCCTGCGTGAAGGCCTTCCTG
GACTGCTGCGAATACATCGCAAAGTTGCGGCAGCAGCACAGCCGAAACAAGCCCCTGGGGCTGGCCAGGAGTGACCT
GGATGAAGAAATAATCCCAGAGGAAGACATCATTTCCAGAAGCCAGTTCCCCGAGAGCTGGCTGTGGACCATTGAGG
AGTTTAAAGAACCAGACAAAAATGGAATCTCCACCAAGACCATGAATGTGTTTTTAAAAGACTCCATCACCACTTGG
GAGATTCTGGCTGTGAGCTTGTCGGACAAGAAAGGGATCTGCGTGGCTGACCCCTATGAGGTTGTGGTGAAGCAAGA
TTTCTTCATCGATCTGCGTCTCCCCTACTCCGTTGTGCGCAATGAGCAGGTGGAGATCCGAGCTATCCTCTATAACT
ACAGGGAGGCAGAGGATCTCAAGGTCAGGGTGGAACTGCTCTACAATCCAGCTTTCTGCAGCCTGGCCACCGCCAAG
AAGCGCCACCAACAGACTCTAACGGTCCCAGCCAAGTCCTCAGTGCCCGTGCCTTACATCATTGTGCCCTTGAAGAC
TGGCCTCCAGGAGGTGGAGGTCAAGGCCGCCGTCTACAACCACTTCATCAGTGATGGTGTCAAGAAGACCCTGAAGG
TCGTGCCAGAAGGAATGAGAGTCAACAAAACTGTGGTCACTCGCACACTGGATCCAGAACATAAGGGCCAACAGGGA
GTGCAACGAGAGGAAATCCCACCTGCGGATCTCAGCGACCAAGTCCCAGACACGGAGTCAGAGACCAAGATCCTCCT
GCAAGGGACCCCGGTGGCCCAGATGGTAGAGGATGCCATCGACGGGGACCGGCTGAAGCACCTCATCCAAACCCCCT
CCGGCTGTGGGGAGCAGAACATGATCGGCATGACGCCCACAGTCATCGCTGTGCACTACCTGGACAGCACCGAACAA
TGGGAGAAGTTCGGCCTGGAGAAGAGGCAGGAAGCCTTGGAGCTCATCAAGAAGGGGTACACCCAGCAACTGGCCTT
CAGACAAAAGAACTCAGCCTTTGCCGCCTTCCAGGACCGGCTGTCCAGCACCCTGCTGACAGCCTATGTGGTCAAGG
TCTTCGCTATGGCAGCCAACCTCATCGCCATCGACTCCCAGGTCCTCTGTGGGGCCGTCAAATGGCTGATCCTGGAG
AAGCAGAAGCCTGATGGAGTCTTCGAGGAGAATGGGCCCGTGATACACCAAGAAATGATTGGTGGCTTCAAGAACAC
TGAGGAGAAAGACGTGTCCCTGACAGCCTTTGTTCTCATCGCGCTGCAGGAGGCTAAAGACATCTGTGAACCACAGG
TCAATAGCCTGTTGCGCAGCATCAATAAGGCAAGAGACTTCCTCGCAGACTACTACCTAGAATTAAAAAGACCATAT
ACTGTGGCCATTGCTGGTTATGCCCTGGCTCTATCTGACAAGCTGGATGAGCCCTTCCTCAACAAACTTCTGAGCAC
AGCCAAAGAAAGGAACCGCTGGGAGGAACCTGGCCAGAAGCTCCACAATGTGGAGGCCACATCCTACGCCCTCTTGG
CTCTGCTGGTAGTCAAAGACTTTGACTCTGTCCCTCCTATTGTGCGCTGGCTCAATGAGCAGAGATACTACGGAGGT
GGCTATGGATCTACCCAGGCCACTTTCATGGTGTTCCAAGCCTTGGCCCAATACCAGAAGGATGTCCCTGATCACAA
GGATCTGAACCTGGATGTGTCCATCCACCTGCCCAGCCGCAGCGCTCCAGTCAGGCATCGTATCCTCTGGGAATCTG
CTAGCCTTCTGCGGTCAGAAGAGACAAAAGAAAATGAGGGATTCACATTAATAGCTGAAGGGAAAGGGCAAGGCACC
TTGTCGGTGGTGACCATGTACCACGGCAAGGCCAAAGGCAAAACCACCTGCAAGAAGTTTGACCTCAAGGTTTCCAT
ACATCCAGCCCCTGAACCAGTGAAGAAGCCTCAGGAAGCCAAGAGCTCCATGGTCCTTGACATCTGTACCAGGTACC
TTGGAAACCAGGATGCCACTATGTCAATCCTGGATATATCCATGATGACTGGCTTCTCTCCTGATACTGAAGACCTC
AAACTGCTGAGCACTGGTGTGGACAGATACATCTCTAAGTATGAGCTGAACAAAGCCCTCTCCAACAAAAACACCCT
CATCATCTACCTGGACAAGATCTCACACACCCTGGAGGACTGTATATCCTTCAAAGTTCACCAGTACTTTAATGTGG
GGCTTATACAGCCTGGGTCAGTCAAGGTGTACTCCTATTACAACCTGGATGAGTCTTGCACCCGGTTCTACCACCCC
GAGAAGGAGGACGGGATGCTAAACAAACTCTGCCACAAAGAAATGTGTCGCTGTGCTGAGGAGAACTGCTTCATGCA
CCATGACGAAGAGGAGGTCACCCTGGACGACCGGCTGGAAAGGGCCTGCGAGCCCGGCGTGGACTATGTGTACAAGA
CCAGACTTCTCAAGAAGGAGCTGTCAGATGACTTTGACGATTACATCATGGTCATCGAGCAGATCATCAAATCAGGC
TCCGATGAAGTGCAGGTTGGACAGGAGCGCAGGTTCATCAGCCACATCAAATGCAGAGAAGCCCTCAAACTAAAGGA
GGGGGGACACTACCTTGTGTGGGGAGTCTCCTCCGACCTGTGGGGAGAGAAACCCAACATCAGCTACATCATTGGGA
AGGACACCTGGGTGGAGCTGTGGCCTGATGGTGATGTATGCCAAGATGAGGAGAACCAGAAACAGTGCCAGGACCTG
GCCAACTTCTCTGAGAACATGGTCGTCTTTGGTTGCCCCAACTGATGCCACTCCCCCACAGTCTACCCAATAAAGCT
CCAGTTATCTTTCACATTTAAAAAAAAAAAAAAAAAAAAAAAAAA

| | |
|---|---|
| SEQ ID NO: 28 | C3 Protein Sequence |

MGSTSGPRLLLLLLTSLPLALGDPIYTIITPNVLRLESEEMVVLEAHEGQGDIRVSVTVHDFPAKRQVLSSETTTLN
NANNYLSTVNIKIPASKEFKSEKGHKFVTVQALFGNVQVEKVVLVSLQSGYLFIQTDKTIYTPGSTVLYRIFTVDHK
LLPVGQTIVVTIETPEGIDIKRDSLSSHNQFGILALSWNIPELVNMGQWKIRAHYEDAPQQVFSAEFEVKEYVLPSF
EVQVEPSEKFYYIDDPNGLTVNIIARFLYGESVDGTAFVIFGVQDGDQRISLSQSLTRVPIIDGTGEATLSQGVLLN
GVHYSSVNDLVGKSIYVSVTVILNSGSDMVEAERTGIPIVTSPYQIHFTKTPKFFKPAMPFDLMVYVTNPDGSPARH
IPVVTEDFKVRSLTQEDGVAKLSINTPDNRNSLPITVRTEKDGIPAARQASKTMHVLPYNTQGNSKNYLHLSLPRVE
LKPGENLNVNFHLRTDPGYQDKIRYFTYLIMNKGKLLKVGRQPRESGQVVVVLPLTITTDFIPSFRLVAYYTLIAAN
GQREVVADSVWVDVKDSCVGTLVVKGGGKQDKQHRPGQQMTLEIQGERGARVGLVAVDKGVFVLNKKNKLTQRRIWD
VVEKADIGCTPGSGKDFAGVFTDAGLAFKSSKGLQTPQRADLECPKPAARKRRSVQLMEKRMDKLGQYSKDVRRCCE
HGMRDNPMKFSCQRRAQFIQHGDACVKAFLDCCEYIAKLRQQHSRNKPLGLARSDLDEEIIPEEDIISRSQFPESWL
WTIEEFKEPDKNGISTKTMNVFLKDSITTWEILAVSLSDKKGICVADPYEVVVKQDFFIDLRLPYSVVRNEQVEIRA
ILYNYREAEDLKVRVELLYNPAFCSLATAKKRHQQTLTVPAKSSVPVPYIIVPLKTGLQEVEVKAAVYNHFISDGVK
KTLKVVPEGMRVNKTVVTRTLDPEHKGQQGVQREEIPPADLSDQVPDTESETKILLQGTPVAQMVEDAIDGDRLKHL
IQTPSGCGEQNMIGMTPTVIAVHYLDSTEQWEKFGLEKRQEALELIKKGYTQQLAFRQKNSAFAAFQDRLSSTLLTA
YVVKVFAMAANLIAIDSQVLCGAVKWLILEKQKPDGVFEENGPVIHQEMIGGFKNTEEKDVSLTAFVLIALQEAKDI
CEPQVNSLLRSINKARDFLADYYLELKRPYTVAIAGYALALSDKLDEPFLNKLLSTAKERNRWEEPGQKLHNVEATS
YALLALLVVKDFDSVPPIVRWLNEQRYYGGGYGSTQATFMVFQALAQYQKDVPDHKDLNLDVSIHLPSRSAPVRHRI
LWESASLLRSEETKENEGFTLIAEGKGQGTLSVVTMYHGKAKGKTTCKKFDLKVSIHPAPEPVKKPQEAKSSMVLDI
CTRYLGNQDATMSILDISMMTGFSPDTEDLKLLSTGVDRYISKYELNKALSNKNTLIIYLDKISHTLEDCISFKVHQ
YFNVGLIQPGSVKVYSYYNLDESCTRFYHPEKEDGMLNKLCHKEMCRCAEENCFMHHDEEEVTLDDRLERACEPGVD
YVYKTRLLKKELSDDFDDYIMVIEQIIKSGSDEVQVGQERRFISHIKCREALKLKEGGHYLVWGVSSDLWGEKPNIS
YIIGKDTWVELWPDGDVCQDEENQKQCQDLANFSENMVVFGCPN

| | |
|---|---|
| SEQ ID NO: 29 | MICA Genomic Sequence |

GTATCATTTCAGTGAAGGTCACTCCAGTCTTTCATGGAGGCCAAACTAAGGGTGTAAATTAGGATCCTCACTGAAGT
GGCGGGACCCTAAGAGGCTTTTTCCTGGCCCCTTAGTTGTGGGTTTTCCTGCGGGCGGCGCAGCCGGTTTCCATCAG
AACCGCCCAGAGGCGGACGCTGCCTTCCTGGGGTGACGGAGCAGCAGGAAGCGTTTTCGGATCCTGGAATACGTGGG
CGGCCCGTGGGAGGGGCTGAGGCGCAGTTTCCTACTCACCCGGATCCGAATCCTCCGCGGTGCTGTTTCAAGAGAGC
CGGATTCCAGATCACGCTCCAGCCCGGACTCGGAATTCCTGCCCTGCGGGTCTGCATTTTCATAACGGGCAGGTGTG
AGTGCCCTGCAGCTGGAGACCAGAAGCCTGAAGGCAGCTCGGCCCTCCCCAGCCCACAGCGCCGTTATTCCGTTTCT
ATATCAGTAAACACATTTCATTTTCCGTAGACCAGGGCGGGGTGACGGGTGATCCCAGTCCTCGCAGTGAATTCCGG
GCAGCAAAATTCAAAACACATGCGGCCAAGGCCGGGCACGGTGGTTCACGCCTGTAATCCCAGCACTTTGGGAGGTC
GAGGCGGGCGATCACCTGAGGTCGGGAGCTCGAGACCAACCTGACCAACATGGGGAAATCCCGTCTCTACTAAAAAT
ATAAAATTAGACGGGCTTGGTGGTGAATGCCTGTAATCCCAGCTAGTCGGGAGGCTGAGGCAGGAGAATCGCTTAAA
CCTTGGAGGCGGAGGTTGCGGTGAGCCGAGATCGCGCCATTGCACTTCAGCCTGGGCAACAAGAGGGAAAACTCCGT
CGCAAAAACTTTCGGGGGCGGAGCGGAGCCCCGCCCTGGGTTATGTAAGCGACCGCGCTGGGCCGTTTCTCTTTCTT
TTCCGGACCCTGCAGTGGCGCCTAAAGTCTGAGAGAGGGAAGTCGCCTCTGTGCTCGTGAGTGCATGGGGTATAAGG
CAAGTGCTGAGGGAGAAAACGTAGTTGATGGGGTAGAGCAGACGGGGTTGGAGGTGGGGTGGAGGGGGAGGGCTTTG
GACAGAAGACCTGGGAGGCTTGGTGGGGGAGGGGCGCCCAGGCCTGGGCACTAAGAAACAAGTCCCCTGGAGCTCAA
GACCATCTCGGCCTCCCCTAGCCCAAGAGAGGACTGGCTTCATGACTCCCTGAAACCATTTCTAAATGCCTTAGAAC
AAACCTTGCATATTCATTATTGTTATTGAACTATTAAAAGTCTTTTTTGGGGGCGAGCTGAATCAGATCCTTTGCTG
GAGCTGGCACACGGAGGAAGTCCTGGAGGGAGGGTAGACACCGTGGAGGTAAGGGCTTGGGACCTGTGTCAGGAGAG
CTAGGTCCATCTCCCTCCCAGTCTCTCACTAGGCTTATGATCTTTAGCAGTGAAAATAATCTCTCTAAGGTGGGGAA
AGGACCCCGGTCCCTGCTGTGCTCAATAAATTATGAGGATCAAAATAAATTATCAGTGAATGTGAATGGGAAAACTA
AGAAATTGTTAAAATTCTCGAATACATTACATTTTCATCCACAGAAAAGTGTAGGCTAGGGATCATGGGGGAATAGT
TAGTAATGACAGGGATAGTTGAACTTAAAAAAAAAGTTTGTGAGGCTGACAAAGAAGAAACGGACACATTTCCTGAT
CTTGGAGGGTTCATAGGGTAGAAGATGGTAGATGACAGCTGGGTGTGGTGGCACTCGCCTGTAGTCCCAGCTACTCA
AGAGGCTGTGGTGGGAGGATTGCTTGAGCCCAGGCATTCAAGGCTGCAGTGAGCTATAATCATGCCACTGCATTCCA
ACTGAGTGACACAGCAAGACTCCTCTCTTAAAAAAAAAAAAAAAATTCATGGCAGGGCACAATGAGTACTATCAGGA
AGGTTCAAACCACGGGCTAAATCAGTAGTTCTAAAACTTGACTACACATCGGAATCACCTAGGGAACTTTAAAAGAT
ACTAAGATTTAGGTCCAACCTGGGTTTACTGATTTAACAACCTAGGTTGTGGCTGTGGCCTGGGAACATGGATATTA
AAAACTCTCCAGGTGGTTCTACGCAGTGGCTAGGTTTGATGACCTCTGCCTAGATGTCCCAACGACTAAGAGATGTG
CGTTGGGGACAAGGCAATTCTCTTAGTAGAAAGAGGCTTTCGGGACAGCATTCTTATTATTGAGAATTGAGAATTCA
TATGCCACACAATTTATCCTTTTAAAGTGTGCAGCTCAGTGGCTTCTAGCGTAATCACAAGGTTGTGCCACCGTCAC
CACTGTCTACCCTGGAAGATTTTTTTTCCTTTTTTTCTTTTTTCTTTTCTTTTTATTTTAAAGGCTAGTCAAGTGAA
ACAGTGGGAGTGAAGAAGAAACAAAGACATCTATAACTGGTTGTGATCAATTAGTTGTAAACACTGCACTCAGACCA
GCCTGGGAAGATTTTAAGGATATGGTGTGGTCTGATGGGTTCCAAGGCAGAGGTTACAATAGCCTGGAAGAGGGAGA
CTGCTTAGGCAGTGGCATCCTGGTGGGATAGGGTGAGGAGATCCCAGAGCCCACGTTTACTGCAACCCTGGGGAGAT
GTCACCAGAGAAATGGGGGTGGTGCCAGACAGCAGATTGTGGCAGCTGAGGTTTTCCACGGTAGAGTAGAAGCATCC
ATCATGTGTGACATTCAGCAGATGGGGCGCTGTGGGTGGCTTGGAGCACTCTGGTTGTAACTGAGGCAGGCACCGTG
TTTAGGAAGGCTGTGCAGTAATCTAGGCTGAAGGGAGGGGAAAGCCTAGACTAAGATTGTGGCTGTGGGATTGAAAT
AGCGTTGAAGGAGCTGACTTTGACTCCCGGAGATGATGGGGAAAGAGGAAATCAGAAGGGACCAAGGATGGTGATGT
TCTTAAGAGAAACTGAGGAGGAAGAGAGGATGATATGGTGGCAGACGTATAGAGAGTCTTTGTAGATCTCTCACATT
GGAGGGGACTATGGTCGGAGGTACAGATGTCCTAAGGCAGGCTGGAAAAGGGAGTCTGGAGAGAGCTTGGTGTTGTA
GTGAACCACAGGGAGCCGCCTCCTTGGCCCTGTGATCACCCAGGGACTGAATAGAGAGGCGGCCCTGGGAGACTTCA
GACACTTAGAGGATATAAGGGGGTGAAAGGGGGGCCTGGCTTTGAGTCAAAGGGAGGAGAAGGAGATTATAAAGCTG
AAACGTCTAAGAGAGTTTGTGGTCTGAGCGGTTCTACTGCGGCAGGTGCTTCTGAGAGGCAGAGGTGGCTGAGATCT
GGAAACAGGTCTGCAAATCTGGTCACTGGTCTCATTGCCAGTAACGCTGTGCGCGGTTGAGGGAGTGTGTTGGGAGA
ATAGCCACGCGTTGTCTGTCCTGGAAGGAACAAGCCAGTGAGAGCCGGTTTAATGGGGCGGCCGGCGAAAGGGGCTT
GGTGAGGCCCGCGCTCCTCGGGGTGGGGGCGCGGGGATGGGTGGTCGCGATGCCGGGAGGGCAGGCAGGGCCCTGGC
CGTGCTTATGAAGTTGGAGCTGTACTCTCAGCTACTCGAAGCTGGTCCCTGCTTTAGGCTGCGCTCCCGCGTGCTCC
CCATTTTCTGGGCCCCAGGTCCCGCCTTCTAAATCTCCCCAGGTCTCCAGCCCACTGGAATTTTCTCTTCCAAGCGT
GGCCCCGCCCTCTCCGCTCGTGATTGGCCCTAAGTTCCGGGCCCCAGTTTCATTGGATGAGCGGTCGGGGGACCGGG
CCAGGTGACTAAGTTTCCGCGGCGCCTTCTCCCCGGCCACTGCTTGAGCCGCTGAGAGGGTGGCGACGTCGGGGCCA
TGGGGCTGGGCCCGGTCTTTCTGCTTCTGGCTGGCATCTTCCCTTTTGCACCTCCGGGAGCTGCTGCTGGTGAGTGG
CGTTCCTGGCGGTCCTCGGCGGAGCGGGAGCAGTGGGACGTTTCCGGGGGTCGGGTGGGTAGCGGCGAGCGCTGTGC
GGTCAGGGCGGGGCTCCTGTGCCCTGTCGGTGGCGCAGGGAGCTGGACGCGGCCCGTTACCGCCACACTTCAGCCCT
GCTTCCCCGTCACTTTTCAGTCCTCCTCGGGATCGCGCATCACCTGCACTTTCTGGTCTCCTCCTGCTCTTTCTCTC
CTCGCGTCTCCTCCGCTTCCTCTCACTTTTCGGACAAACCAGTCCTTCTGAGGCCCATGGGTTCCCGGGCTGCCTCC
GGGGCTGCTCCTGTGAATGGCATTCGAGTGCCCTTCCAGCGCGGCCACTGAAGCAGCCACAACCCCCGGTGCTCGGG
GCGGCTCTCAGGTCCCTGAAGTCCTGTCCTCTCCCGGAGCCGACGTGTTCTCAGCTCCTGGGCCGCAGCTCCTGGAG
TAGGGGCCCTCCTTTCTCGGGACCCGGAGCTGGTGCTTCCTGCTGCTGTGGGGACTGTGGGGGGTCCTGACTCTCAA
GCTGAGGGGTTGGAGTCTGCAGGCTCCGGGCAGAGGATTCTTCCTGCGACTTCTCTCATCCCCAGCTCATTCTCCCC
TCGCCTCTGGCTCCGAGGGTCCTCTCCTCTCTCTCATCCCACCCCTACTAATGACCAGTGATCTAAGGACACCAGAT
TCCCTCTCACCTCCTCCCTGCCCATCTCAGGGCCCGCTGAGTCCTTTTGCCCTCCCAGCTCCCTGCTACCCCTTCCT
GTGTGCTGTTCTCTGATCCATTTCTAGGGTGTCCTCTGCCCTCATCCCCTGTCCCCGCCACCGAAGGTCCCTCCTGC
ACCCCTTATGGGCCTTTCCTACAAGCAGCCTTCACCCAGTGCTGCCCCTATGCCTCCCCGTTCCCAAATGTCCCTGA
CTCTAACTTTCTGGTGCTGCCTTTTATCCGGGGGGGTCTTCCCTCCATCCCACTCCCCTCCAGACCCCCAAGGGGAA
CCCTGATGCTAATGGCAGTTGGGCCTTAGGCAGGGCGCAGGGCAGCGCAGATGCCCCCTCCCCTCCAGTGCAGATGC
CTGCTCTGGACCCTGCCTCATGGTGGCCCCTTCCCCACTCCTTCATCCTCAGCCTCACCCTCTTGAGGACCCCACCC
TCCAGCCCACAGGTGCTGGACCATCCCTCCCTGGTCCCTCCGCCCCTCTCCACCTTGGGACCTTGTGCTGCTCCTGT
CTCTTGCCCAGCTGCCTTGGGCCCTCAGCACGTTCTCATCTTTCAGTGGGAAAGTGGGAGTGCTGGAGCATATGACA
GTGCTGAGCATCTTTCCCAAGCCCCACCCTCCCCCAGAGCACCCTCCCCTCCTGTCCTCACCCTACCCCAAGTTCTC
CCACAGTCACTCCTGCCCCATGCTCATGCCGCCCTCCAGTTCTTGCTCTGCCCATCTCCCCTCCCCAACCCAGACCT
AAAACAGGCTGTTGGGCCAACTGTTCCTTGACCTTCCTTCTTTTCTTTTGGTTCCTTGACCCCAGTGGGCTCTCACT
CCCCACACCGCATATCTAAAATCTGTTTTGCCTGCTCTTGGGGTGCCACTGCTCCCCCTCCAGCATTACTCCTTTTG
GCAGGTCCTTCCTCAGGCTGAGAATCTCCCCCTCTACCTTGGTTTTCTCTCTCTGGCCAGCACCCCCACCCCTTGCT
TTGTTTTTAATTTTTAACTTTTGTTTGGGTACGTAGTAGATATATATGTATATATTTATGGGGTACATGGGATATTT
TGACACAGGCCTACAATATGTAATAATCACATCAGGGTAAATGGGTTATATCACAACAAGCATTTATCCTTTCTTTG
TGCTACAAACAATCCCATTATGCTCTTTCAGTTATTTTTAAATGTACAATAAATTATTGTTGACTGTACTCACCCTG
CTGTGCTATCTACTAGATCTTATTCATTCTAATTATATTTTTGTACCCATTATTAACCATCCCTGCTCCCCCACTCC
CCACTACCCTTCTCAGCCTCTGGTAATCATCATTCTATTGTCTCTCCCCATGAGGTCCATTGTTTTAAATTTTGGCT
GCCACAAATAAGTGAGAACATGCAAAGTTTGTCTGTCTGGGCCTGGGGCTTATTTCACTTCACAGGATGACCTCCAG
TTCTTTGCAAATGACACGATGGCTGAATAGTTCTCCACATACACATGTACACCACATTTTCTTTATCCATGCGTCTG
TTGATGGACACTTAGATTGCTTGCAGATCTTGGCTACTTTGAATAGTGCTGCAATAAACATGGAAAAGTAGATAGCT
CTTTAATATACCGATTTCCTTTCTTTGGAGTATATGCCTAACAGTGGGAGTGCTGGAGCATATGACAGCTCTATTGT
ATTTTTAGTTTTTGGAAGAACCTCCACATTGTTTCCCATAGTGGTTGTACTAGTTTACGTTCCCACCAACAGTGTAC
ATCCTCACCAGCATTCCTTATTTCTACATCCTCGCCAGCATTCCTTATTGCCTGTCTTCTGGATAAAAGCCAGTTTA
TCTGGGGTGGGATGTTATCTCGTAGGAGTTTTGATTTGCCTTCATCTGTTGACGAATGATGTTGAGCACCTTTTCAT
ATACCTGTTTGCCATTTATATGTCTTCTTTTGAGAAATGACTATTCAGATCTTTTCTCATTTTTAAATTGGATTATT
ATATTTTTTTTCCTATAGTTGTTCGAGCTCCTTATATGTTTCAGTTACTGATCCTTTGTCAGATGAATAGTTTGAAA
ATATTTTCTCCCATTCTTGGATGGTCTCTTCATTTTGTTTATTGTTTCCTTTGCTGTGCAGAAGCCTTTTTACTTGA
TATGATCCCATTTATGCAATTTTACTTTGGTTACCTGTGCTTGTGGGGTATTACTTTAAAAATCTTTGCCCAGTCCA
ATATCCTAGAGAGTTTCCCCAATGTTTTCTTGTATAGTTTCATAGTTTGAGGTCATAGATTTACATCTTTAATCCAC
TTTGATTTGATTTTTGTATATGGTGAAAGACAGGGTCTAGTTTCATTCTTCTGCATAAGGATATCTAGTTTCCCCAG
CACCATTTTTGAAGAGACTCTCCTTTGCCAATGTGTGTTCTTGGTACCTTTGTTGGAAATGAGTTTACTGTAGATGT
ATGGAATTGTTTCTGGGTTCTCTATTCTGTTTCATTGGTCTGTGTGTCTGTTTTTATGCCAGTATCATGCTGTTTTG
GTTACTGTAGCTCTGTAGTATAATTTGAAGTCAGATAATGTGATTCCTCTAGTTTTGTTCATTTTGCTCAGGATAGC
TTTATCTATTCTGGTTTTTTTGTGGTTCCATATGCATTTTAGGATTATTTTTATTATTTCTGTGAAGAATGTCATTA
GTGTTTTGATAGGGATTGCATTGAATCTGTAGATTACTTTGGGTAGTATGGATATTTCAACAAAACTGATTCTTCCA
ATCCATGAACGTGGACTATCTTTTCCATTTTTTGTGTCCTTCAATTTTTTGCATCAGTGTTTTTTGTTTTTGGTTTT
TGAGATGGAGTTTCACTCTTGTTGCCCAGGCTAGAATGCAAGGGTGTGATCTTGGCTCACCGCAACCTCCGCCTCCC
AGGTTCAAGCTATTCTTCTGCCTCAGCCTCCCAAGTAGCTGGGATTACAGGCATGTGCCACTGTGCCTGGCTAATTT
TCTATTTTTATTAGAGATGGGGTTTCTCTATGTTGGCCAGGCTAGTCTTGAACTCCTGACCTCAGGTGATCCACCTG
CCTCGGCCTCCCAAAGTGCTGGGATTACAGGCATGAGCCACCACGCCCAGCCACATCACTGTTTTATAGTTTTTATT
GGAGAGGTCTTTCACTTCTTCAGTTAGGTTTATTCCTCAGTATTTTATTTTATTTGTAGCTATTGTAAATGGGATTC
GTTTCTTGATTTCTTTTTCAGATTATTTGCTGTTAGCACTGATTTTTGCATGTTGATTTTGTATCCTGCAACTTTAC
TGAATTTGTTCTTCAGTTCTAATGGTTTTTTGGTGGAGTCTTTAGGTTTTTCCAAATATCAGACCACATGATCTGCA
AACAAGGATAATTTGACTTCTTCTTTTCCAGTTTTAATGCCCTTTCTTTCTTTCTCCTGTCTGATTGCTCTAGTTAG
GATCTGCAGTACTGTGTTGCATAACTGTGGTAAAATTAGTCATCCTTGTCTTATTCCAGATCTTAGAGAAAAGGCTT
TCAGTTTTCCCCCATTCAGTATGTTACTAGCTGTGAGTTTGTCATATATGGCTTTTATTATATTGAGGTCTGTTCCT
TGTATACTTAGTTTTTTGAGAGTTTTTATCATGAAGGGATGTTGAATTTATCAAATGCTTTTTCAGTATCAATTGAA
TGATACTGGCTTTTGTCCTTTATTCTGTTGATATGACGTATTACATTGATTGATTTGTGTATGTTAAATCATCCTTG
CATACCTGGAATACATTCCACTTGCTCATAAAGAATGATCTTTTTTAATGTATTGTTGAATGTGGTTTGCTAGTATT
TCCTTGACGATTTTTGCATCGGTGTTCATCAGGGATATAGGCCTGTAGTTTTCTTTTTTATGATGTGTCTTTGCCTG
GTTTTTGTATCAGGATATTCCTGGCTTTGTAAAATGAGTTTGGAAGTATTCCCTCCTCCTCTATTTTTCAGAACAGT
TTGAATAGGACTGACATATGTTGTTCTTTAAAAGTTTAATTGTGGTAAATTATACATTACATAAATTTTACTGTTTT
AACCACTTTTAAGTGTATACTCGGTGGCATTAGATACATTCACATTTTTGTGCAACCCAAAACTCTGTGCCCATTAA
TCGGTAACTCCCCATTCCTCCCTACCTCTGGCCCCTGGTAACCACCATTCTACTTTTTGTTTCTATGAATTTGACCA
CTCTAGGTACCTCATTTAAGCAGAATCATGTAATGTTTGTCTTTTTGTTTCTGGCTTATTTCACTTATAATATTTTT
GAGGTTCGGTGGGCACAGTGGCTCACGCCTGGATTTCCAGCACTTTGGGAGGCTGAAGCAGGTGGATCACCTGAGTT
TCGGAGTTCGAAACCAGCCTGGCCAACATGGTGAAACCCCATCTCTACTAAAAATAATAAAAGTTAGCCGGGCGTGA
TGGCGGGTGCCTGTAATCCCAACTACTTGGGAGGCTGAGGCAGGAGAATCGCTTGAATCCGGGAAGTGGAGGTTGCA
GTGAGCTGAGATCAGGCCACTGCACTCCAGCCTGGGCAACAAGAGTGAAATTCCATCTCCAAAAAAAAAAATAAAAC
AATAATAATAATAATATTTTTGAGGTTCATCCAAGTTGTAGTATGGGTCAGAATTTCATTCCTTTTAAGGATGGATA
ATACTCATTATATGTATGTACCACATCTTGGTTATCCATCCCTCAGACAATGGACACTTGGGTTACTTCTACCTTTT
GGATATTGGCAAATATTTCATTTCCTTTGGGTATATATTTATTTCCTTTGGGTATTTCTTTTGGGTATATATCCAGA
AATAGAAGCAGTACACAGGGGCTTCATTTTCTCTGTCTCTTTGCCAACCTTGCTCTGTGTGTGTGTGTATGTGTGTG
TGTAGGTGTGTGATAACAGCCATCCTGATTGGTTTCAGGTGGCATCTCATTGTGGTTTGGATTTGCATTTTCCTAAT
GAGTGCTGATATTGAGCATCTTTTCATGTGTTTGTTGATCATTTGTAATTTTCTTTGAAGAATTGGCCATTTAAGTC
TTTTGCCCATTTTTTCCCCCACATAGCTTCTCTTATCAGATATATGACTTGCAATATTTATTTCATTTCGGGGTTGA
TTGCTTTTTCACTCTGATTGTGCCCTTTGATGCATAGATGTTTTGAATTTTCATCAGTCTACTTTGTCAGTTCTTTC
TATTCTATCTGTGCTTTGGTGTCATATCCATGAAAGCACTGTCAAATCCTATGTCATGAACATTATCCCCAATGTTT
GCTTCTAAGAAATTTTTAGGTTTTAGTTCTTGAGTGTAGAGTTTAGGTCTTTGATTCATTTTGAGTTAATTTTTGTA
TATAGTGCAAATTAAGGGTCCAATTTTATTTTAACACCCCCTGCCCCCAGAACTATTTGCTGAAAAGATCAACTGAC
TCTTTGTCACCTGCTCACCCCAGTGGACACTAGCTGTTCCATCCAATTGCTGTCCTGGGGCCTTGTCATGCTACTCT
TCCACTTTGAACCCAAGCCCACACCGTTCGTTGCTCCCCTCTGGGATACTGACCCCACTATAAACTTCTCTGGGGCT
ACAACCTTCCTACCCTTTGTGCCTCATGACCACCCCCTCCCTTGTCCCCGCCATGCCCATGATGAGTCTCTTCTCGA
GGCAGCTCCCCTTGCCTCCATCTCACCCTCAGCCTATGCACCACAGCCACACTGGACATGGGTCCCTCTGAGCCTGA
GTCCCTTCCCATTCCCACCATCCCCTCTGGCAAGACCTTCCTTCCACCACCTTCATGCTCCTCCCTTGCCCCTGCAG
GGCAGCCTCTCCCCTTGGCCCCTATTCCCTTAGGGGGCTTGTGGCCACCCAGTCCTTGCACCTGGCCTACAAGTTTG
CCATCTTCATTCCCCCTTCTTCTGTTCATCAGCCCCCTCCTCTATCCTCCCACCCTCACAGTTTTCTTTGTATATGA
AATCCTCGTTCTTGTCCCTTTGCCCGTGTGCATTTCCTGCCCCAGGAAGGTTGGGACAGCAGACCTGTGTGTTAAAC
ATCAATGTGAAGTTACTTCCAGGAAGAAGTTTCACCTGTGATTTCCTCTTCCCCAGAGCCCCACAGTCTTCGTTATA
ACCTCACGGTGCTGTCCTGGGATGGATCTGTGCAGTCAGGGTTTCTTGCTGAGGTACATCTGGATGGTCAGCCCTTC
CTGCGCTATGACAGGCAGAAATGCAGGGCAAAGCCCCAGGGACAGTGGGCAGAAGATGTCCTGGGAAATAAGACATG
GGACAGAGAGACCAGGGACTTGACAGGGAACGGAAAGGACCTCAGGATGACCCTGGCTCATATCAAGGACCAGAAAG
AAGGTGAGAGTCGGCAGGGGCAAGAGTGACTGGAGAGGCCTTTTCCAGAAAAGTTAGGGGCAGAGAGCAGGGACCTG
TCTCTTCCCACTGGATCTGGCTCAGGCTGGGGGTGAGGAATGGGGGTCAGTGGAACTCAGCAGGGAGGTGAGCCGGC
ACTCAGCCCACACAGGGAGGCATGGAGGAGGGCCAGGGAGGCATACCCCCTGGGCTGAGTTCCTCACTTGGGTGGAA
AGGTGATGGGTTCGGGAATGGAGAAGTCACTGCTGGGTGGGGGCAGGCTTGCATTCCCTCCAGGAGATTAGGGTCTG
TGAGATCCATGAAGACAACAGCACCAGGAGCTCCCAGCATTTCTACTACGATGGGGAGCTCTTCCTCTCCCAAAACC
TGGAGACTGAGGAATGGACAGTGCCCCAGTCCTCCAGAGCTCAGACCTTGGCCATGAACGTCAGGAATTTCTTGAAG
GAAGATGCCATGAAGACCAAGACACACTATCACGCTATGCATGCAGACTGCCTGCAGGAACTACGGCGATATCTAGA
ATCCGGCGTAGTCCTGAGGAGAACAGGTACCGACGCTGGCCAGGGGCTCTCCTCTCCCTCCAATTCTGCTAGAGTTG
CCTCACCTCCCAGATGTGTCCAGGGAAACCCTCCCTGTGCTATGGATGAAGGCATTTCCTGTTGGCACATCGTGTCC
TGATTTTCCTCTATTGTTAGAGCCACTGGATAAAGACAGAGGGTCAGGGACTGGACCATCCAGTGTTGTAATCAGGG
CAAGTAGAGGACCCTCCGACAGAATCCTGAGCCTGTGGTGGGTGTCAGGCAGGAGAGGAAGCCTTCAGGGCCAGGGC
TGCCCCCTCTGCCTCCCAGCCTGCCCATCCTGGAGAGTTCCCTCCTGGCCCCACAACCCAGGAGTCCACCCCTGACA
TCCCCCTCCTCAGCATCAATGTGGGGATCCCAGAGCCTGAGGCCACAGTCCCAAGGCCCATCCTCCTGCCAGCCTGG
AAGAACTGGGCCCCAGAGTGAGGACAGACTTGCAGGTCAGGGGTCCCGGAGGGCTTCAGCCAGAGTGAGAACAGTGA
AGAGAAACAGCCCTGTTCCTCTCCCCTCCTTAGAGGGGAGCAGGGCTTCACTGGCTCTGCCCTTTCTTCTCCAGTGC
CCCCCATGGTGAATGTCACCCGCAGCGAGGCCTCAGAGGGCAACATCACCGTGACATGCAGGGCTTCCAGCTTCTAT
CCCCGGAATATCATACTGACCTGGCGTCAGGATGGGGTATCTTTGAGCCACGACACCCAGCAGTGGGGGGATGTCCT
GCCTGATGGGAATGGAACCTACCAGACCTGGGTGGCCACCAGGATTTGCCGAGGAGAGGAGCAGAGGTTCACCTGCT
ACATGGAACACAGCGGGAATCACAGCACTCACCCTGTGCCCTCTGGTGAGCCTAGGGTGACCCTGGAGAGGGTCAGG
CCAGGGTAGGGACAGCAGGGATGGCTGTGGCTCTCTGCCCAGTGTATAACAAGTCCCTTTTTTTCAGGGAAAGTGCT
GGTGCTTCAGAGTCATTGGCAGACATTCCATGTTTCTGCTGTTGCTGCTGGCTGCTGCTATTTTTGTTATTATTATT
TTCTATGTCCGTTGTTGTAAGAAGAAAACATCAGCTGCAGAGGGTCCAGGTGAGAAAAGCGGGCAGTTTCTGGAGAT
GGTAAGGCCCCTGTCTGGGCAGTAGGGTCCCCTCATTGCTCCTGCAAAGATAGGCATGTTGGTGACAAGGCTTCCAT
AACAGGGGATGAAAGTTGGGGAATTTGGGAAGGGAATGGGGGCAGCATCTCCATCTACACCCATAAGTGCTGCCCAA
GCAAGGGTCAAACGCCCAGCTGTGGCATCCTCCTGCTGCAGGTGAGGAGTGGGCAGCAGGGAGGGCTGCGGCGCCTG
CTCTGTCCCCATCCCGGTCTCTGTGTCTCTTGAACTCACTAGGGCGCATCCAGGTGGGGTGAGCTGGGAATCACGTG
CTGAATGCTAAGGGCCTGGATGATCACGGCCTCAGAGGGAGCAAATAGTAAAGGCAGCTGTGATCTGGGGAGGGCCA
GAAACTGGAGAGGAATCTGAGGAGAGGCGGTGCCCCTATTCCCTTCCTCTCTGCATCCCCCTCCCCTGTTTCTCCAG
CCATCGGGGCGGACACCGAGAAAAAGACCTATGAGGCCCAGCCTGGGGGCCCTGCCTGTGTAGCCCTTTGGAGACCC
CTTGTAACAGGGAGGGTCCTGAGCACACATGGCCATCTCTGTCCACTTTGCAGCTCCCCATGCACCTCCTCCAGGAG
CTTTCTTGGGGTTGTCGTGTCCTCTGCACCATTCGAGGCCCTACTCTTTCCAGGTTCCCACGGCCTGGCCTCCCTGA
GTTTCTTGCAGATGACATGGATGAGTAGATAAGCAGATGTCCCTGGGCCATTTGAGGAGTGGGGCCCAGCCCCTCAT
CAGGGCAGCTGTGGTCCCTGTTTTCATCCTACCTCCGAGTGTTTTCTTCTCCAGTCCCTGAGGGACACAGTCCTCAG
GGCCCATGTTTTTGGGGATTTAATCTGTGCTCTGTGGCCTCACCTTGCCCTCCCTGAGCCAATTTCCCTTTCTAAAG
GTGGTCACTGCCTGGTAAGTTTGGAGTAAGGGACGGTCAGAATCATTTCCCCTACAGTCAGGTTGTTTGATGGGGGA
TGAAAAGAGACAGCAGGAAGTTTTGTGTTTCTGCAAAGACAGAAGCAGTTCAGGCGACAGTAAGAGGCTGGGGTGTC
CAGGAGGATGTGTCTGGCAGTAGGGTCGCTGGTTTCTCATCCTTGAACCTAATTGCACTGTCAATCGGCCCCTCAGG
CCTGAGCAGATGGGAAGGTTTGTCCCCTGCCCTGCAGCAAGAGGGCCCTGTCCAGGAGGCACCCACAACAGGGGCAG
TGCAGGTCTGTGGTCACTCCTGCTCTCACCTGTGGCGTCTCCCGTAGAGGGATTGTCAGTTCTGGTTCCCTGTGGGC
AGGAATGGTTTCCTCATAGGTCACTGGAGTTTTGGCCAGGAAAAGAGTATGAAGTTCATGTGCCAGTTTCTCAAAAT
TCCTGCTTTCAATGTTGATGTCCAGTAAAGATATTCGTAATTTCAGCTCTATAATCTTAATAGGATTTCCTCTAATA
TTGTGAAGCATATTATATGAAACAGGAACACAAATTTCTCAAAATTCCTGCGATGTCCAATAAAGATTTTCATAATT
TCAGCTCTGCAATCTTAATAGGATTTCCTAATACTGTAAAGCATATTAAATGAAACAGGAACTCAAATTTGGAGCCC
CCTCTCCAGGAGGTTCTGTGTGGAGATGGTGGCTGTGGCAGTGGCAGTTCCCAGGTGCAGAGGGTGGGCAGAGGCAG
CCTCAGGCTAAGGGGTCTCCCCTACTCCACATGGAGAAAATCCCTTGTAGGTTGCAAGGGCAGTGGCCGGGTGGAAT
CCCTGCTAGGGACAGAGCAGGAAGGCCTCGCAGCCTCACCAAGCAGCAGCCCTGGGGTGGAGCTGCGTTTCCAGGGT
TAAGCGGACCAGGCAGGAGTAGCGGTTACTCAAGAGCAGGTCACAGGCTTGGGTTGTGAGGGTCAGGAGAGGCCAGG
CCTCCTCGAGCAAGGTGGGGGTCCCAGGGTCAGGTCAGGTGCAGATCCTGTGGCAGCCACGTCTTTCCATGCTGGGC
CTGCTGGGCCCCCCAGGCTTCCTGATGGGGTCCCCAGTTAGGAGCTGCCTGCTCAGGGCTGGGAGGGGAGGAGCACT
GAGCTGCAGATAGAGGGCAGAGCCCACAGTGGGCAGGGCCTGCCCTGGTGTGTAGGTGCCTCTGAAGGAGAGGAGGG
CCTGGGGACTGAGAGCAAGGGTCAGGGCCTCTCTTTGGGGAGGCCTCTCACTGTAACAGGACTGGTCAGGCCTGAGA
GGAGGGCACTGGGTTCCCTCTTGGGTCTTGTCCTTTAGTCTTGGGGCCCTTTCCCTCCCTGCACGATGAGTGGTGGG
CACAGGGCACGGGCTGATGTTGATGGAGTGATGGGAGGGAACTGGCAGGGGCTGGGAAAAGCAAGGAGGGAGGAAGA
AAAAAGTGGGGGCCTCATCTTCCCTCAGAGAAAGGGCAAATCTGGTTTTGGAGCAACTGAAGAGAGAAAAGTCCCCA
GGGAATAAACACAACACTGCACCCAGTGGAGCATTTACCCATTTCCCTCTTTTCTCCAGAGCTCGTGAGCCTGCAGG
TCCTGGATCAACACCCAGTTGGGACGAGTGACCACAGGGATGCCACACAGCTCGGATTTCAGCCTCTGATGTCAGCT
CTTGGGTCCACTGGCTCCACTGAGGGCACCTAGACTCTACAGCCAGGCGGCTGGAATTGAATTCCCTGCCTGGATCT
CACAAGCACTTTCCCTCTTGGTGCCTCAGTTTCCTGACCTATGAAACAGAGAAAATAAAAGCACTTATTTATTGTTG
TTGGAGGCTGCAAAATGTTAGTAGATATGAGGCATTTGCAGCTGTGCCATATTAA

| | |
|---|---|
| SEQ ID NO: 30 | MICA cDNA Sequence |

AAGTTTCCGCGGCGCCTTCTCCCCGGCCACTGCTTGAGCCGCTGAGAGGGTGGCGACGTCGGGGCCATGGGGCTGGG
CCCGGTCTTCCTGCTTCTGGCTGGCATCTTCCCTTTTGCACCTCCGGGAGCTGCTGCTGAGCCCCACAGTCTTCGTT
ATAACCTCACGGTGCTGTCCTGGGATGGATCTGTGCAGTCAGGGTTTCTCACTGAGGTACATCTGGATGGTCAGCCC
TTCCTGCGCTGTGACAGGCAGAAATGCAGGGCAAAGCCCCAGGGACAGTGGGCAGAAGATGTCCTGGGAAATAAGAC
ATGGGACAGAGAGACCAGAGACTTGACAGGGAACGGAAAGGACCTCAGGATGACCCTGGCTCATATCAAGGACCAGA
AAGAAGGCTTGCATTCCCTCCAGGAGATTAGGGTCTGTGAGATCCATGAAGACAACAGCACCAGGAGCTCCCAGCAT
TTCTACTACGATGGGGAGCTCTTCCTCTCCCAAAACCTGGAGACTAAGGAATGGACAATGCCCCAGTCCTCCAGAGC
TCAGACCTTGGCCATGAACGTCAGGAATTTCTTGAAGGAAGATGCCATGAAGACCAAGACACACTATCACGCTATGC
ATGCAGACTGCCTGCAGGAACTACGGCGATATCTAAAATCCGGCGTAGTCCTGAGGAGAACAGTGCCCCCCATGGTG
AATGTCACCCGCAGCGAGGCCTCAGAGGGCAACATTACCGTGACATGCAGGGCTTCTGGCTTCTATCCCTGGAATAT
CACACTGAGCTGGCGTCAGGATGGGGTATCTTTGAGCCACGACACCCAGCAGTGGGGGGATGTCCTGCCTGATGGGA
ATGGAACCTACCAGACCTGGGTGGCCACCAGGATTTGCCAAGGAGAGGAGCAGAGGTTCACCTGCTACATGGAACAC
AGCGGGAATCACAGCACTCACCCTGTGCCCTCTGGGAAAGTGCTGGTGCTTCAGAGTCATTGGCAGACATTCCATGT
TTCTGCTGTTGCTGCTGCTGCTATTTTTGTTATTATTATTTTCTATGTCCGTTGTTGTAAGAAGAAAACATCAGCTG
CAGAGGGTCCAGAGCTCGTGAGCCTGCAGGTCCTGGATCAACACCCAGTTGGGACGAGTGACCACAGGGATGCCACA
CAGCTCGGATTTCAGCCTCTGATGTCAGATCTTGGGTCCACTGGCTCCACTGAGGGCGCCTAGACTCTACAGCCAGG
CAGCTGGGATTCAATTCCCTGCCTGGATCTCACGAGCACTTTCCCTCTTGGTGCCTCAGTTTCCTGACCTATGAAAC
AGAGAAAATAAAAGCACTTATTTATTGTTGTTGGAGGCTGCAAAATGTTAGTAGATATGAGGCGTTTGCAGCTGTAC
CATATTAAAAAAAAAAAAAAAAAA

| | |
|---|---|
| SEQ ID NO: 31 | MICA Protein Sequence |

MGLGPVFLLLAGIFPFAPPGAAAEPHSLRYNLTVLSWDGSVQSGFLTEVHLDGQPFLRCDRQKCRAKPQGQWAEDVL
GNKTWDRETRDLTGNGKDLRMTLAHIKDQKEGLHSLQEIRVCEIHEDNSTRSSQHFYYDGELFLSQNLETKEWTMPQ
SSRAQTLAMNVRNFLKEDAMKTKTHYHAMHADCLQELRRYLKSGVVLRRTVPPMVNVTRSEASEGNITVTCRASGFY
PWNITLSWRQDGVSLSHDTQQWGDVLPDGNGTYQTWVATRICQGEEQRFTCYMEHSGNHSTHPVPSGKVLVLQSHWQ
TFHVSAVAAAAIFVIIIFYVRCCKKKTSAAEGPELVSLQVLDQHPVGTSDHRDATQLGFQPLMSDLGSTGSTEGA

| | |
|---|---|
| SEQ ID NO: 32 | MICB Genomic Sequence |

CTGTTTCCAGCGAGTCAGATTCCAGATCGCGCTCCAGCCTGGACTCGGAATTCCTGCCCCGCGGGTCTGCATTTTCA
CAGCGGCAGGTGTGAGTGCCGCGCAGCTGGAGACCAGAAGCCTGAGGCAGCTCGGCCCTCCCCAGCCCAAAGTGCCG
TTATTCCGTTTCTGTATCAGTAAACACGTTTCATTTTCCGTAGACCAGGGAAGGGTGATGGGTGATCCCAGTCCTCG
CAGTGAATTCCGGGCCACAAAATTCAAAACGCTTGCGGGCAAAGCCGTGCGCGGTGGCTCAAGCCTGTAATTCCAGC
ACTTTGGGAGGCCGAGGCGGGCGGATCACCTGAGGTCGGGATTTCCAGACCAGCCTGACCAACATAGAGAAACCCCG
CCTCTACTAAAAATACAAAATTAGCCGGGGGTGGCGCATGCCTGTAATCCCAGCTAGTCGGGAGGCTGAGGCAGGAG
ACTCACTTGAACCCGGGAGGCGGAGGTTGCTGTGAGCCGAGATCGCGCCACTGCACTCCAGCCTGGGCAACAAGAGC
GAAACTCCGTTTCAAAAAAAAACAAAAAACAAAAAGCTTTCGGGCGCCGAGGGCAGCCCCGCCCTGAATTTTGTGAG
CGACCGCGCTGGGCCGTTTCTCTTTCTTTTCCGGACCCTGCAGTGGCGCCTAAAGTCTGCGAGGAGGAAGTCGCCTC
TGTGCTCGTGAGTCCAGGGATCTAAGGCAAGTGCTGAGGGAGAAAACATAGTTGATGGGGCAGAGCAGAGGGGGCTG
GAGGTGGGGTGGAGGGGGAGGGCTTTGAACAGAAGACCTGGGAGGCTTGGTGGGGGAGGGGACCCAGGCCTCGGCGC
TGAGAAGCAACTCCCCTGGAGCTCAAGACCTTCTTGGCCTCCCCTAGCCCAGGGGAGGACTGGCTTCATGTCTCCCT
GAAACCGCTTCTAAATGCCTTAGAACAAACCTTAAATATTCATTATTATTATTGAACTATTAAAAGTCTTTTTTGGA
GGCGAGCTGAATGAGACCCTTTGCTGGAGCTGGCACACGGAGGAAGTCCTGGAGGGAGGGTAGACACCGTGGAGGGA
AGGGCTTGGGACCTGTGTCAGGAGAGCTGGGTCCATCTGCCTCTCTGTCTCAAACTATGCTTATGATCTTTAGCAGT
GAAAATAATCTCTCTAAGGTGGGGACAGGACCCCAGTCCCTGCTGTGCTTAATAAATTATGAGGATCAAAATAAATT
ATCAGTGAATGTGTATGGGAAGACTAAGAAATTGTTAAAATTCTCGAATACATTACATTTTCATCCACAGAAAAGTG
TAGGCTAGGGATGATAGGGGAATAGTTAGTAATGACAGGGATAGTTGAACTTAAAAAAAAAGGTTGTGAGGCCAACA
AAAAAGAAATGGACACAGTTCCTGATCCTGGAGGGTTCATAGTCTAATGGGGGAGGAGGGTAGAAGATGGTAGGTGA
TGGCTGGGTGTGTGGCACTCGCCTGTAGTCCCAGCTACTCAAGAGGCTGTGGTGGGAGGATTGCTTGAGCCCAGGCA
TTTGAGGCTGCAGTGAGCTATAATCACACCACTGCATTCCAACTGAGTGACACAGCAAGACTCCTCTCTTAAAAAAA
TAAAATAAAGTAAATGAAAAAAATAAGATTCAAGACAGGGCACAGTCGGTACCATCAGGAAGGTTCAAACCATGGGC
TAGATCAGTAGTTCTAAAACTTGACTACACATCGGAATCACGTAGGGAACTTTAAAAGATACTAAGGTTTAGGTCCA
ACCTAGGTTTACTGATTTAACTGGTTGTGGCTGTGGCCTGGGAACATGGATATTAAAAACTCTCCAGGTGGTTCTAC
GCAGTGGCTAGGTTTGAAGACCACTGCCTAGATGTCCCAATGACTAAGAATGTGCGCTGGGGACAAGCCAATTCTCT
TAGTAGAGGCTTTCCAGACAGAATTCTTATTATTGAGAATTGAGAATTCACATGCCACACATAATTTATCGTTTTAA
AGTGTACAGATCAGTGGCTTCTAGCATAATCACAAGGTTGTGCCACCGTCACCACTATCTACTTGGGAAGATTTTCT
TCCTTTTTTTCTTTTTTTTTTTTTTTTTTGAGGCGGAGCCTTGCTCTGTTGCCCAGGCTGGAGTGCAGTGGCGCAAT
CTCAGCTCACTGCAAGCTCCGCCTCCCGGGTTGACCCCATTCTCCTGCCTCAGCCTTCTGAGCAGCTGGGACTACAG
GTACCCGCCACCACGCCCAGCTAAGTTTTTTGTATTTTTAGTAGAGACGGGGTTTCACTGTGTTAGCAGGATGCTCT
CGATCTCCTGACCTCGTGATCTGCCCACCTCGACCTCCCAAAGTGCTGGGATTACAGGCGTGAGCCACCGTGCCCGG
ACCCTTTTTCCTTTTTTTTTTTTTTTAAAGGCTAGTCAAGTGAAACAGTGGGAGTGAAGATGAAACAAAAACATCTA
TAACTGGTTGTGATCAATTAGTTGTAAACACCACTGCACTCAGACCAGCCTAACTGGGAAGATTTTGAGGATATGCT
GTGGTCTGATGGGTTCCAAGGCAGAGGTGACAGTAACCTGGAAGAGGGAGACTGCTTAGGCAGTGGCATCCTGGTGG
GATAGGGTGAGGAGATCCCAGAGCCCACGTTTACTGCAACCCTGGGGAAATGTCACCAGAGAAATGGGGGTGGTGCC
AGACAATAGATTGTGGGAGCTATGGTTTCCATGGTAGAGTAGAAGCATCCACCATGTGTGACATTCAGCAGATGGGG
CGCTGTGGGTGGCTTGGAGCACTCTGGTTGTAACTGAGGCAGGCACAGTGTTTAGGAAGCCTGTGCAGTAATCCAGA
CTGAAGGGAGGGGAAAGCCTAGACTAAGACTATGGCTGTGGGATTGAAATAGCGTTGAAGGAGCTGACTTTGACTCC
CGGAGATGAAGGAGAAAGAGGAAATCAGAAGGGACCAAGGATGGTGAAGTTCTTAAGAGAAACTGAGGAGGAAGAGA
GGATGATGTGGTGGGAGACGTGTAGAGAGTCCTTGTAGATCTGTCATATTGAAGGGGACTATGGTCCCAGAGGTACA
GATGTCCTAAAACAGGCTGGAAAAGGGAGTCTGGAGAGAGCTTGGTGTTGTAATGAACCATGGGGAGCCGCCTCGTT
GGCCCTGTGATTACCCAGGAACTGAATAGAGAGGGGGCCCTGGGAGACCTCAGACACTTAGAGGATATAAGGGGGTG
AAAGGGGGGACCTGGCTTTGAGTCGAAGGGAGGAGAAGGAGATTATATAGCTGAAACGTCTAAGAGAATTTGTGATC
TGAGCGTTTCTACTGGGGCAAGTGCTTCTGAAAGGCAGAGGCGGCTGAGATCTGGAAACAGGTCTGCAAATCTGGTC
ACTGGTCTCATTGCAGTAACGCTGTGCGCGGTTGAGGGAGTGTATTGGGAGAAAAACCACGCGTTGTCTGTCCCGGA
AGGAACAAGCCAGTGAGAGCCGGCCTGATGGGAGGACCGGCGAAAGGGGCTTGGTGAAGCCCGCGCTCCTTGGGGGT
GGGAATGCGGGGATGGGGTGGTCGCGATGCAGGGAGGGCGACAGGGTCCAGGTCGTGCTCATAAGTTTGGAGCTGTA
CTCTCAGCTACTCGGGGCTGGTCCTTGATTTTGGCTGCGCTCGCGCACGCTCCCCCTTTTCTGGCCGCCAGGTCCCG
CCTTCTAAATTTCCCCAGGTCTCCAGGCCGCTAGAATTTTCTCTTCTGAACGTGGCCCCGCCCTCTCCACTCATGAT
TGGCCCTAAGTTCCGGGCCTCAGTTTTCACTGGATAAGCGGTCGCTGAGCGGGGCGCAGGTGACTAAATTTCGACGG
GGTCTTCTCACGGGTTTCATTCAGTTGGCCACTGCTGAGCAGCTGAGAAGGTGGCGACGTAGGGGCCATGGGGCTGG
GCCGGGTCCTGCTGTTTCTGGCCGTCGCCTTCCCTTTTGCACCCCCGGCAGCCGCCGCTGGTGAGTGGGGTTCCTGG
CGGTCCCCGGCGGAGCGGGAGCGGCGGGGCGTTTCCGGGGGTCCGGGTGGGTTGCCGCGAGCGCTGTGCGGTCAGGG
CGGGGCTCAGGTGTGCTGTCTGGAGTGCAGGGAGCTGGACGCCGCCTGTTCCCGCCACACCTCAGCCCTGCTTTCCC
ATCTCCCGTCTCTTTTTTTTTTTTTTTTTTTTTTTTTTTTTTTTTTTTTCTTTCTGAGACGGAGTCTCTGTCGCCTA
GGCTGTAGTGCAGTGGCGCGATCTTGGCTCACTGCAAGCGCCGCCTCCCGGGTTCACGCCATTCTCCTGCCTCAGCC
TCCCTAGTAGCTGGGACTACAGGCGCCCGCCACCACGCCCGGCTAATTTTTTGTGTTTTTAGTAGAGATGGGGTTTC
ACCGTGTTAGTCAGGATGGTCTCGATCTCCTGACCTCGTGATCCGCCCGCCTCGGCCTCCCAAAGTGCTGGGATTAC
AGGCGTGAGCCACCGCGCCCGACCTCCTGTCTCCTTTCAGTCCTCCTCGGGATCGCGCATCACCCGCATTTTCTGGT
CTCTCCTGCACTTGCTCTCCTCGCCTCTCCTCCGTCTCCTCTCACTTTTCGGACAAACCAGTCCTTCTGAGGCCCCT
GGGTTCCCGGGCTGCTCCTGTGAATGGCATTGGAAGGCCGTTCCAGCGCGGCCGCTGAGGCAGCCACTTCCCCCGGT
GCTGGGGGCGGATCTCAGGTCCCTGAAGTCCTGTCCTCTCCCGGAGCCGATGTGTTCTCAGCTCCTGGGCCGCAGCT
CCTGGAGTTGGGGCCCTCCTTTCTTGGGACCCGGAGGTGGTGCTTCTTGCTGCTGTGGGGACTGTGGGGGGTCCTGA
CTCTCAAGCTGAGGGGTTGGAGTCTGCAGGCTCCGGGCAGAGGATTCTTCCTGCGACTTCTGTCATCCCCAGCTCAT
TCTCCCCTCGCCTCCGGCTCCGGGGGTCCTCTCCTCTCTCGCATCCCACCCCTACTAATGACCAATGATCTAAGGAC
ACCAGATTCCCTCTCACCTCCTCCCTGCCCATCTTACGGCGCCCTGGGTCCTGTTGCTCTCCCAGCTCCCTGCTACC
CCTTCCTGTGTGCTGTTCTCTGATCCATTTCTAGGGTGTCCTCTGCCTTCATCCCCCGCCCCCGCCACTGAAGGTCC
CTCCTGCCTCCTTTATGGGCCTTTCCTGCAAGCAGCCTTCACTCCGTGCTGCCCCTATGCCTCCCCATTCCCAAATG
TCCCTGACTCTAACTTTCTGGTGCTGCCTTTTGTCCGGGGGGGTCTTCCCTCCATCCCACTCCCCTCCAGACCCCCA
AGGAGAGCCCTGATGCTAATGGCAGTTGGGCCTTAGGCAGGGCGCAGGGCAGCGCAGATGCCCCCTCCCCTCCAGTG
CAGGTGCCTGCTCTGGGCCCTGCCTCATTGTGGCCCCTTCCCCACTCCTTCATCCTCAGCCTCACCCTCTTGAGGAC
CCCACCCTCCAGCCCACAGGTGCTGGACCATCCCTCCCTGGTCCCTCCGCCCCTCTCCACCTTGGGACCTTGTGCTG
CTCCTATCTCTTGCCCAGCTGCCTGGGGCCCTCAGCAAGTTCTCATCTTTCAGTGGGAAAGTGGGAGTGCTGGAGCA
TATGACAGTGCTGAGAATCTTTCCCAAGCCCCACCCTCCCCCAGAGCACCCTCCCCTCCTGTCCTCACCCTACCCCA
AGTTCTCCCACAGTCACTCCTGCCCCATGCTCATGCCGCCCTCCAGTTCTTGCTCTGCCCATCTCCCCTCCCCAACC
CAGACCTAAAACAGGCTGTTGGGCCAGCTGTTCCTTGACCTTCCTTCTTTTCTTTTGGTTCCTTGACCCCAGTGGGC
TCTCACTCCCCACACCGCATATCTAAAATCTGTTTTGCCTGCTCTTGGGGTGCCACTGCTCCCCCTCCAGCATTACT
CCTTTTGGCAGGTCCTTCCTCAGGCTGAGAATCTCCCCCTCTACCTTGGTTTTCTCTCTCTGGCCAGCACCCCCACC
CCTTGCTTTGTTTTTAATTTTTAACTTTTGTTTGGGTACGTAGTAGATATGTATGTATATATTTATGGGGTACATGG
GATATTTTGACACAGGCCTACAATATGTCATAATCACATCAGGGTAAATGGGTTATCTATCACAACAAGCATTTATC
CTTTCTTTGTGCTACAAACAATCCCATTATGCTCTTTCAGTTATTTTTAAATGTACAATAAATTATTGTTGGCTGTA
CTCACCCTGCTGTGCTATCTACTAGATCTTATTCATTCTAACTATATTTTTGTACCCATTAACCATCCGCACTCCCC
CACTCCCCACTACCCTTCTCAGCCTCTGGTAGTCGTCATTCTATTGTCTCTCCCCATGAGGTCCATTGTTTTAATTT
TTGGCTGCCACAAATAAGTGAGAACATGCGAAGTTTGTCTCTCTGGGCCTGGGGCTTATTTCACTTCACATGATGAC
CTCCAGTTCTTTGCAAATGACATGATGGCTGAATAGTACTCCACATACACGTGTGCACCACATTTTCTTTCTCCATT
CGTCTGTTGATGGACACTTAGGTCGCTTGCAGATCTTGGCTATTTTGAATAGTGCTGCAATAAACATGGAAAAGTAG
ATAGCTCTTTAATATACCGATTTCCTTTCTTTTGGGTATATGCCTAACAGTGGGAGTGCTGGAGCATATGACAGCTC
TATTATATTTTTAGTTTTTGGAAGAACCTCCACATTATTTCCCACAGTGGTTATACTAGTTTACGTTCCCACCAACA
GTGTACAAGGGTTCTCTTTTGCTACATCCTCGCCAGGATTCCTTATTGCCTGTCTTCTGGATAAAAGCCAGTTTATC
TGGGGTGGGATGATATCTCGTAGGAGTTTTGATTTGCCTTCATCTGATGACGAATGATGTTGAGCACCTTTTGATAT
ACCTGTTTGCCATTTGTATGTCTTCTTTTGAGAAATGACTATTCAGATCTTTTGCTCATTTTTAAGTTGGATTATTA
GATATTTTTCCTATAGAGTTGTTTGAGATCCTTATATGTTTTGGTTACTAATCCTTTGTCAGATGAATAGTTTGAAA
ATATTTTCTCCCATTCTTGGATGGTCTCTTCACTTTGTTTATTGTTTCCTTTGCTGTGCAGAAGCTTTTTAACTTGA
TATGATCCCATTTATGCATTTTTACTTTGGTTGCCTCTGCTTGTGGGGTATTACTTAAAAAATCTTTGCCAGTCCAA
TATCTTAGAGAGTTTCCCCAATGTTTTCTTTCATAGTTTTCATAGTTTGAGGTCATAGATTTACATCTTTAATCCTT
TTTGATTGGATTTTTATATGTGGTGAGAGATAGGGTCCAGTTTCATTCTTCTGCATAAGGATATCTAGTTTCCCCAG
CACCATTTATTGAAGAGACTCTCCTTTGCCCTGTATGTGTTCTTGGTAACTTTGTTAGAAATAACTTCACTGTAGAT
ATATGGATTTGTTTCTGGGTTCTCTATTCTGTTTCATTGGTCCGTGTGTCTGTTTTTATGCCACTACCGTGCTGTTT
TGATTACTCTAGCTCTGTAGTATAATTTGAAGTCAGATAATGTGATTCCGCTAGTTTTGTTCTTTTTGCTCAGGGTA
GCTTTATCTATTCTGGGTTTTTTGTGATTCCATATACATTTTAGGATTGTTTTTCTATTTCTGTGAAGAATGTCATT
GGTGTTTTGATAGCAATTGCATTGAATTTGTAGATTGCTTTGGGTAGGATGGATATTTTAACAAAATTGATTCTTCC
GGCTGGGCACGGTGGCTCACTCCTGTAATCCCAGCACTTTGGGAGGCCGAGTCAGGTGGATCACTTGAGATCAGGAG
TTCAAGACCAGCCTGATCAACATGGGGAAACCCCGCCTCTACTAAAAATACAAAATTAGCCAGGCGTGGTGGCATAT
GCCTGTAATCCCAGCTACTCAGGAAAGCTGAGGCAGGAGAATCGCTTGAACCCAGGAGGCAGAGGTTGTGGTGAGCT
GAGATTGCACCATTGCACTCCAGCCTGGGCAACAGGAGCAAAACTCCATCTCAGAAAATAAAAATAAACATTGATTC
TTCCAGTCCGTGAACATGGAATGCCTTTTCCATTTTTTGTGTCCTCTTCAATGTTTTGCATCAGTGCTTTATAGTTT
TTATTGGAGAGATCTTTCACTTCTTCAGTTAAGTCTATTCCTAGGTATTTTATTTTATTTGTAGCTAATGAAAATGG
GATTCGTTTCTTGATTTCTTTTTCAGATTATTTGCTGTTAGCACATAGAAGTGCTATTGTTTTTTGCATGTTGATTT
TGTATCCTGCAACTTTACTGAATTTGTTCTTCAGTTCTAATAGTTTTTTGGTGGAGTCTTTAGGTTTTCCAAATATC
AGACCACATGATGTGCAAACAAGGATAATTTGACTTCTTCTTTTCCAATTTTGATGCCCTTTATTTCCTTCTCCTGT
CAGATTGCTCTAGCTAGGACTTGCAGTATTGTGTTGCATAACTGTAGTGAAAGTAGTCATCCTTGTCTTGTTCCAGA
TCTTAAAGAAAAGGCTTTCAGTTTTCCCCCATTCAGTATGTTACTAGCTGTGAGTTGTCATATATGGCTTTTGTTAT
ATTGAGGTCTGTTCCTTGTATACTCAGTTTTTTTAGAGTTTTTATCATGAAGGGATGTTAAACTTATCAAATGCTTT
TTCAGTATCAATTGAAATGGTGATATGGCTTTTGTCCTTTATTCTGTTGATACGATGTATTACATTGATTGATTTGT
GTATGCATACCTGGAATACATTCCACTTGGTCATGAAGAATGATCTTTTTAATATACTGTTGAATGTGGTTTGCTAG
TATTTCATTGATGATATTTGCCTCAATGTTCATCAGGGATATAGGCCTGTAGTTTTCTTTTTTTGATGTGTCTTTGC
CTGATTTTGATATCAGGATATTCCTGGCTTTGTAAAATGAGTTTGGAAGTATTCCCTCCTCCTCTGTTTTTCAGAAC
AATTTGAATAGGACTGATATTTCTTGTTCTTTAAACGTTTAATTGTGGTAAATTATACATTACATACATTTTACTGT
TTTAACCGCTTTTAAGTGTATACTCGGTGGCATTAGATACATTCACATTTTTGTGCAACCCAAAACTCTGTACCCAT
TAATCAGTAACTCCCCATTCCTCCCTACCTCTGGCCCCTGGTAACCATCATTCTACTTTTTGTTTCTATGAATTTGA
CCACTCTAGGTACCTCATTTAAGTAGAATCGTGTAATGTTTGTCTTTTTGATTCTGGCTTATTTCACTTATAATATT
TCGAGGTTCATCCAGGTTGTAGTATGGGTCAGATTTTCATTCCTTTTAATGATGAATAATACTCATTATATGTATGT
ACCACATCTTGGTTATCCATTCCTCAGACAATGGACACTTGGGTTACTTCTACCTTTTGGATATTGGCAAATATTTC
ATTTCTCTTGGGTATATATTTATTTCTTTTGAGTATTTCTTTTGGGTATATATCCAGAAATAGAATTGTTGGATCAT
ACGGTATTTCATTTTTTAATTTTTAGAGGAATCACCATAGTGTTTTCCATTGCAGGCGTGCCATTTTGTATTTCTAG
AAGCAGTATACAGGGGCTTCAGTTTCTCTACCTCCTTGCCAAACTTGCTGTGTGTGTGTGTGTGTGTGTGTGTGTGT
GTGTGTGTGATAATAGCCACCCTGATTGGTTTGAAGTGGTATCTCATTGTGGTTTGGATTTGCATTTTCCTAATGAG
TACTGATATTGAGCATCTTTTCATGTGTTTATTGATCATTTGTATATTTTCTTTGAAGAATTGGCCATTGAAGTCTT
GCCCATTTTTCTCCCCCACATAGCTTCTCATGGCTATTTTGCCCATTTTTGAGTGGGTTGACTGTTTTGTTGTTTTT
GTCAAACTTTTTTGCATATTCTGGAAACTAATCTCTCTCTTTTTCTTTTTTTTTTTTTTTTTTTTTTTGAGATGGAG
TCTTGCTCTGTTGCCCAGGCTGGAGTGCAGTGGCACGATCTCAGCTCACTGCAAGCTCCGCCCGCTAGCTTCATGCC
ATTCTCCCGCCTCAGCCTCCCGAGTAGCTGGGACTACAGGCGCCCGCCACCACACCCGGCTAATTTTTTGTATTTTT
AGTAGAGATAGGGTTTCACCATGTTAGCCAGGATGGTCTCAATCTCCTGACCTGGTGATACACCCGCCTCGGCCTCC
CAAAGTGCTGGAACTACAGGCTTGAGCCACCACGCCTGGCCTTCTGGAAACTAATCTCTTATCAGATATATGACTTG
CAATATTTATTTCATTTCAGGGGTTGATTGCTTTCTCACTCTGATTGTGCCCTTTGATGCACAGATATTTTGAATTT
TTCATGAGTCCAGTTTGTCAGTTCTTTCTATTCTATCTGTGCTTTGGCGTCATATCCATGAAAGCACTGTCAAACCC
TATGTCATGAACATTATACCCAATGTTTTTTTCTAAGATATTTTTATGTTTTAGTTCTTGAGTTTAGAGTTTAGGTC
TTTGATTCATTTTGAGTTAATTTTTGTATATAGTACAAATTAAGGGTCCAATTTTATATTATTTGAACATCCAGTTC
CCCCAGCACTATTTGCTGAAAAGATGGACTTACTCTTTGAGACCCTGTCACCTGCCCACCCCAGTGGACACTAGCTG
GTCCATCCAATTGCTGTCCTGGGGCCTTGTCATGCTACTCTTCCACTTTGGACCCAAGCCCACATCATTGCTCCCCT
CTGGGATACTGACCCCACTATAAACTTCACTGGGGCTACAACCTTCCTACCCCTTGTGCCTCATGACCACCCCCTCC
CTTGTCCCCACCATGCCCATGATGAGTCTTTTCTCAAGGCAGCTCGCCTTGCCTCCATCTCACCCTCACCTGTGCAC
CACAGCCACACTGGACATGGGTCCCTCTGAGCCTGAGTCCCTTCCCATTCCCACTGTCCCCTCTGGCAAGACCTTCC
TTCCAACACTGCCTTCATGCTCCTCCCTTGCCCCTGCAGGGCAGCCTCTCCCCTTGGCCCCTATTCCCTTAGGGGGC
TTGTGGCCACCCAGTCCTGGCACCTGACCTACAAGTTTGCCATCTTCATTCCCCCTTCTTCTGTTCATCAGCCCCCT
CCTCTATCCTCCCACCCTCACAGTTTTCCTTGTATATGAAATCTTCGTTCTTGTCCTTTTGCCCATGCGCATTTCCT
GCCTCCTCAGGGAGGTCGGGACAGCAGACCTGTGTGTTAAACATCAATGTGAAGTTATTTCCAGGAAGAAGTTTCAC
CTGTGATTTCCTCTTCCCCAGAGCCCCACAGTCTTCGTTACAACCTCATGGTGCTGTCCCAGGATGGATCTGTGCAG
TCAGGGTTTCTCGCTGAGGGACATCTGGATGGTCAGCCCTTCCTGCGCTATGACAGGCAGAAACGCAGGGCAAAGCC
CCAGGGACAGTGGGCAGAAAATGTCCTGGGAGCTAAGACCTGGGACACAGAGACCGAGGACTTGACAGAGAATGGGC
AAGACCTCAGGAGGACCCTGACTCATATCAAGGACCAGAAAGGAGGTGAGAGTCGGCAGGGGCAAGAGTAATGGGAG
GCCTTCTCCAGGAAAGTTGGAGACAGAGAGCAGGGACCTGTCTCTTCCCGCTGGATCTGGCTGGGGGTGGGGATGAG
GAATAGGGTCAGGGAGGCTCAGCAGGGTGGTGAGCCGGAACTCAGCCCACACAGGGAGGCATGGAGGAGGGCCAGGG
AGGGGTCGCTGCTGGGCTGAGTTCCTCACTTGGGTGGAAAGGTGATGGGTTCGGGAATGGAGAAGTCACTGCTGGGT
GGGGGCAGGCTTGCATTCCCTCCAGGAGATTAGGGTCTGTGAGATCCATGAAGACAGCAGCACCAGGGGCTCCCGGC
ATTTCTACTACGATGGGGAGCTCTTCCTCTCCCAAAACCTGGAGACTCAAGAATCGACAGTGCCCCAGTCCTCCAGA
GCTCAGACCTTGGCTATGAACGTCACAAATTTCTGGAAGGAAGATGCCATGAAGACCAAGACACACTATCGCGCTAT
GCAGGCAGACTGCCTGCAGAAACTACAGCGATATCTGAAATCCGGGGTGGCCATCAGGAGAACAGGTACCGACCCTG
GCCAGGGGCTCTACTGTTCCCGCAATTCTGCTAGAGTTGCCTCGCCTCCCAGCTCTGTCCGGGGAAACCCTCCCTGT
GCTATGGATGCAGGCGTTTCCTGTTGGCATATTGTGTCCTGATTTGCCTCTCCTGTTAGAGCCATTGGATAAAGACA
GTGGGTCTGGGACTGAACTGTCCAGTGTTGTAATCTGGGAAAGCAGTGGGCCCTCTGACAGAAGCCTGAGCCTGGTG
TGGGAGTTAGGCAGGAGAGGAAGCCCTCAGGGCCAGGGCTGCCCCCTCTGCCTCCCGGCCTGCCCATCCCGGAGAGT
TCCCTCCTGGCCCCATGACCCAGGAGTCCACCCTTGACATCCCCCTCCTCAGCATCAATGTGGGGATCCCAGAGCCT
GAGGCCACAGTCCCAAGGCCCATCCTCCTGCTAGCCTGGAGGAATTAGGCCCCAGGGTGAGGACAGACTTACAGAAG
GTCCGGTATCTGTGAGGGATTCAGCCAGAGTGAGAACAGTGGAGAGGAGCAGCCCTGTTCCCTGCATCTCCCTTAGA
GGGGAGCAGGGCTTCACTGGCTCTGCCCTTTCTTCTCCAGTGCCCCCCATGGTGAATGTCACCTGCAGCGAGGTCTC
AGAGGGCAACATCACCGTGACATGCAGGGCTTCCAGCTTCTATCCCCGGAATATCACACTGACCTGGCGTCAGGATG
GGGTATCTTTGAGCCACAACACCCAGCAGTGGGGGGATGTCCTGCCTGATGGGAATGGAACCTACCAGACCTGGGTG
GCCACCAGGATTCGCCAAGGAGAGGAGCAGAGGTTCACCTGCTACATGGAACACAGCGGGAATCACGGCACTCACCC
TGTGCCCTCTGGTGAGCCTGGGGTGACCCTGGAGAGGGTCAGGCCAGGGTAGGAACAGCAAGGACGGCTGTGGCTCT
CTGCCCAGTGTATAACAAGTCCCTTTTTTTCAGGGAAGGCGCTGGTGCTTCAGAGTCAACGGACAGACTTTCCATAT
GTTTCTGCTGCTATGCCATGTTTTGTTATTATTATTATTCTCTGTGTCCCTTGTTGCAAGAAGAAAACATCAGCGGC
AGAGGGTCCAGGTGAGAAAAGGGGACAGTTTCTGGAGATGGGAAAGCTCCTTTCTAGGCAGTAGGGTCTCCTCATTG
CTCCTGCCCAGACAAGACGTAGGTGACAAGGCTGCTGGGACAGGGGATGGAAGCTGGGGTATTTGGGAGGGGAATGG
GAGCTGCATCTCCATCTACACCCATAAGTGCTTCCCAAGCCAGGGCTGGGGCAAGGCCTTCGAATATCCAGCTGTGG
CCTCCTCCTGCTGCAAGTGAGGAGTGGGCAGCAGGGAGGGCTGTGGCACCTGCTCTGTCCCCATCCCAGCCTCTCTG
TCTCTCGGGCTCACTAGGGTGCGTCCAGGTGGGGTGAGTTGGGAATCACGTGCTGATTGCTGAGGGCCTGGATGATC
ATGGTGTCAGAGGGAGGAAATAGTAAAGGTGGCTGTGATCTGGGGAGGGCCAGAAACTGGAGAGGAATCCAAGGAGA
GGCGATGCCCACCCGTGTGCCTCCTCCAGGAGGCACTTTCCAGGTTCCCACTACCTGGCCTCCCTGAGTTTCCTTGC
AGATGACACAGATGAATAGATAAGCAGATGTCCCTGGGCCATTTGAGGAGCGGGGCCCAGCCCCTCATCAGGGCAGA
TGTGGTCCCTGTTTTCATCCTACCTCCAGCGTGTTTTCTTCTGCAGTCCCTGAGGGACACAGTCCCCAGGCGCCATC
TCTTTGAGGCTTTGTTCTGTGCTCTGTGGCCTTACCTTGCCCTCCCTGAGCCAATTTCCCTTTCTCAAGGTGGTCAC
TGCCTGGTAAGTTTGGAGTAAGGGACAGTCAGAAGCATTTCCCCCACAGTCAGGTTGTTTGATGGGAGATGAAAAGA
GACAGCAGAAGTTTTGTGTTTCTGCAAAAACAGAGGCAGTGCAGGGGACAGTGAGAGGCTGGGGTGTCCAGGAGACC
TGAGTCTGGCGGTAGGGGCGCTGGTTTCTCATCCTTGAACCTAGTTGCACTGTCAGTCGGCCCCTCATGCCTGAGCA
GATGGGAAGGTTCGTCCCCTGCCCTGCAGCAAGAGGGCCCCATCCAGGAGGCACCCACAGCAGGGGCAGTGCAGGTC
TGTGGTCACTCCTGCTCTCACCTGCGGCGTCTCCCGTGGAGGGATTGTCACTTCTGGTTCCCTGTGGGCAGGAATGG
TTTCCTCGTAGGTCACTGGGGTTTTGGCCAGGAAAAGGGTATGAAATTCATGTGCCAGTTTCTCAAAATTCCTGCTT
TCAATGTTGATGTCCAATAAAGATGTTCGTAATTTCAGCTCTATAATCTTAATAGGATTTCCTCTAATACTGCTGTT
GTAAAGCATATTAAATAAAACAGGAACTCAAATTTGGAGCCCCCTCTCCAGAAGGGTCTGTGTGGAGATGGTGGCTG
TGGCAGCGGCAGTTCCCAGGTGCAGAGGGTGGGCAGAGGCAGCCTCAGGCTAAGGGGTCTCCCCTACTCCACGTGGA
GAAAAGTCCTTGTAGGTTGCAAGGGCAGTGGCCTGGGTGGAATCCCTGCTAGGGACAGAGCAGGAAGGCCTCACAGC
CTCACCAAGCAGCAGCCCTGGGGTGAAGTAAGTGGACCAGGAGTAAGTGGACCAGGCAGGAGCAGTAGTGACTCAAC
AGCAGGTCACAGGCCTAGGTGGGTGCTGAAGGTCATGGGAGGCCAGGCCTCCTCGAGCAAGGTGGGGGGTCCCAGGG
TCAGGTCAGGTGCAGATCCTGTGGCAGCCACGTCTTTCCATGCTGGGCCTGCTGGGCCCCCCAGGCTTCCTGATGGG
GTCCCCAGTTAGGAGCTGCCTGCTCAGGGCTGGGAGGGGAGGAGTGCTGAGCTGCAGATAGAGGGCAGGGCCCACAG
TGGGCAGGGCCTGCCCTGGTGTGCAGGTGCCTCTGCAGGAGAGAAGGGCCTGGGGACTGAGAGCAAGGGTCAGGGCC
TCTCTTTGGGGAGGCCTCTCACTGTAACAGGACTGGTCAGGCCTGAGAGGAGGGCACTGGGTTCCCTCTTGGGTCTT
GTCCTTTTGTCTTGGGGCCCTTTCACTCCCTGCACGGTGAGTGGTGGGCACAGGACAGGGGCTGATGTTGATGGAGT
GATGGGAGAGAACTGACAGGGGCTGGGAAAAGCAAGGAGGGAGGAAGAAAAAAGTGGGGGCCTCATCTTCTCTCAGA
GAAAGGGCGAATCTGATTTTGGGGCAACTGAAGAGAGAAAAGTCCTTAGGGAATAAACACAACACTGCACCCAGTGG
AGCATTTACCCGTTTCCCTCTTCTCCAGAGCTTGTGAGCCTGCAGGTCCTGGATCAACACCCAGTTGGGACAGGAGA
CCACAGGGATGCAGCACAGCTGGGATTTCAGCCTCTGATGTCAGCTACTGGGTCCACTGGTTCCACTGAGGGCACCT
AGACTCTACAGCCAGGCGGCCAGGATTCAACTCCCTGCCTGGATCTCACCAGCACTTTCCCTCTGTTTCCTGACCTA
TGAAACAGAGAAAATAACATCACTTATTTATTGTTGTTGGATGCTGCAAAGTGTTAGTAGGTATGAGGTGTTTGCTG
CTCTGCCACGTAGAGAGCCAGCAAAGGGATCATGACCAACTCAACATTCCATTGGAGGCTATATGATCAAACAGCAA
ATTGTTTATCATGAATGCAGGATGTGGGCAAACTCACGACTGCTCCTGCCAACAGAAGGTTTGCTGAGGGCATTCAC
TCCATGGTGCTCATTGGAGTTATCTACTGGGTCATCTAGAGCCTATTGTTTGAGGAATGCAGTCTTACAAGCCTACT
CTGGACCCAGCAGCTGACTCCTTCTTCCACCCCTCTTCTTGCTATCTCCTATACCAATAAATACGAAGGGCTGTGGA
AGATCAGAGCCCTTGTTCACGAGAAGCAAGAAGCCCCCTGACCCCTTGTTCCAAATATACTCTTTTGTCTTTCTCTT
TATTCCCACGTTCGCCCTTTGTTCAGTCCAATACAGGGTTGTGGGGCCCTTAACAGTGCCATATTAATTGGTATCAT
TATTTCTGTTGTTTTTGTTTTTGTTTTTGTTTTTGTTTTTGAGACAGAGTCTCACTCTGTCACCCAGGCTGCAGTTC
ACTGGTGTGATCTCAGCTCACTGCAACCTCTGCCTCCCAGGTTCAAGCACTTCTCGTACCTCAGACTCCCGAATAGC
TGGGATTACAGACAGGCACCACCACACCCAGCTAATTTTTGTATTTTTTGTAGAGACGGGGTTTCGCCAAGTTGACC
AGCCCAGTTTCAAACTCCTGACCTCAGGTGATCTGCCTGCCTTGGCATCCCAAAGTGCTGGGATTACAAGAATGAGC
CACCGTGCCTGGCCTATTTTATTATATTGTAATATATTTTATTATATTAGCCACCATGCCTGTCCTATTTTCTTATG
TTTTAATATATTTTAATATATTACATGTGCAGTAATTAGATTATCATGGGTGAACTTTATGAGTGAGTATCTTGGTG
ATGACTCCTCCTGACCAGCCCAGGACCAGCTTTCTTGTCACCTTGAGGTCCCCTCGCCCCGTCACACCGTTATGCAT
TACTCTGTGTCTACTATTATGTGTGCATAATTTATACCGTAAATGTTTACTCTTTAAATAGA

| | |
|---|---|
| SEQ ID NO: 33 | MICB cDNA Sequence |

GAATTTTGTGAGCGACCGCGCTGGGCCGTTTCTCTTTCTTTTCCGGACCCTGCAGTGGCGCCTAAAGTCTGCGAGGA
GGAAGTCGCCTCTGTGCTCGTGAGTCCAGGGATCTAAGAGCCCCACAGTCTTCGTTACAACCTCATGGTGCTGTCCC
AGGATGGATCTGTGCAGTCAGGGTTTCTCGCTGAGGGACATCTGGATGGTCAGCCCTTCCTGCGCTATGACAGGCAG
AAACGCAGGGCAAAGCCCCAGGGACAGTGGGCAGAAAATGTCCTGGGAGCTAAGACCTGGGACACAGAGACCGAGGA
CTTGACAGAGAATGGGCAAGACCTCAGGAGGACCCTGACTCATATCAAGGACCAGAAAGGAGGCTTGCATTCCCTCC
AGGAGATTAGGGTCTGTGAGATCCATGAAGACAGCAGCACCAGGGGCTCCCGGCATTTCTACTACGATGGGGAGCTC
TTCCTCTCCCAAAACCTGGAGACTCAAGAATCGACAGTGCCCCAGTCCTCCAGAGCTCAGACCTTGGCTATGAACGT
CACAAATTTCTGGAAGGAAGATGCCATGAAGACCAAGACACACTATCGCGCTATGCAGGCAGACTGCCTGCAGAAAC
TACAGCGATATCTGAAATCCGGGGTGGCCATCAGGAGAACAGTGCCCCCCATGGTGAATGTCACCTGCAGCGAGGTC
TCAGAGGGCAACATCACCGTGACATGCAGGGCTTCCAGCTTCTATCCCCGGAATATCACACTGACCTGGCGTCAGGA
TGGGGTATCTTTGAGCCACAACACCCAGCAGTGGGGGGATGTCCTGCCTGATGGGAATGGAACCTACCAGACCTGGG
TGGCCACCAGGATTCGCCAAGGAGAGGAGCAGAGGTTCACCTGCTACATGGAACACAGCGGGAATCACGGCACTCAC
CCTGTGCCCTCTGGGAAGGCGCTGGTGCTTCAGAGTCAACGGACAGACTTTCCATATGTTTCTGCTGCTATGCCATG
TTTTGTTATTATTATTATTCTCTGTGTCCCTTGTTGCAAGAAGAAAACATCAGCGGCAGAGGGTCCAGAGCTTGTGA
GCCTGCAGGTCCTGGATCAACACCCAGTTGGGACAGGAGACCACAGGGATGCAGCACAGCTGGGATTTCAGCCTCTG
ATGTCAGCTACTGGGTCCACTGGTTCCACTGAGGGCACCTAGACTCTACAGCCAGGCGGCCAGGATTCAACTCCCTG
CCTGGATCTCACCAGCACTTTCCCTCTGTTTCCTGACCTATGAAACAGAGAAAATAACATCACTTATTTATTGTTGT
TGGATGCTGCAAAGTGTTAGTAGGTATGAGGTGTTTGCTGCTCTGCCACGTAGAGAGCCAGCAAAGGGATCATGACC
AACTCAACATTCCATTGGAGGCTATATGATCAAACAGCAAATTGTTTATCATGAATGCAGGATGTGGGCAAACTCAC
GACTGCTCCTGCCAACAGAAGGTTTGCTGAGGGCATTCACTCCATGGTGCTCATTGGAGTTATCTACTGGGTCATCT
AGAGCCTATTGTTTGAGGAATGCAGTCTTACAAGCCTACTCTGGACCCAGCAGCTGACTCCTTCTTCCACCCCTCTT
CTTGCTATCTCCTATACCAATAAATACGAAGGGCTGTGGAAGATCAGAGCCCTTGTTCACGAGAAGCAAGAAGCCCC
CTGACCCCTTGTTCCAAATATACTCTTTTGTCTTTCTCTTTATTCCCACGTTCGCCCTTTGTTCAGTCCAATACAGG
GTTGTGGGGCCCTTAACAGTGCCATATTAATTGGTATCATTATTTCTGTTGTTTTTGTTTTTGTTTTTGTTTTTGTT
TTTGAGACAGAGTCTCACTCTGTCACCCAGGCTGCAGTTCACTGGTGTGATCTCAGCTCACTGCAACCTCTGCCTCC
CAGGTTCAAGCACTTCTCGTACCTCAGACTCCCGAATAGCTGGGATTACAGACAGGCACCACCACACCCAGCTAATT
TTTGTATTTTTTGTAGAGACGGGGTTTCGCCAAGTTGACCAGCCCAGTTTCAAACTCCTGACCTCAGGTGATCTGCC
TGCCTTGGCATCCCAAAGTGCTGGGATTACAAGAATGAGCCACCGTGCCTGGCCTATTTTATTATATTGTAATATAT
TTTATTATATTAGCCACCATGCCTGTCCTATTTTCTTATGTTTTAATATATTTTAATATATTACATGTGCAGTAATT
AGATTATCATGGGTGAACTTTATGAGTGAGTATCTTGGTGATGACTCCTCCTGACCAGCCCAGGACCAGCTTTCTTG
TCACCTTGAGGTCCCCTCGCCCCGTCACACCGTTATGCATTACTCTGTGTCTACTATTATGTGTGCATAATTTATAC
CGTAAATGTTTACTCTTTAAATAGAAAAAAAAAAAAAAA

| | |
|---|---|
| SEQ ID NO: 34 | MICB Protein Sequence |

MVLSQDGSVQSGFLAEGHLDGQPFLRYDRQKRRAKPQGQWAENVLGAKTWDTETEDLTENGQDLRRTLTHIKDQKGG
LHSLQEIRVCEIHEDSSTRGSRHFYYDGELFLSQNLETQESTVPQSSRAQTLAMNVTNFWKEDAMKTKTHYRAMQAD
CLQKLQRYLKSGVAIRRTVPPMVNVTCSEVSEGNITVTCRASSFYPRNITLTWRQDGVSLSHNTQQWGDVLPDGNGT
YQTWVATRIRQGEEQRFTCYMEHSGNHGTHPVPSGKALVLQSQRTDFPYVSAAMPCFVIIIILCVPCCKKKTSAAEG
PELVSLQVLDQHPVGTGDHRDAAQLGFQPLMSATGSTGSTEGT

| | |
|---|---|
| SEQ ID NO: 37 | CD46 cDNA Sequence |

GGAACTCGGAGAGGTCTCCGCTAGGCTGGTGTCGGGTTACCTGCTCATCTTCCCGAAAATGATGGCGTTTTGCGCGC
TGCGCAAGGCACTTCCCTGCCGTCCCGAGAATCCCTTTTCTTCGAGGTGCTTCGTTGAGATTCTTTGGGTGTCGTTG
GCCCTAGTGTTCCTGCTTCCCATGCCCTCAGATGCCTGTGATGAGCCACCGAAGTTTGAAAGCATGCGGCCCCAATT
TTTGAATACCACTTACAGACCTGGAGACCGTGTAGAGTATGAATGTCGCCCCGGGTTCCAGCCCATGGTTCCTGCGC
TTCCCACCTTTTCCGTCTGTCAGGACGATAATACGTGGTCACCCCTCCAGGAGGCTTGTCGACGAAAAGCCTGTTCG
AATCTACCAGACCCGTTAAATGGCCAAGTTAGCTACCCAAATGGGGATATGCTGTTTGGTTCAAAGGCTCAGTTTAC
CTGTAACACTGGTTTTTACATAATTGGAGCCGAGACTGTGTATTGTCAGGTTTCTGGGAATGTTATGGCCTGGAGTG
AGCCCTCCCCGCTATGTGAGAAGATTTTGTGTAAACCACCTGGCGAAATTCCAAATGGAAAATACACCAATAGCCAT
AAGGATGTATTTGAATACAATGAAGTAGTAACTTACAGTTGTCTTTCTTCAACTGGACCGGATGAATTTTCACTTGT
TGGAGAGAGCAGCCTTTTTTGTATTGGGAAGGACGAGTGGAGTAGTGACCCCCCTGAGTGTAAAGTGGTCAAATGTC
CATATCCAGTAGTCCCAAATGGAGAAATTGTATCAGGATTTGGATCAAAATTTTACTACAAAGCAGAGGTTGTATTT
AAATGCAATGCTGGTTTTACCCTTCATGGCAGAGACACAATTGTCTGCGGTGCAAACAGCACGTGGGAGCCTGAGAT
GCCCCAATGTATCAAAGATTCCAAGCCTACTGATCCACCTGCAACCCCAGGACCAAGCCATCCAGGACCTCCCAGTC
CCAGTGATGCATCACCACCTAAAGATGCTGAGAGTTTAGATGGAGGAATCATCGCTGCAATTGTTGTGGGCGTCTTA
GCTGCCATTGCAGTAATTGCTGGTGGTGTATACTTTTTTCATCATAAATACAACAAGAAAAGGTCGAAGTAAAACTG
ATGTGCTTAAAGTAAAAGTTGCTGAGAGGACGTGGAATCCAGCCCCTTCCCTCTCCTGTGCTGCTGCCTGGGTCCCG
TTTTGCATGTCATGACTGTGTGCTTCCAAAAAATGCCTTTTGTTCGTATTTTTTTGCCTAAACGCATGATTTTGTCT
CTACTTGAATTAAATCATCACTGAATCCACGC

| | |
|---|---|
| SEQ ID NO: 38 | CD55 cDNA Sequence |

CGGCACGAGATTTCGTCTTAATCGCGGAGGTCGCAGAGTCCGGGAGCCGCTCGGGGTCCCCGTTCCCGCGCGCCATG
AGTCCCCTGCCGCGGAGCGCCCCCGCGGTGAGGCGCCTAATGGGCGGACAGACGCCGCCGCCGCTGCTGCTGCTGCT
GCTGCTGCTGTGTATCCCGGCTGCGCAGGGTGACTGCAGCCTTCCACCCGATGTACCTAATGCCCAACCAGATTTGC
GAGGTCTTGCAAGTTTTCCTGAACAAACCACAATAACATACAAATGTAACAAAGGCTTTGTCAAAGTTCCTGGCATG
GCAGACTCAGTGCTCTGTCTTAATGATAAATGGTCAGAAGTTGCAGAATTTTGTAATCGTAGCTGTGATGTTCCAAC
CAGGCTACATTTTGCATCTCTTAAAAAGTCTTACAGCAAACAGAATTATTTCCCAGAGGGTTTCACCGTGGAATATG
AGTGCCGTAAGGGCTATAAAAGGGATCTTACTCTATCAGAAAAACTAACTTGCCTTCAGAATTTTACGTGGTCCAAA
CCTGATGAATTTTGCAAAAAAAAACAATGTCCGACTCCTGGAGAACTAAAAAATGGTCATGTCAATATAACAACTGA
CTTGTTATTTGGCGCATCCATCTTTTTCTCATGTAACGCAGGGTACAGACTAGTTGGTGCAACTTCTAGTTACTGTT
TTGCCATAGCAAATGATGTTGAGTGGAGTGATCCATTGCCAGAATGCCAAGAAATTTCTCCAACTGTCAAAGCCATA
CCAGCTGTTGAGAAACCCATCACAGTAAATTTTCCAGCAACAAAGTATCCAGCTATTCCCAGGGCCACAACGAGTTT
TCATTCAAGTACATCTAAAAATCGAGGAAACCCTTCTTCAGGCATGAGAATCATGTCGTCTGGTACCATGCTACTTA
TTGCAGGAGGTGTTGCTGTTATTATAATAATTGTTGCCCTAATTCTAGCCAAAGGTTTCTGGCACTATGGAAAATCA
GGCTCTTACCACACTCATGAGAACAACAAAGCCGTTAATGTTGCATTTTATAATTTACCTGCGACTGGCGATGCCGC
AGATGTAAGACCTGGTAATTAACAAAAGGACGGTGCATGTGTAACACTGACAGTTTTGCTTATGGTGCTAGTAACCA
TTGGCTAGCTGACTTAGCCAAAGAAGAGTTAAGAAGAAAGTGCACACAAGTACACAGAATATTTTCAGTTTCTTAGA
ACTTTCAGGTGGAGTGGACATAGTTTGTGGATAGTGTTCTTCGTTTTGCATGTTTTCATTGTCTCTAAGGTACATAG
GAATGTCACAGAACCAAAGAGAAACAAATCTATCCTGAAATTACATCCTCAACACTCCTAAGACTCTTGAAAATAGA
ACAGCTCATAAGATTGAGAGCAATTACTTTCCAAAAAGGGTGAGAAAATGGAGAGATTTGTTCATGGTTAGAATATA
AGAAAAAAGAAAACAAAAAGGTGATTTTTCCCACCAAATGTGTAATGTTATTTTTATTAATAAAGGAAAAAAAAAAA
AAAAAA

| | |
|---|---|
| SEQ ID NO: 39 | CD59 cDNA Sequence |

GAAAAGACGCGCAGGCCGGGCCGCTCTCCCGACGGGGAGTAGCGCTGCAGCCGGACGCAGGGTGCAGTTAGAATCCA
TAGACGGTCACGATGGGAAGCAAAGGAGGGTTCATTTTGCTCTGGCTCCTGTCCATCCTGGCTGTTCTCTGCCACTT
AGGTCACAGCCTGCAGTGCTATAACTGTATCAACCCAGCTGGTAGCTGCACTACGGCCATGAATTGTTCACATAATC
AGGATGCCTGTATCTTCGTTGAAGCCGTGCCACCCAAAACTTACTACCAGTGTTGGAGGTTCGATGAATGCAATTTC
GATTTCATTTCGAGAAACCTAGCGGAGAAGAAGCTGAAGTACAACTGCTGCCGGAAGGACCTGTGTAACAAGAGTGA
TGCCACGATTTCATCAGGGAAAACCGCTCTGCTGGTGATCCTGCTGCTGGTAGCAACCTGGCACTTTTGTCTCTAAC
TGTACACCAGGAGAGTTTCTCCTCAACTTCCTCTGTCTCTCTGTTCCTATTTCCCATGCTGCGGTGTTCCAAAGGCT
GTGTATGCTCCAGCTTCTTCCTGTTGGGAAGGACTAAACCTAGCTTGAGCACTTTGGATTAGAGAGAGAAACTTTGA
GCGACTTTGAAGACCAGGCCTGTTGGCAGAGAAGACCTGTCAGAGGGGAAACGTTTTAAGAGTGAAGCACAGGTGAT
TTGAGCGAGGCCTATGCGTCTTCCTCTGCTCTTGGCAGGACCAGCTTTGCGGTAACCATTCGATAGATTCCACAATC
CTT

| | |
|---|---|
| SEQ ID NO: 40 | ICP47 cDNA Sequence |

TCAAGGGGCCAGCACGCGATCCTGCCGCTCGTTCGATCTAGCACACCCACGGGTCTGCTGTGTGGGATTTCGACTCG
CGGGATCCGATCGCACGTCCGGAGGACACAGCAGCGGGAGCTCCGGGTCGGTCACCGCAGTTCTGGCCGCCTCTCGG
TCCTCCCGTTCCCTTTTATGGATCTCCGCGCAGACATCGCCATACGTCCGGTGTGTGCACCGCGAAGAATCCAGAAA
CATGTCCGTCGTTTTCAGGGCCCAAGACAT

| | |
|---|---|
| SEQ ID NO: 41 | HLA-G1 cDNA Sequence |

AGTGTGGTACTTTGTCTTGAGGAGATGTCCTGGACTCACACGGAAACTTAGGGCTACGGAATGAAGTTCTCACTCCC
ATTAGGTGACAGGTTTTTAGAGAAGCCAATCAGCGTCGCCGCGGTCCTGGTTCTAAAGTCCTCGCTCACCCACCCGG
ACTCATTCTCCCCAGACGCCAAGGATGGTGGTCATGGCGCCCCGAACCCTCTTCCTGCTGCTCTCGGGGGCCCTGAC
CCTGACCGAGACCTGGGCGGGCTCCCACTCCATGAGGTATTTCAGCGCCGCCGTGTCCCGGCCCGGCCGCGGGGAGC
CCCGCTTCATCGCCATGGGCTACGTGGACGACACGCAGTTCGTGCGGTTCGACAGCGACTCGGCGTGTCCGAGGATG
GAGCCGCGGGCGCCGTGGGTGGAGCAGGAGGGGCCGGAGTATTGGGAAGAGGAGACACGGAACACCAAGGCCCACGC
ACAGACTGACAGAATGAACCTGCAGACCCTGCGCGGCTACTACAACCAGAGCGAGGCCAGTTCTCACACCCTCCAGT
GGATGATTGGCTGCGACCTGGGGTCCGACGGACGCCTCCTCCGCGGGTATGAACAGTATGCCTACGATGGCAAGGAT
TACCTCGCCCTGAACGAGGACCTGCGCTCCTGGACCGCAGCGGACACTGCGGCTCAGATCTCCAAGCGCAAGTGTGA
GGCGGCCAATGTGGCTGAACAAAGGAGAGCCTACCTGGAGGGCACGTGCGTGGAGTGGCTCCACAGATACCTGGAGA
ACGGGAAGGAGATGCTGCAGCGCGCGGACCCCCCCAAGACACACGTGACCCACCACCCTGTCTTTGACTATGAGGCC
ACCCTGAGGTGCTGGGCCCTGGGCTTCTACCCTGCGGAGATCATACTGACCTGGCAGCGGGATGGGGAGGACCAGAC
CCAGGACGTGGAGCTCGTGGAGACCAGGCCTGCAGGGGATGGAACCTTCCAGAAGTGGGCAGCTGTGGTGGTGCCTT
CTGGAGAGGAGCAGAGATACACGTGCCATGTGCAGCATGAGGGGCTGCCGGAGCCCCTCATGCTGAGATGGAAGCAG
TCTTCCCTGCCCACCATCCCCATCATGGGTATCGTTGCTGGCCTGGTTGTCCTTGCAGCTGTAGTCACTGGAGCTGC
GGTCGCTGCTGTGCTGTGGAGAAAGAAGAGCTCAGATTGAAAAGGAGGGAGCTACTCTCAGGCTGCAATGTGAAACA
GCTGCCCTGTGTGGGACTGAGTGGCAAGTCCCTTTGTGACTTCAAGAACCCTGACTCCTCTTTGTGCAGAGACCAGC
CCACCCCTGTGCCCACCATGACCCTCTTCCTCATGCTGAACTGCATTCCTTCCCCAATCACCTTTCCTGTTCCAGAA
AAGGGGCTGGGATGTCTCCGTCTCTGTCTCAAATTTGTGGTCCACTGAGCTATAACTTACTTCTGTATTAAAATTAG
AATCTGAGTATAAATTTACTTTTTCAAATTATTTCCAAGAGAGATTGATGGGTTAATTAAAGGAGAAGATTCCTGAA
ATTTGAGAGACAAAATAAATGGAAGACATGAGAACTTT

| | |
|---|---|
| SEQ ID NO: 42 | HLA-E cDNA Sequence |

GCAGACTCAGTTCTCATTCCCAATGGGTGTCGGGTTTCTAGAGAAGCCAATCAGCGTCGCCACGACTCCCGACTATA
AAGTCCCCATCCGGACTCAAGAAGTTCTCAGGACTCAGAGGCTGGGATCATGGTAGATGGAACCCTCCTTTTACTCC
TCTCGGAGGCCCTGGCCCTTACCCAGACCTGGGCGGGCTCCCACTCCTTGAAGTATTTCCACACTTCCGTGTCCCGG
CCCGGCCGCGGGGAGCCCCGCTTCATCTCTGTGGGCTACGTGGACGACACCCAGTTCGTGCGCTTCGACAACGACGC
CGCGAGTCCGAGGATGGTGCCGCGGGCGCCGTGGATGGAGCAGGAGGGGTCAGAGTATTGGGACCGGGAGACACGGA
GCGCCAGGGACACCGCACAGATTTTCCGAGTGAATCTGCGGACGCTGCGCGGCTACTACAATCAGAGCGAGGCCGGG
TCTCACACCCTGCAGTGGATGCATGGCTGCGAGCTGGGGCCCGACGGGCGCTTCCTCCGCGGGTATGAACAGTTCGC
CTACGACGGCAAGGATTATCTCACCCTGAATGAGGACCTGCGCTCCTGGACCGCGGTGGACACGGCGGCTCAGATCT
CCGAGCAAAAGTCAAATGATGCCTCTGAGGCGGAGCACCAGAGAGCCTACCTGGAAGACACATGCGTGGAGTGGCTC
CACAAATACCTGGAGAAGGGGAAGGAGACGCTGCTTCACCTGGAGCCCCCAAAGACACACGTGACTCACCACCCCAT
CTCTGACCATGAGGCCACCCTGAGGTGCTGGGCCCTGGGCTTCTACCCTGCGGAGATCACACTGACCTGGCAGCAGG
ATGGGGAGGGCCATACCCAGGACACGGAGCTCGTGGAGACCAGGCCTGCAGGGGATGGAACCTTCCAGAAGTGGGCA
GCTGTGGTGGTGCCTTCTGGAGAGGAGCAGAGATACACGTGCCATGTGCAGCATGAGGGGCTACCCGAGCCCGTCAC
CCTGAGATGGAAGCCGGCTTCCCAGCCCACCATCCCCATCGTGGGCATCATTGCTGGCCTGGTTCTCCTTGGATCTG
TGGTCTCTGGAGCTGTGGTTGCTGCTGTGATATGGAGGAAGAAGAGCTCAGGTGGAAAAGGAGGGAGCTACTCTAAG
GCTGAGTGGAGCGACAGTGCCCAGGGGTCTGAGTCTCACAGCTTGTAAAGCCTGAGACAGCTGCCTTGTGTGCGACT
GAGATGCACAGCTGCCTTGTGTGCGACTGAGATGCAGGATTTCCTCACGCCTCCCCTATGTGTCTTAGGGGACTCTG
GCTTCTCTTTTTGCAAGGGCCTCTGAATCTGTCTGTGTCCCTGTTAGCACAATGTGAGGAGGTAGAGAAACAGTCCA
CCTCTGTGTCTACCATGACCCCCTTCCTCACACTGACCTGTGTTCCTTCCCTGTTCTCTTTTCTATTAAAAATAAGA
ACCTGGGCAGAGTGCGGCAGCTCATGCCTGTAATCCCAGCACTTAGGGAGGCCGAGGAGGGCAGATCACGAGGTCAG
GAGATCGAAACCATCCTGGCTAACACGGTGAAACCCCGTCTCTACTAAAAAATACAAAAAATTAGCTGGGCGCAGAG
GCACGGGCCTGTAGTCCCAGCTACTCAGGAGGCGGAGGCAGGAGAATGGCGTCAACCCGGGAGGCGGAGGTTGCAGT
GAGCCAGGATTGTGCGACTGCACTCCAGCCTGGGTGACAGGGTGAAACGCCATCTCAAAAAATAAAAATTGAAAAAT
AAAAAAAGAACCTGGATCTCAATTTAATTTTTCATATTCTTGCAATGAAATGGACTTGAGGAAGCTAAGATCATAGC
TAGAAATACAGATAATTCCACAGCACATCTCTAGCAAATTTAGCCTATTCCTATTCTCTAGCCTATTCCTTACCACC
TGTAATCTTGACCATATACCTTGGAGTTGAATATTGTTTTCATACTGCTGTGGTTTGAATGTTCCCTCCAACACTCA
TGTTGAGACTTAATCCCTAATGTGGCAATACTGAAAGGTGGGGCCTTTGAGATGTGATTGGATCGTAAGGCTGTGCC
TTCATTCATGGGTTAATGGATTAATGGGTTATCACAGGAATGGGACTGGTGGCTTTATAAGAAGAGGAAAAGAGAAC
TGAGCTAGCATGCCCAGCCCACAGAGAGCCTCCACTAGAGTGATGCTAAGTGGAAATGTGAGGTGCAGCTGCCACAG
AGGGCCCCCACCAGGGAAATGTCTAGTGTCTAGTGGATCCAGGCCACAGGAGAGAGTGCCTTGTGGAGCGCTGGGAG
CAGGACCTGACCACCACCAGGACCCCAGAACTGTGGAGTCAGTGGCAGCATGCAGCGCCCCCTTGGGAAAGCTTTAG
GCACCAGCCTGCAACCCATTCGAGCAGCCACGTAGGCTGCACCCAGCAAAGCCACAGGCACGGGGCTACCTGAGGCC
TTGGGGGCCCAATCCCTGCTCCAGTGTGTCCGTGAGGCAGCACACGAAGTCAAAAGAGATTATTCTCTTCCCACAGA
TACCTTTTCTCTCCCATGACCCTTTAACAGCATCTGCTTCATTCCCCTCACCTTCCCAGGCTGATCTGAGGTAAACT
TTGAAGTAAAATAAAAGCTGTGTTTGAGCATCATTTGTATTTCAAAAAAAAAAAAAAAAAAAA

| | |
|---|---|
| SEQ ID NO: 43 | Human β-2-microglobulin cDNA Sequence |

AATATAAGTGGAGGCGTCGCGCTGGCGGGCATTCCTGAAGCTGACAGCATTCGGGCCGAGATGTCTCGCTCCGTGGC
CTTAGCTGTGCTCGCGCTACTCTCTCTTTCTGGCCTGGAGGCTATCCAGCGTACTCCAAAGATTCAGGTTTACTCAC
GTCATCCAGCAGAGAATGGAAAGTCAAATTTCCTGAATTGCTATGTGTCTGGGTTTCATCCATCCGACATTGAAGTT
GACTTACTGAAGAATGGAGAGAGAATTGAAAAAGTGGAGCATTCAGACTTGTCTTTCAGCAAGGACTGGTCTTTCTA
TCTCTTGTACTACACTGAATTCACCCCCACTGAAAAAGATGAGTATGCCTGCCGTGTGAACCATGTGACTTTGTCAC
AGCCCAAGATAGTTAAGTGGGATCGAGACATGTAAGCAGCATCATGGAGGTTTGAAGATGCCGCATTTGGATTGGAT
GAATTCCAAATTCTGCTTGCTTGCTTTTTAATATTGATATGCTTATACACTTACACTTTATGCACAAAATGTAGGGT
TATAATAATGTTAACATGGACATGATCTTCTTTATAATTCTACTTTGAGTGCTGTCTCCATGTTTGATGTATCTGAG
CAGGTTGCTCCACAGGTAGCTCTAGGAGGGCTGGCAACTTAGAGGTGGGGAGCAGAGAATTCTCTTATCCAACATCA
ACATCTTGGTCAGATTTGAACTCTTCAATCTCTTGCACTCAAAGCTTGTTAAGATAGTTAAGCGTGCATAAGTTAAC
TTCCAATTTACATACTCTGCTTAGAATTTGGGGGAAAATTTAGAAATATAATTGACAGGATTATTGGAAATTTGTTA
TAATGAATGAAACATTTTGTCATATAAGATTCATATTTACTTCTTATACATTTGATAAAGTAAGGCATGGTTGTGGT
TAATCTGGTTTATTTTTGTTCCACAAGTTAAATAAATCATAAAACTTGATGTGTTATCTCTTA

| | |
|---|---|
| SEQ ID NO: 44 | Human PD-L1 cDNA Sequence |

GGCGCAACGCTGAGCAGCTGGCGCGTCCCGCGCGGCCCCAGTTCTGCGCAGCTTCCCGAGGCTCCGCACCAGCCGCG
CTTCTGTCCGCCTGCAGGGCATTCCAGAAAGATGAGGATATTTGCTGTCTTTATATTCATGACCTACTGGCATTTGC
TGAACGCCCCATACAACAAAATCAACCAAAGAATTTTGGTTGTGGATCCAGTCACCTCTGAACATGAACTGACATGT
CAGGCTGAGGGCTACCCCAAGGCCGAAGTCATCTGGACAAGCAGTGACCATCAAGTCCTGAGTGGTAAGACCACCAC
CACCAATTCCAAGAGAGAGGAGAAGCTTTTCAATGTGACCAGCACACTGAGAATCAACACAACAACTAATGAGATTT
TCTACTGCACTTTTAGGAGATTAGATCCTGAGGAAAACCATACAGCTGAATTGGTCATCCCAGAACTACCTCTGGCA
CATCCTCCAAATGAAAGGACTCACTTGGTAATTCTGGGAGCCATCTTATTATGCCTTGGTGTAGCACTGACATTCAT
CTTCCGTTTAAGAAAAGGGAGAATGATGGATGTGAAAAAATGTGGCATCCAAGATACAAACTCAAAGAAGCAAAGTG
ATACACATTTGGAGGAGACGTAATCCAGCATTGGAACTTCTGATCTTCAAGCAGGGATTCTCAACCTGTGGTTTAGG
GGTTCATCGGGGCTGAGCGTGACAAGAGGAAGGAATGGGCCCGTGGGATGCAGGCAATGTGGGACTTAAAAGGCCCA
AGCACTGAAAATGGAACCTGGCGAAAGCAGAGGAGGAGAATGAAGAAAGATGGAGTCAAACAGGGAGCCTGGAGGGA
GACCTTGATACTTTCAAATGCCTGAGGGGCTCATCGACGCCTGTGACAGGGAGAAAGGATACTTCTGAACAAGGAGC
CTCCAAGCAAATCATCCATTGCTCATCCTAGGAAGACGGGTTGAGAATCCCTAATTTGAGGGTCAGTTCCTGCAGAA
GTGCCCTTTGCCTCCACTCAATGCCTCAATTTGTTTTCTGCATGACTGAGAGTCTCAGTGTTGGAACGGGACAGTAT
TTATGTATGAGTTTTTCCTATTTATTTTGAGTCTGTGAGGTCTTCTTGTCATGTGAGTGTGGTTGTGAATGATTTCT
TTTGAAGATATATTGTAGTAGATGTTACAATTTTGTCGCCAAACTAAACTTGCTGCTTAATGATTTGCTCACATCTA
GTAAAACATGGAGTATTTGTAAGGTGCTTGGTCTCCTCTATAACTACAAGTATACATTGGAAGCATAAAGATCAAAC
CGTTGGTTGCATAGGATGTCACCTTTATTTAACCCATTAATACTCTGGTTGACCTAATCTTATTCTCAGACCTCAAG
TGTCTGTGCAGTATCTGTTCCATTTAAATATCAGCTTTACAATTATGTGGTAGCCTACACACATAATCTCATTTCAT
CGCTGTAACCACCCTGTTGTGATAACCACTATTATTTTACCCATCGTACAGCTGAGGAAGCAAACAGATTAAGTAAC
TTGCCCAAACCAGTAAATAGCAGACCTCAGACTGCCACCCACTGTCCTTTTATAATACAATTTACAGCTATATTTTA
CTTTAAGCAATTCTTTTATTCAAAAACCATTTATTAAGTGCCCTTGCAATATCAATCGCTGTGCCAGGCATTGAATC
TACAGATGTGAGCAAGACAAAGTACCTGTCCTCAAGGAGCTCATAGTATAATGAGGAGATTAACAAGAAAATGTATT
ATTACAATTTAGTCCAGTGTCATAGCATAAGGATGATGCGAGGGGAAAACCCGAGCAGTGTTGCCAAGAGGAGGAAA
TAGGCCAATGTGGTCTGGGACGGTTGGATATACTTAAACATCTTAATAATCAGAGTAATTTTCATTTACAAAGAGAG
GTCGGTACTTAAAATAACCCTGAAAAATAACACTGGAATTCCTTTTCTAGCATTATATTTATTCCTGATTTGCCTTT
GCCATATAATCTAATGCTTGTTTATATAGTGTCTGGTATTGTTTAACAGTTCTGTCTTTTCTATTTAAATGCCACTA
AATTTTAAATTCATACCTTTCCATGATTCAAAATTCAAAAGATCCCATGGGAGATGGTTGGAAAATCTCCACTTCAT
CCTCCAAGCCATTCAAGTTTCCTTTCCAGAAGCAACTGCTACTGCCTTTCATTCATATGTTCTTCTAAAGATAGTCT
ACATTTGGAAATGTATGTTAAAAGCACGTATTTTTAAAATTTTTTTCCTAAATAGTAACACATTGTATGTCTGCTGT
GTACTTTGCTATTTTTATTTATTTTAGTGTTTCTTATATAGCAGATGGAATGAATTTGAAGTTCCCAGGGCTGAGGA
TCCATGCCTTCTTTGTTTCTAAGTTATCTTTCCCATAGCTTTTCATTATCTTTCATATGATCCAGTATATGTTAAAT
ATGTCCTACATATACATTTAGACAACCACCATTTGTTAAGTATTTGCTCTAGGACAGAGTTTGGATTTGTTTATGTT
TGCTCAAAAGGAGACCCATGGGCTCTCCAGGGTGCACTGAGTCAATCTAGTCCTAAAAAGCAATCTTATTATTAACT
CTGTATGACAGAATCATGTCTGGAACTTTTGTTTTCTGCTTTCTGTCAAGTATAAACTTCACTTTGATGCTGTACTT
GCAAAATCACATTTTCTTTCTGGAAATTCCGGCAGTGTACCTTGACTGCTAGCTACCCTGTGCCAGAAAAGCCTCAT
TCGTTGTGCTTGAACCCTTGAATGCCACCAGCTGTCATCACTACACAGCCCTCCTAAGAGGCTTCCTGGAGGTTTCG
AGATTCAGATGCCCTGGGAGATCCCAGAGTTTCCTTTCCCTCTTGGCCATATTCTGGTGTCAATGACAAGGAGTACC
TTGGCTTTGCCACATGTCAAGGCTGAAGAAACAGTGTCTCCAACAGAGCTCCTTGTGTTATCTGTTTGTACATGTGC
ATTTGTACAGTAATTGGTGTGACAGTGTTCTTTGTGTGAATTACAGGCAAGAATTGTGGCTGAGCAAGGCACATAGT
CTACTCAGTCTATTCCTAAGTCCTAACTCCTCCTTGTGGTGTTGGATTTGTAAGGCACTTTATCCCTTTTGTCTCAT
GTTTCATCGTAAATGGCATAGGCAGAGATGATACCTAATTCTGCATTTGATTGTCACTTTTTGTACCTGCATTAATT
TAATAAAATATTCTTATTTATTTTGTTACTTGGTACACCAGCATGTCCATTTTCTTGTTTATTTTGTGTTTAATAAA
ATGTTCAGTTTAACATCCCAGTGGAGAAAGTTAAAAAA

| | |
|---|---|
| SEQ ID NO: 45 | Human PD-L2 cDNA Sequence |

GCAAACCTTAAGCTGAATGAACAACTTTTCTTCTCTTGAATATATCTTAACGCCAAATTTTGAGTGCTTTTTTGTTA
CCCATCCTCATATGTCCCAGCTAGAAAGAATCCTGGGTTGGAGCTACTGCATGTTGATTGTTTTGTTTTTCCTTTTG
GCTGTTCATTTTGGTGGCTACTATAAGGAAATCTAACACAAACAGCAACTGTTTTTTGTTGTTTACTTTTGCATCTT
TACTTGTGGAGCTGTGGCAAGTCCTCATATCAAATACAGAACATGATCTTCCTCCTGCTAATGTTGAGCCTGGAATT
GCAGCTTCACCAGATAGCAGCTTTATTCACAGTGACAGTCCCTAAGGAACTGTACATAATAGAGCATGGCAGCAATG
TGACCCTGGAATGCAACTTTGACACTGGAAGTCATGTGAACCTTGGAGCAATAACAGCCAGTTTGCAAAAGGTGGAA
AATGATACATCCCCACACCGTGAAAGAGCCACTTTGCTGGAGGAGCAGCTGCCCCTAGGGAAGGCCTCGTTCCACAT
ACCTCAAGTCCAAGTGAGGGACGAAGGACAGTACCAATGCATAATCATCTATGGGGTCGCCTGGGACTACAAGTACC
TGACTCTGAAAGTCAAAGCTTCCTACAGGAAAATAAACACTCACATCCTAAAGGTTCCAGAAACAGATGAGGTAGAG
CTCACCTGCCAGGCTACAGGTTATCCTCTGGCAGAAGTATCCTGGCCAAACGTCAGCGTTCCTGCCAACACCAGCCA
CTCCAGGACCCCTGAAGGCCTCTACCAGGTCACCAGTGTTCTGCGCCTAAAGCCACCCCCTGGCAGAAACTTCAGCT
GTGTGTTCTGGAATACTCACGTGAGGGAACTTACTTTGGCCAGCATTGACCTTCAAAGTCAGATGGAACCCAGGACC
CATCCAACTTGGCTGCTTCACATTTTCATCCCCTTCTGCATCATTGCTTTCATTTTCATAGCCACAGTGATAGCCCT
AAGAAAACAACTCTGTCAAAAGCTGTATTCTTCAAAAGACACAACAAAAAGACCTGTCACCACAACAAAGAGGGAAG
TGAACAGTGCTATCTGAACCTGTGGTCTTGGGAGCCAGGGTGACCTGATATGACATCTAAAGAAGCTTCTGGACTCT
GAACAAGAATTCGGTGGCCTGCAGAGCTTGCCATTTGCACTTTTCAAATGCCTTTGGATGACCCAGCACTTTAATCT
GAAACCTGCAACAAGACTAGCCAACACCTGGCCATGAAACTTGCCCCTTCACTGATCTGGACTCACCTCTGGAGCCT
ATGGCTTTAAGCAAGCACTACTGCACTTTACAGAATTACCCCACTGGATCCTGGACCCACAGAATTCCTTCAGGATC
CTTCTTGCTGCCAGACTGAAAGCAAAAGGAATTATTTCCCCTCAAGTTTTCTAAGTGATTTCCAAAAGCAGAGGTGT
GTGGAAATTTCCAGTAACAGAAACAGATGGGTTGCCAATAGAGTTATTTTTTATCTATAGCTTCCTCTGGGTACTAG
AAGAGGCTATTGAGACTATGAGCTCACAGACAGGGCTTCGCACAAACTCAAATCATAATTGACATGTTTTATGGATT
ACTGGAATCTTGATAGCATAATGAAGTTGTTCTAATTAACAGAGAGCATTTAAATATACACTAAGTGCACAAATTGT
GGAGTAAAGTCATCAAGCTCTGTTTTTGAGGTCTAAGTCACAAAGCATTTGTTTTAACCTGTAATGGCACCATGTTT
AATGGTGGTTTTTTTTTTGAACTACATCTTTCCTTTAAAAATTATTGGTTTCTTTTTATTTGTTTTTACCTTAGAAA
TCAATTATATACAGTCAAAAATATTTGATATGCTCATACGTTGTATCTGCAGCAATTTCAGATAAGTAGCTAAAATG
GCCAAAGCCCCAAACTAAGCCTCCTTTTCTGGCCCTCAATATGACTTTAAATTTGACTTTTCAGTGCCTCAGTTTGC
ACATCTGTAATACAGCAATGCTAAGTAGTCAAGGCCTTTGATAATTGGCACTATGGAAATCCTGCAAGATCCCACTA
CATATGTGTGGAGCAGAAGGGTAACTCGGCTACAGTAACAGCTTAATTTTGTTAAATTTGTTCTTTATACTGGAGCC
ATGAAGCTCAGAGCATTAGCTGACCCTTGAACTATTCAAATGGGCACATTAGCTAGTATAACAGACTTACATAGGTG
GGCCTAAAGCAAGCTCCTTAACTGAGCAAAATTTGGGGCTTATGAGAATGAAAGGGTGTGAAATTGACTAACAGACA
AATCATACATCTCAGTTTCTCAATTCTCATGTAAATCAGAGAATGCCTTTAAAGAATAAAACTCAATTGTTATTCTT
CAACGTTCTTTATATATTCTACTTTTGGGTA

| | |
|---|---|
| SEQ ID NO: 46 | Human Spi9 cDNA Sequence |

AGCGGGAGTCCGCGGCGAGCGCAGCAGCAGGGCCGGGTCCTGCGCCTCGGGGGTCGGCGTCCAGGCTCGGAGCGCGG
CACGGAGACGGCGGCAGCGCTGGACTAGGTGGCAGGCCCTGCATCATGGAAACTCTTTCTAATGCAAGTGGTACTTT
TGCCATACGCCTTTTAAAGATACTGTGTCAAGATAACCCTTCGCACAACGTGTTCTGTTCTCCTGTGAGCATCTCCT
CTGCCCTGGCCATGGTTCTCCTAGGGGCAAAGGGAAACACCGCAACCCAGATGGCCCAGGCACTGTCTTTAAACACA
GAGGAAGACATTCATCGGGCTTTCCAGTCGCTTCTCACTGAAGTGAACAAGGCTGGCACACAGTACCTGCTGAGAAC
GGCCAACAGGCTCTTTGGAGAGAAAACTTGTCAGTTCCTCTCAACGTTTAAGGAATCCTGTCTTCAATTCTACCATG
CTGAGCTGAAGGAGCTTTCCTTTATCAGAGCTGCAGAAGAGTCCAGGAAACACATCAACACCTGGGTCTCAAAAAAG
ACCGAAGGTAAAATTGAAGAGTTGTTGCCGGGTAGCTCAATTGATGCAGAAACCAGGCTGGTTCTTGTCAATGCCAT
CTACTTCAAAGGAAAGTGGAATGAACCGTTTGACGAAACATACACAAGGGAAATGCCCTTTAAAATAAACCAGGAGG
AGCAAAGGCCAGTGCAGATGATGTATCAGGAGGCCACGTTTAAGCTCGCCCACGTGGGCGAGGTGCGCGCGCAGCTG
CTGGAGCTGCCCTACGCCAGGAAGGAGCTGAGCCTGCTGGTGCTGCTGCCTGACGACGGCGTGGAGCTCAGCACGGT
GGAAAAAAGTCTCACTTTTGAGAAACTCACAGCCTGGACCAAGCCAGACTGTATGAAGAGTACTGAGGTTGAAGTTC
TCCTTCCAAAATTTAAACTACAAGAGGATTATGACATGGAATCTGTGCTTCGGCATTTGGGAATTGTTGATGCCTTC
CAACAGGGCAAGGCTGACTTGTCGGCAATGTCAGCGGAGAGAGACCTGTGTCTGTCCAAGTTCGTGCACAAGAGTTT
TGTGGAGGTGAATGAAGAAGGCACCGAGGCAGCGGCAGCGTCGAGCTGCTTTGTAGTTGCAGAGTGCTGCATGGAAT
CTGGCCCCAGGTTCTGTGCTGACCACCCTTTCCTTTTCTTCATCAGGCACAACAGAGCCAACAGCATTCTGTTCTGT
GGCAGGTTCTCATCGCCATAAAGGGTGCACTTACCGTGCACTCGGCCATTTCCCTCTTCCTGTGTCCCCAGATCCCC
ACTACAGCTCCAAGAGGATGGGCCTAGAAAGCCAAGTGCAAAGATGAGGGCAGATTCTTTACCTGTCTGCCCTCATG
ATTTGCCAGCATGAATTCATGATGCTCCACACTCGCTTATGCTACTTAATCAGAATCTTGAGAAAATAGACCATAAT
GATTCCCTGTTGTATTAAAATTGCAGTCCAAATCCCATAGGATGGCAAGCAAAGTTCTTCTAGAATTCCACATGCAA
TTCACTCTGGCGACCCTGTGCTTTCCTGACACTGCGAATACATTCCTTAACCCGCTGCCTCAGTGGTAATAAATGGT
GCTAGATATTGCTACTATTTTATAGATTTCCTGGTGCTTAGCCTTATAAAAAAGGTTGTAAAATGTACATTTATATT
TTATCTTTTTTTTTTTTTTTTTTCTGAGACGCAGTCTGGCTCTCTGTCGCCCAGGCTGGAGTGCAGTGGCTCGATCT
CGGCTCACTGCAAGCTCCGCCTCCCGGGTTCACGCCATTCTCCTGCCTCAGCCTCCCGAGTAGCTGGGACTACAGGC
GCCCGCCACCACGCCCGGCTAATTTTTTGTATTTTTAGTAGAGACGGGGTTTCACCGTGTTAGCCAGGATGGTGTCG
ATCTCCTGACCTCGTGATCCACCCGCCTCGGCCTCCCAAAGTGCTGGGATTACAGGCTTGAGCCACCGCGCCCGGCT
ATATTTTATCTTTTATCTTTTTCTTTGACATTTACCAATCACCAAGCATGCACCAAACACTGCTTTAGGCACTGGGG
ACACAAAGGGGACAGAGCCATCCTCCTTTGACACCTGGTCTTCAGTTCTGTGCCCAACGTATATAGTTTTGACAATG
ACCAGGTTGGACTGTTTAATGTCTTTCAACTTACCACGTAATCCTCTTGTAGGGATCACATCTTTCTTTATGATATT
GTATTTCTCTACCTCTAACAGTAAAAATTCCATTCAACCCTTAAAGCTCACTTCAAATTCTTCTTTGAGAAGTTTTT
CCTTTCTCCGCAACCAGATGTACATATTTGAACTCTCTTTGTACTTGGAGGGCACTTCTTTCGTGGTAGTTCTTTTA
TTTTTATTAATCTCTGTATCCTTAGATAGTCCTCCAACAACCAAAGGTTGGGACTCTGTCTTACATATCTGGGTGCC
CCTCATAGTGCAGTAATAAGTAAGTTGATTATATACGAGCTATGTAACTTATATTTTTTAATGGTTGGATATCACTG
AGTTTTTTTTTTTAAGAATTTTTTTATTGAGGTAAACTTCACATAACATAAAATTAACTATTTTAAAGTGAGAAGTT
CAGTGCCACTTAGTATTGTTAACAATGTTGCATAACCACCACCTTTATTTAAAGTTCCAAAAAAAATGTTCTCCTCT
AAAAGGAAACCCCATCCCATTAAGCAGATACTCTCCATTCCTTCCTTCCTCCAGCCCCCAGCAACCACCAATCTGCT
TTCTGTCTCTATGGATTTATCTATTCTTGCTATTTTATATAAATTGAATTGTATGAGACCTTTTGTGTCTGGCTTCT
TTCACTTAGTACAAGTTTTTGAGATTTATTTACATAGTAGCATGTATCAACACTTCATTTTTATGGCCAAATAAAAT
TGTATTATGTGTTTATAGCACAATTTATTTATCCACTCATTCATTGATGGACTTTGGGTTGTTTCTGACTTTTGGCT
ATTGGGAATAGTGCTGCTATGAATGTTTGTGTACCTGTATTTGTTTGAATGCCTATTTTGCATTCTCTTGGGTATAT
ATCTAGGAGTGGAACTGCTGGGTCATATGTTAATTCTATGTTTAGCTTTTTGAGGAACAGACAAACTGTTTTCCACA
GCAGTTGAACCATTCCACATTCCCACCAGCAATGTATGAGAATTCCAATTTCTGTCCACTTCCTCACCAACACTTAT
TATTTTCCTTTTCCTTTTTTTAAAAAAAATAAGTTATGGCCATCTTAGTGGGTGTGAAGTGGTATCTCATTGTGTTT
TTTATTTGCATTTCCTATGTAATGAGCTAGAAACTAAAGTACAAACTAGATGGGACATCCAGTCCCTTTGATAGATA
ATGCTGAGTAAAAAATGAGATGAAAGACATTTGTTTGTTTTTAGAACACGAGTGACAGTTTGTTAAAAAGCTTTAGA
GGAGGAATGAAAACAAAGTGAAGTACACTTAGAAAAGGGCCAAGTGGACATCTTGGATGTCAAGTGCCTAGTTCAGT
ATCTTTTTTTTTTTTTTTTTTTTTTTTGAGACAGTGCCTCACTCTGTCACCCAGGCTGGAGTGTAGTGGCATGATCT
GGGCTCACTGCAACCTCCTCCTCCTGGATTCAAGCAATTCTCTTGCTTCAGCCTCCCAAGTAGCTGAGACTACAAGC
ACCCACCATCACACCCAGCTAATTTTGTATTTTTCAGTAGAGACGGGGTTTCGCCACATTGGCCGTGTTGGTCTTGA
ACTCCTGGCCTCAAGCGATCCGCCTACCTCAGCCTCCCAAAGTGCTAGGATTACAGGCATAAGCCACTGAGCCCAGC
CCTAGTTCAGTATCTTTTATGTAAATTACAAACATCTGCAACATTATGTATCATATGCAGATACTTATTGCATTTCT
TTTATTAGTGGTGAAAGTGTTCTATGCATTTATTGGCTCTTGAATTTCCTCATCTATGAATTGTCATTCATACACCT
ACTTTTCTGCTTCGTTTTTACATATGTCTTTGCCTATTAAAGATATTATCCCTCTGTTTTATATTTTCTCTCATTCT
TGTATTGCCTTTTAAATTTTGTTATGATGTTTCATTAATAAACAGTGTTTTGTTTTCCTCTATAATCAAAAAAAAAA
AAAAAAAAA

| | |
|---|---|
| SEQ ID NO: 47 | Human CD47 cDNA Sequence |

GGGGAGCAGGCGGGGGAGCGGGCGGGAAGCAGTGGGAGCGCGCGTGCGCGCGGCCGTGCAGCCTGGGCAGTGGGTCC
TGCCTGTGACGCGCGGCGGCGGTCGGTCCTGCCTGTAACGGCGGCGGCGGCTGCTGCTCCAGACACCTGCGGCGGCG
GCGGCGACCCCGCGGCGGGCGCGGAGATGTGGCCCCTGGTAGCGGCGCTGTTGCTGGGCTCGGCGTGCTGCGGATCA
GCTCAGCTACTATTTAATAAAACAAAATCTGTAGAATTCACGTTTTGTAATGACACTGTCGTCATTCCATGCTTTGT
TACTAATATGGAGGCACAAAACACTACTGAAGTATACGTAAAGTGGAAATTTAAAGGAAGAGATATTTACACCTTTG
ATGGAGCTCTAAACAAGTCCACTGTCCCCACTGACTTTAGTAGTGCAAAAATTGAAGTCTCACAATTACTAAAAGGA
GATGCCTCTTTGAAGATGGATAAGAGTGATGCTGTCTCACACACAGGAAACTACACTTGTGAAGTAACAGAATTAAC
CAGAGAAGGTGAAACGATCATCGAGCTAAAATATCGTGTTGTTTCATGGTTTTCTCCAAATGAAAATATTCTTATTG
TTATTTTCCCAATTTTTGCTATACTCCTGTTCTGGGGACAGTTTGGTATTAAAACACTTAAATATAGATCCGGTGGT
ATGGATGAGAAAACAATTGCTTTACTTGTTGCTGGACTAGTGATCACTGTCATTGTCATTGTTGGAGCCATTCTTTT
CGTCCCAGGTGAATATTCATTAAAGAATGCTACTGGCCTTGGTTTAATTGTGACTTCTACAGGGATATTAATATTAC
TTCACTACTATGTGTTTAGTACAGCGATTGGATTAACCTCCTTCGTCATTGCCATATTGGTTATTCAGGTGATAGCC
TATATCCTCGCTGTGGTTGGACTGAGTCTCTGTATTGCGGCGTGTATACCAATGCATGGCCCTCTTCTGATTTCAGG
TTTGAGTATCTTAGCTCTAGCACAATTACTTGGACTAGTTTATATGAAATTTGTGGCTTCCAATCAGAAGACTATAC
AACCTCCTAGGAAAGCTGTAGAGGAACCCCTTAATGCATTCAAAGAATCAAAAGGAATGATGAATGATGAATAACTG
AAGTGAAGTGATGGACTCCGATTTGGAGAGTAGTAAGACGTGAAAGGAATACACTTGTGTTTAAGCACCATGGCCTT
GATGATTCACTGTTGGGGAGAAGAAACAAGAAAAGTAACTGGTTGTCACCTATGAGACCCTTACGTGATTGTTAGTT
AAGTTTTTATTCAAAGCAGCTGTAATTTAGTTAATAAAATAATTATGATCTATGTTGTTTGCCCAATTGAGATCCAG
TTTTTTGTTGTTATTTTTAATCAATTAGGGGCAATAGTAGAATGGACAATTTCCAAGAATGATGCCTTTCAGGTCCT
AGGGCCTCTGGCCTCTAGGTAACCAGTTTAAATTGGTTCAGGGTGATAACTACTTAGCACTGCCCTGGTGATTACCC
AGAGATATCTATGAAAACCAGTGGCTTCCATCAAACCTTTGCCAACTCAGGTTCACAGCAGCTTTGGGCAGTTATGG
CAGTATGGCATTAGCTGAGAGGTGTCTGCCACTTCTGGGTCAATGGAATAATAAATTAAGTACAGGCAGGAATTTGG
TTGGGAGCATCTTGTATGATCTCCGTATGATGTGATATTGATGGAGATAGTGGTCCTCATTCTTGGGGGTTGCCATT
CCCACATTCCCCCTTCAACAAACAGTGTAACAGGTCCTTCCCAGATTTAGGGTACTTTTATTGATGGATATGTTTTC
CTTTTATTCACATAACCCCTTGAAACCCTGTCTTGTCCTCCTGTTACTTGCTTCTGCTGTACAAGATGTAGCACCTT
TTCTCCTCTTTGAACATGGTCTAGTGACACGGTAGCACCAGTTGCAGGAAGGAGCCAGACTTGTTCTCAGAGCACTG
TGTTCACACTTTTCAGCAAAAATAGCTATGGTTGTAACATATGTATTCCCTTCCTCTGATTTGAAGGCAAAAATCTA
CAGTGTTTCTTCACTTCTTTTCTGATCTGGGGCATGAAAAAAGCAAGATTGAAATTTGAACTATGAGTCTCCTGCAT
GGCAACAAAATGTGTGTCACCATCAGGCCAACAGGCCAGCCCTTGAATGGGGATTTATTACTGTTGTATCTATGTTG
CATGATAAACATTCATCACCTTCCTCCTGTAGTCCTGCCTCGTACTCCCCTTCCCCTATGATTGAAAAGTAAACAAA
ACCCACATTTCCTATCCTGGTTAGAAGAAAATTAATGTTCTGACAGTTGTGATCGCCTGGAGTACTTTTAGACTTTT
AGCATTCGTTTTTTACCTGTTTGTGGATGTGTGTTTGTATGTGCATACGTATGAGATAGGCACATGCATCTTCTGTA
TGGACAAAGGTGGGGTACCTACAGGAGAGCAAAGGTTAATTTTGTGCTTTTAGTAAAAACATTTAAATACAAAGTTC
TTTATTGGGTGGAATTATATTTGATGCAAATATTTGATCACTTAAAACTTTTAAAACTTCTAGGTAATTTGCCACGC
TTTTTGACTGCTCACCAATACCCTGTAAAAATACGTAATTCTTCCTGTTTGTGTAATAAGATATTCATATTTGTAGT
TGCATTAATAATAGTTATTTCTTAGTCCATCAGATGTTCCCGTGTGCCTCTTTTATGCCAAATTGATTGTCATATTT
CATGTTGGGACCAAGTAGTTTGCCCATGGCAAACCTAAATTTATGACCTGCTGAGGCCTCTCAGAAAACTGAGCATA
CTAGCAAGACAGCTCTTCTTGAAAAAAAAAATATGTATACACAAATATATACGTATATCTATATATACGTATGTATA
TACACACATGTATATTCTTCCTTGATTGTGTAGCTGTCCAAAATAATAACATATATAGAGGGAGCTGTATTCCTTTA
TACAAATCTGATGGCTCCTGCAGCACTTTTTCCTTCTGAAAATATTTACATTTTGCTAACCTAGTTTGTTACTTTAA
AAATCAGTTTTGATGAAAGGAGGGAAAAGCAGATGGACTTGAAAAAGATCCAAGCTCCTATTAGAAAAGGTATGAAA
ATCTTTATAGTAAAATTTTTTATAAACTAAAGTTGTACCTTTTAATATGTAGTAAACTCTCATTTATTTGGGGTTCG
CTCTTGGATCTCATCCATCCATTGTGTTCTCTTTAATGCTGCCTGCCTTTTGAGGCATTCACTGCCCTAGACAATGC
CACCAGAGATAGTGGGGGAAATGCCAGATGAAACCAACTCTTGCTCTCACTAGTTGTCAGCTTCTCTGGATAAGTGA
CCACAGAAGCAGGAGTCCTCCTGCTTGGGCATCATTGGGCCAGTTCCTTCTCTTTAAATCAGATTTGTAATGGCTCC
CAAATTCCATCACATCACATTTAAATTGCAGACAGTGTTTTGCACATCATGTATCTGTTTTGTCCCATAATATGCTT
TTTACTCCCTGATCCCAGTTTCTGCTGTTGACTCTTCCATTCAGTTTTATTTATTGTGTGTTCTCACAGTGACACCA
TTTGTCCTTTTCTGCAACAACCTTTCCAGCTACTTTTGCCAAATTCTATTTGTCTTCTCCTTCAAAACATTCTCCTT
TGCAGTTCCTCTTCATCTGTGTAGCTGCTCTTTTGTCTCTTAACTTACCATTCCTATAGTACTTTATGCATCTCTGC
TTAGTTCTATTAGTTTTTTGGCCTTGCTCTTCTCCTTGATTTTAAAATTCCTTCTATAGCTAGAGCTTTTCTTTCTT
TCATTCTCTCTTCCTGCAGTGTTTTGCATACATCAGAAGCTAGGTACATAAGTTAAATGATTGAGAGTTGGCTGTAT
TTAGATTTATCACTTTTTAATAGGGTGAGCTTGAGAGTTTTCTTTCTTTCTGTTTTTTTTTTTTGTTTTTTTTTTTT
TTTTTTTTTTTTTTTTTTTTGACTAATTTCACATGCTCTAAAAACCTTCAAAGGTGATTATTTTTCTCCTGGAAACT
CCAGGTCCATTCTGTTTAAATCCCTAAGAATGTCAGAATTAAAATAACAGGGCTATCCCGTAATTGGAAATATTTCT
TTTTTCAGGATGCTATAGTCAATTTAGTAAGTGACCACCAAATTGTTATTTGCACTAACAAAGCTCAAAACACGATA
AGTTTACTCCTCCATCTCAGTAATAAAAATTAAGCTGTAATCAACCTTCTAGGTTTCTCTTGTCTTAAAATGGGTAT
TCAAAAATGGGGATCTGTGGTGTATGTATGGAAACACATACTCCTTAATTTACCTGTTGTTGGAAACTGGAGAAATG
ATTGTCGGGCAACCGTTTATTTTTTATTGTATTTTATTTGGTTGAGGGATTTTTTTATAAACAGTTTTACTTGTGTC
ATATTTTAAAATTACTAACTGCCATCACCTGCTGGGGTCCTTTGTTAGGTCATTTTCAGTGACTAATAGGGATAATC
CAGGTAACTTTGAAGAGATGAGCAGTGAGTGACCAGGCAGTTTTTCTGCCTTTAGCTTTGACAGTTCTTAATTAAGA
TCATTGAAGACCAGCTTTCTCATAAATTTCTCTTTTTGAAAAAAAGAAAGCATTTGTACTAAGCTCCTCTGTAAGAC
AACATCTTAAATCTTAAAAGTGTTGTTATCATGACTGGTGAGAGAAGAAAACATTTTGTTTTTATTAAATGGAGCAT
TATTTACAAAAAGCCATTGTTGAGAATTAGATCCCACATCGTATAAATATCTATTAACCATTCTAAATAAAGAGAAC
TCCAGTGTTGCTATGTGCAAGATCCTCTCTTGGAGCTTTTTTGCATAGCAATTAAAGGTGTGCTATTTGTCAGTAGC
CATTTTTTTGCAGTGATTTGAAGACCAAAGTTGTTTTACAGCTGTGTTACCGTTAAAGGTTTTTTTTTTTATATGTA
TTAAATCAATTTATCACTGTTTAAAGCTTTGAATATCTGCAATCTTTGCCAAGGTACTTTTTTATTTAAAAAAAAAC
ATAACTTTGTAAATATTACCCTGTAATATTATATATACTTAATAAAACATTTTAAGCTATTTTGTTGGGCTATTTCT
ATTGCTGCTACAGCAGACCACAAGCACATTTCTGAAAAATTTAATTTATTAATGTATTTTTAAGTTGCTTATATTCT
AGGTAACAATGTAAAGAATGATTTAAAATATTAATTATGAATTTTTTGAGTATAATACCCAATAAGCTTTTAATTAG
AGCAGAGTTTTAATTAAAAGTTTTAAATCAGTC

| | |
|---|---|
| SEQ ID NO: 48 | Human galectin-9 cDNA Sequence |

TCCCCATTGAATAACAGCCAAGTTGCTTTGGTTTCTATTTCTTTGTTAAGTCGTTCCCTCTACAAAGGACTTCCTAG
TGGGTGTGAAAGGCAGCGGTGGCCACAGAGGCGGCGGAGAGATGGCCTTCAGCGGTTCCCAGGCTCCCTACCTGAGT
CCAGCTGTCCCCTTTTCTGGGACTATTCAAGGAGGTCTCCAGGACGGACTTCAGATCACTGTCAATGGGACCGTTCT
CAGCTCCAGTGGAACCAGGTTTGCTGTGAACTTTCAGACTGGCTTCAGTGGAAATGACATTGCCTTCCACTTCAACC
CTCGGTTTGAAGATGGAGGGTACGTGGTGTGCAACACGAGGCAGAACGGAAGCTGGGGGCCCGAGGAGAGGAAGACA
CACATGCCTTTCCAGAAGGGGATGCCCTTTGACCTCTGCTTCCTGGTGCAGAGCTCAGATTTCAAGGTGATGGTGAA
CGGGATCCTCTTCGTGCAGTACTTCCACCGCGTGCCCTTCCACCGTGTGGACACCATCTCCGTCAATGGCTCTGTGC
AGCTGTCCTACATCAGCTTCCAGAACCCCCGCACAGTCCCTGTTCAGCCTGCCTTCTCCACGGTGCCGTTCTCCCAG
CCTGTCTGTTTCCCACCCAGGCCCAGGGGGCGCAGACAAAAACCTCCCGGCGTGTGGCCTGCCAACCCGGCTCCCAT
TACCCAGACAGTCATCCACACAGTGCAGAGCGCCCCTGGACAGATGTTCTCTACTCCCGCCATCCCACCTATGATGT
ACCCCCACCCCGCCTATCCGATGCCTTTCATCACCACCATTCTGGGAGGGCTGTACCCATCCAAGTCCATCCTCCTG
TCAGGCACTGTCCTGCCCAGTGCTCAGAGGTTCCACATCAACCTGTGCTCTGGGAACCACATCGCCTTCCACCTGAA
CCCCCGTTTTGATGAGAATGCTGTGGTCCGCAACACCCAGATCGACAACTCCTGGGGGTCTGAGGAGCGAAGTCTGC
CCCGAAAAATGCCCTTCGTCCGTGGCCAGAGCTTCTCAGTGTGGATCTTGTGTGAAGCTCACTGCCTCAAGGTGGCC
GTGGATGGTCAGCACCTGTTTGAATACTACCATCGCCTGAGGAACCTGCCCACCATCAACAGACTGGAAGTGGGGGG
CGACATCCAGCTGACCCATGTGCAGACATAGGCGGCTTCCTGGCCCTGGGGCCGGGGGCTGGGGTGTGGGGCAGTCT
GGGTCCTCTCATCATCCCCACTTCCCAGGCCCAGCCTTTCCAACCCTGCCTGGGATCTGGGCTTTAATGCAGAGGCC
ATGTCCTTGTCTGGTCCTGCTTCTGGCTACAGCCACCCTGGAACGGAGAAGGCAGCTGACGGGGATTGCCTTCCTCA
GCCGCAGCAGCACCTGGGGCTCCAGCTGCTGGAATCCTACCATCCCAGGAGGCAGGCACAGCCAGGGAGAGGGGAGG
AGTGGGCAGTGAAGATGAAGCCCCATGCTCAGTCCCCTCCCATCCCCCACGCAGCTCCACCCCAGTCCCAAGCCACC
AGCTGTCTGCTCCTGGTGGGAGGTGGCCTCCTCAGCCCCTCCTCTCTGACCTTTAACCTCACTCTCACCTTGCACCG
TGCACCAACCCTTCACCCCTCCTGGAAAGCAGGCCTGATGGCTTCCCACTGGCCTCCACCACCTGACCAGAGTGTTC
TCTTCAGAGGACTGGCTCCTTTCCCAGTGTCCTTAAAATAAAGAAATGAAAATGCTTGTTGGCACATTCAAAAAAAA
AAAAAAAAAAAAAAAAAAAAAAAAAAAAAAAAAAAAAAAAAAAAAAAAAAAAAAAAAAAAAAAA

| | |
|---|---|
| SEQ ID NO: 49 | CD46 Protein Sequence |

MMAFCALRKALPCRPENPFSSRCFVEILWVSLALVFLLPMPSDACDEPPKFESMRPQFLNTTYRPGDRVEYECRPGF
QPMVPALPTFSVCQDDNTWSPLQEACRRKACSNLPDPLNGQVSYPNGDMLFGSKAQFTCNTGFYIIGAETVYCQVSG
NVMAWSEPSPLCEKILCKPPGEIPNGKYTNSHKDVFEYNEVVTYSCLSSTGPDEFSLVGESSLFCIGKDEWSSDPPE
CKVVKCPYPVVPNGEIVSGFGSKFYYKAEVVFKCNAGFTLHGRDTIVCGANSTWEPEMPQCIKDSKPTDPPATPGPS
HPGPPSPSDASPPKDAESLDGGIIAAIVVGVLAAIAVIAGGVYFFHHKYNKKRSK

| | |
|---|---|
| SEQ ID NO: 50 | CD55 Protein Sequence |

MSPLPRSAPAVRRLMGGQTPPPLLLLLLLLCIPAAQGDCSLPPDVPNAQPDLRGLASFPEQTTITYKCNKGFVKVPG
MADSVLCLNDKWSEVAEFCNRSCDVPTRLHFASLKKSYSKQNYFPEGFTVEYECRKGYKRDLTLSEKLTCLQNFTWS
KPDEFCKKKQCPTPGELKNGHVNITTDLLFGASIFFSCNAGYRLVGATSSYCFAIANDVEWSDPLPECQEISPTVKA
IPAVEKPITVNFPATKYPAIPRATTSFHSSTSKNRGNPSSGMRIMSSGTMLLIAGGVAVIIIIVALILAKGFWHYGK
SGSYHTHENNKAVNVAFYNLPATGDAADVRPGN

| | |
|---|---|
| SEQ ID NO: 51 | CD59 Protein Sequence |

MGSKGGFILLWLLSILAVLCHLGHSLQCYNCINPAGSCTTAMNCSHNQDACIFVEAVPPKTYYQCWRFDECNFDFIS
RNLAEKKLKYNCCRKDLCNKSDATISSGKTALLVILLLVATWHFCL

| | |
|---|---|
| SEQ ID NO: 52 | ICP47 Protein Sequence |

MSWALKTTDMFLDSSRCTHRTYGDVCAEIHKREREDREAARTAVTDPELPLLCPPDVRSDPASRNPTQQTRGCARSN
ERQDRVLAP

| | |
|---|---|
| SEQ ID NO: 53 | HLA-G1 Protein Sequence |

MVVMAPRTLFLLLSGALTLTETWAGSHSMRYFSAAVSRPGRGEPRFIAMGYVDDTQFVRFDSDSACPRMEPRAPWVE
QEGPEYWEEETRNTKAHAQTDRMNLQTLRGYYNQSEASSHTLQWMIGCDLGSDGRLLRGYEQYAYDGKDYLALNEDL
RSWTAADTAAQISKRKCEAANVAEQRRAYLEGTCVEWLHRYLENGKEMLQRADPPKTHVTHHPVFDYEATLRCWALG
FYPAEIILTWQRDGEDQTQDVELVETRPAGDGTFQKWAAVVVPSGEEQRYTCHVQHEGLPEPLMLRWKQSSLPTIPI
MGIVAGLVVLAAVVTGAAVAAVLWRKKSSD

| | |
|---|---|
| SEQ ID NO: 54 | HLA-E Protein Sequence |

MVDGTLLLLLSEALALTQTWAGSHSLKYFHTSVSRPGRGEPRFISVGYVDDTQFVRFDNDAASPRMVPRAPWMEQEG
SEYWDRETRSARDTAQIFRVNLRTLRGYYNQSEAGSHTLQWMHGCELGPDGRFLRGYEQFAYDGKDYLTLNEDLRSW
TAVDTAAQISEQKSNDASEAEHQRAYLEDTCVEWLHKYLEKGKETLLHLEPPKTHVTHHPISDHEATLRCWALGFYP
AEITLTWQQDGEGHTQDTELVETRPAGDGTFQKWAAVVVPSGEEQRYTCHVQHEGLPEPVTLRWKPASQPTIPIVGI
IAGLVLLGSVVSGAVVAAVIWRKKSSGGKGGSYSKAEWSDSAQGSESHSL

| | |
|---|---|
| SEQ ID NO: 55 | Human β-2-microglobulin Protein Sequence |

MSRSVALAVLALLSLSGLEAIQRTPKIQVYSRHPAENGKSNFLNCYVSGFHPSDIEVDLLKNGERIEKVEHSDLSFS
KDWSFYLLYYTEFTPTEKDEYACRVNHVTLSQPKIVKWDRDM

| | |
|---|---|
| SEQ ID NO: 56 | Human PD-L1 Protein Sequence |

MRIFAVFIFMTYWHLLNAPYNKINQRILVVDPVTSEHELTCQAEGYPKAEVIWTSSDHQVLSGKTTTTNSKREEKLF
NVTSTLRINTTTNEIFYCTFRRLDPEENHTAELVIPELPLAHPPNERTHLVILGAILLCLGVALTFIFRLRKGRMMD
VKKCGIQDTNSKKQSDTHLEET

| | |
|---|---|
| SEQ ID NO: 57 | Human PD-L2 Protein Sequence |

MIFLLLMLSLELQLHQIAALFTVTVPKELYIIEHGSNVTLECNFDTGSHVNLGAITASLQKVENDTSPHRERATLLE
EQLPLGKASFHIPQVQVRDEGQYQCIIIYGVAWDYKYLTLKVKASYRKINTHILKVPETDEVELTCQATGYPLAEVS
WPNVSVPANTSHSRTPEGLYQVTSVLRLKPPPGRNFSCVFWNTHVRELTLASIDLQSQMEPRTHPTWLLHIFIPFCI
IAFIFIATVIALRKQLCQKLYSSKDTTKRPVTTTKREVNSAI

| | |
|---|---|
| SEQ ID NO: 58 | Human Spi9 Protein Sequence |

METLSNASGTFAIRLLKILCQDNPSHNVFCSPVSISSALAMVLLGAKGNTATQMAQALSLNTEEDIHRAFQSLLTEV
NKAGTQYLLRTANRLFGEKTCQFLSTFKESCLQFYHAELKELSFIRAAEESRKHINTWVSKKTEGKIEELLPGSSID
AETRLVLVNAIYFKGKWNEPFDETYTREMPFKINQEEQRPVQMMYQEATFKLAHVGEVRAQLLELPYARKELSLLVL
LPDDGVELSTVEKSLTFEKLTAWTKPDCMKSTEVEVLLPKFKLQEDYDMESVLRHLGIVDAFQQGKADLSAMSAERD
LCLSKFVHKSFVEVNEEGTEAAAASSCFVVAECCMESGPRFCADHPFLFFIRHNRANSILFCGRFSSP

| | |
|---|---|
| SEQ ID NO: 59 | Human CD47 Protein Sequence |

MWPLVAALLLGSACCGSAQLLFNKTKSVEFTFCNDTVVIPCFVTNMEAQNTTEVYVKWKFKGRDIYTFDGALNKSTV
PTDFSSAKIEVSQLLKGDASLKMDKSDAVSHTGNYTCEVTELTREGETIIELKYRVVSWFSPNENILIVIFPIFAIL
LFWGQFGIKTLKYRSGGMDEKTIALLVAGLVITVIVIVGAILFVPGEYSLKNATGLGLIVTSTGILILLHYYVFSTA
IGLTSFVIAILVIQVIAYILAVVGLSLCIAACIPMHGPLLISGLSILALAQLLGLVYMKFVASNQKTIQPPRKAVEE
PLNAFKESKGMMNDE

| | |
|---|---|
| SEQ ID NO: 60 | Human galectin-9 Protein Sequence |

MAFSGSQAPYLSPAVPFSGTIQGGLQDGLQITVNGTVLSSSGTRFAVNFQTGFSGNDIAFHFNPRFEDGGYVVCNTR
QNGSWGPEERKTHMPFQKGMPFDLCFLVQSSDFKVMVNGILFVQYFHRVPFHRVDTISVNGSVQLSYISFQNPRTVP
VQPAFSTVPFSQPVCFPPRPRGRRQKPPGVWPANPAPITQTVIHTVQSAPGQMFSTPAIPPMMYPHPAYPMPFITTI
LGGLYPSKSILLSGTVLPSAQRFHINLCSGNHIAFHLNPRFDENAVVRNTQIDNSWGSEERSLPRKMPFVRGQSFSV
WILCEAHCLKVAVDGQHLFEYYHRLRNLPTINRLEVGGDIQLTHVQT

While some embodiments have been shown and described herein, such embodiments are provided by way of example only. Numerous variations, changes, and substitutions will now occur to those skilled in the art without departing from the invention. It should be understood that various alternatives to the embodiments of the invention described herein will be employed in practicing the invention.

1.-147. (canceled)

148. A genetically modified non-human animal comprising an exogenous nucleic acid sequence at least 95% identical to SEQ ID NO: 359 or SEQ ID NO: 502.

149. The genetically modified non-human animal of claim 148, wherein the exogenous nucleic acid sequence is at least 96% identical to SEQ ID NO: 359 or SEQ ID NO: 502.

150. The genetically modified non-human animal of claim 148, wherein the exogenous nucleic acid sequence is at least 99% identical to SEQ ID NO: 359 or SEQ ID NO: 502.

151. The genetically modified non-human animal of claim 148, wherein the exogenous nucleic acid is 100% identical to SEQ ID NO: 0.359.

152. The genetically modified non-human animal of claim 148, wherein the exogenous nucleic acid is 100% identical to SEQ ID NO: 502.

153. The genetically modified non-human animal of claim 148, further comprising an exogenous nucleic acid sequence that encodes for a β-2-microglobulin (B2M) protein.

154. The genetically modified non-human animal of claim 148, further comprising an exogenous nucleic acid sequence that encodes for a β-2-microglobulin (B2M) protein, wherein the B2M protein is fused to a protein encoded by the exogenous nucleic acid sequence that is at least 95% identical to SEQ ID NO: 359 or SEQ ID NO: 502.

155. (canceled)

156. The genetically modified non-human animal of claim 148, wherein the exogenous nucleic acid sequence is inserted in the genetically modified non-human animal's genome at a site effective to reduce expression of a glycoprotein galactosyltransferase alpha 1,3 (GGTA1), a putative cytidine monophosphate-N-acetylneuraminic acid hydroxylase-like protein (CMAH), a β1,4 N-acetylgalactosaminyltransferase (B4GALNT2), a C—X—C motif chemokine 10 (CXCL10), a MEC class I polypeptide-related sequence A (MICA), a MEC class I polypeptide-related sequence B (WEB), a transporter associated with antigen processing 1 (TAP1), a NOD-like receptor family CARD domain containing 5 (NLRC5), or a combination thereof, in comparison to: a non-human animal of the same species without the exogenous nucleic acid sequence, or a non-human animal of the same species with the exogenous nucleic acid inserted in a different site.

157. A genetically modified non-human cell, tissue, or organ comprising an exogenous nucleic acid sequence at least 95% identical to SEQ ID NO: 359 or SEQ ID NO: 502.

158. The genetically modified non-human cell, tissue, or organ of claim 157, wherein the exogenous nucleic acid sequence is at least 96% identical to SEQ ID NO: 359 or SEQ ID NO: 502.

159. The genetically modified non-human cell, tissue, or organ of claim 157, wherein the exogenous nucleic acid sequence is at least 99% identical to SEQ ID NO: 359 or SEQ ID NO: 502.

160. The genetically modified non-human cell, tissue, or organ of claim 157, wherein the exogenous nucleic acid sequence is 100% identical to SEQ ID NO: 359.

161. The genetically modified non-human cell, tissue, or organ of claim 157, wherein the exogenous nucleic acid sequence is 100% identical to SEQ ID NO: 502.

162. The genetically modified non-human cell, tissue, or organ of claim 157, further comprising an exogenous nucleic acid sequence that encodes for a β-2-microglobulin (B2M) protein.

163. The genetically modified non-human cell, tissue, or organ of claim 157, further comprising an exogenous nucleic acid sequence that encodes for a β-2-microglobulin (B2M) protein-, wherein the B2M protein is fused to a protein encoded by the exogenous nucleic acid sequence that is at least 95% identical to SEQ ID NO: 359 or SEQ ID NO: 502.

164. The genetically modified non-human cell, tissue, or organ of claim 157, wherein the exogenous nucleic acid sequence is inserted in the genetically modified non-human cell, tissue, or organ's genome at a site effective to reduce expression of a glycoprotein galactosyltransferase alpha 1,3 (GGTA1), a putative cytidine monophosphate-N-acetylneuraminic acid hydroxylase-like protein (CMAH), a β1,4 N-acetylgalactosaminyltransferase (B4GALNT2), a C—X—C motif chemokine 10 (CXCL10), a MHC class I polypeptide-related sequence A (MICA), a MHC class I polypeptide-related sequence B (MICB), a transporter associated with antigen processing 1 (TAP1), a NOD-like receptor family CARD domain containing 5 (NLRC5), or a combination thereof, in comparison to: a genetically modified non-human cell, tissue, or organ of the same species without the exogenous nucleic acid sequence or a genetically modified non-human cell, tissue, or organ with the exogenous nucleic acid inserted in a different site.

165. The genetically modified non-human cell, tissue, or organ of claim 157, wherein the genetically modified non-human cell is an islet cell.

166. The genetically modified non-human cell, tissue, or organ of claim 157, wherein the genetically modified non-human cell is a stem cell.

167. The genetically modified non-human cell, tissue, or organ of claim 157, wherein the genetically modified non-human tissue is a solid organ transplant.

168. The genetically modified non-human cell, tissue, or organ of claim 157, wherein the genetically modified non-human tissue is all or a portion of a liver.

169. The genetically modified non-human cell, tissue, or organ of claim 157, wherein the genetically modified non-human tissue is all or a portion of a kidney.

170. A method comprising providing to a subject, the genetically modified non-human cell, tissue or organ of claim 157.

171. The method of claim 170, further comprising providing to the subject a tolerizing vaccine.

172. The method of claim 170, further comprising providing to the subject an anti-CD40 agent.

173. A method of making a genetically modified pig comprising:
a) obtaining a porcine fetal fibroblast cell comprising—an exogenous nucleic acid sequence at least 95% identical to SEQ ID NO: 359 or SEQ ID NO: 502;
b) genetically modifying said porcine fetal fibroblast cell using clustered regularly interspaced short palindromic repeats (CRISPR)/Cas by (i) disrupting a gene encoding a glycoprotein galactosyltransferase alpha 1,3 (GGTA1) in the porcine fetal fibroblast cell comprising the exogenous nucleic acid sequence, or (ii) inserting the exogenous nucleic acid sequence at least 95% identical to SEQ ID NO: 359 or SEQ ID NO: 502 in the porcine fetal fibroblast cell comprising the disrupted gene encoding the GGTA1;
c) transferring a nucleus of the genetically modified porcine fetal fibroblast cell to a porcine enucleated oocyte to generate an embryo; and
d) transferring the embryo into a surrogate pig and growing the transferred embryo to the genetically modified pig in the surrogate pig.

174. The genetically modified non-human animal of claim 148, wherein the non-human animal is a pig.

175. The genetically modified non-human cell, tissue, or organ of claim 157, wherein the non-human cell, tissue or organ is a pig cell, tissue or organ, respectively.
